(19) United States
(12) Patent Application Publication
(10) Pub. No.: US 2023/0159913 A1
(43) Pub. Date: May 25, 2023

(54) TARGETED BASE EDITING OF THE USH2A GENE

(71) Applicants: The Broad Institute, Inc., Cambridge, MA (US); President and Fellows of Harvard College, Cambridge, MA (US); Massachusetts Eye and Ear Infirmary, Boston, MA (US)

(72) Inventors: David R. Liu, Cambridge, MA (US); Jonathan Ma Levy, Cambridge, MA (US); Wei Hsi Yeh, Cambridge, MA (US); Jason Comander, Boston, MA (US); Yuki Tachida, Boston, MA (US); Eric A. Pierce, Boston, MA (US); Qin Liu, Boston, MA (US); Nachiket Pendse, Boston, MA (US)

(73) Assignees: The Broad Institute, Inc., Cambridge, MA (US); President and Fellows of Harvard College, Cambridge, MA (US); Massachusetts Eye and Ear Infirmary, Boston, MA (US)

(21) Appl. No.: 17/921,971

(22) PCT Filed: Apr. 27, 2021
(86) PCT No.: PCT/US2021/029488

§ 371 Date: Oct. 27, 2022

Related U.S. Application Data
(60) Provisional application No. 63/016,929, filed on Apr. 28, 2020.

(51) Int. Cl. C12N 15/10; C12N 15/113; C12N 9/78

(52) U.S. Cl. CPC C12N 15/102; C12N 15/113; C12N 9/78; C12N 2310/20; C12Y 305/04004; C12Y 305/04005

(57) ABSTRACT

The disclosure provides methods of deaminating adenosine and cytosine bases in a target nucleic acid sequence in an USH2A gene comprising contacting the USH2A gene with a base editor in association with a guide RNA (gRNA). In some aspects, base editing is used to restore US2HA function by disrupting a splice site in the USH2A gene sequence to induce skipping of an exon containing a mutation, while in other embodiments, base editing is used to restore US2HA function by correcting a point mutation e.g., in an exon) so as to correct mutations. The disclosure also provides complexes of adenosine base editors and guide RNAs, and complexes of cytidine base editors and guide RNAs. The disclosure further provides pharmaceutical compositions and cells comprising these complexes. The disclosure also provides vectors encoding these complexes, base editors, and gRNAs. In some embodiments, the methods and compositions provided herein are used to treat Usher syndrome and autosomal recessive retinitis pigmentosa (arRP).

### RELATED APPLICATIONS

This application claims priority under 35 U.S.C. § 119(e) to U.S. Provisional Application, U.S. Ser. No. 63/016,929, filed Apr. 28, 2020, which is incorporated herein by reference.

### BACKGROUND OF THE INVENTION

The USH2A gene encodes the transmembrane protein Usherin. Usherin localizes mainly at the periciliary region of mammalian photoreceptors and at the stereocilia or hair bundle of the inner ear hair cells (see, Maerker et al. Hum Mol Genet 2008:17(1):71-86; Liu et al., Proc Natl Acad Sci USA. 2007; 104(11):4413-8). The Usherin protein has a large extracellular domain that is proposed to interact with basement membrane collagen IV and fibronectin via laminin domains (see Maerker et al., 2008: Remers et al. Hum Mol Genet. 2003; 14(24):3933-43). Usherin also interacts with other proteins of the USH1 and USH2 complex to form Usher networks (Hum Mol Genet, 26, 1157-1172).

Mutations in USH2A are the most common cause of both Usher syndrome type II and autosomal recessive retinitis pigmentosa (arRP), accounting for approximately 17% of the recessive RP cases. The impairment of both vision and hearing in Usher syndrome results in a reduced ability of the individual to perceive, communicate, and extract vital information from the environment. Longitudinal regression analysis has showed that the disease course for patients with USH2A mutations can be rapidly progressive, particularly with respect to losing visual field and mobility.

While USH2A-targeted therapies have been developed and shown to have efficacy in preclinical models in vitro and in vivo, such therapies by their nature are not curative and may require repeat dosing to remain effective. Alternative genetic approaches to targeting USH2A that are more curative in nature are highly desired and would significantly advance the art.

### SUMMARY OF THE INVENTION

Provided herein are methods, complexes, vectors, compositions, and kits for modifying a polynucleotide (e.g., genomic DNA) using a complex comprising a fusion protein comprising a deaminase (e.g., cytidine deaminase or adenosine deaminase) and a nucleic acid programmable DNA binding protein (“napDNAbp”), and a guide RNA, to treat Usher syndrome. Usher syndrome patients present with congenital, bilateral sensorineural hearing loss; progressive, bilateral, night blindness; or constricted visual fields (tunnel vision). Nearly 80% of Usher syndrome cases are associated with the USH2A gene, which encodes the Usherin protein. Usherin is part of Usher protein complex II that is present in photoreceptor cells of the retina and hair cells of the inner ear. Usher syndrome has autosomal recessive heredity.

In some aspects, the present disclosure provides methods of base editing the USH2A gene locus using a base editor that comprises a napDNAbp domain and one or more deaminase domains (e.g., a cytidine or adenosine deaminase), which when complexed with a suitable guide RNA, are effective in treating Usher syndrome (e.g., Usher syndrome, type 2A) by installing one or more edits (e.g., insertion, deletion, or replacement of one or more nucleobases) in the USH2A gene locus. In some embodiments, the one or more nucleobase edits installed in the USH2A gene locus result in a modified USH2A gene product having increased stability, activity, and/or production relative to a non-mutated Usherin protein. In various embodiments, the genome editing strategies disclosed herein can be implemented to target the editing of specific nucleobase positions in the USH2A gene locus, which when edited, impart the modified USH2A gene product with increased stability, activity, and/or production.

In some embodiments, deaminating an adenosine nucleobase in the USH2A gene ameliorates one or more symptoms of Usher syndrome, such as partially or completely reversing visual loss and/or hearing loss. In other embodiments, deaminating a cytosine nucleobase in the USH2A gene ameliorates one or more symptoms of Usher syndrome, such as partially or completely reversing visual loss and/or hearing loss. In some embodiments, the step of contacting, or deaminating of an adenosine nucleobase in the USH2A gene, partially or completely restores auditory function, e.g., by restoring ciliogenesis. In some embodiments, deaminating a cytosine nucleobase in the USH2A gene partially or completely restores auditory function, e.g., by restoring ciliogenesis.

Despite the success of pre-clinical and clinical studies of AAV-mediated gene augmentation therapy for multiple genetic types of inherited retinal degeneration, developing gene therapies for USH2A-associated retinitis pigmentosa has been challenging because the large size of the USH2A coding sequence (15602 base pairs (bp), coding for 5202 amino acids), far exceeds the packaging capacity of commonly used AAV viral delivery vectors. The present disclosure overcome these translational barriers by using a CRISPR/Cas base editing approach for USH2A-associated diseases, such as autosomal recessive retinitis pigmentosa. CRISPR/Cas systems are capable of maintaining the edited gene under its endogenous regulatory elements by directly altering the genomic USH2A loci, thereby avoiding ectopic expression and abnormal gene production that may occur with conventional viral-mediated gene augmentation therapies.

To e nable the direct installation or correction of point mutations in living cells, engineered proteins that directly convert a target base pair to a different base pair without creating double-stranded DNA breaks, known as base editors, have recently been developed. The base editors useful in the present disclosure include cytidine base editors and adenine base editors, which are fusion proteins containing a cytosine deaminase or adenosine deaminase, respectively. Cytosine base editors (CBEs), such as BE4max, catalyze the conversion of target CG base pairs to T⋅A, while adenine base editors (ABEs), such as ABEmax (also referred to herein as ABE 7.10), convert target A⋅T base pairs to G⋅C. Base editing with canonical base editors requires the presence of a PAM located approximately 15 base pairs from the target nucleotide(s). Each programmable DNA-binding protein domain recognizes a different PAM sequence. Only about one quarter of pathogenic transition point mutations have a suitably located canonical PAM “NGG” sequence that is compatible with S. pyogenes Cas9-derived base editors. Cytidine deaminases have proven to be broadly compatible with many Cas homologs including SaCas9, SaCas9-KKH, Cas12a (Cpf1), SpCas9-NG, and circularly permuted CP-Cas9s, greatly expanding their targeting scope. Existing adenosine deaminases have unfortunately shown limited compatibility with Cas homologs. Some Cas homologs, such as SaCas9, SaCas9-KKH, SpCas9-NG, and CP-Cas9, are compatible with ABEs, but editing efficiencies are substantially lower than those of the corresponding CBEs. Reference is made to Komor, A.C. et al., Improved base excision repair inhibition and bacteriophage Mu Gam protein yields C:G-to-T:A base editors with higher efficiency and product purity, Sci Adv 3 (2017); Rees, H.A. et al., Improving the DNA specificity and applicability of base editing through protein engineering and protein delivery, Nat. Commun. 8, 15790 (2017); U.S. Patent Application Publication No. 2018/0073012, published Mar. 15, 2018; U.S. Patent Application Publication No. 2017/0121693, published May 4, 2017; International Publication No. WO 2017/070633, published Apr. 27, 2017; and U.S. Patent Publication No. 2015/0166980, published Jun. 18, 2015, U.S. Pat. No. 9,840,699, issued Dec. 12, 2017; and U.S. Pat. No. 10,077,453, issued Sep. 18, 2018, each of which is incorporated herein by reference.

In certain embodiments, the presently disclosed systems, compositions, kits, uses, and methods make use of base editors BE4max, BE4max-VRQR, BE4-SaKKH, ABE7.10, ABE8e, ABE8e(V106W), ABE8-NG, ABE8-VQR, and ABE-xCas9 to install targeted edits in the USH2A gene. In certain embodiments, the presently disclosed systems, compositions, kits, uses, and methods make use of these base editors to install targeted edits in the USH2A gene with low frequency of off-target effects.

Accordingly, aspects of the disclosure provide methods and compositions for deaminating an adenine nucleobase (A) in an USH2A gene using a complex of an adenine base editor (ABE) bound to a guide RNA (gRNA), wherein the gRNA comprises a guide sequence that is complementary to a target nucleic acid sequence in the USH2A gene.

Other aspects of the disclosure provide methods and compositions for deaminating a cytosine nucleobase (C) in an USH2A gene using a complex of a cytidine base editor (CBE) bound to a guide RNA (gRNA), wherein the gRNA comprises a guide sequence that is complementary to a target nucleic acid sequence in the USH2A gene.

In some embodiments, the disclosure provides complexes comprising a base editor (e.g., adenine base editor) and a guide RNA (gRNA). In some embodiments, the gRNA directs the base editor in proximity to a base in the USH2A gene, for example, a splice acceptor site (SAS) or a splice donor site (SDS) in exon 13. In some embodiments, the gRNA directs the base editor within 1, 2, 3, 4, 5, 6, 7, 8, 9, 10, 11, 12, 13, 14, 15, 16, 17, 18, 19, or 20 base pairs of a SAS or an SDS within the USH2A gene. In other embodiments, the gRNA directs the base editor within 1, 2, 3, 4, 5, 6, 7, 8, 9, 10, 11, 12, 13, 14, 15, 16, 17, 18, 19, or 20 base pairs of a point mutation within the USH2A gene. In some embodiments, the guide RNA comprises a nucleic acid sequence selected from the group of guide sequences (spacers) consisting of: 5′-CUCUUACCUUGGGAAAGGAG-3′ (SEQ ID NO: 199), 5′-UUCCUUCCCGCAUCAGGGAA-3′ (SEQ ID NO: 200), 5′-GUAGUCAUUAACACAAACUC-3′ (SEQ ID NO: 209), 5′-UUCAAAAGCUGGCCUUAAUG-3′ (SEQ ID NO: 215), 5′-AGACCUCAUGACUCAGUCAA-3′ (SEQ ID NO: 216), 5′-GCUUUAGGGCUUAGGUGUGAU-3′ (SEQ ID NO: 36), 5′-GGAGCUAAAUUACAAUGAAG-3′ (SEQ ID NO: 37), and 5′-CCAUUCAUGCAGGCUACAAG-3′ (SEQ ID NO: 217). Exemplary guide sequences to target USH2A may comprise any of the guide sequences set forth as SEQ ID NOs: 36, 37, and 199-232 and 329. In some embodiments, the gRNA comprises a protospacer sequence that comprises any of the nucleic acid sequences as set forth in SEQ ID NOs: 1-35. The protospacer sequence has the same nucleotide sequence as the spacer sequence of the guide RNA, except the spacer sequence comprises ribonucleotides (e.g., uracils), and the protospacer sequence comprises deoxyribonucleotides (e.g., thymines).

For example, in some embodiments base editing is used to restore US2HA function by disrupting a splice site (or pseudo-splice site) in the USH2A gene sequence to induce skipping of an exon containing a mutation (or a pseudo-exon). This approach is referred to herein as the “exon-skipping approach.” In other embodiments, base editing is used to restore US2HA function by correcting a point mutation (e.g., in an exon) so as to eliminate or reduce mutations. This approach is referred to herein as the “mutation correction approach.”

Further provided herein are compositions and cells comprising complexes of a base editor and a guide RNA. In some embodiments, the napDNAbp domain comprises a Cas9 protein, such as SpCas9, or an ortholog, homolog, or variant thereof (e.g., an SpCas9 nickase), which when complexed with a suitable guide RNA is effective in treating conditions associated with a mutation in the USH2A gene by installing one or more edits (e.g., insertion, deletion, or replacement of one or more nucleobases) in the USH2A genomic locus. Further provided are base-editor-gRNA complexes, and compositions thereof, for use in the preparation of a medicament and for use in treating a subject having or a subject suspected of having Usher syndrome or retinitis pigmentosa (e.g., autosomal recessive RP).

The present disclosure relates in part to the discovery that a variety of base editing strategies to target USH2A genomic loci restores functional Usherin protein, for instance by restoring the normal reading frame of a mutated USH2A gene. Mutations have been observed in USH2A that result in a premature stop codon that results in a truncated USH2A translation product. Mutations in exon 13 account for approximately 35% of all USH2A cases, including a single base deletion at position 2299 (c.2299delG), the most common mutation in USH2A. This mutation creates a frameshift in the coding sequence, resulting in a premature stop codon in exon 13. This premature stop codon is predicted to lead to a null or a truncated, non-functional Usherin protein that causes defective stereocilia formation (ciliogenesis).

The present disclosure further provides vectors, e.g., vectors comprising one or more polynucleotides, encoding a base editor and a guide RNA, or both. In some embodiments, the vectors are recombinant adeno-associated virus (rAAV) vectors. In some embodiments, the vectors are plasmids. rAAV vectors are an efficient and widely used delivery agent that remains the only FDA-approved in vivo gene therapy vector. Further provided herein are kits comprising one or more of these vectors (e.g., rAAV vectors).

In some embodiments involving an adenosine base editor, the step of contacting a target nucleic acid with a base editor results in the deamination of a adenosine nucleobase in the USH2A gene, which results in a T:A base pair in the USH2A gene being converted to a C:G base pair in the USH2A gene (a transition substitution of an “A” to a “G”). In some embodiments, the step of contacting results in deamination of the adenosine nucleobase in the USH2A gene that induces exon skipping which restores (partially or completely) functional Usherin protein. In some embodiments, deaminating the adenosine nucleobase in the USH2A gene corrects a missense mutation or a nonsense mutation that restores functional Usherin protein.

In other embodiments involving a cytidine base editor, the step of contacting a target nucleic acid with a base editor results in the deamination of a cytidine nucleobase in the USH2A gene, which results in a C:G base pair in the USH2A gene being substituted with a T:A base pair in the USH2A gene (a transition substitution of a “C” with a “T”). In some embodiments, the step of contacting results in the deamination of a cytidine nucleobase in the USH2A gene which induces exon skipping that restores (partially or completely) functional Usherin protein. In some embodiments, deaminating the cytidine nucleobase in the USH2A gene corrects a missense mutation or a nonsense mutation that restores functional Usherin protein.

The complexes, polynucleotides, vectors, rAAV particles, and compositions of the disclosure may be formulated for delivery to the inner ear or the eye (e.g., the retina) of a subject. In some embodiments, the subject is diagnosed with, or is suffering from, a disease associated with a deficiency in functional Usherin protein expression. In some embodiments, the subject is diagnosed with, or is suffering from, Usher Syndrome. Accordingly, provided herein are methods of treatment comprising administering one or more of the disclosed polynucleotides, complexes, vectors, rAAV particles, and compositions to a subject, e.g., a human subject suffering from Usher Syndrome. In some embodiments, the subject is diagnosed with, or is suffering from, retinitis pigmentosa (RP). Accordingly, provided herein are methods of treatment comprising administering one or more of the disclosed polynucleotides, complexes, vectors, rAAV particles, and compositions to a subject, e.g., a human subject suffering from RP.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying Figures.

### BRIEF DESCRIPTION OF THE DRAWINGS

The following Figures form part of the present specification and are included to further demonstrate certain aspects of the present disclosure, which can be better understood by reference to one or more of these drawings in combination with the detailed description of specific embodiments presented herein.

FIGS. 1A-1C are schematics that show the common mutations in the USH2A gene. FIG. lA shows the protein domains, in-frame and out-of-frame mutations in exons in the USH2A gene. FIG. 1B shows the unique mutations in each of 25 in-frame exons reported on LOVD (Leiden Open Variation Database), a database of DNA variants indicated as dots labeled with a letter from A to D. FIG. 1C shows the percentage of mutation frequency in each of the 71 exons of the USH2A gene. Mutations in the exon 13 accounts for 31.25% of all Usher syndrome cases.

FIG. 2 is a schematic showing how USH2A mutation candidates are narrowed for base editing treatment. The number of USH2A mutations reported in LOVD is more than 1000. USH2A mutations having an appropriate neighboring PAM sequence for base editors of interest were selected. The editing window was set from position 2 to 13 to select for variants that could be edited in ABE or CBE. The base editor efficacy was tested on twenty-four C:G to T:A mutations (ABEs) and eleven G:C to A:T mutations (CBEs) in USH2A. These mutations are referred to in the drawings and herein as nucleotide sites A1-A24 and C1-C11, respectively.

FIGS. 3A-3B are charts that shows exemplary DNA changes and amino acid substitutions in the USH2A protein that the presently disclosed cytidine base editing methods (at nucleotide sites C1-C11) and adenine base editing methods (at nucleotide sites A1-A24) are designed to correct, along with the pathogenicities associated with these DNA changes. (SEQ ID NOs: 183-193 in FIG. 3A; and SEQ ID NOs: 233-256 in FIG. 3B)

FIGS. 4A-4B are schematics that illustrate show the protocol for the HEK293 knockout experiments. Stable HEK293 cell lines containing the safe harbor transgene were made by using AAVS1-CRISPR system, as shown in FIG. 4A (SEQ ID NO: 1). The target C base is indicated with an asterisk. The base editor (BE) vector and guide RNA (sgRNA) vectors were transfected into the cells and editing efficacy was confirmed by Sanger sequencing, as shown in FIG. 4B.

FIGS. 5A-5C show the base editing efficiency of CBEs on Dnrntl target bases in an AAVS1 stable cell line and mouse retina in vivo. FIG. 5A shows the target DNA sequence (SEQ ID NO: 64). The target C base is indicated with an asterisk. FIG. 5B shows the Sanger sequencing data and indicates that cytosines (C) (indicated with a caret) were converted to thymines (T) in nucleotide site C2, which is at nucleotide position 1841 of the USH2A gene (SEQ ID NOS: 65 (top) and 66 (bottom)). The area was calculated by EditR, a publicly accessible algorithm for predicting potential editing in a guide RNA region from a single Sanger sequencing run. FIG. 5C shows the quantification of values of Sanger sequencing data.

FIGS. 6A-6B show the base editing efficiency in a transfected stable HEK293 cell line. In FIG. 6A, the editing efficiency using CBE (% of conversion) is shown for the 11 CBE target sites. In FIG. 6B, the editing efficiency (% of editing) using ABE7.10 or ABE8 is shown for 24 ABE target sites.

FIGS. 7A-7B show the indel ratio (%) in transfected HEK293 cells. In FIG. 7A, the indel ratio using CBE is shown for 12 different target sites. In FIG. 7B, the indel ratio using ABE7.10 or ABE8 is shown for 24 different target sites.

FIGS. 8A-8C show the confirmation of off-target sequences for nucleotide site C6 which is at nucleotide position 9799 of the USH2A gene. Off-target sites were predicted by CRISPOR, a public software that helps design, evaluate, and clone guide sequences for a CRISPR/Cas9 system. Primers were generated for all off-target sequences, and DNA including target sequence was amplified and analyzed by Sanger sequencing, as shown in the blot of FIG. 8C. This graph shows that targeted editing at C6-OT-3 is fairly efficient, and off-target editing frequency at this site is low. (SEQ ID NOs: 67-73, 194-197 in FIG. 8A).

FIGS. 9A-9C show the confirmation of off-target sequences for nucleotide site A1 (see FIG. 3B). The fourth column indicates the sequence variant nomenclature of the Human Genome Variation Society (HGVS) for each evaluated locus. Off-target sites were predicted by CRISPOR. FIG. 9B shows ABE7.10 and ABE8 data. ABE7.10 edits only OT-5 and ABE8 shows high-efficiency editing. (SEQ ID NOs: 257-266in FIG. 9A). Primers were generated for all off-target sequences, and DNA including target sequence was amplified and analyzed by Sanger sequencing, as shown in the blot of FIG. 9C. The sample lane labels refer to the HGVS labels in FIGS. 9A and 9B as follows: A1-OT2, NC_000002.12G.53254147T>C; A1-OT3, NC_000011.9(0PCM Lv001): c.61+255482A>G; A1-OT4, NC_000001.11(LOC105376673v001): n.138T>C; A1-OT6, NC_000010.11(ATRN L1_v001): n.*599259A>G; A1-OT7, NC_000005. 9: g.124245586T>C; A1-OT9, NC_000021.9(NCAM2_v001): c.55+89989T>C; A1-OT10, NC_000011.91SOX6_v001): c.1251+1418T>C; A1-OT11, NC_000007.13(CDK14_v001): c.*29-38778A>G; A1-OT5, NC_000005.9: g.160332230T>C; AND A1-OT8, NC_000002.12 :g.80229784A>G.

FIGS. 10A-10H show BE-mediated exon skipping of mouse Ush2a exon 12. FIG. 10A is a schematic showing the potential target sites of base editors at splice acceptor site (SAS) and splice donor site (SDS). FIG. 10B shows CBE and ABE base editor and guide sequences (protospacer sequences set forth as SEQ ID NOs: 267-268, respectively) targeting exon 12 SAS and SDS sites. The target base is indicated with an asterisk. Base editing efficiency at the target sites in mouse immortalized organ of Corti k1 (OC-k1) cells and retina. FIG. 10C shows mouse OC-k1 cell and mouse retina transfected with plasmids carrying ABE-xCas9 in one plasmid and EGFP and U6-guides in another plasmid. GFP positive cells (indicated with an arrow) were FACS sorted and used for genomic DNA and RNA. FIG. 10D shows the base correction rate of base editors in OC-k1 cells and mouse retina for the BE4max, ABE7.10, and ABE8e (“ABE8.0”) editors in OC-k1 cells and mouse retina in vivo. FIG. 10E shows the results of Sanger sequencing (SEQ ID NO: 97) and analysis with Edit-R of the PCR-amplified products of DNA isolated from GFP+cells. Primers flanking target site were used. FIG. 1OF shows RT-PCR products of RNA isolated from GFP+OC-k1 cell and mouse retina. Primers flanking mouse exon 11 and 13 were used in the PCR analysis. A ˜290bp band was detected in all editing cells, which matches the expected size if exon 12 is skipped from the transcripts. FIG. 10G shows the results of Sanger sequencing and analysis with Edit-R of of these RT-PCR products, which shows that exon 13 is skipped in frame (SEQ ID NO: 96). FIG. 10H shows the sgRNA sequences of CBE and ABE editors for targeting the splicing acceptor sites of in-frame exons of the USH2A gene (top). These guide sequences are set forth as SEQ ID NOs: 559-568 (from top to bottom). The conversion rates for 10 tested sgRNAs ranges from 20-35% (bottom).

FIG. 11 shows that exon skipping of mouse Ush2a exons in OC-K1 cells results in functional protein. Restoration of ciliogenesis in Ush2a null cells by full length or exon-skipped USH2A protein. Immunostaining of Ush2a (*) and acetylated alpha tubulin (\hat{ }) in wild type, un-transfected Ush2a null cells (clone J) and transfected clone J with mouse full length (Ush2a-FL), human full length (USH2A-FL), and human exon 13, 17, 24, 28, 43, 45, 50, 54, 55, 56, and 62 deleted (USH2A-AEx13, ΔEx17, ΔEx24, ΔEx28, ΔEx43, ΔEx45, AEx50, ΔEx54-55, ΔEx56 and ΔEx62) USH2A cDNA expression plasmids, respectively. Expression of Ush2a and primary cilia marked acetylated alpha tubulin were observed in transfected knockout cells (clone J) cells transfected with ΔEx13, ΔEx17, ΔEx24, ΔEx43, ΔEx45, ΔEx50, and ΔEx56 USH2A cDNA plasmids. No USH2A immunostaining was detected in cells transfected with ΔEx28, ΔEx62, or the negative control ΔEx54-55. Arrows indicate colocalization of Ush2a and alpha tubulin signals. Statistical analysis of the ciliary length in transfected null OC-k1 (clone J) cells from experimental duplicates showed significant differences between non-transfected and transfected Ush2a null cells with 7 USH2A cDNA plasmids. ND: no difference. Each data point represents the cilia length of individual cells (n=100).

FIGS. 12A-12H show the optimization and validation of evaluation systems. FIG. 12A is a schematic that illustrates the protocol for the AAVS1 in vitro knockout in HEK293 cells. FIGS. 12B and 12C are scatter plots that show a comparison of the methods using stable cell lines (pooled cells and single clone), where FIG. 12B shows expression of transgene 1 (TG1) and FIG. 12C shows expression of transgene 2 (TG 2). These plots have been fitted with a line of regression, with the equation shown. A transient expression study was performed by transfecting the same vector including TG1 or TG2 and base editor (BE) vector and sgRNA vector. In parallel, a pooled cell and single clone study was performed by transfecting these cells with BE and sgRNA vectors. FIG. 12D is a scatterplot that shows the comparison of the editing efficiency between the transient expression study and stable single clone study. FIG. 12E is a scatterplot that shows the comparison of editing efficiency between the Sanger analysis and Amplicon sequencing analysis.

FIGS. 13A-13C show the evaluation of the efficacy of base editors on USH2A mutations. (SEQ ID NOS: 269-277 as listed in FIG. 13A; SEQ ID NOs: 393-401, 403 in FIG. 13B; and SEQ ID NOs: 404-409 in FIG.13C).

FIG. 14A is a schematic that illustrates the off-target editing metric “productive editing” (productive edit %), which is calculated as the mean rate at which the contacting of the target sequence with a base editor results in a product that does not contain an amino acid mutation. Nucleotide site A5, which corresponds to nucleotide position 14803, is shown as an example DNA target. (SEQ ID NOs: 164-169 from top to bottom). FIG. 14B shows the editing efficiency (% target conversion) and productive editing % at each mutation of USH2A for the base editors BE3.9, ABE 7.10, and ABE8e.

FIGS. 15A-15D show the restoration of protein expression with base editing of full length USH2A plasmids with mutations. FIG. 15A shows a sequencing trace of A1 (c.11864 G>A) protospacer region (SEQ ID NOS: 410-413 from top to bottom). The black arrow shows USH2A mutation site (Al). The established HEK293 mutant had the mutation “A” in all chromosomes and was transfected with ABE7.10 and ABE8. FIG. 15B shows the editing efficiency was quantified by EditR. FIG. 15C shows immunostaining of USH2A in HEK293 two days after co-transfection with plasmid vector of full length USH2A WT or mutant (sites A1, A7, A12, A15) and base editor/sgRNA plasmid vector. USH2A full length protein is stained by USH2A Ab that recognized intracellular region (encircled in white) and nuclei are stained with Hoechst 33342 (background shading). Bar; 50 μm. FIG. 15D shows cells positive for USH2A full length protein were counted in 6 fields. Data are indicated as mean ±SD (n=6), * p <0.001, by two-tailed Student's t test.

FIGS. 16A-16B show editing with split intein editors. FIG. 16A shows sequencing trace of site Al (c.11864 G>A) protospacer region (SEQ ID NOS: 414 (top) and 415)). The black arrow shows USH2A mutation site (A1). The HEK293 cells were transfected with mutant USH2A plasmids, a guide plasmid, and various editor types. FIG. 16B shows the editing efficiency was quantified by EditR, showing similar editing efficiency with 2 split-intein ABE8 editors.

FIGS. 17A-17B show the editing efficacy of site Al in USH2A using a WT Rhesus monkey. The editing efficacy of site A1 can be estimated by checking the editing efficacy of site A7 in Rhesus monkey sequence because there is no difference in the editing efficacy at sites A3 and A7 between Human mut ABE8 and Rhesus monkey ABE8. (SEQ ID NOs: 416-420 shown from top to bottom in FIG. 17A).

FIG. 18 shows transgene sequences including tandem USH2A mutation sequence and universal primers. Grey highlighted sections show restriction enzyme site, grey-shaded underlined sections show forward primer sequence, and grey-shaded asterisked sections show reverse primer sequences. (SEQ ID NOs: 421 and 422 shown in FIGS. 18A and 18B respectively).

FIG. 19 is a scatterplot showing a comparison of the editing efficacy on Dnmt1 sequence in mouse retina and in stable cell line with transgene. The sequence of 20 bases is the protospacer of Dnmt1, and each potential target “C” is marked with a number.

FIGS. 20A-20B show the editing efficacy of base editor on USH2A mutations by Sanger sequencing. FIG. 20A shows difference in C to T editing efficacy of CBE by three types of Cas9, SpCas9WT (C1, C2, C3, C4, C5 and C6), SpCas9 VQR (C7, C10 and C11) and SaCas9 KKH (C8 and C9). FIG. 20B shows difference in A to G editing efficacy of ABE7.10 and ABE8 by four types of Cas9, SpCas9WT (A1, A5, A15, A18, A20, A22), SpCas9 VQR (A2, A4, A7, A8, A9, A13, A14, A16), SpCas9NG (A11, A12, A17, A19, A21, A23, A24) and SaCas9 KKH (C8 and C9).

FIGS. 21A-21E show NGS analysis of DNA edited by base editor. FIG. 21A shows difference in productive edit, non-productive edit and indel by three types of base editor. The value is mean of each target mutation (CBE3.9; C1-11, ABE7.10; A1-A24, ABE8; A1-A24). FIG. 21B shows a scatter plot editing efficacy vs indel ratio. Each point represents the value of target mutation (CBE3.9:asterisk symbol (*), ABE7.10:plus-sign symbol (+), ABE8: caret symbol (\hat{ })). FIG. 21C shows comparison between the experimental value of editing efficiency and the percentile value as predicted by BE-Hive. FIG. 21D shows a scatter plot showing correlation between experimental ratio of correct sequence and calculated ratio of correct sequence by BE-Hive. The predicted frequency of correct sequence was calculated by inputting the experimental value of editing efficiency into BE-Hive tool. In FIGS. 21C and 21D, the ABE values are indicated with an asterisk.

FIG. 22 is a scatterplot showing relation between Mit off-target score and editing ratio at off-target site.

### DEFINITIONS

As used herein and in the claims, the singular forms “a,” “an,” and “the” include the singular and the plural unless the context clearly indicates otherwise. Thus, for example, a reference to “an agent” includes a single agent and a plurality of such agents.

### AAV

An “adeno-associated virus” or “AAV” is a virus which infects humans and some other primate species. The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10. rAAV particles may comprise a nucleic acid vector (e.g., a recombinant genome), which may comprise at a minimum: (a) one or more heterologous nucleic acid regions comprising a sequence encoding a protein or polypeptide of interest (e.g., a split Cas9 or split nucleobase) or an RNA of interest (e.g., a gRNA), or one or more nucleic acid regions comprising a sequence encoding a Rep protein; and (b) one or more regions comprising inverted terminal repeat (ITR) sequences (e.g., wild-type ITR sequences or engineered ITR sequences) flanking the one or more nucleic acid regions (e.g., heterologous nucleic acid regions). In some embodiments, the nucleic acid vector is between 4 kb and 5 kb in size (e.g., 4.2 to 4.7 kb in size). In some embodiments, the nucleic acid vector further comprises a region encoding a Rep protein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

### Adenosine Deaminase

As used herein, the term “adenosine deaminase” or “adenosine deaminase domain” refers to a protein or enzyme that catalyzes a deamination reaction of an adenosine (or adenine). The terms are used interchangeably. In certain embodiments, the disclosure provides base editors comprising one or more adenosine deaminase domains. For instance, an adenosine deaminase domain may comprise a heterodimer of a first adenosine deaminase and a second deaminase domain, connected by a linker. Adenosine deaminases (e.g., engineered adenosine deaminases or evolved adenosine deaminases) provided herein may be may be enzymes that convert adenine (A) to inosine (I) in DNA or RNA. Such adenosine deaminase can lead to an A:T to G:C base pair conversion. In some embodiments, the deaminase is a variant of a naturally-occurring deaminase from an organism. In some embodiments, the deaminase does not occur in nature. For example, in some embodiments, the deaminase is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

In some embodiments, the adenosine deaminase is derived from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5 ,6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5 ,6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. Reference is made to U.S. Patent Publication No. 2018/0073012, published Mar. 15, 2018, which is incorporated herein by reference.

### Antisense Strand

In genetics, the “antisense” strand of a segment within double-stranded DNA is the template strand, and which is considered to run in the 3′ to 5′ orientation. By contrast, the “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

### Base Editing

“Base editing” refers to genome editing technology that involves the conversion of a specific nucleic acid base into another at a targeted genomic locus. In certain embodiments, this can be achieved without requiring double-stranded DNA breaks (DSB), or single stranded breaks (i.e., nicking). To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g. typically 0.1% to 5%), with the major genome editing products being indels. In order to increase the efficiency of gene correction without simultaneously introducing random indels, the present inventors previously modified the CRISPR/Cas9 system to directly convert one DNA base into another without DSB formation. See, Komor, A. C., et al., Programmable editing of a target base in genomic DNA without double-stranded DNA cleavage. Nature 533, 420-424 (2016), the entire contents of which is incorporated by reference herein.

### Base Editor

The term “base editor (BE),” as used herein, refers to an agent comprising a polypeptide that is capable of making a modification to a base (e.g., A, T, C, G, or U) within a nucleic acid sequence (e.g., DNA or RNA) that converts one base to another (e.g., A to G, AtoC,AtoT,CtoT,CtoG,CtoA,GtoA,GtoC,GtoT,TtoA,TtoC,TtoG). In some embodiments, the base editor is capable of deaminating a base within a nucleic acid such as a base within a DNA molecule. In the case of an adenine base editor, the base editor is capable of deaminating an adenine (A) in DNA. Such base editors may include a nucleic acid programmable DNA binding protein (napDNAbp) fused to an adenosine deaminase. Some base editors include CRISPR-mediated fusion proteins that are utilized in the base editing methods described herein. In some embodiments, the base editor comprises a nuclease-inactive Cas9 (dCas9) fused to a deaminase which binds a nucleic acid in a guide RNA-programmed manner via the formation of an R-loop, but does not cleave the nucleic acid. For example, the dCas9 domain of the fusion protein may include a D10A and a H840A mutation (which renders Cas9 capable of cleaving only one strand of a nucleic acid duplex), as described in PCT/US2016/058344, which published as WO 2017/070632 on Apr. 27, 2017 and is incorporated herein by reference in its entirety. The DNA cleavage domain of S. pyogenes Cas9 includes two subdomains, the HNH nuclease subdomain and the RuvC1 subdomain. The HNH subdomain cleaves the strand complementary to the gRNA (the “targeted strand”, or the strand in which editing or deamination occurs), whereas the RuvC1 subdomain cleaves the non-complementary strand containing the PAM sequence (the “non-edited strand”). The RuvC1 mutant DlOA generates a nick in the targeted strand, while the HNH mutant H840A generates a nick on the non-edited strand (see Jinek et al., Science, 337:816-821(2012); Qi et al., Cell. 28;152(5):1173-83 (2013), each of which are incorporated by reference herein).

In some embodiments, a base editor is a macromolecule or macromolecular complex that results primarily (e.g., more than 80%, more than 85%, more than 90%, more than 95%, more than 99%, more than 99.9%, or 100%) in the conversion of a nucleobase in a polynucleic acid sequence into another nucleobase (i.e., a transition or transversion) using a combination of 1) a nucleotide-, nucleoside-, or nucleobase-modifying enzyme and 2) a nucleic acid binding protein that can be programmed to bind to a specific nucleic acid sequence.

In some embodiments, the base editor comprises a DNA binding domain (e.g., a programmable DNA binding domain such as a dCas9 or nCas9) that directs it to a target sequence. In some embodiments, the base editor comprises a nucleobase modifying enzyme fused to a programmable DNA binding domain (e.g., a dCas9 or nCas9). A “nucleobase modifying enzyme” is an enzyme that can modify a nucleobase and convert one nucleobase to another (e.g., a deaminase such as a cytidine deaminase or a adenosine deaminase). In some embodiments, the base editor may target cytosine (C) bases in a nucleic acid sequence and convert the C to thymine (T) base. In some embodiments, the C to T editing is carried out by a deaminase, e.g., a cytidine deaminase. Base editors that can carry out other types of base conversions (e.g., adenosine (A) to guanine (G), C to G) are also contemplated.

Base editors that convert a C to T, in some embodiments, comprise a cytidine deaminase. A “cytidine deaminase” refers to an enzyme that catalyzes the chemical reaction “cytosine+H₂O→uracil+NH₃” or “5-methyl-cytosine+H₂O→thymine+NH₃.” As it may be apparent from the reaction formula, such chemical reactions result in a C to U/T nucleobase change. In the context of a gene, such a nucleotide change, or mutation, may in turn lead to an amino acid change in the protein, which may affect the protein's function, e.g., loss-of-function or gain-of-function. In some embodiments, the C to T base editor comprises a dCas9 or nCas9 fused to a cytidine deaminase. In some embodiments, the cytidine deaminase domain is fused to the N-terminus of the dCas9 or nCas9. In some embodiments, the base editor further comprises a domain that inhibits uracil glycosylase, and/or a nuclear localization signal. Such base editors have been described in the art, e.g., in Rees & Liu, Nat Rev Genet. 2018; 19(12):770-788 and Koblan et al., Nat Biotechnol. 2018; 36(9):843-846; as well as.U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; International Publication No. WO 2019/023680, published Jan. 31, 2019; International Application No. PCT/US2019/033848, filed May 23, 2019, which published as Publication No. WO 2019/226593 on Nov. 28, 2019; International Publication No. WO 2018/0176009, published Sep. 27, 2018, International Publication No. WO 2020/041751, published Feb. 27, 2020; International Publication No. WO 2020/051360, published Mar. 12, 2020; International Patent Publication No. WO 2020/102659, published May 22, 2020; International Publication No. WO 2020/086908, published Apr. 30, 2020; International Publication No. WO 2020/181180, published Sep. 10, 2020; International Publication No. WO 2020/214842, published Oct. 22, 2020; International Publication No. WO 2020/092453, published May 7, 2020; International Publication No. WO2020/236982, published Nov. 26, 2020; International Application No. PCT/US2020/624628, filed Nov. 25, 2020, and International Application No. PCT/US2021/016827, filed Feb. 5, 2021, the contents of each of which are incorporated herein by reference in their entireties.

In some embodiments, a base editor converts an A to G. In some embodiments, the base editor comprises an adenosine deaminase. An “adenosine deaminase” is an enzyme involved in purine metabolism. It is needed for the breakdown of adenosine from food and for the turnover of nucleic acids in tissues. Its primary function in humans is the development and maintenance of the immune system. An adenosine deaminase catalyzes hydrolytic deamination of adenosine (forming inosine, which base pairs as G) in the context of DNA. There are no known natural adenosine deaminases that act on DNA. Instead, known adenosine deaminase enzymes only act on RNA (tRNA or mRNA). Evolved deoxyadenosine deaminase enzymes that accept DNA substrates and deaminate dA to deoxyinosine have been described, e.g., in PCT Application PCT/US2017/045381, filed Aug. 3, 2017, which published as WO 2018/027078, and PCT Application No. PCT/US2019/033848, filed May 23, 2019, which published on Nov. 28, 2019 as WO 2019/226953, each of which is herein incorporated by reference by reference.

Exemplary adenosine and cytidine base editors are also described in Rees & Liu, Base editing: precision chemistry on the genome and transcriptome of living cells, Nat. Rev. Genet. 2018; 19(12):770-788; as well as U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, the contents of each of which are incorporated herein by reference in their entireties.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casnl nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 domain. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J.A., Charpentier E. Science 337:816-821(2012), the entire contents of which are hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus (e.g., StCas9 or St1Cas9). Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28;152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 74). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 74). In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 74). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 74).

As used herein, the term “nCas9” or “Cas9 nickase” refers to a Cas9 or a variant thereof, which cleaves or nicks only one of the strands of a target cut site thereby introducing a nick in a double strand DNA molecule rather than creating a double strand break. This can be achieved by introducing appropriate mutations in a wild-type Cas9 which inactivates one of the two endonuclease activities of the Cas9. Any suitable mutation which inactivates one Cas9 endonuclease activity but leaves the other intact is contemplated, such as one of D10A or H840A mutations in the wild-type S. pyogenes Cas9 amino acid sequence, or a D10A mutation in the wild-type S. aureus Cas9 amino acid sequence, may be used to form the nCas9.

cDNA

The term “cDNA” refers to a strand of DNA copied from an RNA template. cDNA is complementary to the RNA template.

### Circular Permutant

As used herein, the term “circular permutant” refers to a protein or polypeptide (e.g., a Cas9) comprising a circular permutation, which is change in the protein's structural configuration involving a change in order of amino acids appearing in the protein's amino acid sequence. In other words, circular permutants are proteins that have altered N- and C-termini as compared to a wild-type counterpart, e.g., the wild-type C-terminal half of a protein becomes the new N-terminal half. Circular permutation (or CP) is essentially the topological rearrangement of a protein's primary sequence, connecting its N- and C-terminus, often with a peptide linker, while concurrently splitting its sequence at a different position to create new, adjacent N- and C-termini. The result is a protein structure with different connectivity, but which often can have the same overall similar three-dimensional (3D) shape, and possibly include improved or altered characteristics, including, reduced proteolytic susceptibility, improved catalytic activity, altered substrate or ligand binding, and/or improved thermostability. Circular permutant proteins can occur in nature (e.g., concanavalin A and lectin). In addition, circular permutation can occur as a result of posttranslational modifications or may be engineered using recombinant techniques.

Circularly Permuted napDNAbp

The term “circularly permuted napDNAbp” refers to any napDNAbp protein, or variant thereof (e.g., SpCas9), that occurs as or engineered as a circular permutant, whereby its N- and C-termini have been topically rearranged. Such circularly permuted proteins (“CP-napDNAbp”, such as “CP-Cas9” in the case of Cas9), or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, each of are incorporated herein by reference. The present disclosure contemplates any previously known CP-Cas9 or use a new CP-Cas9 so long as the resulting circularly permuted protein retains the ability to bind DNA when complexed with a guide RNA (gRNA).

### Cytidine Deaminase

As used herein, a “cytidine deaminase” encoded by the CDA gene is an enzyme that catalyzes the removal of an amine group from cytidine (i.e., the base cytosine when attached to a ribose ring) to uridine (C to U) and deoxycytidine to deoxyuridine (C to U). A non-limiting example of a cytidine deaminase is APOBEC1 (“apolipoprotein B mRNA editing enzyme, catalytic polypeptide 1”). Another example is AID (“activation-induced cytidine deaminase”). Under standard Watson-Crick hydrogen bond pairing, a cytosine base hydrogen bonds to a guanine base. When cytidine is converted to uridine (or deoxycytidine is converted to deoxyuridine), the uridine (or the uracil base of uridine) undergoes hydrogen bond pairing with the base adenine. Thus, a conversion of “C” to uridine (“U”) by cytidine deaminase will cause the insertion of “A” instead of a “G” during cellular repair and/or replication processes. Since the adenine “A” pairs with thymine “T”, the cytidine deaminase in coordination with DNA replication causes the conversion of an C G pairing to a T A pairing in the double-stranded DNA molecule.

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species—the guide RNA. See, e.g., Jinek M., Chylinski K., Fonfara I., Hauer M., Doudna J.A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. therrnophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

### Deaminase

The term “deaminase” or “deaminase domain” refers to a protein or enzyme that catalyzes a deamination reaction. In some embodiments, the deaminase is an adenosine (or adenine) deaminase, which catalyzes the hydrolytic deamination of adenine or adenosine. In some embodiments, the adenosine deaminase catalyzes the hydrolytic deamination of adenine or adenosine in deoxyribonucleic acid (DNA) to inosine. In other embodiments, the deminase is a cytidine (or cytosine) deaminase, which catalyzes the hydrolytic deamination of cytidine or cytosine.

The deaminases described herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

### DNA Binding Protein

As used herein, the term “DNA binding protein” or “DNA binding protein domain” refers to any protein that localizes to and binds a specific target DNA nucleotide sequence (e.g. a gene locus of a genome). This term embraces RNA-programmable proteins, which associate (e.g. form a complex) with one or more nucleic acid molecules (i.e., which includes, for example, guide RNA in the case of Cas systems) that direct or otherwise program the protein to localize to a specific target nucleotide sequence (e.g., DNA sequence) that is complementary to the one or more nucleic acid molecules (or a portion or region thereof) associated with the protein. Exemplary RNA-programmable proteins are CRISPR-Cas9 proteins, as well as Cas9 equivalents, homologs, orthologs, or paralogs, whether naturally occurring or non-naturally occurring (e.g. engineered or modified), and may include a Cas9 equivalent from any type of CRISPR system (e.g. type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, (e.g., LbCas12a, AsCas12a, CeCas12a and MbCas12a), Cas12b, Cas12c, Cas12d, Cas12g, Cas12h, Cas12i, Cas13d, Cas14, Argonaute (Ago), nCas9, xCas9, SpCas9-NG, SaCas9-KKH, SmacCas9, Spy-macCas9, SpCas9-NRRH, SpCas9-NRCH, SpCas9-NRTH, SpCas9-VQR, and circularly permuted Cas9 domains, such as CP1012, CP1028, CP1041, CP1249, and CP1300. Additional napDNAbp Cas equivalents include Cas3 and Cas(I). Additional Cas-equivalents are described in Makarova et al., “C2c2 is a single-component programmable RNA-guided RNA-targeting CRISPR effector,” Science 2016; 353(6299), the contents of which are incorporated herein by reference.

### DNA Editing Efficiency

The term “DNA editing efficiency,” as used herein, refers to the number or proportion of intended base pairs that are edited. For example, if a base editor edits 10% of the base pairs that it is intended to target (e.g., within a cell or within a population of cells), then the base editor can be described as being 10% efficient. Some aspects of editing efficiency embrace the modification (e.g. deamination) of a specific nucleotide within DNA, without generating a large number or percentage of insertions or deletions (i.e., indels). It is generally accepted that editing while generating less than 5% indels (as measured over total target nucleotide substrates) is high editing efficiency. The generation of more than 20% indels is generally accepted as poor or low editing efficiency. Indel formation may be measured by techniques known in the art, including high-throughput screening of sequencing reads.

The term “off-target editing frequency,” as used herein, refers to the number or proportion of unintended base pairs, e.g. DNA base pairs, that are edited. On-target and off-target editing frequencies may be measured by the methods and assays described herein, further in view of techniques known in the art, including high-throughput sequencing reads. As used herein, high-throughput sequencing involves the hybridization of nucleic acid primers (e.g., DNA primers) with complementarity to nucleic acid (e.g., DNA) regions just upstream or downstream of the target sequence or off-target sequence of interest. Because the DNA target sequence and the Cas9-independent off-target sequences are known a priori in the methods disclosed herein, nucleic acid primers with sufficient complementarity to regions upstream or downstream of the target sequence and Cas9-independent off-target sequences of interest may be designed using techniques known in the art, such as the PhusionU PCR kit (Life Technologies), Phusion HS II kit (Life Technologies), and Illumina MiSeq kit. The number of off-target DNA edits may be measured by techniques known in the art, including high-throughput screening of sequencing reads, EndoV-Seq, GUIDE-Seq, CIRCLE-Seq, and Cas-OFFinder. Since many of the Cas9-dependent off-target sites have high sequence identity to the target site of interest, nucleic acid primers with sufficient complementarity to regions upstream or downstream of the Cas9-dependent off-target site may likewise be designed using techniques and kits known in the art. These kits make use of polymerase chain reaction (PCR) amplification, which produces amplicons as intermediate products. The target and off-target sequences may comprise genomic loci that further comprise protospacers and PAMs. Accordingly, the term “amplicons,” as used herein, may refer to nucleic acid molecules that constitute the aggregates of genomic loci, protospacers and PAMs. High-throughput sequencing techniques used herein may further include Sanger sequencing and Illumina-based next-generation genome sequencing (NGS).

The term “on-target editing,” as used herein, refers to the introduction of intended modifications (e.g., deaminations) to nucleotides (e.g., adenine) in a target sequence, such as using the base editors described herein. The term “off-target DNA editing,” as used herein, refers to the introduction of unintended modifications (e.g. deaminations) to nucleotides (e.g. adenine) in a sequence outside the canonical base editor binding window (i.e., from one protospacer position to another, typically 2 to 8 nucleotides long). Off-target DNA editing can result from weak or non-specific binding of the gRNA sequence to the target sequence. As used herein, the term “bystander editing” refers to synonymous off-target point mutations at nucleobases that are near (proximate to) the target base and do not change the outcome of the intended editing method.

As used herein, the terms “purity” and “product purity” of a base editor refer to the mean the percentage of edited sequencing reads (reads in which the target nucleobase has been converted to a different base) in which the intended conversion occurs (e.g., for a cytosine base editor, in which the target C is edited to a T). See Komor et al., Sci Adv 3 (2017).

### Downstream

As used herein, the terms “upstream” and “downstream” are terms of relativety that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3 ′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

### Effective Amount

The term “effective amount,” as used herein, refers to an amount of a biologically active agent that is sufficient to elicit a desired biological response. For example, in some embodiments, an effective amount of a base editor may refer to the amount of the editor that is sufficient to edit a target site nucleotide sequence, e.g., a genome. In some embodiments, an effective amount of a base editor described herein, e.g., of a base editor comprising a nickase Cas9 domain and a guide RNA may refer to the amount of the base editor that is sufficient to induce editing of a target site specifically bound and edited by the base editor. As will be appreciated by the skilled artisan, the effective amount of an agent, e.g., a base editor, a nuclease, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

### Functional Equivalent

The term “functional equivalent” refers to a second biomolecule that is equivalent in function, but not necessarily equivalent in structure to a first biomolecule. For example, a “Cas9 equivalent” refers to a protein that has the same or substantially the same functions as Cas9, but not necessarily the same amino acid sequence. In the context of the disclosure, the specification refers throughout to “a protein X, or a functional equivalent thereof.” In this context, a “functional equivalent” of protein X embraces any homolog, paralog, fragment, naturally occurring, engineered, circular permutant, mutated, or synthetic version of protein X which bears an equivalent function.

### Fusion Protein

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Another example includes a Cas9 or equivalent thereof fused to an adenosine deaminae. Any of the proteins described herein may be produced by any method known in the art. For example, the proteins described herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

### Guide Nucleic Acid

The term “guide nucleic acid” or “napDNAbp-programming nucleic acid molecule” or equivalently “guide sequence” refers the one or more nucleic acid molecules which associate with and direct or otherwise program a napDNAbp protein to localize to a specific target nucleotide sequence (e.g., a gene locus of a genome) that is complementary to the one or more nucleic acid molecules (or a portion or region thereof) associated with the protein, thereby causing the napDNAbp protein to bind to the nucleotide sequence at the specific target site. A non-limiting example is a guide RNA of a Cas protein of a CRISPR-Cas genome editing system. Chemically, guide nucleic acids can be all RNA, all DNA, or a chimeric of RNA and DNA. The guide nucleic acids may also include nucleotide analogs. Guide nucleic acids can be expressed as transcription products or can be synthesized.

Guide RNA (“gRNA”)

As used herein, a “guide RNA” can refer to a synthetic fusion of the endogenous bacterial crRNA and tracrRNA that provides both targeting specificity and a scaffold and/or binding ability for Cas9 nuclease to a target DNA. This synthetic fusion does not exist in nature and is also commonly referred to as an sgRNA. However, the term, guide RNA, also embraces equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein. In addition, methods for designing appropriate guide RNA sequences are provided herein.

A guide RNA is a particular type of guide nucleic acid which is mostly commonly associated with a Cas protein of a CRISPR-Cas9 and which associates with Cas9, directing the Cas9 protein to a specific sequence in a DNA molecule that includes complementarity to the protospacer sequence for the guide RNA. Functionally, guide RNAs associate with Cas9, directing (or programming) the Cas9 protein to a specific sequence in a DNA molecule that includes a sequence complementary to the protospacer sequence for the guide RNA.

As used herein, a “spacer sequence” is the sequence of the guide RNA (-20 nts in length) which has the same sequence (with the exception of uridine bases in place of thymine bases) as the protospacer of the PAM strand of the target (DNA) sequence, and which is complementary to the target strand (or non-PAM strand) of the target sequence.

### Guide RNA Target Sequence

As used herein, the “target sequence” refers to the ˜20 nucleotides in the target DNA sequence that have complementarity to the protospacer sequence in the PAM strand. The target sequence is the sequence that anneals to or is targeted by the spacer sequence of the guide RNA. The spacer sequence of the guide RNA and the protospacer have the same sequence (except the spacer sequence is RNA, and the protospacer is DNA).

### Guide RNA Scaffold Sequence

As used herein, the terms “guide RNA core,” “guide RNA scaffold sequence” and “backbone sequence” refer to the sequence within the gRNA that is responsible for Cas9 binding, it does not include the 20 bp spacer sequence that is used to guide Cas9 to target DNA.

### Host Cell

The term “host cell,” as used herein, refers to a cell that can host and replicate a vector encoding a base editor, guide RNA, and/or combination thereof, as described herein. In some embodiments, host cells are mammalian cells, such as human cells. Provided herein are methods of transducing and transfecting a host cell, such as a human cell, e.g., a human cell in a subject, with one or more vectors provided herein, such as one or more viral (e.g., rAAV) vectors provided herein.

It should be appreciated that any of the base editors, guide RNAs, and or combinations thereof, described herein may be introduced into a host cell in any suitable way, either stably or transiently. In some embodiments, a base editor may be transfected into the host cell. In some embodiments, the host cell may be transduced or transfected with a nucleic acid construct that encodes a base editor. For example, a host cell may be transduced (e.g., with a viral particle encoding a base editor) with a nucleic acid that encodes a base editor, or the translated base editor. As an additional example, a host cell may be transfected with a nucleic acid (e.g., a plasmid) that encodes a base editor or the translated base editor. Such transductions or transfections may be stable or transient. In some embodiments, host cells expressing a base editor or containing a base editor may be transduced or transfected with one or more gRNA molecules, for example when the base editor comprises a Cas9 (e.g., nCas9) domain. In some embodiments, a plasmid expressing a base editor may be introduced into host cells through electroporation, transient transfection (e.g., lipofection, such as with Lipofectamine 3000®), stable genome integration (e.g., piggybac), viral transduction, or other methods known to those of skill in the art.

Also provided herein are host cells for packaging of viral particles. In embodiments where the vector is a viral vector, a suitable host cell is a cell that may be infected by the viral vector, can replicate it, and can package it into viral particles that can infect fresh host cells. A cell can host a viral vector if it supports expression of genes of viral vector, replication of the viral genome, and/or the generation of viral particles. In some embodiments, the host cell is a eukaryotic cell, for example, a yeast cell, an insect cell, or a mammalian cell. The type of host cell, will, of course, depend on the vector employed, and suitable host cell/vector combinations will be readily apparent to those of skill in the art.

### Inteins and Split-Inteins

As used herein, the term “intein” refers to auto-processing polypeptide domains found in organisms from all domains of life. An intein (intervening protein) carries out a unique auto-processing event known as protein splicing in which it excises itself out from a larger precursor polypeptide through the cleavage of two peptide bonds and, in the process, ligates the flanking extein (external protein) sequences through the formation of a new peptide bond. This rearrangement occurs post-translationally (or possibly co-translationally), as intein genes are found embedded in frame within other protein-coding genes. Furthermore, intein-mediated protein splicing is spontaneous; it requires no external factor or energy source, only the folding of the intein domain. This process is also known as cis-protein splicing, as opposed to the natural process of trans-protein splicing with “split inteins.”

Split inteins are a sub-category of inteins. Unlike the more common contiguous inteins, split inteins are transcribed and translated as two separate polypeptides, the N-intein and C-intein, each fused to one extein. Upon translation, the intein fragments spontaneously and non-covalently assemble into the canonical intein structure to carry out protein splicing in trans.

Inteins and split inteins are the protein equivalent of the self-splicing RNA introns (see Perler et al., Nucleic Acids Res. 22:1125-1127 (1994)), which catalyze their own excision from a precursor protein with the concomitant fusion of the flanking protein sequences, known as exteins (reviewed in Perler et al., Curr. Opin. Chem. Biol. 1:292-299 (1997); Perler, F. B. Cell 92(1):1-4 (1998); Xu et al., EMBO J. 15(19):5146-5153 (1996)).

As used herein, the term “protein splicing” refers to a process in which an interior region of a precursor protein (an intein) is excised and the flanking regions of the protein (exteins) are ligated to form the mature protein. This natural process has been observed in numerous proteins from both prokaryotes and eukaryotes (Perler, F. B., Xu, M. Q., Paulus, H. Current Opinion in Chemical Biology 1997, 1, 292-299; Perler, F. B. Nucleic Acids Research 1999, 27, 346-347). The intein unit contains the necessary components needed to catalyze protein splicing and often contains an endonuclease domain that participates in intein mobility (Perler, F. B., Davis, E. 0., Dean, G. E., Gimble, F. S., Jack, W. E., Neff, N., Noren, C. J., Thomer, J., Belfort, M. Nucleic Acids Research 1994, 22, 1127-1127). The resulting proteins are linked, however, not expressed as separate proteins. Protein splicing may also be conducted in trans with split inteins expressed on separate polypeptides spontaneously combine to form a single intein which then undergoes the protein splicing process to join to separate proteins.

The elucidation of the mechanism of protein splicing has led to a number of intein-based applications (Comb, et al., U.S. Pat. No. 5,496,714; Comb, et al., U.S. Pat. No. 5,834,247; Camarero and Muir, J. Amer. Chem. Soc., 121:5597-5598 (1999); Chong, et al., Gene, 192:271-281 (1997), Chong, et al., Nucleic Acids Res., 26:5109-5115 (1998); Chong, et al., J. Biol. Chem., 273:10567-10577 (1998); Cotton, et al. J. Am. Chem. Soc., 121:1100-1101 (1999); Evans, et al., J. Biol. Chem., 274:18359-18363 (1999); Evans, et al., J. Biol. Chem., 274:3923-3926 (1999); Evans, et al., Protein Sci., 7:2256-2264 (1998); Evans, et al., J. Biol. Chem., 275:9091-9094 (2000); Iwai and Pluckthun, FEBS Lett. 459:166-172 (1999); Mathys, et al., Gene, 231:1-13 (1999); Mills, et al., Proc. Natl. Acad. Sci. USA 95:3543-3548 (1998); Muir, et al., Proc. Natl. Acad. Sci. USA 95:6705-6710 (1998); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999); Severinov and Muir, J. Biol. Chem., 273:16205-16209 (1998); Shingledecker, et al., Gene, 207:187-195 (1998); Southworth, et al., EMBO J. 17:918-926 (1998); Southworth, et al., Biotechniques, 27:110-120 (1999); Wood, et al., Nat. Biotechnol., 17:889-892 (1999); Wu, et al., Proc. Natl. Acad. Sci. USA 95:9226-9231 (1998a); Wu, et al., Biochim Biophys Acta 1387:422-432 (1998b); Xu, et al., Proc. Natl. Acad. Sci. USA 96:388-393 (1999); Yamazaki, et al., J. Am. Chem. Soc., 120:5591-5592 (1998)). Each reference is incorporated herein by reference.

### Linker

The term “linker,” as used herein, refers to a chemical group or a molecule linking two molecules or domains, e.g. dCas9 and a deaminase. Typically, the linker is positioned between, or flanked by, two groups, molecules, or other domains and connected to each one via a covalent bond, thus connecting the two. In some embodiments, the linker is an amino acid or a plurality of amino acids (e.g. a peptide or protein). In some embodiments, the linker is an organic molecule, group, polymer, or chemical domain. Chemical groups include, but are not limited to, disulfide, hydrazone, and azide domains. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, the linker is an XTEN linker. In some embodiments, the linker is a 32-amino acid linker. In other embodiments, the linker is a 30-, 31-, 33- or 34-amino acid linker.

### Mutation

The term “mutation,” as used herein, refers to a substitution of a residue within a sequence, e.g. a nucleic acid or amino acid sequence, with another residue; a deletion or insertion of one or more residues within a sequence; or a substitution of a residue within a sequence of a genome in a subject to be corrected. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which are mutations that reduce or abolish a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome, which results from a mutation in the gene for the connective tissue protein called fibrillin. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. Because of their nature, gain-of-function mutations are usually dominant. Many loss-of-function mutations are recessive, such as autosomal recessive. Many of the USH2A mutations for which the presently disclosed base editing methods aim to correct are autosomal recessive.

napDNAbp

The term “napDNAbp” which stand for “nucleic acid programmable DNA binding protein” refers to any protein that may associate (e.g., form a complex) with one or more nucleic acid molecules (i.e., which may broadly be referred to as a “napDNAbp-programming nucleic acid molecule” and includes, for example, guide RNA in the case of Cas systems) which direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. This term napDNAbp embraces CRISPR-Cas9 proteins, as well as Cas9 equivalents, homologs, orthologs, or paralogs, whether naturally occurring or non-naturally occurring (e.g., engineered or modified), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353 (6299), the contents of which are incorporated herein by reference. However, the nucleic acid programmable DNA binding protein (napDNAbp) that may be used in connection with this invention are not limited to CRISPR-Cas systems. The invention embraces any such programmable protein, such as the Argonaute protein from Natronobacteriurn gregoryi (NgAgo) which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao et al., DNA-guided genome editing using the Natronobacterium gregoryi Argonaute. Nature Biotechnology 2016; 34(7):768-73, which is incorporated herein by reference.

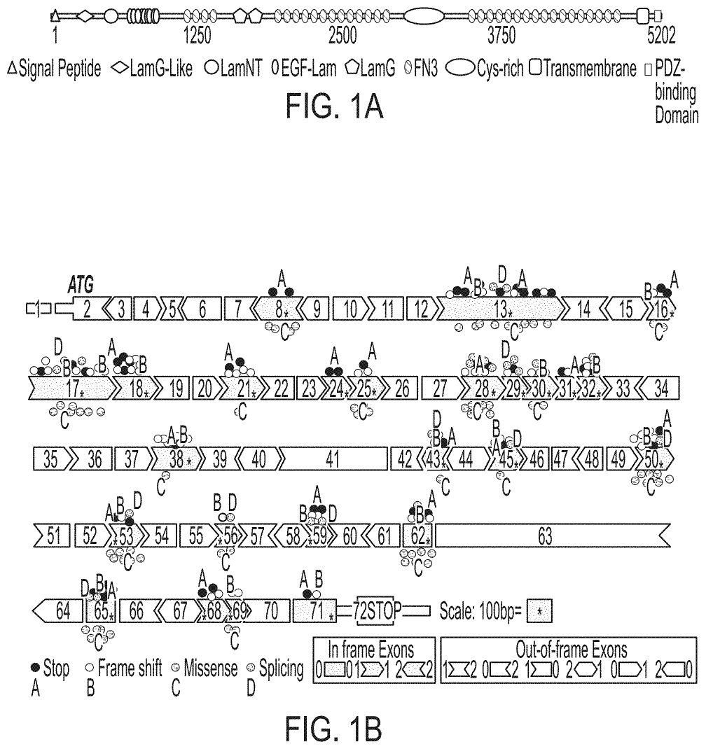

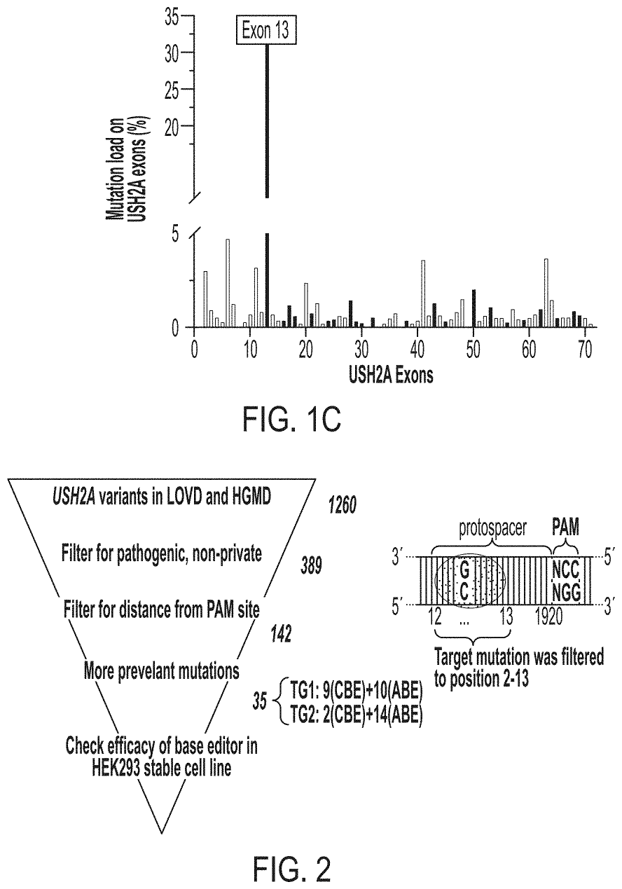

In some embodiments, the napDNAbp is a RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821(2012), the entire contents of which is incorporated herein by reference. Other examples of gRNAs (e.g., those including domain 2) can be found in U.S. Pat. No. 9,340,799, entitled “mRNA-Sensing Switchable gRNAs,” and International Patent Application No. PCT/US2014/054247, filed Sep. 6, 2013, published as WO 2015/035136 and entitled “Delivery System For Functional Nucleases,” the entire contents of each are herein incorporated by reference. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csnl) from Streptococcus pyogenes (see, e.g., “Complete genome sequence of an M1 strain of Streptococcus pyogenes.” Ferretti J. J. et al., Proc. Natl. Acad. Sci. U.S.A. 98:4658-4663(2001); “CRISPR RNA maturation by trans-encoded small RNA and host factor RNase III.” Deltcheva E. et al., Nature 471:602-607(2011); and “A programmable dual-RNA-guided DNA endonuclease in adaptive bacterial immunity.” Jinek M. et al., Science 337:816-821(2012), the entire contents of each of which are incorporated herein by reference. The napDNAbp nucleases (e.g., Cas9) use RNA:DNA hybridization to target DNA cleavage sites, these proteins are able to be targeted, in principle, to any sequence specified by the guide RNA. Methods of using napDNAbp nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo, J. E. et al., Genome engineering in Saccharomyces cerevisiae using CRISPR-Cas systems. Nucleic Acid Res. (2013); Jiang, W. et al. RNA-guided editing of bacterial genomes using CRISPR-Cas systems. Nature Biotechnology 31, 233-239 (2013); the entire contents of each of which are incorporated herein by reference).

### Nickase

The term “nickase” refers to a napDNAbp (e.g., a Cas9) having only a single nuclease activity that cuts only one strand of a target DNA, rather than both strands. Thus, a nickase type napDNAbp does not leave a double-strand break. Exemplary nickases include SpCas9 and SaCas9 nickases. An exemplary nickase comprises a sequence having at least 99%, or 100%, identity to the amino acid sequence of SEQ ID NO: 107.

### Nuclear Localization Signal

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences may be of any size and composition, for example, more than 25, 25, 15, 12, 10, 8, 7, 6, 5, or 4 amino acids, but will preferably comprise at least a four to eight amino acid sequence known to function as a nuclear localization signal (NLS).

### Nucleic Acid Molecule

The term “nucleic acid molecule” as used herein, refers to RNA as well as single and/or double-stranded DNA. Nucleic acid molecules may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g. a recombinant DNA or RNA, an artificial chromosome, an engineered genome, or fragment thereof, or a synthetic DNA, RNA, DNA/RNA hybrid, or including non-naturally occurring nucleotides or nucleosides.

Furthermore, the terms “nucleic acid,” “DNA,” “RNA,” and/or similar terms include nucleic acid analogs, e.g. analogs having other than a phosphodiester backbone. Nucleic acids may be purified from natural sources, produced using recombinant expression systems and optionally purified, chemically synthesized, etc. Where appropriate, e.g. in the case of chemically synthesized molecules, nucleic acids may comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g. 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, inosinedenosine, 8-oxoguanosine, O(6)-methylguanine, and 2-thiocytidine); chemically modified bases; biologically modified bases (e.g., methylated bases, such as 2′-O-methylated bases); intercalated bases; modified sugars (e.g. 2′-fluororibose, ribose, 2′-deoxyribose, arabinose, and hexose); and/or modified phosphate groups (e.g. phosphorothioates and 5′-N-phosphoramidite linkages).

### PACE

The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference.

### Promoter

The term “promoter” is art-recognized and refers to a nucleic acid molecule with a sequence recognized by the cellular transcription machinery and able to initiate transcription of a downstream gene. A promoter may be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters is inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect. In various embodiments, the disclosure provides vectors with appropriate promoters for driving expression of the nucleic acid sequences encoding the base editors (or one or more individual components thereof).

### Protospacer

As used herein, the term “protospacer” refers to the sequence (e.g., a ˜20 bp sequence) in DNA adjacent to the PAM (protospacer adjacent motif) sequence which shares the same sequence as the spacer sequence of the guide RNA, and which is complementary to the target sequence of the non-PAM strand. The spacer sequence of the guide RNA anneals to the target sequence located on the non-PAM strand. In order for Cas9 to function it also requires a specific protospacer adjacent motif (PAM) that varies depending on the bacterial species of the Cas9 gene. The most commonly used Cas9 nuclease, derived from S. pyogenes, recognizes a PAM sequence of NGG that is found directly downstream of the protospacer sequence in the genomic DNA, on the non-target strand. The skilled person will appreciate that the literature in the state of the art sometimes refers to the “protospacer” as the ˜20-nt target-specific guide sequence on the guide RNA itself, rather than referring to it as a “spacer” (and that the protospacer (DNA) and the spacer (RNA) have the same sequence). Thus, the term “protospacer” as used herein may be used interchangeably with the term “spacer.” The context of the discription surrounding the appearance of either “protospacer” or “spacer” will help inform the reader as to whether the term is refence to the gRNA or the DNA sequence. Both usages of these terms are acceptable since the state of the art uses both terms in each of these ways.

### Protospacer Adjacent Motif (PAM)

As used herein, the term “protospacer adjacent sequence” or “PAM” refers to an approximately 2-6 base pair DNA sequence that is an important targeting component of a Cas9 nuclease. Typically, the PAM sequence is on either strand, and is downstream in the 5′ to 3′ direction of Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes alternative PAM sequence.

For example, with reference to the canonical SpCas9 amino acid sequence is SEQ ID NO: 74, the PAM sequence can be modified by introducing one or more mutations, including (a) D1135V, R1335Q, and T1337R “the VRQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These are example are not meant to be limiting. It will be further appreciated that non-SpCas9s bind a variety of PAM sequences, which makes them useful when no suitable SpCas9 PAM sequence is present at the desired target cut site. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10(5): 891-899 (which is incorporated herein by reference).

### Protein, Peptide, and Polypeptide

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. It should be appreciated that the disclosure provides any of the polypeptide sequences provided herein without an N-terminal methionine (M) residue.

### Sense Strand

In genetics, a “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. In some embodiments, the subject is a domesticated animal. In some embodiments, the subject is a plant.

### Target Site

The term “target site” refers to a sequence within a nucleic acid molecule that is edited by a base editor (BE) disclosed herein. The term “target site,” in the context of a single strand, also can refer to the “target strand” which anneals or binds to the spacer sequence of the guide RNA. The target site can refer, in certain embodiments, to a segment of double-stranded DNA that includes the protospacer (i.e., the strand of the target site that has the same nucleotide sequence as the spacer sequence of the guide RNA) on the PAM-strand (or non-target strand) and target strand, which is complementary to the protospacer and the spacer alike, and which anneals to the spacer of the guide RNA, thereby targeting or programming a Cas9 base editor to target the target site.

### Transcriptional Terminator

A “transcriptional terminator” is a nucleic acid sequence that causes transcription to stop. A transcriptional terminator may be unidirectional or bidirectional. It is comprised of a DNA sequence involved in specific termination of an RNA transcript by an RNA polymerase. A transcriptional terminator sequence prevents transcriptional activation of downstream nucleic acid sequences by upstream promoters. A transcriptional terminator may be necessary in vivo to achieve desirable expression levels or to avoid transcription of certain sequences. A transcriptional terminator is considered to be “operably linked to” a nucleotide sequence when it is able to terminate the transcription of the sequence it is linked to. The most commonly used type of terminator is a forward terminator. When placed downstream of a nucleic acid sequence that is usually transcribed, a forward transcriptional terminator will cause transcription to abort. In some embodiments, bidirectional transcriptional terminators are provided, which usually cause transcription to terminate on both the forward and reverse strand. In some embodiments, reverse transcriptional terminators are provided, which usually terminate transcription on the reverse strand only.

In prokaryotic systems, terminators usually fall into two categories (1) rho-independent terminators and (2) rho-dependent terminators. Rho-independent terminators are generally composed of palindromic sequence that forms a stem loop rich in G-C base pairs followed by several T bases. Without wishing to be bound by theory, the conventional model of transcriptional termination is that the stem loop causes RNA polymerase to pause, and transcription of the poly-A tail causes the RNA:DNA duplex to unwind and dissociate from RNA polymerase.

In eukaryotic systems, the terminator region may comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in some embodiments involving eukaryotes, a terminator may comprise a signal for the cleavage of the RNA. In some embodiments, the terminator signal promotes polyadenylation of the message. The terminator and/or polyadenylation site elements may serve to enhance output nucleic acid levels and/or to minimize read through between nucleic acids.

Terminators for use in accordance with the present disclosure include any terminator of transcription described herein or known to one of ordinary skill in the art. Examples of terminators include, without limitation, the termination sequences of genes such as, for example, the bovine growth hormone terminator, and viral termination sequences such as, for example, the SV40 terminator, spy, yejM, secG-leuU, thrLABC, rrnB Ti, hisLGDCBHAFI, metZWV, rrnC, xapR, aspA and arcA terminator. In some embodiments, the termination signal may be a sequence that cannot be transcribed or translated, such as those resulting from a sequence truncation.

### Transition

As used herein, “transitions” refer to the interchange of purine nucleobases (A↔G) or the interchange of pyrimidine nucleobases (C↔T). This class of interchanges involves nucleobases of similar shape. The compositions and methods disclosed herein are capable of inducing one or more transitions in a target DNA molecule. The compositions and methods disclosed herein are also capable of inducing both transitions and transversion in the same target DNA molecule. These changes involve A↔G, G↔A, C↔T, or T↔C. In the context of a double-strand DNA with Watson-Crick paired nucleobases, transitions refer to the following base pair exchanges: A:T↔G:C, G:G↔A:T, C:G↔T:A, or T:A↔C:G. The compositions and methods disclosed herein are capable of inducing one or more transitions in a target DNA molecule. The compositions and methods disclosed herein are also capable of inducing both transitions and transversion in the same target DNA molecule, as well as other nucleotide changes, including deletions and insertions.

### Transversion

As used herein, “transversions” refer to the interchange of purine nucleobases for pyrimidine nucleobases, or in the reverse and thus, involve the interchange of nucleobases with dissimilar shape. These changes involve T↔A, T4->G, C↔G, C↔A, A↔T, A↔C, G↔C, and G↔T. In the context of a double-strand DNA with Watson-Crick paired nucleobases, transversions refer to the following base pair exchanges: T:A↔A:T, T:A↔G:C, C:G↔G:C, C:G↔A:T, A:T↔T:A, A:T↔C:G, G:C↔C:G, and G:C↔T:A. The compositions and methods disclosed herein are capable of inducing one or more transversions in a target DNA molecule. The compositions and methods disclosed herein are also capable of inducing both transitions and transversion in the same target DNA molecule, as well as other nucleotide changes, including deletions and insertions.

### Treatment

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Upstream

As used herein, the terms “upstream” and “downstream” are terms of relativety that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

### Uracil Glycosylase Inhibitor

The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 41. In some embodiments, the UGI proteins described herein include fragments of UGI and proteins homologous to a UGI or a UGI fragment. For example, in some embodiments, a UGI domain comprises a fragment of the amino acid sequence set forth in SEQ ID NO: 41. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 41. In some embodiments, a UGI comprises an amino acid sequence homologous to the amino acid sequence set forth in SEQ ID NO: 41, or an amino acid sequence homologous to a fragment of the amino acid sequence set forth in SEQ ID NO: 41. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 41. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 41. In some embodiments, the UGI comprises the following amino acid sequence: MTNLSDIIEKETGKQLVIQESILMLPEEVEEVIGNKPESDILVHTAYDESTDENVMLLT SDAPEYKPWALVIQDSNGENKIKML (SEQ ID NO: 41) (P14739IUNGIBPPB2 Uracil-DNA glycosylase inhibitor).

### Variant

As used herein, the term “variant” refers to a protein having characteristics that deviate from what occurs in nature that retains at least one functional i.e. binding, interaction, or enzymatic ability and/or therapeutic property thereof. A “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant of Cas9 may comprise a Cas9 that has one or more changes in amino acid residues as compared to a wild type Cas9 amino acid sequence. As another example, a variant of a deaminase may comprise a deaminase that has one or more changes in amino acid residues as compared to a wild type deaminase amino acid sequence, e.g. following ancestral sequence reconstruction of the deaminase. These changes include chemical modifications, including substitutions of different amino acid residues truncations, covalent additions (e.g. of a tag), and any other mutations. The term also encompasses circular permutants, mutants, truncations, or domains of a reference sequence, and which display the same or substantially the same functional activity or activities as the reference sequence. This term also embraces fragments of a wild type protein.

The level or degree of which the property is retained may be reduced relative to the wild type protein but is typically the same or similar in kind. Generally, variants are overall very similar, and in many regions, identical to the amino acid sequence of the protein described herein. A skilled artisan will appreciate how to make and use variants that maintain all, or at least some, of a functional ability or property.

The variant proteins may comprise, or alternatively consist of, an amino acid sequence which is at least 80%, 85%, 90%, 95%, 96%, 97%, 98%, 99%, or 100%, identical to, for example, the amino acid sequence of a wild-type protein, or any protein provided herein.

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence of a protein such as a Usherin protein, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

### Vector

The term “vector,” as used herein, refers to a nucleic acid that can be modified to encode a gene of interest and that is able to enter into a host cell and replicate within the host cell, and then transfer a replicated form of the vector into another host cell. Exemplary suitable vectors include viral vectors, such as AAV vectors or bacteriophages and filamentous phage, and conjugative plasmids. Additional suitable vectors will be apparent to those of skill in the art based on the present disclosure.

### Wild Type

As used herein the term “wild type” is a term of the art understood by skilled persons and means the typical form of an organism, strain, gene or characteristic as it occurs in nature as distinguished from mutant or variant forms.

These and other exemplary substituents are described in more detail in the Detailed Description, Examples, and claims. The invention is not intended to be limited in any manner by the above exemplary listing of substituents.

### Detailed Description of Certain Embodiments

The present disclosure provides compositions, vectors, kits, and methods for modifying a polynucleotide using base editing strategies that comprise the use of a nucleic acid programmable DNA binding protein (“napDNAbp”), a deaminase (e.g., cytidine or adenosine deaminase) and a suitable guide RNA that targets the USH2A gene in order to treat Usher syndrome (e.g., Usher syndrome, type 2A (or type IIA)) or retinitis pigmentosa. The disclosed methods may be used to treat any type of Usher syndrome, such as Usher syndrome type 1, type 2 and/or type 3. In various embodiments, the base editing is used to modify the nucleotide sequence of the USH2A gene such that USH2A protein function is restored, completely or partially. For example, in some embodiments base editing is used to restore US2HA function by disrupting a splice site (or pseudo-splice site) in the USH2A gene sequence to induce skipping of an exon containing a mutation. This approach is referred to herein as the “exon-skipping approach.” In other embodiments, base editing is used to restore US2HA function by correcting a point mutation (e.g., in an exon) so as to eliminate or reduce missense mutations, or eliminate or reduce nonsense mutations. This approach is referred to herein as the “mutation correction approach.” The presently disclosed compositions, kits, and methods make use of recently generated high-efficiency base editors such as BE4max, BE4max-VRQR, ABE7.10, and ABE8e, to install targeted edits in the USH2A gene with low frequency of off-target effects. The presently disclosed compositions, kits, and methods also make use of novel guide RNAs to program the base editor to the correct site in the USH2A gene. Several of thes guide RNAs are particularly useful for the exon-skipping approach, and several are particularly useful for the mutation-correction approach.

Mutations in USH2A are the most prevalent cause of Usher Syndrome (which may cause retinitis pigmentosa, visual loss, and/or hearing loss), with >1000 pathogenic mutations in USH2A reported in humans. Over 300 of these mutations are transition mutations, which may be corrected by base editing. Base editing at potentially therapeutically-relevant efficiencies in murine retina in vivo was recently demonstrated. (See Levy et al., Nat Biomed Eng. 2020 Jan; 4(1):97-110, herein incorporated by reference.) Therapeutic applications of base editing are constrained by the requirement for a protospacer-adjacent motif (PAM) recognition sequence, the potential generation of nearby bystander edits, and potential off-target effects. In cases where bioinformatic design criteria are met, empirical testing is then needed to identify gRNA sequence-base editor combinations with high on-target editing activity.

In the Examples of the present disclosure, a tandem array of 35 USH2A mutant sequences was integrated into the AAVS1 locus in HEK293 cells, allowing for rapid testing of base editor activity at these USH2A disease-associated mutations using a single cell line. These mutant sequences were selected based on bioinformatic design criteria and the prevalence of the mutations in humans. Using adenine base editors (ABEs) and cytidine base editors (CBEs), multiple mutations of USH2A were identified that showed high efficiency of base editing (>50%) with a low indel frequency (˜3%). Off-target effects at targeted editing sites were evaluated empirically for promising targets. There were specific (but limited) differences detected between various testing methods, including i) integrated editing target versus target plasmid transfection; ii) single-plex versus multiplex guide transfection; and iii) Sanger sequencing versus Illumina-based next-generation sequencing (NGS) editing efficacy evaluation. Testing of split-intein editors of the disclosed base editors that can be delivered by AAV in vivo is ongoing. The methods disclosed herein improve the rate and throughput with which promising base editor targets can be identified in cultured cells and in vivo.

In some embodiments, the disclosure provides guide sequences capable of directing base editors (e.g., adenosine base editors or cytidine base editors) to positions within the USH2A gene to correct mutations to treat Usher syndrome. In some embodiments, the disclosure provides guide sequences capable of directing base editors to splice sites of the USH2A gene to disrupt these splice sites to treat Usher syndrome. In some aspects, the disclosure provides proteins that deaminate the nucleobase adenine in the USH2A gene to treat Usher syndrome. In some aspects, the disclosure provides proteins that deaminate the nucleobase cytidine in an USH2A gene to treat Usher syndrome.

Also exemplified herein are cytosine base editors, which are capable of installing C-to-G edits. In some aspects, the disclosure provides proteins that deaminate the nucleobase cytosine in the USH2A gene to treat Usher syndrome.

Any base editors known in the art or to be developed in the future may be used in accordance with the present disclosure. Exemplified herein are adenosine base editors (i.e., capable of installing A-to-G edits, a type of transition edit) to account for a variety of genetic strategies that reduce or eliminate the expression of truncated Usherin (or USH2A) protein.

In some embodiments, the base editor nicks the target sequence that is complementary to the guide sequence. In some embodiments, the base editor may be formulated as a split-intein base editor.

This disclosure describes fusion proteins may be used for targeted editing of DNA in vitro, e.g., for the generation of mutant cells or animals; for the introduction of targeted mutations, e.g., for the correction of genetic defects in cells ex vivo, e.g., in cells obtained from a subject that are subsequently re-introduced into the same or another subject; and for the introduction of targeted mutations in vivo, e.g., the correction of genetic defects or the introduction of deactivating mutations in disease-associated genes in a subject, such as a mammalian subject. As an example, diseases and conditions can be treated by making an A to G, or a T to C mutation, may be treated using the base editors described herein. The base editors described herein may be utilized for the targeted editing of such G to A mutations so as to correct a mutation or restore a normal reading frame in an USH2A gene to generate a functional Usherin protein. The invention provides deaminase proteins, fusion proteins (base editors), nucleic acids, vectors; and cells, compositions, methods, kits, systems, etc. that utilize the disclosed deaminases, base editors, and vectors.

To bypass the packaging size limit and deliver base editors using AAVs, a split-base editor dual AAV strategy was devised, in which the CBE or ABE is divided into an N-terminal and C-terminal half. Each base editor half is fused to half of a fast-splicing split-intein. Following co-infection by AAV particles expressing each base editor—split intein half, protein splicing in trans reconstitutes full-length base editor. Unlike other approaches utilizing small molecules or sgRNA to bridge split Cas9, intein splicing removes all exogenous sequences and regenerates a native peptide bond at the split site, resulting in a single reconstituted protein identical in sequence to the unmodified base editor.

Split-intein CBEs and split-intein ABEs were developed and integrated into optimized dual AAV genomes to enable efficient base editing in somatic tissues of therapeutic relevance, including liver, heart, muscle, retina, and brain. The resulting AAVs were used to achieve base editing efficiencies at test loci for both CBEs and ABEs that, in each of these tissues, meets or exceeds therapeutically relevant editing thresholds for the treatment of some human genetic diseases at AAV dosages that are known to be well-tolerated in humans.

### USH2A and Usher Syndrome

Mutations in the USH2A gene are the most common cause of both non-syndromic retinal degeneration and Usher syndrome, affecting 1 in 6000 people. Patients with both conditions are affected with retinitis pigmentosa, characterized by onset of nyctalopia in teenage years, followed by progressive constriction of the visual field and eventually degradation of central vision. Patients with Usher syndrome also experience congenital, bilateral sensorineural hearing loss that occurs predominantly in the higher frequencies and ranges from severe to profound, resulting in a reduced ability of the individual to perceive, communicate, and extract vital information from their environment.

The protein encoded by the USH2A gene, Usherin, is a large transmembrane protein anchored in the plasma membrane of photoreceptors in the retina and in the hair cells of the cochlea. Its extracellular portion contains many repeated domains, including Laminin EGF-like (LE) domains and 35 Fibronectin type 3 (FN3) domains (FIG. 1A). Together, these repetitive domains comprise over 78% of the protein structure. Usherin is part of Usher protein complex II that is present in photoreceptor cells of the retina and hair cells of the inner ear. In the mouse retina, usherin is localized in the calycal processes of photoreceptor cells, which participates in regulating intracellular protein transport, and lasts for a life time. In mouse cochlear hair cells, usherin expression starts at embryonic stage E18 in the stereocilia bundle of hair cells, which is a highly specialized structure critical for transducing mechanical sound stimuli into electrical signals, and persists during early postnatal stages until approximately postnatal day 15. In addition, Usherin is detected in the synaptic regions of both cell types. Along with other USH2 complex proteins, Ush2a putatively participates in the differentiation of the stereocilia and the renewal of nascent outer segments. Ush2a knockout (KO) mice exhibit slowly progressive photoreceptor degeneration and defective stereocilia formation of hair cells of cochlear basal turn in neonates. Taken together, these indicate that usherin plays inherent role in maintaining the key functional and/or structural features shared between these two types of ciliated sensory neurons.

The human USH2A gene consists of 71 coding exons, 25 of which are in-frame with the remaining transcript (FIG. 1B). To date, over 1000 pathogenic USH2A mutations distributed throughout the genome have been identified, as reported in the Leiden Open Variation Database (LOVD) (http://www.lovd.nl). In particular, mutations in exon 13 account for approximately 35% of all USH2A cases, including a single base deletion at position 2299 (c.2299delG), the most common mutation in USH2A (FIG. 1C). This mutation creates a frameshift in the coding sequence, resulting in a premature stop codon in exon 13. This premature stop is predicted to lead to a null or a truncated, non-functional Usherin protein.

USH2A is one of the largest genes in the human genome, with about 800 kb of genomic sequence and 15.6 kb of coding sequence. This seriously hampers the possibility of traditional gene augmentation therapy, as it far exceeds the packaging capacity of standard gene therapy delivery vectors. In the past decade, alternative approaches for treating the mutations in large genes, such as anti-sense oligonucleotide-mediated splice correction, or the use of mini-gene, have been actively pursued with limited success. Recently, a novel strategy known as exon-skipping has shown great promise and has been successfully applied in pre-clinical and clinical studies to treat genetic diseases, such as Duchene Muscular Dystrophy (DMD), Huntington's diseases and CEP290-associated LCA. Exon-skipping approach is built upon the concept/prediction that when an mutant exon that is in-frame, or a pseudo-exon resulting from an aberrant splicing site in the introns, is excluded from the mRNA (“skipped”), the open reading frame of the remaining transcript is maintained or restored from frame-shifted mutant mRNA. Exon skipping can be achieved transiently at the mRNA level by antisense oligonucleotide (ASOs), RNAi, or CRISPR/Cas RNA editing. Exon skipping can also be attained permanently by directly altering the genomic DNA, for instance using programmable CRISPR genome editing to delete the target exons or disrupt the specific splicing sites of the target exons.

The usherin protein encoded by USH2A (GenBank Ace No. NC_000001.11, Reference GRCh38.p7 Primary Assembly, Range 215622894-216423396, complement; SEQ ID NO: 1) is a transmembrane protein anchored in the photoreceptor plasma membrane (van Wijk, E., et al., Am J Hum Genet, 2004. 74(4): 738-44; Grati, M., et al., J Neurosci, 2012. 32(41): p. 14288-93). Its extracellular portion, which accounts for over 96% of the length of the protein and projects into the periciliary matrix, is thought to have an important structural and a possible signaling role for the long-term maintenance of photoreceptors (van Wijk, 2012; Grati, M., 2012. 32(41): 14288-93). Two isoforms of USH2A have been described. Isoform b (GenBank Acc. No. NM_206933.2 (transcript) and NP 996816.2 (protein)) is most abundantly expressed in retina and is used as the canonical, standard sequence in the literature and in this disclosure. Usherin is a protein with a high degree of homologous domain structures (Liu, X., et al., Proc Natl Acad Sci USA, 2007. 104(11): 4413-18). Intracellularly, a PDZ domain has been identified to bind whirlin, whereas extracellularly, several domains are present and in most cases in a repetitive fashion, including Laminin EGF-like (LE) domains and Fibronectin type 3 (FN3) domains. These repetitive domains comprise over 78% of the protein structure combined. The most common mutation c.2299delG, p.G1u767fs in USH2A gene, which causes approximately 15-30% of USH2A-associated diseases is the United States, is located in exon 13 that encodes LE domain 5 (amino acids 747-794).

Given the high degree of repetitive regions in usherin, it has been hypothesized that an usherin protein that lacks one or more of the repetitive domains would retain partial or complete structural integrity and function, such that the abbreviated USH2A can serve as a therapeutic strategy for Usher syndrome type II and autosomal recessive retinitis pigmentosa (arRP) by skipping the mutant exon in USH2A gene. As described elsewhere, a mutant USH2A gene lacking exon 12, and with exons 11 and 13 fused in-frame, is expressed and localizes correctly in the mouse retina and cochlea. See International Publication No. WO 2019/0183641, published Jun. 20, 2019, herein incorporated by reference. CRISPR/Cas9-based exon-skipping gene editing strategies to restore the reading frame of USH2A by deleting exon 13 holds therapeutic potential for the treatment of USH2A patients.

Through a series of in vitro and in vivo experiments, it was shown that a mutant Ush2A protein that lacks repetitive LE domains encoded by mouse exon 12 (referred to as USH2A-ΔEx13 or Ush2a-ΔEx12) retains its biological function. See Pendse et al., “Exon 13-skipped USH2A protein retains functional integrity in mice, suggesting an exon-skipping therapeutic approach to treat USH2A-associated disease.” bioRxiv 2020.02.04.934240 (2020), herein incorporated by reference. Mouse exon 12 is homologous to human USH2A exon 13. The mutant Usherin protein was generated by CRISPR/Cas9-mediated clavage and knockout. This shortened version of USH2A-ΔEx13 was shown to be capable of rescuing the impaired ciliogenesis in an Ush2a null cell line. A single copy of Ush2a-ΔEx12 completely restored hair cell morphology and fully restored auditory function in the inner ear and mitigated early abnormalities in mouse retina. The proof-of-concept study of Pendse et al. demonstrated the substantial therapeutic potential of exon skipping therapies for the treatment of USH2A-associated disease caused by mutations in exon 13, which represents over one third of all USH2A cases. The exon-skipping approach of the base editing methods disclosed herein is based, at least in part, on this discovery.

Recently, Sanjurjo-Soriano and others demonstrated the feasibility of using CRISPR/eSpCas9-mediated genome editing to correct the two most prevalent USH2A mutations, c.2276G>T and c.2299delG, in induced pluripotent stem cells (iPSCs) of human patients. See Sanjurjo-Soriano et al., Molecular Therapy: Methods & Clinical Development Vol. 17 (Jun. 2020), incorporated herein by reference. This group generated the eSpCas9 variant by the introduction of several alanine substitutions in the wild-type protein sequence, which weaken the ionic interactions between the protein and the target DNA. These substitutions trap the eSp-Cas9 in an inactive state when it is bound to mismatched targets.

napDNAbp Domains

In one aspect, the editing methods and base editors described herein involve a nucleic acid programmable DNA binding protein (napDNAbp). Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence. In various embodiments, the napDNAbp can be fused to a herein disclosed adenosine deaminase.

Without being bound by theory, the binding mechanism of a napDNAbp — guide RNA complex, in general, includes the step of forming an R-loop whereby the napDNAbp induces the unwinding of a double-strand DNA target, thereby separating the strands in the region bound by the napDNAbp. The guide RNA spacer then hybridizes to the “target strand.” This displaces a “non-target strand” that is complementary to the target strand, which forms the single strand region of the R-loop. In some embodiments, the napDNAbp includes one or more nuclease activities, which then cut the DNA leaving various types of lesions. For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”).

The below description of various napDNAbps which can be used in connection with the presently disclose base editors is not meant to be limiting in any way. The base editors may comprise the canonical SpCas9, or any ortholog Cas9 protein, or any variant Cas9 protein—including any naturally occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave of strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats). The base editors described herein may also comprise Cas9 equivalents, including Cas12a/Cpf1 and Cas12b proteins which are the result of convergent evolution. The napDNAbps used herein (e.g., SpCas9, Cas9 variant, or Cas9 equivalents) may also may also contain various modifications that alter/enhance their PAM specificities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a reference SpCas9 canonical sequence or a reference Cas9 equivalent (e.g., Cas12a/Cpf1).

The napDNAbp can be a CRISPR (clustered regularly interspaced short palindromic repeat)-associated nuclease. As outlined above, CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M. et al., Science 337:816-821(2012), the entire contents of which is hereby incorporated by reference.

In some embodiments, the napDNAbp directs cleavage of one or both strands at the location of a target sequence, such as within the target sequence and/or within the complement of the target sequence. In some embodiments, the napDNAbp directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a napDNAbp that is mutated to with respect to a corresponding wild-type enzyme such that the mutated napDNAbp lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A in reference to the canonical SpCas9 sequence, or to equivalent amino acid positions in other Cas9 variants or Cas9 equivalents.

As used herein, the term “Cas protein” refers to a full-length Cas protein obtained from nature, a recombinant Cas protein having a sequences that differs from a naturally occurring Cas protein, or any fragment of a Cas protein that nevertheless retains all or a significant amount of the requisite basic functions needed for the disclosed methods, i.e., (i) possession of nucleic-acid programmable binding of the Cas protein to a target DNA, and (ii) ability to nick the target DNA sequence on one strand. The Cas proteins contemplated herein embrace CRISPR Cas 9 proteins, as well as Cas9 equivalents, variants (e.g., Cas9 nickase (nCas9) or nuclease inactive Cas9 (dCas9)) homologs, orthologs, or paralogs, whether naturally occurring or non-naturally occurring (e.g., engineered or recombinant), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference.

The terms “Cas9” or “Cas9 nuclease” or “Cas9 moiety” or “Cas9 domain” embrace any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular Cas9 that is employed in the base editor (BE) of the invention.

As noted herein, Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J.A., Charpentier E. Science 337:816-821(2012), the entire contents of each of which are incorporated herein by reference).

Examples of Cas9 and Cas9 equivalents are provided as follows; however, these specific examples are not meant to be limiting. The base editor fusions of the present disclosure may use any suitable napDNAbp, including any suitable Cas9 or Cas9 equivalent.

Exemplary napDNAbp domains include, but are not limited to S. pyogenes Cas9 nickase (SpCas9n) and S. aureus Cas9 nickase (SaCas9n). Wild-type SpCas9 and SaCas9 are also contemplated as exemplary napDNAbp domains. Additional exemplary napDNAbp domains include S. aureus Cas9-KKH (SaCas9-KKH), LbCas12a, enAsCas12a (an engineered AsCas12a recently reported by Joung et al.), SpCas9-NG, SpCas9-NG-CP1041, SpCas9-NG-VRQR, SpCas9-NG-VRQR, SpCas9-VRQR, SpCas9-NRCH, CP1028-SpCas9, and CP1041-SpCas9. In some embodiments, the napDNAbp domain comprises a Cas9 nickase (nCas9) domain. In some embodiments, the napDNAbp domain comprises an SpCas9n domain. In some embodiments, the napDNAbp domain is an SpCas9-VQR domain. In some embodiments, the napDNAbp domain is an SpCas9-VRQR or an SpCas9-VRER domain. In certain embodiments, the napDNAbp domain of any of the disclosed base editors is a SaCas9n.

In certain embodiments, the napDNAbp domain of any of the disclosed base editors is an SpCas9-NRCH. In certain embodiments, the napDNAbp domain of any of the disclosed base editors is an LbCas12a, e.g., a catalytically inactive or “dead” LbCas12a. In certain embodiments, the napDNAbp domain of any of the disclosed base editors is an AsCas12a, e.g., an enAsCas12a. In certain embodiments, the napDNAbp domain of any of the disclosed base editors is a cicular permuted variant of SpCas9, e.g., a CP1028-SpCas9 or a CP1041-SpCas9. In certain embodiments, the napDNAbp domain of any of the disclosed base editors is an evolved SpCas9, e.g., an SpCas9-NG. In certain embodiments, the napDNAbp domain of any of the disclosed base editors has an amino acid sequence that is at least 85%, 90%, 92.5%, 95%, 98%, or 99% identical to any one of SEQ ID NOs: 101, 102, 107, 118, 120, 133-134, 153-157, 171-175, 181, 182, and 436-437. In certain embodiments, the napDNAbp domain of any of the disclosed base editors comprises the amino acid sequence of any one of SEQ ID NOs: 101, 102, 107, 118, 120, 133-134, 153-157, 171-175, 181, 182, and 436-437.

In certain embodiments, the napDNAbp domain of any of the disclosed base editors comprises any one of Steptococcus cams Cas9 (ScCas9), St1Cas9, SpEQRCas9, and SpVRERCas9 (see WO 2019/217942, published Nov. 14, 2019, and WO 2019/217943, published Nov. 14, 2019, each of which is incorporated herein by reference). In certain embodiments, the napDNAbp domain of any of the disclosed base editors comprises a heterodimer of two Cas domains, such as dead Cas9 and nCas9 (see, e.g., WO 2020/051562, published Mar. 12, 2020, which is incorporated herein by reference).

### (i) Wild Type Canonical SpCas9

In one embodiment, the base editor constructs described herein may comprise the “canonical SpCas9” nuclease from S. pyogenes, which has been widely used as a tool for genome engineering. This Cas9 protein is a large, multi-domain protein containing two distinct nuclease domains. Point mutations can be introduced into Cas9 to abolish one or both nuclease activities, resulting in a nickase Cas9 (nCas9) or dead Cas9 (dCas9), respectively, that still retains its ability to bind DNA in a sgRNA-programmed manner. In principle, when fused to another protein or domain, Cas9 or variant thereof (e.g., nCas9) can target that protein to virtually any DNA sequence simply by co-expression with an appropriate sgRNA. As used herein, the canonical SpCas9 protein refers to the wild type protein from Streptococcus pyogenes having the following amino acid sequence:

| Description | Sequence | SEQ ID NO: |
|---|---|---|
| SpCas 9 | MDKKYSIGLDIGTNSVGWAVITDEYKVPSKKFKVLGNTDRHSIKKNLIGALLFDS | SEQ ID NO: 74 |
| Streptococcus | GETAEATRLKRTARRRYTRRKNRICYLQEIFSNEMAKVDDSFFHRLEESFLVEED | |
| pyogenes | KKHERHPIFGNIVDEVAYHEKYPTIYHLRKKLVDSTDKADLRLIYLALAHMIKFR | |
| M1 | GHFLIEGDLNPDNSDVDKLFIQLVQTYNQLFEENPINASGVDAKAILSARLSKSR | |
| SwissProt | RLENLIAQLPGEKKNGLFGNLIALSLGLTPNFKSNFDLAEDAKLQLSKDTYDDDL | |
| Accession | DNLLAQIGDQYADLFLAAKNLSDAILLSDILRVNTEITKAPLSASMIKRYDEHHQ | |
| No. Q99ZW2 | DLTLLKALVRQQLPEKYKEIFFDQSKNGYAGYIDGGASQEEFYKFIKPILEKMDG | |
| Wild type | TEELLVKLNREDLLRKQRTFDNGSIPHQIHLGELHAILRRQEDFYPFLKDNREKI | |
| | EKILTFRIPYYVGPLARGNSRFAWMTRKSEETITPWNFEEVVDKGASAQSFIERM | |
| | TNFDKNLPNEKVLPKHSLLYEYFTVYNELTKVKYVTEGMRKPAFLSGEQKKAIVD | |
| | LLFKTNRKVTVKQLKEDYFKKIECFDSVEISGVEDRFNASLGTYHDLLKIIKDKD | |
| | FLDNEENEDILEDIVLTLTLFEDREMIEERLKTYAHLFDDKVMKQLKRRRYTGWG | |
| | RLSRKLINGIRDKQSGKTILDFLKSDGFANRNFMQLIHDDSLTFKEDIQKAQVSG | |
| | QGDSLHEHIANLAGSPAIKKGILQTVKVVDELVKVMGRHKPENIVIEMARENQTT | |
| | QKGQKNSRERMKRIEEGIKELGSQILKEHPVENTQLQNEKLYLYYLQNGRDMYVD | |
| | QELDINRLSDYDVDHIVPQSFLKDDSIDNKVLTRSDKNRGKSDNVPSEEVVKKMK | |
| | NYWRQLLNAKLITQRKFDNLTKAERGGLSELDKAGFIKRQLVETRQITKHVAQIL | |
| | DSRMNTKYDENDKLIREVKVITLKSKLVSDFRKDFQFYKVREINNYHHAHDAYLN | |
| | AVVGTALIKKYPKLESEFVYGDYKVYDVRKMIAKSEQEIGKATAKYFFYSNIMNF | |
| | FKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSK | |
| | KLKSVKELLGITIMERSSFEKNPIDFLEAKGYKEVKKDLIIKLPKYSLFELENGR | |
| | KRMLASAGELQKGNELALPSKYVNFLYLASHYEKLKGSPEDNEQKQLFVEQHKHY | |
| | LDEIIEQISEFSKRVILADANLDKVLSAYNKHRDKPIREQAENIIHLFTLTNLGA | |
| | PAAFKYFDTTIDRKRYTSTKEVLDATLIHQSITGLYETRIDLSQLGGD | |
| SpCas 9 | ATGGATAAAAAATATAGCATTGGCCTGGATATTGGCACCAACAGCGTGGGCTGGG | SEQ ID NO: 75 |
| Reverse | CGGTGATTACCGATGAATATAAAGTGCCGAGCAAAAAATTTAAAGTGCTGGGCAA | |
| translation | CACCGATCGCCATAGCATTAAAAAAAACCTGATTGGCGCGCTGCTGTTTGATAGC | |
| of | GGCGAAACCGCGGAAGCGACCCGCCTGAAACGCACCGCGCGCCGCCGCTATACCC | |
| SwissProt | GCCGCAAAAACCGCATTTGCTATCTGCAGGAAATTTTTAGCAACGAAATGGCGAA | |
| Accession | AGTGGATGATAGCTTTTTTCATCGCCTGGAAGAAAGCTTTCTGGTGGAAGAAGAT | |
| No. Q99ZW2 | AAAAAACATGAACGCCATCCGATTTTTGGCAACATTGTGGATGAAGTGGCGTATC | |
| Streptococcus | ATGAAAAATATCCGACCATTTATCATCTGCGCAAAAAACTGGTGGATAGCACCGA | |
| pyogenes | TAAAGCGGATCTGCGCCTGATTTATCTGGCGCTGGCGCATATGATTAAATTTCGC | |
| | GGCCATTTTCTGATTGAAGGCGATCTGAACCCGGATAACAGCGATGTGGATAAAC | |
| | TGTTTATTCAGCTGGTGCAGACCTATAACCAGCTGTTTGAAGAAAACCCGATTAA | |
| | CGCGAGCGGCGTGGATGCGAAAGCGATTCTGAGCGCGCGCCTGAGCAAAAGCCGC | |
| | CGCCTGGAAAACCTGATTGCGCAGCTGCCGGGCGAAAAAAAAAACGGCCTGTTTG | |
| | GCAACCTGATTGCGCTGAGCCTGGGCCTGACCCCGAACTTTAAAAGCAACTTTGA | |
| | TCTGGCGGAAGATGCGAAACTGCAGCTGAGCAAAGATACCTATGATGATGATCTG | |
| | GATAACCTGCTGGCGCAGATTGGCGATCAGTATGCGGATCTGTTTCTGGCGGCGA | |
| | AAAACCTGAGCGATGCGATTCTGCTGAGCGATATTCTGCGCGTGAACACCGAAAT | |
| | TACCAAAGCGCCGCTGAGCGCGAGCATGATTAAACGCTATGATGAACATCATCAG | |
| | GATCTGACCCTGCTGAAAGCGCTGGTGCGCCAGCAGCTGCCGGAAAAATATAAAG | |
| | AAATTTTTTTTGATCAGAGCAAAAACGGCTATGCGGGCTATATTGATGGCGGCGC | |
| | GAGCCAGGAAGAATTTTATAAATTTATTAAACCGATTCTGGAAAAAATGGATGGC | |
| | ACCGAAGAACTGCTGGTGAAACTGAACCGCGAAGATCTGCTGCGCAAACAGCGCA | |
| | CCTTTGATAACGGCAGCATTCCGCATCAGATTCATCTGGGCGAACTGCATGCGAT | |
| | TCTGCGCCGCCAGGAAGATTTTTATCCGTTTCTGAAAGATAACCGCGAAAAAATT | |
| | GAAAAAATTCTGACCTTTCGCATTCCGTATTATGTGGGCCCGCTGGCGCGCGGCA | |
| | ACAGCCGCTTTGCGTGGATGACCCGCAAAAGCGAAGAAACCATTACCCCGTGGAA | |
| | CTTTGAAGAAGTGGTGGATAAAGGCGCGAGCGCGCAGAGCTTTATTGAACGCATG | |
| | ACCAACTTTGATAAAAACCTGCCGAACGAAAAAGTGCTGCCGAAACATAGCCTGC | |
| | TGTATGAATATTTTACCGTGTATAACGAACTGACCAAAGTGAAATATGTGACCGA | |
| | AGGCATGCGCAAACCGGCGTTTCTGAGCGGCGAACAGAAAAAAGCGATTGTGGAT | |
| | CTGCTGTTTAAAACCAACCGCAAAGTGACCGTGAAACAGCTGAAAGAAGATTATT | |
| | TTAAAAAAATTGAATGCTTTGATAGCGTGGAAATTAGCGGCGTGGAAGATCGCTT | |
| | TAACGCGAGCCTGGGCACCTATCATGATCTGCTGAAAATTATTAAAGATAAAGAT | |
| | TTTCTGGATAACGAAGAAAACGAAGATATTCTGGAAGATATTGTGCTGACCCTGA | |
| | CCCTGTTTGAAGATCGCGAAATGATTGAAGAACGCCTGAAAACCTATGCGCATCT | |
| | GTTTGATGATAAAGTGATGAAACAGCTGAAACGCCGCCGCTATACCGGCTGGGGC | |
| | CGCCTGAGCCGCAAACTGATTAACGGCATTCGCGATAAACAGAGCGGCAAAACCA | |
| | TTCTGGATTTTCTGAAAAGCGATGGCTTTGCGAACCGCAACTTTATGCAGCTGAT | |
| | TCATGATGATAGCCTGACCTTTAAAGAAGATATTCAGAAAGCGCAGGTGAGCGGC | |
| | CAGGGCGATAGCCTGCATGAACATATTGCGAACCTGGCGGGCAGCCCGGCGATTA | |
| | AAAAAGGCATTCTGCAGACCGTGAAAGTGGTGGATGAACTGGTGAAAGTGATGGG | |
| | CCGCCATAAACCGGAAAACATTGTGATTGAAATGGCGCGCGAAAACCAGACCACC | |
| | CAGAAAGGCCAGAAAAACAGCCGCGAACGCATGAAACGCATTGAAGAAGGCATTA | |
| | AAGAACTGGGCAGCCAGATTCTGAAAGAACATCCGGTGGAAAACACCCAGCTGCA | |
| | GAACGAAAAACTGTATCTGTATTATCTGCAGAACGGCCGCGATATGTATGTGGAT | |
| | CAGGAACTGGATATTAACCGCCTGAGCGATTATGATGTGGATCATATTGTGCCGC | |
| | AGAGCTTTCTGAAAGATGATAGCATTGATAACAAAGTGCTGACCCGCAGCGATAA | |
| | AAACCGCGGCAAAAGCGATAACGTGCCGAGCGAAGAAGTGGTGAAAAAAATGAAA | |
| | AACTATTGGCGCCAGCTGCTGAACGCGAAACTGATTACCCAGCGCAAATTTGATA | |
| | ACCTGACCAAAGCGGAACGCGGCGGCCTGAGCGAACTGGATAAAGCGGGCTTTAT | |
| | TAAACGCCAGCTGGTGGAAACCCGCCAGATTACCAAACATGTGGCGCAGATTCTG | |
| | GATAGCCGCATGAACACCAAATATGATGAAAACGATAAACTGATTCGCGAAGTGA | |
| | AAGTGATTACCCTGAAAAGCAAACTGGTGAGCGATTTTCGCAAAGATTTTCAGTT | |
| | TTATAAAGTGCGCGAAATTAACAACTATCATCATGCGCATGATGCGTATCTGAAC | |
| | GCGGTGGTGGGCACCGCGCTGATTAAAAAATATCCGAAACTGGAAAGCGAATTTG | |
| | TGTATGGCGATTATAAAGTGTATGATGTGCGCAAAATGATTGCGAAAAGCGAACA | |
| | GGAAATTGGCAAAGCGACCGCGAAATATTTTTTTTATAGCAACATTATGAACTTT | |
| | TTTAAAACCGAAATTACCCTGGCGAACGGCGAAATTCGCAAACGCCCGCTGATTG | |
| | AAACCAACGGCGAAACCGGCGAAATTGTGTGGGATAAAGGCCGCGATTTTGCGAC | |
| | CGTGCGCAAAGTGCTGAGCATGCCGCAGGTGAACATTGTGAAAAAAACCGAAGTG | |
| | CAGACCGGCGGCTTTAGCAAAGAAAGCATTCTGCCGAAACGCAACAGCGATAAAC | |
| | TGATTGCGCGCAAAAAAGATTGGGATCCGAAAAAATATGGCGGCTTTGATAGCCC | |
| | GACCGTGGCGTATAGCGTGCTGGTGGTGGCGAAAGTGGAAAAAGGCAAAAGCAAA | |
| | AAACTGAAAAGCGTGAAAGAACTGCTGGGCATTACCATTATGGAACGCAGCAGCT | |
| | TTGAAAAAAACCCGATTGATTTTCTGGAAGCGAAAGGCTATAAAGAAGTGAAAAA | |
| | AGATCTGATTATTAAACTGCCGAAATATAGCCTGTTTGAACTGGAAAACGGCCGC | |
| | AAACGCATGCTGGCGAGCGCGGGCGAACTGCAGAAAGGCAACGAACTGGCGCTGC | |
| | CGAGCAAATATGTGAACTTTCTGTATCTGGCGAGCCATTATGAAAAACTGAAAGG | |
| | CAGCCCGGAAGATAACGAACAGAAACAGCTGTTTGTGGAACAGCATAAACATTAT | |
| | CTGGATGAAATTATTGAACAGATTAGCGAATTTAGCAAACGCGTGATTCTGGCGG | |
| | ATGCGAACCTGGATAAAGTGCTGAGCGCGTATAACAAACATCGCGATAAACCGAT | |
| | TCGCGAACAGGCGGAAAACATTATTCATCTGTTTACCCTGACCAACCTGGGCGCG | |
| | CCGGCGGCGTTTAAATATTTTGATACCACCATTGATCGCAAACGCTATACCAGCA | |
| | CCAAAGAAGTGCTGGATGCGACCCTGATTCATCAGAGCATTACCGGCCTGTATGA | |
| | AACCCGCATTGATCTGAGCCAGCTGGGCGGCGAT | |

The base editors described herein may include canonical SpCas9, or any variant thereof having at least 80%, at least 85%, at least 90%, at least 95%, or at least 99% sequence identity with a wild type Cas9 sequence provided above. These variants may include SpCas9 variants containing one or more mutations, including any known mutation reported with the SwissProt Accession No. Q99ZW2 entry, which include:

| SpCas9 mutation (relative | Function/Characteristic (as reported) |
|---|---|
| to the amino acid sequence | (see UniProtKB—Q99ZW2 |
| of the canonical SpCas9 | (CAS9_STRPT1) entry— |
| sequence, SEQ ID NO: 74) | incorporated herein by reference) |
| D10A | Nickase mutant which cleaves the |
| | protospacer strand (but no cleavage |
| | of non-protospacer strand) |
| S15A | Decreased DNA cleavage activity |
| R66A | Decreased DNA cleavage activity |
| R70A | No DNA cleavage |
| R74A | Decreased DNA cleavage |
| R78A | Decreased DNA cleavage |
| 97-150 deletion | No nuclease activity |
| R165A | Decreased DNA cleavage |
| 175-307 deletion | About 50% decreased DNA cleavage |
| 312-409 deletion | No nuclease activity |
| E762A | Nickase |
| H840A | Nickase mutant which cleaves the |
| | non-protospacer strand but does not |
| | cleave the protospacer strand |
| N854A | Nickase |
| N863A | Nickase |
| H982A | Decreased DNA cleavage |
| D986A | Nickase |
| 1099-1368 deletion | No nuclease activity |
| R1333A | Reduced DNA binding |

Other wild type SpCas9 sequences that may be used in the present disclosure, include:

| Description | Sequence | SEQ ID NO: |
|---|---|---|
| SpCas9 | ATGGATAAGAAATACTCAATAGGCTTAGATATCGGCACAAATAGCGTCGGATGGGCG | SEQ ID NO: 76 |
| Streptococcus | GTGATCACTGATGATTATAAGGTTCCGTCTAAAAAGTTCAAGGTTCTGGGAAATACA | |
| pyogenes | GACCGCCACAGTATCAAAAAAAATCTTATAGGGGCTCTTTTATTTGGCAGTGGAGAG | |
| MGAS1882 | ACAGCGGAAGCGACTCGTCTCAAACGGACAGCTCGTAGAAGGTATACACGTCGGAAG | |
| wild type | AATCGTATTTGTTATCTACAGGAGATTTTTTCAAATGAGATGGCGAAAGTAGATGAT | |
| NC_017053.1 | AGTTTCTTTCATCGACTTGAAGAGTCTTTTTTGGTGGAAGAAGACAAGAAGCATGAA | |
| | CGTCATCCTATTTTTGGAAATATAGTAGATGAAGTTGCTTATCATGAGAAATATCCA | |
| | ACTATCTATCATCTGCGAAAAAAATTGGCAGATTCTACTGATAAAGCGGATTTGCGC | |
| | TTAATCTATTTGGCCTTAGCGCATATGATTAAGTTTCGTGGTCATTTTTTGATTGAG | |
| | GGAGATTTAAATCCTGATAATAGTGATGTGGACAAACTATTTATCCAGTTGGTACAA | |
| | ATCTACAATCAATTATTTGAAGAAAACCCTATTAACGCAAGTAGAGTAGATGCTAAA | |
| | GCGATTCTTTCTGCACGATTGAGTAAATCAAGACGATTAGAAAATCTCATTGCTCAG | |
| | CTCCCCGGTGAGAAGAGAAATGGCTTGTTTGGGAATCTCATTGCTTTGTCATTGGGA | |
| | TTGACCCCTAATTTTAAATCAAATTTTGATTTGGCAGAAGATGCTAAATTACAGCTT | |
| | TCAAAAGATACTTACGATGATGATTTAGATAATTTATTGGCGCAAATTGGAGATCAA | |
| | TATGCTGATTTGTTTTTGGCAGCTAAGAATTTATCAGATGCTATTTTACTTTCAGAT | |
| | ATCCTAAGAGTAAATAGTGAAATAACTAAGGCTCCCCTATCAGCTTCAATGATTAAG | |
| | CGCTACGATGAACATCATCAAGACTTGACTCTTTTAAAAGCTTTAGTTCGACAACAA | |
| | CTTCCAGAAAAGTATAAAGAAATCTTTTTTGATCAATCAAAAAACGGATATGCAGGT | |
| | TATATTGATGGGGGAGCTAGCCAAGAAGAATTTTATAAATTTATCAAACCAATTTTA | |
| | GAAAAAATGGATGGTACTGAGGAATTATTGGTGAAACTAAATCGTGAAGATTTGCTG | |
| | CGCAAGCAACGGACCTTTGACAACGGCTCTATTCCCCATCAAATTCACTTGGGTGAG | |
| | CTGCATGCTATTTTGAGAAGACAAGAAGACTTTTATCCATTTTTAAAAGACAATCGT | |
| | GAGAAGATTGAAAAAATCTTGACTTTTCGAATTCCTTATTATGTTGGTCCATTGGCG | |
| | CGTGGCAATAGTCGTTTTGCATGGATGACTCGGAAGTCTGAAGAAACAATTACCCCA | |
| | TGGAATTTTGAAGAAGTTGTCGATAAAGGTGCTTCAGCTCAATCATTTATTGAACGC | |
| | ATGACAAACTTTGATAAAAATCTTCCAAATGAAAAAGTACTACCAAAACATAGTTTG | |
| | CTTTATGAGTATTTTACGGTTTATAACGAATTGACAAAGGTCAAATATGTTACTGAG | |
| | GGAATGCGAAAACCAGCATTTCTTTCAGGTGAACAGAAGAAAGCCATTGTTGATTTA | |
| | CTCTTCAAAACAAATCGAAAAGTAACCGTTAAGCAATTAAAAGAAGATTATTTCAAA | |
| | AAAATAGAATGTTTTGATAGTGTTGAAATTTCAGGAGTTGAAGATAGATTTAATGCT | |
| | TCATTAGGCGCCTACCATGATTTGCTAAAAATTATTAAAGATAAAGATTTTTTGGAT | |
| | AATGAAGAAAATGAAGATATCTTAGAGGATATTGTTTTAACATTGACCTTATTTGAA | |
| | GATAGGGGGATGATTGAGGAAAGACTTAAAACATATGCTCACCTCTTTGATGATAAG | |
| | GTGATGAAACAGCTTAAACGTCGCCGTTATACTGGTTGGGGACGTTTGTCTCGAAAA | |
| | TTGATTAATGGTATTAGGGATAAGCAATCTGGCAAAACAATATTAGATTTTTTGAAA | |
| | TCAGATGGTTTTGCCAATCGCAATTTTATGCAGCTGATCCATGATGATAGTTTGACA | |
| | TTTAAAGAAGATATTCAAAAAGCACAGGTGTCTGGACAAGGCCATAGTTTACATGAA | |
| | CAGATTGCTAACTTAGCTGGCAGTCCTGCTATTAAAAAAGGTATTTTACAGACTGTA | |
| | AAAATTGTTGATGAACTGGTCAAAGTAATGGGGCATAAGCCAGAAAATATCGTTATT | |
| | GAAATGGCACGTGAAAATCAGACAACTCAAAAGGGCCAGAAAAATTCGCGAGAGCGT | |
| | ATGAAACGAATCGAAGAAGGTATCAAAGAATTAGGAAGTCAGATTCTTAAAGAGCAT | |
| | CCTGTTGAAAATACTCAATTGCAAAATGAAAAGCTCTATCTCTATTATCTACAAAAT | |
| | GGAAGAGACATGTATGTGGACCAAGAATTAGATATTAATCGTTTAAGTGATTATGAT | |
| | GTCGATCACATTGTTCCACAAAGTTTCATTAAAGACGATTCAATAGACAATAAGGTA | |
| | CTAACGCGTTCTGATAAAAATCGTGGTAAATCGGATAACGTTCCAAGTGAAGAAGTA | |
| | GTCAAAAAGATGAAAAACTATTGGAGACAACTTCTAAACGCCAAGTTAATCACTCAA | |
| | CGTAAGTTTGATAATTTAACGAAAGCTGAACGTGGAGGTTTGAGTGAACTTGATAAA | |
| | GCTGGTTTTATCAAACGCCAATTGGTTGAAACTCGCCAAATCACTAAGCATGTGGCA | |
| | CAAATTTTGGATAGTCGCATGAATACTAAATACGATGAAAATGATAAACTTATTCGA | |
| | GAGGTTAAAGTGATTACCTTAAAATCTAAATTAGTTTCTGACTTCCGAAAAGATTTC | |
| | CAATTCTATAAAGTACGTGAGATTAACAATTACCATCATGCCCATGATGCGTATCTA | |
| | AATGCCGTCGTTGGAACTGCTTTGATTAAGAAATATCCAAAACTTGAATCGGAGTTT | |
| | GTCTATGGTGATTATAAAGTTTATGATGTTCGTAAAATGATTGCTAAGTCTGAGCAA | |
| | GAAATAGGCAAAGCAACCGCAAAATATTTCTTTTACTCTAATATCATGAACTTCTTC | |
| | AAAACAGAAATTACACTTGCAAATGGAGAGATTCGCAAACGCCCTCTAATCGAAACT | |
| | AATGGGGAAACTGGAGAAATTGTCTGGGATAAAGGGCGAGATTTTGCCACAGTGCGC | |
| | AAAGTATTGTCCATGCCCCAAGTCAATATTGTCAAGAAAACAGAAGTACAGACAGGC | |
| | GGATTCTCCAAGGAGTCAATTTTACCAAAAAGAAATTCGGACAAGCTTATTGCTCGT | |
| | AAAAAAGACTGGGATCCAAAAAAATATGGTGGTTTTGATAGTCCAACGGTAGCTTAT | |
| | TCAGTCCTAGTGGTTGCTAAGGTGGAAAAAGGGAAATCGAAGAAGTTAAAATCCGTT | |
| | AAAGAGTTACTAGGGATCACAATTATGGAAAGAAGTTCCTTTGAAAAAAATCCGATT | |
| | GACTTTTTAGAAGCTAAAGGATATAAGGAAGTTAAAAAAGACTTAATCATTAAACTA | |
| | CCTAAATATAGTCTTTTTGAGTTAGAAAACGGTCGTAAACGGATGCTGGCTAGTGCC | |
| | GGAGAATTACAAAAAGGAAATGAGCTGGCTCTGCCAAGCAAATATGTGAATTTTTTA | |
| | TATTTAGCTAGTCATTATGAAAAGTTGAAGGGTAGTCCAGAAGATAACGAACAAAAA | |
| | CAATTGTTTGTGGAGCAGCATAAGCATTATTTAGATGAGATTATTGAGCAAATCAGT | |
| | GAATTTTCTAAGCGTGTTATTTTAGCAGATGCCAATTTAGATAAAGTTCTTAGTGCA | |
| | TATAACAAACATAGAGACAAACCAATACGTGAACAAGCAGAAAATATTATTCATTTA | |
| | TTTACGTTGACGAATCTTGGAGCTCCCGCTGCTTTTAAATATTTTGATACAACAATT | |
| | GATCGTAAACGATATACGTCTACAAAAGAAGTTTTAGATGCCACTCTTATCCATCAA | |
| | TCCATCACTGGTCTTTATGAAACACGCATTGATTTGAGTCAGCTAGGAGGTGACTGA | |
| SpCas9 | MDKKYSIGLDIGTNSVGWAVITDDYKVPSKKFKVLGNTDRHSIKKNLIGALLFGSGE | SEQ ID NO: 77 |
| Streptococcus | TAEATRLKRTARRRYTRRKNRICYLQEIFSNEMAKVDDSFFHRLEESFLVEEDKKHE | |
| pyogenes | RHPIFGNIVDEVAYHEKYPTIYHLRKKLADSTDKADLRLIYLALAHMIKFRGHFLIE | |
| MGAS1882 | GDLNPDNSDVDKLFIQLVQIYNQLFEENPINASRVDAKAILSARLSKSRRLENLIAQ | |
| wild type | LPGEKRNGLFGNLIALSLGLTPNFKSNFDLAEDAKLQLSKDTYDDDLDNLLAQIGDQ | |
| NC_017053.1 | YADLFLAAKNLSDAILLSDILRVNSEITKAPLSASMIKRYDEHHQDLTLLKALVRQQ | |
| | LPEKYKEIFFDQSKNGYAGYIDGGASQEEFYKFIKPILEKMDGTEELLVKLNREDLL | |
| | RKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGAYHDLLKIIKDKDFLDNEENEDILEDIVLTLTLFE | |
| | DRGMIEERLKTYAHLFDDKVMKQLKRRRYTGWGRLSRKLINGIRDKQSGKTILDFLK | |
| | SDGFANRNFMQLIHDDSLTFKEDIQKAQVSGQGHSLHEQIANLAGSPAIKKGILQTV | |
| | KIVDELVKVMGHKPENIVIEMARENQTTQKGQKNSRERMKRIEEGIKELGSQILKEH | |
| | PVENTQLQNEKLYLYYLQNGRDMYVDQELDINRLSDYDVDHIVPQSFIKDDSIDNKV | |
| | LTRSDKNRGKSDNVPSEEVVKKMKNYWRQLLNAKLITQRKFDNLTKAERGGLSELDK | |
| | AGFIKRQLVETRQITKHVAQILDSRMNTKYDENDKLIREVKVITLKSKLVSDFRKDF | |
| | QFYKVREINNYHHAHDAYLNAVVGTALIKKYPKLESEFVYGDYKVYDVRKMIAKSEQ | |
| | EIGKATAKYFFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKYGGFDSPTVAY | |
| | SVLVVAKVEKGKSKKLKSVKELLGITIMERSSFEKNPIDFLEAKGYKEVKKDLIIKL | |
| | PKYSLFELENGRKRMLASAGELQKGNELALPSKYVNFLYLASHYEKLKGSPEDNEQK | |
| | QLFVEQHKHYLDEIIEQISEFSKRVILADANLDKVLSAYNKHRDKPIREQAENIIHL | |
| | FTLTNLGAPAAFKYFDTTIDRKRYTSTKEVLDATLIHQSITGLYETRIDLSQLGGD | |
| SpCas9 | ATGGATAAAAAGTATTCTATTGGTTTAGACATCGGCACTAATTCCGTTGGATGGGCT | SEQ ID NO: 78 |
| Streptococcus | GTCATAACCGATGAATACAAAGTACCTTCAAAGAAATTTAAGGTGTTGGGGAACACA | |
| pyogenes | GACCGTCATTCGATTAAAAAGAATCTTATCGGTGCCCTCCTATTCGATAGTGGCGAA | |
| wild type | ACGGCAGAGGCGACTCGCCTGAAACGAACCGCTCGGAGAAGGTATACACGTCGCAAG | |
| SWBC2D7W014 | AACCGAATATGTTACTTACAAGAAATTTTTAGCAATGAGATGGCCAAAGTTGACGAT | |
| | TCTTTCTTTCACCGTTTGGAAGAGTCCTTCCTTGTCGAAGAGGACAAGAAACATGAA | |
| | CGGCACCCCATCTTTGGAAACATAGTAGATGAGGTGGCATATCATGAAAAGTACCCA | |
| | ACGATTTATCACCTCAGAAAAAAGCTAGTTGACTCAACTGATAAAGCGGACCTGAGG | |
| | TTAATCTACTTGGCTCTTGCCCATATGATAAAGTTCCGTGGGCACTTTCTCATTGAG | |
| | GGTGATCTAAATCCGGACAACTCGGATGTCGACAAACTGTTCATCCAGTTAGTACAA | |
| | ACCTATAATCAGTTGTTTGAAGAGAACCCTATAAATGCAAGTGGCGTGGATGCGAAG | |
| | GCTATTCTTAGCGCCCGCCTCTCTAAATCCCGACGGCTAGAAAACCTGATCGCACAA | |
| | TTACCCGGAGAGAAGAAAAATGGGTTGTTCGGTAACCTTATAGCGCTCTCACTAGGC | |
| | CTGACACCAAATTTTAAGTCGAACTTCGACTTAGCTGAAGATGCCAAATTGCAGCTT | |
| | AGTAAGGACACGTACGATGACGATCTCGACAATCTACTGGCACAAATTGGAGATCAG | |
| | TATGCGGACTTATTTTTGGCTGCCAAAAACCTTAGCGATGCAATCCTCCTATCTGAC | |
| | ATACTGAGAGTTAATACTGAGATTACCAAGGCGCCGTTATCCGCTTCAATGATCAAA | |
| | AGGTACGATGAACATCACCAAGACTTGACACTTCTCAAGGCCCTAGTCCGTCAGCAA | |
| | CTGCCTGAGAAATATAAGGAAATATTCTTTGATCAGTCGAAAAACGGGTACGCAGGT | |
| | TATATTGACGGCGGAGCGAGTCAAGAGGAATTCTACAAGTTTATCAAACCCATATTA | |
| | GAGAAGATGGATGGGACGGAAGAGTTGCTTGTAAAACTCAATCGCGAAGATCTACTG | |
| | CGAAAGCAGCGGACTTTCGACAACGGTAGCATTCCACATCAAATCCACTTAGGCGAA | |
| | TTGCATGCTATACTTAGAAGGCAGGAGGATTTTTATCCGTTCCTCAAAGACAATCGT | |
| | GAAAAGATTGAGAAAATCCTAACCTTTCGCATACCTTACTATGTGGGACCCCTGGCC | |
| | CGAGGGAACTCTCGGTTCGCATGGATGACAAGAAAGTCCGAAGAAACGATTACTCCA | |
| | TGGAATTTTGAGGAAGTTGTCGATAAAGGTGCGTCAGCTCAATCGTTCATCGAGAGG | |
| | ATGACCAACTTTGACAAGAATTTACCGAACGAAAAAGTATTGCCTAAGCACAGTTTA | |
| | CTTTACGAGTATTTCACAGTGTACAATGAACTCACGAAAGTTAAGTATGTCACTGAG | |
| | GGCATGCGTAAACCCGCCTTTCTAAGCGGAGAACAGAAGAAAGCAATAGTAGATCTG | |
| | TTATTCAAGACCAACCGCAAAGTGACAGTTAAGCAATTGAAAGAGGACTACTTTAAG | |
| | AAAATTGAATGCTTCGATTCTGTCGAGATCTCCGGGGTAGAAGATCGATTTAATGCG | |
| | TCACTTGGTACGTATCATGACCTCCTAAAGATAATTAAAGATAAGGACTTCCTGGAT | |
| | AACGAAGAGAATGAAGATATCTTAGAAGATATAGTGTTGACTCTTACCCTCTTTGAA | |
| | GATCGGGAAATGATTGAGGAAAGACTAAAAACATACGCTCACCTGTTCGACGATAAG | |
| | GTTATGAAACAGTTAAAGAGGCGTCGCTATACGGGCTGGGGACGATTGTCGCGGAAA | |
| | CTTATCAACGGGATAAGAGACAAGCAAAGTGGTAAAACTATTCTCGATTTTCTAAAG | |
| | AGCGACGGCTTCGCCAATAGGAACTTTATGCAGCTGATCCATGATGACTCTTTAACC | |
| | TTCAAAGAGGATATACAAAAGGCACAGGTTTCCGGACAAGGGGACTCATTGCACGAA | |
| | CATATTGCGAATCTTGCTGGTTCGCCAGCCATCAAAAAGGGCATACTCCAGACAGTC | |
| | AAAGTAGTGGATGAGCTAGTTAAGGTCATGGGACGTCACAAACCGGAAAACATTGTA | |
| | ATCGAGATGGCACGCGAAAATCAAACGACTCAGAAGGGGCAAAAAAACAGTCGAGAG | |
| | CGGATGAAGAGAATAGAAGAGGGTATTAAAGAACTGGGCAGCCAGATCTTAAAGGAG | |
| | CATCCTGTGGAAAATACCCAATTGCAGAACGAGAAACTTTACCTCTATTACCTACAA | |
| | AATGGAAGGGACATGTATGTTGATCAGGAACTGGACATAAACCGTTTATCTGATTAC | |
| | GACGTCGATCACATTGTACCCCAATCCTTTTTGAAGGACGATTCAATCGACAATAAA | |
| | GTGCTTACACGCTCGGATAAGAACCGAGGGAAAAGTGACAATGTTCCAAGCGAGGAA | |
| | GTCGTAAAGAAAATGAAGAACTATTGGCGGCAGCTCCTAAATGCGAAACTGATAACG | |
| | CAAAGAAAGTTCGATAACTTAACTAAAGCTGAGAGGGGTGGCTTGTCTGAACTTGAC | |
| | AAGGCCGGATTTATTAAACGTCAGCTCGTGGAAACCCGCCAAATCACAAAGCATGTT | |
| | GCACAGATACTAGATTCCCGAATGAATACGAAATACGACGAGAACGATAAGCTGATT | |
| | CGGGAAGTCAAAGTAATCACTTTAAAGTCAAAATTGGTGTCGGACTTCAGAAAGGAT | |
| | TTTCAATTCTATAAAGTTAGGGAGATAAATAACTACCACCATGCGCACGACGCTTAT | |
| | CTTAATGCCGTCGTAGGGACCGCACTCATTAAGAAATACCCGAAGCTAGAAAGTGAG | |
| | TTTGTGTATGGTGATTACAAAGTTTATGACGTCCGTAAGATGATCGCGAAAAGCGAA | |
| | CAGGAGATAGGCAAGGCTACAGCCAAATACTTCTTTTATTCTAACATTATGAATTTC | |
| | TTTAAGACGGAAATCACTCTGGCAAACGGAGAGATACGCAAACGACCTTTAATTGAA | |
| | ACCAATGGGGAGACAGGTGAAATCGTATGGGATAAGGGCCGGGACTTCGCGACGGTG | |
| | AGAAAAGTTTTGTCCATGCCCCAAGTCAACATAGTAAAGAAAACTGAGGTGCAGACC | |
| | GGAGGGTTTTCAAAGGAATCGATTCTTCCAAAAAGGAATAGTGATAAGCTCATCGCT | |
| | CGTAAAAAGGACTGGGACCCGAAAAAGTACGGTGGCTTCGATAGCCCTACAGTTGCC | |
| | TATTCTGTCCTAGTAGTGGCAAAAGTTGAGAAGGGAAAATCCAAGAAACTGAAGTCA | |
| | GTCAAAGAATTATTGGGGATAACGATTATGGAGCGCTCGTCTTTTGAAAAGAACCCC | |
| | ATCGACTTCCTTGAGGCGAAAGGTTACAAGGAAGTAAAAAAGGATCTCATAATTAAA | |
| | CTACCAAAGTATAGTCTGTTTGAGTTAGAAAATGGCCGAAAACGGATGTTGGCTAGC | |
| | GCCGGAGAGCTTCAAAAGGGGAACGAACTCGCACTACCGTCTAAATACGTGAATTTC | |
| | CTGTATTTAGCGTCCCATTACGAGAAGTTGAAAGGTTCACCTGAAGATAACGAACAG | |
| | AAGCAACTTTTTGTTGAGCAGCACAAACATTATCTCGACGAAATCATAGAGCAAATT | |
| | TCGGAATTCAGTAAGAGAGTCATCCTAGCTGATGCCAATCTGGACAAAGTATTAAGC | |
| | GCATACAACAAGCACAGGGATAAACCCATACGTGAGCAGGCGGAAAATATTATCCAT | |
| | TTGTTTACTCTTACCAACCTCGGCGCTCCAGCCGCATTCAAGTATTTTGACACAACG | |
| | ATAGATCGCAAACGATACACTTCTACCAAGGAGGTGCTAGACGCGACACTGATTCAC | |
| | CAATCCATCACGGGATTATATGAAACTCGGATAGATTTGTCACAGCTTGGGGGTGAC | |
| | GGATCCCCCAAGAAGAAGAGGAAAGTCTCGAGCGACTACAAAGACCATGACGGTGAT | |
| | TATAAAGATCATGACATCGATTACAAGGATGACGATGACAAGGCTGCAGGA | |
| SpCas9 | MDKKYSIGLDIGTNSVGWAVITDEYKVPSKKFKVLGNTDRHSIKKNLIGALLFDSGE | SEQ ID NO: 79 |
| Streptococcus | TAEATRLKRTARRRYTRRKNRICYLQEIFSNEMAKVDDSFFHRLEESFLVEEDKKHE | |
| pyogenes | RHPIFGNIVDEVAYHEKYPTIYHLRKKLVDSTDKADLRLIYLALAHMIKFRGHFLIE | |
| wild type | GDLNPDNSDVDKLFIQLVQTYNQLFEENPINASGVDAKAILSARLSKSRRLENLIAQ | |
| Encoded | LPGEKKNGLFGNLIALSLGLTPNFKSNFDLAEDAKLQLSKDTYDDDLDNLLAQIGDQ | |
| product of | YADLFLAAKNLSDAILLSDILRVNTEITKAPLSASMIKRYDEHHQDLTLLKALVRQQ | |
| SWBC2D7W014 | LPEKYKEIFFDQSKNGYAGYIDGGASQEEFYKFIKPILEKMDGTEELLVKLNREDLL | |
| | RKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |
| | GSPKKKRKVSSDYKDHDGDYKDHDIDYKDDDDKAAG | |
| SpCas9 | ATGGATAAGAAATACTCAATAGGCTTAGATATCGGCACAAATAGCGTCGGATGGGCG | SEQ ID NO: 80 |
| Streptococcus | GTGATCACTGATGAATATAAGGTTCCGTCTAAAAAGTTCAAGGTTCTGGGAAATACA | |
| pyogenes | GACCGCCACAGTATCAAAAAAAATCTTATAGGGGCTCTTTTATTTGACAGTGGAGAG | |
| M1GAS wild | ACAGCGGAAGCGACTCGTCTCAAACGGACAGCTCGTAGAAGGTATACACGTCGGAAG | |
| type | AATCGTATTTGTTATCTACAGGAGATTTTTTCAAATGAGATGGCGAAAGTAGATGAT | |
| NC_002737.2 | AGTTTCTTTCATCGACTTGAAGAGTCTTTTTTGGTGGAAGAAGACAAGAAGCATGAA | |
| | CGTCATCCTATTTTTGGAAATATAGTAGATGAAGTTGCTTATCATGAGAAATATCCA | |
| | ACTATCTATCATCTGCGAAAAAAATTGGTAGATTCTACTGATAAAGCGGATTTGCGC | |
| | TTAATCTATTTGGCCTTAGCGCATATGATTAAGTTTCGTGGTCATTTTTTGATTGAG | |
| | GGAGATTTAAATCCTGATAATAGTGATGTGGACAAACTATTTATCCAGTTGGTACAA | |
| | ACCTACAATCAATTATTTGAAGAAAACCCTATTAACGCAAGTGGAGTAGATGCTAAA | |
| | GCGATTCTTTCTGCACGATTGAGTAAATCAAGACGATTAGAAAATCTCATTGCTCAG | |
| | CTCCCCGGTGAGAAGAAAAATGGCTTATTTGGGAATCTCATTGCTTTGTCATTGGGT | |
| | TTGACCCCTAATTTTAAATCAAATTTTGATTTGGCAGAAGATGCTAAATTACAGCTT | |
| | TCAAAAGATACTTACGATGATGATTTAGATAATTTATTGGCGCAAATTGGAGATCAA | |
| | TATGCTGATTTGTTTTTGGCAGCTAAGAATTTATCAGATGCTATTTTACTTTCAGAT | |
| | ATCCTAAGAGTAAATACTGAAATAACTAAGGCTCCCCTATCAGCTTCAATGATTAAA | |
| | CGCTACGATGAACATCATCAAGACTTGACTCTTTTAAAAGCTTTAGTTCGACAACAA | |
| | CTTCCAGAAAAGTATAAAGAAATCTTTTTTGATCAATCAAAAAACGGATATGCAGGT | |
| | TATATTGATGGGGGAGCTAGCCAAGAAGAATTTTATAAATTTATCAAACCAATTTTA | |
| | GAAAAAATGGATGGTACTGAGGAATTATTGGTGAAACTAAATCGTGAAGATTTGCTG | |
| | CGCAAGCAACGGACCTTTGACAACGGCTCTATTCCCCATCAAATTCACTTGGGTGAG | |
| | CTGCATGCTATTTTGAGAAGACAAGAAGACTTTTATCCATTTTTAAAAGACAATCGT | |
| | GAGAAGATTGAAAAAATCTTGACTTTTCGAATTCCTTATTATGTTGGTCCATTGGCG | |
| | CGTGGCAATAGTCGTTTTGCATGGATGACTCGGAAGTCTGAAGAAACAATTACCCCA | |
| | TGGAATTTTGAAGAAGTTGTCGATAAAGGTGCTTCAGCTCAATCATTTATTGAACGC | |
| | ATGACAAACTTTGATAAAAATCTTCCAAATGAAAAAGTACTACCAAAACATAGTTTG | |
| | CTTTATGAGTATTTTACGGTTTATAACGAATTGACAAAGGTCAAATATGTTACTGAA | |
| | GGAATGCGAAAACCAGCATTTCTTTCAGGTGAACAGAAGAAAGCCATTGTTGATTTA | |
| | CTCTTCAAAACAAATCGAAAAGTAACCGTTAAGCAATTAAAAGAAGATTATTTCAAA | |
| | AAAATAGAATGTTTTGATAGTGTTGAAATTTCAGGAGTTGAAGATAGATTTAATGCT | |
| | TCATTAGGTACCTACCATGATTTGCTAAAAATTATTAAAGATAAAGATTTTTTGGAT | |
| | AATGAAGAAAATGAAGATATCTTAGAGGATATTGTTTTAACATTGACCTTATTTGAA | |
| | GATAGGGAGATGATTGAGGAAAGACTTAAAACATATGCTCACCTCTTTGATGATAAG | |
| | GTGATGAAACAGCTTAAACGTCGCCGTTATACTGGTTGGGGACGTTTGTCTCGAAAA | |
| | TTGATTAATGGTATTAGGGATAAGCAATCTGGCAAAACAATATTAGATTTTTTGAAA | |
| | TCAGATGGTTTTGCCAATCGCAATTTTATGCAGCTGATCCATGATGATAGTTTGACA | |
| | TTTAAAGAAGACATTCAAAAAGCACAAGTGTCTGGACAAGGCGATAGTTTACATGAA | |
| | CATATTGCAAATTTAGCTGGTAGCCCTGCTATTAAAAAAGGTATTTTACAGACTGTA | |
| | AAAGTTGTTGATGAATTGGTCAAAGTAATGGGGCGGCATAAGCCAGAAAATATCGTT | |
| | ATTGAAATGGCACGTGAAAATCAGACAACTCAAAAGGGCCAGAAAAATTCGCGAGAG | |
| | CGTATGAAACGAATCGAAGAAGGTATCAAAGAATTAGGAAGTCAGATTCTTAAAGAG | |
| | CATCCTGTTGAAAATACTCAATTGCAAAATGAAAAGCTCTATCTCTATTATCTCCAA | |
| | AATGGAAGAGACATGTATGTGGACCAAGAATTAGATATTAATCGTTTAAGTGATTAT | |
| | GATGTCGATCACATTGTTCCACAAAGTTTCCTTAAAGACGATTCAATAGACAATAAG | |
| | GTCTTAACGCGTTCTGATAAAAATCGTGGTAAATCGGATAACGTTCCAAGTGAAGAA | |
| | GTAGTCAAAAAGATGAAAAACTATTGGAGACAACTTCTAAACGCCAAGTTAATCACT | |
| | CAACGTAAGTTTGATAATTTAACGAAAGCTGAACGTGGAGGTTTGAGTGAACTTGAT | |
| | AAAGCTGGTTTTATCAAACGCCAATTGGTTGAAACTCGCCAAATCACTAAGCATGTG | |
| | GCACAAATTTTGGATAGTCGCATGAATACTAAATACGATGAAAATGATAAACTTATT | |
| | CGAGAGGTTAAAGTGATTACCTTAAAATCTAAATTAGTTTCTGACTTCCGAAAAGAT | |
| | TTCCAATTCTATAAAGTACGTGAGATTAACAATTACCATCATGCCCATGATGCGTAT | |
| | CTAAATGCCGTCGTTGGAACTGCTTTGATTAAGAAATATCCAAAACTTGAATCGGAG | |
| | TTTGTCTATGGTGATTATAAAGTTTATGATGTTCGTAAAATGATTGCTAAGTCTGAG | |
| | CAAGAAATAGGCAAAGCAACCGCAAAATATTTCTTTTACTCTAATATCATGAACTTC | |
| | TTCAAAACAGAAATTACACTTGCAAATGGAGAGATTCGCAAACGCCCTCTAATCGAA | |
| | ACTAATGGGGAAACTGGAGAAATTGTCTGGGATAAAGGGCGAGATTTTGCCACAGTG | |
| | CGCAAAGTATTGTCCATGCCCCAAGTCAATATTGTCAAGAAAACAGAAGTACAGACA | |
| | GGCGGATTCTCCAAGGAGTCAATTTTACCAAAAAGAAATTCGGACAAGCTTATTGCT | |
| | CGTAAAAAAGACTGGGATCCAAAAAAATATGGTGGTTTTGATAGTCCAACGGTAGCT | |
| | TATTCAGTCCTAGTGGTTGCTAAGGTGGAAAAAGGGAAATCGAAGAAGTTAAAATCC | |
| | GTTAAAGAGTTACTAGGGATCACAATTATGGAAAGAAGTTCCTTTGAAAAAAATCCG | |
| | ATTGACTTTTTAGAAGCTAAAGGATATAAGGAAGTTAAAAAAGACTTAATCATTAAA | |
| | CTACCTAAATATAGTCTTTTTGAGTTAGAAAACGGTCGTAAACGGATGCTGGCTAGT | |
| | GCCGGAGAATTACAAAAAGGAAATGAGCTGGCTCTGCCAAGCAAATATGTGAATTTT | |
| | TTATATTTAGCTAGTCATTATGAAAAGTTGAAGGGTAGTCCAGAAGATAACGAACAA | |
| | AAACAATTGTTTGTGGAGCAGCATAAGCATTATTTAGATGAGATTATTGAGCAAATC | |
| | AGTGAATTTTCTAAGCGTGTTATTTTAGCAGATGCCAATTTAGATAAAGTTCTTAGT | |
| | GCATATAACAAACATAGAGACAAACCAATACGTGAACAAGCAGAAAATATTATTCAT | |
| | TTATTTACGTTGACGAATCTTGGAGCTCCCGCTGCTTTTAAATATTTTGATACAACA | |
| | ATTGATCGTAAACGATATACGTCTACAAAAGAAGTTTTAGATGCCACTCTTATCCAT | |
| | CAATCCATCACTGGTCTTTATGAAACACGCATTGATTTGAGTCAGCTAGGAGGTGAC | |
| | TGA | |
| SpCas9 | MDKKYSIGLDIGTNSVGWAVITDEYKVPSKKFKVLGNTDRHSIKKNLIGALLFDSGE | SEQ ID NO: 81 |
| Streptococcus | TAEATRLKRTARRRYTRRKNRICYLQEIFSNEMAKVDDSFFHRLEESFLVEEDKKHE | |
| pyogenes | RHPIFGNIVDEVAYHEKYPTIYHLRKKLVDSTDKADLRLIYLALAHMIKFRGHFLIE | |
| M1GAS wild | GDLNPDNSDVDKLFIQLVQTYNQLFEENPINASGVDAKAILSARLSKSRRLENLIAQ | |
| type | LPGEKKNGLFGNLIALSLGLTPNFKSNFDLAEDAKLQLSKDTYDDDLDNLLAQIGDQ | |
| Encoded | YADLFLAAKNLSDAILLSDILRVNTEITKAPLSASMIKRYDEHHQDLTLLKALVRQQ | |
| product of | LPEKYKEIFFDQSKNGYAGYIDGGASQEEFYKFIKPILEKMDGTEELLVKLNREDLL | |
| NC_002737.2 | RKQRTFDNGSIPHQIHLGELHAILRRQEDFYPFLKDNREKIEKILTFRIPYYVGPLA | |
| (100% | RGNSRFAWMTRKSEETITPWNFEEVVDKGASAQSFIERMTNFDKNLPNEKVLPKHSL | |
| identical to | LYEYFTVYNELTKVKYVTEGMRKPAFLSGEQKKAIVDLLFKTNRKVTVKQLKEDYFK | |
| the canonical | KIECFDSVEISGVEDRFNASLGTYHDLLKIIKDKDFLDNEENEDILEDIVLTLTLFE | |
| Q99ZW2 | DREMIEERLKTYAHLFDDKVMKQLKRRRYTGWGRLSRKLINGIRDKQSGKTILDFLK | |
| wild type) | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |

The base editors described herein may include any of the above SpCas9 sequences, or any variant thereof having at least 80%, at least 85%, at least 90%, at least 95%, or at least 99% sequence identity thereto.

### (ii) Wild Type Cas9 Orthologs

In other embodiments, the Cas9 protein can be a wild type Cas9 ortholog from another bacterial species. For example, the following Cas9 orthologs can be used in connection with the base editor constructs described in this specification. In addition, any variant Cas9 orthologs having at least 80%, at least 85%, at least 90%, at least 95%, or at least 99% sequence identity to any of the below orthologs may also be used with the present base editors.

| Description | Sequence |
|---|---|
| LfCas 9 | MKEYHIGLDIGTSSIGWAVTDSQFKLMRIKGKTAIGVRLFEEGKTAAERRTFRTTRRRLKRRKWRLHYLDEI |
| Lactobacillus | FAPHLQEVDENFLRRLKQSNIHPEDPTKNQAFIGKLLFPDLLKKNERGYPTLIKMRDELPVEQRAHYPVMNI |
| fermentum | YKLREAMINEDRQFDLREVYLAVHHIVKYRGHFLNNASVDKFKVGRIDFDKSFNVLNEAYEELQNGEGSFTI |
| wild type | EPSKVEKIGQLLLDTKMRKLDRQKAVAKLLEVKVADKEETKRNKQIATAMSKLVLGYKADFATVAMANGNEW |
| GenBank: | KIDLSSETSEDEIEKFREELSDAQNDILTEITSLFSQIMLNEIVPNGMSISESMMDRYWTHERQLAEVKEYL |
| SNX31424.11 | ATQPASARKEFDQVYNKYIGQAPKERGFDLEKGLKKILSKKENWKEIDELLKAGDFLPKQRTSANGVIPHQM |
| | HQQELDRIIEKQAKYYPWLATENPATGERDRHQAKYELDQLVSFRIPYYVGPLVTPEVQKATSGAKFAWAKR |
| | KEDGEITPWNLWDKIDRAESAEAFIKRMTVKDTYLLNEDVLPANSLLYQKYNVLNELNNVRVNGRRLSVGIK |
| | QDIYTELFKKKKTVKASDVASLVMAKTRGVNKPSVEGLSDPKKFNSNLATYLDLKSIVGDKVDDNRYQTDLE |
| | NIIEWRSVFEDGEIFADKLTEVEWLTDEQRSALVKKRYKGWGRLSKKLLTGIVDENGQRIIDLMWNTDQNFK |
| | EIVDQPVFKEQIDQLNQKAITNDGMTLRERVESVLDDAYTSPQNKKAIWQVVRVVEDIVKAVGNAPKSISIE |
| | FARNEGNKGEITRSRRTQLQKLFEDQAHELVKDTSLTEELEKAPDLSDRYYFYFTQGGKDMYTGDPINFDEI |
| | STKYDIDHILPQSFVKDNSLDNRVLTSRKENNKKSDQVPAKLYAAKMKPYWNQLLKQGLITQRKFENLTKDV |
| | DQNIKYRSLGFVKRQLVETRQVIKLTANILGSMYQEAGTEIIETRAGLTKQLREEFDLPKVREVNDYHHAVD |
| | AYLTTFAGQYLNRRYPKLRSFFVYGEYMKFKHGSDLKLRNFNFFHELMEGDKSQGKVVDQQTGELITTRDEV |
| | AKSFDRLLNMKYMLVSKEVHDRSDQLYGATIVTAKESGKLTSPIEIKKNRLVDLYGAYTNGTSAFMTIIKFT |
| | GNKPKYKVIGIPTTSAASLKRAGKPGSESYNQELHRIIKSNPKVKKGFEIVVPHVSYGQLIVDGDCKFTLAS |
| | PTVQHPATQLVLSKKSLETISSGYKILKDKPAIANERLIRVFDEVVGQMNRYFTIFDQRSNRQKVADARDKF |
| | LSLPTESKYEGAKKVQVGKTEVITNLLMGLHANATQGDLKVLGLATFGFFQSTTGLSLSEDTMIVYQSPTGL |
| | FERRICLKDI |
| | (SEQ ID NO: 82) |
| SaCas 9 | MDKKYSIGLDIGTNSVGWAVITDEYKVPSKKFKVLGNTDRHSIKKNLIGALLFDSGETAEATRLKRTARRRY |
| Staphylococcus | TRRKNRICYLQEIFSNEMAKVDDSFFHRLEESFLVEEDKKHERHPIFGNIVDEVAYHEKYPTIYHLRKKLVD |
| aureus | STDKADLRLIYLALAHMIKFRGHFLIEGDLNPDNSDVDKLFIQLVQTYNQLFEENPINASGVDAKAILSARL |
| wild type | SKSRRLENLIAQLPGEKKNGLFGNLIALSLGLTPNFKSNFDLAEDAKLQLSKDTYDDDLDNLLAQIGDQYAD |
| GenBank: | LFLAAKNLSDAILLSDILRVNTEITKAPLSASMIKRYDEHHQDLTLLKALVRQQLPEKYKEIFFDQSKNGYA |
| AYD60528.1 | GYIDGGASQEEFYKFIKPILEKMDGTEELLVKLNREDLLRKQRTFDNGSIPHQIHLGELHAILRRQEDFYPF |
| | LKDNREKIEKILTFRIPYYVGPLARGNSRFAWMTRKSEETITPWNFEEVVDKGASAQSFIERMTNFDKNLPN |
| | EKVLPKHSLLYEYFTVYNELTKVKYVTEGMRKPAFLSGEQKKAIVDLLFKTNRKVTVKQLKEDYFKKIECFD |
| | SVEISGVEDRFNASLGTYHDLLKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLIHDDSLTFKEDIQKAQVSGQGDSL |
| | HEHIANLAGSPAIKKGILQTVKVVDELVKVMGRHKPENIVIEMARENQTTQKGQKNSRERMKRIEEGIKELG |
| | SQILKEHPVENTQLQNEKLYLYYLQNGRDMYVDQELDINRLSDYDVDHIVPQSFLKDDSIDNKVLTRSDKNR |
| | GKSDNVPSEEVVKKMKNYWRQLLNAKLITQRKFDNLTKAERGGLSELDKAGFIKRQLVETRQITKHVAQILD |
| | SRMNTKYDENDKLIREVKVITLKSKLVSDFRKDFQFYKVREINNYHHAHDAYLNAVVGTALIKKYPKLESEF |
| | VYGDYKVYDVRKMIAKSEQEIGKATAKYFFYSNIMNFFKTEITLANGEIRKRPLIETNGETGEIVWDKGRDF |
| | ATVRKVLSMPQVNIVKKTEVQTGGFSKESILPKRNSDKLIARKKDWDPKKYGGFDSPTVAYSVLVVAKVEKG |
| | KSKKLKSVKELLGITIMERSSFEKNPIDFLEAKGYKEVKKDLIIKLPKYSLFELENGRKRMLASAGELQKGN |
| | ELALPSKYVNFLYLASHYEKLKGSPEDNEQKQLFVEQHKHYLDEIIEQISEFSKRVILADANLDKVLSAYNK |
| | HRDKPIREQAENIIHLFTLTNLGAPAAFKYFDTTIDRKRYTSTKEVLDATLIHQSITGLYETRIDLSQLGGD |
| | (SEQ ID NO: 83) |
| SaCas 9 | MGKRNYILGLDIGITSVGYGIIDYETRDVIDAGVRLFKEANVENNEGRRSKRGARRLKRRRRHRIQRVKKLL |
| Staphylococcus | FDYNLLTDHSELSGINPYEARVKGLSQKLSEEEFSAALLHLAKRRGVHNVNEVEEDTGNELSTKEQISRNSK |
| aureus | ALEEKYVAELQLERLKKDGEVRGSINRFKTSDYVKEAKQLLKVQKAYHQLDQSFIDTYIDLLETRRTYYEGP |
| | GEGSPFGWKDIKEWYEMLMGHCTYFPEELRSVKYAYNADLYNALNDLNNLVITRDENEKLEYYEKFQIIENV |
| | FKQKKKPTLKQIAKEILVNEEDIKGYRVTSTGKPEFTNLKVYHDIKDITARKEIIENAELLDQIAKILTIYQ |
| | SSEDIQEELTNLNSELTQEEIEQISNLKGYTGTHNLSLKAINLILDELWHTNDNQIAIFNRLKLVPKKVDLS |
| | QQKEIPTTLVDDFILSPVVKRSFIQSIKVINAIIKKYGLPNDIIIELAREKNSKDAQKMINEMQKRNRQTNE |
| | RIEEIIRTTGKENAKYLIEKIKLHDMQEGKCLYSLEAIPLEDLLNNPFNYEVDHIIPRSVSFDNSFNNKVLV |
| | KQEENSKKGNRTPFQYLSSSDSKISYETFKKHILNLAKGKGRISKTKKEYLLEERDINRFSVQKDFINRNLV |
| | DTRYATRGLMNLLRSYFRVNNLDVKVKSINGGFTSFLRRKWKFKKERNKGYKHHAEDALIIANADFIFKEWK |
| | KLDKAKKVMENQMFEEKQAESMPEIETEQEYKEIFITPHQIKHIKDFKDYKYSHRVDKKPNRKLINDTLYST |
| | RKDDKGNTLIVNNLNGLYDKDNDKLKKLINKSPEKLLMYHHDPQTYQKLKLIMEQYGDEKNPLYKYYEETGN |
| | YLTKYSKKDNGPVIKKIKYYGNKLNAHLDITDDYPNSRNKVVKLSLKPYRFDVYLDNGVYKFVTVKNLDVIK |
| | KENYYEVNSKCYEEAKKLKKISNQAEFIASFYKNDLIKINGELYRVIGVNNDLLNRIEVNMIDITYREYLEN |
| | MNDKRPPHIIKTIASKTQSIKKYSTDILGNLYEVKSKKHPQIIKK |
| | (SEQ ID NO: 84) |
| StCas9 | MLFNKCIIISINLDFSNKEKCMTKPYSIGLDIGTNSVGWAVITDNYKVPSKKMKVLGNTSKKYIKKNLLGVL |
| Streptococcus | LFDSGITAEGRRLKRTARRRYTRRRNRILYLQEIFSTEMATLDDAFFQRLDDSFLVPDDKRDSKYPIFGNLV |
| thermophilus | EEKVYHDEFPTIYHLRKYLADSTKKADLRLVYLALAHMIKYRGHFLIEGEFNSKNNDIQKNFQDFLDTYNAI |
| UniProtKB/ | FESDLSLENSKQLEEIVKDKISKLEKKDRILKLFPGEKNSGIFSEFLKLIVGNQADFRKCFNLDEKASLHFS |
| Swiss-Prot: | KESYDEDLETLLGYIGDDYSDVFLKAKKLYDAILLSGFLTVTDNETEAPLSSAMIKRYNEHKEDLALLKEYI |
| G3ECR1.2 | RNISLKTYNEVFKDDTKNGYAGYIDGKTNQEDFYVYLKNLLAEFEGADYFLEKIDREDFLRKQRTFDNGSIP |
| Wild type | YQIHLQEMRAILDKQAKFYPFLAKNKERIEKILTFRIPYYVGPLARGNSDFAWSIRKRNEKITPWNFEDVID |
| | KESSAEAFINRMTSFDLYLPEEKVLPKHSLLYETFNVYNELTKVRFIAESMRDYQFLDSKQKKDIVRLYFKD |
| | KRKVTDKDIIEYLHAIYGYDGIELKGIEKQFNSSLSTYHDLLNIINDKEFLDDSSNEAIIEEIIHTLTIFED |
| | REMIKQRLSKFENIFDKSVLKKLSRRHYTGWGKLSAKLINGIRDEKSGNTILDYLIDDGISNRNFMQLIHDD |
| | ALSFKKKIQKAQIIGDEDKGNIKEVVKSLPGSPAIKKGILQSIKIVDELVKVMGGRKPESIVVEMARENQYT |
| | NQGKSNSQQRLKRLEKSLKELGSKILKENIPAKLSKIDNNALQNDRLYLYYLQNGKDMYTGDDLDIDRLSNY |
| | DIDHIIPQAFLKDNSIDNKVLVSSASNRGKSDDFPSLEVVKKRKTFWYQLLKSKLISQRKFDNLTKAERGGL |
| | LPEDKAGFIQRQLVETRQITKHVARLLDEKFNNKKDENNRAVRTVKIITLKSTLVSQFRKDFELYKVREIND |
| | FHHAHDAYLNAVIASALLKKYPKLEPEFVYGDYPKYNSFRERKSATEKVYFYSNIMNIFKKSISLADGRVIE |
| | RPLIEVNEETGESVWNKESDLATVRRVLSYPQVNVVKKVEEQNHGLDRGKPKGLFNANLSSKPKPNSNENLV |
| | GAKEYLDPKKYGGYAGISNSFAVLVKGTIEKGAKKKITNVLEFQGISILDRINYRKDKLNFLLEKGYKDIEL |
| | IIELPKYSLFELSDGSRRMLASILSTNNKRGEIHKGNQIFLSQKFVKLLYHAKRISNTINENHRKYVENHKK |
| | EFEELFYYILEFNENYVGAKKNGKLLNSAFQSWQNHSIDELCSSFIGPTGSERKGLFELTSRGSAADFEFLG |
| | VKIPRYRDYTPSSLLKDATLIHQSVTGLYETRIDLAKLGEG |
| | (SEQ ID NO: 85) |
| LcCas 9 | MKIKNYNLALTPSTSAVGHVEVDDDLNILEPVHHQKAIGVAKFGEGETAEARRLARSARRTTKRRANRINHY |
| Lactobacillus | FNEIMKPEIDKVDPLMFDRIKQAGLSPLDERKEFRTVIFDRPNIASYYHNQFPTIWHLQKYLMITDEKADIR |
| crispatus | LIYWALHSLLKHRGHFFNTTPMSQFKPGKLNLKDDMLALDDYNDLEGLSFAVANSPEIEKVIKDRSMHKKEK |
| NCBI | IAELKKLIVNDVPDKDLAKRNNKIITQIVNAIMGNSFHLNFIFDMDLDKLTSKAWSFKLDDPELDTKFDAIS |
| Reference | GSMTDNQIGIFETLQKIYSAISLLDILNGSSNVVDAKNALYDKHKRDLNLYFKFLNTLPDEIAKTLKAGYTL |
| Sequence: | YIGNRKKDLLAARKLLKVNVAKNFSQDDFYKLINKELKSIDKQGLQTRFSEKVGELVAQNNFLPVRQRSSDN |
| WP_133478044.1 | VFIPYQLNAITFNKILENQGKYYDFLVKPNPAKKDRKNAPYELSQLMQFTIPYYVGPLVTPEEQVKSGIPKT |
| Wild type | SRFAWMVRKDNGAITPWNFYDKVDIEATADKFIKRSIAKDSYLLSELVLPKHSLLYEKYEVFNELSNVSLDG |
| | KKLSGGVKQILFNEVFKKTNKVNTSRILKALAKHNIPGSKITGLSNPEEFTSSLQTYNAWKKYFPNQIDNFA |
| | YQQDLEKMIEWSTVFEDHKILAKKLDEIEWLDDDQKKFVANTRLRGWGRLSKRLLTGLKDNYGKSIMQRLET |
| | TKANFQQIVYKPEFREQIDKISQAAAKNQSLEDILANSYTSPSNRKAIRKTMSVVDEYIKLNHGKEPDKIFL |
| | MFQRSEQEKGKQTEARSKQLNRILSQLKADKSANKLFSKQLADEFSNAIKKSKYKLNDKQYFYFQQLGRDAL |
| | TGEVIDYDELYKYTVLHIIPRSKLTDDSQNNKVLTKYKIVDGSVALKFGNSYSDALGMPIKAFWTELNRLKL |
| | IPKGKLLNLTTDFSTLNKYQRDGYIARQLVETQQIVKLLATIMQSRFKHTKIIEVRNSQVANIRYQFDYFRI |
| | KNLNEYYRGFDAYLAAVVGTYLYKVYPKARRLFVYGQYLKPKKTNQENQDMHLDSEKKSQGFNFLWNLLYGK |
| | QDQIFVNGTDVIAFNRKDLITKMNTVYNYKSQKISLAIDYHNGAMFKATLFPRNDRDTAKTRKLIPKKKDYD |
| | TDIYGGYTSNVDGYMLLAEIIKRDGNKQYGFYGVPSRLVSELDTLKKTRYTEYEEKLKEIIKPELGVDLKKI |
| | KKIKILKNKVPFNQVIIDKGSKFFITSTSYRWNYRQLILSAESQQTLMDLVVDPDFSNHKARKDARKNADER |
| | LIKVYEEILYQVKNYMPMFVELHRCYEKLVDAQKTFKSLKISDKAMVLNQILILLHSNATSPVLEKLGYHTR |
| | FTLGKKHNLISENAVLVTQSITGLKENHVSIKQML |
| | (SEQ ID NO: 86) |
| PdCas9 | MTNEKYSIGLDIGTSSIGFAVVNDNNRVIRVKGKNAIGVRLFDEGKAAADRRSFRTTRRSFRTTRRRLSRRR |
| Pedicoccus | WRLKLLREIFDAYITPVDEAFFIRLKESNLSPKDSKKQYSGDILFNDRSDKDFYEKYPTIYHLRNALMTEHR |
| damnosus | KFDVREIYLAIHHIMKFRGHFLNATPANNFKVGRLNLEEKFEELNDIYQRVFPDESIEFRTDNLEQIKEVLL |
| NCBI | DNKRSRADRQRTLVSDIYQSSEDKDIEKRNKAVATEILKASLGNKAKLNVITNVEVDKEAAKEWSITFDSES |
| Reference | IDDDLAKIEGQMTDDGHEIIEVLRSLYSGITLSAIVPENHTLSQSMVAKYDLHKDHLKLFKKLINGMTDTKK |
| Sequence: | AKNLRAAYDGYIDGVKGKVLPQEDFYKQVQVNLDDSAEANEIQTYIDQDIFMPKQRTKANGSIPHQLQQQEL |
| WP_062913273.1 | DQIIENQKAYYPWLAELNPNPDKKRQQLAKYKLDELVTFRVPYYVGPMITAKDQKNQSGAEFAWMIRKEPGN |
| Wild type | ITPWNFDQKVDRMATANQFIKRMTTTDTYLLGEDVLPAQSLLYQKFEVLNELNKIRIDHKPISIEQKQQIFN |
| | DLFKQFKNVTIKHLQDYLVSQGQYSKRPLIEGLADEKRFNSSLSTYSDLCGIFGAKLVEENDRQEDLEKIIE |
| | WSTIFEDKKIYRAKLNDLTWLTDDQKEKLATKRYQGWGRLSRKLLVGLKNSEHRNIMDILWITNENFMQIQA |
| | EPDFAKLVTDANKGMLEKTDSQDVINDLYTSPQNKKAIRQILLVVHDIQNAMHGQAPAKIHVEFARGEERNP |
| | RRSVRQRQRQVEAAYEKVSNELVSAKVRQEFKEAINNKRDFKDRLFLYFMQGGIDIYTGKQLNIDQLSSYQI |
| | DHILPQAFVKDDSLTNRVLTNENQVKADSVPIDIFGKKMLSVWGRMKDQGLISKGKYRNLTMNPENISAHTE |
| | NGFINRQLVETRQVIKLAVNILADEYGDSTQIISVKADLSHQMREDFELLKNRDVNDYHHAFDAYLAAFIGN |
| | YLLKRYPKLESYFVYGDFKKFTQKETKMRRFNFIYDLKHCDQVVNKETGEILWTKDEDIKYIRHLFAYKKIL |
| | VSHEVREKRGALYNQTIYKAKDDKGSGQESKKLIRIKDDKETKIYGGYSGKSLAYMTIVQITKKNKVSYRVI |
| | GIPTLALARLNKLENDSTENNGELYKIIKPQFTHYKVDKKNGEIIETTDDFKIVVSKVRFQQLIDDAGQFFM |
| | LASDTYKNNAQQLVISNNALKAINNTNITDCPRDDLERLDNLRLDSAFDEIVKKMDKYFSAYDANNFREKIR |
| | NSNLIFYQLPVEDQWENNKITELGKRTVLTRILQGLHANATTTDMSIFKIKTPFGQLRQRSGISLSENAQLI |
| | YQSPTGLFERRVQLNKIK |
| | (SEQ ID NO: 87) |
| FnCas9 | MKKQKFSDYYLGFDIGTNSVGWCVTDLDYNVLRFNKKDMWGSRLFEEAKTAAERRVRQRNSRRRLKRRKWRL |
| Fusobaterium | NLLEEIFSNEILKIDSNFFRRLKESSLWLEDKSSKEKFTLFNDDNYKDYDFYKQYPTIFHLRNELIKNPEKK |
| nucleatum | DIRLVYLAIHSIFKSRGHFLFEGQNLKEIKNFETLYNNLIAFLEDNGINKIIDKNNIEKLEKIVCDSKKGLK |
| NCBI | DKEKEFKEIFNSDKQLVAIFKLSVGSSVSLNDLFDTDEYKKGEVEKEKISFREQIYEDDKPIYYSILGEKIE |
| Reference | LLDIAKTFYDFMVLNNILADSQYISEAKVKLYEEHKKDLKNLKYIIRKYNKGNYDKLFKDKNENNYSAYIGL |
| Sequence: | NKEKSKKEVIEKSRLKIDDLIKNIKGYLPKVEEIEEKDKAIFNKILNKIELKTILPKQRISDNGTLPYQIHE |
| WP_060798984.1 | AELEKILENQSKYYDFLNYEENGIITKDKLLMTFKFRIPYYVGPLNSYHKDKGGNSWIVRKEEGKILPWNFE |
| | QKVDIEKSAEEFIKRMTNKCTYLNGEDVIPKDTFLYSEYVILNELNKVQVNDEFLNEENKRKIIDELFKENK |
| | KVSEKKFKEYLLVKQIVDGTIELKGVKDSFNSNYISYIRFKDIFGEKLNLDIYKEISEKSILWKCLYGDDKK |
| | IFEKKIKNEYGDILTKDEIKKINTFKFNNWGRLSEKLLTGIEFINLETGECYSSVMDALRRTNYNLMELLSS |
| | KFTLQESINNENKEMNEASYRDLIEESYVSPSLKRAIFQTLKIYEEIRKITGRVPKKVFIEMARGGDESMKN |
| | KKIPARQEQLKKLYDSCGNDIANFSIDIKEMKNSLISYDNNSLRQKKLYLYYLQFGKCMYTGREIDLDRLLQ |
| | NNDTYDIDHIYPRSKVIKDDSFDNLVLVLKNENAEKSNEYPVKKEIQEKMKSFWRFLKEKNFISDEKYKRLT |
| | GKDDFELRGFMARQLVNVRQTTKEVGKILQQIEPEIKIVYSKAEIASSFREMFDFIKVRELNDTHHAKDAYL |
| | NIVAGNVYNTKFTEKPYRYLQEIKENYDVKKIYNYDIKNAWDKENSLEIVKKNMEKNTVNITRFIKEKKGQL |
| | FDLNPIKKGETSNEIISIKPKVYNGKDDKLNEKYGYYKSLNPAYFLYVEHKEKNKRIKSFERVNLVDVNNIK |
| | DEKSLVKYLIENKKLVEPRVIKKVYKRQVILINDYPYSIVTLDSNKLMDFENLKPLFLENKYEKILKNVIKF |
| | LEDNQGKSEENYKFIYLKKKDRYEKNETLESVKDRYNLEFNEMYDKFLEKLDSKDYKNYMNNKKYQELLDVK |
| | EKFIKLNLFDKAFTLKSFLDLFNRKTMADFSKVGLTKYLGKIQKISSNVLSKNELYLLEESVTGLFVKKIKL |
| | (SEQ ID NO: 88) |
| EcCas9 | RRKQRIQILQELLGEEVLKTDPGFFHRMKESRYVVEDKRTLDGKQVELPYALFVDKDYTDKEYYKQFPTINH |
| Enterococcus | LIVYLMTTSDTPDIRLVYLALHYYMKNRGNFLHSGDINNVKDINDILEQLDNVLETFLDGWNLKLKSYVEDI |
| cecorum | KNIYNRDLGRGERKKAFVNTLGAKTKAEKAFCSLISGGSTNLAELFDDSSLKEIETPKIEFASSSLEDKIDG |
| NCBI | IQEALEDRFAVIEAAKRLYDWKTLTDILGDSSSLAEARVNSYQMHHEQLLELKSLVKEYLDRKVFQEVFVSL |
| Reference | NVANNYPAYIGHTKINGKKKELEVKRTKRNDFYSYVKKQVIEPIKKKVSDEAVLTKLSEIESLIEVDKYLPL |
| Sequence: | QVNSDNGVIPYQVKLNELTRIFDNLENRIPVLRENRDKIIKTFKFRIPYYVGSLNGVVKNGKCTNWMVRKEE |
| WP_047338501.1 | GKIYPWNFEDKVDLEASAEQFIRRMTNKCTYLVNEDVLPKYSLLYSKYLVLSELNNLRIDGRPLDVKIKQDI |
| Wild type | YENVFKKNRKVTLKKIKKYLLKEGIITDDDELSGLADDVKSSLTAYRDFKEKLGHLDLSEAQMENIILNITL |
| | FGDDKKLLKKRLAALYPFIDDKSLNRIATLNYRDWGRLSERFLSGITSVDQETGELRTIIQCMYETQANLMQ |
| | LLAEPYHFVEAIEKENPKVDLESISYRIVNDLYVSPAVKRQIWQTLLVIKDIKQVMKHDPERIFIEMAREKQ |
| | ESKKTKSRKQVLSEVYKKAKEYEHLFEKLNSLTEEQLRSKKIYLYFTQLGKCMYSGEPIDFENLVSANSNYD |
| | IDHIYPQSKTIDDSFNNIVLVKKSLNAYKSNHYPIDKNIRDNEKVKTLWNTLVSKGLITKEKYERLIRSTPF |
| | SDEELAGFIARQLVETRQSTKAVAEILSNWFPESEIVYSKAKNVSNFRQDFEILKVRELNDCHHAHDAYLNI |
| | VVGNAYHTKFTNSPYRFIKNKANQEYNLRKLLQKVNKIESNGVVAWVGQSENNPGTIATVKKVIRRNTVLIS |
| | RMVKEVDGQLFDLTLMKKGKGQVPIKSSDERLTDISKYGGYNKATGAYFTFVKSKKRGKVVRSFEYVPLHLS |
| | KQFENNNELLKEYIEKDRGLTDVEILIPKVLINSLFRYNGSLVRITGRGDTRLLLVHEQPLYVSNSFVQQLK |
| | SVSSYKLKKSENDNAKLTKTATEKLSNIDELYDGLLRKLDLPIYSYWFSSIKEYLVESRTKYIKLSIEEKAL |
| | VIFEILHLFQSDAQVPNLKILGLSTKPSRIRIQKNLKDTDKMSIIHQSPSGIFEHEIELTSL |
| | (SEQ ID NO: 89) |
| AhCas9 | MQNGFLGITVSSEQVGWAVTNPKYELERASRKDLWGVRLFDKAETAEDRRMFRTNRRLNQRKKNRIHYLRDI |
| Anaerostipes | FHEEVNQKDPNFFQQLDESNFCEDDRTVEFNFDTNLYKNQFPTVYHLRKYLMETKDKPDIRLVYLAFSKFMK |
| hadrus | NRGHFLYKGNLGEVMDFENSMKGFCESLEKFNIDFPTLSDEQVKEVRDILCDHKIAKTVKKKNIITITKVKS |
| NCBI | KTAKAWIGLFCGCSVPVKVLFQDIDEEIVTDPEKISFEDASYDDYIANIEKGVGIYYEAIVSAKMLFDWSIL |
| Reference | NEILGDHQLLSDAMIAEYNKHHDDLKRLQKIIKGTGSRELYQDIFINDVSGNYVCYVGHAKTMSSADQKQFY |
| Sequence: | TFLKNRLKNVNGISSEDAEWIDTEIKNGTLLPKQTKRDNSVIPHQLQLREFELILDNMQEMYPFLKENREKL |
| WP_044924278.1 | LKIFNFVIPYYVGPLKGVVRKGESTNWMVPKKDGVIHPWNFDEMVDKEASAECFISRMTGNCSYLFNEKVLP |
| Wild type | KNSLLYETFEVLNELNPLKINGEPISVELKQRIYEQLFLTGKKVTKKSLTKYLIKNGYDKDIELSGIDNEFH |
| | SNLKSHIDFEDYDNLSDEEVEQIILRITVFEDKQLLKDYLNREFVKLSEDERKQICSLSYKGWGNLSEMLLN |
| | GITVTDSNGVEVSVMDMLWNTNLNLMQILSKKYGYKAEIEHYNKEHEKTIYNREDLMDYLNIPPAQRRKVNQ |
| | LITIVKSLKKTYGVPNKIFFKISREHQDDPKRTSSRKEQLKYLYKSLKSEDEKHLMKELDELNDHELSNDKV |
| | YLYFLQKGRCIYSGKKLNLSRLRKSNYQNDIDYIYPLSAVNDRSMNNKVLTGIQENRADKYTYFPVDSEIQK |
| | KMKGFWMELVLQGFMTKEKYFRLSRENDFSKSELVSFIEREISDNQQSGRMIASVLQYYFPESKIVFVKEKL |
| | ISSFKRDFHLISSYGHNHLQAAKDAYITIVVGNVYHTKFTMDPAIYFKNHKRKDYDLNRLFLENISRDGQIA |
| | WESGPYGSIQTVRKEYAQNHIAVTKRVVEVKGGLFKQMPLKKGHGEYPLKTNDPRFGNIAQYGGYTNVTGSY |
| | FVLVESMEKGKKRISLEYVPVYLHERLEDDPGHKLLKEYLVDHRKLNHPKILLAKVRKNSLLKIDGFYYRLN |
| | GRSGNALILTNAVELIMDDWQTKTANKISGYMKRRAIDKKARVYQNEFHIQELEQLYDFYLDKLKNGVYKNR |
| | KNNQAELIHNEKEQFMELKTEDQCVLLTEIKKLFVCSPMQADLTLIGGSKHTGMIAMSSNVTKADFAVIAED |
| | PLGLRNKVIYSHKGEK (SEQ ID NO: 90) |
| KvCas9 | MSQNNNKIYNIGLDIGDASVGWAVVDEHYNLLKRHGKHMWGSRLFTQANTAVERRSSRSTRRRYNKRRERIR |
| Kandleria | LLREIMEDMVLDVDPTFFIRLANVSFLDQEDKKDYLKENYHSNYNLFIDKDFNDKTYYDKYPTIYHLRKHLC |
| vitulina | ESKEKEDPRLIYLALHHIVKYRGNFLYEGQKFSMDVSNIEDKMIDVLRQFNEINLFEYVEDRKKIDEVLNVL |
| NCBI | KEPLSKKHKAEKAFALFDTTKDNKAAYKELCAALAGNKFNVTKMLKEAELHDEDEKDISFKFSDATFDDAFV |
| Reference | EKQPLLGDCVEFIDLLHDIYSWVELQNILGSAHTSEPSISAAMIQRYEDHKNDLKLLKDVIRKYLPKKYFEV |
| Sequence: | FRDEKSKKNNYCNYINHPSKTPVDEFYKYIKKLIEKIDDPDVKTILNKIELESFMLKQNSRTNGAVPYQMQL |
| WP_031589969.1 | DELNKILENQSVYYSDLKDNEDKIRSILTFRIPYYFGPLNITKDRQFDWIIKKEGKENERILPWNANEIVDV |
| Wild type | DKTADEFIKRMRNFCTYFPDEPVMAKNSLTVSKYEVLNEINKLRINDHLIKRDMKDKMLHTLFMDHKSISAN |
| | AMKKWLVKNQYFSNTDDIKIEGFQKENACSTSLTPWIDFTKIFGKINESNYDFIEKIIYDVTVFEDKKILRR |
| | RLKKEYDLDEEKIKKILKLKYSGWSRLSKKLLSGIKTKYKDSTRTPETVLEVMERTNMNLMQVINDEKLGFK |
| | KTIDDANSTSVSGKFSYAEVQELAGSPAIKRGIWQALLIVDEIKKIMKHEPAHVYIEFARNEDEKERKDSFV |
| | NQMLKLYKDYDFEDETEKEANKHLKGEDAKSKIRSERLKLYYTQMGKCMYTGKSLDIDRLDTYQVDHIVPQS |
| | LLKDDSIDNKVLVLSSENQRKLDDLVIPSSIRNKMYGFWEKLFNNKIISPKKFYSLIKTEFNEKDQERFINR |
| | QIVETRQITKHVAQIIDNHYENTKVVTVRADLSHQFRERYHIYKNRDINDFHHAHDAYIATILGTYIGHRFE |
| | SLDAKYIYGEYKRIFRNQKNKGKEMKKNNDGFILNSMRNIYADKDTGEIVWDPNYIDRIKKCFYYKDCFVTK |
| | KLEENNGTFFNVTVLPNDTNSDKDNTLATVPVNKYRSNVNKYGGFSGVNSFIVAIKGKKKKGKKVIEVNKLT |
| | GIPLMYKNADEEIKINYLKQAEDLEEVQIGKEILKNQLIEKDGGLYYIVAPTEIINAKQLILNESQTKLVCE |
| | IYKAMKYKNYDNLDSEKIIDLYRLLINKMELYYPEYRKQLVKKFEDRYEQLKVISIEEKCNIIKQILATLHC |
| | NSSIGKIMYSDFKISTTIGRLNGRTISLDDISFIAESPTGMYSKKYKL (SEQ ID NO: 91) |
| EfCas9 | MRLFEEGHTAEDRRLKRTARRRISRRRNRLRYLQAFFEEAMTDLDENFFARLQESFLVPEDKKWHRHPIFAK |
| Enterococcus | LEDEVAYHETYPTIYHLRKKLADSSEQADLRLIYLALAHIVKYRGHFLIEGKLSTENTSVKDQFQQFMVIYN |
| faecalis | QTFVNGESRLVSAPLPESVLIEEELTEKASRTKKSEKVLQQFPQEKANGLFGQFLKLMVGNKADFKKVFGLE |
| NCBI | EEAKITYASESYEEDLEGILAKVGDEYSDVFLAAKNVYDAVELSTILADSDKKSHAKLSSSMIVRFTEHQED |
| Reference | LKKFKRFIRENCPDEYDNLFKNEQKDGYAGYIAHAGKVSQLKFYQYVKKIIQDIAGAEYFLEKIAQENFLRK |
| Sequence: | QRTFDNGVIPHQIHLAELQAIIHRQAAYYPFLKENQEKIEQLVTFRIPYYVGPLSKGDASTFAWLKRQSEEP |
| WP_016631044.1 | IRPWNLQETVDLDQSATAFIERMTNFDTYLPSEKVLPKHSLLYEKFMVFNELTKISYTDDRGIKANFSGKEK |
| Wild type | EKIFDYLFKTRRKVKKKDIIQFYRNEYNTEIVTLSGLEEDQFNASFSTYQDLLKCGLTRAELDHPDNAEKLE |
| | DIIKILTIFEDRQRIRTQLSTFKGQFSAEVLKKLERKHYTGWGRLSKKLINGIYDKESGKTILDYLVKDDGV |
| | SKHYNRNFMQLINDSQLSFKNAIQKAQSSEHEETLSETVNELAGSPAIKKGIYQSLKIVDELVAIMGYAPKR |
| | IVVEMARENQTTSTGKRRSIQRLKIVEKAMAEIGSNLLKEQPTTNEQLRDTRLFLYYMQNGKDMYTGDELSL |
| | HRLSHYDIDHIIPQSFMKDDSLDNLVLVGSTENRGKSDDVPSKEVVKDMKAYWEKLYAAGLISQRKFQRLTK |
| | GEQGGLTLEDKAHFIQRQLVETRQITKNVAGILDQRYNAKSKEKKVQIITLKASLTSQFRSIFGLYKVREVN |
| | DYHHGQDAYLNCVVATTLLKVYPNLAPEFVYGEYPKFQTFKENKATAKAIIYTNLLRFFTEDEPRFTKDGEI |
| | LWSNSYLKTIKKELNYHQMNIVKKVEVQKGGFSKESIKPKGPSNKLIPVKNGLDPQKYGGFDSPVVAYTVLF |
| | THEKGKKPLIKQEILGITIMEKTRFEQNPILFLEEKGFLRPRVLMKLPKYTLYEFPEGRRRLLASAKEAQKG |
| | NQMVLPEHLLTLLYHAKQCLLPNQSESLAYVEQHQPEFQEILERVVDFAEVHTLAKSKVQQIVKLFEANQTA |
| | DVKEIAASFIQLMQFNAMGAPSTFKFFQKDIERARYTSIKEIFDATIIYQSPTGLYETRRKVVD |
| | (SEQ ID NO: 92) |
| Staphylococcus | KRNYILGLDIGITSVGYGIIDYETRDVIDAGVRLFKEANVENNEGRRSKRGARRLKRRRRHRIQRVKKLLFD |
| aureus | YNLLTDHSELSGINPYEARVKGLSQKLSEEEFSAALLHLAKRRGVHNVNEVEEDTGNELSTKEQISRNSKAL |
| Cas9 | EEKYVAELQLERLKKDGEVRGSINRFKTSDYVKEAKQLLKVQKAYHQLDQSFIDTYIDLLETRRTYYEGPGE |
| | GSPFGWKDIKEWYEMLMGHCTYFPEELRSVKYAYNADLYNALNDLNNLVITRDENEKLEYYEKFQIIENVFK |
| | QKKKPTLKQIAKEILVNEEDIKGYRVTSTGKPEFTNLKVYHDIKDITARKEIIENAELLDQIAKILTIYQSS |
| | EDIQEELTNLNSELTQEEIEQISNLKGYTGTHNLSLKAINLILDELWHTNDNQIAIFNRLKLVPKKVDLSQQ |
| | KEIPTTLVDDFILSPVVKRSFIQSIKVINAIIKKYGLPNDIIIELAREKNSKDAQKMINEMQKRNRQTNERI |
| | EEIIRTTGKENAKYLIEKIKLHDMQEGKCLYSLEAIPLEDLLNNPFNYEVDHIIPRSVSFDNSFNNKVLVKQ |
| | EENSKKGNRTPFQYLSSSDSKISYETFKKHILNLAKGKGRISKTKKEYLLEERDINRFSVQKDFINRNLVDT |
| | RYATRGLMNLLRSYFRVNNLDVKVKSINGGFTSFLRRKWKFKKERNKGYKHHAEDALIIANADFIFKEWKKL |
| | DKAKKVMENQMFEEKQAESMPEIETEQEYKEIFITPHQIKHIKDFKDYKYSHRVDKKPNRELINDTLYSTRK |
| | DDKGNTLIVNNLNGLYDKDNDKLKKLINKSPEKLLMYHHDPQTYQKLKLIMEQYGDEKNPLYKYYEETGNYL |
| | TKYSKKDNGPVIKKIKYYGNKLNAHLDITDDYPNSRNKVVKLSLKPYRFDVYLDNGVYKFVTVKNLDVIKKE |
| | NYYEVNSKCYEEAKKLKKISNQAEFIASFYNNDLIKINGELYRVIGVNNDLLNRIEVNMIDITYREYLENMN |
| | DKRPPRIIKTIASKTQSIKKYSTDILGNLYEVKSKKHPQIIKKG |
| | (SEQ ID NO: 93) |
| Geobacillus | MKYKIGLDIGITSIGWAVINLDIPRIEDLGVRIFDRAENPKTGESLALPRRLARSARRRLRRRKHRLERIRR |
| thermodenit- | LFVREGILTKEELNKLFEKKHEIDVWQLRVEALDRKLNNDELARILLHLAKRRGFRSNRKSERTNKENSTML |
| rifleans | KHIEENQSILSSYRTVAEMVVKDPKFSLHKRNKEDNYTNTVARDDLEREIKLIFAKQREYGNIVCTEAFEHE |
| Cas9 | YISIWASQRPFASKDDIEKKVGFCTFEPKEKRAPKATYTFQSFTVWEHINKLRLVSPGGIRALTDDERRLIY |
| | KQAFHKNKITFHDVRTLLNLPDDTRFKGLLYDRNTTLKENEKVRFLELGAYHKIRKAIDSVYGKGAAKSFRP |
| | IDFDTFGYALTMFKDDTDIRSYLRNEYEQNGKRMENLADKVYDEELIEELLNLSFSKFGHLSLKALRNILPY |
| | MEQGEVYSTACERAGYTFTGPKKKQKTVLLPNIPPIANPVVMRALTQARKVVNAIIKKYGSPVSIHIELARE |
| | LSQSFDERRKMQKEQEGNRKKNETAIRQLVEYGLTLNPTGLDIVKFKLWSEQNGKCAYSLQPIEIERLLEPG |
| | YTEVDHVIPYSRSLDDSYTNKVLVLTKENREKGNRTPAEYLGLGSERWQQFETFVLTNKQFSKKKRDRLLRL |
| | HYDENEENEFKNRNLNDTRYISRFLANFIREHLKFADSDDKQKVYTVNGRITAHLRSRWNFNKNREESNLHH |
| | AVDAAIVACTTPSDIARVTAFYQRREQNKELSKKTDPQFPQPWPHFADELQARLSKNPKESIKALNLGNYDN |
| | EKLESLQPVFVSRMPKRSITGAAHQETLRRYIGIDERSGKIQTVVKKKLSEIQLDKTGHFPMYGKESDPRTY |
| | EAIRQRLLEHNNDPKKAFQEPLYKPKKNGELGPIIRTIKIIDTTNQVIPLNDGKTVAYNSNIVRVDVFEKDG |
| | KYYCVPIYTIDMMKGILPNKAIEPNKPYSEWKEMTEDYTFRFSLYPNDLIRIEFPREKTIKTAVGEEIKIKD |
| | LFAYYQTIDSSNGGLSLVSHDNNFSLRSIGSRTLKRFEKYQVDVLGNIYKVRGEKRVGVASSSHSKAGETIR |
| | PL |
| | (SEQ ID NO: 94) |
| ScCas9 | MEKKYSIGLDIGTNSVGWAVITDDYKVPSKKFKVLGNTNRKSIKKNLMGALLFDSGETAEATRLKRTARRRY |
| S. canis | TRRKNRIRYLQEIFANEMAKLDDSFFQRLEESFLVEEDKKNERHPIFGNLADEVAYHRNYPTIYHLRKKLAD |
| 1375 AA | SPEKADLRLIYLALAHIIKFRGHFLIEGKLNAENSDVAKLFYQLIQTYNQLFEESPLDEIEVDAKGILSARL |
| 159.2 kDa | SKSKRLEKLIAVFPNEKKNGLFGNIIALALGLTPNFKSNFDLTEDAKLQLSKDTYDDDLDELLGQIGDQYAD |
| | LFSAAKNLSDAILLSDILRSNSEVTKAPLSASMVKRYDEHHQDLALLKTLVRQQFPEKYAEIFKDDTKNGYA |
| | GYVGIGIKHRKRTTKLATQEEFYKFIKPILEKMDGAEELLAKLNRDDLLRKQRTFDNGSIPHQIHLKELHAI |
| | LRRQEEFYPFLKENREKIEKILTFRIPYYVGPLARGNSRFAWLTRKSEEAITPWNFEEVVDKGASAQSFIER |
| | MTNFDEQLPNKKVLPKHSLLYEYFTVYNELTKVKYVTERMRKPEFLSGEQKKAIVDLLFKTNRKVTVKQLKE |
| | DYFKKIECFDSVEIIGVEDRFNASLGTYHDLLKIIKDKDFLDNEENEDILEDIVLTLTLFEDREMIEERLKT |
| | YAHLFDDKVMKQLKRRHYTGWGRLSRKMINGIRDKQSGKTILDFLKSDGFSNRNFMQLIHDDSLTFKEEIEK |
| | AQVSGQGDSLHEQIADLAGSPAIKKGILQTVKIVDELVKVMGHKPENIVIEMARENQTTTKGLQQSRERKKR |
| | IEEGIKELESQILKENPVENTQLQNEKLYLYYLQNGRDMYVDQELDINRLSDYDVDHIVPQSFIKDDSIDNK |
| | VLTRSVENRGKSDNVPSEEVVKKMKNYWRQLLNAKLITQRKFDNLTKAERGGLSEADKAGFIKRQLVETRQI |
| | TKHVARILDSRMNTKRDKNDKPIREVKVITLKSKLVSDFRKDFQLYKVRDINNYHHAHDAYLNAVVGTALIK |
| | KYPKLESEFVYGDYKVYDVRKMIAKSEQEIGKATAKRFFYSNIMNFFKTEVKLANGEIRKRPLIETNGETGE |
| | VVWNKEKDFATVRKVLAMPQVNIVKKTEVQTGGFSKESILSKRESAKLIPRKKGWDTRKYGGFGSPTVAYSI |
| | LVVAKVEKGKAKKLKSVKVLVGITIMEKGSYEKDPIGFLEAKGYKDIKKELIFKLPKYSLFELENGRRRMLA |
| | SATELQKANELVLPQHLVRLLYYTQNISATTGSNNLGYIEQHREEFKEIFEKIIDFSEKYILKNKVNSNLKS |
| | SFDEQFAVSDSILLSNSFVSLLKYTSFGASGGFTFLDLDVKQGRLRYQTVTEVLDATLIYQSITGLYETRTD |
| | LSQLGGD (SEQ ID NO: 95) |

The base editors described herein may include any of the above Cas9 ortholog sequences, or any variants thereof having at least 80%, at least 85%, at least 90%, at least 95%, or at least 99% sequence identity thereto.

The napDNAbp may include any suitable homologs and/or orthologs or naturally occurring enzymes, such as, Cas9. Cas9 homologs and/or orthologs have been described in various species, including, but not limited to, S. pyogenes and S. therrnophilus. Preferably, the Cas moiety is configured (e.g., mutagenized, recombinantly engineered, or otherwise obtained from nature) as a nickase, i.e., capable of cleaving only a single strand of the target. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase.In some embodiments, the Cas9 protein comprises an amino acid sequence that is at least 80% identical to the amino acid sequence of a Cas9 protein as provided by any one of the variants of Table 3. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the Cas9 orthologs in the above tables.

(iii) Dead Cas9 Variant

In certain embodiments, the base editors described herein may include a dead Cas9, e.g., dead SpCas9, which has no nuclease activity due to one or more mutations that inactive both nuclease domains of Cas9, namely the RuvC domain (which cleaves the non-protospacer DNA strand) and HNH domain (which cleaves the protospacer DNA strand). The nuclease inactivation may be due to one or mutations that result in one or more substitutions and/or deletions in the amino acid sequence of the encoded protein, or any variants thereof having at least 80%, at least 85%, at least 90%, at least 95%, or at least 99% sequence identity thereto.

As used herein, the term “dCas9” refers to a nuclease-inactive Cas9 or nuclease-dead Cas9, or a functional fragment thereof, and embraces any naturally occurring dCas9 from any organism, any naturally-occurring dCas9 equivalent or functional fragment thereof, any dCas9 homolog, ortholog, or paralog from any organism, and any mutant or variant of a dCas9, naturally-occurring or engineered. The term dCas9 is not meant to be particularly limiting and may be referred to as a “dCas9 or equivalent.” Exemplary dCas9 proteins and method for making dCas9 proteins are further described herein and/or are described in the art and are incorporated herein by reference.

In other embodiments, dCas9 corresponds to, or comprises in part or in whole, a Cas9 amino acid sequence having one or more mutations that inactivate the Cas9 nuclease activity. In other embodiments, Cas9 variants having mutations other than D10A and H840A are provided which may result in the full or partial inactivate of the endogenous Cas9 nuclease activity (e.g., nCas9 or dCas9, respectively). Such mutations, by way of example, include other amino acid substitutions at D10 and H820, or other substitutions within the nuclease domains of Cas9 (e.g., substitutions in the HNH nuclease subdomain and/or the RuvC1 subdomain) with reference to a wild type sequence such as Cas9 from Streptococcus pyogenes (NCBI Reference Sequence: NC_017053.1 (SEQ ID NOs: 76-77)). In some embodiments, variants or homologues of Cas9 (e.g., variants of Cas9 from Streptococcus pyogenes (NCBI Reference Sequence: NC_017053.1 (SEQ ID NOs: 76-77))) are provided which are at least about 70% identical, at least about 80% identical, at least about 90% identical, at least about 95% identical, at least about 98% identical, at least about 99% identical, at least about 99.5% identical, or at least about 99.9% identical to NCBI Reference Sequence: NC_017053.1. In some embodiments, variants of dCas9 (e.g., variants of NCBI Reference Sequence: NC_017053.1 (SEQ ID NOs: 76-77)) are provided having amino acid sequences which are shorter, or longer than NC_017053.1 (SEQ ID NOs: 76-77) by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In one embodiment, the dead Cas9 may be based on the canonical SpCas9 sequence of Q99ZW2 and may have the following sequence, which comprises a DlOA and an H810A substitutions (underlined and bolded), or a variant of SEQ ID NO: 74 having at least 80%, at least 85%, at least 90%, at least 95%, or at least 99% sequence identity thereto:

| Description | Sequence | SEQ ID NO: |
|---|---|---|
| dead Cas9 or | MDKKYSIGLXIGTNSVGWAVITDEYKVPSKKFKVLGNTDRHSIKKNLIGALLFDSGE | SEQ ID NO: 100 |
| dCas9 | TAEATRLKRTARRRYTRRKNRICYLQEIFSNEMAKVDDSFFHRLEESFLVEEDKKHE | |
| Streptococcus | RHPIFGNIVDEVAYHEKYPTIYHLRKKLVDSTDKADLRLIYLALAHMIKFRGHFLIE | |
| pyogenes | GDLNPDNSDVDKLFIQLVQTYNQLFEENPINASGVDAKAILSARLSKSRRLENLIAQ | |
| Q99ZW2 Cas9 | LPGEKKNGLFGNLIALSLGLTPNFKSNFDLAEDAKLQLSKDTYDDDLDNLLAQIGDQ | |
| with D10X | YADLFLAAKNLSDAILLSDILRVNTEITKAPLSASMIKRYDEHHQDLTLLKALVRQQ | |
| and H810X | LPEKYKEIFFDQSKNGYAGYIDGGASQEEFYKFIKPILEKMDGTEELLVKLNREDLL | |
| Where “X” is | RKQRTFDNGSIPHQIHLGELHAILRRQEDFYPFLKDNREKIEKILTFRIPYYVGPLA | |
| any amino | RGNSRFAWMTRKSEETITPWNFEEVVDKGASAQSFIERMTNFDKNLPNEKVLPKHSL | |
| acid | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDXIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |
| dead Cas9 or | MDKKYSIGLAIGTNSVGWAVITDEYKVPSKKFKVLGNTDRHSIKKNLIGALLFDSGE | SEQ ID NO: 101 |
| dCas9 | TAEATRLKRTARRRYTRRKNRICYLQEIFSNEMAKVDDSFFHRLEESFLVEEDKKHE | |
| Streptococcus | RHPIFGNIVDEVAYHEKYPTIYHLRKKLVDSTDKADLRLIYLALAHMIKFRGHFLIE | |
| pyogenes | GDLNPDNSDVDKLFIQLVQTYNQLFEENPINASGVDAKAILSARLSKSRRLENLIAQ | |
| Q99ZW2 Cas9 | LPGEKKNGLFGNLIALSLGLTPNFKSNFDLAEDAKLQLSKDTYDDDLDNLLAQIGDQ | |
| with D10A | YADLFLAAKNLSDAILLSDILRVNTEITKAPLSASMIKRYDEHHQDLTLLKALVRQQ | |
| and H810A | LPEKYKEIFFDQSKNGYAGYIDGGASQEEFYKFIKPILEKMDGTEELLVKLNREDLL | |
| | RKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDAIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |

### (iv) Cas9 Nickase Variant

In one embodiment, the base editors described herein comprise a Cas9 nickase. The term “Cas9 nickase” of “nCas9” refers to a variant of Cas9 which is capable of introducing a single-strand break in a double strand DNA molecule target. In some embodiments, the Cas9 nickase comprises only a single functioning nuclease domain. The wild type Cas9 (e.g., the canonical SpCas9) comprises two separate nuclease domains, namely, the RuvC domain (which cleaves the non-protospacer DNA strand) and HNH domain (which cleaves the protospacer DNA strand). In one embodiment, the Cas9 nickase comprises a mutation in the RuvC domain which inactivates the RuvC nuclease activity. For example, mutations in aspartate (D) 10, histidine (H) 983, aspartate (D) 986, or glutamate (E) 762, have been reported as loss-of-function mutations of the RuvC nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the RuvC domain could include D10X, H983X, D986X, or E762X, wherein X is any amino acid other than the wild type amino acid. In certain embodiments, the nickase could be D10A, of H983A, or D986A, or E762A, or a combination thereof. Exemplary nickases include the SpCas9 nickases having the amino acid sequences as set forth in SEQ ID NOs: 102 and 107. In some embodiments, the disclosed base editors and base editor complexes comprise a nickase comprising a sequence having at least 98% or at least 99% identity to SEQ ID NO: 107. In some embodiments, the disclosed base editors and base editor complexes comprise a nickase comprising a sequence having at least 98% or at least 99% identity to SEQ ID NO: 102.

In various embodiments, the Cas9 nickase can having a mutation in the RuvC nuclease domain and have one of the following amino acid sequences, or a variant thereof having an amino acid sequence that has at least 80%, at least 85%, at least 90%, at least 95%, or at least 99% sequence identity thereto.

| Description | Sequence | SEQ ID NO: |
|---|---|---|
| Cas9 nickase | MDKKYSIGLXIGTNSVGWAVITDEYKVPSKKFKVLGNTDRHSIKKNLIGALLFDSGE | SEQ ID NO: 98 |
| Streptococcus | TAEATRLKRTARRRYTRRKNRICYLQEIFSNEMAKVDDSFFHRLEESFLVEEDKKHE | |
| pyogenes | RHPIFGNIVDEVAYHEKYPTIYHLRKKLVDSTDKADLRLIYLALAHMIKFRGHFLIE | |
| Q99ZW2 Cas9 | GDLNPDNSDVDKLFIQLVQTYNQLFEENPINASGVDAKAILSARLSKSRRLENLIAQ | |
| with D10X, | LPGEKKNGLFGNLIALSLGLTPNFKSNFDLAEDAKLQLSKDTYDDDLDNLLAQIGDQ | |
| wherein X is | YADLFLAAKNLSDAILLSDILRVNTEITKAPLSASMIKRYDEHHQDLTLLKALVRQQ | |
| any | LPEKYKEIFFDQSKNGYAGYIDGGASQEEFYKFIKPILEKMDGTEELLVKLNREDLL | |
| alternate | RKQRTFDNGSIPHQIHLGELHAILRRQEDFYPFLKDNREKIEKILTFRIPYYVGPLA | |
| amino acid | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLFDSGE | SEQ ID NO: 99 |
| Streptococcus | TAEATRLKRTARRRYTRRKNRICYLQEIFSNEMAKVDDSFFHRLEESFLVEEDKKHE | |
| pyogenes | RHPIFGNIVDEVAYHEKYPTIYHLRKKLVDSTDKADLRLIYLALAHMIKFRGHFLIE | |
| Q99ZW2 Cas9 | GDLNPDNSDVDKLFIQLVQTYNQLFEENPINASGVDAKAILSARLSKSRRLENLIAQ | |
| with E762X, | LPGEKKNGLFGNLIALSLGLTPNFKSNFDLAEDAKLQLSKDTYDDDLDNLLAQIGDQ | |
| wherein X is | YADLFLAAKNLSDAILLSDILRVNTEITKAPLSASMIKRYDEHHQDLTLLKALVRQQ | |
| any | LPEKYKEIFFDQSKNGYAGYIDGGASQEEFYKFIKPILEKMDGTEELLVKLNREDLL | |
| alternate | RKQRTFDNGSIPHQIHLGELHAILRRQEDFYPFLKDNREKIEKILTFRIPYYVGPLA | |
| amino acid | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIXMARENQTTQKGQKNSRERMKRIEEGIKELGSQILKE | |
| | HPVENTQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLFDSGE | SEQ ID NO: |
| Streptococcus | TAEATRLKRTARRRYTRRKNRICYLQEIFSNEMAKVDDSFFHRLEESFLVEEDKKHE | 100 |
| pyogenes | RHPIFGNIVDEVAYHEKYPTIYHLRKKLVDSTDKADLRLIYLALAHMIKFRGHFLIE | |
| Q99ZW2 Cas9 | GDLNPDNSDVDKLFIQLVQTYNQLFEENPINASGVDAKAILSARLSKSRRLENLIAQ | |
| with H983X, | LPGEKKNGLFGNLIALSLGLTPNFKSNFDLAEDAKLQLSKDTYDDDLDNLLAQIGDQ | |
| wherein X is | YADLFLAAKNLSDAILLSDILRVNTEITKAPLSASMIKRYDEHHQDLTLLKALVRQQ | |
| any | LPEKYKEIFFDQSKNGYAGYIDGGASQEEFYKFIKPILEKMDGTEELLVKLNREDLL | |
| alternate | RKQRTFDNGSIPHQIHLGELHAILRRQEDFYPFLKDNREKIEKILTFRIPYYVGPLA | |
| amino acid | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHXAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLFDSGE | SEQ ID NO: |
| Streptococcus | TAEATRLKRTARRRYTRRKNRICYLQEIFSNEMAKVDDSFFHRLEESFLVEEDKKHE | 101 |
| pyogenes | RHPIFGNIVDEVAYHEKYPTIYHLRKKLVDSTDKADLRLIYLALAHMIKFRGHFLIE | |
| Q99ZW2 Cas9 | GDLNPDNSDVDKLFIQLVQTYNQLFEENPINASGVDAKAILSARLSKSRRLENLIAQ | |
| with D986X, | LPGEKKNGLFGNLIALSLGLTPNFKSNFDLAEDAKLQLSKDTYDDDLDNLLAQIGDQ | |
| wherein X is | YADLFLAAKNLSDAILLSDILRVNTEITKAPLSASMIKRYDEHHQDLTLLKALVRQQ | |
| any | LPEKYKEIFFDQSKNGYAGYIDGGASQEEFYKFIKPILEKMDGTEELLVKLNREDLL | |
| alternate | RKQRTFDNGSIPHQIHLGELHAILRRQEDFYPFLKDNREKIEKILTFRIPYYVGPLA | |
| amino acid | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHXAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |
| Cas9 nickase | MDKKYSIGLAIGTNSVGWAVITDEYKVPSKKFKVLGNTDRHSIKKNLIGALLFDSGE | SEQ ID NO: |
| Streptococcus | TAEATRLKRTARRRYTRRKNRICYLQEIFSNEMAKVDDSFFHRLEESFLVEEDKKHE | 102 |
| pyogenes | RHPIFGNIVDEVAYHEKYPTIYHLRKKLVDSTDKADLRLIYLALAHMIKFRGHFLIE | |
| Q99ZW2 Cas9 | GDLNPDNSDVDKLFIQLVQTYNQLFEENPINASGVDAKAILSARLSKSRRLENLIAQ | |
| with D10A | LPGEKKNGLFGNLIALSLGLTPNFKSNFDLAEDAKLQLSKDTYDDDLDNLLAQIGDQ | |
| | YADLFLAAKNLSDAILLSDILRVNTEITKAPLSASMIKRYDEHHQDLTLLKALVRQQ | |
| | LPEKYKEIFFDQSKNGYAGYIDGGASQEEFYKFIKPILEKMDGTEELLVKLNREDLL | |
| | RKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLFDSGE | SEQ ID NO: |
| Streptococcus | TAEATRLKRTARRRYTRRKNRICYLQEIFSNEMAKVDDSFFHRLEESFLVEEDKKHE | 103 |
| pyogenes | RHPIFGNIVDEVAYHEKYPTIYHLRKKLVDSTDKADLRLIYLALAHMIKFRGHFLIE | |
| Q99ZW2 Cas9 | GDLNPDNSDVDKLFIQLVQTYNQLFEENPINASGVDAKAILSARLSKSRRLENLIAQ | |
| with E762A | LPGEKKNGLFGNLIALSLGLTPNFKSNFDLAEDAKLQLSKDTYDDDLDNLLAQIGDQ | |
| | YADLFLAAKNLSDAILLSDILRVNTEITKAPLSASMIKRYDEHHQDLTLLKALVRQQ | |
| | LPEKYKEIFFDQSKNGYAGYIDGGASQEEFYKFIKPILEKMDGTEELLVKLNREDLL | |
| | RKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIAMARENQTTQKGQKNSRERMKRIEEGIKELGSQILKE | |
| | HPVENTQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLFDSGE | SEQ ID NO: |
| Streptococcus | TAEATRLKRTARRRYTRRKNRICYLQEIFSNEMAKVDDSFFHRLEESFLVEEDKKHE | 104 |
| pyogenes | RHPIFGNIVDEVAYHEKYPTIYHLRKKLVDSTDKADLRLIYLALAHMIKFRGHFLIE | |
| Q99ZW2 Cas9 | GDLNPDNSDVDKLFIQLVQTYNQLFEENPINASGVDAKAILSARLSKSRRLENLIAQ | |
| with H983A | LPGEKKNGLFGNLIALSLGLTPNFKSNFDLAEDAKLQLSKDTYDDDLDNLLAQIGDQ | |
| | YADLFLAAKNLSDAILLSDILRVNTEITKAPLSASMIKRYDEHHQDLTLLKALVRQQ | |
| | LPEKYKEIFFDQSKNGYAGYIDGGASQEEFYKFIKPILEKMDGTEELLVKLNREDLL | |
| | RKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHAAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLFDSGE | SEQ ID NO: |
| Streptococcus | TAEATRLKRTARRRYTRRKNRICYLQEIFSNEMAKVDDSFFHRLEESFLVEEDKKHE | 105 |
| pyogenes | RHPIFGNIVDEVAYHEKYPTIYHLRKKLVDSTDKADLRLIYLALAHMIKFRGHFLIE | |
| Q99ZW2 Cas9 | GDLNPDNSDVDKLFIQLVQTYNQLFEENPINASGVDAKAILSARLSKSRRLENLIAQ | |
| with D986A | LPGEKKNGLFGNLIALSLGLTPNFKSNFDLAEDAKLQLSKDTYDDDLDNLLAQIGDQ | |
| | YADLFLAAKNLSDAILLSDILRVNTEITKAPLSASMIKRYDEHHQDLTLLKALVRQQ | |
| | LPEKYKEIFFDQSKNGYAGYIDGGASQEEFYKFIKPILEKMDGTEELLVKLNREDLL | |
| | RKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHAAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |

In another embodiment, the Cas9 nickase comprises a mutation in the HNH domain which inactivates the HNH nuclease activity. For example, mutations in histidine (H) 840 or asparagine (R) 863 have been reported as loss-of-function mutations of the HNH nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the HNH domain could include H840X and R863X, wherein X is any amino acid other than the wild type amino acid. In certain embodiments, the nickase could be H840A or R863A or a combination thereof.

In various embodiments, the Cas9 nickase can have a mutation in the HNH nuclease domain and have one of the following amino acid sequences, or a variant thereof having an amino acid sequence that has at least 80%, at least 85%, at least 90%, at least 95%, or at least 99% sequence identity thereto.

| Description | Sequence | SEQ ID NO: |
|---|---|---|
| Cas9 nickase | MDKKYSIGLDIGTNSVGWAVITDEYKVPSKKFKVLGNTDRHSIKKNLIGALLFDSGE | SEQ ID NO: |
| Streptococcus | TAEATRLKRTARRRYTRRKNRICYLQEIFSNEMAKVDDSFFHRLEESFLVEEDKKHE | 106 |
| pyogenes | RHPIFGNIVDEVAYHEKYPTIYHLRKKLVDSTDKADLRLIYLALAHMIKFRGHFLIE | |
| Q99ZW2 Cas9 | GDLNPDNSDVDKLFIQLVQTYNQLFEENPINASGVDAKAILSARLSKSRRLENLIAQ | |
| with H840X, | LPGEKKNGLFGNLIALSLGLTPNFKSNFDLAEDAKLQLSKDTYDDDLDNLLAQIGDQ | |
| wherein X is | YADLFLAAKNLSDAILLSDILRVNTEITKAPLSASMIKRYDEHHQDLTLLKALVRQQ | |
| any | LPEKYKEIFFDQSKNGYAGYIDGGASQEEFYKFIKPILEKMDGTEELLVKLNREDLL | |
| alternate | RKQRTFDNGSIPHQIHLGELHAILRRQEDFYPFLKDNREKIEKILTFRIPYYVGPLA | |
| amino acid | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDXIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLFDSGE | SEQ ID NO: |
| Streptococcus | TAEATRLKRTARRRYTRRKNRICYLQEIFSNEMAKVDDSFFHRLEESFLVEEDKKHE | 107 |
| pyogenes | RHPIFGNIVDEVAYHEKYPTIYHLRKKLVDSTDKADLRLIYLALAHMIKFRGHFLIE | |
| Q99ZW2 Cas9 | GDLNPDNSDVDKLFIQLVQTYNQLFEENPINASGVDAKAILSARLSKSRRLENLIAQ | |
| with H840A | LPGEKKNGLFGNLIALSLGLTPNFKSNFDLAEDAKLQLSKDTYDDDLDNLLAQIGDQ | |
| | YADLFLAAKNLSDAILLSDILRVNTEITKAPLSASMIKRYDEHHQDLTLLKALVRQQ | |
| | LPEKYKEIFFDQSKNGYAGYIDGGASQEEFYKFIKPILEKMDGTEELLVKLNREDLL | |
| | RKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDAIVPQSFLKDDSIDNK | |
| | VLTRSDKNRGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLFDSGE | SEQ ID NO: |
| Streptococcus | TAEATRLKRTARRRYTRRKNRICYLQEIFSNEMAKVDDSFFHRLEESFLVEEDKKHE | 108 |
| pyogenes | RHPIFGNIVDEVAYHEKYPTIYHLRKKLVDSTDKADLRLIYLALAHMIKFRGHFLIE | |
| Q99ZW2 Cas9 | GDLNPDNSDVDKLFIQLVQTYNQLFEENPINASGVDAKAILSARLSKSRRLENLIAQ | |
| with R863X, | LPGEKKNGLFGNLIALSLGLTPNFKSNFDLAEDAKLQLSKDTYDDDLDNLLAQIGDQ | |
| wherein X is | YADLFLAAKNLSDAILLSDILRVNTEITKAPLSASMIKRYDEHHQDLTLLKALVRQQ | |
| any | LPEKYKEIFFDQSKNGYAGYIDGGASQEEFYKFIKPILEKMDGTEELLVKLNREDLL | |
| alternate | RKQRTFDNGSIPHQIHLGELHAILRRQEDFYPFLKDNREKIEKILTFRIPYYVGPLA | |
| amino acid | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDHIVPQSFLKDDSIDNK | |
| | VLTRSDKNXGKSDNVPSEEWKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLFDSGE | SEQ ID NO: |
| Streptococcus | TAEATRLKRTARRRYTRRKNRICYLQEIFSNEMAKVDDSFFHRLEESFLVEEDKKHE | 109 |
| pyogenes | RHPIFGNIVDEVAYHEKYPTIYHLRKKLVDSTDKADLRLIYLALAHMIKFRGHFLIE | |
| Q99ZW2 Cas9 | GDLNPDNSDVDKLFIQLVQTYNQLFEENPINASGVDAKAILSARLSKSRRLENLIAQ | |
| with R863A | LPGEKKNGLFGNLIALSLGLTPNFKSNFDLAEDAKLQLSKDTYDDDLDNLLAQIGDQ | |
| | YADLFLAAKNLSDAILLSDILRVNTEITKAPLSASMIKRYDEHHQDLTLLKALVRQQ | |
| | LPEKYKEIFFDQSKNGYAGYIDGGASQEEFYKFIKPILEKMDGTEELLVKLNREDLL | |
| | RKQRTFDNGSIPHQIHLGELHAILRRQEDFYPFLKDNREKIEKILTFRIPYYVGPLA | |
| | RGNSRFAWMTRKSEETITPWNFEEVVDKGASAQSFIERMTNFDKNLPNEKVLPKHSL | |
| | LYEYFTVYNELTKVKYVTEGMRKPAFLSGEQKKAIVDLLFKTNRKVTVKQLKEDYFK | |
| | KIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTV | |
| | KVVDELVKVMGRHKPENIVIEMARENQTTQKGQKNSRERMKRIEEGIKELGSQILKE | |
| | HPVENTQLQNEKLYLYYLQNGRDMYVDQELDINRLSDYDVDHIVPQSFLKDDSIDNK | |
| | VLTRSDKNAGKSDNVPSEEVVKKMKNYWRQLLNAKLITQRKFDNLTKAERGGLSELD | |
| | KAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKSE | |
| | QEIGKATAKYFFYSNIMNFFKTEITLANGEIRKRPLIETNGETGEIVWDKGRDFATV | |
| | RKVLSMPQVNIVKKTEVQTGGFSKESILPKRNSDKLIARKKDWDPKKYGGFDSPTVA | |
| | YSVLVVAKVEKGKSKKLKSVKELLGITIMERSSFEKNPIDFLEAKGYKEVKKDLIIK | |
| | LPKYSLFELENGRKRMLASAGELQKGNELALPSKYVNFLYLASHYEKLKGSPEDNEQ | |
| | KQLFVEQHKHYLDEIIEQISEFSKRVILADANLDKVLSAYNKHRDKPIREQAENIIH | |
| | LFTLTNLGAPAAFKYFDTTIDRKRYTSTKEVLDATLIHQSITGLYETRIDLSQLGGD | |

In some embodiments, the N-terminal methionine is removed from a Cas9 nickase, or from any Cas9 variant, ortholog, or equivalent disclosed or contemplated herein. For example, methionine-minus Cas9 nickases include the following sequences, or a variant thereof having an amino acid sequence that has at least 80%, at least 85%, at least 90%, at least 95%, or at least 99% sequence identity thereto.

| Description | Sequence |
|---|---|
| Cas9 nickase | DKKYSIGLDIGTNSVGWAVITDEYKVPSKKFKVLGNTDRHSIKKNLIGALLFDSGETAEATRLKRTARRR |
| (Met minus) | YTRRKNRICYLQEIFSNEMAKVDDSFFHRLEESFLVEEDKKHERHPIFGNIVDEVAYHEKYPTIYHLRKK |
| Streptococcus | LVDSTDKADLRLIYLALAHMIKFRGHFLIEGDLNPDNSDVDKLFIQLVQTYNQLFEENPINASGVDAKAI |
| pyogenes | LSARLSKSRRLENLIAQLPGEKKNGLFGNLIALSLGLTPNFKSNFDLAEDAKLQLSKDTYDDDLDNLLAQ |
| Q99ZW2 Cas9 | IGDQYADLFLAAKNLSDAILLSDILRVNTEITKAPLSASMIKRYDEHHQDLTLLKALVRQQLPEKYKEIF |
| with H840X, | FDQSKNGYAGYIDGGASQEEFYKFIKPILEKMDGTEELLVKLNREDLLRKQRTFDNGSIPHQIHLGELHA |
| wherein X is | ILRRQEDFYPFLKDNREKIEKILTFRIPYYVGPLARGNSRFAWMTRKSEETITPWNFEEVVDKGASAQSF |
| any | IERMTNFDKNLPNEKVLPKHSLLYEYFTVYNELTKVKYVTEGMRKPAFLSGEQKKAIVDLLFKTNRKVTV |
| alternate | KQLKEDYFKKIECFDSVEISGVEDRFNASLGTYHDLLKIIKDKDFLDNEENEDILEDIVLTLTLFEDREM |
| amino acid | IEERLKTYAHLFDDKVMKQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRHKPENIVIEMARENQTTQ |
| | KGQKNSRERMKRIEEGIKELGSQILKEHPVENTQLQNEKLYLYYLQNGRDMYVDQELDINRLSDYDVDXI |
| | VPQSFLKDDSIDNKVLTRSDKNRGKSDNVPSEEVVKKMKNYWRQLLNAKLITQRKFDNLTKAERGGLSEL |
| | DKAGFIKRQLVETRQITKHVAQILDSRMNTKYDENDKLIREVKVITLKSKLVSDFRKDFQFYKVREINNY |
| | HHAHDAYLNAVVGTALIKKYPKLESEFVYGDYKVYDVRKMIAKSEQEIGKATAKYFFYSNIMNFFKTEIT |
| | LANGEIRKRPLIETNGETGEIVWDKGRDFATVRKVLSMPQVNIVKKTEVQTGGFSKESILPKRNSDKLIA |
| | RKKDWDPKKYGGFDSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDTTI |
| | DRKRYTSTKEVLDATLIHQSITGLYETRIDLSQLGGD (SEQ ID NO: 110) |
| Cas9 nickase | DKKYSIGLDIGTNSVGWAVITDEYKVPSKKFKVLGNTDRHSIKKNLIGALLFDSGETAEATRLKRTARRR |
| Met minus) | YTRRKNRICYLQEIFSNEMAKVDDSFFHRLEESFLVEEDKKHERHPIFGNIVDEVAYHEKYPTIYHLRKK |
| Streptococcus | LVDSTDKADLRLIYLALAHMIKFRGHFLIEGDLNPDNSDVDKLFIQLVQTYNQLFEENPINASGVDAKAI |
| pyogenes | LSARLSKSRRLENLIAQLPGEKKNGLFGNLIALSLGLTPNFKSNFDLAEDAKLQLSKDTYDDDLDNLLAQ |
| Q99ZW2 Cas9 | IGDQYADLFLAAKNLSDAILLSDILRVNTEITKAPLSASMIKRYDEHHQDLTLLKALVRQQLPEKYKEIF |
| with H840A | FDQSKNGYAGYIDGGASQEEFYKFIKPILEKMDGTEELLVKLNREDLLRKQRTFDNGSIPHQIHLGELHA |
| | ILRRQEDFYPFLKDNREKIEKILTFRIPYYVGPLARGNSRFAWMTRKSEETITPWNFEEVVDKGASAQSF |
| | IERMTNFDKNLPNEKVLPKHSLLYEYFTVYNELTKVKYVTEGMRKPAFLSGEQKKAIVDLLFKTNRKVTV |
| | KQLKEDYFKKIECFDSVEISGVEDRFNASLGTYHDLLKIIKDKDFLDNEENEDILEDIVLTLTLFEDREM |
| | IEERLKTYAHLFDDKVMKQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRHKPENIVIEMARENQTTQ |
| | KGQKNSRERMKRIEEGIKELGSQILKEHPVENTQLQNEKLYLYYLQNGRDMYVDQELDINRLSDYDVDAI |
| | VPQSFLKDDSIDNKVLTRSDKNRGKSDNVPSEEVVKKMKNYWRQLLNAKLITQRKFDNLTKAERGGLSEL |
| | DKAGFIKRQLVETRQITKHVAQILDSRMNTKYDENDKLIREVKVITLKSKLVSDFRKDFQFYKVREINNY |
| | HHAHDAYLNAVVGTALIKKYPKLESEFVYGDYKVYDVRKMIAKSEQEIGKATAKYFFYSNIMNFFKTEIT |
| | LANGEIRKRPLIETNGETGEIVWDKGRDFATVRKVLSMPQVNIVKKTEVQTGGFSKESILPKRNSDKLIA |
| | RKKDWDPKKYGGFDSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDTTI |
| | DRKRYTSTKEVLDATLIHQSITGLYETRIDLSQLGGD (SEQ ID NO: 111) |
| Cas9 nickase | DKKYSIGLDIGTNSVGWAVITDEYKVPSKKFKVLGNTDRHSIKKNLIGALLFDSGETAEATRLKRTARRR |
| (Met minus) | YTRRKNRICYLQEIFSNEMAKVDDSFFHRLEESFLVEEDKKHERHPIFGNIVDEVAYHEKYPTIYHLRKK |
| Streptococcus | LVDSTDKADLRLIYLALAHMIKFRGHFLIEGDLNPDNSDVDKLFIQLVQTYNQLFEENPINASGVDAKAI |
| pyogenes | LSARLSKSRRLENLIAQLPGEKKNGLFGNLIALSLGLTPNFKSNFDLAEDAKLQLSKDTYDDDLDNLLAQ |
| Q99ZW2 Cas9 | IGDQYADLFLAAKNLSDAILLSDILRVNTEITKAPLSASMIKRYDEHHQDLTLLKALVRQQLPEKYKEIF |
| with R863X, | FDQSKNGYAGYIDGGASQEEFYKFIKPILEKMDGTEELLVKLNREDLLRKQRTFDNGSIPHQIHLGELHA |
| wherein X is | ILRRQEDFYPFLKDNREKIEKILTFRIPYYVGPLARGNSRFAWMTRKSEETITPWNFEEVVDKGASAQSF |
| any | IERMTNFDKNLPNEKVLPKHSLLYEYFTVYNELTKVKYVTEGMRKPAFLSGEQKKAIVDLLFKTNRKVTV |
| alternate | KQLKEDYFKKIECFDSVEISGVEDRFNASLGTYHDLLKIIKDKDFLDNEENEDILEDIVLTLTLFEDREM |
| amino acid | IEERLKTYAHLFDDKVMKQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRHKPENIVIEMARENQTTQ |
| | KGQKNSRERMKRIEEGIKELGSQILKEHPVENTQLQNEKLYLYYLQNGRDMYVDQELDINRLSDYDVDHI |
| | VPQSFLKDDSIDNKVLTRSDKNXGKSDNVPSEEVVKKMKNYWRQLLNAKLITQRKFDNLTKAERGGLSEL |
| | DKAGFIKRQLVETRQITKHVAQILDSRMNTKYDENDKLIREVKVITLKSKLVSDFRKDFQFYKVREINNY |
| | HHAHDAYLNAVVGTALIKKYPKLESEFVYGDYKVYDVRKMIAKSEQEIGKATAKYFFYSNIMNFFKTEIT |
| | LANGEIRKRPLIETNGETGEIVWDKGRDFATVRKVLSMPQVNIVKKTEVQTGGFSKESILPKRNSDKLIA |
| | RKKDWDPKKYGGFDSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDTTI |
| | DRKRYTSTKEVLDATLIHQSITGLYETRIDLSQLGGD(SEQ ID NO: 112) |
| Cas9 nickase | DKKYSIGLDIGTNSVGWAVITDEYKVPSKKFKVLGNTDRHSIKKNLIGALLFDSGETAEATRLKRTARRR |
| (Met minus) | YTRRKNRICYLQEIFSNEMAKVDDSFFHRLEESFLVEEDKKHERHPIFGNIVDEVAYHEKYPTIYHLRKK |
| Streptococcus | LVDSTDKADLRLIYLALAHMIKFRGHFLIEGDLNPDNSDVDKLFIQLVQTYNQLFEENPINASGVDAKAI |
| pyogenes | LSARLSKSRRLENLIAQLPGEKKNGLFGNLIALSLGLTPNFKSNFDLAEDAKLQLSKDTYDDDLDNLLAQ |
| Q99ZW2 Cas9 | IGDQYADLFLAAKNLSDAILLSDILRVNTEITKAPLSASMIKRYDEHHQDLTLLKALVRQQLPEKYKEIF |
| with R863A | FDQSKNGYAGYIDGGASQEEFYKFIKPILEKMDGTEELLVKLNREDLLRKQRTFDNGSIPHQIHLGELHA |
| | ILRRQEDFYPFLKDNREKIEKILTFRIPYYVGPLARGNSRFAWMTRKSEETITPWNFEEVVDKGASAQSF |
| | IERMTNFDKNLPNEKVLPKHSLLYEYFTVYNELTKVKYVTEGMRKPAFLSGEQKKAIVDLLFKTNRKVTV |
| | KQLKEDYFKKIECFDSVEISGVEDRFNASLGTYHDLLKIIKDKDFLDNEENEDILEDIVLTLTLFEDREM |
| | IEERLKTYAHLFDDKVMKQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRHKPENIVIEMARENQTTQ |
| | KGQKNSRERMKRIEEGIKELGSQILKEHPVENTQLQNEKLYLYYLQNGRDMYVDQELDINRLSDYDVDHI |
| | VPQSFLKDDSIDNKVLTRSDKNAGKSDNVPSEEVVKKMKNYWRQLLNAKLITQRKFDNLTKAERGGLSEL |
| | DKAGFIKRQLVETRQITKHVAQILDSRMNTKYDENDKLIREVKVITLKSKLVSDFRKDFQFYKVREINNY |
| | HHAHDAYLNAVVGTALIKKYPKLESEFVYGDYKVYDVRKMIAKSEQEIGKATAKYFFYSNIMNFFKTEIT |
| | LANGEIRKRPLIETNGETGEIVWDKGRDFATVRKVLSMPQVNIVKKTEVQTGGFSKESILPKRNSDKLIA |
| | RKKDWDPKKYGGFDSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDTTI |
| | DRKRYTSTKEVLDATLIHQSITGLYETRIDLSQLGGD (SEQ ID NO: 113) |

### (v) Other Cas9 Variants

Besides dead Cas9 and Cas9 nickase variants, the Cas9 proteins used herein may also include other “Cas9 variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SEQ ID NO: 74). In exemplary embodiments, any of the base editors of the disclosure may comprise a dead Cas9 having at least 98% or at least 99% identity to the amino acid sequence set forth as SEQ ID NO: 101. In some embodiments, any of the disclosed base editors comprise a dead Cas9 having the sequence of SEQ ID NO: 101.

In some embodiments, the disclosure also may utilize Cas9 fragments which retain their functionality and which are fragments of any herein disclosed Cas9 protein. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In various embodiments, the base editors disclosed herein may comprise one of the Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

In various other embodiments, the napDNAbp can be any of the following proteins: a a Cpf1, a CasX, a CasY, a C2c1, a C2c2, a C2c3, a GeoCas9, a CjCas9, a Cas12a, a Cas12b, a Cas12g, a Cas12h, a Cas12i, a Cas13b, a Cas13c, a Cas13d, a Cas14, a Csn2, an xCas9, an SpCas9-NG, an SpCas9-NG-CP1041, an SpCas9-NG-VRQR, a circularly permuted Cas9, or an Argonaute (Ago) domain, a Cas9-KKH, an SPCas9-VQR, an SpCas9-VRQR, an SpCas9-NRRH, an SpaCas9-NRTH, an SpCas9-NRCH, or a variant thereof.

### (vi) Small-Sized Cas9 Variants

In some embodiments, the base editors contemplated herein can include a Cas9 protein that is of smaller molecular weight than the canonical SpCas9 sequence. In some embodiments, the smaller-sized Cas9 variants may facilitate delivery to cells, e.g., by an expression vector, nanoparticle, or other means of delivery.

The canonical SpCas9 protein is 1368 amino acids in length and has a predicted molecular weight of 158 kilodaltons. The term “small-sized Cas9 variant”, as used herein, refers to any Cas9 variant-naturally occurring, engineered, or otherwise-that is less than at least 1300 amino acids, or at least less than 1290 amino acids, or than less than 1280 amino acids, or less than 1270 amino acid, or less than 1260 amino acid, or less than 1250 amino acids, or less than 1240 amino acids, or less than 1230 amino acids, or less than 1220 amino acids, or less than 1210 amino acids, or less than 1200 amino acids, or less than 1190 amino acids, or less than 1180 amino acids, or less than 1170 amino acids, or less than 1160 amino acids, or less than 1150 amino acids, or less than 1140 amino acids, or less than 1130 amino acids, or less than 1120 amino acids, or less than 1110 amino acids, or less than 1100 amino acids, or less than 1050 amino acids, or less than 1000 amino acids, or less than 950 amino acids, or less than 900 amino acids, or less than 850 amino acids, or less than 800 amino acids, or less than 750 amino acids, or less than 700 amino acids, or less than 650 amino acids, or less than 600 amino acids, or less than 550 amino acids, or less than 500 amino acids, but at least larger than about 400 amino acids and retaining the required functions of the Cas9 protein.

In various embodiments, the base editors disclosed herein may comprise one of the small-sized Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference small-sized Cas9 protein.

| Description | Sequence | SEQ ID NO: |
|---|---|---|
| SaCas9 | MGKRNYILGLDIGITSVGYGIIDYETRDVIDAGVRLFKEANVENNEGRRSKRGARRL | SEQ ID NO: |
| Staphylococcus | KRRRRHRIQRVKKLLFDYNLLTDHSELSGINPYEARVKGLSQKLSEEEFSAALLHLA | 114 |
| aureus | KRRGVHNVNEVEEDTGNELSTKEQISRNSKALEEKYVAELQLERLKKDGEVRGSINR | |
| 1053 AA | FKTSDYVKEAKQLLKVQKAYHQLDQSFIDTYIDLLETRRTYYEGPGEGSPFGWKDIK | |
| 123 kDa | EWYEMLMGHCTYFPEELRSVKYAYNADLYNALNDLNNLVITRDENEKLEYYEKFQII | |
| | ENVFKQKKKPTLKQIAKEILVNEEDIKGYRVTSTGKPEFTNLKVYHDIKDITARKEI | |
| | IENAELLDQIAKILTIYQSSEDIQEELTNLNSELTQEEIEQISNLKGYTGTHNLSLK | |
| | AINLILDELWHTNDNQIAIFNRLKLVPKKVDLSQQKEIPTTLVDDFILSPVVKRSFI | |
| | QSIKVINAIIKKYGLPNDIIIELAREKNSKDAQKMINEMQKRNRQTNERIEEIIRTT | |
| | GKENAKYLIEKIKLHDMQEGKCLYSLEAIPLEDLLNNPFNYEVDHIIPRSVSFDNSF | |
| | NNKVLVKQEENSKKGNRTPFQYLSSSDSKISYETFKKHILNLAKGKGRISKTKKEYL | |
| | LEERDINRFSVQKDFINRNLVDTRYATRGLMNLLRSYFRVNNLDVKVKSINGGFTSF | |
| | LRRKWKFKKERNKGYKHHAEDALIIANADFIFKEWKKLDKAKKVMENQMFEEKQAES | |
| | MPEIETEQEYKEIFITPHQIKHIKDFKDYKYSHRVDKKPNRKLINDTLYSTRKDDKG | |
| | NTLIVNNLNGLYDKDNDKLKKLINKSPEKLLMYHHDPQTYQKLKLIMEQYGDEKNPL | |
| | YKYYEETGNYLTKYSKKDNGPVIKKIKYYGNKLNAHLDITDDYPNSRNKVVKLSLKP | |
| | YRFDVYLDNGVYKFVTVKNLDVIKKENYYEVNSKCYEEAKKLKKISNQAEFIASFYK | |
| | NDLIKINGELYRVIGVNNDLLNRIEVNMIDITYREYLENMNDKRPPHIIKTIASKTQ | |
| | SIKKYSTDILGNLYEVKSKKHPQIIKK | |
| NmeCas 9 | MAAFKPNSINYILGLDIGIASVGWAMVEIDEEENPIRLIDLGVRVFERAEVPKTGDS | SEQ ID NO: |
| N. | LAMARRLARSVRRLTRRRAHRLLRTRRLLKREGVLQAANFDENGLIKSLPNTPWQLR | 115 |
| meningitidis | AAALDRKLTPLEWSAVLLHLIKHRGYLSQRKNEGETADKELGALLKGVAGNAHALQT | |
| 1083 AA | GDFRTPAELALNKFEKESGHIRNQRSDYSHTFSRKDLQAELILLFEKQKEFGNPHVS | |
| 124.5 kDa | GGLKEGIETLLMTQRPALSGDAVQKMLGHCTFEPAEPKAAKNTYTAERFIWLTKLNN | |
| | LRILEQGSERPLTDTERATLMDEPYRKSKLTYAQARKLLGLEDTAFFKGLRYGKDNA | |
| | EASTLMEMKAYHAISRALEKEGLKDKKSPLNLSPELQDEIGTAFSLFKTDEDITGRL | |
| | KDRIQPEILEALLKHISFDKFVQISLKALRRIVPLMEQGKRYDEACAEIYGDHYGKK | |
| | NTEEKIYLPPIPADEIRNPVVLRALSQARKVINGVVRRYGSPARIHIETAREVGKSF | |
| | KDRKEIEKRQEENRKDREKAAAKFREYFPNFVGEPKSKDILKLRLYEQQHGKCLYSG | |
| | KEINLGRLNEKGYVEIDAALPFSRTWDDSFNNKVLVLGSENQNKGNQTPYEYFNGKD | |
| | NSREWQEFKARVETSRFPRSKKQRILLQKFDEDGFKERNLNDTRYVNRFLCQFVADR | |
| | MRLTGKGKKRVFASNGQITNLLRGFWGLRKVRAENDRHHALDAVVVACSTVAMQQKI | |
| | TRFVRYKEMNAFDGKTIDKETGEVLHQKTHFPQPWEFFAQEVMIRVFGKPDGKPEFE | |
| | EADTLEKLRTLLAEKLSSRPEAVHEYVTPLFVSRAPNRKMSGQGHMETVKSAKRLDE | |
| | GVSVLRVPLTQLKLKDLEKMVNREREPKLYEALKARLEAHKDDPAKAFAEPFYKYDK | |
| | AGNRTQQVKAVRVEQVQKTGVWVRNHNGIADNATMVRVDVFEKGDKYYLVPIYSWQV | |
| | AKGILPDRAVVQGKDEEDWQLIDDSFNFKFSLHPNDLVEVITKKARMFGYFASCHRG | |
| | TGNINIRIHDLDHKIGKNGILEGIGVKTALSFQKYQIDELGKEIRPCRLKKRPPVR | |
| CjCas9 | MARILAFDIGISSIGWAFSENDELKDCGVRIFTKVENPKTGESLALPRRLARSARKR | SEQ ID NO: |
| C. jejuni | LARRKARLNHLKHLIANEFKLNYEDYQSFDESLAKAYKGSLISPYELRFRALNELLS | 116 |
| 984 AA | KQDFARVILHIAKRRGYDDIKNSDDKEKGAILKAIKQNEEKLANYQSVGEYLYKEYF | |
| 114.9 kDa | QKFKENSKEFTNVRNKKESYERCIAQSFLKDELKLIFKKQREFGFSFSKKFEEEVLS | |
| | VAFYKRALKDFSHLVGNCSFFTDEKRAPKNSPLAFMFVALTRIINLLNNLKNTEGIL | |
| | YTKDDLNALLNEVLKNGTLTYKQTKKLLGLSDDYEFKGEKGTYFIEFKKYKEFIKAL | |
| | GEHNLSQDDLNEIAKDITLIKDEIKLKKALAKYDLNQNQIDSLSKLEFKDHLNISFK | |
| | ALKLVTPLMLEGKKYDEACNELNLKVAINEDKKDFLPAFNETYYKDEVTNPVVLRAI | |
| | KEYRKVLNALLKKYGKVHKINIELAREVGKNHSQRAKIEKEQNENYKAKKDAELECE | |
| | KLGLKINSKNILKLRLFKEQKEFCAYSGEKIKISDLQDEKMLEIDHIYPYSRSFDDS | |
| | YMNKVLVFTKQNQEKLNQTPFEAFGNDSAKWQKIEVLAKNLPTKKQKRILDKNYKDK | |
| | EQKNFKDRNLNDTRYIARLVLNYTKDYLDFLPLSDDENTKLNDTQKGSKVHVEAKSG | |
| | MLTSALRHTWGFSAKDRNNHLHHAIDAVIIAYANNSIVKAFSDFKKEQESNSAELYA | |
| | KKISELDYKNKRKFFEPFSGFRQKVLDKIDEIFVSKPERKKPSGALHEETFRKEEEF | |
| | YQSYGGKEGVLKALELGKIRKVNGKIVKNGDMFRVDIFKHKKTNKFYAVPIYTMDFA | |
| | LKVLPNKAVARSKKGEIKDWILMDENYEFCFSLYKDSLILIQTKDMQEPEFVYYNAF | |
| | TSSTVSLIVSKHDNKFETLSKNQKILFKNANEKEVIAKSIGIQNLKVFEKYIVSALG | |
| | EVTKAEFRQREDFKK | |
| GeoCas 9 | MRYKIGLDIGITSVGWAVMNLDIPRIEDLGVRIFDRAENPQTGESLALPRRLARSAR | SEQ ID NO: |
| G. | RRLRRRKHRLERIRRLVIREGILTKEELDKLFEEKHEIDVWQLRVEALDRKLNNDEL | 117 |
| stearothermo | ARVLLHLAKRRGFKSNRKSERSNKENSTMLKHIEENRAILSSYRTVGEMIVKDPKFA | |
| philus | LHKRNKGENYTNTIARDDLEREIRLIFSKQREFGNMSCTEEFENEYITIWASQRPVA | |
| 1087 AA | SKDDIEKKVGFCTFEPKEKRAPKATYTFQSFIAWEHINKLRLISPSGARGLTDEERR | |
| 127 kDa | LLYEQAFQKNKITYHDIRTLLHLPDDTYFKGIVYDRGESRKQNENIRFLELDAYHQI | |
| | RKAVDKVYGKGKSSSFLPIDFDTFGYALTLFKDDADIHSYLRNEYEQNGKRMPNLAN | |
| | KVYDNELIEELLNLSFTKFGHLSLKALRSILPYMEQGEVYSSACERAGYTFTGPKKK | |
| | QKTMLLPNIPPIANPVVMRALTQARKVVNAIIKKYGSPVSIHIELARDLSQTFDERR | |
| | KTKKEQDENRKKNETAIRQLMEYGLTLNPTGHDIVKFKLWSEQNGRCAYSLQPIEIE | |
| | RLLEPGYVEVDHVIPYSRSLDDSYTNKVLVLTRENREKGNRIPAEYLGVGTERWQQF | |
| | ETFVLTNKQFSKKKRDRLLRLHYDENEETEFKNRNLNDTRYISRFFANFIREHLKFA | |
| | ESDDKQKVYTVNGRVTAHLRSRWEFNKNREESDLHHAVDAVIVACTTPSDIAKVTAF | |
| | YQRREQNKELAKKTEPHFPQPWPHFADELRARLSKHPKESIKALNLGNYDDQKLESL | |
| | QPVFVSRMPKRSVTGAAHQETLRRYVGIDERSGKIQTVVKTKLSEIKLDASGHFPMY | |
| | GKESDPRTYEAIRQRLLEHNNDPKKAFQEPLYKPKKNGEPGPVIRTVKIIDTKNQVI | |
| | PLNDGKTVAYNSNIVRVDVFEKDGKYYCVPVYTMDIMKGILPNKAIEPNKPYSEWKE | |
| | MTEDYTFRFSLYPNDLIRIELPREKTVKTAAGEEINVKDVFVYYKTIDSANGGLELI | |
| | SHDHRFSLRGVGSRTLKRFEKYQVDVLGNIYKVRGEKRVGLASSAHSKPGKTIRPLQ | |
| | STRD | |
| LbaCas12a | MSKLEKFTNCYSLSKTLRFKAIPVGKTQENIDNKRLLVEDEKRAEDYKGVKKLLDRY | SEQ ID NO: |
| L. bacterium | YLSFINDVLHSIKLKNLNNYISLFRKKTRTEKENKELENLEINLRKEIAKAFKGNEG | 118 |
| 1228 AA | YKSLFKKDIIETILPEFLDDKDEIALVNSFNGFTTAFTGFFDNRENMFSEEAKSTSI | |
| 143.9 kDa | AFRCINENLTRYISNMDIFEKVDAIFDKHEVQEIKEKILNSDYDVEDFFEGEFFNFV | |
| | LTQEGIDVYNAIIGGFVTESGEKIKGLNEYINLYNQKTKQKLPKFKPLYKQVLSDRE | |
| | SLSFYGEGYTSDEEVLEVFRNTLNKNSEIFSSIKKLEKLFKNFDEYSSAGIFVKNGP | |
| | AISTISKDIFGEWNVIRDKWNAEYDDIHLKKKAVVTEKYEDDRRKSFKKIGSFSLEQ | |
| | LQEYADADLSVVEKLKEIIIQKVDEIYKVYGSSEKLFDADFVLEKSLKKNDAVVAIM | |
| | KDLLDSVKSFENYIKAFFGEGKETNRDESFYGDFVLAYDILLKVDHIYDAIRNYVTQ | |
| | KPYSKDKFKLYFQNPQFMGGWDKDKETDYRATILRYGSKYYLAIMDKKYAKCLQKID | |
| | KDDVNGNYEKINYKLLPGPNKMLPKVFFSKKWMAYYNPSEDIQKIYKNGTFKKGDMF | |
| | NLNDCHKLIDFFKDSISRYPKWSNAYDFNFSETEKYKDIAGFYREVEEQGYKVSFES | |
| | ASKKEVDKLVEEGKLYMFQIYNKDFSDKSHGTPNLHTMYFKLLFDENNHGQIRLSGG | |
| | AELFMRRASLKKEELVVHPANSPIANKNPDNPKKTTTLSYDVYKDKRFSEDQYELHI | |
| | PIAINKCPKNIFKINTEVRVLLKHDDNPYVIGIDRGERNLLYIVVVDGKGNIVEQYS | |
| | LNEIINNFNGIRIKTDYHSLLDKKEKERFEARQNWTSIENIKELKAGYISQVVHKIC | |
| | ELVEKYDAVIALEDLNSGFKNSRVKVEKQVYQKFEKMLIDKLNYMVDKKSNPCATGG | |
| | ALKGYQITNKFESFKSMSTQNGFIFYIPAWLTSKIDPSTGFVNLLKTKYTSIADSKK | |
| | FISSFDRIMYVPEEDLFEFALDYKNFSRTDADYIKKWKLYSYGNRIRIFRNPKKNNV | |
| | FDWEEVCLTSAYKELFNKYGINYQQGDIRALLCEQSDKAFYSSFMALMSLMLQMRNS | |
| | ITGRTDVDFLISPVKNSDGIFYDSRNYEAQENAILPKNADANGAYNIARKVLWAIGQ | |
| | FKKAEDEKLDKVKIAISNKEWLEYAQTSVKH | |
| BhCas12b | MATRSFILKIEPNEEVKKGLWKTHEVLNHGIAYYMNILKLIRQEAIYEHHEQDPKNP | SEQ ID NO: |
| B. hisashii | KKVSKAEIQAELWDFVLKMQKCNSFTHEVDKDEVFNILRELYEELVPSSVEKKGEAN | 119 |
| 1108 AA | QLSNKFLYPLVDPNSQSGKGTASSGRKPRWYNLKIAGDPSWEEEKKKWEEDKKKDPL | |
| 130.4 kDa | AKILGKLAEYGLIPLFIPYTDSNEPIVKEIKWMEKSRNQSVRRLDKDMFIQALERFL | |
| | SWESWNLKVKEEYEKVEKEYKTLEERIKEDIQALKALEQYEKERQEQLLRDTLNTNE | |
| | YRLSKRGLRGWREIIQKWLKMDENEPSEKYLEVFKDYQRKHPREAGDYSVYEFLSKK | |
| | ENHFIWRNHPEYPYLYATFCEIDKKKKDAKQQATFTLADPINHPLWVRFEERSGSNL | |
| | NKYRILTEQLHTEKLKKKLTVQLDRLIYPTESGGWEEKGKVDIVLLPSRQFYNQIFL | |
| | DIEEKGKHAFTYKDESIKFPLKGTLGGARVQFDRDHLRRYPHKVESGNVGRIYFNMT | |
| | VNIEPTESPVSKSLKIHRDDFPKVVNFKPKELTEWIKDSKGKKLKSGIESLEIGLRV | |
| | MSIDLGQRQAAAASIFEVVDQKPDIEGKLFFPIKGTELYAVHRASFNIKLPGETLVK | |
| | SREVLRKAREDNLKLMNQKLNFLRNVLHFQQFEDITEREKRVTKWISRQENSDVPLV | |
| | YQDELIQIRELMYKPYKDWVAFLKQLHKRLEVEIGKEVKHWRKSLSDGRKGLYGISL | |
| | KNIDEIDRTRKFLLRWSLRPTEPGEVRRLEPGQRFAIDQLNHLNALKEDRLKKMANT | |
| | IIMHALGYCYDVRKKKWQAKNPACQIILFEDLSNYNPYEERSRFENSKLMKWSRREI | |
| | PRQVALQGEIYGLQVGEVGAQFSSRFHAKTGSPGIRCSVVTKEKLQDNRFFKNLQRE | |
| | GRLTLDKIAVLKEGDLYPDKGGEKFISLSKDRKCVTTHADINAAQNLQKRFWTRTHG | |
| | FYKVYCKAYQVDGQTVYIPESKDQKQKIIEEFGEGYFILKDGVYEWVNAGKLKIKKG | |
| | SSKQSSSELVDSDILKDSFDLASELKGEKLMLYRDPSGNVFPSDKWMAAGVFFGKLE | |
| | RILISKLTNQYSISTIEDDSSKQSM | |

(vii) Other Cas9 Equivalents

In some embodiments, the base editors described herein can include any Cas9 equivalent. As used herein, the term “Cas9 equivalent” is a broad term that encompasses any napDNAbp protein that serves the same function as Cas9 in the present base editors despite that its amino acid primary sequence and/or its three-dimensional structure may be different and/or unrelated from an evolutionary standpoint. Thus, while Cas9 equivalents include any Cas9 ortholog, homolog, mutant, or variant described or embraced herein that are evolutionarily related, the Cas9 equivalents also embrace proteins that may have evolved through convergent evolution processes to have the same or similar function as Cas9, but which do not necessarily have any similarity with regard to amino acid sequence and/or three dimensional structure. The base editors described here embrace any Cas9 equivalent that would provide the same or similar function as Cas9 despite that the Cas9 equivalent may be based on a protein that arose through convergent evolution.

For example, CasX is a Cas9 equivalent that reportedly has the same function as Cas9 but which evolved through convergent evolution. Thus, the CasX protein described in Liu et al., “CasX enzymes comprises a distinct family of RNA-guided genome editors,” Nature, 2019, Vol.566: 218-223, is contemplated to be used with the base editors described herein. In addition, any variant or modification of CasX is conceivable and within the scope of the present disclosure.

Cas9 is a bacterial enzyme that evolved in a wide variety of species. However, the Cas9 equivalents contemplated herein may also be obtained from archaea, which constitute a domain and kingdom of single-celled prokaryotic microbes different from bacteria.

In some embodiments, Cas9 equivalents may refer to CasX or CasY, which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a nucleic acid programmable DNA binding protein (napDNAbp), and are within the scope of this disclosure. Also see Liu et al., “CasX enzymes comprises a distinct family of RNA-guided genome editors,” Nature, 2019, Vol.566: 218-223. Any of these Cas9 equivalents are contemplated.

In some embodiments, the Cas9 equivalent comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring CasX or CasY protein. In some embodiments, the napDNAbp is a naturally-occurring CasX or CasY protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a wild-type Cas moiety or any Cas moiety provided herein.

In various embodiments, the nucleic acid programmable DNA binding proteins include, without limitation, Cas9 (e.g., dCas9 and nCas9), CasX, CasY, Cpf1, C2c1, C2c2, C2C3, Argonaute, Cas12a, and Cas12b. Examples of Cas12b napDNAbp domains include Bacillus hisashii Cas12b (BhCas12b), Alicyclobacillus acidiphilus Cas12b (AaCas12b), and Bacillus sp. V3-13 (BvCas12b). One example of a nucleic acid programmable DNA-binding protein that has different PAM specificity than Cas9 is Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference. The state of the art may also now refer to Cpf1 enzymes as Cas12a.

In still other embodiments, the Cas protein may include any CRISPR associated protein, including but not limited to, Cas12a, Cas12b, Casl, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2. Csm2, Csm3, Csm4, Csm5, Csm6, Cmrl, Cmr3, Cmr4, Cmr5, Cmr6, Csb1, Csb2, Csb3, Csx17, Csx14, Csx10, Csx16, CsaX, Csx3, Csx1, Csx15, Csfl, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO:74).

In various other embodiments, the napDNAbp can be any of the following proteins: a Cas9, a Cpf1, a CasX, a CasY, a C2c1, a C2c2, a C2c3, a GeoCas9, a CjCas9, a Cas12a, a Cas12b, a Cas12g, a Cas12h, a Cas12i, a Cas13b, a Cas13c, a Cas13d, a Cas14, a Csn2, an xCas9, an SpCas9-NG, a circularly permuted Cas9 domain such as CP1012, CP1028, CP1041, CP1249, and CP1300, or an Argonaute (Ago) domain, a Cas9-KKH, a SmacCas9, a Spy-macCas9, an SpCas9-VRQR, an SpCas9-NG-VRQR, an SpCas9-VRER, an SpCas9-VQR, an SpCas9-NG-VQR, an SpCas9-EQR, an SpCas9-NRRH, an SpaCas9-NRTH, an SpCas9-NRCH, an LbCas12a, an AsCas12a, a CeCas12a, an MbCas12a, Cas(I), or a variant thereof.

Exemplary Cas9 equivalent protein sequences can include the following:

| Description | Sequence |
|---|---|
| AsCas12a | MTQFEGFTNLYQVSKTLRFELIPQGKTLKHIQEQGFIEEDKARNDHYKELKPIIDRIYKTYADQCLQLVQL |
| (previously | DWENLSAAIDSYRKEKTEETRNALIEEQATYRNAIHDYFIGRTDNLTDAINKRHAEIYKGLFKAELFNGKV |
| known as | LKQLGTVTTTEHENALLRSFDKFTTYFSGFYENRKNVFSAEDISTAIPHRIVQDNFPKFKENCHIFTRLIT |
| Cpf1) | AVPSLREHFENVKKAIGIFVSTSIEEVFSFPFYNQLLTQTQIDLYNQLLGGISREAGTEKIKGLNEVLNLA |
| Acidaminocoecus | IQKNDETAHIIASLPHRFIPLFKQILSDRNTLSFILEEFKSDEEVIQSFCKYKTLLRNENVLETAEALFNE |
| sp. | LNSIDLTHIFISHKKLETISSALCDHWDTLRNALYERRISELTGKITKSAKEKVRQRSLKHEDINLQEIIS |
| (strain | AAGKELSEAFKQKTSEILSHAHAALDQPLPTTLKKQEEKEILKSQLDSLLGLYHLLDWFAVDESNEVDPEF |
| BV3L6) | SARLTGIKLEMEPSLSFYNKARNYATKKPYSVEKFKLNFQMPTLASGWDVNKEKNNGAILFVKNGLYYLGI |
| UniProtKB | MPKQKGRYKALSFEPTEKTSEGFDKMYYDYFPDAAKMIPKCSTQLKAVTAHFQTHTTPILLSNNFIEPLEI |
| U2UMQ6 | TKEIYDLNNPEKEPKKFQTAYAKKTGDQKGYREALCKWIDFTRDFLSKYTKTTSIDLSSLRPSSQYKDLGE |
| | YYAELNPLLYHISFQRIAEKEIMDAVETGKLYLFQIYNKDFAKGHHGKPNLHTLYWTGLFSPENLAKTSIK |
| | LNGQAELFYRPKSRMKRMAHRLGEKMLNKKLKDQKTPIPDTLYQELYDYVNHRLSHDLSDEARALLPNVIT |
| | KEVSHEIIKDRRFTSDKFFFHVPITLNYQAANSPSKFNQRVNAYLKEHPETPIIGIDRGERNLIYITVIDS |
| | TGKILEQRSLNTIQQFDYQKKLDNREKERVAARQAWSVVGTIKDLKQGYLSQVIHEIVDLMIHYQAVVVLE |
| | NLNFGFKSKRTGIAEKAVYQQFEKMLIDKLNCLVLKDYPAEKVGGVLNPYQLTDQFTSFAKMGTQSGFLFY |
| | VPAPYTSKIDPLTGFVDPFVWKTIKNHESRKHFLEGFDFLHYDVKTGDFILHFKMNRNLSFQRGLPGFMPA |
| | WDIVFEKNETQFDAKGTPFIAGKRIVPVIENHRFTGRYRDLYPANELIALLEEKGIVFRDGSNILPKLLEN |
| | DDSHAIDTMVALIRSVLQMRNSNAATGEDYINSPVRDLNGVCFDSRFQNPEWPMDADANGAYHIALKGQLL |
| | LNHLKESKDLKLQNGISNQDWLAYIQELRN (SEQ ID NO: 120) |
| AsCas12a | MTQFEGFTNLYQVSKTLRFELIPQGKTLKHIQEQGFIEEDKARNDHYKELKPIIDRIYKTYADQCLQLVQL |
| nickase | DWENLSAAIDSYRKEKTEETRNALIEEQATYRNAIHDYFIGRTDNLTDAINKRHAEIYKGLFKAELFNGKV |
| (e.g., | LKQLGTVTTTEHENALLRSFDKFTTYFSGFYENRKNVFSAEDISTAIPHRIVQDNFPKFKENCHIFTRLIT |
| R1226A) | AVPSLREHFENVKKAIGIFVSTSIEEVFSFPFYNQLLTQTQIDLYNQLLGGISREAGTEKIKGLNEVLNLA |
| | IQKNDETAHIIASLPHRFIPLFKQILSDRNTLSFILEEFKSDEEVIQSFCKYKTLLRNENVLETAEALFNE |
| | LNSIDLTHIFISHKKLETISSALCDHWDTLRNALYERRISELTGKITKSAKEKVRQRSLKHEDINLQEIIS |
| | AAGKELSEAFKQKTSEILSHAHAALDQPLPTTLKKQEEKEILKSQLDSLLGLYHLLDWFAVDESNEVDPEF |
| | SARLTGIKLEMEPSLSFYNKARNYATKKPYSVEKFKLNFQMPTLASGWDVNKEKNNGAILFVKNGLYYLGI |
| | MPKQKGRYKALSFEPTEKTSEGFDKMYYDYFPDAAKMIPKCSTQLKAVTAHFQTHTTPILLSNNFIEPLEI |
| | TKEIYDLNNPEKEPKKFQTAYAKKTGDQKGYREALCKWIDFTRDFLSKYTKTTSIDLSSLRPSSQYKDLGE |
| | YYAELNPLLYHISFQRIAEKEIMDAVETGKLYLFQIYNKDFAKGHHGKPNLHTLYWTGLFSPENLAKTSIK |
| | LNGQAELFYRPKSRMKRMAHRLGEKMLNKKLKDQKTPIPDTLYQELYDYVNHRLSHDLSDEARALLPNVIT |
| | KEVSHEIIKDRRFTSDKFFFHVPITLNYQAANSPSKFNQRVNAYLKEHPETPIIGIDRGERNLIYITVIDS |
| | TGKILEQRSLNTIQQFDYQKKLDNREKERVAARQAWSVVGTIKDLKQGYLSQVIHEIVDLMIHYQAVVVLE |
| | NLNFGFKSKRTGIAEKAVYQQFEKMLIDKLNCLVLKDYPAEKVGGVLNPYQLTDQFTSFAKMGTQSGFLFY |
| | VPAPYTSKIDPLTGFVDPFVWKTIKNHESRKHFLEGFDFLHYDVKTGDFILHFKMNRNLSFQRGLPGFMPA |
| | WDIVFEKNETQFDAKGTPFIAGKRIVPVIENHRFTGRYRDLYPANELIALLEEKGIVFRDGSNILPKLLEN |
| | DDSHAIDTMVALIRSVLQMANSNAATGEDYINSPVRDLNGVCFDSRFQNPEWPMDADANGAYHIALKGQLL |
| | LNHLKESKDLKLQNGISNQDWLAYIQELRN (SEQ ID NO: 121) |
| LbCas12a | MNYKTGLEDFIGKESLSKTLRNALIPTESTKIHMEEMGVIRDDELRAEKQQELKEIMDDYYRTFIEEKLGQ |
| (previously | IQGIQWNSLFQKMEETMEDISVRKDLDKIQNEKRKEICCYFTSDKRFKDLFNAKLITDILPNFIKDNKEYT |
| known as | EEEKAEKEQTRVLFQRFATAFTNYFNQRRNNFSEDNISTAISFRIVNENSEIHLQNMRAFQRIEQQYPEEV |
| Cpf1) | CGMEEEYKDMLQEWQMKHIYSVDFYDRELTQPGIEYYNGICGKINEHMNQFCQKNRINKNDFRMKKLHKQI |
| Lachnospiraceae | LCKKSSYYEIPFRFESDQEVYDALNEFIKTMKKKEIIRRCVHLGQECDDYDLGKIYISSNKYEQISNALYG |
| bacterium | SWDTIRKCIKEEYMDALPGKGEKKEEKAEAAAKKEEYRSIADIDKIISLYGSEMDRTISAKKCITEICDMA |
| GAM 7 9 | GQISIDPLVCNSDIKLLQNKEKTTEIKTILDSFLHVYQWGQTFIVSDIIEKDSYFYSELEDVLEDFEGITT |
| Ref Seq. | LYNHVRSYVTQKPYSTVKFKLHFGSPTLANGWSQSKEYDNNAILLMRDQKFYLGIFNVRNKPDKQIIKGHE |
| WP_11962338 | KEEKGDYKKMIYNLLPGPSKMLPKVFITSRSGQETYKPSKHILDGYNEKRHIKSSPKFDLGYCWDLIDYYK |
| 2.1 | ECIHKHPDWKNYDFHFSDTKDYEDISGFYREVEMQGYQIKWTYISADEIQKLDEKGQIFLFQIYNKDFSVH |
| | STGKDNLHTMYLKNLFSEENLKDIVLKLNGEAELFFRKASIKTPIVHKKGSVLVNRSYTQTVGNKEIRVSI |
| | PEEYYTEIYNYLNHIGKGKLSSEAQRYLDEGKIKSFTATKDIVKNYRYCCDHYFLHLPITINFKAKSDVAV |
| | NERTLAYIAKKEDIHIIGIDRGERNLLYISVVDVHGNIREQRSFNIVNGYDYQQKLKDREKSRDAARKNWE |
| | EIEKIKELKEGYLSMVIHYIAQLVVKYNAVVAMEDLNYGFKTGRFKVERQVYQKFETMLIEKLHYLVFKDR |
| | EVCEEGGVLRGYQLTYIPESLKKVGKQCGFIFYVPAGYTSKIDPTTGFVNLFSFKNLTNRESRQDFVGKFD |
| | EIRYDRDKKMFEFSFDYNNYIKKGTILASTKWKVYTNGTRLKRIVVNGKYTSQSMEVELTDAMEKMLQRAG |
| | IEYHDGKDLKGQIVEKGIEAEIIDIFRLTVQMRNSRSESEDREYDRLISPVLNDKGEFFDTATADKTLPQD |
| | ADANGAYCIALKGLYEVKQIKENWKENEQFPRNKLVQDNKTWFDFMQKKRYL (SEQ ID NO: 122) |
| PcCas12a- | MAKNFEDFKRLYSLSKTLRFEAKPIGATLDNIVKSGLLDEDEHRAASYVKVKKLIDEYHKVFIDRVLDDGC |
| previously | LPLENKGNNNSLAEYYESYVSRAQDEDAKKKFKEIQQNLRSVIAKKLTEDKAYANLFGNKLIESYKDKEDK |
| known at | KKIIDSDLIQFINTAESTQLDSMSQDEAKELVKEFWGFVTYFYGFFDNRKNMYTAEEKSTGIAYRLVNENL |
| Cpf1 | PKFIDNIEAFNRAITRPEIQENMGVLYSDFSEYLNVESIQEMFQLDYYNMLLTQKQIDVYNAIIGGKIDDE |
| Prevotella | HDVKIKGINEYINLYNQQHKDDKLPKLKALFKQILSDRNAISWLPEEFNSDQEVLNAIKDCYERLAENVLG |
| copri | DKVLKSLLGSLADYSLDGIFIRNDLQLTDISQKMFGNWGVIQNAIMQNIKRVAPARKHKESEEDYEKRIAG |
| Ref Seq. | IFKKADSFSISYINDCLNEADPNNAYFVENYFATFGAVNTPTMQRENLFALVQNAYTEVAALLHSDYPTVK |
| WP_11922772 | HLAQDKANVSKIKALLDAIKSLQHFVKPLLGKGDESDKDERFYGELASLWAELDTVTPLYNMIRNYMTRKP |
| 6.1 | YSQKKIKLNFENPQLLGGWDANKEKDYATIILRRNGLYYLAIMDKDSRKLLGKAMPSDGECYEKMVYKFFK |
| | DVTTMIPKCSTQLKDVQAYFKVNTDDYVLNSKAFNKPLTITKEVFDLNNVLYGKYKKFQKGYLTATGDNVG |
| | YTHAVNVWIKFCMDFLNSYDSTCIYDFSSLKPESYLSLDAFYQDANLLLYKLSFARASVSYINQLVEEGKM |
| | YLFQIYNKDFSEYSKGTPNMHTLYWKALFDERNLADVVYKLNGQAEMFYRKKSIENTHPTHPANHPILNKN |
| | KDNKKKESLFDYDLIKDRRYTVDKFMFHVPITMNFKSVGSENINQDVKAYLRHADDMHIIGIDRGERHLLY |
| | LVVIDLQGNIKEQYSLNEIVNEYNGNTYHTNYHDLLDVREEERLKARQSWQTIENIKELKEGYLSQVIHKI |
| | TQLMVRYHAIVVLEDLSKGFMRSRQKVEKQVYQKFEKMLIDKLNYLVDKKTDVSTPGGLLNAYQLTCKSDS |
| | SQKLGKQSGFLFYIPAWNTSKIDPVTGFVNLLDTHSLNSKEKIKAFFSKFDAIRYNKDKKWFEFNLDYDKF |
| | GKKAEDTRTKWTLCTRGMRIDTFRNKEKNSQWDNQEVDLTTEMKSLLEHYYIDIHGNLKDAISAQTDKAFF |
| | TGLLHILKLTLQMRNSITGTETDYLVSPVADENGIFYDSRSCGNQLPENADANGAYNIARKGLMLIEQIKN |
| | AEDLNNVKFDISNKAWLNFAQQKPYKNG (SEQ ID NO: 123) |
| ErCas12a- | MFSAKLISDILPEFVIHNNNYSASEKEEKTQVIKLFSRFATSFKDYFKNRANCFSANDISSSSCHRIVNDN |
| previously | AEIFFSNALVYRRIVKNLSNDDINKISGDMKDSLKEMSLEEIYSYEKYGEFITQEGISFYNDICGKVNLFM |
| known at | NLYCQKNKENKNLYKLRKLHKQILCIADTSYEVPYKFESDEEVYQSVNGFLDNISSKHIVERLRKIGENYN |
| Cpf1 | GYNLDKIYIVSKFYESVSQKTYRDWETINTALEIHYNNILPGNGKSKADKVKKAVKNDLQKSITEINELVS |
| Eubacterium | NYKLCPDDNIKAETYIHEISHILNNFEAQELKYNPEIHLVESELKASELKNVLDVIMNAFHWCSVFMTEEL |
| rectale | VDKDNNFYAELEEIYDEIYPVISLYNLVRNYVTQKPYSTKKIKLNFGIPTLADGWSKSKEYSNNAIILMRD |
| Ref Seq. | NLYYLGIFNAKNKPDKKIIEGNTSENKGDYKKMIYNLLPGPNKMIPKVFLSSKTGVETYKPSAYILEGYKQ |
| WP_11922364 | NKHLKSSKDFDITFCHDLIDYFKNCIAIHPEWKNFGFDFSDTSTYEDISGFYREVELQGYKIDWTYISEKD |
| 2.1 | IDLLQEKGQLYLFQIYNKDFSKKSSGNDNLHTMYLKNLFSEENLKDIVLKLNGEAEIFFRKSSIKNPIIHK |
| | KGSILVNRTYEAEEKDQFGNIQIVRKTIPENIYQELYKYFNDKSDKELSDEAAKLKNVVGHHEAATNIVKD |
| | YRYTYDKYFLHMPITINFKANKTSFINDRILQYIAKEKDLHVIGIDRGERNLIYVSVIDTCGNIVEQKSFN |
| | IVNGYDYQIKLKQQEGARQIARKEWKEIGKIKEIKEGYLSLVIHEISKMVIKYNAIIAMEDLSYGFKKGRF |
| | KVERQVYQKFETMLINKLNYLVFKDISITENGGLLKGYQLTYIPDKLKNVGHQCGCIFYVPAAYTSKIDPT |
| | TGFVNIFKFKDLTVDAKREFIKKFDSIRYDSDKNLFCFTFDYNNFITQNTVMSKSSWSVYTYGVRIKRRFV |
| | NGRFSNESDTIDITKDMEKTLEMTDINWRDGHDLRQDIIDYEIVQHIFEIFKLTVQMRNSLSELEDRDYDR |
| | LISPVLNENNIFYDSAKAGDALPKDADANGAYCIALKGLYEIKQITENWKEDGKFSRDKLKISNKDWFDFI |
| | QNKRYL (SEQ ID NO: 124) |
| CsCas12a- | MNYKTGLEDFIGKESLSKTLRNALIPTESTKIHMEEMGVIRDDELRAEKQQELKEIMDDYYRAFIEEKLGQ |
| previously | IQGIQWNSLFQKMEETMEDISVRKDLDKIQNEKRKEICCYFTSDKRFKDLFNAKLITDILPNFIKDNKEYT |
| known at | EEEKAEKEQTRVLFQRFATAFTNYFNQRRNNFSEDNISTAISFRIVNENSEIHLQNMRAFQRIEQQYPEEV |
| Cpf1 | CGMEEEYKDMLQEWQMKHIYLVDFYDRVLTQPGIEYYNGICGKINEHMNQFCQKNRINKNDFRMKKLHKQI |
| Clostridium | LCKKSSYYEIPFRFESDQEVYDALNEFIKTMKEKEIICRCVHLGQKCDDYDLGKIYISSNKYEQISNALYG |
| sp. AF34- | SWDTIRKCIKEEYMDALPGKGEKKEEKAEAAAKKEEYRSIADIDKIISLYGSEMDRTISAKKCITEICDMA |
| 10BH | GQISTDPLVCNSDIKLLQNKEKTTEIKTILDSFLHVYQWGQTFIVSDIIEKDSYFYSELEDVLEDFEGITT |
| Ref Seq. | LYNHVRSYVTQKPYSTVKFKLHFGSPTLANGWSQSKEYDNNAILLMRDQKFYLGIFNVRNKPDKQIIKGHE |
| WP_11853841 | KEEKGDYKKMIYNLLPGPSKMLPKVFITSRSGQETYKPSKHILDGYNEKRHIKSSPKFDLGYCWDLIDYYK |
| 8.1 | ECIHKHPDWKNYDFHFSDTKDYEDISGFYREVEMQGYQIKWTYISADEIQKLDEKGQIFLFQIYNKDFSVH |
| | STGKDNLHTMYLKNLFSEENLKDIVLKLNGEAELFFRKASIKTPVVHKKGSVLVNRSYTQTVGDKEIRVSI |
| | PEEYYTEIYNYLNHIGRGKLSTEAQRYLEERKIKSFTATKDIVKNYRYCCDHYFLHLPITINFKAKSDIAV |
| | NERTLAYIAKKEDIHIIGIDRGERNLLYISVVDVHGNIREQRSFNIVNGYDYQQKLKDREKSRDAARKNWE |
| | EIEKIKELKEGYLSMVIHYIAQLVVKYNAVVAMEDLNYGFKTGRFKVERQVYQKFETMLIEKLHYLVFKDR |
| | EVCEEGGVLRGYQLTYIPESLKKVGKQCGFIFYVPAGYTSKIDPTTGFVNLFSFKNLTNRESRQDFVGKFD |
| | EIRYDRDKKMFEFSFDYNNYIKKGTMLASTKWKVYTNGTRLKRIVVNGKYTSQSMEVELTDAMEKMLQRAG |
| | IEYHDGKDLKGQIVEKGIEAEIIDIFRLTVQMRNSRSESEDREYDRLISPVLNDKGEFFDTATADKTLPQD |
| | ADANGAYCIALKGLYEVKQIKENWKENEQFPRNKLVQDNKTWFDFMQKKRYL (SEQ ID NO: 125) |
| BhCas12b | MATRSFILKIEPNEEVKKGLWKTHEVLNHGIAYYMNILKLIRQEAIYEHHEQDPKNPKKVSKAEIQAELWD |
| Bacillus | FVLKMQKCNSFTHEVDKDEVFNILRELYEELVPSSVEKKGEANQLSNKFLYPLVDPNSQSGKGTASSGRKP |
| hisashii | RWYNLKIAGDPSWEEEKKKWEEDKKKDPLAKILGKLAEYGLIPLFIPYTDSNEPIVKEIKWMEKSRNQSVR |
| Ref Seq. | RLDKDMFIQALERFLSWESWNLKVKEEYEKVEKEYKTLEERIKEDIQALKALEQYEKERQEQLLRDTLNTN |
| WP_09514251 | EYRLSKRGLRGWREIIQKWLKMDENEPSEKYLEVFKDYQRKHPREAGDYSVYEFLSKKENHFIWRNHPEYP |
| 5.1 | YLYATFCEIDKKKKDAKQQATFTLADPINHPLWVRFEERSGSNLNKYRILTEQLHTEKLKKKLTVQLDRLI |
| | YPTESGGWEEKGKVDIVLLPSRQFYNQIFLDIEEKGKHAFTYKDESIKFPLKGTLGGARVQFDRDHLRRYP |
| | HKVESGNVGRIYFNMTVNIEPTESPVSKSLKIHRDDFPKVVNFKPKELTEWIKDSKGKKLKSGIESLEIGL |
| | RVMSIDLGQRQAAAASIFEVVDQKPDIEGKLFFPIKGTELYAVHRASFNIKLPGETLVKSREVLRKAREDN |
| | LKLMNQKLNFLRNVLHFQQFEDITEREKRVTKWISRQENSDVPLVYQDELIQIRELMYKPYKDWVAFLKQL |
| | HKRLEVEIGKEVKHWRKSLSDGRKGLYGISLKNIDEIDRTRKFLLRWSLRPTEPGEVRRLEPGQRFAIDQL |
| | NHLNALKEDRLKKMANTIIMHALGYCYDVRKKKWQAKNPACQIILFEDLSNYNPYEERSRFENSKLMKWSR |
| | REIPRQVALQGEIYGLQVGEVGAQFSSRFHAKTGSPGIRCSVVTKEKLQDNRFFKNLQREGRLTLDKIAVL |
| | KEGDLYPDKGGEKFISLSKDRKCVTTHADINAAQNLQKRFWTRTHGFYKVYCKAYQVDGQTVYIPESKDQK |
| | QKIIEEFGEGYFILKDGVYEWVNAGKLKIKKGSSKQSSSELVDSDILKDSFDLASELKGEKLMLYRDPSGN |
| | VFPSDKWMAAGVFFGKLERILISKLTNQYSISTIEDDSSKQSM (SEQ ID NO: 126) |
| ThCas12b | MSEKTTQRAYTLRLNRASGECAVCQNNSCDCWHDALWATHKAVNRGAKAFGDWLLTLRGGLCHTLVEMEVP |
| Thermomonas | AKGNNPPQRPTDQERRDRRVLLALSWLSVEDEHGAPKEFIVATGRDSADDRAKKVEEKLREILEKRDFQEH |
| hydrothermalis | EIDAWLQDCGPSLKAHIREDAVWVNRRALFDAAVERIKTLTWEEAWDFLEPFFGTQYFAGIGDGKDKDDAE |
| Ref Seq. | GPARQGEKAKDLVQKAGQWLSARFGIGTGADFMSMAEAYEKIAKWASQAQNGDNGKATIEKLACALRPSEP |
| WP_07275483 | PTLDTVLKCISGPGHKSATREYLKTLDKKSTVTQEDLNQLRKLADEDARNCRKKVGKKGKKPWADEVLKDV |
| 8 | ENSCELTYLQDNSPARHREFSVMLDHAARRVSMAHSWIKKAEQRRRQFESDAQKLKNLQERAPSAVEWLDR |
| | FCESRSMTTGANTGSGYRIRKRAIEGWSYVVQAWAEASCDTEDKRIAAARKVQADPEIEKFGDIQLFEALA |
| | ADEAICVWRDQEGTQNPSILIDYVTGKTAEHNQKRFKVPAYRHPDELRHPVFCDFGNSRWSIQFAIHKEIR |
| | DRDKGAKQDTRQLQNRHGLKMRLWNGRSMTDVNLHWSSKRLTADLALDQNPNPNPTEVTRADRLGRAASSA |
| | FDHVKIKNVFNEKEWNGRLQAPRAELDRIAKLEEQGKTEQAEKLRKRLRWYVSFSPCLSPSGPFIVYAGQH |
| | NIQPKRSGQYAPHAQANKGRARLAQLILSRLPDLRILSVDLGHRFAAACAVWETLSSDAFRREIQGLNVLA |
| | GGSGEGDLFLHVEMTGDDGKRRTVVYRRIGPDQLLDNTPHPAPWARLDRQFLIKLQGEDEGVREASNEELW |
| | TVHKLEVEVGRTVPLIDRMVRSGFGKTEKQKERLKKLRELGWISAMPNEPSAETDEKEGEIRSISRSVDEL |
| | MSSALGTLRLALKRHGNRARIAFAMTADYKPMPGGQKYYFHEAKEASKNDDETKRRDNQIEFLQDALSLWH |
| | DLFSSPDWEDNEAKKLWQNHIATLPNYQTPEEISAELKRVERNKKRKENRDKLRTAAKALAENDQLRQHLH |
| | DTWKERWESDDQQWKERLRSLKDWIFPRGKAEDNPSIRHVGGLSITRINTISGLYQILKAFKMRPEPDDLR |
| | KNIPQKGDDELENFNRRLLEARDRLREQRVKQLASRIIEAALGVGRIKIPKNGKLPKRPRTTVDTPCHAVV |
| | IESLKTYRPDDLRTRRENRQLMQWSSAKVRKYLKEGCELYGLHFLEVPANYTSRQCSRTGLPGIRCDDVPT |
| | GDFLKAPWWRRAINTAREKNGGDAKDRFLVDLYDHLNNLQSKGEALPATVRVPRQGGNLFIAGAQLDDTNK |
| | ERRAIQADLNAAANIGLRALLDPDWRGRWWYVPCKDGTSEPALDRIEGSTAFNDVRSLPTGDNSSRRAPRE |
| | IENLWRDPSGDSLESGTWSPTRAYWDTVQSRVIELLRRHAGLPTS (SEQ ID NO: 127) |
| LsCas12b | MSIRSFKLKLKTKSGVNAEQLRRGLWRTHQLINDGIAYYMNWLVLLRQEDLFIRNKETNEIEKRSKEEIQA |
| Laceyella | VLLERVHKQQQRNQWSGEVDEQTLLQALRQLYEEIVPSVIGKSGNASLKARFFLGPLVDPNNKTTKDVSKS |
| sacchari | GPTPKWKKMKDAGDPNWVQEYEKYMAERQTLVRLEEMGLIPLFPMYTDEVGDIHWLPQASGYTRTWDRDMF |
| WP_13222189 | QQAIERLLSWESWNRRVRERRAQFEKKTHDFASRFSESDVQWMNKLREYEAQQEKSLEENAFAPNEPYALT |
| 4.1 | KKALRGWERVYHSWMRLDSAASEEAYWQEVATCQTAMRGEFGDPAIYQFLAQKENHDIWRGYPERVIDFAE |
| | LNHLQRELRRAKEDATFTLPDSVDHPLWVRYEAPGGTNIHGYDLVQDTKRNLTLILDKFILPDENGSWHEV |
| | KKVPFSLAKSKQFHRQVWLQEEQKQKKREVVFYDYSTNLPHLGTLAGAKLQWDRNFLNKRTQQQ1EETGEI |
| | GKVFFNISVDVRPAVEVKNGRLQNGLGKALTVLTHPDGTKIVTGWKAEQLEKWVGESGRVSSLGLDSLSEG |
| | LRVMSIDLGQRTSATVSVFEITKEAPDNPYKFFYQLEGTEMFAVHQRSFLLALPGENPPQKIKQMREIRWK |
| | ERNRIKQQVDQLSAILRLHKKVNEDERIQAIDKLLQKVASWQLNEEIATAWNQALSQLYSKAKENDLQWNQ |
| | AIKNAHHQLEPVVGKQISLWRKDLSTGRQGIAGLSLWSIEELEATKKLLTRWSKRSREPGVVKRIERFETF |
| | AKQIQHHINQVKENRLKQLANLIVMTALGYKYDQEQKKWIEVYPACQVVLFENLRSYRFSFERSRRENKKL |
| | MEWSHRSIPKLVQMQGELFGLQVADVYAAYSSRYHGRTGAPGIRCHALTEADLRNETNIIHELIEAGFIKE |
| | EHRPYLQQGDLVPWSGGELFATLQKPYDNPRILTLHADINAAQNIQKRFWHPSMWFRVNCESVMEGEIVTY |
| | VPKNKTVHKKQGKTFRFVKVEGSDVYEWAKWSKNRNKNTFSSITERKPPSSMILFRDPSGTFFKEQEWVEQ |
| | KTFWGKVQSMIQAYMKKTIVRQRMEE (SEQ ID NO: 128) |
| DtCas12b | MVLGRKDDTAELRRALWTTHEHVNLAVAEVERVLLRCRGRSYWTLDRRGDPVHVPESQVAEDALAMAREAQ |
| Dsulfonatronum | RRNGWPVVGEDEEILLALRYLYEQIVPSCLLDDLGKPLKGDAQKIGTNYAGPLFDSDTCRRDEGKDVACCG |
| thiodismutans | PFHEVAGKYLGALPEWATPISKQEFDGKDASHLRFKATGGDDAFFRVSIEKANAWYEDPANQDALKNKAYN |
| WP_03138643 | KDDWKKEKDKGISSWAVKYIQKQLQLGQDPRTEVRRKLWLELGLLPLFIPVFDKTMVGNLWNRLAVRLALA |
| 7 | HLLSWESWNHRAVQDQALARAKRDELAALFLGMEDGFAGLREYELRRNESIKQHAFEPVDRPYVVSGRALR |
| | SWTRVREEWLRHGDTQESRKNICNRLQDRLRGKFGDPDVFHWLAEDGQEALWKERDCVTSFSLLNDADGLL |
| | EKRKGYALMTFADARLHPRWAMYEAPGGSNLRTYQIRKTENGLWADVVLLSPRNESAAVEEKTFNVRLAPS |
| | GQLSNVSFDQIQKGSKMVGRCRYQSANQQFEGLLGGAEILFDRKRIANEQHGATDLASKPGHVWFKLTLDV |
| | RPQAPQGWLDGKGRPALPPEAKHFKTALSNKSKFADQVRPGLRVLSVDLGVRSFAACSVFELVRGGPDQGT |
| | YFPAADGRTVDDPEKLWAKHERSFKITLPGENPSRKEEIARRAAMEELRSLNGDIRRLKAILRLSVLQEDD |
| | PRTEHLRLFMEAIVDDPAKSALNAELFKGFGDDRFRSTPDLWKQHCHFFHDKAEKVVAERFSRWRTETRPK |
| | SSSWQDWRERRGYAGGKSYWAVTYLEAVRGLILRWNMRGRTYGEVNRQDKKQFGTVASALLHHINQLKEDR |
| | IKTGADMIIQAARGFVPRKNGAGWVQVHEPCRLILFEDLARYRFRTDRSRRENSRLMRWSHREIVNEVGMQ |
| | GELYGLHVDTTEAGFSSRYLASSGAPGVRCRHLVEEDFHDGLPGMHLVGELDWLLPKDKDRTANEARRLLG |
| | GMVRPGMLVPWDGGELFATLNAASQLHVIHADINAAQNLQRRFWGRCGEAIRIVCNQLSVDGSTRYEMAKA |
| | PKARLLGALQQLKNGDAPFHLTSIPNSQKPENSYVMTPTNAGKKYRAGPGEKSSGEEDELALDIVEQAEEL |
| | AQGRKTFFRDPSGVFFAPDRWLPSEIYWSRIRRRIWQVTLERNSSGRQERAEMDEMPY |
| | (SEQ ID NO: 129) |

The base editors described herein may also comprise Cas12a/Cpf1 (dCpf 1) variants that may be used as a guide nucleotide sequence-programmable DNA-binding protein domain. The Cas12a/Cpf1 protein has a RuvC-like endonuclease domain that is similar to the RuvC domain of Cas9 but does not have a HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity.

(viii) napDNAbps that Recognize Non-Canonical PAM Sequences

In some embodiments, the napDNAbp is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 2016 Jul; 34(7):768-73. PubMed PMID: 27136078; Swarts et al., Nature. 507(7491) (2014):258-61; and Swarts et al., Nucleic Acids Res. 43(10) (2015):5120-9, each of which is incorporated herein by reference.

In some embodiments, the disclosure provides napDNAbp domains that comprise SpCas9 variants that recognize and work best with NRRH, NRCH, and NRTH PAMs. See International Application No. PCT/US2019/47996, which published as International Publication No. WO 2020/041751 on Feb. 27, 2020, incorporated by reference herein. In some embodiments, the disclosed base editors comprise a napDNAbp domain selected from SpCas9-NRRH, SpCas9-NRTH, and SpCas9-NRCH.

In some embodiments, the disclosed base editors comprise a napDNAbp domain that has a sequence that is at least 90%, at least 95%, at least 98%, or at least 99% identical to SpCas9-NRRH. In some embodiments, the disclosed base editors comprise a napDNAbp domain that comprises SpCas9-NRRH. The SpCas9-NRRH has an amino acid sequence as presented in SEQ ID NO: 435 (underligned residues are mutated relative to SpCas9, as set forth in SEQ ID NO: 326):

(SEQ ID NO: 435)
MDKKYSIGLDIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMVKRYDEHHQDLTLLKALVRQQLPEKYKEI
FFDQSKNGYAGYIDGGASQEEFYKFIKPILEKMDG
TEELLVKLNREDLLRKQRTFDNGIIPHQIHLGELH
AILRRQGDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRLRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGGHKPENIVIEMARENQTT
QKGQKNSRERMKRIEEGIKELGSQILKEHPVENTQ
LQNEKLYLYYLQNGRDMYVDQELDINRLSDYDVDH
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKGNSDKLI
ARKKDWDPKKYGGFNSPTAAYSVLVVAKVEKGKSK
KLKSVKELLGITIMERSSFEKNPIGFLEAKGYKEV
KKDLIIKLPKYSLFELENGRKRMLASAGyLHKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGVPAAFKYFDTT
IDKKRYTSTKEVLDATLIHQSITGLYETRIDLSQL
GGD.

In some embodiments, the disclosed base editors comprise a napDNAbp domain that has a sequence that is at least 90%, at least 95%, at least 98%, or at least 99% identical to SpCas9-NRCH. In some embodiments, the disclosed base editors comprise a napDNAbp domain that comprises SpCas9-NRCH. An example of an NRCH PAM is CACC (5′-CACC-3′). The SpCas9-NRCH has an amino acid sequence as presented in SEQ ID NO: 436 (underligned residues are mutated relative to SpCas9):

(SEQ ID NO: 436)
MDKKYSIGLDIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMVKRYDEHHQDLTLLKALVRQQLPEKYKEI
FFDQSKNGYAGYIDGGASQEEFYKFIKPILEKMDG
TEELLVKLNREDLLRKQRTFDNGIIPHQIHLGELH
AILRRQGDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRLRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGGHKPENIVIEMARENQTT
QKGQKNSRERMKRIEEGIKELGSQILKEHPVENTQ
LQNEKLYLYYLQNGRDMYVDQELDINRLSDYDVDH
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKGNSDKLI
ARKKDWDPKKYGGFNSPTVAYSVLVVAKVEKGKSK
KLKSVKELLGITIMERSSFEKNPIDFLEAKGYKEV
KKDLIIKLPKYSLFELENGRKRMLASAGVLQKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGAPAAFKYFDTT
INRKQYNTTKEVLDATLIRQSITGLYETRIDLSQL
GGD

In some embodiments, the disclosed base editors comprise a napDNAbp domain that has a sequence that is at least 90%, at least 95%, at least 98%, or at least 99% identical to SpCas9-NRTH. In some embodiments, the disclosed base editors comprise a napDNAbp domain that comprises SpCas9-NRTH. The SpCas9-NRTH has an amino acid sequence as presented in SEQ ID NO: 437 (underligned residues are mutated relative to SpCas9)

(SEQ ID NO: 437)
MDKKYSIGLDIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMVKRYDEHHQDLTLLKALVRQQLPEKYKEI
FFDQSKNGYAGYIDGGASQEEFYKFIKPILEKMDG
TEELLVKLNREDLLRKQRTFDNGIIPHQIHLGELH
AILRRQGDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRLRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGGHKPENIVIEMARENQTT
QKGQKNSRERMKRIEEGIKELGSQILKEHPVENTQ
LQNEKLYLYYLQNGRDMYVDQELDINRLSDYDVDH
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKGNSDKLI
ARKKDWDPKKYGGFNSPTVAYSVLVVAKVEKGKSK
KLKSVKELLGITIMERSSFEKNPIGFLEAKGYKEV
KKDLIIKLPKYSLFELENGRKRMLASASVLHKGNE
LALPSKYVNFLYLASHYEKLKGSSEDNKQKQLFVE
QHKHYLDEIIEQISEFSKRVILADANLDKVLSAYN
KHRDKPIREQAENIIHLFTLTNLGASAAFKYFDTT
IGRKLYTSTKEVLDATLIHQSITGLYETRIDLSQL
GGD

In some embodiments, the napDNAbp is an argonaute protein. One example of such a nucleic acid programmable DNA binding protein is an Argonaute protein from Natronobacteriurn gregoryi (NgAgo). NgAgo is an ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 2016 Jul.; 34(7):768-73. PubMed PMID: 27136078; Swarts et al., Nature. 507(7491) (2014):258-61; and Swarts et al., Nucleic Acids Res. 43(10) (2015):5120-9, each of which is incorporated herein by reference.

In some embodiments, the napDNAbp is a prokaryotic homolog of an Argonaute protein. Prokaryotic homologs of Argonaute proteins are known and have been described, for example, in Makarova K., et al., “Prokaryotic homologs of Argonaute proteins are predicted to function as key components of a novel system of defense against mobile genetic elements”, Biol Direct. 2009 Aug 25; 4:29. doi: 10.1186/1745-6150-4-29, the entire contents of which is hereby incorporated by reference. In some embodiments, the napDNAbp is a Marinitoga piezophila Argunaute (MpAgo) protein. The CRISPR-associated Marinitoga piezophila Argunaute (MpAgo) protein cleaves single-stranded target sequences using 5′-phosphorylated guides. The 5′ guides are used by all known Argonautes. The crystal structure of an MpAgo-RNA complex shows a guide strand binding site comprising residues that block 5′ phosphate interactions. This data suggests the evolution of an Argonaute subclass with noncanonical specificity for a 5′-hydroxylated guide. See, e.g., Kaya et al., “A bacterial Argonaute with noncanonical guide RNA specificity”, Proc Natl Acad Sci USA. 2016 Apr. 12; 113(15):4057-62, the entire contents of which are hereby incorporated by reference). It should be appreciated that other argonaute proteins may be used, and are within the scope of this disclosure.

In some embodiments, the napDNAbp is a single effector of a microbial CRISPR-Cas system. Single effectors of microbial CRISPR-Cas systems include, without limitation, Cas9, Cpf1, C2c1, C2c2, and C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, C2c1 and C2c3, contain RuvC-like endonuclease domains related to Cpf1. A third system, C2c2 contains an effector with two predicated HEPN RNase domains. Production of mature CRISPR RNA is tracrRNA-independent, unlike production of CRISPR RNA by C2c1. C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cpf1. See, e.g., East-Seletsky, et al., “Two distinct RNase activities of CRISPR-C2c2 enable guide-RNA processing and RNA detection”, Nature, 2016 Oct. 13; 538(7624):270-273, the entire contents of which are hereby incorporated by reference. In vitro biochemical analysis of C2c2 in Leptotrichia shahii has shown that C2c2 is guided by a single CRISPR RNA and can be programed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector”, Science, 2016 Aug. 5; 353(6299), the entire contents of which are hereby incorporated by reference.

The crystal structure of Alicyclobaccillus acidoterrastris C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the napDNAbp may be a C2c1, a C2c2, or a C2c3 protein. In some embodiments, the napDNAbp is a C2c1 protein. In some embodiments, the napDNAbp is a C2c2 protein. In some embodiments, the napDNAbp is a C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring C2c1, C2c2, or C2c3 protein. In some embodiments, the napDNAbp is a naturally-occurring C2c1, C2c2, or C2c3 protein.

Some aspects of the disclosure provide Cas9 domains that have different PAM specificities. Typically, Cas9 proteins, such as Cas9 from S. pyogenes (spCas9), require a canonical NGG PAM sequence to bind a particular nucleic acid region. This may limit the ability to edit desired bases within a genome. In some embodiments, the base editors described herein may need to be placed at a precise location, for example where a target base is placed within a 4 base region (e.g., a “editing window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. Accordingly, in some embodiments, any of the base editors described herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

For example, a napDNAbp domain with altered PAM specificity, such as a domain with at least 80%, at least 85%, at least 90%, at least 95%, or at least 99% sequence identity with wild type Francisella novicida Cpf1 (SEQ ID NO: 130) (D917, E1006, and D1255), which has the following amino acid sequence:

(SEQ ID NO: 130)
MSIYQEFVNKYSLSKTLRFELIPQGKTLENIKARG
LILDDEKRAKDYKKAKQIIDKYHQFFIEEILSSVC
ISEDLLQNYSDVYFKLKKSDDDNLQKDFKSAKDTI
KKQISEYIKDSEKFKNLFNQNLIDAKKGQESDLIL
WLKQSKDNGIELFKANSDITDIDEALEIIKSFKGW
TTYFKGFHENRKNVYSSNDIPTSIIYRIVDDNLPK
FLENKAKYESLKDKAPEAINYEQIKKDLAEELTFD
IDYKTSEVNQRVFSLDEVFEIANFNNYLNQSGITK
FNTIIGGKFVNGENTKRKGINEYINLYSQQINDKT
LKKYKMSVLFKQILSDTESKSFVIDKLEDDSDVVT
TMQSFYEQIAAFKTVEEKSIKETLSLLFDDLKAQK
LDLSKIYFKNDKSLTDLSQQVFDDYSVIGTAVLEY
ITQQIAPKNLDNPSKKEQELIAKKTEKAKYLSLET
IKLALEEFNKHRDIDKQCRFEEILANFAAIPMIFD
EIAQNKDNLAQISIKYQNQGKKDLLQASAEDDVKA
IKDLLDQTNNLLHKLKIFHISQSEDKANILDKDEH
FYLVFEECYFELANIVPLYNKIRNYITQKPYSDEK
FKLNFENSTLANGWDKNKEPDNTAILFIKDDKYYL
GVMNKKNNKIFDDKAIKENKGEGYKKIVYKLLPGA
NKMLPKVFFSAKSIKFYNPSEDILRIRNHSTHTKN
GSPQKGYEKFEFNIEDCRKFIDFYKQSISKHPEWK
DFGFRFSDTQRYNSIDEFYREVENQGYKLTFENIS
ESYIDSVVNQGKLYLFQIYNKDFSAYSKGRPNLHT
LYWKALFDERNLQDVVYKLNGEAELFYRKQSIPKK
ITHPAKEAIANKNKDNPKKESVFEYDLIKDKRFTE
DKFFFHCPITINFKSSGANKFNDEINLLLKEKAND
VHILSIDRGERHLAYYTLVDGKGNIIKQDTFNIIG
NDRMKTNYHDKLAAIEKDRDSARKDWKKINNIKEM
KEGYLSQVVHEIAKLVIEYNAIVVFEDLNFGFKRG
RFKVEKQVYQKLEKMLIEKLNYLVFKDNEFDKTGG
VLRAYQLTAPFETFKKMGKQTGIIYYVPAGFTSKI
CPVTGFVNQLYPKYESVSKSQEFFSKFDKICYNLD
KGYFEFSFDYKNFGDKAAKGKWTIASFGSRLINFR
NSDKNHNWDTREVYPTKELEKLLKDYSIEYGHGEC
IKAAICGESDKKFFAKLTSVLNTILQMRNSKTGTE
LDYLISPVADVNGNFFDSRQAPKNMPQDADANGAY
HIGLKGLMLLGRIKNNQEGKKLNLVIKNEEYFEFV
QNRNN

An additional napDNAbp domain with altered PAM specificity, such as a domain having at least 80%, at least 85%, at least 90%, at least 95%, or at least 99% sequence identity with wild type Geobacillus thermodenitrificans Cas9 (SEQ ID NO: 131), which has the following amino acid sequence:

(SEQ ID NO: 131)
MKYKIGLDIGITSIGWAVINLDIPRIEDLGVRIFD
RAENPKTGESLALPRRLARSARRRLRRRKHRLERI
RRLFVREGILTKEELNKLFEKKHEIDVWQLRVEAL
DRKLNNDELARILLHLAKRRGFRSNRKSERTNKEN
STMLKHIEENQSILSSYRTVAEMVVKDPKFSLHKR
NKEDNYTNTVARDDLEREIKLIFAKQREYGNIVCT
EAFEHEYISIWASQRPFASKDDIEKKVGFCTFEPK
EKRAPKATYTFQSFTVWEHINKLRLVSPGGIRALT
DDERRLIYKQAFHKNKITFHDVRTLLNLPDDTRFK
GLLYDRNTTLKENEKVRFLELGAYHKIRKAIDSVY
GKGAAKSFRPIDFDTFGYALTMFKDDTDIRSYLRN
EYEQNGKRMENLADKVYDEELIEELLNLSFSKFGH
LSLKALRNILPYMEQGEVYSTACERAGYTFTGPKK
KQKTVLLPNIPPIANPVVMRALTQARKVVNAIIKK
YGSPVSIHIELARELSQSFDERRKMQKEQEGNRKK
NETAIRQLVEYGLTLNPTGLDIVKFKLWSEQNGKC
AYSLQPIEIERLLEPGYTEVDHVIPYSRSLDDSYT
NKVLVLTKENREKGNRTPAEYLGLGSERWQQFETF
VLTNKQFSKKKRDRLLRLHYDENEENEFKNRNLND
TRYISRFLANFIREHLKFADSDDKQKVYTVNGRIT
AHLRSRWNFNKNREESNLHHAVDAAIVACTTPSDI
ARVTAFYQRREQNKELSKKTDPQFPQPWPHFADEL
QARLSKNPKESIKALNLGNYDNEKLESLQPVFVSR
MPKRSITGAAHQETLRRYIGIDERSGKIQTVVKKK
LSEIQLDKTGHFPMYGKESDPRTYEAIRQRLLEHN
NDPKKAFQEPLYKPKKNGELGPIIRTIKIIDTTNQ
VIPLNDGKTVAYNSNIVRVDVFEKDGKYYCVPIYT
IDMMKGILPNKAIEPNKPYSEWKEMTEDYTFRFSL
YPNDLIRIEFPREKTIKTAVGEEIKIKDLFAYYQT
IDSSNGGLSLVSHDNNFSLRSIGSRTLKRFEKYQV
DVLGNIYKVRGEKRVGVASSSHSKAGETIRPL

In some embodiments, the nucleic acid programmable DNA binding protein (napDNAbp) is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is an ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 34(7): 768-73 (2016), PubMed PMID: 27136078; Swarts et al., Nature, 507(7491): 258-61 (2014); and Swarts et al., Nucleic Acids Res. 43(10) (2015): 5120-9, each of which is incorporated herein by reference. The sequence of Natronobacterium gregoryi Argonaute is provided in SEQ ID NO: 132.

The disclosed base editors may comprise a napDNAbp domain having at least 80%, at least 85%, at least 90%, at least 95%, or at least 99% sequence identity with wild type Natronobacterium gregoryi Argonaute (SEQ ID NO: 132), which has the following amino acid sequence:

(SEQ ID NO: 132)
MTVIDLDSTTTADELTSGHTYDISVTLTGVYDNTD
EQHPRMSLAFEQDNGERRYITLWKNTTPKDVFTYD
YATGSTYIFTNIDYEVKDGYENLTATYQTTVENAT
AQEVGTTDEDETFAGGEPLDHHLDDALNETPDDAE
TESDSGHVMTSFASRDQLPEWTLHTYTLTATDGAK
TDTEYARRTLAYTVRQELYTDHDAAPVATDGLMLL
TPEPLGETPLDLDCGVRVEADETRTLDYTTAKDRL
LARELVEEGLKRSLWDDYLVRGIDEVLSKEPVLTC
DEFDLHERYDLSVEVGHSGRAYLHINFRHRFVPKL
TLADIDDDNIYPGLRVKTTYRPRRGHIVWGLRDEC
ATDSLNTLGNQSVVAYHRNNQTPINTDLLDAIEAA
DRRVVETRRQGHGDDAVSFPQELLAVEPNTHQIKQ
FASDGFHQQARSKTRLSASRCSEKAQAFAERLDPV
RLNGSTVEFSSEFFTGNNEQQLRLLYENGESVLTF
RDGARGAHPDETFSKGIVNPPESFEVAVVLPEQQA
DTCKAQWDTMADLLNQAGAPPTRSETVQYDAFSSP
ESISLNVAGAIDPSEVDAAFVVLPPDQEGFADLAS
PTETYDELKKALANMGIYSQMAYFDRFRDAKIFYT
RNVALGLLAAAGGVAFTTEHAMPGDADMFIGIDVS
RSYPEDGASGQINIAATATAVYKDGTILGHSSTRP
QLGEKLQSTDVRDIMKNAILGYQQVTGESPTHIVI
HRDGFMNEDLDPATEFLNEQGVEYDIVEIRKQPQT
RLLAVSDVQYDTPVKSIAAINQNEPRATVATFGAP
EYLATRDGGGLPRPIQIERVAGETDIETLTRQVYL
LSQSHIQVHNSTARLPITTAYADQASTHATKGYLV
QTGAFESNVGFL

(ix) Cas9 Variants with Modified PAM Specificities

The base editors of the present disclosure may also comprise Cas9 variants with modified PAM specificities. Some aspects of this disclosure provide Cas9 proteins that exhibit activity on a target sequence that does not comprise the canonical PAM (5′-NGG-3′, where N is A, C, G, or T) at its 3′-end. In some embodiments, the Cas9 protein exhibits activity on a target sequence comprising a 5′-NGG-3′ PAM sequence at its 3′-end. In some embodiments, the Cas9 protein exhibits activity on a target sequence comprising a 5′-NNG-3′ PAM sequence at its 3′-end. In some embodiments, the Cas9 protein exhibits activity on a target sequence comprising a 5′-NNA-3′ PAM sequence at its 3′-end. In some embodiments, the Cas9 protein exhibits activity on a target sequence comprising a 5′-NNC-3′ PAM sequence at its 3′-end. In some embodiments, the Cas9 protein exhibits activity on a target sequence comprising a 5′-NNT-3′ PAM sequence at its 3′-end. In some embodiments, the Cas9 protein exhibits activity on a target sequence comprising a 5′-NGT-3′ PAM sequence at its 3′-end. In some embodiments, the Cas9 protein exhibits activity on a target sequence comprising a 5′-NGA-3′ PAM sequence at its 3′-end. In some embodiments, the Cas9 protein exhibits activity on a target sequence comprising a 5′-NGC-3′ PAM sequence at its 3′-end. In some embodiments, the Cas9 protein exhibits activity on a target sequence comprising a 5′-NAA-3′ PAM sequence at its 3′-end. In some embodiments, the Cas9 protein exhibits activity on a target sequence comprising a 5′-NAC-3′ PAM sequence at its 3′-end. In some embodiments, the Cas9 protein exhibits activity on a target sequence comprising a 5′-NAT-3′ PAM sequence at its 3′-end. In still other embodiments, the Cas9 protein exhibits activity on a target sequence comprising a 5′-NAG-3′ PAM sequence at its 3′-end.

In some embodiments, the disclosed adenine base editors comprise a napDNAbp domain comprising a SpCas9-NG, which has a PAM that corresponds to NGN. In some embodiments, the disclosed base editors comprise a napDNAbp domain that has a sequence that is at least 90%, at least 95%, at least 98%, or at least 99% identical to SpCas9-NG. The seauence of SpCas9-NG is illustrated below:

(SEQ ID NO: 153)
MDKKYSIGLAIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMIKRYDEHHQDLTLLKALVRQQLPEKYKEI
FFDQSKNGYAGYIDGGASQEEFYKFIKPILEKMDG
TEELLVKLNREDLLRKQRTFDNGSIPHQIHLGELH
AILRRQEDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRRRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGRHKPENIVIEMARENQTT
QKGQKNSRERMKRIEEGIKELGSQILKEHPVENTQ
LQNEKLYLYYLQNGRDMYVDQELDINRLSDYDVDH
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESIRPKRNSDKLI
ARKKDWDPKKYGGFVSPTVAYSVLVVAKVEKGKSK
KLKSVKELLGITIMERSSFEKNPIDFLEAKGYKEV
KKDLIIKLPKYSLFELENGRKRMLASARFLQKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGAPRAFKYFDTT
IDRKVYRSTKEVLDATLIHQSITGLYETRIDLSQL
GGD

In some embodiments, the disclosed base editors comprise a napDNAbp domain comprising a SaCas9-KKH, which has a PAM that corresponds to NNNRRT. This Cas9 variant contains the amino acid substitutions DlOA, E782K, N968K, and R1015H relative to wild-type SaCas9. In some embodiments, the disclosed base editors comprise a napDNAbp domain that has a sequence that is at least 90%, at least 95%, at least 98%, or at least 99% identical to SaCas9-KKH. The sequence of SaCas9-KKH is illustrated below: S. aureus Cas9 nickase KKH (D10A/E782K/N968K/R1015H) (SaCas9-KKH)

(SEQ ID NO: 154)
MGKRNYILGLAIGITSVGYGIIDYETRDVIDAGVR
LFKEANVENNEGRRSKRGARRLKRRRRHRIQRVKK
LLFDYNLLTDHSELSGINPYEARVKGLSQKLSEEE
FSAALLHLAKRRGVHNVNEVEEDTGNELSTKEQIS
RNSKALEEKYVAELQLERLKKDGEVRGSINRFKTS
DYVKEAKQLLKVQKAYHQLDQSFIDTYIDLLETRR
TYYEGPGEGSPFGWKDIKEWYEMLMGHCTYFPEEL
RSVKYAYNADLYNALNDLNNLVITRDENEKLEYYE
KFQIIENVFKQKKKPTLKQIAKEILVNEEDIKGYR
VTSTGKPEFTNLKVYHDIKDITARKEIIENAELLD
QIAKILTIYQSSEDIQEELTNLNSELTQEEIEQIS
NLKGYTGTHNLSLKAINLILDELWHTNDNQIAIFN
RLKLVPKKVDLSQQKEIPTTLVDDFILSPVVKRSF
IQSIKVINAIIKKYGLPNDIIIELAREKNSKDAQK
MINEMQKRNRQTNERIEEIIRTTGKENAKYLIEKI
KLHDMQEGKCLYSLEAIPLEDLLNNPFNYEVDHII
PRSVSFDNSFNNKVLVKQEENSKKGNRTPFQYLSS
SDSKISYETFKKHILNLAKGKGRISKTKKEYLLEE
RDINRFSVQKDFINRNLVDTRYATRGLMNLLRSYF
RVNNLDVKVKSINGGFTSFLRRKWKFKKERNKGYK
HHAEDALIIANADFIFKEWKKLDKAKKVMENQMFE
EKQAESMPEIETEQEYKEIFITPHQIKHIKDFKDY
KYSHRVDKKPNRKLINDTLYSTRKDDKGNTLIVNN
LNGLYDKDNDKLKKLINKSPEKLLMYHHDPQTYQK
LKLIMEQYGDEKNPLYKYYEETGNYLTKYSKKDNG
PVIKKIKYYGNKLNAHLDITDDYPNSRNKVVKLSL
KPYRFDVYLDNGVYKFVTVKNLDVIKKENYYEVNS
KCYEEAKKLKKISNQAEFIASFYKNDLIKINGELY
RVIGVNNDLLNRIEVNMIDITYREYLENMNDKRPP
HIIKTIASKTQSIKKYSTDILGNLYEVKSKKHPQI
IKKG

In some embodiments, the disclosed adenine base editors comprise a napDNAbp domain comprising a xCas9, an evolved variant of SpCas9. In some embodiments, the disclosed base editors comprise a napDNAbp domain that has a sequence that is at least 90%, at least 95%, at least 98%, or at least 99% identical to xCas9. The sequence of xCas9 is illustrated below:

(SEQ ID NO: 155)
MDKKYSIGLAIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDTKLQLSKDTYDDDLDNLLA
QIGDQYADLFLAAKNLSDAILLSDILRVNTEITKA
PLSASMIKLYDEHHQDLTLLKALVRQQLPEKYKEI
FFDQSKNGYAGYIDGGASQEEFYKFIKPILEKMDG
TEELLVKLNREDLLRKQRTFDNGIIPHQIHLGELH
AILRRQEDFYPFLKDNREKIEKILTFRIPYYVGPL
ARGNSRFAWMTRKSEETITPWNFEKVVDKGASAQS
FIERMTNFDKNLPNEKVLPKHSLLYEYFTVYNELT
KVKYVTEGMRKPAFLSGDQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRRRYTGWGRLSRK
LINGIRDKQSGKTILDFLKSDGFANRNFIQLIHDD
SLTFKEDIQKAQVSGQGDSLHEHIANLAGSPAIKK
GILQTVKVVDELVKVMGRHKPENIVIEMARENQTT
QKGQKNSRERMKRIEEGIKELGSQILKEHPVENTQ
LQNEKLYLYYLQNGRDMYVDQELDINRLSDYDVDH
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKRNSDKLI
ARKKDWDPKKYGGFDSPTVAYSVLVVAKVEKGKSK
KLKSVKELLGITIMERSSFEKNPIDFLEAKGYKEV
KKDLIIKLPKYSLFELENGRKRMLASAGVLQKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGAPAAFKYFDTT
IDRKRYTSTKEVLDATLIHQSITGLYETRIDLSQL
GGD

It should be appreciated that any of the amino acid mutations described herein, (e.g., A262T) from a first amino acid residue (e.g., A) to a second amino acid residue (e.g., T) may also include mutations from the first amino acid residue to an amino acid residue that is similar to (e.g., conserved) the second amino acid residue. For example, mutation of an amino acid with a hydrophobic side chain (e.g., alanine, valine, isoleucine, leucine, methionine, phenylalanine, tyrosine, or tryptophan) may be a mutation to a second amino acid with a different hydrophobic side chain (e.g., alanine, valine, isoleucine, leucine, methionine, phenylalanine, tyrosine, or tryptophan). For example, a mutation of an alanine to a threonine (e.g., a A262T mutation) may also be a mutation from an alanine to an amino acid that is similar in size and chemical properties to a threonine, for example, serine. As another example, mutation of an amino acid with a positively charged side chain (e.g., arginine, histidine, or lysine) may be a mutation to a second amino acid with a different positively charged side chain (e.g., arginine, histidine, or lysine). As another example, mutation of an amino acid with a polar side chain (e.g., serine, threonine, asparagine, or glutamine) may be a mutation to a second amino acid with a different polar side chain (e.g., serine, threonine, asparagine, or glutamine). Additional similar amino acid pairs include, but are not limited to, the following: phenylalanine and tyrosine; asparagine and glutamine; methionine and cysteine; aspartic acid and glutamic acid; and arginine and lysine. The skilled artisan would recognize that such conservative amino acid substitutions will likely have minor effects on protein structure and are likely to be well tolerated without compromising function. In some embodiments, any amino of the amino acid mutations provided herein from one amino acid to a threonine may be an amino acid mutation to a serine. In some embodiments, any amino of the amino acid mutations provided herein from one amino acid to an arginine may be an amino acid mutation to a lysine. In some embodiments, any amino of the amino acid mutations provided herein from one amino acid to an isoleucine, may be an amino acid mutation to an alanine, valine, methionine, or leucine. In some embodiments, any amino of the amino acid mutations provided herein from one amino acid to a lysine may be an amino acid mutation to an arginine. In some embodiments, any amino of the amino acid mutations provided herein from one amino acid to an aspartic acid may be an amino acid mutation to a glutamic acid or asparagine. In some embodiments, any amino of the amino acid mutations provided herein from one amino acid to a valine may be an amino acid mutation to an alanine, isoleucine, methionine, or leucine. In some embodiments, any amino of the amino acid mutations provided herein from one amino acid to a glycine may be an amino acid mutation to an alanine. It should be appreciated, however, that additional conserved amino acid residues would be recognized by the skilled artisan and any of the amino acid mutations to other conserved amino acid residues are also within the scope of this disclosure.

In some embodiments, the present disclosure may utilize any of the Cas9 variants disclosed in the SEQUENCES section herein.

In some embodiments, the Cas9 protein comprises a combination of mutations that exhibit activity on a target sequence comprising a 5′-NAA-3′ PAM sequence at its 3′-end. In some embodiments, the combinations of mutations are present in any one of the clones listed in Table 1. In some embodiments, the combinations of mutations are conservative mutations of the clones listed in Table 1. In some embodiments, the Cas9 protein comprises the combination of mutations of any one of the Cas9 clones listed in Table 1.

TABLE 1
NAA PAM Clones
Mutations from wild-type SpCas9 (e.g., SEQ ID NO: )

D177N, K218R, D614N, D1135N, P1137S, E1219V, A1320V, A1323D, R1333K
D177N, K218R, D614N, D1135N, E1219V, Q1221H, H1264Y, A1320V, R1333K
A10T, I322V, S409I, E427G, G715C, D1135N, E1219V, Q1221H, H1264Y, A1320V, R1333K
A367T, K710E, R1114G, D1135N, P1137S, E1219V, Q1221H, H1264Y, A1320V, R1333K
A10T, I322V, S409I, E427G, R753G, D861N, D1135N, K1188R, E1219V, Q1221H, H1264H,
A1320V, R1333K
A10T, I322V, S409I, E427G, R654L, V743I, R753G, M1021T, D1135N, D1180G, K1211R,
E1219V, Q1221H, H1264Y, A1320V, R1333K
A10T, I322V, S409I, E427G, V743I, R753G, E762G, D1135N, D1180G, K1211R, E1219V,
Q1221H, H1264Y, A1320V, R1333K
A10T, I322V, S409I, E427G, R753G, D1135N, D1180G, K1211R, E1219V, Q1221H, H1264Y,
S1274R, A1320V, R1333K
A10T, I322V, S409I, E427G, A589S, R753G, D1135N, E1219V, Q1221H, H1264H, A1320V,
R1333K
A10T, I322V, S409I, E427G, R753G, E757K, G865G, D1135N, E1219V, Q1221H, H1264Y,
A1320V, R1333K
A10T, I322V, S409I, E427G, R654L, R753G, E757K, D1135N, E1219V, Q1221H, H1264Y,
A1320V, R1333K
A10T, I322V, S409I, E427G, K599R, M631A, R654L, K673E, V743I, R753G, N758H, E762G,
D1135N, D1180G, E1219V, Q1221H, Q1256R, H1264Y, A1320V, A1323D, R1333K
A10T, I322V, S409I, E427G, R654L, K673E, V743I, R753G, E762G, N869S, N1054D, R1114G,
D1135N, D1180G, E1219V, Q1221H, H1264Y, A1320V, A1323D, R1333K
A10T, I322V, S409I, E427G, R654L, L727I, V743I, R753G, E762G, R859S, N946D, F1134L,
D1135N, D1180G, E1219V, Q1221H, H1264Y, N1317T, A1320V, A1323D, R1333K
A10T, I322V, S409I, E427G, R654L, K673E, V743I, R753G, E762G, N803S, N869S, Y1016D,
G1077D, R1114G, F1134L, D1135N, D1180G, E1219V, Q1221H, H1264Y, V1290G, L1318S,
A1320V, A1323D, R1333K
A10T, I322V, S409I, E427G, R654L, K673E, V743I, R753G, E762G, N803S, N869S, Y1016D,
G1077D, R1114G, F1134L, D1135N, K1151E, D1180G, E1219V, Q1221H, H1264Y, V1290G,
L1318S, A1320V, R1333K
A10T, I322V, S409I, E427G, R654L, K673E, V743I, R753G, E762G, N803S, N869S, Y1016D,
G1077D, R1114G, F1134L, D1135N, D1180G, E1219V, Q1221H, H1264Y, V1290G, L1318S,
A1320V, A1323D, R1333K
A10T, I322V, S409I, E427G, R654L, K673E, F693L, V743I, R753G, E762G, N803S, N869S,
L921P, Y1016D, G1077D, F1080S, R1114G, D1135N, D1180G, E1219V, Q1221H, H1264Y,
L1318S, A1320V, A1323D, R1333K
A10T, I322V, S409I, E427G, E630K, R654L, K673E, V743I, R753G, E762G, Q768H, N803S,
N869S, Y1016D, G1077D, R1114G, F1134L, D1135N, D1180G, E1219V, Q1221H, H1264Y,
L1318S, A1320V, R1333K
A10T, I322V, S409I, E427G, R654L, K673E, F693L, V743I, R753G, E762G, Q768H, N803S,
N869S, Y1016D, G1077D, R1114G, F1134L, D1135N, D1180G, E1219V, Q1221H, G1223S,
H1264Y, L1318S, A1320V, R1333K
A10T, I322V, S409I, E427G, R654L, K673E, F693L, V743I, R753G, E762G, N803S, N869S,
L921P, Y1016D, G1077D, F1801S, R1114G, D1135N, D1180G, E1219V, Q1221H, H1264Y,
L1318S, A1320V, A1323D, R1333K
A10T, I322V, S409I, E427G, R654L, V743I, R753G, M1021T, D1135N, D1180G, K1211R,
E1219V, Q1221H, H1264Y, A1320V, R1333K
A10T, I322V, S409I, E427G, R654L, K673E, V743I, R753G, E762G, M673I, N803S, N869S,
G1077D, R1114G, D1135N, V1139A, D1180G, E1219V, Q1221H, A1320V, R1333K
A10T, I322V, S409I, E427G, R654L, K673E, V743I, R753G, E762G, N803S, N869S, R1114G,
D1135N, E1219V, Q1221H, A1320V, R1333K

In some embodiments, the Cas9 protein comprises an amino acid sequence that is at least 80% identical to the amino acid sequence of a Cas9 protein as provided by any one of the variants of Table 1. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table 1.

In some embodiments, the Cas9 protein exhibits an increased activity on a target sequence that does not comprise the canonical PAM (5′-NGG-3′) at its 3′ end as compared to Streptococcus pyogenes Cas9 as provided by SEQ ID NO: 74. In some embodiments, the Cas9 protein exhibits an activity on a target sequence having a 3′ end that is not directly adjacent to the canonical PAM sequence (5′-NGG-3′) that is at least 5-fold increased as compared to the activity of Streptococcus pyogenes Cas9 as provided by SEQ ID NO: 74 on the same target sequence. In some embodiments, the Cas9 protein exhibits an activity on a target sequence that is not directly adjacent to the canonical PAM sequence (5′-NGG-3′) that is at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes as provided by SEQ ID NO: 74 on the same target sequence. In some embodiments, the 3′ end of the target sequence is directly adjacent to an AAA, GAA, CAA, or TAA sequence. In some embodiments, the Cas9 protein comprises a combination of mutations that exhibit activity on a target sequence comprising a 5⁻-NAC-3′ PAM sequence at its 3′-end. In some embodiments, the combinations of mutations are present in any one of the clones listed in Table 2. In some embodiments, the combinations of mutations are conservative mutations of the clones listed in Table 2. In some embodiments, the Cas9 protein comprises the combination of mutations of any one of the Cas9 clones listed in Table 2.

TABLE 2
NAC PAM Clones
MUTATIONS FROM WILD-TYPE SPCAS9 (E.G. , SEQ ID NO 74)

T472I, R753G, K890E, D1332N, R1335Q, T1337N
I1057S, D1135N, P1301S, R1335Q, T1337N
T472I, R753G, D1332N, R1335Q, T1337N
D1135N, E1219V, D1332N, R1335Q, T1337N
T472I, R753G, K890E, D1332N, R1335Q, T1337N
I1057S, D1135N, P1301S, R1335Q, T1337N
T472I, R753G, D1332N, R1335C, T1337N
T472I, R753G, Q771H, D1332N, R1335Q, T1337N
E627K, T638P, K652T, R753G, N803S, K959N, R1114G, D1135N, E1219V, D1332N, R1335Q,
T1337N
E627K, T638P, K652T, R753G, N803S, K959N, R1114G, D1135N, K1156E, E1219V, D1332N,
R1335Q, T1337N
E627K, T638P, V647I, R753G, N803S, K959N, G1030R, I1055E, R1114G, D1135N, E1219V,
D1332N, R1335Q, T1337N
E627K, E630G, T638P, V647A, G687R, N767D, N803S, K959N, R1114G, D1135N, E1219V,
D1332G, R1335Q, T1337N
E627K, T638P, R753G, N803S, K959N, R1114G, D1135N, E1219V, N1266H, D1332N, F1335Q,
T1337N
E627K, T638P, R753G, N803S, K959N, I1057T, R1114G, D1135N, E1219V, D1332N, R1335Q,
T1337N
E627K, T638P, R753G, N803S, K959N, R1114G, D1135N, E1219V, D1332N, R1335Q, T1337N
E627K, M631I, T638P, R753G, N803S, K959N, Y1036H, R1114G, D1135N, E1219V, D1251G,
D1332G, R1335Q, T1337N
E627K, T638P, R753G, N803S, V875I, K959N, Y1016C, R1114G, D1135N, E1219V, D1251G,
D1332G, R1335Q, T1337N, I1348V
K608R, E627K, T638P, V647I, R654L, R753G, N803S, T804A, K848N, V922A, K959N, R1114G,
D1135N, E1219V, D1332N, R1335Q, T1337N
K608R, E627K, T638P, V647I, R753G, N803S, V922A, K959N, K1014N, V1015A, R1114G,
D1135N, K1156N, E1219V, N1252D, D1332N, R1335Q, T1337N
K608R, E627K, R629G, T638P, V647I, A711T, R753G, K775R, K789E, N803S, K959N, V1015A,
Y1036H, R1114G, D1135N, E1219V, N1286H, D1332N, R1335Q, T1337N
K608R, E627K, T638P, V647I, T740A, R753G, N803S, K948E, K959N, Y1016S, R1114G,
D1135N, E1219V, N1286H, D1332N, R1335Q, T1337N
K608R, E627K, T638P, V647I, T740A, N803S, K948E, K959N, Y1016S, R1114G, D1135N,
E1219V, N1286H, D1332N, R1335Q, T1337N
I670S, K608R, E627K, E630G, T638P, V647I, R653K, R753G, I795L, K797N, N803S, K866R,
K890N, K959N, Y1016C, R1114G, D1135N, E1219V, D1332N, R1335Q, I1337N
K608R, E627K, T638P, V647I, T740A, G752R, R753G, K797N, N803S, K948E, K959N, V1015A,
Y1016S, R1114G, D1135N, E1219V, N1266H, D1332N, R1335Q, T1337N
I570T, A589V, K608R, E627K, T638P, V647I, R654L, Q716R, R753G, N803S, K948E, K959N,
Y1016S, R1114G, D1135N, E1207G, E1219V, N1234D, D1332N, R1335Q, T1337N
K608R, E627K, R629G, T638P, V647I, R654L, Q740R, R753G, N803S, K959N, N990S, T995S,
V1015A, Y1036D, R1114G, D1135N, E1207G, E1219V, N1234D, N1266H, D1332N, R1335Q,
T1337N
I562F, V565D, I570T, K608R, L625S, E627K, T638P, V647I, R654I, G752R, R753G, N803S,
N808D, K959N, M1021L, R1114G, D1135N, N1177S, N1234D, D1332N, R1335Q, T1337N
I562F, I570T, K608R, E627K, T638P, V647I, R753G, E790A, N803S, K959N, V1015A, Y1036H,
R1114G, D1135N, D1180E, A1184T, E1219V, D1332N, R1335Q, T1337N
I570T, K608R, E627K, T638P, V647I, R654H, R753G, E790A, N803S, K959N, V1015A, R1114G,
D1127A, D1135N, E1219V, D1332N, R1335Q, T1337N
I570T, K608R, L625S, E627K, T638P, V647I, R654I, T703P, R753G, N803S, N808D, K959N,
M1021L, R1114G, D1135N, E1219V, D1332N, R1335Q, T1337N
I570S, K608R, E627K, E630G, T638P, V647I, R653K, R753G, I795L, N803S, K866R, K890N,
K959N, Y1016C, R1114G, D1135N, E1219V, D1332N, R1335Q, T1337N
I570T, K608R, E627K, T638P, V647I, R654H, R753G, E790A, N803S, K959N, V1016A, R1114G,
D1135N, E1219V, K1246E, D1332N, R1335Q, T1337N
K608R, E627K, T638P, V647I, R654L, K673E, R753G, E790A, N803S, K948E, K959N, R1114G,
D1127G, D1135N, D1180E, E1219V, N1286H, D1332N, R1335Q, T1337N
K608R, L625S, E627K, T638P, V647I, R654I, I670T, R753G, N803S, N808D, K959N, M1021L,
R1114G, D1135N, E1219V, N1286H, D1332N, R1335Q, T1337N
E627K, M631V, T638P, V647I, K710E, R753G, N803S, N808D, K948E, M1021L, R1114G,
D1135N, E1219V, D1332N, R1335Q, T1337N, S1338T, H1349R

In some embodiments, the Cas9 protein comprises an amino acid sequence that is at least 80% identical to the amino acid sequence of a Cas9 protein as provided by any one of the variants of Table 2. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table 2.

In some embodiments, the Cas9 protein exhibits an increased activity on a target sequence that does not comprise the canonical PAM (5′-NGG-3′) at its 3′ end as compared to Streptococcus pyogenes Cas9 as provided by SEQ ID NO: 74. In some embodiments, the Cas9 protein exhibits an activity on a target sequence having a 3′ end that is not directly adjacent to the canonical PAM sequence (5′-NGG-3′) that is at least 5-fold increased as compared to the activity of Streptococcus pyogenes Cas9 as provided by SEQ ID NO: 74 on the same target sequence. In some embodiments, the Cas9 protein exhibits an activity on a target sequence that is not directly adjacent to the canonical PAM sequence (5′-NGG-3′) that is at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes as provided by SEQ ID NO: 74 on the same target sequence. In some embodiments, the 3′ end of the target sequence is directly adjacent to an AAC, GAC, CAC, or TAC sequence.

In some embodiments, the Cas9 protein comprises a combination of mutations that exhibit activity on a target sequence comprising a 5′-NAT-3′ PAM sequence at its 3′-end. In some embodiments, the combinations of mutations are present in any one of the clones listed in Table 3. In some embodiments, the combinations of mutations are conservative mutations of the clones listed in Table 3. In some embodiments, the Cas9 protein comprises the combination of mutations of any one of the Cas9 clones listed in Table 3.

TABLE 3
NAT PAM Clones
MUTATIONS FROM WILD-TYPE SPCAS9 (E.G. , SEQ ID NO: 74)

K961E, H985Y, D1135N, K1191N, E1219V, Q1221H, A1320A, P1321S, R1335L
D1135N, G1218S, E1219V, Q1221H, P1249S, P1321S, D1322G, R1335L
V743I, R753G, E790A, D1135N, G1218S, E1219V, Q1221H, A1227V, P1249S, N1286K, A1293T,
P1321S, D1322G, R1335L, T1339I
F575S, M631L, R654L, V748I, V743I, R753G, D853E, V922A, R1114G, D1135N, G1218S,
E1219V, Q1221H, A1227V, P1249S, N1286K, A1293T, P1321S, D1322G, R1335L, T1339I
F575S, M631L, R654L, R664K, R753G, D853E, V922A, R1114G, D1135N, D1180G, G1218S,
E1219V, Q1221H, P1249S, N1286K, P1321S, D1322G, R1335L
M631L, R654L, R753G, K797E, D853E, V922A, D1012A, R1114G, D1135N, G1218S, E1219V,
Q1221H, P1249S, N1317K, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, D596Y, M631L, R654L, R664K, R753G, D853E, V922A, R1114G, Y1131C, D1135N,
D1180G, G1218S, E1219V, Q1221H, P1249S, Q1256R, P1321S, D1322G, R1335L
F575S, M631L, R654L, R664K, K710E, V750A, R753G, D853E, V922A, R1114G, Y1131C,
D1135N, D1180G, G1218S, E1219V, Q1221H, P1249S, P1321S, D1322G, R1335L
F575S, M631L, K649R, R654L, R664K, R753G, D853E, V922A, R1114G, Y1131C, D1135N,
K1156E, D1180G, G1218S, E1219V, Q1221H, P1249S, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, M631L, R654L, R664K, R753G, D853E, V922A, I1057G, R1114G, Y1131C, D1135N,
D1180G, G1218S, E1219V, Q1221H, P1249S, N1308D, P1321S, D1322G, R1335L
M631L, R654L, R753G, D853E, V922A, R1114G, Y1131C, D1135N, E1150V, D1180G, G1218S,
E1219V, Q1221H, P1249S, P1321S, D1332G, R1335L
M631L, R654L, R664K, R753G, D853E, I1057V, Y1131C, D1135N, D1180G, G1218S, E1219V,
Q1221H, P1249S, P1321S, D1332G, R1335L
M631L, R654L, R664K, R753G, I1057V, R1114G, Y1131C, D1135N, D1180G, G1218S, E1219V,
Q1221H, P1249S, P1321S, D1332G, R1335L

The above description of various napDNAbps which can be used in connection with the presently disclose base editors is not meant to be limiting in any way. The base editors may comprise the canonical SpCas9, or any ortholog Cas9 protein, or any variant Cas9 protein—including any naturally occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave of strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats). The base editors described herein may also comprise Cas9 equivalents, including Cas12a/Cpf1 and Cas12b proteins which are the result of convergent evolution. The napDNAbps used herein (e.g., SpCas9, Cas9 variant, or Cas9 equivalents) may also may also contain various modifications that alter/enhance their PAM specificities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a reference SpCas9 canonical sequences or a reference Cas9 equivalent (e.g., Cas12a/Cpf1).

In a particular embodiment, the Cas9 variant having expanded PAM capabilities is SpCas9 (H840A) VRQR, having the following amino acid sequence (with the V, R, Q, R substitutions relative to the SpCas9 (H840A) of SEQ ID NO: 107, shown in bold underline. In some embodiments, the disclosed base editors comprise a napDNAbp domain that comprises SpCas9-VRQR. In addition, the methionine residue in SpCas9 (H840) was removed for SpCas9 (H840A) VRQR):

(SEQ ID NO: 133)
DKKYSIGLDIGTNSVGWAVITDEYKVPSKKFKVLG
NTDRHSIKKNLIGALLFDSGETAEATRLKRTARRR
YTRRKNRICYLQEIFSNEMAKVDDSFFHRLEESFL
VEEDKKHERHPIFGNIVDEVAYHEKYPTIYHLRKK
LVDSTDKADLRLIYLALAHMIKFRGHFLIEGDLNP
DNSDVDKLFIQLVQTYNQLFEENPINASGVDAKAI
LSARLSKSRRLENLIAQLPGEKKNGLFGNLIALSL
GLTPNFKSNFDLAEDAKLQLSKDTYDDDLDNLLAQ
IGDQYADLFLAAKNLSDAILLSDILRVNTEITKAP
LSASMIKRYDEHHQDLTLLKALVRQQLPEKYKEIF
FDQSKNGYAGYIDGGASQEEFYKFIKPILEKMDGT
EELLVKLNREDLLRKQRTFDNGSIPHQIHLGELHA
ILRRQEDFYPFLKDNREKIEKILTFRIPYYVGPLA
RGNSRFAWMTRKSEETITPWNFEEVVDKGASAQSF
IERMTNFDKNLPNEKVLPKHSLLYEYFTVYNELTK
VKYVTEGMRKPAFLSGEQKKAIVDLLFKTNRKVTV
KQLKEDYFKKIECFDSVEISGVEDRFNASLGTYHD
LLKIIKDKDFLDNEENEDILEDIVLTLTLFEDREM
IEERLKTYAHLFDDKVMKQLKRRRYTGWGRLSRKL
INGIRDKQSGKTILDFLKSDGFANRNFMQLIHDDS
LTFKEDIQKAQVSGQGDSLHEHIANLAGSPAIKKG
ILQTVKVVDELVKVMGRHKPENIVIEMARENQTTQ
KGQKNSRERMKRIEEGIKELGSQILKEHPVENTQL
QNEKLYLYYLQNGRDMYVDQELDINRLSDYDVDAI
VPQSFLKDDSIDNKVLTRSDKNRGKSDNVPSEEVV
KKMKNYWRQLLNAKLITQRKFDNLTKAERGGLSEL
DKAGFIKRQLVETRQITKHVAQILDSRMNTKYDEN
DKLIREVKVITLKSKLVSDFRKDFQFYKVREINNY
HHAHDAYLNAVVGTALIKKYPKLESEFVYGDYKVY
DVRKMIAKSEQEIGKATAKYFFYSNIMNFFKTEIT
LANGEIRKRPLIETNGETGEIVWDKGRDFATVRKV
LSMPQVNIVKKTEVQTGGFSKESILPKRNSDKLIA
RKKDWDPKKYGGFVSPTVAYSVLVVAKVEKGKSKK
LKSVKELLGITIMERSSFEKNPIDFLEAKGYKEVK
KDLIIKLPKYSLFELENGRKRMLASARELQKGNEL
ALPSKYVNFLYLASHYEKLKGSPEDNEQKQLFVEQ
HKHYLDEIIEQISEFSKRVILADANLDKVLSAYNK
HRDKPIREQAENIIHLFTLTNLGAPAAFKYFDTTI
DRKQYRSTKEVLDATLIHQSITGLYETRIDLSQLG
GD

In another particular embodiment, the Cas9 variant having expanded PAM capabilities is SpCas9 (H840A) VRER, having the following amino acid sequence (with the V, R, E, R substitutions relative to the SpCas9 (H840A) of SEQ ID NO: 107 are shown in bold underline. In addition, the methionine residue in SpCas9 (H840) was removed for SpCas9 (H840A) VRER):

(SEQ ID NO: 134)
DKKYSIGLDIGTNSVGWAVITDEYKVPSKKFKVLG
NTDRHSIKKNLIGALLFDSGETAEATRLKRTARRR
YTRRKNRICYLQEIFSNEMAKVDDSFFHRLEESFL
VEEDKKHERHPIFGNIVDEVAYHEKYPTIYHLRKK
LVDSTDKADLRLIYLALAHMIKFRGHFLIEGDLNP
DNSDVDKLFIQLVQTYNQLFEENPINASGVDAKAI
LSARLSKSRRLENLIAQLPGEKKNGLFGNLIALSL
GLTPNFKSNFDLAEDAKLQLSKDTYDDDLDNLLAQ
IGDQYADLFLAAKNLSDAILLSDILRVNTEITKAP
LSASMIKRYDEHHQDLTLLKALVRQQLPEKYKEIF
FDQSKNGYAGYIDGGASQEEFYKFIKPILEKMDGT
EELLVKLNREDLLRKQRTFDNGSIPHQIHLGELHA
ILRRQEDFYPFLKDNREKIEKILTFRIPYYVGPLA
RGNSRFAWMTRKSEETITPWNFEEVVDKGASAQSF
IERMTNFDKNLPNEKVLPKHSLLYEYFTVYNELTK
VKYVTEGMRKPAFLSGEQKKAIVDLLFKTNRKVTV
KQLKEDYFKKIECFDSVEISGVEDRFNASLGTYHD
LLKIIKDKDFLDNEENEDILEDIVLTLTLFEDREM
IEERLKTYAHLFDDKVMKQLKRRRYTGWGRLSRKL
INGIRDKQSGKTILDFLKSDGFANRNFMQLIHDDS
LTFKEDIQKAQVSGQGDSLHEHIANLAGSPAIKKG
ILQTVKVVDELVKVMGRHKPENIVIEMARENQTTQ
KGQKNSRERMKRIEEGIKELGSQILKEHPVENTQL
QNEKLYLYYLQNGRDMYVDQELDINRLSDYDVDAI
VPQSFLKDDSIDNKVLTRSDKNRGKSDNVPSEEVV
KKMKNYWRQLLNAKLITQRKFDNLTKAERGGLSEL
DKAGFIKRQLVETRQITKHVAQILDSRMNTKYDEN
DKLIREVKVITLKSKLVSDFRKDFQFYKVREINNY
HHAHDAYLNAVVGTALIKKYPKLESEFVYGDYKVY
DVRKMIAKSEQEIGKATAKYFFYSNIMNFFKTEIT
LANGEIRKRPLIETNGETGEIVWDKGRDFATVRKV
LSMPQVNIVKKTEVQTGGFSKESILPKRNSDKLIA
RKKDWDPKKYGGFVSPTVAYSVLVVAKVEKGKSKK
LKSVKELLGITIMERSSFEKNPIDFLEAKGYKEVK
KDLIIKLPKYSLFELENGRKRMLASARELQKGNEL
ALPSKYVNFLYLASHYEKLKGSPEDNEQKQLFVEQ
HKHYLDEIIEQISEFSKRVILADANLDKVLSAYNK
HRDKPIREQAENIIHLFTLTNLGAPAAFKYFDTTI
DRKEYRSTKEVLDATLIHQSITGLYETRIDLSQLG
GD

In a particular embodiment, the Cas9 variant having expanded PAM capabilities is SpCas9 (H840A) VQR, having the following amino acid sequence (with the V, Q, R substitutions relative to the SpCas9 (H840A) sequence of SEQ ID NO: 107. In some embodiments, the disclosed base editors comprise a napDNAbp domain that comprises SpCas9-VQR.

VQR-nCas9 (D10A/D1135V/R1335Q/T1337R) S. pyogenes Cas9 Nickase

(SEQ ID NO: 156)
MDKKYSIGLAIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMIKRYDEHHQDLTLLKALVRQQLPEKYKEI
FFDQSKNGYAGYIDGGASQEEFYKFIKPILEKMDG
TEELLVKLNREDLLRKQRTFDNGSIPHQIHLGELH
AILRRQEDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRRRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGRHKPENIVIEMARENQTT
QKGQKNSRERMKRIEEGIKELGSQILKEHPVENTQ
LQNEKLYLYYLQNGRDMYVDQELDINRLSDYDVDH
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKRNSDKLI
ARKKDWDPKKYGGFVSPTVAYSVLVVAKVEKGKSK
KLKSVKELLGITIMERSSFEKNPIDFLEAKGYKEV
KKDLIIKLPKYSLFELENGRKRMLASAGELQKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGAPAAFKYFDTT
IDRKQYRSTKEVLDATLIHQSITGLYETRIDLSQL
GGD

In another particular embodiment, the Cas9 variant having expanded PAM capabilities is SpCas9 (H840A) EQR, having the D10A, D1135E, R1335Q, and T1337R substitutions relative to the SpCas9 (H840A) sequence of SEQ ID NO: 107. In addition, the methionine residue in SpCas9 (H840) was removed for SpCas9 (H840A) EQR):

(SEQ ID NO: 157)
MDKKYSIGLAIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMIKRYDEHHQDLTLLKALVRQQLPEKYKEI
FFDQSKNGYAGYIDGGASQEEFYKFIKPILEKMDG
TEELLVKLNREDLLRKQRTFDNGSIPHQIHLGELH
AILRRQEDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRRRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGRHKPENIVIEMARENQTT
QKGQKNSRERMKRIEEGIKELGSQILKEHPVENTQ
LQNEKLYLYYLQNGRDMYVDQELDINRLSDYDVDH
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKRNSDKLI
ARKKDWDPKKYGGFESPTVAYSVLVVAKVEKGKSK
KLKSVKELLGITIMERSSFEKNPIDFLEAKGYKEV
KKDLIIKLPKYSLFELENGRKRMLASAGELQKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGAPAAFKYFDTT
IDRKQYRSTKEVLDATLIHQSITGLYETRIDLSQL
GGD

In addition, any available methods may be utilized to obtain or construct a variant or mutant Cas9 protein. The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which are the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Because of their nature, gain-of-function mutations are usually dominant.

Mutations can be introduced into a reference Cas9 protein using site-directed mutagenesis. Older methods of site-directed mutagenesis known in the art rely on sub-cloning of the sequence to be mutated into a vector, such as an M13 bacteriophage vector, that allows the isolation of single-stranded DNA template. In these methods, one anneals a mutagenic primer (i.e., a primer capable of annealing to the site to be mutated but bearing one or more mismatched nucleotides at the site to be mutated) to the single-stranded template and then polymerizes the complement of the template starting from the 3′ end of the mutagenic primer. The resulting duplexes are then transformed into host bacteria and plaques are screened for the desired mutation. More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

Mutations may also be introduced by directed evolution processes, such as phage-assisted continuous evolution (PACE) or phage-assisted noncontinuous evolution (PANCE). The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference. Variant Cas9s may also be obtain by phage-assisted non-continuous evolution (PANCE), which as used herein, refers to non-continuous evolution that employs phage as viral vectors. PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Serial flask transfers have long served as a widely-accessible approach for laboratory evolution of microbes, and, more recently, analogous approaches have been developed for bacteriophage evolution. The PANCE system features lower stringency than the PACE system.

### (x) Cas9 Circular Permutants

In various embodiments, the base editors disclosed herein may comprise a circular permutant of Cas9.

The term “circularly permuted Cas9” or “circular permutant” of Cas9 or “CP-Cas9”) refers to any Cas9 protein, or variant thereof, that occurs or has been modify to engineered as a circular permutant variant, which means the N-terminus and the C-terminus of a Cas9 protein (e.g., a wild type Cas9 protein) have been topically rearranged. Such circularly permuted Cas9 proteins, or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, each of are incorporated herein by reference. Reference is also made to International Publication No. WO 2020/041751, published Feb. 27, 2020, herein incorporated by reference. The present disclosure contemplates any previously known CP-Cas9 or use a new CP-Cas9 so long as the resulting circularly permuted protein retains the ability to bind DNA when complexed with a guide RNA (gRNA).

Any of the Cas9 proteins described herein, including any variant, ortholog, or naturally occurring Cas9 or equivalent thereof, may be reconfigured as a circular permutant variant.

In various embodiments, the circular permutants of Cas9 may have the following structure: N-terminus-[original C-terminus]—[optional linker]—[original N-terminus]-C-terminus.

As an example, the present disclosure contemplates the following circular permutants of canonical S. pyogenes Cas9 (1368 amino acids of UniProtKB-Q99ZW2 (CAS9_STRP1) (numbering is based on the amino acid position in SEQ ID NO: 74)): N-terminus-[1268-1368]-[optional linker]-[1-1267]-C-terminus; N-terminus-[1168-1368]-[optional linker]-[1-1167]-C-terminus; N-terminus-[1068-1368]-[optional linker]-[1-1067]-C-terminus; N-terminus-[968-1368]-[optional linker]-[1-967]-C-terminus; N-terminus-[868-1368]-[optional linker]-[1-867]-C-terminus; N-terminus-[768-1368]-[optional linker]-[1-767]-C-terminus; N-terminus-[668-1368]-[optional linker]-[1-667]-C-terminus; N-terminus-[568-1368]-[optional linker]-[1-567]-C-terminus; N-terminus-[468-1368]-[optional linker]-[1-467]-C-terminus; N-terminus-[368-1368]-[optional linker]-[1-367]-C-terminus; N-terminus-[268-1368]-[optional linker]-[1-267]-C-terminus; N-terminus-[168-1368]-[optional linker]-[1-167]-C-terminus; N-terminus-[68-1368]-[optional linker]-[1-67]-C-terminus; or N-terminus-[10-1368]-[optional linker]-[1-9]-C-terminus, or the corresponding circular permutants of other Cas9 proteins (including other Cas9 orthologs, variants, etc).

In particular embodiments, the circular permuant Cas9 has the following structure (based on S. pyogenes Cas9 (1368 amino acids of UniProtKB-Q99ZW2 (CAS9_STRP1) (numbering is based on the amino acid position in SEQ ID NO: 74): N-terminus-[102-1368]-[optional linker]-[1-101]-C-terminus; N-terminus-[1028-1368]-[optional linker]-[1-1027]-C-terminus; N-terminus-[1041-1368]-[optional linker]-[1-1043]-C-terminus; N-terminus-[1249-1368]-[optional linker]-[1-1248]-C-terminus; or N-terminus-[1300-1368]-[optional linker]-[1-1299]-C-terminus, or the corresponding circular permutants of other Cas9 proteins (including other Cas9 orthologs, variants, etc).

In still other embodiments, the circular permuant Cas9 has the following structure (based on S. pyogenes Cas9 (1368 amino acids of UniProtKB-Q99ZW2 (CAS9_STRP1) (numbering is based on the amino acid position in SEQ ID NO: 74): N-terminus-[103-1368]-[optional linker]-[1-102]-C-terminus; N-terminus-[1029-1368]-[optional linker]-[1-1028]-C-terminus; N-terminus-[1042-1368]-[optional linker]-[1-1041]-C-terminus; N-terminus-[1250-1368]-[optional linker]-[1-1249]-C-terminus; or

N-terminus-[1301-1368]-[optional linker]-[1-1300]-C-terminus, or the corresponding circular permutants of other Cas9 proteins (including other Cas9 orthologs, variants, etc).

In some embodiments, the circular permutant can be formed by linking a C-terminal fragment of a Cas9 to an N-terminal fragment of a Cas9, either directly or by using a linker, such as an amino acid linker. In some embodiments, The C-terminal fragment may correspond to the C-terminal 95% or more of the amino acids of a Cas9 (e.g., amino acids about 1300-1368), or the C-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., any one of SEQ ID NOs: 18-25). The N-terminal portion may correspond to the N-terminal 95% or more of the amino acids of a Cas9 (e.g., amino acids about 1-1300), or the N-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., of SEQ ID NO: 74).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus, includes or corresponds to the C-terminal 30% or less of the amino acids of a Cas9 (e.g., amino acids 1012-1368 of SEQ ID NO: 74). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., the Cas9 of SEQ ID NO: 74). In some embodiments, the C-terminal fragment that is rearranged to the N-terminus, includes or corresponds to the C-terminal 410 residues or less of a Cas9 (e.g., the Cas9 of SEQ ID NO: 74). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g., the Cas9 of SEQ ID NO: 74). In some embodiments, the C-terminal portion that is rearranged to the N-terminus, includes or corresponds to the C-terminal 357, 341, 328, 120, or 69 residues of a Cas9 (e.g., the Cas9 of SEQ ID NO: 74).

In other embodiments, circular permutant Cas9 variants may be defined as a topological rearrangement of a Cas9 primary structure based on the following method, which is based on S. pyogenes Cas9 of SEQ ID NO: 74: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to preceed the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (relative the S. pyogenes Cas9 of SEQ ID NO: 74) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰, Cas9-CP³¹⁰, Cas9-CP¹⁰¹⁰, Cas9-CP¹⁰¹⁶, Cas9-CP¹⁰²³, Cas9-CP¹⁰²⁹, Cas9-CP¹⁰⁴¹, Cas9-CP^{1247, Cas}9-CP¹²⁴⁹, and Cas9-CP¹²⁸², respectively. This description is not meant to be limited to making CP variants from SEQ ID NO: 74, but may be implemented to make CP variants in any Cas9 sequence, either at CP sites that correspond to these positions, or at other CP sites entireley. This description is not meant to limit the specific CP sites in any way. Virtually any CP site may be used to form a CP-Cas9 variant.

Exemplary CP-Cas9 amino acid sequences, based on the Cas9 of SEQ ID NO: 74, are provided below in which linker sequences are indicated by underlining and optional methionine (M) residues are indicated in bold. It should be appreciated that the disclosure provides CP-Cas9 sequences that do not include a linker sequence or that include different linker sequences. It should be appreciated that CP-Cas9 sequences may be based on Cas9 sequences other than that of SEQ ID NO: 74 and any examples provided herein are not meant to be limiting. Exempalry CP-Cas9 sequences are as follows:

| CP name | Sequence | SEQ ID NO: |
|---|---|---|
| CP1012 | DYKVYDVRKMIAKSEQEIGKATAKYFFYSNIMNFFKTEITLANGEIRKRPLIETN | SEQ ID NO: |
| | GETGEIVWDKGRDFATVRKVLSMPQVNIVKKTEVQTGGFSKESILPKRNSDKLIA | 171 |
| | RKKDWDPKKYGGFDSPTVAYSVLVVAKVEKGKSKKLKSVKELLGITIMERSSEEK | |
| | NPIDFLEAKGYKEVKKDLIIKLPKYSLFELENGRKRMLASAGELQKGNELALPSK | |
| | YVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKE | |
| | VLDATLIHQSITGLYETRIDLSQLGGDGGSGGSGGSGGSGGSGGSGGDKKYSIGL | |
| | AIGTNSVGWAVITDEYKVPSKKFKVLGNTDRHSIKKNLIGALLFDSGETAEATRL | |
| | KRTARRRYTRRKNRICYLQEIFSNEMAKVDDSFFHRLEESFLVEEDKKHERHPIF | |
| | GNIVDEVAYHEKYPTIYHLRKKLVDSTDKADLRLIYLALAHMIKFRGHFLIEGDL | |
| | NPDNSDVDKLFIQLVQTYNQLFEENPINASGVDAKAILSARLSKSRRLENLIAQL | |
| | PGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALV | |
| | RQQLPEKYKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMTNFDKNLPN | |
| | EKVLPKHSLLYEYFTVYNELTKVKYVTEGMRKPAFLSGEQKKAIVDLLFKTNRKV | |
| | TVKQLKEDYFKKIECFDSVEISGVEDRFNASLGTYHDLLKIIKDKDFLDNEENED | |
| | ILEDIVLTLTLFEDREMIEERLKTYAHLFDDKVMKQLKRRRYTGWGRLSRKLING | |
| | IRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIEMARENQTTQKGQKNSRE | |
| | RMKRIEEGIKELGSQILKEHPVENTQLQNEKLYLYYLQNGRDMYVDQELDINRLS | |
| | DYDVDHIVPQSFLKDDSIDNKVLTRSDKNRGKSDNVPSEEVVKKMKNYWRQLLNA | |
| | KLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAWGTALIK | |
| | KYPKLESEFVYG | |
| CP1028 | EIGKATAKYFFYSNIMNFFKTEITLANGEIRKRPLIETNGETGEIVWDKGRDFAT | SEQ ID NO: |
| | VRKVLSMPQVNIVKKTEVQTGGFSKESILPKRNSDKLIARKKDWDPKKYGGFDSP | 172 |
| | TVAYSVLVVAKVEKGKSKKLKSVKELLGITIMERSSFEKNPIDFLEAKGYKEVKK | |
| | DLIIKLPKYSLFELENGRKRMLASAGELQKGNELALPSKYVNFLYLASHYEKLKG | |
| | SPEDNEQKQLFVEQHKHYLDEIIEQISEFSKRVILADANLDKVLSAYNKHRDKPI | |
| | REQAENIIHLFTLTNLGAPAAFKYFDTTIDRKRYTSTKEVLDATLIHQSITGLYE | |
| | TRIDLSQLGGDGGSGGSGGSGGSGGSGGSGGMDKKYSIGLAIGTNSVGWAVITDE | |
| | YKVPSKKFKVLGNTDRHSIKKNLIGALLFDSGETAEATRLKRTARRRYTRRKNRI | |
| | CYLQEIFSNEMAKVDDSFFHRLEESFLVEEDKKHERHPIFGNIVDEVAYHEKYPT | |
| | IYHLRKKLVDSTDKADLRLIYLALAHMIKFRGHFLIEGDLNPDNSDVDKLFIQLV | |
| | QTYNQLFEENPINASGVDAKAILSARLSKSRRLENLIAQLPGEKKNGLFGNLIAL | |
| | SLGLTPNFKSNFDLAEDAKLQLSKDTYDDDLDNLLAQIGDQYADLFLAAKNLSDA | |
| | ILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGS | |
| | IPHQIHLGELHAILRRQEDFYPFLKDNREKIEKILTFRIPYYVGPLARGNSRFAW | |
| | MTRKSEETITPWNFEEVVDKGASAQSFIERMTNFDKNLPNEKVLPKHSLLYEYFT | |
| | VYNELTKVKYVTEGMRKPAFLSGEQKKAIVDLLFKTNRKVTVKQLKEDYFKKIEC | |
| | FDSVEISGVEDRFNASLGTYHDLLKIIKDKDFLDNEENEDILEDIVLTLTLFEDR | |
| | EMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIKELGSQ | |
| | ILKEHPVENTQLQNEKLYLYYLQNGRDMYVDQELDINRLSDYDVDHIVPQSFLKD | |
| | DSIDNKVLTRSDKNRGKSDNVPSEEVVKKMKNYWRQLLNAKLITQRKFDNLTKAE | |
| | RGGLSELDKAGFIKRQLVETRQITKHVAQILDSRMNTKYDENDKLIREVKVITLK | |
| | SKLVSDFRKDFQFYKVREINNYHHAHDAYLNAVVGTALIKKYPKLESEFVYGDYK | |
| | VYDVRKMIAKSEQ | |
| CP1041 | NIMNFFKTEITLANGEIRKRPLIETNGETGEIVWDKGRDFATVRKVLSMPQVNIV | SEQ ID NO: |
| | KKTEVQTGGFSKESILPKRNSDKLIARKKDWDPKKYGGFDSPTVAYSVLVVAKVE | 173 |
| | KGKSKKLKSVKELLGITIMERSSFEKNPIDFLEAKGYKEVKKDLIIKLPKYSLFE | |
| | LENGRKRMLASAGELQKGNELALPSKYVNFLYLASHYEKLKGSPEDNEQKQLFVE | |
| | QHKHYLDEIIEQISEFSKRVILADANLDKVLSAYNKHRDKPIREQAENIIHLFTL | |
| | TNLGAPAAFKYFDTTIDRKRYTSTKEVLDATLIHQSITGLYETRIDLSQLGGDGG | |
| | SGGSGGSGGSGGSGGSGGDKKYSIGLAIGTNSVGWAVITDEYKVPSKKFKVLGNT | |
| | DRHSIKKNLIGALLFDSGETAEATRLKRTARRRYTRRKNRICYLQEIFSNEMAKV | |
| | DDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINA | |
| | SGVDAKAILSARLSKSRRLENLIAQLPGEKKNGLFGNLIALSLGLTPNFKSNFDL | |
| | AEDAKLQLSKDTYDDDLDNLLAQIGDQYADLFLAAKNLSDAILLSDILRVNTEIT | |
| | KAPLSASMIKRYDEHHQDLTLLKALVRQQLPEKYKEIFFDQSKNGYAGYIDGGAS | |
| | QEEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPWNF | |
| | EEVVDKGASAQSFIERMTNFDKNLPNEKVLPKHSLLYEYFTVYNELTKVKYVTEG | |
| | MRKPAFLSGEQKKAIVDLLFKTNRKVTVKQLKEDYFKKIECFDSVEISGVEDRFN | |
| | ASLGTYHDLLKIIKDKDFLDNEENEDILEDIVLTLTLFEDREMIEERLKTYAHLF | |
| | DDKVMKQLKRRRYTGWGRLSRKLINGIRDKQSGKTILDFLKSDGFANRNFMQLIH | |
| | DDSLTFKEDIQKAQVSGQGDSLHEHIANLAGSPAIKKGILQTVKVVDELVKVMGR | |
| | HKPENIVIEMARENQTTQKGQKNSRERMKRIEEGIKELGSQILKEHPVENTQLQN | |
| | EKLYLYYLQNGRDMYVDQELDINRLSDYDVDHIVPQSFLKDDSIDNKVLTRSDKN | |
| | RGKSDNVPSEEVVKKMKNYWRQLLNAKLITQRKFDNLTKAERGGLSELDKAGFIK | |
| | RQLVETRQITKHVAQILDSRMNTKYDENDKLIREVKVITLKSKLVSDFRKDFQFY | |
| | KVREINNYHHAHDAYLNAVVGTALIKKYPKLESEFVYGDYKVYDVRKMIAKSEQE | |
| | IGKATAKYFFYS | |
| CP1249 | PEDNEQKQLFVEQHKHYLDEIIEQISEFSKRVILADANLDKVLSAYNKHRDKPIR | SEQ ID NO: |
| | EQAENIIHLFTLTNLGAPAAFKYFDTTIDRKRYTSTKEVLDATLIHQSITGLYET | 174 |
| | RIDLSQLGGDGGSGGSGGSGGSGGSGGSGGMDKKYSIGLAIGTNSVGWAVITDEY | |
| | KVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTI | |
| | YHLRKKLVDSTDKADLRLIYLALAHMIKFRGHFLIEGDLNPDNSDVDKLFIQLVQ | |
| | TYNQLFEENPINASGVDAKAILSARLSKSRRLENLIAQLPGEKKNGLFGNLIALS | |
| | LGLTPNFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEIFFDQS | |
| | KNGYAGYIDGGASQEEFYKFIKPILEKMDGTEELLVKLNREDLLRKQRTFDNGSI | |
| | PHQIHLGELHAILRRQEDFYPFLKDNREKIEKILTFRIPYYVGPLARGNSRFAWM | |
| | TRKSEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKIECF | |
| | DSVEISGVEDRFNASLGTYHDLLKIIKDKDFLDNEENEDILEDIVLTLTLFEDRE | |
| | MIEERLKTYAHLFDDKVMKQLKRRRYTGWGRLSRKLINGIRDKQSGKTILDFLKS | |
| | DGFANRNFMQLIHDDSLTFKEDIQKAQVSGQGDSLHEHIANLAGSPAIKKGILQT | |
| | VKVVDELVKVMGRHKPENIVIEMARENQTTQKGQKNSRERMKRIEEGIKELGSQI | |
| | LKEHPVENTQLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRKFDNLTKAER | |
| | GGLSELDKAGFIKRQLVETRQITKHVAQILDSRMNTKYDENDKLIREVKVITLKS | |
| | KLVSDFRKDFQFYKVREINNYHHAHDAYLNAVVGTALIKKYPKLESEFVYGDYKV | |
| | YDVRKMIAKSEQEIGKATAKYFFYSNIMNFFKTEITLANGEIRKRPLIETNGETG | |
| | EIVWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNF | |
| | LYLASHYEKLKGS | |
| CP1300 | KPIREQAENIIHLFTLTNLGAPAAFKYFDTTIDRKRYTSTKEVLDATLIHQSITG | SEQ ID NO: |
| | LYETRIDLSQLGGDGGSGGSGGSGGSGGSGGSGGDKKYSIGLAIGTNSVGWAVIT | 175 |
| | DEYKVPSKKFKVLGNTDRHSIKKNLIGALLFDSGETAEATRLKRTARRRYTRRKN | |
| | RICYLQEIFSNEMAKVDDSFFHRLEESFLVEEDKKHERHPIFGNIVDEVAYHEKY | |
| | PTIYHLRKKLVDSTDKADLRLIYLALAHMIKFRGHFLIEGDLNPDNSDVDKLFIQ | |
| | LVQTYNQLFEENPINASGVDAKAILSARLSKSRRLENLIAQLPGEKKNGLFGNLI | |
| | ALSLGLTPNFKSNFDLAEDAKLQLSKDTYDDDLDNLLAQIGDQYADLFLAAKNLS | |
| | DAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDN | |
| | GSIPHQIHLGELHAILRRQEDFYPFLKDNREKIEKILTFRIPYYVGPLARGNSRF | |
| | AWMTRKSEETITPWNFEEVVDKGASAQSFIERMTNFDKNLPNEKVLPKHSLLYEY | |
| | FTVYNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGI | |
| | LQTVKVVDELVKVMGRHKPENIVIEMARENQTTQKGQKNSRERMKRIEEGIKELG | |
| | SQILKEHPVENTQLQNEKLYLYYLQNGRDMYVDQELDINRLSDYDVDHIVPQSFL | |
| | KDDSIDNKVLTRSDKNRGKSDNVPSEEVVKKMKNYWRQLLNAKLITQRKFDNLTK | |
| | AERGGLSELDKAGFIKRQLVETRQITKHVAQILDSRMNTKYDENDKLIREVKVIT | |
| | LKSKLVSDFRKDFQFYKVREINNYHHAHDAYLNAVVGTALIKKYPKLESEFVYGD | |
| | YKVYDVRKMIAKSEQEIGKATAKYFFYSNIMNFFKTEITLANGEIRKRPLIETNG | |
| | ETGEIVWDKGRDFATVRKVLSMPQVNIVKKTEVQTGGFSKESILPKRNSDKLIAR | |
| | KKDWDPKKYGGFDSPTVAYSVLVVAKVEKGKSKKLKSVKELLGITIMERSSFEKN | |
| | PIDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADANL | |
| | DKVLSAYNKHRD | |

The Cas9 circular permutants that may be useful in the base editing constructs described herein. Exemplary C-terminal fragments of Cas9, based on the Cas9 of SEQ ID NO: 74, which may be rearranged to an N-terminus of Cas9, are provided below. It should be appreciated that such C-terminal fragments of Cas9 are exemplary and are not meant to be limiting. These exemplary CP-Cas9 fragments have the following sequences:

| CP name | Sequence | SEQ ID NO: |
|---|---|---|
| CP1012 c- | DYKVYDVRKMIAKSEQEIGKATAKY | SEQ ID NO: |
| terminal | FFYSNIMNFFKTEITLANGEIRKRP | 176 |
| fragment | LIETNGETGEIVWDKGRDFATVRKV | |
| | LSMPQVNIVKKTEVQTGGFSKESIL | |
| | PKRNSDKLIARKKDWDPKKYGGFDS | |
| | PTVAYSVLVVAKVEKGKSKKLKSVK | |
| | ELLGITIMERSSFEKNPIDFLEAKG | |
| | YKEVKKDLIIKLPKYSLFELENGRK | |
| | RMLASAGELQKGNELALPSKYVNFL | |
| | YLASHYEKLKGSPEDNEQKQLFVEQ | |
| | HKHYLDEIIEQISEFSKRVILADAN | |
| | LDKVLSAYNKHRDKPIREQAENIIH | |
| | LFTLTNLGAPAAFKYFDTTIDRKRY | |
| | TSTKEVLDATLIHQSITGLYETRID | |
| | LSQLGGD | |
| CP1028 c- | EIGKATAKYFFYSNIMNFFKTEITL | SEQ ID NO: |
| terminal | ANGEIRKRPLIETNGETGEIVWDKG | 177 |
| fragment | RDFATVRKVLSMPQVNIVKKTEVQT | |
| | GGFSKESILPKRNSDKLIARKKDWD | |
| | PKKYGGFDSPTVAYSVLVVAKVEKG | |
| | KSKKLKSVKELLGITIMERSSFEKN | |
| | PIDFLEAKGYKEVKKDLIIKLPKYS | |
| | LFELENGRKRMLASAGELQKGNELA | |
| | LPSKYVNFLYLASHYEKLKGSPEDN | |
| | EQKQLFVEQHKHYLDEIIEQISEFS | |
| | KRVILADANLDKVLSAYNKHRDKPI | |
| | REQAENIIHLFTLTNLGAPAAFKYF | |
| | DTTIDRKRYTSTKEVLDATLIHQSI | |
| | TGLYETRIDLSQLGGD | |
| CP1041 c- | NIMNFFKTEITLANGEIRKRPLIET | SEQ ID NO: |
| terminal | NGETGEIVWDKGRDFATVRKVLSMP | 178 |
| fragment | QVNIVKKTEVQTGGFSKESILPKRN | |
| | SDKLIARKKDWDPKKYGGFDSPTVA | |
| | YSVLWAKVEKGKSKKLKSVKELLGI | |
| | TIMERSSFEKNPIDFLEAKGYKEVK | |
| | KDLIIKLPKYSLFELENGRKRMLAS | |
| | AGELQKGNELALPSKYVNFLYLASH | |
| | YEKLKGSPEDNEQKQLFVEQHKHYL | |
| | DEIIEQISEFSKRVILADANLDKVL | |
| | SAYNKHRDKPIREQAENIIHLFTLT | |
| | NLGAPAAFKYFDTTIDRKRYTSTKE | |
| | VLDATLIHQSITGLYETRIDLSQLG | |
| | GD | |
| CP1249 c- | PEDNEQKQLFVEQHKHYLDEIIEQI | SEQ ID NO: |
| terminal | SEFSKRVILADANLDKVLSAYNKHR | 179 |
| fragment | DKPIREQAENIIHLFTLTNLGAPAA | |
| | FKYFDTTIDRKRYTSTKEVLDATLI | |
| | HQSITGLYETRIDLSQLGGD | |
| CP1300 c- | KPIREQAENIIHLFTLTNLGAPAAF | SEQ ID NO: |
| terminal | KYFDTTIDRKRYTSTKEVLDATLIH | 180 |
| fragment | QSITGLYETRIDLSQLGGD | |

In some embodiments, the napDNAbp domain comprises a combination of more than one Cas homolog or variant, such as a circularly permuted Cas variant. In some embodiments, the napDNAbp domain comprises a first Cas variant and a second Cas variant. In some embodiments, the napDNAbp domain comprises a first Cas variant comprising a Cas9-NG and a second Cas variant comprising a Cas9-CP1041 variant. The combination of the CP1041 variant and the NG variant enables both broadened PAM targeting and an expanded editing window. Such a domain is referred to herein as “SpCas9-NG-CP1041.” In some embodiments, the napDNAbp domain comprises an amino acid sequence that has at least 80%, at least 8%, at least 90%, at least 92.5%, at least 95%, at least 97.5%, at least 98%, or at least 99% sequence identity to SEQ ID NO: 181. In some embodiments, the napDNAbp domain comprises the sequence of SEQ ID NO: 181

(SEQ ID NO: 181)
NIMNFFKTEITLANGEIRKRPLIETNGETGEIVWD
KGRDFATVRKVLSMPQVNIVKKTEVQTGGFSKESI
RPKRNSDKLIARKKDWDPKKYGGFVSPTVAYSVLV
VAKVEKGKSKKLKSVKELLGITIMERSSFEKNPID
FLEAKGYKEVKKDLIIKLPKYSLFELENGRKRMLA
SARFLQKGNELALPSKYVNFLYLASHYEKLKGSPE
DNEQKQLFVEQHKHYLDEIIEQISEFSKRVILADA
NLDKVLSAYNKHRDKPIREQAENIIHLFTLTNLGA
PRAFKYFDTTIDRKVYRSTKEVLDATLIHQSITGL
YETRIDLSQLGGDGGSGGSGGSGGSGGSGGSGGDK
KYSIGLAIGTNSVGWAVITDEYKVPSKKFKVLGNT
DRHSIKKNLIGALLFDSGETAEATRLKRTARRRYT
RRKNRICYLQEIFSNEMAKVDDSFFHRLEESFLVE
EDKKHERHPIFGNIVDEVAYHEKYPTIYHLRKKLV
DSTDKADLRLIYLALAHMIKFRGHFLIEGDLNPDN
SDVDKLFIQLVQTYNQLFEENPINASGVDAKAILS
ARLSKSRRLENLIAQLPGEKKNGLFGNLIALSLGL
TPNFKSNFDLAEDAKLQLSKDTYDDDLDNLLAQIG
DQYADLFLAAKNLSDAILLSDILRVNTEITKAPLS
ASMIKRYDEHHQDLTLLKALVRQQLPEKYKEIFFD
QSKNGYAGYIDGGASQEEFYKFIKPILEKMDGTEE
LLVKLNREDLLRKQRTFDNGSIPHQIHLGELHAIL
RRQEDFYPFLKDNREKIEKILTFRIPYYVGPLARG
NSRFAWMTRKSEETITPWNFEEVVDKGASAQSFIE
RMTNFDKNLPNEKVLPKHSLLYEYFTVYNELTKVK
YVTEGMRKPAFLSGEQKKAIVDLLFKTNRKVTVKQ
LKEDYFKKIECFDSVEISGVEDRFNASLGTYHDLL
KIIKDKDFLDNEENEDILEDIVLTLTLFEDREMIE
ERLKTYAHLFDDKVMKQLKRRRYTGWGRLSRKLIN
GIRDKQSGKTILDFLKSDGFANRNFMQLIHDDSLT
FKEDIQKAQVSGQGDSLHEHIANLAGSPAIKKGIL
QTVKVVDELVKVMGRHKPENIVIEMARENQTTQKG
QKNSRERMKRIEEGIKELGSQILKEHPVENTQLQN
EKLYLYYLQNGRDMYVDQELDINRLSDYDVDHIVP
QSFLKDDSIDNKVLTRSDKNRGKSDNVPSEEVVKK
MKNYWRQLLNAKLITQRKFDNLTKAERGGLSELDK
AGFIKRQLVETRQITKHVAQILDSRMNTKYDENDK
LIREVKVITLKSKLVSDFRKDFQFYKVREINNYHH
AHDAYLNAVVGTALIKKYPKLESEFVYGDYKVYDV
RKMIAKSEQEIGKATAKYFFYS

In some embodiments, the napDNAbp domain comprises a first Cas variant comprising a Cas9-VRQR and a second Cas variant comprising a Cas9-CP1041 variant. Such a domain is referred to herein as “SpCas9-NG-VRQR.” In some embodiments, the napDNAbp domain comprises an amino acid sequence that has at least 80%, at least 8%, at least 90%, at least 92.5%, at least 95%, at least 97.5%, at least 98%, or at least 99% sequence identity to SEQ ID NO: 182. In some embodiments, the napDNAbp domain comprises the sequence of SEQ ID NO: 182.

(SEQ ID NO: 182)
NIMNFFKTEITLANGEIRKRPLIETNGETGEIVWD
KGRDFATVRKVLSMPQVNIVKKTEVQTGGFSKESI
RPKRNSDKLIARKKDWDPKKYGGFVSPTVAYSVLV
VAKVEKGKSKKLKSVKELLGITIMERSSFEKNPID
FLEAKGYKEVKKDLIIKLPKYSLFELENGRKRMLA
SARFLQKGNELALPSKYVNFLYLASHYEKLKGSPE
DNEQKQLFVEQHKHYLDEIIEQISEFSKRVILADA
NLDKVLSAYNKHRDKPIREQAENIIHLFTLTNLGA
PRAFKYFDTTIDRKVYRSTKEVLDATLIHQSITGL
YETRIDLSQLGGDGGSGGSGGSGGSGGSGGSGGDK
KYSIGLAIGTNSVGWAVITDEYKVPSKKFKVLGNT
DRHSIKKNLIGALLFDSGETAEATRLKRTARRRYT
RRKNRICYLQEIFSNEMAKVDDSFFHRLEESFLVE
EDKKHERHPIFGNIVDEVAYHEKYPTIYHLRKKLV
DSTDKADLRLIYLALAHMIKFRGHFLIEGDLNPDN
SDVDKLFIQLVQTYNQLFEENPINASGVDAKAILS
ARLSKSRRLENLIAQLPGEKKNGLFGNLIALSLGL
TPNFKSNFDLAEDAKLQLSKDTYDDDLDNLLAQIG
DQYADLFLAAKNLSDAILLSDILRVNTEITKAPLS
ASMIKRYDEHHQDLTLLKALVRQQLPEKYKEIFFD
QSKNGYAGYIDGGASQEEFYKFIKPILEKMDGTEE
LLVKLNREDLLRKQRTFDNGSIPHQIHLGELHAIL
RRQEDFYPFLKDNREKIEKILTFRIPYYVGPLARG
NSRFAWMTRKSEETITPWNFEEVVDKGASAQSFIE
RMTNFDKNLPNEKVLPKHSLLYEYFTVYNELTKVK
YVTEGMRKPAFLSGEQKKAIVDLLFKTNRKVTVKQ
LKEDYFKKIECFDSVEISGVEDRFNASLGTYHDLL
KIIKDKDFLDNEENEDILEDIVLTLTLFEDREMIE
ERLKTYAHLFDDKVMKQLKRRRYTGWGRLSRKLIN
GIRDKQSGKTILDFLKSDGFANRNFMQLIHDDSLT
FKEDIQKAQVSGQGDSLHEHIANLAGSPAIKKGIL
QTVKVVDELVKVMGRHKPENIVIEMARENQTTQKG
QKNSRERMKRIEEGIKELGSQILKEHPVENTQLQN
EKLYLYYLQNGRDMYVDQELDINRLSDYDVDHIVP
QSFLKDDSIDNKVLTRSDKNRGKSDNVPSEEVVKK
MKNYWRQLLNAKLITQRKFDNLTKAERGGLSELDK
AGFIKRQLVETRQITKHVAQILDSRMNTKYDENDK
LIREVKVITLKSKLVSDFRKDFQFYKVREINNYHH
AHDAYLNAVVGTALIKKYPKLESEFVYGDYKVYDV
RKMIAKSEQEIGKATAKYFFYS

Any of the references noted above which relate to Cas9 or Cas9 equivalents are hereby incorporated by reference in their entireties, if not already stated so.

### Adenosine Deaminase Domains

The disclosure provides base editors that comprise an adenosine deaminase domain. In some aspects, any of the disclosed base editors are capable of deaminating adenosine in a nucleic acid sequence (e.g., DNA). As one example, any of the base editors described herein may be base editors (e.g., adenine base editors). In some embodiments, the disclosed adenosine deaminases comprise known adenosine deaminase TadA7.10, or comprise variants of TadA7.10, which comprises the following mutations as compared to wild-type ecTadA (SEQ ID NO: 325): W23R, H36L, P48A, R51L, L84F, A106V, D108N, H123Y, S146C, D147Y, R152P, E155V, I156F, and K157N. In some embodiments, the adenosine deaminase comprises A109S, T111R, D119N, H122N, Y147D, F149Y, T1661, and D167N mutations in TadA7.10. This adenosine deaminase variant is referred to herein as TadA-8e, or TadA8.

In various embodiments, the adenosine deaminases of the disclosed base editors hydrolytically deaminate a targeted adenosine in a nucleic acid of interest to an inosine, which is read as a guanosine (G) by DNA polymerase enzymes.

In some embodiments, the adenosine deaminase domain of any of the disclosed base editors comprises a single adenosine deaminase, or a monomer. In some embodiments, the adenosine deaminase domain comprises 2, 3, 4 or 5 adenosine deaminases. In some embodiments, the adenosine deaminase domain comprises two adenosine deaminases, or a dimer. In some embodiments, the deaminase domain comprises a dimer of an engineered (or evolved) deaminase and a wild-type deaminase, such as a wild-type E. coli deaminase. One of skill in the art will be able to identify the corresponding residue in any homologous protein and in the respective encoding nucleic acid by methods well known in the art, e.g., by sequence alignment and determination of homologous residues. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is from a prokaryote. In some embodiments, the adenosine deaminase is from a bacterium. In some embodiments, the adenosine deaminase is from Escherichia coli, Staphylococcus aureus, Streptococcus pyogenes, Salmonella typhi, Shewanella putrefaciens, Haemophilus influenzae, Caulobacter crescentus, or Bacillus subtilis. In some embodiments, the adenosine deaminase is from E. coli. It should be appreciated that the mutations provided herein (e.g., mutations in ecTadA) may be applied to adenosine deaminases in other adenosine base editors, for example, those provided in International Publication No. WO 2018/027078, published Aug. 2, 2018; International Publication No. WO 2019/079347 on Apr. 25, 2019; International Application No PCT/US2019/033848, filed May 23, 2019, which published as International Publication No. WO 2019/226593 on Nov. 28, 2019; U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, and U.S. Provisional Application No. 62/835,490, filed Apr. 17, 2019, International Publication No. WO 2020/214842, published Oct. 22, 2020, International Publication No. WO2020/236982, published Nov. 26, 2020. and International Application No. PCT/US2021/016827, filed Feb. 5, 2021; all of which are incorporated herein by reference in their entireties.

Additional exemplary adenosine deaminases for use in the editing methods provided herein include TadA mutants TadA*8.1, TadA*8.2, TadA*8.3, TadA*8.4, TadA*8.5, TadA*8.6, TadA*8.7, TadA*8.8, TadA*8.9, TadA*8 10, TadA*8.11, TadA*8.12, TadA*8.13, TadA*8 14, TadA*8.15, TadA*8.16, TadA*8.17, TadA*8 18, TadA*8 19, TadA*8.20, TadA*8.21, TadA*8 22, TadA*8.23, and TadA*8.24. See WO 2020/231863, published Nov. 19, 2020, incorporated herein by reference.

In some embodiments, the adenosine deaminase is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring adenosine deaminase. In some embodiments, the adenosine deaminase is from a bacterium, such as, E.coli, S. aureus, S. pyogenes S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5 ,6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine

In some embodiments, the TadA deaminase is an N-terminal truncated TadA. In certain embodiments, the adenosine deaminase comprises the amino acid sequence:

(SEQ ID NO: 278)
MSEVEFSHEYWMRHALTLAKRAWDEREVPVGAVLV
HNNRVIGEGWNRPIGRHDPTAHAEIMALRQGGLVM
QNYRLIDATLYVTLEPCVMCAGAMIHSRIGRVVFG
ARDAKTGAAGSLMDVLHHPGMNHRVEITEGILADE
CAALLSDFFRMRRQEIKAQKKAQSSTD.

In some embodiments the TadA deaminase is a full-length E. coli TadA deaminase. For example, in certain embodiments, the adenosine deaminase comprises the amino acid sequence:

(SEQ ID NO: 279)
MRRAFITGVFFLSEVEFSHEYWMRHALTLAKRAWD
EREVPVGAVLVHNNRVIGEGWNRPIGRHDPTAHAE
IMALRQGGLVMQNYRLIDATLYVTLEPCVMCAGAM
IHSRIGRWFGARDAKTGAAGSLMDVLHHPGMNHRV
EITEGILADECAALLSDFFRMRRQEIKAQKKAQSS
TD

It should be appreciated, however, that additional adenosine deaminases useful in the present application would be apparent to the skilled artisan and are within the scope of this disclosure. For example, the adenosine deaminase may be a homolog of an ADAT. Exemplary ADAT homologs include, without limitation:

Staphylococcus aureus
TadA:
(SEQ ID NO: 280)
MGSHMTNDIYFMTLAIEEAKKAAQLGEVPIGAIIT
KDDEVIARAHNLRETLQQPTAHAEHIAIERAAKVL
GSWRLEGCTLYVTLEPCVMCAGTIVMSRIPRVVYG
ADDPKGGCSGSLMNLLQQSNFNHRAIVDKGVLKEA
CSTLLTTFFKNLRANKKSTN
Bacillus subtilis
TadA:
(SEQ ID NO: 281)
MTQDELYMKEAIKEAKKAEEKGEVPIGAVLVINGE
IIARAHNLRETEQRSIAHAEMLVIDEACKALGTWR
LEGATLYVTLEPCPMCAGAVVLSRVEKVVFGAFDP
KGGCSGTLMNLLQEERFNHQAEVVSGVLEEECGGM
LSAFFRELRKKKKAARKNLSE
Salmonella typhimurium (S. typhimurium)
TadA:
(SEQ ID NO: 282)
MPPAFITGVTSLSDVELDHEYWMRHALTLAKRAWD
EREVPVGAVLVHNHRVIGEGWNRPIGRHDPTAHAE
IMALRQGGLVLQNYRLLDTTLYVTLEPCVMCAGAM
VHSRIGRVVFGARDAKTGAAGSLIDVLHHPGMNHR
VEIIEGVLRDECATLLSDFFRMRRQEIKALKKADR
AEGAGPAV
Shewanella putrefaciens (S. putrefaciens)
TadA:
(SEQ ID NO: 283)
MDEYWMQVAMQMAEKAEAAGEVPVGAVLVKDGQQI
ATGYNLSISQHDPTAHAEILCLRSAGKKLENYRLL
DATLYITLEPCAMCAGAMVHSRIARVVYGARDEKT
GAAGTVVNLLQHPAFNHQVEVTSGVLAEACSAQLS
RFFKRRRDEKKALKLAQRAQQGIE
Haemophilus influenzae F3031 (H. influenzae)
TadA:
(SEQ ID NO: 284)
MDAAKVRSEFDEKMMRYALELADKAEALGEIPVGA
VLVDDARNIIGEGWNLSIVQSDPTAHAEIIALRNG
AKNIQNYRLLNSTLYVTLEPCTMCAGAILHSRIKR
LVFGASDYKTGAIGSRFHFFDDYKMNHTLEITSGV
LAEECSQKLSTFFQKRREEKKIEKALLKSLSDK
Caulobacter crescentus (C. crescentus)
TadA:
(SEQ ID NO: 285)
MRTDESEDQDHRMMRLALDAARAAAEAGETPVGAV
ILDPSTGEVIATAGNGPIAAHDPTAHAEIAAMRAA
AAKLGNYRLTDLTLVVTLEPCAMCAGAISHARIGR
VVFGADDPKGGAVVHGPKFFAQPTCHWRPEVTGGV
LADESADLLRGFFRARRKAKI
Geobacter sulfurreducens (G. sulfurreducens)
TadA:
(SEQ ID NO: 286)
MSSLKKTPIRDDAYWMGKAIREAAKAAARDEVPIG
AVIVRDGAVIGRGHNLREGSNDPSAHAEMIAIRQA
ARRSANWRLTGATLYVTLEPCLMCMGAIILARLER
VVFGCYDPKGGAAGSLYDLSADPRLNHQVRLSPGV
CQEECGTMLSDFFRDLRRRKKAKATPALFIDERKV
PPEP
Streptococcus pyogenes (S. pyogenes) TadA
(SEQ ID NO: 287)
MPYSLEEQTYFMQEALKEAEKSLQKAEIPIGCVIV
KDGEIIGRGHNAREESNQAIMHAEIMAINEANAHE
GNWRLLDTTLFVTIEPCVMCSGAIGLARIPHVIYG
ASNQKFGGADSLYQILTDERLNHRVQVERGLLAAD
CANIMQTFFRQGRERKKIAKHLIKEQSDPFD

Exemplary TadA deaminase domains of the presently disclosed base editors include, without limitation, any of the following adenosein deaminases:

TadA7.10:
(SEQ ID NO: 288)
SEVEFSHEYWMRHALTLAKRALDEREVPVGAVLVL
NNRVIGEGWNRAIGLHDPTAHAEIMALRQGGLVMQ
NYRLIDATLYVTFEPCVMCAGAMIHSRIGRVVFGV
RNAKTGAAGSLMDVLHYPGMNHRVEITEGILADEC
AALLCYFFRMPRQVFNAQKKAQSSTD
TadA 7.10 (V106W) (E. coli)
(SEQ ID NO: 289)
SEVEFSHEYWMRHALTLAK
RARDEREVPVGAVLVLNNRVIGEGWNRAIGLHDPT
AHAEIMALRQGGLVMQNYRLIDATLYVTFEPCVMC
AGAMIHSRIGRVVFGWRNAKTGAAGSLMDVLHYPG
MNHRVEITEGILADECAALLCYFFRMPRQVFNAQK
KAQSSTD
TadA-8e (E. coli)
(SEQ ID NO: 290)
SEVEFSHEYWMRHALTLAKRARDEREVPVGAVLVL
NNRVIGEGWNRAIGLHDPTAHAEIMALRQGGLVMQ
NYRLIDATLYVTFEPCVMCAGAMIHSRIGRVVFGV
RNSKRGAAGSLMNVLNYPGMNHRVEITEGILADEC
AALLCDFYRMPRQVFNAQKKAQSSIN
TadA-8e(V106W) (E. coli)
(SEQ ID NO: 291)
SEVEFSHEYWMRHALTLAKRARDEREVPVGAVLVL
NNRVIGEGWNRAIGLHDPTAHAEIMALRQGGLVMQ
NYRLIDATLYVTFEPCVMCAGAMIHSRIGRVVFGW
RNSKRGAAGSLMNVLNYPGMNHRVEITEGILADEC
AALLCDFYRMPRQVFNAQKKAQSSIN

In some embodiments, the TadA 7.10 of SEQ ID NO: 2780 comprises an N-terminal methionine. It should be appreciated that the amino acid numbering scheme relating to the mutations in TadA 7.10 may be based on the TadA sequence of SEQ ID NO: 278, which contains an N-terminal methionine.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 278-291, or to any of the adenosine deaminases described herein. It should be appreciated that adenosine deaminases described herein may include one or more mutations (e.g., any of the mutations provided herein). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acids that differ relative to any one of the amino acid sequences set forth in SEQ ID NOs: 278-291, or any of the adenosine deaminases described herein. These differences may comprise amino acids that have been inserted, deleted, or substituted relative to the amino acid sequence of any of SEQ ID NOs: 278-291. In some embodiments, any of the adenosine deaminase domains described herein may comprise an amino acid sequence having 1, 2, 3, 4, 5, 6, 7, 8, 9, 10, 11, 12, 13, 14, 15, 16, 17, 18, 19, 20, 25, 30, or more than 30 amino acids that differ relative to the amino acid sequence of a wild type adenosine deaminase (e.g., ecTadA). In some embodiments, the adenosine deaminase domains comprise truncations at the N-terminus or C-terminus relative to a wild-type adenosine deaminase.

In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences set forth in SEQ ID NOs: 278-291, or any of the adenosine deaminases described herein.

In some embodiments, the adenosine deaminase comprises a D108X mutation in ecTadA SEQ ID NO: 278, or a corresponding mutation in another adenosine deaminase (e.g., SEQ ID NO: 279-291), where X indicates any amino acid other than the corresponding amino acid in the wild-type adenosine deaminase. In some embodiments, the adenosine deaminase comprises a D108G, D108N, D108V, D108A, or D108Y mutation in SEQ ID NO: 278, or a corresponding mutation in another adenosine deaminase (e.g., SEQ ID NO: 279-291). In some embodiments, the adenosine deaminase comprises a D108N mutation in SEQ ID NO: 278, or a corresponding mutation in another adenosine deaminase (e.g., SEQ ID NO: 279-291). It should be appreciated, however, that additional deaminases may similarly be aligned to identify homologous amino acid residues that can be mutated as provided herein.

In some embodiments, the adenosine deaminase comprises an A106X mutation in ecTadA SEQ ID NO: 278, or a corresponding mutation in another adenosine deaminase (e.g., SEQ ID NO: 279-291), where X indicates any amino acid other than the corresponding amino acid in the wild-type adenosine deaminase. In some embodiments, the adenosine deaminase comprises an A106V mutation in SEQ ID NO: 278, or a corresponding mutation in another adenosine deaminase (e.g., SEQ ID NO: 279-291).

In some embodiments, the adenosine deaminase comprises a E155X mutation in SEQ ID NO: 278, or a corresponding mutation in another adenosine deaminase (e.g., SEQ ID NO: 279-291), where the presence of X indicates any amino acid other than the corresponding amino acid in the wild-type adenosine deaminase. In some embodiments, the adenosine deaminase comprises a E155D, E155G, or E155V mutation in SEQ ID NO: 278, or a corresponding mutation in another adenosine deaminase (e.g., SEQ ID NO: 279-291). In some embodiments, the adenosine deaminase comprises a E155V mutation in SEQ ID NO: 278, or a corresponding mutation in another adenosine deaminase (e.g., SEQ ID NO: 279-291).

In some embodiments, the adenosine deaminase comprises a D147X mutation in SEQ ID NO: 278, or a corresponding mutation in another adenosine deaminase (e.g., SEQ ID NO: 279-291), where the presence of X indicates any amino acid other than the corresponding amino acid in the wild-type adenosine deaminase. In some embodiments, the adenosine deaminase comprises a D147Y mutation in SEQ ID NO: 278, or a corresponding mutation in another adenosine deaminase (e.g., SEQ ID NO: 279-291).

It should be appreciated that any of the mutations provided herein (e.g., based on the ecTadA amino acid sequence of SEQ ID NO: 278) may be introduced into other adenosine deaminases, such as S. aureus TadA (saTadA), or other adenosine deaminases (e.g., bacterial adenosine deaminases). It would be apparent to the skilled artisan how to identify amino acid residues from other adenosine deaminases that are homologous to the mutated residues in ecTadA. Thus, any of the mutations identified in ecTadA may be made in other adenosine deaminases that have homologous amino acid residues. It should also be appreciated that any of the mutations provided herein may be made individually or in any combination in ecTadA or another adenosine deaminase (e.g., SEQ ID NO: 279-291). For example, an adenosine deaminase may contain a D108N, a A106V, a E155V, and/or a D147Y mutation in ecTadA SEQ ID NO: 278, or a corresponding mutation in another adenosine deaminase (e.g., SEQ ID NO: 279-291). In some embodiments, an adenosine deaminase comprises the following group of mutations (groups of mutations are separated by a “;”) in ecTadA SEQ ID NO: 278, or corresponding mutations in another adenosine deaminase (e.g., SEQ ID NO: 279-291): D108N and A106V; D108N and E155V; D108N and D147Y; A106V and E155V; A106V and D147Y; E155V and D147Y; D108N, A106V, and E55V; D108N, A106V, and D147Y; D108N, E55V, and D147Y; A106V, E55V, and D147Y; and D108N, A106V, E55V, and D147Y. It should be appreciated, however, that any combination of corresponding mutations provided herein may be made in an adenosine deaminase (e.g., ecTadA). In some embodiments, an adenosine deaminase comprises one or more of the mutations provided herein, which identifies individual mutations and combinations of mutations made in ecTadA. In some embodiments, an adenosine deaminase comprises any mutation or combination of mutations provided herein.

In some embodiments, the adenosine deaminase comprises an L84X mutation in ecTadA SEQ ID NO: 278, or a corresponding mutation in another adenosine deaminase (e.g., SEQ ID NO: 279-291), where X indicates any amino acid other than the corresponding amino acid in the wild-type adenosine deaminase. In some embodiments, the adenosine deaminase comprises an L84F mutation in SEQ ID NO: 278, or a corresponding mutation in another adenosine deaminase (e.g., SEQ ID NO: 279-291).

In some embodiments, the adenosine deaminase comprises an H123X mutation in ecTadA SEQ ID NO: 278, or a corresponding mutation in another adenosine deaminase (e.g., SEQ ID NO: 279-291), where X indicates any amino acid other than the corresponding amino acid in the wild-type adenosine deaminase. In some embodiments, the adenosine deaminase comprises an H123Y mutation in SEQ ID NO: 278, or a corresponding mutation in another adenosine deaminase (e.g., SEQ ID NO: 279-291).

In some embodiments, the adenosine deaminase comprises an I156X mutation in ecTadA SEQ ID NO: 278, or a corresponding mutation in another adenosine deaminase (e.g., SEQ ID NO: 279-291), where X indicates any amino acid other than the corresponding amino acid in the wild-type adenosine deaminase. In some embodiments, the adenosine deaminase comprises an I156F mutation in SEQ ID NO: 278, or a corresponding mutation in another adenosine deaminase (e.g., SEQ ID NO: 279-291).

In some embodiments, the adenosine deaminase comprises one, two, three, four, five, six, or seven mutations selected from the group consisting of L84X, A106X, D108X, H123X, D147X, E155X, and I156X in SEQ ID NO: 278, or a corresponding mutation or mutations in another adenosine deaminase (e.g., SEQ ID NO: 279-291), where X indicates the presence of any amino acid other than the corresponding amino acid in the wild-type adenosine deaminase.

In some embodiments, the adenosine deaminase comprises one, two, three, four, five, six, or seven mutations selected from the group consisting of L84F, A106V, D108N, H123Y, D147Y, E155V, and I156F in SEQ ID NO: 278, or a corresponding mutation or mutations in another adenosine deaminase (e.g., SEQ ID NO: 279-291). In some embodiments, the adenosine deaminase comprises one, two, three, four, five, or six mutations selected from the group consisting of S2A, I49F, A106V, D108N, D147Y, and E155V in SEQ ID NO: 278, or a corresponding mutation or mutations in another adenosine deaminase (e.g., SEQ ID NO: 279-291). In some embodiments, the adenosine deaminase comprises one, two, three, four, or five, mutations selected from the group consisting of H8Y, A106T, D108N, N127S, and K160S in SEQ ID NO: 278, or a corresponding mutation or mutations in another adenosine deaminase (e.g., SEQ ID NO: 279-291).

In some embodiments, the adenosine deaminase comprises one or more of a W23X, H36X, N37X, P48X, I49X, R51X, N72X, L84X, S97X, A106X, D108X, H123X, G125X, A142X, S146X, D147X, R152X, E155X, I156X, K157X, and/or K161X mutation in SEQ ID NO: 278, or one or more corresponding mutations in another adenosine deaminase (e.g., SEQ ID NO: 279-291), where the presence of X indicates any amino acid other than the corresponding amino acid in the wild-type adenosine deaminase. In some embodiments, the adenosine deaminase comprises one or more of W23L, W23R, H36L, P48S, P48A, R51L, L84F, A106V, D108N, H123Y, A142N, S146C, D147Y, R152P, E155V, I156F, and/or K157N mutation in SEQ ID NO: 278, or one or more corresponding mutations in another adenosine deaminase (e.g., SEQ ID NO: 279-291). In some embodiments, the adenosine deaminase comprises one or more of the mutations provided herein corresponding to SEQ ID NO: 278, or one or more corresponding mutations in another adenosine deaminase (e.g., SEQ ID NO: 279-291).

In some embodiments, the adenosine deaminase comprises or consists of one or two mutations selected from A106X and D108X in SEQ ID NO: 278, or a corresponding mutation or mutations in another adenosine deaminase (e.g., SEQ ID NO: 279-291), where X indicates the presence of any amino acid other than the corresponding amino acid in the wild-type adenosine deaminase. In some embodiments, the adenosine deaminase comprises or consists of one or two mutations selected from A106V and D108N in SEQ ID NO: 278, or a corresponding mutation or mutations in another adenosine deaminase (e.g., SEQ ID NO: 279-291).

In some embodiments, the adenosine deaminase comprises or consists of one, two, three, or four mutations selected from A106X, D108X, D147X, and E155X in SEQ ID NO: 278, or a corresponding mutation or mutations in another adenosine deaminase (e.g., SEQ ID NO: 279-291), where X indicates the presence of any amino acid other than the corresponding amino acid in the wild-type adenosine deaminase. In some embodiments, the adenosine deaminase comprises or consists of one, two, three, or four mutations selected from A106V, D108N, D147Y, and E155V in SEQ ID NO: 278, or a corresponding mutation or mutations in another adenosine deaminase (e.g., SEQ ID NO: 279-291). In some embodiments, the adenosine deaminase comprises or consists of an A106V, D108N, D147Y, and E155V mutation in SEQ ID NO: 278, or corresponding mutations in another adenosine deaminase (e.g., SEQ ID NO: 279-291).

In some embodiments, the adenosine deaminase comprises one or more of the mutations provided herein corresponding to SEQ ID NO: 278, or one or more of the corresponding mutations in another deaminase. In some embodiments, the adenosine deaminase comprises or consists of a variant of SEQ ID NO: 278 provided herein, or the corresponding variant in another adenosine deaminase.

It should be appreciated that the adenosine deaminase (e.g., a first or second adenosine deaminase) may comprise one or more of the mutations provided in any of the adenosine deaminases (e.g., ecTadA adenosine deaminases) provided herein. In some embodiments, the adenosine deaminase comprises the combination of mutations of any of the adenosine deaminases (e.g., ecTadA adenosine deaminases) provided herein. For example, the adenosine deaminase may comprise the mutations W23R, H36L, P48A, R51L, L84F, A106V, D108N, H123Y, S146C, D147Y, R152P, E155V, I156F, and K157N (relative to SEQ ID NO: 278), which is shown as ABE7.10 provided herein. In some embodiments, the adenosine deaminase may comprise the mutations H36L, R51L, L84F, A106V, D108N, H123Y, S146C, D147Y, E155V, I156F, and K157N (relative to SEQ ID NO: 278).

In some embodiments, the adenosine deaminase comprises an amino acid sequence that is at least 60%, 65%, 70%, 75%, 80%, 85%, 90%, 95, 98%, 99%, or 99.5% identical to any one of SEQ ID NOs: 278-291, or any of the adenosine deaminases described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 278-291, or any of the adenosine deaminases described herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 166, identical contiguous amino acid residues as compared to any one of the amino acid sequences set forth in SEQ ID NOs: 278-291, or any of the adenosine deaminases described herein. In some embodiments, the adenosine deaminase comprises the amino acid sequence of any one of SEQ ID NOs: 278-291, or any of the adenosine deaminases described herein. In some embodiments, the adenosine deaminase consists of the amino acid sequence of any one of SEQ ID NOs: 278-291, or any of the adenosine deaminases described herein. The ecTadA sequences provided below are from ecTadA (SEQ ID NO: 278), absent the N-terminal methionine (M). For clarity, the amino acid numbering scheme used to identify the various amino acid mutations is derived from ecTadA (SEQ ID NO: 278) for E. coli TadA.

### Cytidine Deaminase Domains

In some embodiments, the disclosure provides base editors that comprise one or more cytidine deaminase domains. In some aspects, any of the disclosed base editors are capable of deaminating cytidine in a nucleic acid sequence (e.g., genomic DNA). As one example, any of the base editors described herein may be base editors, (e.g., cytidine base editors).

In some embodiments, the cytidine deaminase is an apolipoprotein B mRNA-editing complex (APOBEC) family deaminase. In some embodiments, the cytidine deaminase is an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, or an APOBEC4 deaminase. In some embodiments, the cytidine deaminase is an activation-induced deaminase (AID). In some embodiments, the deaminase is a Lamprey CDA1 (pmCDA1) deaminase. In some embodiments, the cytidine deaminase is from a human, chimpanzee, gorilla, monkey, cow, dog, rat, or mouse. In some embodiments, the deaminase is from a human. In some embodiments the deaminase is from a rat. In some embodiments, the cytidine deaminase is a human APOBEC1 deaminase. In some embodiments, the cytidine deaminase is pmCDA1. In some embodiments, the deaminase is human APOBEC3G. In some embodiments, the deaminase is a human APOBEC3G variant. In some embodiments, the deaminase is at least 80%, at least 85%, at least 90%, at least 92%, at least 95%, at least 96%, at least 97%, at least 98%, at least 99%, or at least 99.5% identical to any one of the APOBEC amino acid sequences set forth herein.

Additional exemplary cytidine deaminases from the APOBEC family include Pongo pygmaeus APOBEC1 (PpAPOBEC1), Rhinopithecus roxellana APOBEC3F (RrA3F), Alligator mississippiensis (AmAPOBEC1), and Sus scrofa APOBEC3B (SsAPOBEC3B), and variants thereof. For example, any one of the cytidine deaminases PpAPOBEC1, PpAPOBEC1 H122A, PpAPOBEC1 R33A, RrA3F, RrA3F F130L, AmAPOBEC1, SsAPOBEC3B, and SsAPOBEC3B R54Q, may be used with the editing methods provided herein. See Gaudelli et al. Nat. Commun. (2020) 11:2052 and International Publication No. WO 2021/041885, published Mar. 4, 2021, each of which is incorporated by reference herein.

Some exemplary suitable cytidine deaminases domains that can be fused to Cas9 domains according to aspects of this disclosure are provided below. It should be understood that the Specification also embraces other cytidine deaminases comprising an amino acid sequence having at least 80%, at least 85%, at least 90%, at least 95%, at least 98%, at least 99%, or at least 99.5% sequence identity to one of the following exemplary cytidine deaminases:

Human AID:
(SEQ ID NO: 292)
MDSLLMNRRKFLYQFKNVRWAKGRRETYLCYVVKR
RDSATSFSLDFGYLRNKNGCHVELLFLRYISDWDL
DPGRCYRVTWFTSWSPCYDCARHVADFLRGNPNLS
LRIFTARLYFCEDRKAEPEGLRRLHRAGVQIAIMT
FKDYFYCWNTFVENHERTFKAWEGLHENSVRLSRQ
LRRILLPLYEVDDLRDAFRTLGL
Mouse AID:
(SEQ ID NO: 293)
MDSLLMKQKKFLYHFKNVRWAKGRHETYLCYVVKR
RDSATSCSLDFGHLRNKSGCHVELLFLRYISDWDL
DPGRCYRVTWFTSWSPCYDCARHVAEFLRWNPNLS
LRIFTARLYFCEDRKAEPEGLRRLHRAGVQIGIMT
FKDYFYCWNTFVENRERTFKAWEGLHENSVRLTRQ
LRRILLPLYEVDDLRDAFRMLGF
Dog AID:
(SEQ ID NO: 294)
MDSLLMKQRKFLYHFKNVRWAKGRHETYLCYVVKR
RDSATSFSLDFGHLRNKSGCHVELLFLRYISDWDL
DPGRCYRVTWFTSWSPCYDCARHVADFLRGYPNLS
LRIFAARLYFCEDRKAEPEGLRRLHRAGVQIAIMT
FKDYFYCWNTFVENREKTFKAWEGLHENSVRLSRQ
LRRILLPLYEVDDLRDAFRTLGL
Bovine AID:
(SEQ ID NO: 295)
MDSLLKKQRQFLYQFKNVRWAKGRHETYLCYVVKR
RDSPTSFSLDFGHLRNKAGCHVELLFLRYISDWDL
DPGRCYRVTWFTSWSPCYDCARHVADFLRGYPNLS
LRIFTARLYFCDKERKAEPEGLRRLHRAGVQIAIM
TFKDYFYCWNTFVENHERTFKAWEGLHENSVRLSR
QLRRILLPLYEVDDLRDAFRTLGL
Rat:AID:
(SEQ ID NO: 296)
MAVGSKPKAALVGPHWERERIWCFLCSTGLGTQQT
GQTSRWLRPAATQDPVSPPRSLLMKQRKFLYHFKN
VRWAKGRHETYLCYVVKRRDSATSFSLDFGYLRNK
SGCHVELLFLRYISDWDLDPGRCYRVTWFTSWSPC
YDCARHVADFLRGNPNLSLRIFTARLTGWGALPAG
LMSPARPSDYFYCWNTFVENHERTFKAWEGLHENS
VRLSRRLRRILLPLYEVDDLRDAFRTLGL
Mouse APOBEC-3:
(SEQ ID NO: 297)
MGPFCLGCSHRKCYSPIRNLISQETFKFHFKNLGY
AKGRKDTFLCYEVTRKDCDSPVSLHHGVFKNKDNI
HAEICFLYWFHDKVLKVLSPREEFKITWYMSWSPC
FECAEQIVRFLATHHNLSLDIFSSRLYNVQDPETQ
QNLCRLVQEGAQVAAMDLYEFKKCWKKFVDNGGRR
FRPWKRLLTNFRYQDSKLQEILRPCYIPVPSSSSS
TLSNICLTKGLPETRFCVEGRRMDPLSEEEFYSQF
YNQRVKHLCYYHRMKPYLCYQLEQFNGQAPLKGCL
LSEKGKQHAEILFLDKIRSMELSQVTITCYLTWSP
CPNCAWQLAAFKRDRPDLILHIYTSRLYFHWKRPF
QKGLCSLWQSGILVDVMDLPQFTDCWTNFVNPKRP
FWPWKGLEIISRRTQRRLRRIKESWGLQDLVNDFG
NLQLGPPMS
Rat APOBEC-3:
(SEQ ID NO: 298)
MGPFCLGCSHRKCYSPIRNLISQETFKFHFKNLRY
AIDRKDTFLCYEVTRKDCDSPVSLHHGVFKNKDNI
HAEICFLYWFHDKVLKVLSPREEFKITWYMSWSPC
FECAEQVLRFLATHHNLSLDIFSSRLYNIRDPENQ
QNLCRLVQEGAQVAAMDLYEFKKCWKKFVDNGGRR
FRPWKKLLTNFRYQDSKLQEILRPCYIPVPSSSSS
TLSNICLTKGLPETRFCVERRRVHLLSEEEFYSQF
YNQRVKHLCYYHGVKPYLCYQLEQFNGQAPLKGCL
LSEKGKQHAEILFLDKIRSMELSQVIITCYLTWSP
CPNCAWQLAAFKRDRPDLILHIYTSRLYFHWKRPF
QKGLCSLWQSGILVDVMDLPQFTDCWTNFVNPKRP
FWPWKGLEIISRRTQRRLHRIKESWGLQDLVNDFG
NLQLGPPMS
Rhesus macaque APOBEC-3G:
(SEQ ID NO: 299)
MVEPMDPRTFVSNFNNRPILSGLNTVWLCCEVKTK
DPSGPPLDAKIFQGKVYSKAKYHPEMRFLRWFHKW
RQLHHDQEYKVTWYVSWSPCTRCANSVATFLAKDP
KVTLTIFVARLYYFWKPDYQQALRILCQKRGGPHA
TMKIMNYNEFQDCWNKFVDGRGKPFKPRNNLPKHY
TLLQATLGELLRHLMDPGTFTSNFNNKPWVSGQHE
TYLCYKVERLHNDTWVPLNQHRGFLRNQAPNIHGF
PKGRHAELCFLDLIPFWKLDGQQYRVTCFTSWSPC
FSCAQEMAKFISNNEHVSLCIFAARIYDDQGRYQE
GLRALHRDGAKIAMMNYSEFEYCWDTFVDRQGRPF
QPWDGLDEHSQALSGRLRAI
Chimpanzee APOBEC-3G:
(SEQ ID NO: 300)
MKPHFRNPVERMYQDTFSDNFYNRPILSHRNTVWL
CYEVKTKGPSRPPLDAKIFRGQVYSKLKYHPEMRF
FHWFSKWRKLHRDQEYEVTWYISWSPCTKCTRDVA
TFLAEDPKVTLTIFVARLYYFWDPDYQEALRSLCQ
KRDGPRATMKIMNYDEFQHCWSKFVYSQRELFEPW
NNLPKYYILLHIMLGEILRHSMDPPTFTSNFNNEL
WVRGRHETYLCYEVERLHNDTWVLLNQRRGFLCNQ
APHKHGFLEGRHAELCFLDVIPFWKLDLHQDYRVT
CFTSWSPCFSCAQEMAKFISNNKHVSLCIFAARIY
DDQGRCQEGLRTLAKAGAKISIMTYSEFKHCWDTF
VDHQGCPFQPWDGLEEHSQALSGRLRAILQNQGN
Green monkey APOBEC-3G:
(SEQ ID NO: 301)
MNPQIRNMVEQMEPDIFVYYFNNRPILSGRNTVWL
CYEVKTKDPSGPPLDANIFQGKLYPEAKDHPEMKF
LHWFRKWRQLHRDQEYEVTWYVSWSPCTRCANSVA
TFLAEDPKVTLTIFVARLYYFWKPDYQQALRILCQ
ERGGPHATMKIMNYNEFQHCWNEFVDGQGKPFKPR
KNLPKHYTLLHATLGELLRHVMDPGTFTSNFNNKP
WVSGQRETYLCYKVERSHNDTWVLLNQHRGFLRNQ
APDRHGFPKGRHAELCFLDLIPFWKLDDQQYRVTC
FTSWSPCFSCAQKMAKFISNNKHVSLCIFAARIYD
DQGRCQEGLRTLHRDGAKIAVMNYSEFEYCWDTFV
DRQGRPFQPWDGLDEHSQALSGRLRAI
Human APOBEC-3G:
(SEQ ID NO: 302)
MKPHFRNTVERMYRDTFSYNFYNRPILSRRNTVWL
CYEVKTKGPSRPPLDAKIFRGQVYSELKYHPEMRF
FHWFSKWRKLHRDQEYEVTWYISWSPCTKCTRDMA
TFLAEDPKVTLTIFVARLYYFWDPDYQEALRSLCQ
KRDGPRATMKIMNYDEFQHCWSKFVYSQRELFEPW
NNLPKYYILLHIMLGEILRHSMDPPTFTFNFNNEP
WVRGRHETYLCYEVERMHNDTWVLLNQRRGFLCNQ
APHKHGFLEGRHAELCFLDVIPFWKLDLDQDYRVT
CFTSWSPCFSCAQEMAKFISKNKHVSLCIFTARIY
DDQGRCQEGLRTLAEAGAKISIMTYSEFKHCWDTF
VDHQGCPFQPWDGLDEHSQDLSGRLRAILQNQEN
Human APOBEC-3F:
(SEQ ID NO: 303)
MKPHFRNTVERMYRDTFSYNFYNRPILSRRNTVWL
CYEVKTKGPSRPRLDAKIFRGQVYSQPEHHAEMCF
LSWFCGNQLPAYKCFQITWFVSWTPCPDCVAKLAE
FLAEHPNVTLTISAARLYYYWERDYRRALCRLSQA
GARVKIMDDEEFAYCWENFVYSEGQPFMPWYKFDD
NYAFLHRTLKEILRNPMEAMYPHIFYFHFKNLRKA
YGRNESWLCFTMEVVKHHSPVSWKRGVFRNQVDPE
THCHAERCFLSWFCDDILSPNTNYEVTWYTSWSPC
PECAGEVAEFLARHSNVNLTIFTARLYYFWDTDYQ
EGLRSLSQEGASVEIMGYKDFKYCWENFVYNDDEP
FKPWKGLKYNFLFLDSKLQEILE
Human APOBEC-3B:
(SEQ ID NO: 304)
MNPQIRNPMERMYRDTFYDNFENEPILYGRSYTWL
CYEVKIKRGRSNLLWDTGVFRGQVYFKPQYHAEMC
FLSWFCGNQLPAYKCFQITWFVSWTPCPDCVAKLA
EFLSEHPNVTLTISAARLYYYWERDYRRALCRLSQ
AGARVTIMDYEEFAYCWENFVYNEGQQFMPWYKFD
ENYAFLHRTLKEILRYLMDPDTFTFNFNNDPLVLR
RRQTYLCYEVERLDNGTWVLMDQHMGFLCNEAKNL
LCGFYGRHAELRFLDLVPSLQLDPAQIYRVTWFIS
WSPCFSWGCAGEVRAFLQENTHVRLRIFAARIYDY
DPLYKEALQMLRDAGAQVSIMTYDEFEYCWDTFVY
RQGCPFQPWDGLEEHSQALSGRLRAILQNQGN
Rat APOBEC-3B:
(SEQ ID NO: 305)
MQPQGLGPNAGMGPVCLGCSHRRPYSPIRNPLKKL
YQQTFYFHFKNVRYAWGRKNNFLCYEVNGMDCALP
VPLRQGVFRKQGHIHAELCFIYWFHDKVLRVLSPM
EEFKVTWYMSWSPCSKCAEQVARFLAAHRNLSLAI
FSSRLYYYLRNPNYQQKLCRLIQEGVHVAAMDLPE
FKKCWNKFVDNDGQPFRPWMRLRINFSFYDCKLQE
IFSRMNLLREDVFYLQFNNSHRVKPVQNRYYRRKS
YLCYQLERANGQEPLKGYLLYKKGEQHVEILFLEK
MRSMELSQVRITCYLTWSPCPNCARQLAAFKKDHP
DLILRIYTSRLYFYWRKKFQKGLCTLWRSGIHVDV
MDLPQFADCWTNFVNPQRPFRPWNELEKNSWRIQR
RLRRIKESWGL
Bovine APOBEC-3B:
(SEQ ID NO: 306)
DGWEVAFRSGTVLKAGVLGVSMTEGWAGSGHPGQG
ACVWTPGTRNTMNLLREVLFKQQFGNQPRVPAPYY
RRKTYLCYQLKQRNDLTLDRGCFRNKKQRHAEIRF
IDKINSLDLNPSQSYKIICYITWSPCPNCANELVN
FITRNNHLKLEIFASRLYFHWIKSFKMGLQDLQNA
GISVAVMTHTEFEDCWEQFVDNQSRPFQPWDKLEQ
YSASIRRRLQRILTAPI
Chimpanzee APOBEC-3B:
(SEQ ID NO: 307)
MNPQIRNPMEWMYQRTFYYNFENEPILYGRSYTWL
CYEVKIRRGHSNLLWDTGVFRGQMYSQPEHHAEMC
FLSWFCGNQLSAYKCFQITWFVSWTPCPDCVAKLA
KFLAEHPNVTLTISAARLYYYWERDYRRALCRLSQ
AGARVKIMDDEEFAYCWENFVYNEGQPFMPWYKFD
DNYAFLHRTLKEIIRHLMDPDTFTFNFNNDPLVLR
RHQTYLCYEVERLDNGTWVLMDQHMGFLCNEAKNL
LCGFYGRHAELRFLDLVPSLQLDPAQIYRVTWFIS
WSPCFSWGCAGQVRAFLQENTHVRLRIFAARIYDY
DPLYKEALQMLRDAGAQVSIMTYDEFEYCWDTFVY
RQGCPFQPWDGLEEHSQALSGRLRAILQVRASSLC
MVPHRPPPPPQSPGPCLPLCSEPPLGSLLPTGRPA
PSLPFLLTASFSFPPPASLPPLPSLSLSPGHLPVP
SFHSLTSCSIQPPCSSRIRETEGWASVSKEGRDLG
Human APOBEC-3C:
(SEQ ID NO: 308)
MNPQIRNPMKAMYPGTFYFQFKNLWEANDRNETWL
CFTVEGIKRRSVVSWKTGVFRNQVDSETHCHAERC
FLSWFCDDILSPNTKYQVTWYTSWSPCPDCAGEVA
EFLARHSNVNLTIFTARLYYFQYPCYQEGLRSLSQ
EGVAVEIMDYEDFKYCWENFVYNDNEPFKPWKGLK
TNFRLLKRRLRESLQ
Gorilla APOBEC3C:
(SEQ ID NO: 309)
MNPQIRNPMKAMYPGTFYFQFKNLWEANDRNETWL
CFTVEGIKRRSVVSWKTGVFRNQVDSETHCHAERC
FLSWFCDDILSPNTNYQVTWYTSWSPCPECAGEVA
EFLARHSNVNLTIFTARLYYFQDTDYQEGLRSLSQ
EGVAVKIMDYKDFKYCWENFVYNDDEPFKPWKGLK
YNFRFLKRRLQEILE
Human APOBEC-3A:
(SEQ ID NO: 310)
MEASPASGPRHLMDPHIFTSNFNNGIGRHKTYLCY
EVERLDNGTSVKMDQHRGFLHNQAKNLLCGFYGRH
AELRFLDLVPSLQLDPAQIYRVTWFISWSPCFSWG
CAGEVRAFLQENTHVRLRIFAARIYDYDPLYKEAL
QMLRDAGAQVSIMTYDEFKHCWDTFVDHQGCPFQP
WDGLDEHSQALSGRLRAILQNQGN
Rhesus macaque APOBEC-3A:
(SEQ ID NO: 311)
MDGSPASRPRHLMDPNTFTFNFNNDLSVRGRHQTY
LCYEVERLDNGTWVPMDERRGFLCNKAKNVPCGDY
GCHVELRFLCEVPSWQLDPAQTYRVTWFISWSPCF
RRGCAGQVRVFLQENKHVRLRIFAARIYDYDPLYQ
EALRTLRDAGAQVSIMTYEEFKHCWDTFVDRQGRP
FQPWDGLDEHSQALSGRLRAILQNQGN
Bovine APOBEC-3A:
(SEQ ID NO: 312)
MDEYTFTENFNNQGWPSKTYLCYEMERLDGDATIP
LDEYKGFVRNKGLDQPEKPCHAELYFLGKIHSWNL
DRNQHYRLTCFISWSPCYDCAQKLTTFLKENHHIS
LHILASRIYTHNRFGCHQSGLCELQAAGARITIMT
FEDFKHCWETFVDHKGKPFQPWEGLNVKSQALCTE
LQAILKTQQN
Human APOBEC-3H:
(SEQ ID NO: 313)
MALLTAETFRLQFNNKRRLRRPYYPRKALLCYQLT
PQNGSTPTRGYFENKKKCHAEICFINEIKSMGLDE
TQCYQVTCYLTWSPCSSCAWELVDFIKAHDHLNLG
IFASRLYYHWCKPQQKGLRLLCGSQVPVEVMGFPK
FADCWENFVDHEKPLSFNPYKMLEELDKNSRAIKR
RLERIKIPGVRAQGRYMDILCDAEV
Rhesus macaque APOBEC-3H:
(SEQ ID NO: 314)
MALLTAKTFSLQFNNKRRVNKPYYPRKALLCYQLT
PQNGSTPTRGHLKNKKKDHAEIRFINKIKSMGLDE
TQCYQVTCYLTWSPCPSCAGELVDFIKAHRHLNLR
IFASRLYYHWRPNYQEGLLLLCGSQVPVEVMGLPE
FTDCWENFVDHKEPPSFNPSEKLEELDKNSQAIKR
RLERIKSRSVDVLENGLRSLQLGPVTPSSSIRNSR
Human APOBEC-3D:
(SEQ ID NO: 315)
MNPQIRNPMERMYRDTFYDNFENEPILYGRSYTWL
CYEVKIKRGRSNLLWDTGVFRGPVLPKRQSNHRQE
VYFRFENHAEMCFLSWFCGNRLPANRRFQITWFVS
WNPCLPCVVKVTKFLAEHPNVTLTISAARLYYYRD
RDWRWVLLRLHKAGARVKIMDYEDFAYCWENFVCN
EGQPFMPWYKFDDNYASLHRTLKEILRNPMEAMYP
HIFYFHFKNLLKACGRNESWLCFTMEVTKHHSAVF
RKRGVFRNQVDPETHCHAERCFLSWFCDDILSPNT
NYEVTWYTSWSPCPECAGEVAEFLARHSNVNLTIF
TARLCYFWDTDYQEGLCSLSQEGASVKIMGYKDFV
SCWKNFVYSDDEPFKPWKGLQTNFRLLKRRLREIL
Q
Human APOBEC-1:
(SEQ ID NO: 316)
MTSEKGPSTGDPTLRRRIEPWEFDVFYDPRELRKE
ACLLYEIKWGMSRKIWRSSGKNTTNHVEVNFIKKF
TSERDFHPSMSCSITWFLSWSPCWECSQAIREFLS
RHPGVTLVIYVARLFWHMDQQNRQGLRDLVNSGVT
IQIMRASEYYHCWRNFVNYPPGDEAHWPQYPPLWM
MLYALELHCIILSLPPCLKISRRWQNHLTFFRLHL
QNCHYQTIPPHILLATGLIHPSVAWR
Mouse APOBEC-1:
(SEQ ID NO: 317)
MSSETGPVAVDPTLRRRIEPHEFEVFFDPRELRKE
TCLLYEINWGGRHSVWRHTSQNTSNHVEVNFLEKF
TTERYFRPNTRCSITWFLSWSPCGECSRAITEFLS
RHPYVTLFIYIARLYHHTDQRNRQGLRDLISSGVT
IQIMTEQEYCYCWRNFVNYPPSNEAYWPRYPHLWV
KLYVLELYCIILGLPPCLKILRRKQPQLTFFTITL
QTCHYQRIPPHLLWATGLK
Rat APOBEC-1:
(SEQ ID NO: 318)
MSSETGPVAVDPTLRRRIEPHEFEVFFDPRELRKE
TCLLYEINWGGRHSIWRHTSQNTNKHVEVNFIEKF
TTERYFCPNTRCSITWFLSWSPCGECSRAITEFLS
RYPHVTLFIYIARLYHHADPRNRQGLRDLISSGVT
IQIMTEQESGYCWRNFVNYSPSNEAHWPRYPHLWV
RLYVLELYCIILGLPPCLNILRRKQPQLTFFTIAL
QSCHYQRLPPHILWATGLK
Human APOBEC-2:
(SEQ ID NO: 319)
MAQKEEAAVATEAASQNGEDLENLDDPEKLKELIE
LPPFEIVTGERLPANFFKFQFRNVEYSSGRNKTFL
CYVVEAQGKGGQVQASRGYLEDEHAAAHAEEAFFN
TILPAFDPALRYNVTWYVSSSPCAACADRIIKTLS
KTKNLRLLILVGRLFMWEEPEIQAALKKLKEAGCK
LRIMKPQDFEYVWQNFVEQEEGESKAFQPWEDIQE
NFLYYEEKLADILK
Mouse APOBEC-2:
(SEQ ID NO: 320)
MAQKEEAAEAAAPASQNGDDLENLEDPEKLKELID
LPPFEIVTGVRLPVNFFKFQFRNVEYSSGRNKTFL
CYVVEVQSKGGQAQATQGYLEDEHAGAHAEEAFFN
TILPAFDPALKYNVTWYVSSSPCAACADRILKTLS
KTKNLRLLILVSRLFMWEEPEVQAALKKLKEAGCK
LRIMKPQDFEYIWQNFVEQEEGESKAFEPWEDIQE
NFLYYEEKLADILK
Rat APOBEC-2:
(SEQ ID NO: 321)
MAQKEEAAEAAAPASQNGDDLENLEDPEKLKELID
LPPFEIVTGVRLPVNFFKFQFRNVEYSSGRNKTFL
CYVVEAQSKGGQVQATQGYLEDEHAGAHAEEAFFN
TILPAFDPALKYNVTWYVSSSPCAACADRILKTLS
KTKNLRLLILVSRLFMWEEPEVQAALKKLKEAGCK
LRIMKPQDFEYLWQNFVEQEEGESKAFEPWEDIQE
NFLYYEEKLADILK
Bovine APOBEC-2:
(SEQ ID NO: 322)
MAQKEEAAAAAEPASQNGEEVENLEDPEKLKELIE
LPPFEIVTGERLPAHYFKFQFRNVEYSSGRNKTFL
CYVVEAQSKGGQVQASRGYLEDEHATNHAEEAFFN
SIMPTFDPALRYMVTWYVSSSPCAACADRIVKTLN
KTKNLRLLILVGRLFMWEEPEIQAALRKLKEAGCR
LRIMKPQDFEYIWQNFVEQEEGESKAFEPWEDIQE
NFLYYEEKLADILK
Petromyzon marinus CD A1 (pmCDAl):
(SEQ ID NO: 323)
MTDAEYVRIHEKLDIYTFKKQFFNNKKSVSHRCYV
LFELKRRGERRACFWGYAVNKPQSGTERGIHAEIF
SIRKVEEYLRDNPGQFTINWYSSWSPCADCAEKIL
EWYNQELRGNGHTLKIWACKLYYEKNARNQIGLWN
LRDNGVGLNVMVSEHYQCCRKIFIQSSHNQLNENR
WLEKTLKRAEKRRSELSIMIQVKILHTTKSPAV
Human APOBEC3G D316R_D317R:
(SEQ ID NO: 324)
MKPHFRNTVERMYRDTFSYNFYNRPILSRRNTVWL
CYEVKTKGPSRPPLDAKIFRGQVYSELKYHPEMRF
FHWFSKWRKLHRDQEYEVTWYI
SWSPCTKCTRDMATFLAEDPKVTLTIFVARLYYFW
DPDYQEALRSLCQKRDGPRATMKIMNYDEFQHCWS
KFVYSQRELFEPWNNLPKYYILLHIMLGEILRHSM
DPPTFTFNFNNEPWVRGRHETYLCYEVERMHNDTW
VLLNQRRGFLCNQAPHKHGFLEGRHAELCFLDVIP
FWKLDLDQDYRVTCFTSWSPCFSCAQEMAKFISKN
KHVSLCIFTARIYRRQGRCQEGLRTLAEAGAKISI
MTYSEFKHCWDTFVDHQGCPFQPWDGLDEHSQDLS
GRLRAILQNQEN
Human APOBEC3G chain A:
(SEQ ID NO: 325)
MDPPTFTFNFNNEPWVRGRHETYLCYEVERMHNDT
WVLLNQRRGFLCNQAPHKHGFLEGRHAELCFLDVI
PFWKLDLDQDYRVTCFTSWSPCFSCAQEMAKFISK
NKHVSLCIFTARIYDDQGRCQEGLRTLAEAGAKIS
IMTYSEFKHCWDTFVDHQGCPFQPWDGLDEHSQDL
SGRLRAILQ
Human APOBEC3G chain A D120R_D121R:
(SEQ ID NO: 326)
MDPPTFTFNFNNEPWVRGRHETYLCYEVERMHNDT
WVLLNQRRGFLCNQAPHKHGFLEGRHAELCFLDVI
PFWKLDLDQDYRVTCFTSWSPCFSCAQEMAKFISK
NKHVSLCIFTARIYRRQGRCQEGLRTLAEAGAKIS
IMTYSEFKHCWDTFVDHQGCPFQPWDGLDEHSQDL
SGRLRAILQ

Any of the aforementioned DNA effector domains may be subjected to a continuous evolution process (e.g., PACE) or may be otherwise further evolved using a mutagenesis methodology known in the art.

In some embodiments, the cytidine deaminase is an apolipoprotein B mRNA-editing complex (APOBEC) family deaminase. In some embodiments, the deaminase is an APOBEC1 deaminase. In some embodiments, the deaminase is an APOBEC2 deaminase. In some embodiments, the deaminase is an APOBEC3 deaminase. In some embodiments, the deaminase is an APOBEC3A deaminase. In some embodiments, the deaminase is an APOBEC3B deaminase. In some embodiments, the deaminase is an APOBEC3C deaminase. In some embodiments, the deaminase is an APOBEC3D deaminase. In some embodiments, the deaminase is an APOBEC3E deaminase. In some embodiments, the deaminase is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). In some embodiments, the deaminase is a vertebrate deaminase. In some embodiments, the deaminase is an invertebrate deaminase. In some embodiments, the deaminase is a human, chimpanzee, gorilla, monkey, cow, dog, rat, or mouse deaminase. In some embodiments, the deaminase is a human deaminase. In some embodiments, the deaminase is a rat deaminase, e.g., rAPOBEC1.

Some aspects of the disclosure are based on the recognition that modulating the deaminase domain catalytic activity of any of the base editors described herein, for example by making point mutations in the deaminase domain, affect the processivity of the base editors (e.g., base editors). For example, mutations that reduce, but do not eliminate, the catalytic activity of a deaminase domain within a base editor can make it less likely that the deaminase domain will catalyze the deamination of a residue adjacent to a target residue, thereby narrowing the deamination window. The ability to narrow the deamination window may prevent unwanted deamination of residues adjacent of specific target residues, which may decrease or prevent off-target effects.

In some embodiments, any of the base editors described herein comprise a deaminase domain (e.g., a cytidine deaminase domain) that has reduced catalytic deaminase activity. In some embodiments, any of the base editors described herein comprise a deaminase domain (e.g., a cytidine deaminase domain) that has a reduced catalytic deaminase activity as compared to an appropriate control. For example, the appropriate control may be the deaminase activity of the deaminase prior to introducing one or more mutations into the deaminase. In other embodiments, the appropriate control may be a wild-type deaminase. In some embodiments, the appropriate control is a wild-type apolipoprotein B mRNA-editing complex (APOBEC) family deaminase. In some embodiments, the appropriate control is an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, or an APOBEC3H deaminase. In some embodiments, the appropriate control is an activation induced deaminase (AID). In some embodiments, the appropriate control is a cytidine deaminase 1 from Petromyzon marinus (pmCDA1). In some embodiments, the deaminse domain may be a deaminase domain that has at least 1%, at least 5%, at least 15%, at least 20%, at least 25%, at least 30%, at least 40%, at least 50%, at least 60%, at lest 70%, at least 80%, at least 90%, or at least 95% less catalytic deaminase activity as compared to an appropriate control.

The apolipoprotein B mRNA-editing complex (APOBEC) family of cytidine deaminase enzymes encompasses eleven proteins that serve to initiate mutagenesis in a controlled and beneficial manner. One family member, activation-induced cytidine deaminase (AID), is responsible for the maturation of antibodies by converting cytosines in ssDNA to uracils in a transcription-dependent, strand-biased fashion. The apolipoprotein B editing complex 3 (APOBEC3) enzyme provides protection to human cells against a certain HIV-1 strain via the deamination of cytosines in reverse-transcribed viral ssDNA. These proteins all require a Zn2+-coordinating motif (His-X-Glu-X23-26-Pro-Cys-X2-4-Cys; (SEQ ID NO: 402) and bound water molecule for catalytic activity. The Glu residue acts to activate the water molecule to a zinc hydroxide for nucleophilic attack in the deamination reaction. Each family member preferentially deaminates at its own particular “hotspot”, ranging from WRC (W is A or T, R is A or G) for hAID, to TTC for hAPOBEC3F. A recent crystal structure of the catalytic domain of APOBEC3G revealed a secondary structure comprised of a five-stranded (3-sheet core flanked by six a-helices, which is believed to be conserved across the entire family. The active center loops have been shown to be responsible for both ssDNA binding and in determining “hotspot” identity. Overexpression of these enzymes has been linked to genomic instability and cancer, thus highlighting the importance of sequence-specific targeting.

Some aspects of this disclosure relate to the recognition that the activity of cytidine deaminase enzymes such as APOBEC enzymes can be directed to a specific site in genomic DNA. Without wishing to be bound by any particular theory, advantages of using Cas9 as a recognition agent include (1) the sequence specificity of Cas9 can be easily altered by simply changing the sgRNA sequence; and (2) Cas9 binds to its target sequence by denaturing the dsDNA, resulting in a stretch of DNA that is single-stranded and therefore a viable substrate for the deaminase. It should be understood that other catalytic domains, or catalytic domains from other deaminases, can also be used to generate base editors with Cas9, and that the disclosure is not limited in this regard.

Some aspects of this disclosure are based on the recognition that Cas9:deaminase base editors can efficiently deaminate nucleotides. In view of the results provided herein regarding the nucleotides that can be targeted by Cas9:deaminase base editors, a person of skill in the art will be able to design suitable guide RNAs to target the base editors to a target sequence that comprises a nucleotide to be deaminated.

In certain embodiments, the reference cytidine deaminase domain comprises a “FERNY” polypeptide having an amino acid sequence according to SEQ ID NO: 327 or an amino acid sequence that is at least 60%, 65%, 70%, 75%, 80%, 85%, 90%, 95, 98%, 99%, or 99.5% identical to SEQ ID NO: 327, as follows:

(SEQ ID NO: 327)
MFERNYDPRELRKETYLLYEIKWGKSGKLWRHWCQ
NNRTQHAEVYFLENIFNARRFNPSTHCSITWYLSW
SPCAECSQKIVDFLKEHPNVNLEIYVARLYYHEDE
RNRQGLRDLVNSGVTIRIMDLPDYNYCWKTFVSDQ
GGDEDYWPGHFAPWIKQYSLKL

In certain other embodiment, the evolved cytidine deaminase domain (i.e., as a result of the continuous evolution process described herein) comprises a “evoFERNY” polypeptide having an amino acid sequence according to SEQ ID NO: 328 or an amino acid sequence that is at least 60%, 65%, 70%, 75%, 80%, 85%, 90%, 95, 98%, 99%, or 99.5% identical to SEQ ID NO: 328, comprising an H102P and D104N substitutions, as follows:

(SEQ ID NO: 328)
MFERNYDPRELRKETYLLYEIKWGKSGKLWRHWCQ
NNRTQHAEVYFLENIFNARRFNPSTHCSITWYLSW
SPCAECSQKIVDFLKEHPNVNLEIYVARLYYPENE
RNRQGLRDLVNSGVTIRIMDLPDYNYCWKTFVSDQ
GGDEDYWPGHFAPWIKQYSLKL

In other embodiments, the reference cytidine deaminase domain comprises a “Rat APOBEC-1” polypeptide having an amino acid sequence according to SEQ ID NO: 318, or an amino acid sequence that is at least 60%, 65%, 70%, 75%, 80%, 85%, 90%, 95, 98%, 99%, or 99.5% identical to SEQ ID NO: 318.

In certain other embodiment, the evolved cytidine deaminase domain (i.e., as a result of the continuous evolution process described herein) comprises a “evoAPOBEC” polypeptide having an amino acid sequence according to SEQ ID NO: 330, or an amino acid sequence that is at least 60%, 65%, 70%, 75%, 80%, 85%, 90%, 95, 98%, 99%, or 99.5% identical to SEQ ID NO: 330, and comprising substitutions E4K; H109N; H122L; D124N; R154H; A1655; P2015; F2055, as follows:

(SEQ ID NO: 330)
MSSKTGPVAVDPTLRRRIEPHEFEVFFDPRELRKE
TCLLYEINWGGRHSIWRHTSQNTNKHVEVNFIEKF
TTERYFCPNTRCSITWFLSWSPCGECSRAITEFLS
RYPNVTLFIYIARLYHLANPRNRQGLRDLISSGVT
IQIMTEQESGYCWHNFVNYSPSNESHWPRYPHLWV
RLYVLELYCIILGLPPCLNILRRKQSQLTSFTIAL
QSCHYQRLPPHILWATGLK

In still other embodiments, the reference cytidine deaminase domain comprises a “Petromyzon marinus CDA1 (pmCDA1)” polypeptide having an amino acid sequence according to SEQ ID NO: 323, or an amino acid sequence that is at least 60%, 65%, 70%, 75%, 80%, 85%, 90%, 95, 98%, 99%, or 99.5% identical to SEQ ID NO: 323.

In other embodiment, the evolved cytidine deaminase domain (i.e., as a result of the continuous evolution process described herein) comprises a “evoCDA” polypeptide having an amino acid sequence according to SEQ ID NO: 132 or an amino acid sequence that is at least 60%, 65%, 70%, 75%, 80%, 85%, 90%, 95, 98%, 99%, or 99.5% identical to SEQ ID NO: 332 and comprising substitutions F23S; A123V; I195F, as follows:

(SEQ ID NO: 332)
MTDAEYVRIHEKLDIYTFKKQFSNNKKSVSHRCYV
LFELKRRGERRACFWGYAVNKPQSGTERGIHAEIF
SIRKVEEYLRDNPGQFTINWYSSWSPCADCAEKIL
EWYNQELRGNGHTLKIWVCKLYYEKNARNQIGLWN
LRDNGVGLNVMVSEHYQCCRKIFIQSSHNQLNENR
WLEKTLKRAEKRRSELSIMFQVKILHTTSPAV

In yet other embodiments, the reference cytidine deaminase domain comprises a “Anc689 APOBEC” polypeptide having an amino acid sequence according to SEQ ID NO: 133 or an amino acid sequence that is at least 60%, 65%, 70%, 75%, 80%, 85%, 90%, 95, 98%, 99%, or 99.5% identical to SEQ ID NO: 333, as follows:

(SEQ ID NO: 333)
MSSETGPVAVDPTLRRRIEPHEFEVFFDPRELRKE
TCLLYEIKWGTSHKIWRHSSKNTTKHVEVNFIEKF
TSERHFCPSTSCSITWFLSWSPCGECSKAITEFLS
QHPNVTLVIYVARLYHHMDQQNRQGLRDLVNSGVT
IQIMTAPEYDYCWRNFVNYPPGKEAHWPRYPPLWM
KLYALELHAGILGLPPCLNILRRKQPQLTFFTIAL
QSCHYQRLPPHILWATGLK

In other embodiments, the evolved cytidine deaminase domain (i.e., as a result of the continuous evolution process described herein) comprises a “evoAnc689 APOBEC” polypeptide having an amino acid sequence according to SEQ ID NO: 334 or an amino acid sequence that is at least 60%, 65%, 70%, 75%, 80%, 85%, 90%, 95, 98%, 99%, or 99.5% identical to SEQ ID NO: 334 and comprising substitutions E4K; H122L; D124N; R154H; A1655; P2015; F2055, as follows:

(SEQ ID NO: 334)
MSSKTGPVAVDPTLRRRIEPHEFEVFFDPRELRKE
TCLLYEIKWGTSHKTWRHSSKNTTKHVEVNFIEKF
TSERHFCPSTSCSITWFLSWSPCGECSKAITEFLS
QHPNVTLVIYVARLYHLMNQQNRQGLRDLVNSGVT
IQIMTAPEYDYCWHNFVNYPPGKESHWPRYPPLWM
KLYALELHAGILGLPPCLNILRRKQSQLTSFTIAL
QSCHYQRLPPHILWATGLK.

In some embodiments, the cytidine deaminase domain comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 292-326, and SEQ ID NOs: 327, 328, 330, and 332-334, or to any of the cytidine deaminases described herein. The disclosure provides any deaminase domains with a certain percent identity plus any of the mutations or combinations thereof described herein. In exemplary embodiments, the cytosine deaminase domain comprises an amino acid sequence having at least 90%, at least 95%, at least 98%, or at least 99% identity to any one of SEQ ID NOs: 292-326. In some embodiments, the cytosine deaminase domain comprises the sequence of any one of SEQ ID NOs: 292-326, e.g., SEQ ID NO: 318 (rat APOBEC-1, or rAPOBEC1).

In some embodiments, the cytidine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acids that differ relative to any one of the amino acid sequences set forth in SEQ ID NOs: 292-326 and SEQ ID NOs: 327, 328, 330, and 332-334, or any of the cytidine deaminases described herein. These differences may comprise amino acids that have been inserted, deleted, or substituted relative to the amino acid sequence of any of SEQ ID NOs: 292-326 and SEQ ID NOs: 327, 328, 330, and 332-334. In some embodiments, any of the adenosine deaminase domains described herein may comprise an amino acid sequence having 1, 2, 3, 4, 5, 6, 7, 8, 9, 10, 11, 12, 13, 14, 15, 16, 17, 18, 19, 20, 25, 30, or more than 30 amino acids that differ relative to the amino acid sequence of a wild type cytidine deaminase (e.g., rAPOBEC1). In some embodiments, the adenosine deaminase domains comprise truncations at the N-terminus or C-terminus relative to a wild-type cytidine deaminase.

In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences set forth in SEQ ID NOs: 292-326 and SEQ ID NOs: 327, 328, 330, and 332-334, or any of the cytidine deaminases described herein.

In some aspects, the specification provides evolved cytidine deaminases which are used to construct base editors that have improved properties. For example, evolved cytidine deaminases, such as those provided herein, are capable of improving base editing efficiency and/or improving the ability of base editors to more efficiently edit bases regardless of the surrounding sequence. For example, in some aspects the disclosure provides evolved APOBEC deaminases (e.g., evolved rAPOBEC1) with improved base editing efficiency in the context of a 5′-G-3′ when it is 5′ to a target base (e.g., C). In some embodiments, the disclosure provides base editors comprising any of the evolved cytidine deaminases described herein. It should be appreciated that any of the evolved cydidine deaminases described herein may be used as a deaminase in a base editor protein, such as any of the base editors described herein. It should also be appreciated that the disclosure contemplates cytidine deaminases having any of the mutations provided herein, for example any of the mutations described in the Examples section.

### Other Functional Domains

In various embodiments, the base editors and their various components may comprise additional functional moieities, such as, but not limited to, linkers, uracil glycosylase inhibitors, nuclear localization signals, split-intein sequences (to join split proteins, such as split napDNAbps, split adenosine deaminases, split cytidine deaminases, split CBEs, or split ABEs), and RNA-protein recruitment domains (such as, MS2 tagging system).

(1) Linkers

In certain embodiments, linkers may be used to link any of the protein or protein domains described herein (e.g., a deaminase domain and a Cas9 domain). The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is an amino acid or a plurality of amino acids (e.g., a peptide or protein). In some embodiments, the linker is a bond e.g., a covalent bond), an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35-40, 40-45, 45-50, 50-60, 60-70, 70-80, 80-90, 90-100, 100-110, 110-120, 120-130, 130-140, 140-150, or 150-200 amino acids in length. Longer or shorter linkers are also contemplated. In some embodiments, a linker comprises the amino acid sequence SGSETPGTSESATPES (SEQ ID NO: 135), which may also be referred to as the XTEN linker. In some embodiments, the linker is 32 amino acids in length. In some embodiments, the linker comprises the amino acid sequence (SGGS)₂-SGSETPGTSESATPES-(SGGS)₂ (SEQ ID NO: 136), which may also be referred to as (SGGS)₂-XTEN-(SGGS)₂ (SEQ ID NO: 136). In some embodiments, the linker comprises the amino acid sequence, wherein n is 0, 1, 2, 3, 4, 5, 6, 7, 8, 9, or 10. In some embodiments, a linker comprises the amino acid sequence SGGS (SEQ ID NO: 137). In some embodiments, a linker comprises (SGGS)ₙ (SEQ ID NO: 137), (GGGS)ₙ (SEQ ID NO: 138), (GGGGS). (SEQ ID NO: 139), (G). (SEQ ID NO: 140), (EAAAK)ₙ (SEQ ID NO: 141), (SGGS).-SGSETPGTSESATPES-(SGGS)ₙ-(SEQ ID NO: 142), (GGS)ₙ (SEQ ID NO: 143), SGSETPGTSESATPES (SEQ ID NO: 135), or (XP)ₙ (SEQ ID NO: 144) motif, or a combination of any of these, wherein n is independently an integer between 1 and 30, and wherein Xis any amino acid. In some embodiments, n is 1, 2, 3, 4, 5, 6, 7, 8, 9, 10, 11, 12, 13, 14, or 15. In some embodiments, a linker comprises SGSETPGTSESATPES (SEQ ID NO: 135), and SGGS (SEQ ID NO: 137). In some embodiments, a linker comprises SGGSSGSETPGTSESATPESSGGS (SEQ ID NO: 145). In some embodiments, a linker comprises SGGSSGGSSGSETPGTSESATPESSGGSSGGS (SEQ ID NO: 146). In some embodiments, a linker comprises GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS (SEQ ID NO: 147). In some embodiments, the linker is 24 amino acids in length. In some embodiments, the linker comprises the amino acid sequence SGGSSGGSSGSETPGTSESATPES (SEQ ID NO: 148). In certain embodiments of the disclosed base editors, the linker comprises a sequence that has at least 90%, at least 95%, at least 98%, or at least 99% identity to the sequence of SEQ ID NO: 148.

In some embodiments, the linker is 40 amino acids in length. In some embodiments, the linker comprises the amino acid sequence SGGSSGGSSGSETPGTSESATPESSGGSSGGSSGGSSGGS (SEQ ID NO: 149). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGS SGGS (SEQ ID NO: 150). In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSAP GTSTEPSEGSAPGTSESATPESGPGSEPATS (SEQ ID NO: 151). It should be appreciated that any of the linkers provided herein may be used to link a first adenosine deaminase and a second adenosine deaminase; an adenosine deaminase (e.g., a first or a second adenosine deaminase) and a napDNAbp; a napDNAbp and an NLS; or an adenosine deaminase (e.g., a first or a second adenosine deaminase) and an NLS.

In some embodiments, any of the base editors described herein, comprise an adenosine deaminase and a napDNAbp that are fused to each other via a linker. In some embodiments, any of the base editors described herein, comprise a first adenosine deaminase and a second adenosine deaminase that are fused to each other via a linker. In some embodiments, any of the base editors described herein, comprise an NLS, which may be fused to an adenosine deaminase (e.g., a first and/or a second adenosine deaminase), a nucleic acid programmable DNA binding protein (napDNAbp. Various linker lengths and flexibilities between an adenosine deaminase (e.g., an engineered ecTadA) and a napDNAbp (e.g., a Cas9 domain), and/or between a first adenosine deaminase and a second adenosine deaminase can be employed (e.g., ranging from very flexible linkers of the form (GGGGS)ₙ (SEQ ID NO: 138), (GGGGS)ₙ (SEQ ID NO: 139), and (G)ₙ (SEQ ID NO: 140) to more rigid linkers of the form (EAAAK)ₙ (SEQ ID NO: 141), (SGGS)ₙ (SEQ ID NO: 137), SGSETPGTSESATPES (SEQ ID NO: 135) (see, e.g., Guilinger J P, Thompson D B, Liu D R. Fusion of catalytically inactive Cas9 to Fokl nuclease improves the specificity of genome modification. Nat. Biotechnol. 2014; 32(6): 577-82; the entire contents are incorporated herein by reference) and (XP). (SEQ ID NO: 144)) in order to achieve the optimal length for deaminase activity for the specific application. In some embodiments, n is 1, 2, 3, 4, 5, 6, 7, 8, 9, 10, 11, 12, 13, 14, or 15. In some embodiments, the linker comprises a (GGS)ₙ (SEQ ID NO: 152) motif, wherein n is 1, 3, or 7. In some embodiments, the adenosine deaminase and the napDNAbp, and/or the first adenosine deaminase and the second adenosine deaminase of any of the base editors described herein are fused via a linker comprising the amino acid sequence SGSETPGTSESATPES (SEQ ID NO: 135), SGGS (SEQ ID NO: 137), SGGSSGSETPGTSESATPESSGGS (SEQ ID NO: 145), SGGSSGGSSGSETPGTSESATPESSGGSSGGS (SEQ ID NO: 146), or GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS (SEQ ID NO: 147). In some embodiments, the linker is 24 amino acids in length. In some embodiments, the linker comprises the amino acid sequence SGGSSGGSSGSETPGTSESATPES (SEQ ID NO: 148). In some embodiments, the linker is 32 amino acids in length. In some embodiments, the linker is 32 amino acids in length. In some embodiments, the linker comprises the amino acid sequence (SGGS)₂-SGSETPGTSESATPES-(SGGS)₂ (SEQ ID NO: 136), which may also be referred to as (SGGS)₂-XTEN-(SGGS)₂ (SEQ ID NO: 136). In some embodiments, the linker comprises the amino acid sequence, wherein n is 0, 1, 2, 3, 4, 5, 6, 7, 8, 9, or 10. In some embodiments, the linker is 40 amino acids in length. In some embodiments, the linker comprises the amino acid sequence SGGSSGGSSGSETPGTSESATPESSGGSSGGSSGGSSGGS (SEQ ID NO: 149). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGS SGGS (SEQ ID NO: 150). In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSAP GTSTEPSEGSAPGTSESATPESGPGSEPATS (SEQ ID NO: 151).

### (2) UGI Domain

In other embodiments, the base editors described herein may comprise one or more uracil glycosylase inhibitors. The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 336. In some embodiments, the UGI proteins described herein include fragments of UGI and proteins homologous to a UGI or a UGI fragment. For example, in some embodiments, a UGI domain comprises a fragment of the amino acid sequence set forth in SEQ ID NO: 336. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 336. In some embodiments, a UGI comprises an amino acid sequence homologous to the amino acid sequence set forth in SEQ ID NO: 336, or an amino acid sequence homologous to a fragment of the amino acid sequence set forth in SEQ ID NO: 336. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 336. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 336. In some embodiments, the UGI comprises the following amino acid sequence:

### Uracil-DNA Glycosylase Inhibitor:

> splP14739|UNGI_BPPB2
(SEQ ID NO: 336)
MTNLSDIIEKETGKQLVIQESILMLPEEVEEVIGN
KPESDILVHTAYDESTDENVMLLTSDAPEYKPWAL
VIQDSNGENKIKML.

The base editors described herein may comprise more than one UGI domain, which may be separated by one or more linkers as described herein. It will also be understood that in the context of the herein disclosed base editors, the UGI domain may be linked to a deaminase domain or

### (3) NLS Domains

In some embodiments, the base editors described herein further comprise one or more nuclear targeting sequences, for example, a nuclear localization sequence (NLS). In some embodiments, a NLS comprises an amino acid sequence that facilitates the importation of a protein, that comprises an NLS, into the cell nucleus (e.g., by nuclear transport). In some embodiments, any of the base editors described herein further comprise one or more nuclear localization sequences (NLSs). In certain embodiments, any of the base editors comprise two NLSs. In some embodiments, one or more of the NLSs are bipartite NLSs (“bpNLS”). In certain embodiments, the disclosed base editors comprise two bipartite NLSs. In some embodiments, the disclosed base editors comprise more than two bipartite NLSs. In particular embodiments, the one or more NLSs of the disclosed base editors comprise the amino acid sequence of MKRTADGSEFESPKKKRKV (SEQ ID NO: 343). In some embodiments, the one or more NLSs of the disclosed base editors comprise an amino acid sequence that differs by 1, 2, 3, or 4 amino acids from the sequence of SEQ ID NO: 343.

In some embodiments, the NLS is fused to the N-terminus of the base editor. In some embodiments, the NLS is fused to the C-terminus of the base editor. In some embodiments, the NLS is fused to the C-terminus of the napDNAbp. In some embodiments, the NLS is fused to the N-terminus of the adenosine deaminase. In some embodiments, the NLS is fused to the C-terminus of the adenosine deaminase. In some embodiments, the NLS is fused to the base editor via one or more linkers. In some embodiments, the NLS is fused to the base editor without a linker.

In some embodiments, the base editors described herein do not comprise a linker. In some embodiments, a linker is present between one or more of the domains or proteins (e.g., adenosine deaminase, napDNAbp, and/or NLS). In some embodiments, the “]-[” used in the general architecture above indicates the presence of an optional linker.

In some embodiments, the general architecture of exemplary base editors with a first adenosine deaminase, a second adenosine deaminase, and a napDNAbp comprises any one of the following structures, where NLS is a nuclear localization sequence (e.g., any NLS provided herein), NH₂ is the N-terminus of the base editor, and COOH is the C-terminus of the base editor.

In some embodiments, the NLS comprises an amino acid sequence of any one of the NLS sequences provided or referenced herein. Such sequences are well-known in the art and can include the following examples:

| | | SEQUENCE |
|---|---|---|
| DESCRIPTION | SEQUENCE | IDENTIFIER |
| NLS OF SV40 | PKKKRKV | 337 |
| LARGE T-AG | | |
| NLS OF | VSRKRPRP | 338 |
| POLYOMA | | |
| LARGE T-AG | | |
| NLS OF C- | PAAKRVKLD | 339 |
| MYC | | |
| NLS OF TUS- | KLKIKRPVK | 340 |
| PROTEIN | | |
| NLS OF | EGAPPAKRAR | 341 |
| HEPATITIS D | | |
| VIRUS | | |
| ANTIGEN | | |
| NLS OF | PPQPKKKPLDGE | 342 |
| MURINE P53 | | |
| NLS | MKRTADGSEFESPKKKRKV | 343 |
| NLS OF | AVKRPAATKKAGQAKKKKLD | 344 |
| NUCLEOPLASM | | |
| IN | | |
| NLS | SGGSKRTADGSEFEPKKKRKV | 345 |
| NLS OF EGL- | MSRRRKANPTKLSENAKKLAK | 346 |
| 13 | EVEN | |
| NLS | KRTADGSEFEPKKKRKV | 348 |
| NLS | MDSLLMNRRKFLYQFKN | 347 |
| | VRWAKGRRETYLC | |

In other embodiments, the NLS comprises the amino acid sequence: NLSKRPAAIKKAGQAKKKK (SEQ ID NO: 158), PAAKRVKLD (SEQ ID NO: 339), RQRRNELKRSF (SEQ ID NO: 159), or NQSSNFGPMKGGNFGGRSSGPYGGGGQYFAKPRNQGGY (SEQ ID NO: 160). In exemplary embodiments, the NLS comprises the amino acid sequence of PKKKRKV (SEQ ID NO: 337) or MDSLLMNRRKFLYQFKNVRWAKGRRETYLC (SEQ ID NO: 347).

A representative nuclear localization signal is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization signals often comprise proline residues. A variety of nuclear localization signals have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated herein by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 337)); (ii) a bipartite motif consisting of two basic domains separated by a variable number of spacer amino acids and exemplified by the Xenopus nucleoplasmin NLS (KRXXXXXXXXXXKKKL (SEQ ID NO: 161)), wherein X is any amino acid; and (iii) noncanonical sequences such as M9 of the hnRNP Al protein, the influenza virus nucleoprotein NLS, and the yeast Gal4 protein NLS (Dingwall and Laskey, Trends Biochem Sci. 1991 Dec; 16(12):478-81).

Nuclear localization signals appear at various points in the amino acid sequences of proteins. NLSs have been identified at the N-terminus, the C-terminus, and in the central region of proteins. Thus, the specification provides base editors that may be modified with one or more NLSs at the C-terminus, the N-terminus, as well as at an internal region of the base editor. The residues of a longer sequence that do not function as component NLS residues should be selected so as not to interfere, for example tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a base editor (or base editor) to include one or more NLSs. In one aspect, the base editors can be engineered to express a base editor that is translationally fused at its N-terminus or its C-terminus (or both) to one or more NLSs, i.e., to form a base editor-NLS fusion construct. In other embodiments, the base editor-encoding nucleotide sequence can be genetically modified to incorporate a reading frame that encodes one or more NLSs in an internal region of the encoded base editor. In addition, the NLSs may include various amino acid linkers or spacer regions encoded between the base editor and the N-terminally, C-terminally, or internally-attached NLS amino acid sequence. Thus, the present disclosure also provides for nucleotide constructs, vectors, and host cells for expressing base editors that comprise a base editor and one or more NLSs.

The base editors described herein may also comprise nuclear localization signals which are linked to a base editor through one or more linkers, e.g., polymeric, peptidic, amino acid, polysaccharide, chemical, or nucleic acid linker element. In certain embodiments, the NLS is linked to a base editor using an XTEN linker, as set forth in SEQ ID NO: 148. The linkers within the contemplated scope of the disclosure are not intented to have any limitations and can be any suitable type of molecule (e.g., polymer, amino acid, polysaccharide, nucleic acid, lipid, or any synthetic chemical linker domain) and be joined to the base editor by any suitable strategy that effectuates forming a bond (e.g., covalent linkage, hydrogen bonding) between the base editor and the one or more NLSs.

The NLS examples above are non-limiting. The disclosed base editors may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which are incorporated herein by reference.

In some embodiments, the base editors described herein do not comprise a linker. In some embodiments, a linker is present between one or more of the domains or proteins (e.g., first adenosine deaminase, second adenosine deaminase, napDNAbp, and/or NLS). In some embodiments, the “]-[” used in the general architecture above indicates the presence of an optional linker.

### (4) Split-Intein/Intein Domains

It will be understood that in some embodiments (e.g., delivery of a base editor in vivo using AAV particles), it may be advantageous to split a polynucleotide encoding a base editor into an N-terminal half and a C-terminal half, deliver N- and C-terminal halves separately, and then allow their colocalization to reform the complete protein (or base editor as the case may be) within the cell. Separate halves of a protein or a base editor may each comprise a split-intein tag to facilitate the reformation of the complete protein or base editor by the mechanism of protein trans splicing.

Protein trans-splicing, catalyzed by split inteins, provides an entirely enzymatic method for protein ligation. A split-intein is essentially a contiguous intein (e.g. a mini-intein) split into two pieces named N-intein and C-intein, respectively. The N-intein and C-intein of a split intein can associate non-covalently to form an active intein and catalyze the splicing reaction essentially in same way as a contiguous intein does. Split inteins have been found in nature and also engineered in laboratories. As used herein, the term “split intein” refers to any intein in which one or more peptide bond breaks exists between the N-terminal and C-terminal amino acid sequences such that the N-terminal and C-terminal sequences become separate molecules that can non-covalently reassociate, or reconstitute, into an intein that is functional for trans-splicing reactions. Any catalytically active intein, or fragment thereof, may be used to derive a split intein for use in the methods of the invention. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing trans-splicing reactions.

In some aspects of the present disclosure, any of the disclosed base editors are formulated for delivery as two halves. In these embodiments, a composition comprising two polynucleotides (or two vectors) is provided, where the first polynucleotide encodes the N-terminal half of the base editor fused to an N-intein, and the second polynucleotide encodes the C-terminal half of the base editor fused to an C-intein. In some embodiments, the base editor inteins are divided, or “split,” at a site in the napDNAbp domain of the base editor. In some embodiments, the base editor inteins are divided at an amino acid residue within five or fewer positions of residue 574 of the napDNAbp domain. In other embodiments, the base editor inteins are divided at an amino acid residue within give or fewer positions of residue 638 of the napDNAbp domain. In particular embodiments, the base editor inteins are divided at residue 574 or residue 638 of the napDNAbp domain (e.g., an SpCas9 domain). In particular embodiments, each of two split intein halves are fused to each half of a polynucleotide encoding a base editor (e.g., ABE8e, BE4max, or BE4max-VRQR) within the S. pyogenes Cas9 nickase domain (SEQ ID NO: 107) of the base editor immediately before Cys 574 or before Thr 638 (i.e., immediately after residue 573 or after residue 637).

Exemplary inteins of the disclosure include the Nostoc punctiforme (Npu) intein system. This synthetic intein based on the dnaE intein has been described (see Zettler, J., Schutz, V. & Mootz, H. D., FEBS letters 583, 909-914 (2009), incorporated herein by reference). Accordingly, in particular embodiments, the disclosure provides compositions of a vector encoding a base editor wherein each split DnaE intein half from Nostoc punctiforme (Npu) is fused to each half of a polynucleotide encoding a base editor (e.g., ABE8e, BE4max, or BE4max-VRQR) within the S. pyogenes Cas9 nickase domain (SEQ ID NO: 107) of the base editor immediately before Cys 574 (or immediately after residue 573). Reference is made to Levy et al., Nat Biomed Eng. 2020 Jan; 4(1):97-110.

In particular embodiments, the disclosure provides compositions of vectors wherein the N-terminal portion of the base editor comprises a portion of any one of SEQ ID NOs: 350-388 that corresponds to amino acids 1-573 of the napDNAbp domain of the base editor. In some embodiments, the disclosure provides compositions of vectors wherein the C-terminal portion of the Cas9 protein comprises a portion of any one of SEQ ID NOs: 350-388 that corresponds to amino acids 574-1368 of the napDNAbp domain. In particular embodiments, the disclosure provides compositions of vectors wherein the N-terminal portion of the base editor comprises a portion of any one of SEQ ID NOs: 350-388 that corresponds to amino acids 1-573 of the Cas9 domain of the base editor and wherein the C-terminal portion of the same base editor comprises a portion of any one of SEQ ID NOs: 350-388 that corresponds to amino acids 574-1368 of the Cas9 domain. In some embodiments, the base editor comprises any of the sequences of SEQ ID NOs: 350 (BE4max), 372 (BE4max-VRQR), 378 (ABE7.10), and 379 (ABE8e).

Another exemplary split intein is the Ssp DnaE intein, which comprises two subunits, namely, DnaE-N and DnaE-C. The two different subunits are encoded by separate genes, namely dnaE-n and dnaE-c, which encode the DnaE-N and DnaE-C subunits, respectively. DnaE is a naturally occurring split intein in Synechocytis sp. PCC6803 and is capable of directing trans-splicing of two separate proteins, each comprising a fusion with either DnaE-N or DnaE-C. Non-limiting examples of intein pairs that may be used in accordance with the present disclosure include: Cfa DnaE intein, Npu DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference).

As used herein, the “N-terminal split intein (In)” refers to any intein sequence that comprises an N-terminal amino acid sequence that is functional for trans-splicing reactions. An In thus also comprises a sequence that is spliced out when trans-splicing occurs. An In can comprise a sequence that is a modification of the N-terminal portion of a naturally occurring intein sequence. For example, an In can comprise additional amino acid residues and/or mutated residues so long as the inclusion of such additional and/or mutated residues does not render the In non-functional in trans-splicing. Preferably, the inclusion of the additional and/or mutated residues improves or enhances the trans-splicing activity of the In.

As used herein, the “C-terminal split intein (Ic)” refers to any intein sequence that comprises a C-terminal amino acid sequence that is functional for trans-splicing reactions. In one aspect, the Ic comprises 4 to 7 contiguous amino acid residues, at least 4 amino acids of which are from the last (3-strand of the intein from which it was derived. An Ic thus also comprises a sequence that is spliced out when trans-splicing occurs. An Ic can comprise a sequence that is a modification of the C-terminal portion of a naturally occurring intein sequence. For example, an Ic can comprise additional amino acid residues and/or mutated residues so long as the inclusion of such additional and/or mutated residues does not render the In non-functional in trans-splicing. Preferably, the inclusion of the additional and/or mutated residues improves or enhances the trans-splicing activity of the Ic.

Inteins intein-N and intein-C may be fused to the N-terminal portion of the split base editor and the C-terminal portion of the split base editor, respectively, for the joining of the N-terminal portion of the split base editor and the C-terminal portion of the split base editor. For example, in some embodiments, an intein-N is fused to the C-terminus of the N-terminal portion of the split base editor, i.e., to form a structure of N-[N-terminal portion of the split base editor]-[intein-N]-C. In some embodiments, an intein-C is fused to the N-terminus of the C-terminal portion, i.e., to form a structure of N-[intein-C]-[C-terminal portion of the split base editor]-C. The mechanism of intein-mediated protein splicing for joining the proteins the inteins are fused to (e.g., split base editor) is known in the art, e.g., as described in Shah et al., Chem Sci. 2014; 5(1):446-461, incorporated herein by reference.

Split inteins may be created from contiguous inteins by engineering one or more split sites in the unstructured loop or intervening amino acid sequence between the −12 conserved beta-strands found in the structure of mini-inteins. Some flexibility in the position of the split site within regions between the beta-strands may exist, provided that creation of the split will not disrupt the structure of the intein, the structured beta-strands in particular, to a sufficient degree that protein splicing activity is lost.

In protein trans-splicing, one precursor protein consists of an N-extein part followed by the N-intein, another precursor protein consists of the C-intein followed by a C-extein part, and a trans-splicing reaction (catalyzed by the N- and C-inteins together) excises the two intein sequences and links the two extein sequences with a peptide bond. Protein trans-splicing, being an enzymatic reaction, can work with very low (e.g., micromolar) concentrations of proteins and can be carried out under physiological conditions.

Exemplary intein sequences are as follows:

| NAME | SEQUENCE OF INTEIN |
|---|---|
| NPU DNAE N- | CLSYETEILTVEYGLLPIGKIVEKRIECTVYSVDN |
| TERMINAL | NGNIYTQPVAQWHDRGEQEVFEYCLEDGSLIRATK |
| PROTEIN | DHKFMTVDGQMLPIDEIFERELDLMRVDNLPN |
| | (SEQ ID NO: 389) |
| NPU DNAE C- | MIKIATRKYLGKQNVYDIGVERDHNFALKNG |
| TERMINAL | FIASN |
| PROTEIN | (SEQ ID NO: 390) |
| CFA-N | CLSYDTEILTVEYGFLPIGKIVEERIECTVYTVDK |
| PROTEIN: | NGFVYTQPIAQWHNRGEQEVFEYCLEDGSIIRATK |
| | DHKFMTTDGQMLPIDEIFERGLDLKQVDGLP |
| | (SEQ ID NO: 391) |
| CFA-C | MKRTADGSEFESPKKKRKVKIISRKSLGTQNVYDI |
| PROTEIN: | GVEKDHNFLLKNGLVASN (SEQ ID NO: 392) |

Although inteins are most frequently found as a contiguous domain, some exist in a naturally split form. In this case, the two fragments are expressed as separate polypeptides and must associate before splicing takes place, so-called protein trans-splicing.

Additional naturally occurring or engineered split-intein sequences are known in the or can be made from whole-intein sequences described herein or those available in the art. Examples of split-intein sequences can be found in Stevens et al., “A promiscuous split intein with expanded protein engineering applications,” PNAS, 2017, Vol.114: 8538-8543; Iwai et al., Highly efficient protein trans-splicing by a naturally split DnaE intein from Nostc punctiforme, FEBS Lett, 580: 1853-1858, each of which are incorporated herein by reference. Additional split intein sequences can be found, for example, in WO 2013/045632, WO 2014/055782, WO 2016/069774, and EP2877490, the contents each of which are incorporated herein by reference.

In addition, protein splicing in trans has been described in vivo and in vitro (Shingledecker, et al., Gene 207:187 (1998), Southworth, et al., EMBO J. 17:918 (1998); Mills, et al., Proc. Natl. Acad. Sci. USA, 95:3543-3548 (1998); Lew, et al., J. Biol. Chem., 273:15887-15890 (1998); Wu, et al., Biochim. Biophys. Acta 35732:1 (1998b), Yamazaki, et al., J. Am. Chem. Soc. 120:5591 (1998), Evans, et al., J. Biol. Chem. 275:9091 (2000); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999)) and provides the opportunity to express a protein as to two inactive fragments that subsequently undergo ligation to form a functional product.

### Exemplary Base Editors

In various aspects, the instant specification provides base editors and methods of using the same to treat Usher syndrome . In particular, the inventors surprisingly found that adenosine base editors could be used to efficiently mutate specific residues of the USH2A gene both in vitro and in vivo, which is useful for the treatment of certain Usher syndrome and retinitis pigmentosa.

In certain aspects, methods provided herein utilize base editors (e.g., adenosine base editors) known in the art in order to make one or more desired nucleic acid modifications. The state of the art has described numerous base editors as of this filing. The methods and approaches herein described may be applied to any previously known base editor, or to base editors that may be developed in the future. Exemplary base editors that may be used in accordance with the present disclosure include those described in the following references and/or patent publications, each of which are incorporated by reference in their entireties: in Rees & Liu, Nat Rev Genet. 2018; 19(12):770-788 and Koblan et al., Nat Biotechnol. 2018; 36(9):843-846; as well as.U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; International Publication No. WO 2019/023680, published Jan. 31, 2019; International Application No. PCT/US2019/033848, filed May 23, 2019, which published as Publication No. WO 2019/226593 on Nov. 28, 2019; International Publication No. WO 2018/0176009, published Sep. 27, 2018, International Publication No. WO 2020/041751, published Feb. 27, 2020; International Publication No. WO 2020/051360, published Mar. 12, 2020; International Patent Publication No. WO 2020/102659, published May 22, 2020; International Publication No. WO 2020/086908, published Apr. 30, 2020; International Publication No. WO 2020/181180, published Sep. 10, 2020; International Publication No. WO 2020/214842, published Oct. 22, 2020; International Publication No. WO 2020/092453, published May 7, 2020; International Publication No. WO2020/236982, published Nov. 26, 2020; International Application No. PCT/US2020/624628, filed Nov. 25, 2020, and International Application No. PCT/US2021/016827, filed Feb. 5, 2021.

In various aspects, the improved or modified base editors described herein have the following generalized structure:

A-B-C,

wherein “A” is a Cas moiety or napDNA/RNAbp, “B” is nucleic acid effector domain (e.g., an adenosine deaminase), and “C” is one or more nuclear localization signals (NLS). In addition, the “-” represents a linker that covalently joins moieties A, B, and C. The linkers can be any suitable type (e.g., amino acid sequences or other biopolymers, or synthetic chemical linkages in the case where the moieties are bioconjugated to one another) or length. In addition, a functional base editor of the invention would also include one or more “R” or guide sequences (e.g., guide RNA in the case of a Cas9 or Cas9 equivalent) in order to carry out the DNA/RNA-programmable functionality of base editors for targeting specific sites to be corrected.

The order of linkage of the moieties is not meant to be particularly limiting so long as the particular arrangement of the elements of moieties produces a functional base editor. That is, the improved base editors of the invention may also include editors represented by the following structures:

B-A-C;

B-C-A;

C-B-A;

C-A-B; and

A-C-B.

In some embodiments, the base editors described herein can be made as a recombinant base editor comprising one or more protein domains, thereby generating a base editor. In certain embodiments, the base editors described herein comprise one or more features that improve the base editing activity (e.g., efficiency, selectivity, and/or specificity) of the base editor proteins. For example, the base editors described herein may comprise a Cas9 domain that has reduced nuclease activity. In some embodiments, the base editors described herein may have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-deaminated) strand containing a T opposite the targeted A. Mutation of the catalytic residue (e.g., D10 to A10) of Cas9 prevents cleavage of the edited strand containing the targeted A residue. Such Cas9 variants are able to generate a single-strand DNA break (nick) at a specific location based on the gRNA-defined target sequence, leading to repair of the non-edited strand, ultimately resulting in a T to C change on the non-edited strand.

In particular, the disclosure provides adenosine base editors that can be used to disrupt a splice site or introduce a point mutation in the human USH2A gene. Exemplary domains used in base editors, including adenosine deaminases, napDNA/RNAbp (e.g., Cas9), and nuclear localization sequences (NLSs) are described in further detail below. Some aspects of the disclosure provide base editors comprising a nucleic acid programmable DNA binding protein (napDNAbp) and a deaminase (e.g., adenosine or cytidine deaminase). In some embodiments, any of the base editors described herein is a base editor. In some embodiments, the napDNAbp is a Cas9 domain, a Cpf 1 domain, a CasX domain, a CasY domain, a C2c1 domain, a C2c2 domain, aC2c3 domain, or an Argonaute domain. In some embodiments, the napDNAbp is any napDNAbp described herein. Some aspects of the disclosure provide base editors comprising a Cas9 domain and a deaminase (e.g., an adenosine or cytidine deaminase). The Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the deaminases (e.g., adenosine or cytidine deaminases) provided herein. In some embodiments, the base editor comprises the structure:

NH₂-[deaminase]-[napDNAbp]-COOH; or

NH₂-[napDNAbp][deaminase]-COOH

In some embodiments, the base editors comprising an adenosine deaminase and a napDNAbp (e.g., Cas9 domain) do not include a linker sequence. In some embodiments, a linker is present between the adenosine deaminase domain and the napDNAbp. In some embodiments, the “-” used in the general architecture above indicates the presence of an optional linker. In some embodiments, the adenosine deaminase and the napDNAbp are fused via any of the linkers provided herein. For example, in some embodiments the adenosine deaminase and the napDNAbp are fused via any of the linkers provided above in the section entitled “Linkers”. In some embodiments, the adenosine deaminase and the napDNAbp are fused via a linker that comprises between 1 and 200 amino acids. In some embodiments, the adenosine deaminase and the napDNAbp are fused via a linker that comprises from 1 to 5, 1 to 10, 1 to 20, 1 to 30, 1 to 40, 1 to 50, 1 to 60, 1 to 80, 1 to 100, 1 to 150, 1 to 200, 5 to 10, 5 to 20, 5 to 30, 5 to 40, 5 to 60, 5 to 80, 5 to 100, 5 to 150, 5 to 200, 10 to 20, 10 to 30, 10 to 40, 10 to 50, 10 to 60, 10 to 80, 10 to 100, 10 to 150, 10 to 200, 20 to 30, 20 to 40, 20 to 50, 20 to 60, 20 to 80, 20 to 100, 20 to 150, 20 to 200, 30 to 40, 30 to 50, 30 to 60, 30 to 80, 30 to 100, 30 to 150, 30 to 200, 40 to 50, 40 to 60, 40 to 80, 40 to 100, 40 to 150, 40 to 200, 50 to 60 50 to 80, 50 to 100, 50 to 150, 50 to 200, 60 to 80, 60 to 100, 60 to 150, 60 to 200, 80 to 100, 80 to 150, 80 to 200, 100 to 150, 100 to 200, or 150 to 200 amino acids in length. In some embodiments, the adenosine deaminase and the napDNAbp are fused via a linker that comprises 3, 4, 16, 24, 32, 64, 100, or 104 amino acids in length. In some embodiments, the adenosine deaminase and the napDNAbp are fused via a linker that comprises the amino acid sequence of SGSETPGTSESATPES (SEQ ID NO: 135), SGGS (SEQ ID NO: 137), SGGSSGSETPGTSESATPESSGGS (SEQ ID NO: 145), SGGSSGGSSGSETPGTSESATPESSGGSSGGS (SEQ ID NO: 146), or GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS (SEQ ID NO: 147). In some embodiments, the adenosine deaminase and the napDNAbp are fused via a linker comprising the amino acid sequence SGSETPGTSESATPES (SEQ ID NO: 135), which may also be referred to as the XTEN linker. In some embodiments, the linker is 24 amino acids in length. In some embodiments, the linker comprises the amino acid sequence SGGSSGGSSGSETPGTSESATPES (SEQ ID NO: 148). In some embodiments, the linker is 32 amino acids in length. In some embodiments, the linker comprises the amino acid sequence (SGGS)₂-SGSETPGTSESATPES-(SGGS)₂ (SEQ ID NO: 136), which may also be referred to as (SGGS)₂-XTEN-(SGGS)₂ (SEQ ID NO: 136). In some embodiments, the linker comprises the amino acid sequence (SGGS).-SGSETPGTSESATPES-(SGGS), (SEQ ID NO: 142), wherein n is 0, 1, 2, 3, 4, 5, 6, 7, 8, 9, or 10. In some embodiments, the linker is 40 amino acids in length. In some embodiments, the linker comprises the amino acid sequence SGGSSGGSSGSETPGTSESATPESSGGSSGGSSGGSSGGS (SEQ ID NO: 149). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGS SGGS (SEQ ID NO: 150). In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSAP GTSTEPSEGSAPGTSESATPESGPGSEPATS (SEQ ID NO: 151).

In other embodiments, the base editors comprising an cytidine deaminase and a napDNAbp (e.g., Cas9 domain) do not include a linker sequence. In some embodiments, a linker is present between the cytidine deaminase domain and the napDNAbp. In some embodiments, the “-” used in the general architecture above indicates the presence of an optional linker. In some embodiments, the cytidine deaminase and the napDNAbp are fused via any of the linkers provided herein. For example, in some embodiments the cytidine deaminase and the napDNAbp are fused via any of the linkers provided above in the section entitled “Linkers”. In some embodiments, the cytidine deaminase and the napDNAbp are fused via a linker that comprises between 1 and 200 amino acids. In some embodiments, the cytidine deaminase and the napDNAbp are fused via a linker that comprises from 1 to 5, 1 to 10, 1 to 20, 1 to 30, 1 to 40, 1 to 50, 1 to 60, 1 to 80, 1 to 100, 1 to 150, 1 to 200, 5 to 10, 5 to 20, 5 to 30, 5 to 40, 5 to 60, 5 to 80, 5 to 100, 5 to 150, 5 to 200, 10 to 20, 10 to 30, 10 to 40, 10 to 50, 10 to 60, 10 to 80, 10 to 100, 10 to 150, 10 to 200, 20 to 30, 20 to 40, 20 to 50, 20 to 60, 20 to 80, 20 to 100, 20 to 150, 20 to 200, 30 to 40, 30 to 50, 30 to 60, 30 to 80, 30 to 100, 30 to 150, 30 to 200, 40 to 50, 40 to 60, 40 to 80, 40 to 100, 40 to 150, 40 to 200, 50 to 60 50 to 80, 50 to 100, 50 to 150, 50 to 200, 60 to 80, 60 to 100, 60 to 150, 60 to 200, 80 to 100, 80 to 150, 80 to 200, 100 to 150, 100 to 200, or 150 to 200 amino acids in length.

In some embodiments, the base editors described herein further comprise one or more nuclear targeting sequences, for example, a nuclear localization sequence (NLS). In some embodiments, a NLS comprises an amino acid sequence that facilitates the importation of a protein, that comprises an NLS, into the cell nucleus (e.g., by nuclear transport). In some embodiments, any of the base editors described herein further comprise a nuclear localization sequence (NLS). In some embodiments, the NLS is fused to the N-terminus of the base editor. In some embodiments, the NLS is fused to the C-terminus of the base editor. In some embodiments, the NLS is fused to the N-terminus of the napDNAbp. In some embodiments, the NLS is fused to the C-terminus of the napDNAbp. In some embodiments, the NLS is fused to the N-terminus of the adenosine deaminase. In some embodiments, the NLS is fused to the C-terminus of the adenosine deaminase. In some embodiments, the NLS is fused to the base editor via one or more linkers. In some embodiments, the NLS is fused to the base editor without a linker. In some embodiments, the NLS comprises an amino acid sequence of any one of the NLS sequences provided or referenced herein. In some embodiments, the NLS comprises an amino acid sequence as set forth in any one of SEQ ID NOs: 30-33. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the general architecture of exemplary base editors with an adenosine deaminase and a napDNAbp comprises any one of the following structures, where NLS is a nuclear localization sequence (e.g., any NLS provided herein), NH₂ is the N-terminus of the base editor, and COOH is the C-terminus of the base editor. Base editors comprising an adenosine deaminase, a napDNAbp, and a NLS:

NH₂-[NLS]-[deaminase]-[napDNAbp]-COOH;

NH₂-[deaminase]-[NLS]-[napDNAbp]-COOH;

NH₂-[deaminase]-[napDNAbp]-[NLS]-COOH;

NH₂-[NLS]-[napDNAbp]-[deaminase]-COOH;

NH₂-[napDNAbp]-[NLS]-[deaminase]-COOH; and

NH₂-[napDNAbp][deaminase]-[NLS]-COOH.

In some embodiments, the base editors described herein do not comprise a linker. In some embodiments, a linker is present between one or more of the domains or proteins (e.g., cytidine deaminase or adenosine deaminase domain, napDNAbp domain, and/or NLS). In some embodiments, the “]-[” used in the general architecture above indicates the presence of an optional linker.

In certain embodiments, the disclosed adenine base editors comprise an ABE7.10 or ABE8 architecture, which comprises NH₂-[NLS]-[first adenosine deaminase]-[second adenosine deaminase]-[napDNAbp]-[NLS]-COOH. In certain embodiments, the disclosed adenine base editors comprise an ABE8 monomer architecture, which comprises NH₂-[NLS]-[adenosine deaminase]-[napDNAbp]-[NLS]-COOH. In SOME embodiments, the disclosed cytidine base editors comprise an BE4 architecture, which comprises NH₂-[NLS]-[cytidine deaminase]-[napDNAbp]-[NLS]-COOH.

In some embodiments, the base editors described herein further comprise one or more nuclear targeting sequences, for example, a nuclear localization sequence (NLS). In some embodiments, a NLS comprises an amino acid sequence that facilitates the importation of a protein, that comprises an NLS, into the cell nucleus (e.g., by nuclear transport). In some embodiments, any of the base editors described herein further comprise one or more nuclear localization sequences (NLSs). In certain embodiments, any of the base editors comprise two NLSs. In some embodiments, one or more of the NLSs are bipartite NLSs (“bpNLS”). In certain embodiments, the disclosed base editors comprise two bipartite NLSs. In some embodiments, the disclosed base editors comprise more than two bipartite NLSs.

In some embodiments, the NLS is fused to the N-terminus of the base editor. In some embodiments, the NLS is fused to the C-terminus of the base editor. In some embodiments, the NLS is fused to the C-terminus of the napDNAbp. In some embodiments, the NLS is fused to the N-terminus of the adenosine deaminase. In some embodiments, the NLS is fused to the C-terminus of the adenosine deaminase. In some embodiments, the NLS is fused to the base editor via one or more linkers. In some embodiments, the NLS is fused to the base editor without a linker.

In some embodiments, the NLS comprises an amino acid sequence of any one of the NLS sequences provided or referenced herein. In some embodiments, the NLS comprises an amino acid sequence as set forth in SEQ ID NO: 337 or SEQ ID NO: 347.

The base editors described herein also may include one or more additional elements. In certain embodiments, an additional element may comprise an effector of base repair, such as an inhibitor of base repair.

In some embodiments, the base editors described herein may comprise one or more heterologous protein domains (e.g., about or more than about 1, 2, 3, 4, 5, 6, 7, 8, 9, 10, or more domains in addition to the base editors components). A base editor may comprise any additional protein sequence, and optionally a linker sequence between any two domains. Other exemplary features that may be present are localization sequences, such as cytoplasmic localization sequences, export sequences, such as nuclear export sequences, or other localization sequences, as well as sequence tags.

Examples of heterologous protein domains that may be fused to a base editor or component thereof (e.g., the napDNAbp domain, the nucleotide modification domain, or the NLS domain) include, without limitation, epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A base editor may be fused to a gene sequence encoding a protein or a fragment of a protein that binds DNA molecules or binds other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a base editor are described in US Patent Publication No. 2011/0059502, published Mar. 10, 2011, and incorporated herein by reference in its entirety.

In an aspect of the disclosure, a reporter gene which includes, but is not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In certain embodiments of the disclosure the gene product is luciferase. In a further embodiment of the disclosure the expression of the gene product is decreased.

Other exemplary features that may be present are tags that are useful for solubilization, purification, or detection of the base editor. Suitable protein tags provided herein include, but are not limited to, biotin carboxylase carrier protein (BCCP) tags, myc-tags, calmodulin-tags, FLAG-tags, hemagglutinin (HA)-tags, bgh-PolyA tags, polyhistidine tags, and also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags , biotin ligase tags, FlAsH tags, V5 tags, and SBP-tags. Additional suitable sequences will be apparent to those of skill in the art. In some embodiments, the base editor comprises one or more His tags.

### Exemplary ABEs

In some aspects, the base editing methods of the disclosure comprise the use of an adenine base editor. Exemplary adenine base editors include, but are not limited to, ABE7.10 (or ABEmax), ABE8e, ABE8e-SaKKH, ABE8e-NG, ABE-xCas9, ABE7.10-SaKKH, ABE7.10-NG, ABE7.10-VRQR, ABE7.10-VQR, ABE8e-NRTH, ABE8e-NRRH, ABE8e-VQR, or ABE8e-VRQR. In certain embodiments, the adenine base editor used in the disclosed methods is an ABE8e or an ABE7.10. ABE8e may be referred to herein as “ABE8” or “ABE8.0”. The ABE8e base editor and variants thereof may comprise an adenosine deaminase domain containing a TadA-8e adenosine deaminase monomer (monomer form) or a TadA-8e adenosine deaminase homodimer or heterodimer (dimer form). Other ABEs may be used to deaminate an A nucleobase in accordance with the disclosed methods.

In some aspects, the disclosure provides complexes of adenine base editors and guide RNAs. Exemplary adenine base editors of the disclosed complexes include, but are not limited to, ABE7.10 (or ABEmax), ABE8e, ABE8e-SaKKH, ABE8e-NG, ABE-xCas9, ABE7.10-SaKKH, ABE7.10-NG, ABE7.10-VRQR, ABE7.10-VQR, ABE8e-NRTH, ABE8e-NRRH, ABE8e-VQR, or ABE8e-VRQR. In certain embodiments, the adenine base editor of any of the disclosed complexes is a ABE8e or an ABE7.10. Other ABEs may be used to deaminate a A nucleobase in accordance with the disclosed complexes.

The disclosed complexes of ABEs may possess an on-target editing efficiency of more than 50% after being contacted with a nucleic acid molecule comprising a target sequence. Further exemplary ABE complexes possess an on-target editing efficiency of more than 60% after being contacted with a nucleic acid molecule comprising a target sequence. Further exemplary ABEs possess an on-target editing efficiency of more than 65%, more than 70%, more than 75%, more than 80%, more than 82.5%, or more than 85% after being contacted with a nucleic acid molecule comprising a target sequence. The disclosed ABE complexes may exhibit indel frequencies of less than 0.75%, less than 0.6%, less than 0.5%, less than 0.4%, less than 0.3%, or less than 0.2% after being contacted with a nucleic acid molecule containing a target sequence.

Some aspects of the disclosure provide base editors that comprise a nucleic acid programmable DNA binding protein (napDNAbp) and at least two adenosine deaminase domains. Without wishing to be bound by any particular theory, dimerization of adenosine deaminases (e.g., in cis or in trans) may improve the ability (e.g., efficiency) of the base editor to modify a nucleic acid base, for example to deaminate adenine. In some embodiments, any of the base editors may comprise 2, 3, 4 or 5 adenosine deaminase domains. In some embodiments, any of the base editors described herein comprises two adenosine deaminases. In some embodiments, any of the base editors described herein contains only two adenosine deaminases. In some embodiments, the adenosine deaminases are the same. In some embodiments, the adenosine deaminases are any of the adenosine deaminases described herein. In some embodiments, the adenosine deaminases are different.

In some embodiments, the first adenosine deaminase is any of the adenosine deaminases described herein, and the second adenosine is any of the adenosine deaminases described herein, but is not identical to the first adenosine deaminase. As one example, the base editor may comprise a first adenosine deaminase and a second adenosine deaminase that both comprise the amino acid sequence of SEQ ID NO: 10, which contains a W23R; H36L; P48A; R51L; L84F; A106V; D108N; H123Y; S146C; D147Y; R152P; E155V; I156F; and K157N mutation from ecTadA (SEQ ID NO: 1). In some embodiments, the base editor may comprise a first adenosine deaminase that comprises the amino acid sequence of SEQ ID NO: 1, and a second adenosine deaminase domain that comprises the amino acid sequence of TadA7.10 of SEQ ID NO: 10. In certain embodiments, the first and/or second deaminase is a TadA-8e deaminase. Additional base editor constructs comprising two adenosine deaminase domains are illustrated herein and are provided in the art.

In some embodiments, the base editor comprises two adenosine deaminases (e.g., a first adenosine deaminase and a second adenosine deaminase). In some embodiments, the base editor comprises a first adenosine deaminase and a second adenosine deaminase. In some embodiments, the first adenosine deaminase is N-terminal to the second adenosine deaminase in the base editor. In some embodiments, the first adenosine deaminase is C-terminal to the second adenosine deaminase in the base editor. In some embodiments, the first adenosine deaminase and the second deaminase are fused directly or via a linker. In some embodiments, the linker is any of the linkers provided herein, for example, any of the linkers described in the “Linkers” section. In some embodiments, the linker comprises the amino acid sequence of any one of SEQ ID NOs: 135-152. In some embodiments, the linker is 32 amino acids in length. In some embodiments, the linker comprises the amino acid sequence (SGGS)₂-SGSETPGTSESATPES-(SGGS)₂ (SEQ ID NO: 136), which may also be referred to as (SGGS)₂-XTEN-(SGGS)₂ (SEQ ID NO: 136). In some embodiments, the linker comprises the amino acid sequence (SGGS).-SGSETPGTSESATPES-(SGGS)ₙ (SEQ ID NO: 142), wherein n is 0, 1, 2, 3, 4, 5, 6, 7, 8, 9, or 10. In some embodiments, the first adenosine deaminase is the same as the second adenosine deaminase. In some embodiments, the first adenosine deaminase and the second adenosine deaminase are any of the adenosine deaminases described herein. In some embodiments, the first adenosine deaminase and the second adenosine deaminase are different. In some embodiments, the first adenosine deaminase is any of the adenosine deaminases described herein. In some embodiments, the second adenosine deaminase is any of the adenosine deaminases described herein but is not identical to the first adenosine deaminase. In some embodiments, the first adenosine deaminase is an ecTadA adenosine deaminase. In some embodiments, the first adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 1-10, or to any of the adenosine deaminases described herein. In some embodiments, the first adenosine deaminase comprises the amino acid sequence of SEQ ID NO: 1. In some embodiments, the second adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 1-10, or to any of the adenosine deaminases described herein. In some embodiments, the second adenosine deaminase comprises the amino acid sequence of SEQ ID NO: 10.

In some embodiments, exemplary ABEs for use in the present disclosure include those from Gaudelli et al. Nature Biotechnology (2020) 38, 892-900, and International Patent Pub. No. WO 2020/231863, published Nov. 19, 2020, each of which is incorporated by reference herein. These base editors include, but are not limited to, a base editor comprising a sequence that is at least 90%, at least 95%, at least 98%, at least 99%, or at least 99.5% identical to the sequences reported therein for base editors ABE8.8-m, ABE8.13-m, ABE8.17-m, ABE8.20-m, ABE8.8d, ABE8.13-m, ABE8.17-d, and ABE8.20-d.

In some embodiments, exemplary ABEs for use in the present disclosure include those provided in WO 2020/051562, published Mar. 12, 2020, which discloses multi-effector cytosine base editors having two napDNAbp domains (dCas9 and Cas9n), adenosine deaminase domain and and a UGI domain. In some embodiments, exemplary ABEs for use in the present disclosure include those provided in WO 2020/028823, published Feb. 6, 2020, which discloses multi-effector base editors having a pmCDA cytidine deaminase domain, a TadA7.10 adenosine deaminase domain, a UGI domain, and a Cas9n domain. Each of these publications is incorporated herein by reference.

In some embodiments, the general architecture of exemplary base editors with a first adenosine deaminase, a second adenosine deaminase, and a napDNAbp comprises any one of the following structures, where NLS is a nuclear localization sequence (e.g., any NLS provided herein), NH₂ is the N-terminus of the base editor, and COOH is the C-terminus of the base editor.

Base editors comprising a first adenosine deaminase, a second adenosine deaminase, and a napDNAbp. NH₂-[first adenosine deaminase]-[second adenosine deaminase]-[napDNAbp]-COOH; NH₂-[first adenosine deaminase]-[napDNAbp]-[second adenosine deaminase]-COOH; NH₂-[napDNAbp]-[first adenosine deaminase]-[second adenosine deaminase]-COOH; NH₂-[second adenosine deaminase]-[first adenosine deaminase]-[napDNAbp]-COOH; NH₂-[second adenosine deaminase]-[napDNAbp]-[first adenosine deaminase]-COOH; NH₂-[napDNAbp]-[second adenosine deaminase]-[first adenosine deaminase]-COOH.

In some embodiments, the base editors described herein do not comprise a linker. In some embodiments, a linker is present between one or more of the domains or proteins (e.g., first adenosine deaminase, second adenosine deaminase, and/or napDNAbp). In some embodiments, the “]-[” used in the general architecture above indicates the presence of an optional linker.

Base editors comprising a first adenosine deaminase, a second adenosine deaminase, a napDNAbp, and an NLS. NH₂-[NLS]-[first adenosine deaminase]-[second adenosine deaminase]-[napDNAbp]-COOH; NH₂-[first adenosine deaminase]-[NLS]-[second adenosine deaminase]-[napDNAbp]-COOH; NH₂-[first adenosine deaminase]-[second adenosine deaminase]-[NLS]-[napDNAbp]-COOH; NH₂-[first adenosine deaminase]-[second adenosine deaminase]-[napDNAbp]-[NLS]-COOH; NH₂-[NLS]-[first adenosine deaminase]-[napDNAbp]-[second adenosine deaminase]-COOH; NH₂-[first adenosine deaminase]-[NLS]-[napDNAbp]-[second adenosine deaminase]-COOH; NH₂-[first adenosine deaminase]-[napDNAbp]-[NLS]-[second adenosine deaminase]-COOH; NH₂-[first adenosine deaminase]-[napDNAbp]-[second adenosine deaminase]-[NLS]-COOH; NH₂-[NLS]-[napDNAbp]-[first adenosine deaminase]-[second adenosine deaminase]-COOH; NH₂-[napDNAbp]-[NLS ]-[first adenosine deaminase]-[second adenosine deaminase]-COOH; NH₂-[napDNAbp]-[first adenosine deaminase]-[NLS]-[second adenosine deaminase]-COOH; NH₂-[napDNAbp]-[first adenosine deaminase]-[second adenosine deaminase]-[NLS ]-COOH; NH₂-[NLS]-[second adenosine deaminase]-[first adenosine deaminase]-[napDNAbp]-COOH; NH₂-[second adenosine deaminase]-[NLS]-[first adenosine deaminase]-[napDNAbp]-COOH; NH₂-[second adenosine deaminase]-[first adenosine deaminase]-[NLS]-[napDNAbp]-COOH; NH₂-[second adenosine deaminase]-[first adenosine deaminase]-[napDNAbp]-[NLS]-COOH; NH₂-[NLS]-[second adenosine deaminase]-[napDNAbp]-[first adenosine deaminase]-COOH; NH₂-[second adenosine deaminase]-[NLS ]-[napDNAbp]-[first adenosine deaminase]-COOH; NH₂-[second adenosine deaminase]-[napDNAbp]-[NLS ]-[first adenosine deaminase]-COOH; NH₂-[second adenosine deaminase]-[napDNAbp]-[first adenosine deaminase]-[NLS]-COOH; NH₂-[NLS]-[napDNAbp]-[second adenosine deaminase]-[first adenosine deaminase]-COOH; NH₂-[napDNAbp]-[NLS ]-[second adenosine deaminase]-[first adenosine deaminase]-COOH; NH₂-[napDNAbp]-[second adenosine deaminase]-[NLS]-[first adenosine deaminase]-COOH; NH₂-[napDNAbp]-[second adenosine deaminase]-[first adenosine deaminase]-[NLS]-COOH.

Exemplary ABEs include, without limitation, the following base editors. For the purposes of clarity, the adenosine deaminase domain may be shown in Bold; mutations of the ecTadA deaminase domain are shown in Bold underlining; the XTEN linker is shown in italics; the UGI/AAG/EndoV domains are shown in Bold italics; and NLS is shown in underlined italics:

ecTadA(wt)-XTEN-nCas9-NLS:
(SEQ ID NO: 373)
MSEVEFSHEYWMRHALTLAKRAWDEREVPVGAVLVHNNRVIGEGWNRPIGRHDPTAHAEIMAL
RQGGLVMQNYRLIDATLYVTLEPCVMCAGAMIHSRIGRVVFGARDAKTGAAGSLMDVLHHPGM
NHRVEITEGILADECAALLSDFFRMRRQEIKAQKKAQSSTDSGSETPGTSESATPESDKKYSI
GLAIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKH
SLLYEYFTVYNELTKVKYVTEGMRKPAFLSGEQKKAIVDLLFKTNRKVTVKQLKEDYFKKIEC
FDSVEISGVEDRFNASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETGEIVWDKGR
DFATVRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQLFVEQHKHYLD
EIIEQISEFSKRVILADANLDKVLSAYNKHRDKPIREQAENIIHLFTLTNLGAPAAFKYFDTT
IDRKRYTSTKEVLDATLIHQSITGLYETRIDLSQLGGDSGGSPKKKRKV
ecTadA(D108N)-XTEN-nCas9-NLS: (mammalian construct,
active on DNA, A to G editing):
(SEQ ID NO: 374)
MSEVEFSHEYWMRHALTLAKRAWDEREVPVGAVLVHNNRVIGEGWNRPIGRHDPTAHAEIMAL
RQGGLVMQNYRLIDATLYVTLEPCVMCAGAMIHSRIGRVVFGARNAKTGAAGSLMDVLHHPGM
NHRVEITEGILADECAALLSDFFRMRRQEIKAQKKAQSSTDSGSETPGTSESATPESDKKYSI
GLAIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKH
SLLYEYFTVYNELTKVKYVTEGMRKPAFLSGEQKKAIVDLLFKTNRKVTVKQLKEDYFKKIEC
FDSVEISGVEDRFNASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETGEIVWDKGR
DFATVRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQLFVEQHKHYLD
EIIEQISEFSKRVILADANLDKVLSAYNKHRDKPIREQAENIIHLFTLTNLGAPAAFKYFDTT
IDRKRYTSTKEVLDATLIHQSITGLYETRIDLSQLGGDSGGSPKKKRKV
ecTadA(D108G)-XTEN-nCas9-NLS: (mammalian construct,
active on DNA, A to G editing):
(SEQ ID NO: 375)
MSEVEFSHEYWMRHALTLAKRAWDEREVPVGAVLVHNNRVIGEGWNRPIGRHDPTAHAEIMAL
RQGGLVMQNYRLIDATLYVTLEPCVMCAGAMIHSRIGRVVFGARGAKTGAAGSLMDVLHHPGM
NHRVEITEGILADECAALLSDFFRMRRQEIKAQKKAQSSTDSGSETPGTSESATPESDKKYSI
GLAIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKH
SLLYEYFTVYNELTKVKYVTEGMRKPAFLSGEQKKAIVDLLFKTNRKVTVKQLKEDYFKKIEC
FDSVEISGVEDRFNASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETGEIVWDKGR
DFATVRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQLFVEQHKHYLD
EIIEQISEFSKRVILADANLDKVLSAYNKHRDKPIREQAENIIHLFTLTNLGAPAAFKYFDTT
IDRKRYTSTKEVLDATLIHQSITGLYETRIDLSQLGGDSGGSPKKKRKV
ecTadA(D108V)-XTEN-nCas9-NLS: (mammalian construct,
active on DNA, A to G editing):
(SEQ ID NO: 376)
MSEVEFSHEYWMRHALTLAKRAWDEREVPVGAVLVHNNRVIGEGWNRPIGRHDPTAHAEIMAL
RQGGLVMQNYRLIDATLYVTLEPCVMCAGAMIHSRIGRVVFGARVAKTGAAGSLMDVLHHPGM
NHRVEITEGILADECAALLSDFFRMRRQEIKAQKKAQSSTDSGSETPGTSESATPESDKKYSI
GLAIGTNSVGWAVITDEYKVPSKKFKVLGNTDRHSIKKNLIGALLFDSGETAEATRLKRTARR
RYTRRKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLSASMI
KRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKH
SLLYEYFTVYNELTKVKYVTEGMRKPAFLSGEQKKAIVDLLFKTNRKVTVKQLKEDYFKKIEC
FDSVEISGVEDRFNASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETGEIVWDKGR
DFATVRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQLFVEQHKHYLD
EIIEQISEFSKRVILADANLDKVLSAYNKHRDKPIREQAENIIHLFTLTNLGAPAAFKYFDTT
IDRKRYTSTKEVLDATLIHQSITGLYETRIDLSQLGGDSGGSPKKKRKV
ABE7.10: ecTadA_{(wild-type)}-(SGGS)₂-XTEN-(SGGS)₂-
ecTadA_{(W23R}_H36L_P48A_R51L_L84F_A106V_D108N_H123Y_
_{S146C}_D147Y_R152P_E155V_I156F_K157N)-(SGGS)2-XTEN-
(SGGS)₂_nCas9_SGGS_NLS
(SEQ ID NO: 377)
MSEVEFSHEYWMRHALTLAKRAWDEREVPVGAVLVHNNRVIGEGWNRPIGRHDPTAHAEIMAL
RQGGLVMQNYRLIDATLYVTLEPCVMCAGAMIHSRIGRVVFGARDAKTGAAGSLMDVLHHPGM
NHRVEITEGILADECAALLSDFFRMRRQEIKAQKKAQSSTDSGGSSGGSSGSETPGTSESATP
ESSGGSSGGSSEVEFSHEYWMRHALTLAKRARDEREVPVGAVLVLNNRVIGEGWNRAIGLHDP
TAHAEIMALRQGGLVMQNYRLIDATLYVTFEPCVMCAGAMIHSRIGRVVFGVRNAKTGAAGSL
MDVLHYPGMNHRVEITEGILADECAALLCYFFRMPRQVFNAQKKAQSSTDSGGSSGGSSGSET
PGTSESATPESSGGSSGGSDKKYSIGLAIGTNSVGWAVITDEYKVPSKKFKVLGNTDRHSIKK
NLIGALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHFLIE
GDLNPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAAKNLS
DAILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPAIKKGIL
QTVKVVDELVKVMGRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDSIDNKVLTRSDKNR
GKSDNVPSEEVVKKMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNPI
DFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQ
AENIIHLFTLTNLGAPAAFKYFDTTIDRKRYTSTKEVLDATLIHQSITGLYETRIDLSQLGGD
SGGSPKKKRKV
ABEmax (7.10): NLS_ecTadA_{(wild-type)}-(SGGS)₂-XTEN-
(SGGS)₂-ecTadA7.10_{(W23R}_H36L_P48A_R51L_L84F_A106V_
_{D108N}_H123Y_S146C_D147Y_R152P_E155V_I156F_K157N)-
(SGGS)₂-XTEN-(SGGS)₂_nCas9 VRQR_SGGS_NLS
(SEQ ID NO: 378)
MKRTADGSEFESPKKKRKVSEVEFSHEYWMRHALTLAKRAWDEREVPVGAVLVHNNRVIGEGW
NRPIGRHDPTAHAEIMALRQGGLVMQNYRLIDATLYVTLEPCVMCAGAMIHSRIGRVVFGARD
AKTGAAGSLMDVLHHPGMNHRVEITEGILADECAALLSDFFRMRRQEIKAQKKAQSSTDSGGS
SGGSSGSETPGTSESATPESSGGSSGGSSEVEFSHEYWMRHALTLAKRARDEREVPVGAVLVL
NNRVIGEGWNRAIGLHDPTAHAEIMALRQGGLVMQNYRLIDATLYVTFEPCVMCAGAMIHSRI
GRVVFGVRNAKTGAAGSLMDVLHYPGMNHRVEITEGILADECAALLCYFFRMPRQVFNAQKKA
QSSTDSGGSSGGSSGSETPGTSESATPESSGGSSGGSDKKYSIGLAIGTNSVGWAVITDEYKV
PSKKFKVLGNTDRHSIKKNLIGALLFDSGETAEATRLKRTARRRYTRRKNRICYLQEIFSNEM
AKVDDSFFHRLEESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQLSKDTYDDDLDNLL
AQIGDQYADLFLAAKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRERMKR
IEEGIKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKT
EVQTGGFSKESILPKRNSDKLIARKKDWDPKKYGGFVSPTVAYSVLVVAKVEKGKSKKLKSVK
ELLGITIMERSSFEKNPIDFLEAKGYKEVKKDLIIKLPKYSLFELENGRKRMLASARELQKGN
ELALPSKYVNFLYLASHYEKLKGSPEDNEQKQLFVEQHKHYLDEIIEQISEFSKRVILADANL
DKVLSAYNKHRDKPIREQAENIIHLFTLTNLGAPAAFKYFDTTIDRKQYRSTKEVLDATLIHQ
SITGLYETRIDLSQLGGDSGGSKRTADGSEFEPKKKRKV
ABE8e (monomer)
(SEQ ID NO: 379)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRN
SKRGAAGSLMNVLNYPGMNHRVEITEGILADECAALLCDFYRMPRQVFNAQKKAQSSINSGGS
SGGSSGSETPGTSESATPESSGGSSGGSDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIANLAG
SPAIKKGILQTVKVVDELVKVMGRHKPENIVIEMARENQTTQKGQKNSRERMKRIEEGIKELG
SQILKEHPVENTQLQNEKLYLYYLQNGRDMYVDQELDINRLSDYDVDHIVPQSFLKDDSIDNK
VLTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKLKSVKELLGITIME
RSSFEKNPIDFLEAKGYKEVKKDLIIKLPKYSLFELENGRKRMLASAGELQKGNELALPSKYV
NFLYLASHYEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQSITGLYETR
IDLSQLGGDSGGSKRTADGSEFEPKKKRKV
ABE8e (dimer)
(SEQ ID NO: 380)
MKRTADGSEFESPKKKRKVSEVEFSHEYWMRHALTLAKRAWDEREVPVGAVLVHNNRVIGEGW
NRPIGRHDPTAHAEIMALRQGGLVMQNYRLIDATLYVTLEPCVMCAGAMIHSRIGRVVFGARD
AKTGAAGSLMDVLHHPGMNHRVEITEGILADECAALLSDFFRMRRQEIKAQKKAQSSTDSGGS
SGGSSGSETPGTSESATPESSGGSSGGSSEVEFSHEYWMRHALTLAKRARDEREVPVGAVLVL
NNRVIGEGWNRAIGLHDPTAHAEIMALRQGGLVMQNYRLIDATLYVTFEPCVMCAGAMIHSRI
GRVVFGVRNSKRGAAGSLMNVLNYPGMNHRVEITEGILADECAALLCDFYRMPRQVFNAQKKA
QSSINSGGSSGGSSGSETPGTSESATPESSGGSSGGSDKKYSIGLAIGTNSVGWAVITDEYKV
PSKKFKVLGNTDRHSIKKNLIGALLFDSGETAEATRLKRTARRRYTRRKNRICYLQEIFSNEM
AKVDDSFFHRLEESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQLSKDTYDDDLDNLL
AQIGDQYADLFLAAKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRERMKR
IEEGIKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANL
DKVLSAYNKHRDKPIREQAENIIHLFTLTNLGAPAAFKYFDTTIDRKRYTSTKEVLDATLIHQ
SITGLYETRIDLSQLGGDSGGSKRTADGSEFEPKKKRKV
SaABE8e
(SEQ ID NO: 381)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRN
SKRGAAGSLMNVLNYPGMNHRVEITEGILADECAALLCDFYRMPRQVFNAQKKAQSSINSGGS
SGGSSGSETPGTSESATPESSGGSSGGSGKRNYILGLAIGITSVGYGIIDYETRDVIDAGVRL
FKEANVENNEGRRSKRGARRLKRRRRHRIQRVKKLLFDYNLLTDHSELSGINPYEARVKGLSQ
KLSEEEFSAALLHLAKRRGVHNVNEVEEDTGNELSTKEQISRNSKALEEKYVAELQLERLKKD
GEVRGSINRFKTSDYVKEAKQLLKVQKAYHQLDQSFIDTYIDLLETRRTYYEGPGEGSPFGWK
DIKEWYEMLMGHCTYFPEELRSVKYAYNADLYNALNDLNNLVITRDENEKLEYYEKFQIIENV
FKQKKKPTLKQIAKEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAINLILDELWHTNDN
QIAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLE
AIPLEDLLNNPFNYEVDHIIPRSVSFDNSFNNKVLVKQEENSKKGNRTPFQYLSSSDSKISYE
TFKKHILNLAKGKGRISKTKKEYLLEERDINRFSVQKDFINRNLVDTRYATRGLMNLLRSYFR
VNNLDVKVKSINGGFTSFLRRKWKFKKERNKGYKHHAEDALIIANADFIFKEWKKLDKAKKVM
ENQMFEEKQAESMPEIETEQEYKEIFITPHQIKHIKDFKDYKYSHRVDKKPNRELINDTLYST
RKDDKGNTLIVNNLNGLYDKDNDKLKKLINKSPEKLLMYHHDPQTYQKLKLIMEQYGDEKNPL
YKYYEETGNYLTKYSKKDNGPVIKKIKYYGNKLNAHLDITDDYPNSRNKVVKLSLKPYRFDVY
LDNGVYKFVTVKNLDVIKKENYYEVNSKCYEEAKKLKKISNQAEFIASFYNNDLIKINGELYR
VIGVNNDLLNRIEVNMIDITYREYLENMNDKRPPRIIKTIASKTQSIKKYSTDILGNLYEVKS
KKHPQIIKKGSGGSKRTADGSEFEPKKKRKV
SpCas9NG-ABE8e (“ABE8e-NG”)
(SEQ ID NO: 382)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRN
SKRGAAGSLMNVLNYPGMNHRVEITEGILADECAALLCDFYRMPRQVFNAQKKAQSSINSGGS
SGGSSGSETPGTSESATPESSGGSSGGSDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIANLAG
SPAIKKGILQTVKVVDELVKVMGRHKPENIVIEMARENQTTQKGQKNSRERMKRIEEGIKELG
SQILKEHPVENTQLQNEKLYLYYLQNGRDMYVDQELDINRLSDYDVDHIVPQSFLKDDSIDNK
VLTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNIVKKTEVQTGGFSK
ESIRPKRNSDKLIARKKDWDPKKYGGFVSPTVAYSVLVVAKVEKGKSKKLKSVKELLGITIME
RSSFEKNPIDFLEAKGYKEVKKDLIIKLPKYSLFELENGRKRMLASARFLQKGNELALPSKYV
NFLYLASHYEKLKGSPEDNEQKQLFVEQHKHYLDEIIEQISEFSKRVILADANLDKVLSAYNK
HRDKPIREQAENIIHLFTLTNLGAPRAFKYFDTTIDRKVYRSTKEVLDATLIHQSITGLYETR
IDLSQLGGDSGGSKRTADGSEFEPKKKRKV
SaKKH-ABE8e (“ABE8e-KKH”)
(SEQ ID NO: 383)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRN
SKRGAAGSLMNVLNYPGMNHRVEITEGILADECAALLCDFYRMPRQVFNAQKKAQSSINSGGS
SGGSSGSETPGTSESATPESSGGSSGGSGKRNYILGLAIGITSVGYGIIDYETRDVIDAGVRL
FKEANVENNEGRRSKRGARRLKRRRRHRIQRVKKLLFDYNLLTDHSELSGINPYEARVKGLSQ
KLSEEEFSAALLHLAKRRGVHNVNEVEEDTGNELSTKEQISRNSKALEEKYVAELQLERLKKD
GEVRGSINRFKTSDYVKEAKQLLKVQKAYHQLDQSFIDTYIDLLETRRTYYEGPGEGSPFGWK
DIKEWYEMLMGHCTYFPEELRSVKYAYNADLYNALNDLNNLVITRDENEKLEYYEKFQIIENV
FKQKKKPTLKQIAKEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAINLILDELWHTNDN
QIAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLE
AIPLEDLLNNPFNYEVDHIIPRSVSFDNSFNNKVLVKQEENSKKGNRTPFQYLSSSDSKISYE
TFKKHILNLAKGKGRISKTKKEYLLEERDINRFSVQKDFINRNLVDTRYATRGLMNLLRSYFR
VNNLDVKVKSINGGFTSFLRRKWKFKKERNKGYKHHAEDALIIANADFIFKEWKKLDKAKKVM
ENQMFEEKQAESMPEIETEQEYKEIFITPHQIKHIKDFKDYKYSHRVDKKPNRKLINDTLYST
RKDDKGNTLIVNNLNGLYDKDNDKLKKLINKSPEKLLMYHHDPQTYQKLKLIMEQYGDEKNPL
YKYYEETGNYLTKYSKKDNGPVIKKIKYYGNKLNAHLDITDDYPNSRNKVVKLSLKPYRFDVY
LDNGVYKFVTVKNLDVIKKENYYEVNSKCYEEAKKLKKISNQAEFIASFYKNDLIKINGELYR
VIGVNNDLLNRIEVNMIDITYREYLENMNDKRPPHIIKTIASKTQSIKKYSTDILGNLYEVKS
KKHPQIIKKGSGGSKRTADGSEFEPKKKRKV
ABE8-NRTTH: NLS, TadA, linker, TadA, NRTH
(SEQ ID NO: 384)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRN
SKRGAAGSLMNVLNYPGMNHRVEITEGILADECAALLCDFYRMPRQVFNAQKKAQSSINSGGS
SGGSSGSETPGTSESATPESSGGSSGGSSEVEFSHEYWMRHALTLAKRARDEREVPVGAVLVL
NNRVIGEGWNRAIGLHDPTAHAEIMAERQGGEVMQNYREIDATEYVTFEPCVMCAGAMIHSRI
GRWFGVRNSKRGAAGSEMNVENYPGMNHRVEITEGIEADECAAEECDFYRMPRQVFNAQKKAQ
SSINSGGSSGGSSGSETPGTSESATPESSGGSSGGSDKKYSIGETIGTNSVGWAVITDEYKVP
SKKFKVEGNTDRHSIKKNEIGAEEFDSGETAEATREKRTARRRYTRRKNRICYEQEIFSNEMA
KVDDSFFHREEESFEVEEDKKHERHPIFGNIVDEVAYHEKYPTIYHERKKEVDSTDKADEREI
YEAEAHMIKFRGHFEIEGDENPDNSDVDKEFIQEVQTYNQEFEENPINASGVDAKAIESARES
KSRREENEIAQEPGEKKNGEFGNEIAESEGETPNFKSNFDEAEDAKEQESKDTYDDDEDNEEA
QIGDQYADEFEAAKNESDAIEESDIERVNTEITKAPESASMVKRYDEHHQDETEEKAEVRQQE
PEKYKEIFFDQSKNGYAGYIDGGASQEEFYKFIKPIEEKMDGTEEEEVKENREDEERKQRTFD
NGIIPHQIHLGELHAILRRQGDFYPFLKDNREKIEKILTFRIPYYVGPLARGNSRFAWMTRKS
EETITPWNFEEVVDKGASAQSFIERMTNFDKNEPNEKVEPKHSEEYEYFTVYNEETKVKYVFE
GMRKPAFESGEQKKAIVDEEFKTNRKVTVKQEKEDYFKKIECFDSVEISGVEDRFNASEGTYH
DEEKIIKDKDFEDNEENEDIEEDIVETETEFEDREMIEEREKTYAHEFDDKVMKQEKRERYTG
WGRESRKEINGIRDKQSGKTIEDFEKSDGFANRNFMQEIHDDSETFKEDIQKAQVSCQGDSEH
EHIANEAGSPAIKKGIEQTVKWDEEVKVMGGHKPENIVIEMARENQTTQKGQKNSRERMKRIE
EGIKEEGSQIEKEHPVENTQEQNEKEYEYYEQNGRDMYVDQEEDINRESDYDVDHIVPQSFEK
DDSIDNKVETRSDKNRGKSDNVPSEEVVKKMKNYWRQEENAKEITQRKFDNETKAERGGESEE
DKAGFIKRQEVETRQITKHVAQIEDSRMNTKYDENDKEIREVKVITEKSKEVSDFRKDFQFYK
VREINNYHHAHDAYENAVVGTAEIKKYPKEESEFVYGDYKVYDVRKMIAKSEQEIGKATAKYF
FYSNIMNFFKTEITEANGEIRKRPEIETNGETGEIVWDKGRDFATVRKVESMPQVNIVKKTEV
QTGGFSKESIEPKGNSDKEIARKKDWDPKKYGGFNSPTVAYSVEVVAKVEKGKSKKEKSVKEE
EGITIMERSSFEKNPIGFEEAKGYKEVKKDEIIKEPKYSEFEEENGRKRMEASASVEHKGNEE
AEPSKYVNFEYEASHYEKEKGSSEDNKQKQEFVEQHKHYEDEIIEQISEFSKRVIEADANEDK
VESAYNKHRDKPIREQAENIIHEFTETNEGASAAFKYFDTTIGRKLYTSTKEVLDATLIHQSI
TGLYETRIDLSQLGGDSGGSKRTADGSEFEPKKKRKV
ABE8-NRRH: NLS, TadA, linker, TadA, NRRH
(SEQ ID NO: 385)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRN
SKRGAAGSLMNVLNYPGMNHRVEITEGILADECAALLCDFYRMPRQVFNAQKKAQSSINSGGS
SGGSSGSETPGTSESATPESSGGSSGGSSEVEFSHEYWMRHAETEAKRARDEREVPVGAVEVE
NNRVIGEGWNRAIGEHDPTAHAEIMAERQGGEVMQNYREIDATEYVTFEPCVMCAGAMIHSRI
GRVVFGVRNSKRGAAGSEMNVENYPGMNHRVEITEGIEADECAAEECDFYRMPRQVFNAQKKA
QSSINSGGSSGGSSGSETPGESESAEPESSGGSSGGSDKKYSIGLTIGTNSVGWAVITDEYKV
PSKKFKVLGNTDRHSIKKNLIGALLFDSGETAEATRLKRTARRRYTRRKNRICYLQEIFSNEM
AKVDDSFFHRLEESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQLSKDTYDDDLDNLL
AQIGDQYADLFLAAKNLSDAILLSDILRVNTEITKAPLSASMVKRYDEHHQDLTLLKALVRQQ
LPEKYKEIFFDQSKNGYAGYIDGGASQEEFYKFIKPILEKMDGTEELLVKLNREDLLRKQRTF
DNGIIPHQIHLGELHAILRRQGDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELTKVKYVF
EGMRKPAFESGEQKKAIVDEEFKTNRKVTVKQEKEDYFKKIECFDSVEISGVEDRFNASEGTY
HDEEKIIKDKDFEDNEENEDIEEDIVETETEFEDREMIEEREKTYAHEFDDKVMKQEKRERYT
GWGRESRKEINGIRDKQSGKTIEDFEKSDGFANRNFMQEIHDDSETFKEDIQKAQVSCQGDSE
HEHIANEAGSPAIKKGIEQTVKWDEEVKVMGGHKPENIVIEMARENQTTQKGQKNSRERMKRI
EEGIKEEGSQIEKEHPVENTQEQNEKEYEYYEQNGRDMYVDQEEDINRESDYDVDHIVPQSFE
KDDSIDNKVETRSDKNRGKSDNVPSEEVVKKMKNYWRQEENAKEITQRKFDNETKAERGGESE
EDKAGFIKRQEVETRQITKHVAQIEDSRMNTKYDENDKEIREVKVITEKSKEVSDFRKDFQFY
KVREINNYHHAHDAYENAVVGTAEIKKYPKEESEFVYGDYKVYDVRKMIAKSEQEIGKATAKY
FFYSNIMNFFKTEITEANGEIRKRPEIETNGETGEIVWDKGRDFATVRKVESMPQVNIVKKTE
VQTGGFSKESIEPKGNSDKEIARKKDWDPKKYGGFNSPTAAYSVEVVAKVEKGKSKKEKSVKE
EEGITIMERSSFEKNPIGFEEAKGYKEVKKDEIIKEPKYSEFEEENGRKRMEASAGVEHKGNE
EAEPSKYVNFEYEASHYEKEKGSPEDNEQKQEFVEQHKHYEDEIIEQISEFSKRVIEADANED
KVESAYNKHRDKPIREQAENIIHEFTETNEGVPAAFKYFDTTIDKKRYTSTKEVLDATLIHQS
ITGLYETRIDLSQLGGDSGGSKRTADGSEFEPKKKRKV
xCas9(3.7)-ABE(7.10): ( [symbol] - [symbol] -
ecTadA*(7.10)- [symbol] -nxCas9(3.7)-NLS):
(SEQ ID NO: 386)

[symbol]

[symbol]

[symbol]

[symbol]

[symbol]

[symbol]

[symbol]

[symbol]

[symbol]

[symbol]

[symbol]

SEVEFSHEYWMRHALTLAKRARDEREVPVGA
VEVENNRVIGEGWNRAIGEHDPTAHAEIMAERQGGEVMQNYREIDATEYVTFEPCVMCAGAMI
HSRIGRWFGVRNAKTGAAGSEMDVEHYPGMNHRVEITEGIEADECAAEECYFFRMPRQVFNAQ
KKAQSSTD [symbol] [symbol] DKKYSIGLAIGTNSVGWAVITDE
YKVPSKKFKVLGNTDRHSIKKNLIGALLFDSGETAEATRLKRTARRRYTRRKNRICYLQEIFS
NEMAKVDDSFFHRLEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENPINASGVDAKAILS
ARLSKSRRLENLIAQLPGEKKNGLFGNLIALSLGLTPNFKSNFDLAEDTKLQLSKDTYDDDLD
NLLAQIGDQYADLFLAAKNLSDAILLSDILRVNTEITKAPLSASMIKLYDEHHQDLTLLKALV
RQQLPEKYKEIFFDQSKNGYAGYIDGGASQEEFYKFIKPILEKMDGTEELLVKLNREDLLRKQ
RTFDNGIIPHQIHLGELHAILRRQEDFYPFLKDNREKIEKILTFRIPYYVGPLARGNSRFAWM
TRKSEETITPWNFEKVVDKGASAQSFIERMTNFDKNLPNEKVLPKHSLLYEYFTVYNELTKVK
YVTEGMRKPAFLSGDQKKAIVDLLFKTNRKVTVKQLKEDYFKKIECFDSVEISGVEDRFNASL
GTYHDLLKIIKDKDFLDNEENEDILEDIVLTLTLFEDREMIEERLKTYAHLFDDKVMKQLKRR
RYTGWGRLSRKLINGIRDKQSGKTILDFLKSDGFANRNFIQLIHDDSLTFKEDIQKAQVSGQG
DSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVITLKSKLVSDFRKD
FQFYKVREINNYHHAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFATVRKVLSMPQVNIV
KKTEVQTGGFSKESILPKRNSDKLIARKKDWDPKKYGGFDSPTVAYSVLVVAKVEKGKSKKLK
SVKELLGITIMERSSFEKNPIDFLEAKGYKEVKKDLIIKLPKYSLFELENGRKRMLASAGVLQ
KGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATL
IHQSITGLYETRIDLSQLGGD [symbol] PKKKRKV
ABE8-VRQR: NFS, TadA, linker, TadA, SpCas9-VRQR
(SEQ ID NO: 387)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRN
SKRGAAGSLMNVLNYPGMNHRVEITEGILADECAALLCDFYRMPRQVFNAQKKAQSSINSGGS
SGGSSGSETPGTSESATPESSGGSSGGSSEVEFSHEYWMRHALTLAKRARDEREVPVGAVLVL
NNRVIGEGWNRAIGLHDPTAHAEIMAERQGGEVMQNYREIDATEYVTFEPCVMCAGAMIHSRI
GRWFGVRNSKRGAAGSEMNVENYPGMNHRVEITEGIEADECAAEECDFYRMPRQVFNAQKKAQ
SSINSGGSSGGSSGSETPGTSESATPESSGGSSGGSDKKYSIGEAIGTNSVGWAVITDEYKVP
SKKFKVEGNTDRHSIKKNEIGAEEFDSGETAEATREKRTARRRYTRRKNRICYEQEIFSNEMA
KVDDSFFHREEESFEVEEDKKHERHPIFGNIVDEVAYHEKYPTIYHERKKEVDSTDKADEREI
YEAEAHMIKFRGHFEIEGDENPDNSDVDKEFIQEVQTYNQEFEENPINASGVDAKAIESARES
KSRREENEIAQEPGEKKNGEFGNEIAESEGETPNFKSNFDEAEDAKEQESKDTYDDDEDNEEA
QIGDQYADEFEAAKNESDAIEESDIERVNTEITKAPESASMIKRYDEHHQDETEEKAEVRQQE
PEKYKEIFFDQSKNGYAGYIDGGASQEEFYKFIKPIEEKMDGTEEEEVKENREDEERKQRTFD
NGSIPHQIHEGEEHAIERRQEDFYPFEKDNREKIEKIETFRIPYYVGPEARGNSRFAWMTRKS
EETITPWNFEEWDKGASAQSFIERMTNFDKNLPNEKVLPKHSLLYEYFTVYNELTKVKYVTEG
MRKPAFESGEQKKAIVDEEFKTNRKVTVKQEKEDYFKKIECFDSVEISGVEDRFNASEGTYHD
EEKIIKDKDFEDNEENEDIEEDIVETETEFEDREMIEEREKTYAHEFDDKVMKQEKRRRYTGW
GRESRKEINGIRDKQSGKTIEDFEKSDGFANRNFMQEIHDDSETFKEDIQKAQVSGQGDSEHE
HIANEAGSPAIKKGIEQTVKWDEEVKVMGRHKPENIVIEMARENQTTQKGQKNSRERMKRIEE
GIKEEGSQIEKEHPVENTQEQNEKEYEYYEQNGRDMYVDQEEDINRESDYDVDHIVPQSFEKD
DSIDNKVETRSDKNRGKSDNVPSEEVVKKMKNYWRQEENAKEITQRKFDNETKAERGGESEED
KAGFIKRQEVETRQITKHVAQIEDSRMNTKYDENDKEIREVKVITEKSKEVSDFRKDFQFYKV
REINNYHHAHDAYENAVVGTAEIKKYPKEESEFVYGDYKVYDVRKMIAKSEQEIGKATAKYFF
YSNIMNFFKTEITEANGEIRKRPEIETNGETGEIVWDKGRDFATVRKVESMPQVNIVKKTEVQ
TGGFSKESIEPKRNSDKEIARKKDWDPKKYGGFVSPTVAYSVEVVAKVEKGKSKKEKSVKEEE
GITIMERSSFEKNPIDFEEAKGYKEVKKDEIIKEPKYSEFEEENGRKRMEASAREEQKGNEEA
EPSKYVNFEYEASHYEKEKGSPEDNEQKQEFVEQHKHYEDEIIEQISEFSKRVIEADANEDKV
ESAYNKHRDKPIREQAENIIHEFTETNEGAPAAFKYFDTTIDRKQYRSTKEVLDAILIHQSIT
GLYETRIDLSQLGGDSGGSKRTADGSEFEPKKKRKV
ABE8e(TadA-8e V106W)
(SEQ ID NO: 388)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGWRN
SKRGAAGSLMNVLNYPGMNHRVEITEGILADECAALLCDFYRMPRQVFNAQKKAQSSINSGGS
SGGSSGSETPGTSESATPESSGGSSGGSDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIANLAG
SPAIKKGILQTVKVVDELVKVMGRHKPENIVIEMARENQTTQKGQKNSRERMKRIEEGIKELG
SQILKEHPVENTQLQNEKLYLYYLQNGRDMYVDQELDINRLSDYDVDHIVPQSFLKDDSIDNK
VLTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKLKSVKELLGITIME
RSSFEKNPIDFLEAKGYKEVKKDLIIKLPKYSLFELENGRKRMLASAGELQKGNELALPSKYV
NFLYLASHYEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQSITGLYETR
IDLSQLGGDSGGSKRTADGSEFEPKKKRKV

In some embodiments, the adenine base editor comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 373-388, or to any of the base editors described herein. In some embodiments, the base editor comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NOs: 373-388, or any of the adenine base editors described herein. In some embodiments, the base editor comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1750, or at least 1800 identical contiguous amino acid residues as compared to any one of the amino acid sequences set forth in SEQ ID NOs: 373-388, or any of the adenine base editors provided herein. In some embodiments, the base editor (base editor) comprises the amino acid sequence of SEQ ID NO: 380, or a variant thereof that is at least 60%, at least 65%, at least 70%, at least 75%, at least 80%, at least 85%, at least 90%, at least 95%, at least 96%, at least 97%, at least 98%, at least 99%, or at least 99.5% identical.

In some embodiments, the base editors provided herein are capable of modifying a specific nucleotide base without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g. mutate or deaminate) a specific nucleotide within a nucleic acid, without generating a large number of insertions or deletions (i.e., indels) in the nucleic acid. In certain embodiments, any of the base editors described herein are capable of generating a greater proportion of intended modifications (e.g., point mutations or deaminations) versus indels. In some embodiments, the base editors described herein are capable of generating a ratio of intended point mutations to indels that is greater than 1:1. In some embodiments, the base editors described herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method. In some embodiments, to calculate indel frequencies, sequencing reads are scanned for exact matches to two 10-bp sequences that flank both sides of a window in which indels might occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively.

In some embodiments, the base editors described herein are capable of limiting formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors described herein are capable of limiting the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, an number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some aspects of the disclosure are based on the recognition that any of the base editors described herein are capable of efficiently generating an intended mutation, such as a point mutation, in a nucleic acid (e.g. a nucleic acid within a genome of a subject) without generating a significant number of unintended mutations, such as unintended point mutations. In some embodiments, an intended mutation is a mutation that is generated by a specific base editor bound to a gRNA, specifically designed to generate the intended mutation. In some embodiments, the intended mutation is a mutation associated with a disease or disorder. In some embodiments, the intended mutation is an adenine (A) to guanine (G) point mutation associated with a disease or disorder. In some embodiments, the intended mutation is a thymine (T) to cytosine (C) point mutation associated with a disease or disorder. In some embodiments, the intended mutation is an adenine (A) to guanine (G) point mutation within the coding region of a gene. In some embodiments, the intended mutation is a thymine (T) to cytosine (C) point mutation within the coding region of a gene. In some embodiments, the intended mutation is a point mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors described herein are capable of generating a ratio of intended mutations to unintended mutations (e.g., intended point mutations:unintended point mutations) that is greater than 1:1. In some embodiments, any of the base editors described herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described in the “Base Editor Efficiency” section, herein, may be applied to any of the base editors, or methods of using the base editors described herein.

### Exemplary CBEs

In some aspects, the methods of the present disclosure provides cytidine base editors (CBEs) comprising a napDNAbp domain and a cytosine deaminase domain that enzymatically deaminates a cytosine nucleobase of a C:G nucleobase pair to a uracil. The uracil may be subsequently converted to a thymine (T) by the cell's DNA repair and replication machinery. The mismatched guanine (G) on the opposite strand may subsequently be converted to an adenine (A) by the cell's DNA repair and replication machinery. In this manner, a target C:G nucleobase pair is ultimately converted to a T:A nucleobase pair.

In some aspects, the base editing methods of the disclosure comprise the use of an cytidine base editor. Exemplary cytidine base editors include, but are not limited to, BE3, BE3.9max, BE4max, BE4-SaKKH, BE3.9-NG, BE3.9-NRRH, BE4max-VQR, or BE4max-VRQR. In certain embodiments, the cytidine base editor used in the disclosed methods is a BE4max, BE4-SaKKH, BE4max-VQR, or BE4max-VRQR. Other CBEs may be used to deaminate a C nucleobase in accordance with the disclosed methods.

In some aspects, the disclosure provides complexes of base editors and guide RNAs that comprise a CBE. Exemplary cytidine base editors of the disclosed complexes include, but are not limited to, BE3, BE3.9max, BE4max, BE4-SaKKH, BE3.9-NG, BE3.9-NRRH, BE4max-VQR, or BE4max-VRQR. In certain embodiments, the cytidine base editor used in the disclosed complexes is a BE4max, BE4-SaKKH, BE4max-VQR, or BE4max-VRQR. Other CBEs may be used to deaminate a C nucleobase in accordance with the disclosed complexes.

Exemplary complexes of CBEs may provide an off-target editing frequency of less than 2.0% after being contacted with a nucleic acid molecule comprising a target sequence, e.g., a target nucleobase pair. Further exemplary CBE complexes provide an off-target editing frequency of less than 1.5% after being contacted with a nucleic acid molecule comprising a target sequence comprising a target nucleobase pair. Further exemplary CBE complexes may provide an off-target editing frequency of less than 1.25%, less than 1.1%, less than 1%, less than 0.75%, less than 0.5%, less than 0.4%, less than 0.25%, less than 0.2%, less than 0.15%, less than 0.1%, less than 0.05%, or less than 0.025%, after being contacted with a nucleic acid molecule comprising a target sequence.

For instance, the cytidine base editors YE1-BE4, YE1-CP1028, YE1-SpCas9-NG (also referred to herein as YE1-NG), R33A-BE4, and R33A+K34A-BE4-CP1028, which are described below, may exhibit off-target editing frequencies of less than 0.75% (e.g., about 0.4% or less) while maintaining on-target editing efficiencies of about 60% or more, in target sequences in mammalian cells. Each of these base editors comprises modified cytosine deaminases (e.g., YE1, R33A, or R33A+K34A) and may further comprise a modified napDNAbp domain such as a circularly permuted Cas9 domain (e.g., CP1028) or a Cas9 domain with an expanded PAM window (e.g., SpCas9-NG). These five base editors may be the most preferred for applications in which off-target editing, and in particular Cas9-independent off-target editing, must be minimized. In particular, base editors comprising a YE1 deaminase domain provide efficient on-target editing with greatly decreased Cas9-independent editing, as confirmed by whole-genome sequencing.

Exemplary CBEs may further possess an on-target editing efficiency of more than 50% after being contacted with a nucleic acid molecule comprising a target sequence. Further exemplary CBEs possess an on-target editing efficiency of more than 60% after being contacted with a nucleic acid molecule comprising a target sequence. Further exemplary CBEs possess an on-target editing efficiency of more than 65%, more than 70%, more than 75%, more than 80%, more than 82.5%, or more than 85% after being contacted with a nucleic acid molecule comprising a target sequence. The disclosed CBEs may exhibit indel frequencies of less than 0.75%, less than 0.6%, less than 0.5%, less than 0.4%, less than 0.3%, or less than 0.2% after being contacted with a nucleic acid molecule containing a target sequence.

The disclosed CBEs may further comprise one or more nuclear localization signals (NLSs) and/or two or more uracil glycosylase inhibitor (UGI) domains. Thus, the base editors may comprise the structure: NH₂-[first nuclear localization sequence]-[cytosine deaminase domain]-[napDNAbp domain]-[first UGI domain]-[second UGI domain]-[second nuclear localization sequence]-COOH, wherein each instance of “]-[” indicates the presence of an optional linker sequence. Exemplary CBEs may have a structure that comprises the “BE4max” architecture, with an NH₂-[NLS]-[cytosine deaminase]-[Cas9 nickase]-[UGI domain]-[UGI domain]-[NLS]-COOH structure, having optimized nuclear localization signals and wherein the napDNAbp domain comprises a Cas9 nickase. This BE4max structure was reported to have optimized codon usage for expression in human cells, as reported in Koblan et al., Nat Biotechnol. 2018; 36(9):843-846, herein incorporated by reference.

In other embodiments, exemplary CBEs may have a structure that comprises a modified BE4max architecture that contains a napDNAbp domain comprising a Cas9 variant other than Cas9 nickase, such as SpCas9-NG, xCas9, or circular permutant CP1028. Accordingly, exemplary CBEs may comprise the structure: NH₂-[NLS]-[cytosine deaminase]-[xCas9]-[UGI domain]-[UGI domain][NLS]-COOH; or NH₂-[NLS]cytosine deaminase]-[SpCas9-NG]-[UGI domain]-[UGI domain][NLS]-COOH, wherein each instance of “]-[” indicates the presence of an optional linker sequence.

The disclosed CBEs may comprise modified (or evolved) cytosine deaminase domains, such as deaminase domains that recognize an expanded PAM sequence, have improved efficiency of deaminating 5′-GC targets, and/or make edits in a narrower target window, In some embodiments, the disclosed cytidine base editors comprise evolved nucleic acid programmable DNA binding proteins (napDNAbp), such as an evolved Cas9.

In some embodiments, exemplary CBEs for use in the present disclosure include those from Gaudelli et al. Nat. Commun. (2020) 11:2052, incorporated by reference herein. These base editors include, but are not limited to, a base editor comprising a sequence that is at least 90%, at least 95%, at least 98%, at least 99%, or at least 99.5% identical to the sequences reported therein for next-generation BE4 mutants BE4-PpAPOBEC1, BE4-PpAPOBEC1 H122A, BE4-PpAPOBEC1 R33A, BE4-RrA3F, BE4-RrA3F F130L, BE4-AmAPOBEC1, BE4-SsAPOBEC3B, and BE4-SsAPOBEC3B R54Q.

In some embodiments, exemplary CBEs for use in the present disclosure include those provided in Gehrke, et al. An APOBEC3A-Cas9 base editor with minimized bystander and off-target activities, Nat. Biotechnol. 36(10), 977-982 (2018); and Lee et al. Single C-to-T substitution using engineered APOBEC3G-nCas9 base editors with minimum genome- and transcriptome-wide off-target effects, Science Advances 6, eaba1773 (2020), each of which are incorporated by reference herein. Gehrke et al. provides APOBEC3A-BE3 variants having N57G, N57A, and N57Q/Y130F mutations in the APOBEC3A deaminase domain; and Lee et al. provides APOBEC3F-nCas9 variants (A3G-BE4.4, A3G-BE5.13, and A3G-BE5.14), which contain mutations in the APOBEC3G deaminase domain and exhibit high editing efficiencies and precision in the context of a CC target sequence motif.

In some embodiments, exemplary CBEs for use in the present disclosure include those provided in WO 2020/051562, published Mar. 12, 2020, which discloses multi-effector cytosine base editors having two napDNAbp domains (dCas9 and Cas9n), cytidine deaminase domain and and a UGI domain. In some embodiments, exemplary CBEs for use in the present disclosure include those provided in WO 2020/028823, published Feb. 6, 2020, which discloses multi-effector base editors having a pmCDA cytidine deaminase domain, a TadA7.10 adenosine deaminase domain, a UGI domain, and a Cas9n domain.

Exemplary cytidine base editors comprise sequences that are at least least 85%, at least 90%, at least 95%, at least 98%, at least 99%, or at least 99.5% identical to the following amino acid sequences, SEQ ID NOs: 350-372.

Where indicated, “BE4-” and “-BE4” refer to the BE4max architecture, or NH₂-[first nuclear localization sequence]-[cytosine deaminase domain]-[32aa linker]-[SpCas9 nickase (nCas9, or nSpCas9) domain]-[9aa linker]-[first UGI domain]-[9aa-linker]-[second UGI domain]-[second nuclear localization sequence]-COOH. Where indicated, “BE4max, modified with SpCas9-NG” and “-SpCas9-NG” refer to a modified BE4max architecture in which the SpCas9 nickase domain has been replaced with an SpCas9-NG, i.e., NH₂-[first nuclear localization sequence]-[cytosine deaminase domain]-[32aa linker]-[SpCas9-NG]-[9aa linker]-[first UGI domain]-[9aa-linker]-[second UGI domain]-[second nuclear localization sequence]-COOH.

As discussed above, preferred base editors comprise modified cytosine deaminases (e.g., YE1, R33A, or R33A+K34A) and may further comprise a modified napDNAbp domain such as a Cas9 domain with an expanded PAM window (e.g., SpCas9-NG). The napDNAbp domains in the following amino acid sequences are indicated in italics.

BE4max
(SEQ ID NO: 350)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHWPRYP
HLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVLGN
TDRHSIKKNLIGALLFDSGETAEATRLKRTARRRYTRRKNRICYEQEIFSNEMAKVDDSFFHR
EEESFEVEEDKKHERHPIFGNIVDEVAYHEKYPTIYHERKKEVDSTDKADEREIYEAEAHMIK
FRGHFEIEGDENPDNSDVDKEFIQEVQTYNQEFEENPINASGVDAKAIESARESKSRREENEI
AQEPGEKKNGEFGNEIAESEGETPNFKSNFDEAEDAKEQESKDTYDDDEDNEEAQIGDQYADE
FEAAKNESDAIEESDIERVNTEITKAPESASMIKRYDEHHQDETEEKAEVRQQEPEKYKEIFF
DQSKNGYAGYIDGGASQEEFYKFIKPIEEKMDGTEEEEVKENREDEERKQRTFDNGSIPHQIH
EGEEHAIERRQEDFYPFEKDNREKIEKIETFRIPYYVGPEARGNSRFAWMTRKSEETITPWNF
EEVVDKGASAQSFIERMTNFDKNEPNEKVEPKHSEEYEYFTVYNEETKVKYVTEGMRKPAFES
GEQKKAIVDEEFKTNRKVTVKQEKEDYFKKIECFDSVEISGVEDRFNASEGTYHDEEKIIKDK
DFEDNEENEDIEEDIVETETEFEDREMIEERLKTYAHLFDDKVMKQLKRRRYTGWGRLSRKLI
NGIRDKQSGKTILDFLKSDGFANRNFMQEIHDDSETFKEDIQKAQVSGQGDSEHEHIANEAGS
PAIKKGIEQTVKVVDEEVKVMGRHKPENIVIEMARENQTTQKGQKNSRERMKRIEEGIKEEGS
QIEKEHPVENTQEQNEKEYEYYEQNGRDMYVDQEEDINRESDYDVDHIVPQSFEKDDSIDNKV
ETRSDKNRGKSDNVPSEEWKKMKNYWRQEENAKEITQRKFDNETKAERGGESEEDKAGFIKRQ
EVETRQITKHVAQILDSRMNTKYDENDKLIREVKVITLKSKLVSDFRKDFQFYKVREINNYHH
AHDAYLNAVVGTAEIKKYPKEESEFVYGDYKVYDVRKMIAKSEQEIGKATAKYFFYSNIMNFF
KTEITEANGEIRKRPEIETNGETGEIVWDKGRDFATVRKVESMPQVNIVKKTEVQTGGFSKES
IEPKRNSDKEIARKKDWDPKKYGGFDSPTVAYSVEWAKVEKGKSKKEKSVKEEEGITIMERSS
FEKNPIDFEEAKGYKEVKKDEIIKEPKYSEFEEENGRKRMEASAGEEQKGNEEAEPSKYVNFE
YEASHYEKEKGSPEDNEQKQEFVEQHKHYEDEIIEQISEFSKRVIEADANEDKVESAYNKHRD
KPIREQAENIIHEFTETNEGAPAAFKYFDTTIDRKRYTSTKEVEDATEIHQSITGEYETRIDL
SQLGGDSGGSGGSGGSTNLSDIIEKETGKQLVIQESILMLPEEVEEVIGNKPESDILVHTAYD
ESTDENVMLLTSDAPEYKPWALVIQDSNGENKIKMLSGGSGGSGGSTNLSDIIEKETGKQLVI
QESILMLPEEVEEVIGNKPESDILVHTAYDESTDENVMLLTSDAPEYKPWALVIQDSNGENKI
KMLSGGSKRTADGSEFEPKKKRKV
YE1-BE4
(SEQ ID NO: 351)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSYSPCGECSRAITEFLSRYP
HVTLFIYIARLYHHADPENRQGLRDLISSGVTIQIMTEQESGYCWRNFVNYSPSNEAHWPRYP
HLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVEGN
TDRHSIKKNEIGAEEFDSGETAEATRLKRTARRRYTRRKNRICYEQEIFSNEMAKVDDSFFHR
EEESFEVEEDKKHERHPIFGNIVDEVAYHEKYPTIYHERKKEVDSTDKADEREIYEAEAHMIK
FRGHFEIEGDENPDNSDVDKEFIQEVQTYNQEFEENPINASGVDAKAIESARESKSRREENEI
AQEPGEKKNGEFGNEIAESEGETPNFKSNFDEAEDAKEQESKDTYDDDEDNEEAQIGDQYADE
FEAAKNESDAIEESDIERVNTEITKAPESASMIKRYDEHHQDETEEKAEVRQQEPEKYKEIFF
DQSKNGYAGYIDGGASQEEFYKFIKPIEEKMDGTEEEEVKENREDEERKQRTFDNGSIPHQIH
EGEEHAIERRQEDFYPFEKDNREKIEKIETFRIPYYVGPEARGNSRFAWMTRKSEETITPWNF
EEVVDKGASAQSFIERMTNFDKNEPNEKVEPKHSEEYEYFTVYNEETKVKYVTEGMRKPAFES
GEQKKAIVDEEFKTNRKVTVKQEKEDYFKKIECFDSVEISGVEDRFNASEGTYHDEEKIIKDK
DFEDNEENEDIEEDIVETETEFEDREMIEEREKTYAHEFDDKVMKQEKRRRYTGWGRESRKEI
NGIRDKQSGKTIEDFEKSDGFANRNFMQEIHDDSETFKEDIQKAQVSGQGDSEHEHIANEAGS
PAIKKGIEQTVKVVDEEVKVMGRHKPENIVIEMARENQTTQKGQKNSRERMKRIEEGIKELGS
QILKEHPVENTQLQNEKEYEYYEQNGRDMYVDQEEDINRESDYDVDHIVPQSFEKDDSIDNKV
ETRSDKNRGKSDNVPSEEWKKMKNYWRQEENAKEITQRKFDNETKAERGGESEEDKAGFIKRQ
EVETRQITKHVAQILDSRMNTKYDENDKLIREVKVITLKSKLVSDFRKDFQFYKVREINNYHH
AHDAYLNAVVGTAEIKKYPKEESEFVYGDYKVYDVRKMIAKSEQEIGKATAKYFFYSNIMNFF
KTEITEANGEIRKRPEIETNGETGEIVWDKGRDFATVRKVESMPQVNIVKKTEVQTGGFSKES
IEPKRNSDKEIARKKDWDPKKYGGFDSPTVAYSVEWAKVEKGKSKKEKSVKEEEGITIMERSS
FEKNPIDFEEAKGYKEVKKDEIIKEPKYSEFEEENGRKRMEASAGEEQKGNEEAEPSKYVNFE
YEASHYEKEKGSPEDNEQKQEFVEQHKHYEDEIIEQISEFSKRVIEADANEDKVESAYNKHRD
KPIREQAENIIHEFTETNEGAPAAFKYFDTTIDRKRYTSTKEVEDATEIHQSITGEYETRIDL
SQLGGDSGGSGGSGGSTNLSDIIEKETGKQLVIQESILMLPEEVEEVIGNKPESDILVHTAYD
ESTDENVMLLTSDAPEYKPWALVIQDSNGENKIKMLSGGSGGSGGSTNLSDIIEKETGKQLVI
QESILMLPEEVEEVIGNKPESDILVHTAYDESTDENVMLLTSDAPEYKPWALVIQDSNGENKI
KMLSGGSKRTADGSEFEPKKKRKV
YE2-BE4
(SEQ ID NO: 352)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSYSPCGECSRAITEFLSRYP
HVTLFIYIARLYHHADPRNRQGLEDLISSGVTIQIMTEQESGYCWRNFVNYSPSNEAHWPRYP
HLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVEGN
TDRHSIKKNEIGAEEFDSGETAEATRLKRTARRRYTRRKNRICYEQEIFSNEMAKVDDSFFHR
EEESFEVEEDKKHERHPIFGNIVDEVAYHEKYPTIYHERKKEVDSTDKADEREIYEAEAHMIK
FRGHFEIEGDENPDNSDVDKEFIQEVQTYNQEFEENPINASGVDAKAIESARESKSRREENEI
AQEPGEKKNGEFGNEIAESEGETPNFKSNFDEAEDAKEQESKDTYDDDEDNEEAQIGDQYADE
FEAAKNESDAIEESDIERVNTEITKAPESASMIKRYDEHHQDETEEKAEVRQQEPEKYKEIFF
DQSKNGYAGYIDGGASQEEFYKFIKPIEEKMDGTEEEEVKENREDEERKQRTFDNGSIPHQIH
EGEEHAIERRQEDFYPFEKDNREKIEKIETFRIPYYVGPEARGNSRFAWMTRKSEETITPWNF
EEVVDKGASAQSFIERMTNFDKNEPNEKVEPKHSEEYEYFTVYNEETKVKYVTEGMRKPAFES
GEQKKAIVDEEFKTNRKVTVKQEKEDYFKKIECFDSVEISGVEDRFNASEGTYHDEEKIIKDK
DFEDNEENEDIEEDIVETETEFEDREMIEEREKTYAHEFDDKVMKQEKRRRYTGWGRESRKEI
NGIRDKQSGKTIEDFEKSDGFANRNFMQEIHDDSETFKEDIQKAQVSGQGDSEHEHIANEAGS
PAIKKGIEQTVKVVDEEVKVMGRHKPENIVIEMARENQTTQKGQKNSRERMKRIEEGIKEEGS
QIEKEHPVENTQEQNEKEYEYYEQNGRDMYVDQEEDINRESDYDVDHIVPQSFEKDDSIDNKV
ETRSDKNRGKSDNVPSEEWKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREINNYHH
AHDAYLNAVVGTAEIKKYPKEESEFVYGDYKVYDVRKMIAKSEQEIGKATAKYFFYSNIMNFF
KTEITEANGEIRKRPEIETNGETGEIVWDKGRDFATVRKVESMPQVNIVKKTEVQTGGFSKES
IEPKRNSDKEIARKKDWDPKKYGGFDSPTVAYSVEWAKVEKGKSKKEKSVKEEEGITIMERSS
FEKNPIDFEEAKGYKEVKKDEIIKEPKYSEFEEENGRKRMEASAGEEQKGNEEAEPSKYVNFE
YEASHYEKEKGSPEDNEQKQEFVEQHKHYEDEIIEQISEFSKRVIEADANEDKVESAYNKHRD
KPIREQAENIIHEFTETNEGAPAAFKYFDTTIDRKRYTSTKEVEDATEIHQSITGEYETRIDL
SQLGGDSGGSGGSGGSTNLSDIIEKETGKQLVIQESILMLPEEVEEVIGNKPESDILVHTAYD
ESTDENVMLLTSDAPEYKPWALVIQDSNGENKIKMLSGGSGGSGGSTNLSDIIEKETGKQLVI
QESILMLPEEVEEVIGNKPESDILVHTAYDESTDENVMLLTSDAPEYKPWALVIQDSNGENKI
KMLSGGSKRTADGSEFEPKKKRKV
YEE-BE4
(SEQ ID NO: 353)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSYSPCGECSRAITEFLSRYP
HVTLFIYIARLYHHADPENRQGLEDLISSGVTIQIMTEQESGYCWRNFVNYSPSNEAHWPRYP
HLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVEGN
TDRHSIKKNEIGAEEFDSGETAEATRLKRTARRRYTRRKNRICYEQEIFSNEMAKVDDSFFHR
EEESFEVEEDKKHERHPIFGNIVDEVAYHEKYPTIYHERKKEVDSTDKADEREIYEAEAHMIK
FRGHFEIEGDENPDNSDVDKEFIQEVQTYNQEFEENPINASGVDAKAIESARESKSRREENEI
AQEPGEKKNGEFGNEIAESEGETPNFKSNFDEAEDAKEQESKDTYDDDEDNEEAQIGDQYADE
FEAAKNESDAIEESDIERVNTEITKAPESASMIKRYDEHHQDETEEKAEVRQQEPEKYKEIFF
DQSKNGYAGYIDGGASQEEFYKFIKPIEEKMDGTEEEEVKENREDEERKQRTFDNGSIPHQIH
EGEEHAIERRQEDFYPFEKDNREKIEKIETFRIPYYVGPEARGNSRFAWMTRKSEETITPWNF
EEVVDKGASAQSFIERMTNFDKNEPNEKVEPKHSEEYEYFTVYNEETKVKYVTEGMRKPAFES
GEQKKAIVDEEFKTNRKVTVKQEKEDYFKKIECFDSVEISGVEDRFNASEGTYHDEEKIIKDK
DFEDNEENEDIEEDIVETETEFEDREMIEEREKTYAHEFDDKVMKQEKRRRYTGWGRESRKEI
NGIRDKQSGKTIEDFEKSDGFANRNFMQEIHDDSETFKEDIQKAQVSGQGDSEHEHIANEAGS
PAIKKGIEQTVKVVDEEVKVMGRHKPENIVIEMARENQTTQKGQKNSRERMKRIEEGIKEEGS
QIEKEHPVENTQEQNEKEYEYYEQNGRDMYVDQEEDINRESDYDVDHIVPQSFEKDDSIDNKV
ETRSDKNRGKSDNVPSEEWKKMKNYWRQEENAKEITQRKFDNETKAERGGESEEDKAGFIKRQ
EVETRQITKHVAQIEDSRMNTKYDENDKEIREVKVITEKSKEVSDFRKDFQFYKVREINNYHH
AHDAYENAVVGTALIKKYPKLESEFVYGDYKVYDVRKMIAKSEQEIGKATAKYFFYSNIMNFF
KTEITLANGEIRKRPEIETNGETGEIVWDKGRDFATVRKVESMPQVNIVKKTEVQTGGFSKES
IEPKRNSDKEIARKKDWDPKKYGGFDSPTVAYSVEWAKVEKGKSKKEKSVKEEEGITIMERSS
FEKNPIDFEEAKGYKEVKKDEIIKEPKYSEFEEENGRKRMEASAGEEQKGNEEAEPSKYVNFE
YEASHYEKEKGSPEDNEQKQEFVEQHKHYEDEIIEQISEFSKRVIEADANEDKVESAYNKHRD
KPIREQAENIIHEFTETNEGAPAAFKYFDTTIDRKRYTSTKEVEDATEIHQSITGEYETRIDL
SQLGGDSGGSGGSGGSTNLSDIIEKETGKQLVIQESILMLPEEVEEVIGNKPESDILVHTAYD
ESTDENVMLLTSDAPEYKPWALVIQDSNGENKIKMLSGGSGGSGGSTNLSDIIEKETGKQLVI
QESILMLPEEVEEVIGNKPESDILVHTAYDESTDENVMLLTSDAPEYKPWALVIQDSNGENKI
KMLSGGSKRTADGSEFEPKKKRKV
EE-BE4
(SEQ ID NO: 354)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSWSPCGECSRAITEFLSRYP
HVTLFIYIARLYHHADPENRQGLEDLISSGVTIQIMTEQESGYCWRNFVNYSPSNEAHWPRYP
HLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVEGN
TDRHSIKKNEIGAEEFDSGETAEATRLKRTARRRYTRRKNRICYEQEIFSNEMAKVDDSFFHR
EEESFEVEEDKKHERHPIFGNIVDEVAYHEKYPTIYHERKKEVDSTDKADEREIYEAEAHMIK
FRGHFEIEGDENPDNSDVDKEFIQEVQTYNQEFEENPINASGVDAKAIESARESKSRREENEI
AQEPGEKKNGEFGNEIAESEGETPNFKSNFDEAEDAKEQESKDTYDDDEDNEEAQIGDQYADE
FEAAKNESDAIEESDIERVNTEITKAPESASMIKRYDEHHQDETEEKAEVRQQEPEKYKEIFF
DQSKNGYAGYIDGGASQEEFYKFIKPIEEKMDGTEEEEVKENREDEERKQRTFDNGSIPHQIH
EGEEHAIERRQEDFYPFEKDNREKIEKIETFRIPYYVGPEARGNSRFAWMTRKSEETITPWNF
EEVVDKGASAQSFIERMTNFDKNEPNEKVEPKHSEEYEYFTVYNEETKVKYVTEGMRKPAFES
GEQKKAIVDEEFKTNRKVTVKQEKEDYFKKIECFDSVEISGVEDRFNASEGTYHDEEKIIKDK
DFEDNEENEDIEEDIVETETEFEDREMIEEREKTYAHEFDDKVMKQEKRRRYTGWGRESRKEI
NGIRDKQSGKTIEDFEKSDGFANRNFMQEIHDDSETFKEDIQKAQVSGQGDSEHEHIANEAGS
PAIKKGIEQTVKVVDEEVKVMGRHKPENIVIEMARENQTTQKGQKNSRERMKRIEEGIKEEGS
QIEKEHPVENTQEQNEKEYEYYEQNGRDMYVDQEEDINRESDYDVDHIVPQSFEKDDSIDNKV
ETRSDKNRGKSDNVPSEEWKKMKNYWRQEENAKEITQRKFDNETKAERGGESEEDKAGFIKRQ
EVETRQITKHVAQILDSRMNTKYDENDKLIREVKVITLKSKLVSDFRKDFQFYKVREINNYHH
AHDAYLNAVVGTAEIKKYPKEESEFVYGDYKVYDVRKMIAKSEQEIGKATAKYFFYSNIMNFF
KTEITEANGEIRKRPEIETNGETGEIVWDKGRDFATVRKVESMPQVNIVKKTEVQTGGFSKES
IEPKRNSDKLIARKKDWDPKKYGGFDSPTVAYSVLWAKVEKGKSKKLKSVKELLGITIMERSS
FEKNPIDFEEAKGYKEVKKDEIIKEPKYSEFEEENGRKRMEASAGEEQKGNEEAEPSKYVNFE
YEASHYEKEKGSPEDNEQKQEFVEQHKHYEDEIIEQISEFSKRVIEADANEDKVESAYNKHRD
KPIREQAENIIHEFTETNEGAPAAFKYFDTTIDRKRYTSTKEVEDATEIHQSITGEYETRIDL
SQLGGDSGGSGGSGGSTNLSDIIEKETGKQLVIQESILMLPEEVEEVIGNKPESDILVHTAYD
ESTDENVMLLTSDAPEYKPWALVIQDSNGENKIKMLSGGSGGSGGSTNLSDIIEKETGKQLVI
QESILMLPEEVEEVIGNKPESDILVHTAYDESTDENVMLLTSDAPEYKPWALVIQDSNGENKI
KMLSGGSKRTADGSEFEPKKKRKV
R33A-BE4
(SEQ ID NO: 355)
MKRTADGSEFESPKKKRKVSSETGPVAVDPTLRRRIEPHEFEVFFDPRELAKETCLLYEINWG
GRHSIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHWPRYP
HLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVEGN
TDRHSIKKNEIGAEEFDSGETAEATRLKRTARRRYTRRKNRICYEQEIFSNEMAKVDDSFFHR
EEESFEVEEDKKHERHPIFGNIVDEVAYHEKYPTIYHERKKEVDSTDKADEREIYEAEAHMIK
FRGHFEIEGDENPDNSDVDKEFIQEVQTYNQEFEENPINASGVDAKAIESARESKSRREENEI
AQEPGEKKNGEFGNEIAESEGETPNFKSNFDEAEDAKEQESKDTYDDDEDNEEAQIGDQYADE
FEAAKNESDAIEESDIERVNTEITKAPESASMIKRYDEHHQDETEEKAEVRQQEPEKYKEIFF
DQSKNGYAGYIDGGASQEEFYKFIKPIEEKMDGTEEEEVKENREDEERKQRTFDNGSIPHQIH
EGEEHAIERRQEDFYPFEKDNREKIEKIETFRIPYYVGPEARGNSRFAWMTRKSEETITPWNF
EEVVDKGASAQSFIERMTNFDKNEPNEKVEPKHSEEYEYFTVYNEETKVKYVTEGMRKPAFES
GEQKKAIVDEEFKTNRKVTVKQEKEDYFKKIECFDSVEISGVEDRFNASEGTYHDEEKIIKDK
DFEDNEENEDIEEDIVETETEFEDREMIEEREKTYAHEFDDKVMKQEKRRRYTGWGRESRKEI
NGIRDKQSGKTIEDFEKSDGFANRNFMQEIHDDSETFKEDIQKAQVSGQGDSEHEHIANEAGS
PAIKKGIEQTVKVVDEEVKVMGRHKPENIVIEMARENQTTQKGQKNSRERMKRIEEGIKEEGS
QIEKEHPVENTQEQNEKEYEYYEQNGRDMYVDQEEDINRESDYDVDHIVPQSFEKDDSIDNKV
ETRSDKNRGKSDNVPSEEWKKMKNYWRQEENAKEITQRKFDNETKAERGGESEEDKAGFIKRQ
EVETRQITKHVAQILDSRMNTKYDENDKLIREVKVITLKSKLVSDFRKDFQFYKVREINNYHH
AHDAYLNAVVGTAEIKKYPKEESEFVYGDYKVYDVRKMIAKSEQEIGKATAKYFFYSNIMNFF
KTEITEANGEIRKRPEIETNGETGEIVWDKGRDFATVRKVESMPQVNIVKKTEVQTGGFSKES
IEPKRNSDKEIARKKDWDPKKYGGFDSPTVAYSVEWAKVEKGKSKKEKSVKEEEGITIMERSS
FEKNPIDFEEAKGYKEVKKDEIIKEPKYSEFEEENGRKRMEASAGEEQKGNEEAEPSKYVNFL
YLASHYEKLKGSPEDNEQKQLFVEQHKHYLDEIIEQISEFSKRVILADANLDKVLSAYNKHRD
KPIREQAENIIHEFTETNEGAPAAFKYFDTTIDRKRYTSTKEVEDATEIHQSITGEYETRIDL
SQLGGDSGGSGGSGGSTNLSDIIEKETGKQLVIQESILMLPEEVEEVIGNKPESDILVHTAYD
ESTDENVMLLTSDAPEYKPWALVIQDSNGENKIKMLSGGSGGSGGSTNLSDIIEKETGKQLVI
QESILMLPEEVEEVIGNKPESDILVHTAYDESTDENVMLLTSDAPEYKPWALVIQDSNGENKI
KMLSGGSKRTADGSEFEPKKKRKV
R33A + K34A-BE4
(SEQ ID NO: 356)
MKRTADGSEFESPKKKRKVSSETGPVAVDPTLRRRIEPHEFEVFFDPRELAAETCLLYEINWG
GRHSIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHWPRYP
HLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVEGN
TDRHSIKKNEIGAEEFDSGETAEATRLKRTARRRYTRRKNRICYEQEIFSNEMAKVDDSFFHR
EEESFEVEEDKKHERHPIFGNIVDEVAYHEKYPTIYHERKKEVDSTDKADEREIYEAEAHMIK
FRGHFEIEGDENPDNSDVDKEFIQEVQTYNQEFEENPINASGVDAKAIESARESKSRREENEI
AQEPGEKKNGEFGNEIAESEGETPNFKSNFDEAEDAKEQESKDTYDDDEDNEEAQIGDQYADE
FEAAKNESDAIEESDIERVNTEITKAPESASMIKRYDEHHQDETEEKAEVRQQEPEKYKEIFF
DQSKNGYAGYIDGGASQEEFYKFIKPIEEKMDGTEEEEVKENREDEERKQRTFDNGSIPHQIH
EGEEHAIERRQEDFYPFEKDNREKIEKIETFRIPYYVGPEARGNSRFAWMTRKSEETITPWNF
EEVVDKGASAQSFIERMTNFDKNEPNEKVEPKHSEEYEYFTVYNEETKVKYVTEGMRKPAFES
GEQKKAIVDEEFKTNRKVTVKQEKEDYFKKIECFDSVEISGVEDRFNASEGTYHDEEKIIKDK
DFEDNEENEDIEEDIVETETEFEDREMIEEREKTYAHEFDDKVMKQEKRRRYTGWGRESRKEI
NGIRDKQSGKTIEDFEKSDGFANRNFMQEIHDDSETFKEDIQKAQVSGQGDSEHEHIANEAGS
PAIKKGIEQTVKVVDEEVKVMGRHKPENIVIEMARENQTTQKGQKNSRERMKRIEEGIKEEGS
QIEKEHPVENTQEQNEKEYEYYEQNGRDMYVDQEEDINRESDYDVDHIVPQSFEKDDSIDNKV
ETRSDKNRGKSDNVPSEEWKKMKNYWRQEENAKEITQRKFDNETKAERGGESEEDKAGFIKRQ
EVETRQITKHVAQILDSRMNTKYDENDKLIREVKVITLKSKLVSDFRKDFQFYKVREINNYHH
AHDAYLNAVVGTAEIKKYPKEESEFVYGDYKVYDVRKMIAKSEQEIGKATAKYFFYSNIMNFF
KTEITEANGEIRKRPEIETNGETGEIVWDKGRDFATVRKVESMPQVNIVKKTEVQTGGFSKES
IEPKRNSDKEIARKKDWDPKKYGGFDSPTVAYSVEWAKVEKGKSKKEKSVKEEEGITIMERSS
FEKNPIDFEEAKGYKEVKKDEIIKEPKYSEFEEENGRKRMEASAGEEQKGNEEAEPSKYVNFE
YEASHYEKEKGSPEDNEQKQEFVEQHKHYEDEIIEQISEFSKRVIEADANEDKVESAYNKHRD
KPIREQAENIIHEFTETNEGAPAAFKYFDTTIDRKRYTSTKEVEDATEIHQSITGEYETRIDL
SQLGGDSGGSGGSGGSTNLSDIIEKETGKQLVIQESILMLPEEVEEVIGNKPESDILVHTAYD
ESTDENVMLLTSDAPEYKPWALVIQDSNGENKIKMLSGGSGGSGGSTNLSDIIEKETGKQLVI
QESILMLPEEVEEVIGNKPESDILVHTAYDESTDENVMLLTSDAPEYKPWALVIQDSNGENKI
KMLSGGSKRTADGSEFEPKKKRKV
APOBEC3A(A3A)-BE4
(SEQ ID NO: 357)
MKRTADGSEFESPKKKRKVSEASPASGPRHLMDPHIFTSNFNNGIGRHKTYLCYEVERLDNGT
SVKMDQHRGFLHNQAKNLLCGFYGRHAELRFLDLVPSLQLDPAQIYRVTWFISWSPCFSWGCA
GEVRAFLQENTHVRLRIFAARIYDYDPLYKEALQMLRDAGAQVSIMTYDEFKHCWDTFVDHQG
CPFQPWDGLDEHSQALSGRLRAILQNQGNSGGSSGGSSGSEAPGASESAAPESSGGSSGGSDK
KYSIGLAIGTNSVGWAVITDEYKVPSKKFKVLGNTDRHSIKKNLIGALLFDSGETAEATRLKR
TARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSN
FDLAEDAKLQLSKDTYDDDLDNLLAQIGDQYADLFLAAKNLSDAILLSDILRVNTEITKAPLS
ASMIKRYDEHHQDLTLLKALVRQQLPEKYKEIFFDQSKNGYAGYIDGGASQEEFYKFIKPILE
KMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFANRNFMQL
IHDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEWKKMKNYWRQL
LNAKLITQRKFDNLTKAERGGLSELDKAGFIKRQLVETRQITKHVAQILDSRMNTKYDENDKL
IREVKVITLKSKLVSDFRKDFQFYKVREINNYHHAHDAYLNAVVGTALIKKYPKLESEFVYGD
YKVYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGGFDSPT
VAYSVLWAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLFVEQHKHY
LDEIIEQISEFSKRVILADANLDKVLSAYNKHRDKPIREQAENIIHLFTLTNLGAPAAFKYFD
TTIDRKRYTSTKEVLDATLIHQSITGLYETRIDLSQLGGDSGGSGGSGGSTNLSDIIEKETGK
QLVIQESILMLPEEVEEVIGNKPESDILVHTAYDESTDENVMLLTSDAPEYKPWALVIQDSNG
ENKIKMLSGGSGGSGGSTNLSDIIEKETGKQLVIQESILMLPEEVEEVIGNKPESDILVHTAY
DESTDENVMLLTSDAPEYKPWALVIQDSNGENKIKMLSGGSKRTADGSEFEPKKKRKV
APOBEC3B(A3B)-BE4
(SEQ ID NO: 358)
MKRTADGSEFESPKKKRKVNPQIRNPMERMYRDTFYDNFENEPILYGRSYTWLCYEVKIKRGR
SNLLWDTGVFRGQVYFKPQYHAEMCFLSWFCGNQLPAYKCFQITWFVSWTPCPDCVAKLAEFL
SEHPNVTLTISAARLYYYWERDYRRALCRLSQAGARVTIMDYEEFAYCWENFVYNEGQQFMPW
YKFDENYAFLHRTLKEILRYLMDPDTFTFNFNNDPLVLRRRQTYLCYEVERLDNGTWVLMDQH
MGFLCNEAKNLLCGFYGRHAELRFLDLVPSLQLDPAQIYRVTWFISWSPCFSWGCAGEVRAFL
QENTHVRLRIFAARIYDYDPLYKEALQMLRDAGAQVSIMTYDEFEYCWDTFVYRQGCPFQPWD
GLEEHSQALSGRLRAILQNQGNSGGSSGGSSGSETPGTSESATPESSGGSSGGSDKKYSIGLA
IGTNSVGWAVITDEYKVPSKKFKVEGNTDRHSIKKNEIGAEEFDSGETAEATREKRTARRRYT
RRKNRICYEQEIFSNEMAKVDDSFFHREEESFEVEEDKKHERHPIFGNIVDEVAYHEKYPTIY
HERKKEVDSTDKADEREIYEAEAHMIKFRGHFEIEGDENPDNSDVDKEFIQEVQTYNQEFEEN
PINASGVDAKAIESARESKSRREENEIAQEPGEKKNGEFGNEIAESEGETPNFKSNFDEAEDA
KEQESKDTYDDDEDNEEAQIGDQYADEFEAAKNESDAIEESDIERVNTEITKAPESASMIKRY
DEHHQDETEEKAEVRQQEPEKYKEIFFDQSKNGYAGYIDGGASQEEFYKFIKPIEEKMDGTEE
EEVKENREDEERKQRTFDNGSIPHQIHEGEEHAIERRQEDFYPFEKDNREKIEKIETFRIPYY
VGPEARGNSRFAWMTRKSEETITPWNFEEWDKGASAQSFIERMTNFDKNEPNEKVEPKHSEEY
EYFTVYNEETKVKYVTEGMRKPAFESGEQKKAIVDEEFKTNRKVTVKQEKEDYFKKIECFDSV
EISGVEDRFNASEGTYHDEEKIIKDKDFEDNEENEDIEEDIVETETEFEDREMIEEREKTYAH
EFDDKVMKQEKRRRYTGWGRESRKEINGIRDKQSGKTIEDFEKSDGFANRNFMQEIHDDSETF
KEDIQKAQVSGQGDSEHEHIANEAGSPAIKKGIEQTVKVVDEEVKVMGRHKPENIVIEMAREN
QTTQKGQKNSRERMKRIEEGIKEEGSQIEKEHPVENTQEQNEKEYEYYEQNGRDMYVDQEEDI
NRESDYDVDHIVPQSFEKDDSIDNKVETRSDKNRGKSDNVPSEEVVKKMKNYWRQEENAKEIT
QRKFDNETKAERGGESEEDKAGFIKRQEVETRQITKHVAQIEDSRMNTKYDENDKEIREVKVI
TEKSKEVSDFRKDFQFYKVREINNYHHAHDAYENAWGTALIKKYPKLESEFVYGDYKVYDVRK
MIAKSEQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFDSPTVAYSVLWA
KVEKGKSKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTIDRKRY
TSTKEVLDATLIHQSITGLYETRIDLSQLGGDSGGSGGSGGSTNLSDIIEKETGKQLVIQESI
LMLPEEVEEVIGNKPESDILVHTAYDESTDENVMLLTSDAPEYKPWALVIQDSNGENKIKMLS
GGSGGSGGSTNLSDIIEKETGKQLVIQESILMLPEEVEEVIGNKPESDILVHTAYDESTDENV
MLLTSDAPEYKPWALVIQDSNGENKIKMLSGGSKRTADGSEFEPKKKRKV
APOBEC3G(A3G)-BE4
(SEQ ID NO: 359)
MKRTADGSEFESPKKKRKVKPHFRNTVERMYRDTFSYNFYNRPILSRRNTVWLCYEVKTKGPS
RPPLDAKIFRGQVYSELKYHPEMRFFHWFSKWRKLHRDQEYEVTWYISWSPCTKCTRDMATFL
AEDPKVTLTIFVARLYYFWDPDYQEALRSLCQKRDGPRATMKIMNYDEFQHCWSKFVYSQREL
FEPWNNLPKYYILLHIMLGEILRHSMDPPTFTFNFNNEPWVRGRHETYLCYEVERMHNDTWVL
LNQRRGFLCNQAPHKHGFLEGRHAELCFLDVIPFWKLDLDQDYRVTCFTSWSPCFSCAQEMAK
FISKNKHVSLCIFTARIYDDQGRCQEGLRTLAEAGAKISIMTYSEFKHCWDTFVDHQGCPFQP
WDGLDEHSQDLSGRLRAILQNQENSGGSSGGSSGSETPGTSESATPESSGGSSGGSDKKYSIG
LAIGTNSVGWAVITDEYKVPSKKFKVEGNTDRHSIKKNEIGAEEFDSGETAEATREKRTARRR
YTRRKNRICYEQEIFSNEMAKVDDSFFHREEESFEVEEDKKHERHPIFGNIVDEVAYHEKYPT
IYHERKKEVDSTDKADEREIYEAEAHMIKFRGHFEIEGDENPDNSDVDKEFIQEVQTYNQEFE
ENPINASGVDAKAIESARESKSRREENEIAQEPGEKKNGEFGNEIAESEGETPNFKSNFDEAE
DAKEQESKDTYDDDEDNEEAQIGDQYADEFEAAKNESDAIEESDIERVNTEITKAPESASMIK
RYDEHHQDETEEKAEVRQQEPEKYKEIFFDQSKNGYAGYIDGGASQEEFYKFIKPIEEKMDGT
EEEEVKENREDEERKQRTFDNGSIPHQIHEGEEHAIERRQEDFYPFEKDNREKIEKIETFRIP
YYVGPEARGNSRFAWMTRKSEETITPWNFEEWDKGASAQSFIERMTNFDKNEPNEKVEPKHSE
EYEYFTVYNEETKVKYVTEGMRKPAFESGEQKKAIVDEEFKTNRKVTVKQEKEDYFKKIECFD
SVEISGVEDRFNASEGTYHDEEKIIKDKDFEDNEENEDIEEDIVETETEFEDREMIEEREKTY
AHEFDDKVMKQEKRRRYTGWGRESRKEINGIRDKQSGKTIEDFEKSDGFANRNFMQEIHDDSE
TFKEDIQKAQVSGQGDSEHEHIANEAGSPAIKKGIEQTVKWDEEVKVMGRHKPENIVIEMARE
NQTTQKGQKNSRERMKRIEEGIKEEGSQIEKEHPVENTQEQNEKEYEYYEQNGRDMYVDQEED
INRESDYDVDHIVPQSFEKDDSIDNKVETRSDKNRGKSDNVPSEEVVKKMKNYWRQEENAKEI
TQRKFDNETKAERGGESEEDKAGFIKRQEVETRQITKHVAQIEDSRMNTKYDENDKEIREVKV
ITEKSKEVSDFRKDFQFYKVREINNYHHAHDAYENAWGTALIKKYPKLESEFVYGDYKVYDVR
KMIAKSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYSVLV
VAKVEKGKSKKLKSVKELLGITIMERSSFEKNPIDFLEAKGYKEVKKDLIIKLPKYSLFELEN
GRKRMLASAGELQKGNELALPSKYVNFLYLASHYEKLKGSPEDNEQKQLFVEQHKHYLDEIIE
QISEFSKRVILADANLDKVLSAYNKHRDKPIREQAENIIHLFTLTNLGAPAAFKYFDTTIDRK
RYTSTKEVLDATLIHQSITGLYETRIDLSQLGGDSGGSGGSGGSTNLSDIIEKETGKQLVIQE
SILMLPEEVEEVIGNKPESDILVHTAYDESTDENVMLLTSDAPEYKPWALVIQDSNGENKIKM
LSGGSGGSGGSTNLSDIIEKETGKQLVIQESILMLPEEVEEVIGNKPESDILVHTAYDESTDE
NVMLLTSDAPEYKPWALVIQDSNGENKIKMLSGGSKRTADGSEFEPKKKRKV
AID-BE4
(SEQ ID NO: 360)
MKRTADGSEFESPKKKRKVDSLLMNRRKFLYQFKNVRWAKGRRETYLCYVVKRRDSATSFSLD
FGYLRNKNGCHVELLFLRYISDWDLDPGRCYRVTWFTSWSPCYDCARHVADFLRGNPNLSLRI
FTARLYFCEDRKAEPEGLRRLHRAGVQIAIMTFKDYFYCWNTFVENHERTFKAWEGLHENSVR
LSRQLRRILLPLYEVDDLRDAFRTLGLSGGSSGGSSGSETPGTSESATPESSGGSSGGSDKKY
SIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEK
YPTIYHLRKKLVDSTDKADLRLIYLALAHMIKFRGHFLIEGDLNPDNSDVDKLFIQLVQTYNQ
LFEENPINASGVDAKAILSARLSKSRRLENLIAQLPGEKKNGLFGNLIALSLGLTPNFKSNFD
LAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFLKDNREKIEKILTF
RIPYYVGPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLKEDYFKKI
ECFDSVEISGVEDRFNASLGTYHDLLKIIKDKDFLDNEENEDILEDIVLTLTLFEDREMIEER
LKTYAHLFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGRDMYV
DQELDINRLSDYDVDHIVPQSFLKDDSIDNKVLTRSDKNRGKSDNVPSEEWKKMKNYWRQLLN
AKLITQRKFDNLTKAERGGLSELDKAGFIKRQLVETRQITKHVAQILDSRMNTKYDENDKLIR
EVKVITLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWDPKKYGGFDSPTVA
YSVLWAKVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQLFVEQHKHYLD
EIIEQISEFSKRVILADANLDKVLSAYNKHRDKPIREQAENIIHLFTLTNLGAPAAFKYFDTT
IDRKRYTSTKEVEDATEIHQSITGEYETRIDESQLGGDSGGSGGSGGSTNLSDIIEKETGKQL
VIQESILMLPEEVEEVIGNKPESDILVHTAYDESTDENVMLLTSDAPEYKPWALVIQDSNGEN
KIKMLSGGSGGSGGSTNLSDIIEKETGKQLVIQESILMLPEEVEEVIGNKPESDILVHTAYDE
STDENVMLLTSDAPEYKPWALVIQDSNGENKIKMLSGGSKRTADGSEFEPKKKRKV
CDA-BE4
(SEQ ID NO: 361)
MKRTADGSEFESPKKKRKVTDAEYVRIHEKLDIYTFKKQFFNNKKSVSHRCYVLFELKRRGER
RACFWGYAVNKPQSGTERGIHAEIFSIRKVEEYLRDNPGQFTINWYSSWSPCADCAEKILEWY
NQELRGNGHTLKIWACKLYYEKNARNQIGLWNLRDNGVGLNVMVSEHYQCCRKIFIQSSHNQL
NENRWLEKTLKRAEKRRSELSIMIQVKILHTTKSPAVSGGSSGGSSGSEAPGASESAAPESSG
GSSGGSDKKYSIGLAIGTNSVGWAVITDEYKVPSKKFKVEGNTDRHSIKKNEIGAEEFDSGET
AEATREKRTARRRYTRRKNRICYEQEIFSNEMAKVDDSFFHREEESFEVEEDKKHERHPIFGN
IVDEVAYHEKYPTIYHERKKEVDSTDKADERElYEAEAHMIKFRGHFEIEGDENPDNSDVDKE
FIQEVQTYNQEFEENPINASGVDAKAIESARESKSRREENEIAQEPGEKKNGEFGNEIAESEG
ETPNFKSNFDEAEDAKEQESKDTYDDDEDNEEAQIGDQYADEFEAAKNESDAIEESDIERVNT
EITKAPESASMIKRYDEHHQDETEEKAEVRQQEPEKYKEIFFDQSKNGYAGYIDGGASQEEFY
KFIKPIEEKMDGTEEEEVKENREDEERKQRTFDNGSIPHQIHEGEEHAIERRQEDFYPFEKDN
REKIEKIETFRIPYYVGPEARGNSRFAWMTRKSEETITPWNFEEVVDKGASAQSFIERMTNFD
KNEPNEKVEPKHSEEYEYFTVYNEETKVKYVTEGMRKPAFESGEQKKAIVDEEFKTNRKVTVK
QEKEDYFKKIECFDSVEISGVEDRFNASEGTYHDEEKIIKDKDFEDNEENEDIEEDIVETETE
FEDREMIEEREKTYAHEFDDKVMKQEKRRRYTGWGRESRKEINGIRDKQSGKTIEDFEKSDGF
ANRNFMQEIHDDSETFKEDIQKAQVSGQGDSEHEHIANEAGSPAIKKGIEQTVKVVDEEVKVM
GRHKPENIVIEMARENQTTQKGQKNSRERMKRIEEGIKEEGSQIEKEHPVENTQEQNEKEYEY
YEQNGRDMYVDQEEDINRESDYDVDHIVPQSFEKDDSIDNKVETRSDKNRGKSDNVPSEEVVK
KMKNYWRQEENAKEITQRKFDNETKAERGGESEEDKAGFIKRQEVETRQITKHVAQIEDSRMN
TKYDENDKEIREVKVITEKSKEVSDFRKDFQFYKVREINNYHHAHDAYENAWGTAEIKKYPKE
ESEFVYGDYKVYDVRKMIAKSEQEIGKATAKYFFYSNIMNFFKTEITEANGEIRKRPEIETNG
ETGEIVWDKGRDFATVRKVESMPQVNIVKKTEVQTGGFSKESIEPKRNSDKEIARKKDWDPKK
YGGFDSPTVAYSVEVVAKVEKGKSKKEKSVKEEEGITIMERSSFEKNPIDFEEAKGYKEVKKD
EIIKEPKYSEFEEENGRKRMEASAGEEQKGNEEAEPSKYVNFEYEASHYEKEKGSPEDNEQKQ
EFVEQHKHYEDEIIEQISEFSKRVIEADANEDKVESAYNKHRDKPIREQAENIIHEFTETNEG
APAAFKYFDTTIDRKRYTSTKEVEDATEIHQSITGEYETRIDESQEGGDSGGSGGSGGSTNLS
DIIEKETGKQLVIQESILMLPEEVEEVIGNKPESDILVHTAYDESTDENVMLLTSDAPEYKPW
ALVIQDSNGENKIKMLSGGSGGSGGSTNLSDIIEKETGKQLVIQESILMLPEEVEEVIGNKPE
SDILVHTAYDESTDENVMLLTSDAPEYKPWALVIQDSNGENKIKMLSGGSKRTADGSEFEPKK
KRKV
FERNY-BE4
(SEQ ID NO: 362)
MKRTADGSEFESPKKKRKVFERNYDPRELRKETYLLYEIKWGKSGKLWRHWCQNNRTQHAEVY
FLENIFNARRFNPSTHCSITWYLSWSPCAECSQKIVDFLKEHPNVNLEIYVARLYYHEDERNR
QGLRDLVNSGVTIRIMDLPDYNYCWKTFVSDQGGDEDYWPGHFAPWIKQYSLKLSGGSSGGSS
GSETPGTSESATPESSGGSSGGSDKKYSIGLAIGTNSVGWAVITDEYKVPSKKFKVEGNTDRH
SIKKNEIGAEEFDSGETAEATRLKRTARRRYTRRKNRICYEQEIFSNEMAKVDDSFFHREEES
FEVEEDKKHERHPIFGNIVDEVAYHEKYPTIYHERKKEVDSTDKADEREIYEAEAHMIKFRGH
FEIEGDENPDNSDVDKEFIQEVQTYNQEFEENPINASGVDAKAIESARESKSRREENEIAQEP
GEKKNGEFGNEIAESEGETPNFKSNFDEAEDAKEQESKDTYDDDEDNEEAQIGDQYADEFEAA
KNESDAIEESDIERVNTEITKAPESASMIKRYDEHHQDETEEKAEVRQQEPEKYKEIFFDQSK
NGYAGYIDGGASQEEFYKFIKPIEEKMDGTEEEEVKENREDEERKQRTFDNGSIPHQIHEGEE
HAIERRQEDFYPFEKDNREKIEKIETFRIPYYVGPEARGNSRFAWMTRKSEETITPWNFEEVV
DKGASAQSFIERMTNFDKNEPNEKVEPKHSEEYEYFTVYNEETKVKYVTEGMRKPAFESGEQK
KAIVDEEFKTNRKVTVKQEKEDYFKKIECFDSVEISGVEDRFNASEGTYHDEEKIIKDKDFED
NEENEDIEEDIVETETEFEDREMIEEREKTYAHEFDDKVMKQEKRRRYTGWGRESRKEINGIR
DKQSGKTIEDFEKSDGFANRNFMQEIHDDSETFKEDIQKAQVSGQGDSEHEHIANEAGSPAIK
KGIEQTVKWDEEVKVMGRHKPENIVIEMARENQTTQKGQKNSRERMKRIEEGIKEEGSQIEKE
HPVENTQEQNEKEYEYYEQNGRDMYVDQEEDINRESDYDVDHIVPQSFEKDDSIDNKVETRSD
KNRGKSDNVPSEEWKKMKNYWRQEENAKEITQRKFDNETKAERGGESEEDKAGFIKRQEVETR
QITKHVAQIEDSRMNTKYDENDKEIREVKVITEKSKEVSDFRKDFQFYKVREINNYHHAHDAY
ENAVVGTAEIKKYPKEESEFVYGDYKVYDVRKMIAKSEQEIGKATAKYFFYSNIMNFFKTEIT
EANGEIRKRPEIETNGETGEIVWDKGRDFATVRKVESMPQVNIVKKTEVQTGGFSKESIEPKR
NSDKEIARKKDWDPKKYGGFDSPTVAYSVEWAKVEKGKSKKEKSVKEEEGITIMERSSFEKNP
IDFEEAKGYKEVKKDEIIKEPKYSEFEEENGRKRMEASAGEEQKGNEEAEPSKYVNFEYEASH
YEKEKGSPEDNEQKQEFVEQHKHYEDEIIEQISEFSKRVIEADANEDKVESAYNKHRDKPIRE
QAENIIHEFTETNEGAPAAFKYFDTTIDRKRYTSTKEVEDATEIHQSITGEYETRIDESQLGG
DSGGSGGSGGSTNLSDIIEKETGKQLVIQESILMLPEEVEEVIGNKPESDILVHTAYDESTDE
NVMLLTSDAPEYKPWALVIQDSNGENKIKMLSGGSGGSGGSTNLSDIIEKETGKQLVIQESIL
MLPEEVEEVIGNKPESDILVHTAYDESTDENVMLLTSDAPEYKPWALVIQDSNGENKIKMLSG
GSKRTADGSEFEPKKKRKV
Evolved APOBEC3A (eA3A)-BE4
(SEQ ID NO: 363)
MKRTADGSEFESPKKKRKVEASPASGPRHLMDPHIFTSNFNNGIGRHKTYLCYEVERLDNGTS
VKMDQHRGFLHGQAKNLLCGFYGRHAELRFLDLVPSLQLDPAQIYRVTWFISWSPCFSWGCAG
EVRAFLQENTHVRLRIFAARIYDYDPLYKEALQMLRDAGAQVSIMTYDEFKHCWDTFVDHQGC
PFQPWDGLDEHSQALSGRLRAILQNQGNSGGSSGGSSGSETPGTSESATPESSGGSSGGSDKK
YSIGLAIGTNSVGWAVITDEYKVPSKKFKVLGNTDRHSIKKNLIGALLFDSGETAEATRLKRT
ARRRYTRRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSDVDKLFIQLVQTYN
QLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPFLKDNREKIEKILT
FRIPYYVGPLARGNSRFAWMTRKSEETITPWNFEEVVDKGASAQSFIERMTNFDKNLPNEKVL
PKHSLLYEYFTVYNELTKVKYVTEGMRKPAFLSGEQKKAIVDLLFKTNRKVTVKQLKEDYFKK
IECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFMQLI
HDDSLTFKEDIQKAQVSGQGDSLHEHIANLAGSPAIKKGILQTVKVVDELVKVMGRHKPENIV
IEMARENQTTQKGQKNSRERMKRIEEGIKELGSQILKEHPVENTQLQNEKLYLYYLQNGRDMY
VDQELDINRLSDYDVDHIVPQSFLKDDSIDNKVLTRSDKNRGKSDNVPSEEWKKMKNYWRQLL
NAKLITQRKFDNLTKAERGGLSELDKAGFIKRQLVETRQITKHVAQILDSRMNTKYDENDKLI
REVKVITLKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKYGGFDSPTV
AYSVLWAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFVEQHKHYL
DEIIEQISEFSKRVILADANLDKVLSAYNKHRDKPIREQAENIIHLFTLTNLGAPAAFKYFDT
TIDRKRYTSTKEVEDATEIHQSITGEYETRIDESQEGGDSGGSGGSGGSTNLSDIIEKETGKQ
LVIQESILMLPEEVEEVIGNKPESDILVHTAYDESTDENVMLLTSDAPEYKPWALVIQDSNGE
NKIKMLSGGSGGSGGSTNLSDIIEKETGKQLVIQESILMLPEEVEEVIGNKPESDILVHTAYD
ESTDENVMLLTSDAPEYKPWALVIQDSNGENKIKMLSGGSKRTADGSEFEPKKKRKV
AALN-BE4
(SEQ ID NO: 364)
MKRTADGSEFESPKKKRKVSSETGPVAVDPTLRRRIEPHEFEVFFDPRELAAETCLLYEINWG
GRHSIWRHTSQNTNKHVEVNFIEKFTTERYFCPNTRCSITWFLSWSPCGECSRAITEFLSRYP
HVTLFIYIARLYHLANPRNRQGLRDLISSGVTIQIMTEQESGYCWRNFVNYSPSNEAHWPRYP
HLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVLGN
TDRHSIKKNLIGALLFDSGETAEATRLKRTARRRYTRRKNRICYEQEIFSNEMAKVDDSFFHR
EEESFEVEEDKKHERHPIFGNIVDEVAYHEKYPTIYHERKKEVDSTDKADEREIYEAEAHMIK
FRGHFEIEGDENPDNSDVDKEFIQEVQTYNQEFEENPINASGVDAKAILSARLSKSRRLENLI
AQLPGEKKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKYKEIFF
DQSKNGYAGYIDGGASQEEFYKFIKPILEKMDGTEEEEVKENREDEERKQRTFDNGSIPHQIH
EGEEHAIERRQEDFYPFEKDNREKIEKIETFRIPYYVGPLARGNSRFAWMTRKSEETITPWNF
EEVVDKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASEGTYHDEEKIIKDK
DFEDNEENEDIEEDIVETETEFEDREMIEEREKTYAHEFDDKVMKQEKRRRYTGWGRESRKEI
NGIRDKQSGKTIEDFEKSDGFANRNFMQLIHDDSLTFKEDIQKAQVSGQGDSLHEHIANLAGS
PAIKKGILQTVKVVDELVKVMGRHKPENIVIEMARENQTTQKGQKNSRERMKRIEEGIKELGS
QILKEHPVENTQLQNEKLYLYYLQNGRDMYVDQELDINRLSDYDVDHIVPQSFLKDDSIDNKV
LTRSDKNRGKSDNVPSEEWKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREINNYHH
AHDAYLNAWGTALIKKYPKLESEFVYGDYKVYDVRKMIAKSEQEIGKATAKYFFYSNIMNFFK
TEITLANGEIRKRPEIETNGETGEIVWDKGRDFATVRKVESMPQVNIVKKTEVQTGGFSKESI
EPKRNSDKEIARKKDWDPKKYGGFDSPTVAYSVEWAKVEKGKSKKEKSVKEEEGITIMERSSF
EKNPIDFEEAKGYKEVKKDEIIKEPKYSEFEEENGRKRMEASAGEEQKGNEEAEPSKYVNFEY
EASHYEKEKGSPEDNEQKQEFVEQHKHYEDEIIEQISEFSKRVIEADANEDKVESAYNKHRDK
PIREQAENIIHLFTLTNLGAPAAFKYFDTTIDRKRYTSTKEVLDATLIHQSITGLYETRIDLS
QLGGDSGGSGGSGGSTNLSDIIEKETGKQLVIQESILMLPEEVEEVIGNKPESDILVHTAYDE
STDENVMLLTSDAPEYKPWALVIQDSNGENKIKMLSGGSGGSGGSTNLSDIIEKETGKQLVIQ
ESILMLPEEVEEVIGNKPESDILVHTAYDESTDENVMLLTSDAPEYKPWALVIQDSNGENKIK
MLSGGSKRTADGSEFEPKKKRKV
BE4max, modified with SpCas9-NG (“BE4-NG”)
(SEQ ID NO: 365)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHWPRYP
HLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVLGN
TDRHSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKEVDSTDKADEREIYEAEAHMIK
FRGHFEIEGDENPDNSDVDKEFIQEVQTYNQEFEENPINASGVDAKAIESARESKSRREENEI
AQEPGEKKNGEFGNEIAESEGETPNFKSNFDEAEDAKEQESKDTYDDDEDNEEAQIGDQYADE
FEAAKNESDAIEESDIERVNTEITKAPESASMIKRYDEHHQDETEEKAEVRQQEPEKYKEIFF
DQSKNGYAGYIDGGASQEEFYKFIKPIEEKMDGTEEEEVKENREDEERKQRTFDNGSIPHQIH
EGEEHAIERRQEDFYPFEKDNREKIEKIETFRIPYYVGPEARGNSRFAWMTRKSEETITPWNF
EEVVDKGASAQSFIERMTNFDKNEPNEKVEPKHSEEYEYFTVYNEETKVKYVTEGMRKPAFES
GEQKKAIVDEEFKTNRKVTVKQEKEDYFKKIECFDSVEISGVEDRFNASEGTYHDEEKIIKDK
DFEDNEENEDIEEDIVETETEFEDREMIEEREKTYAHEFDDKVMKQEKRRRYTGWGRESRKEI
NGIRDKQSGKTIEDFEKSDGFANRNFMQEIHDDSETFKEDIQKAQVSGQGDSEHEHIANEAGS
PAIKKGIEQTVKVVDEEVKVMGRHKPENIVIEMARENQTTQKGQKNSRERMKRIEEGIKEEGS
QIEKEHPVENTQEQNEKEYEYYEQNGRDMYVDQEEDINRESDYDVDHIVPQSFEKDDSIDNKV
ETRSDKNRGKSDNVPSEEWKKMKNYWRQEENAKEITQRKFDNETKAERGGESEEDKAGFIKRQ
EVETRQITKHVAQILDSRMNTKYDENDKLIREVKVITLKSKLVSDFRKDFQFYKVREINNYHH
AHDAYLNAVVGTAEIKKYPKEESEFVYGDYKVYDVRKMIAKSEQEIGKATAKYFFYSNIMNFF
KTEITEANGEIRKRPEIETNGETGEIVWDKGRDFATVRKVESMPQVNIVKKTEVQTGGFSKES
IRPKRNSDKEIARKKDWDPKKYGGFVSPTVAYSVEWAKVEKGKSKKEKSVKEEEGITIMERSS
FEKNPIDFEEAKGYKEVKKDEIIKEPKYSEFEEENGRKRMEASARFEQKGNEEAEPSKYVNFE
YEASHYEKEKGSPEDNEQKQEFVEQHKHYEDEIIEQISEFSKRVIEADANEDKVESAYNKHRD
KPIREQAENIIHEFTETNEGAPRAFKYFDTTIDRKVYRSTKEVEDATEIHQSITGEYETRIDL
SQLGGDSGGSGGSGGSTNLSDIIEKETGKQLVIQESILMLPEEVEEVIGNKPESDILVHTAYD
ESTDENVMLLTSDAPEYKPWALVIQDSNGENKIKMLSGGSGGSGGSTNLSDIIEKETGKQLVI
QESILMLPEEVEEVIGNKPESDILVHTAYDESTDENVMLLTSDAPEYKPWALVIQDSNGENKI
KMLSGGSKRTADGSEFEPKKKRKV
rAPOBEC1-XTEN-SpCas9 nickase-UGI-NLS
(BE3, SEQ ID NO: 366)
MSSETGPVAVDPTLRRRIEPHEFEVFFDPRELRKETCLLYEINWGGRHSIWRHTSQNTNKHVE
VNFIEKFTTERYFCPNTRCSITWFLSWSPCGECSRAITEFLSRYPHVTLFIYIARLYHHADPR
NRQGLRDLISSGVTIQIMTEQESGYCWRNFVNYSPSNEAHWPRYPHLWVRLYVLELYCllLGL
PPCLNILRRKQPQLTFFTIALQSCHYQRLPPHILWATGLKSGSETPGTSESATPESDKKYSIG
LAIGTNSVGWAVITDEYKVPSKKFKVLGNTDRHSIKKNLIGALLFDSGETAEATRLKRTARRR
YTRRKNRICYLQEIFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAE
DAKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECF
DSVEISGVEDRFNASLGTYHDLLKllKDKDFLDNEENEDILEDIVLTlTLFEDREMIEERLKT
YAHLFDDKVMKQLKRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRDMYVDQE
LDINRLSDYDVDHIVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMNTKYDENDKLIREV
KVITLKSKLVSDFRKDFQFYKVREINNYHHAHDAYLNAVVGTALIKKYPKLESEFVYGDYKVY
DVRKMIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKKDLllKLPKYSLFE
LENGRKRMLASAGELQKGNELALPSKYVNFLYLASHYEKLKGSPEDNEQKQLFVEQHKHYLDE
llEQISEFSKRVILADANLDKVLSAYNKHRDKPIREQAENllHLFTLTNLGAPAAFKYFDTTI
DRKRYTSTKEVLDATLIHQSITGLYETRIDLSQLGGDSGGSTNLSDllEKETGKQLVIQESIL
MLPEEVEEVIGNKPESDILVHTAYDESTDENVMLLTSDAPEYKPWALVIQDSNGENKIKMLSG
GSPKKKRKV
rAPOBEC1-XTEN-SaCas9n-UGI-NLS) (SaBE3 and
SaBE3.9max)
(SEQ ID NO: 367)
MSSETGPVAVDPTLRRRIEPHEFEVFFDPRELRKETCLLYEINWGGRHSIWRHTSQNTNKHVE
VNFIEKFTTERYFCPNTRCSITWFLSWSPCGECSRAITEFLSRYPHVTLFIYIARLYHHADPR
NRQGLRDLISSGVTIQIMTEQESGYCWRNFVNYSPSNEAHWPRYPHLWVRLYVLELYCIILGL
PPCLNILRRKQPQLTFFTIALQSCHYQRLPPHILWATGLKSGSETPGTSESATPESKRNYILG
LDIGITSVGYGIIDYETRDVIDAGVRLFKEANVENNEGRRSKRGARRLKRRRRHRIQRVKKLL
FDYNLLTDHSELSGINPYEARVKGLSQKLSEEEFSAALLHLAKRRGVHNVNEVEEDTGNELST
KEQISRNSKALEEKYVAELQLERLKKDGEVRGSINRFKTSDYVKEAKQLLKVQKAYHQLDQSF
IDTYIDLLETRRTYYEGPGEGSPFGWKDIKEWYEMLMGHCTYFPEELRSVKYAYNADLYNALN
DLNNLVITRDENEKLEYYEKFQIIENVFKQKKKPTLKQIAKEILVNEEDIKGYRVTSTGKPEF
TNLKVYHDIKDITARKEIIENAELLDQIAKILTIYQSSEDIQEELTNLNSELTQEEIEQISNL
KGYTGTHNLSLKAINLILDELWHTNDNQIAIFNRLKLVPKKVDLSQQKEIPTTLVDDFILSPV
VKRSFIQSIKVINAIIKKYGLPNDIIIELAREKNSKDAQKMINEMQKRNRQTNERIEEIIRTT
GKENAKYLIEKIKLHDMQEGKCLYSLEAIPLEDLLNNPFNYEVDHIIPRSVSFDNSFNNKVLV
KQEEASKKGNRTPFQYLSSSDSKISYETFKKHILNLAKGKGRISKTKKEYLLEERDINRFSVQ
KDFINRNLVDTRYATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYKEIFITPHQIKHIK
DFKDYKYSHRVDKKPNRELINDTLYSTRKDDKGNTLIVNNLNGLYDKDNDKLKKLINKSPEKL
LMYHHDPQTYQKLKLIMEQYGDEKNPLYKYYEETGNYLTKYSKKDNGPVIKKIKYYGNKLNAH
LDITDDYPNSRNKVVKLSLKPYRFDVYLDNGVYKFVTVKNLDVIKKENYYEVNSKCYEEAKKL
KKISNQAEFIASFYNNDLIKINGELYRVIGVNNDLLNRIEVNMIDITYREYLENMNDKRPPRI
IKTIASKTQSIKKYSTDILGNLYEVKSKKHPQIIKKGSGGSTNLSDIIEKETGKQLVIQESIL
MLPEEVEEVIGNKPESDILVHTAYDESTDENVMLLTSDAPEYKPWALVIQDSNGENKIKMLSG
GSPKKKRKV
AID-VRQR-BE4max
(SEQ ID NO: 368)
MDSLLMNRRKFLYQFKNVRWAKGRRETYLCYVVKRRDSATSFSLDFGYLRNKNGCHVELLFLR
YISDWDLDPGRCYRVTWFTSWSPCYDCARHVADFLRGNPNLSLRIFTARLYFCEDRKAEPEGL
RRLHRAGVQIAIMTFKDYFYCWNTFVENHERTFKAWEGLHENSVRLSRQLRRILLPLYEVDDL
RDAFRTLGLSGGSSGGSSGSETPGTSESATPESSGGSSGGSDKKYSIGLAIGTNSVGWAVITD
EYKVPSKKFKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYAHLFDDKVMKQLKR
RRYTGWGRLSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDVDHIV
PQSFLKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDFRK
DFQFYKVREINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSMPQVNI
VKKTEVQTGGFSKESILPKRNSDKLIARKKDWDPKKYGGFVSPTVAYSVLVVAKVEKGKSKKL
KSVKELLGITIMERSSFEKNPIDFLEAKGYKEVKKDLIIKLPKYSLFELENGRKRMLASAREL
QKGNELALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKQYRSTKEVLDAT
LIHQSITGLYETRIDLSQLGGDSGGSGGSGGSTNLSDIIEKETGKQLVIQESILMLPEEVEEV
IGNKPESDILVHTAYDESTDENVMLLTSDAPEYKPWALVIQDSNGENKIKMLSGGSGGSGGST
NLSDIIEKETGKQLVIQESILMLPEEVEEVIGNKPESDILVHTAYDESTDENVMLLTSDAPEY
KPWALVIQDSNGENKIKMLSGGSKRTADGSEFEPKKKRKV
BE4max-SaKKH
(SEQ ID NO: 369)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHWPRYP
HLWVRLYVLELYCIILGLPPCLNILRRKQPQLTFFTIALQSCHYQRLPPHILWATGLKSGGSS
GGSSGSETPGTSESATPESSGGSSGGSGKRNYILGLAIGITSVGYGIIDYETRDVIDAGVRLF
KEANVENNEGRRSKRGARRLKRRRRHRIQRVKKLLFDYNLETDHSEESGINPYEARVKGESQK
ESEEEFSAAEEHEAKRRGVHNVNEVEEDTGNEESTKEQISRNSKAEEEKYVAEEQEEREKKDG
EVRGSINRFKTSDYVKEAKQEEKVQKAYHQEDQSFIDTYIDEEETRRTYYEGPGEGSPFGWKD
IKEWYEMEMGHCTYFPEEERSVKYAYNADEYNAENDENNEVITRDENEKEEYYEKFQIIENVF
KQKKKPTEKQIAKEIEVNEEDIKGYRVTSTGKPEFTNEKVYHDIKDITARKEIIENAEEEDQI
AKIETIYQSSEDIQEEETNENSEETQEEIEQISNEKGYTGTHNESEKAINEIEDEEWHTNDNQ
IAIFNREKEVPKKVDESQQKEIPTTEVDDFIESPVVKRSFIQSIKVINAIIKKYGEPNDIIIE
EAREKNSKDAQKMINEMQKRNRQTNERIEEIIRTTGKENAKYEIEKIKEHDMQEGKCEYSEEA
IPEEDEENNPFNYEVDHIIPRSVSFDNSFNNKVEVKQEENSKKGNRTPFQYESSSDSKISYET
FKKHIENEAKGKGRISKTKKEYEEEERDINRFSVQKDFINRNEVDTRYATRGEMNEERSYFRV
NNEDVKVKSINGGFTSFERRKWKFKKERNKGYKHHAEDAEIIANADFIFKEWKKEDKAKKVME
NQMFEEKQAESMPEIETEQEYKEIFITPHQIKHIKDFKDYKYSHRVDKKPNRKEINDTEYSTR
KDDKGNTEIVNNENGEYDKDNDKEKKEINKSPEKEEMYHHDPQTYQKEKEIMEQYGDEKNPEY
KYYEETGNYETKYSKKDNGPVIKKIKYYGNKENAHEDITDDYPNSRNKVVKESEKPYRFDVYE
DNGVYKFVTVKNEDVIKKENYYEVNSKCYEEAKKLKKISNQAEFIASFYKNDLIKINGELYRV
IGVNNDLLNRIEVNMIDITYREYEENMNDKRPPHIIKTIASKTQSIKKYSTDIEGNEYEVKSK
KHPQIIKKGSGGSGGSGGSTNESDIIEKETGKQEVIQESIEMEPEEVEEVIGNKPESDIEVHT
AYDESTDENVMEETSDAPEYKPWAEVIQDSNGENKIKMESGGSGGSGGSTNESDIIEKETGKQ
EVIQESIEMEPEEVEEVIGNKPESDIEVHTAYDESTDENVMEETSDAPEYKPWAEVIQDSNGE
NKIKMESGGSKRTADGSEFEPKKKRKV
BE4max-NRRH
(SEQ ID NO: 370)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHWPRYP
HLWVRLYVLELYCIILGLPPCLNILRRKQPQLTFFTIALQSCHYQRLPPHILWATGLKSGGSS
GGSSGSETPGTSESATPESSGGSSGGSDKKYSIGLTIGTNSVGWAVITDEYKVPSKKFKVEGN
TDRHSIKKNEIGAEEFDSGETAEATRLKRTARRRYTRRKNRICYEQEIFSNEMAKVDDSFFHR
EEESFEVEEDKKHERHPIFGNIVDEVAYHEKYPTIYHERKKEVDSTDKADEREIYEAEAHMIK
FRGHFEIEGDENPDNSDVDKEFIQEVQTYNQEFEENPINASGVDAKAIESARESKSRREENEI
AQEPGEKKNGEFGNEIAESEGETPNFKSNFDEAEDAKEQESKDTYDDDEDNEEAQIGDQYADE
FEAAKNESDAIEESDIERVNTEITKAPESASMVKRYDEHHQDETEEKAEVRQQEPEKYKEIFF
DQSKNGYAGYIDGGASQEEFYKFIKPIEEKMDGTEEEEVKENREDEERKQRTFDNGIIPHQIH
EGEEHAIERRQGDFYPFEKDNREKIEKIETFRIPYYVGPEARGNSRFAWMTRKSEETITPWNF
EEVVDKGASAQSFIERMTNFDKNEPNEKVEPKHSEEYEYFTVYNEETKVKYVTEGMRKPAFES
GEQKKAIVDEEFKTNRKVTVKQEKEDYFKKIECFDSVEISGVEDRFNASEGTYHDEEKIIKDK
DFEDNEENEDIEEDIVETETEFEDREMIEEREKTYAHEFDDKVMKQEKRERYTGWGRESRKEI
NGIRDKQSGKTIEDFEKSDGFANRNFMQEIHDDSETFKEDIQKAQVSGQGDSEHEHIANEAGS
PAIKKGIEQTVKWDEEVKVMGGHKPENIVIEMARENQTTQKGQKNSRERMKRIEEGIKEEGSQ
IEKEHPVENTQEQNEKEYEYYEQNGRDMYVDQEEDINRESDYDVDHIVPQSFEKDDSIDNKVE
TRSDKNRGKSDNVPSEEVVKKMKNYWRQEENAKEITQRKFDNETKAERGGESEEDKAGFIKRQ
EVETRQITKHVAQIEDSRMNTKYDENDKEIREVKVITEKSKEVSDFRKDFQFYKVREINNYHH
AHDAYENAWGTAEIKKYPKEESEFVYGDYKVYDVRKMIAKSEQEIGKATAKYFFYSNIMNFFK
TEITEANGEIRKRPEIETNGETGEIVWDKGRDFATVRKVESMPQVNIVKKTEVQTGGFSKESI
EPKGNSDKEIARKKDWDPKKYGGFNSPTAAYSVEWAKVEKGKSKKEKSVKEEEGITIMERSSF
EKNPIGFEEAKGYKEVKKDEIIKEPKYSEFEEENGRKRMEASAGVEHKGNEEAEPSKYVNFEY
EASHYEKEKGSPEDNEQKQEFVEQHKHYEDEIIEQISEFSKRVIEADANEDKVESAYNKHRDK
PIREQAENIIHEFTETNEGVPAAFKYFDTTIDKKRYTSTKEVEDATEIHQSITGEYETRIDLS
QLGGDSGGSGGSGGSTNLSDIIEKETGKQLVIQESILMLPEEVEEVIGNKPESDILVHTAYDE
STDENVMLLTSDAPEYKPWALVIQDSNGENKIKMLSGGSGGSGGSTNLSDIIEKETGKQLVIQ
ESILMLPEEVEEVIGNKPESDILVHTAYDESTDENVMLLTSDAPEYKPWALVIQDSNGENKIK
MLSGGSKRTADGSEFEPKKKRKV
BE4max-VQR
(SEQ ID NO: 371)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHWPRYP
HLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVLGN
TDRHSIKKNLIGALLFDSGETAEATREKRTARRRYTRRKNRICYEQEIFSNEMAKVDDSFFHR
EEESFEVEEDKKHERHPIFGNIVDEVAYHEKYPTIYHERKKEVDSTDKADEREIYEAEAHMIK
FRGHFEIEGDENPDNSDVDKEFIQEVQTYNQEFEENPINASGVDAKAIESARESKSRREENEI
AQEPGEKKNGEFGNEIAESEGETPNFKSNFDEAEDAKEQESKDTYDDDEDNEEAQIGDQYADE
FEAAKNESDAIEESDIERVNTEITKAPESASMIKRYDEHHQDETEEKAEVRQQEPEKYKEIFF
DQSKNGYAGYIDGGASQEEFYKFIKPIEEKMDGTEEEEVKENREDEERKQRTFDNGSIPHQIH
EGEEHAIERRQEDFYPFEKDNREKIEKIETFRIPYYVGPEARGNSRFAWMTRKSEETITPWNF
EEVVDKGASAQSFIERMTNFDKNEPNEKVEPKHSEEYEYFTVYNEETKVKYVTEGMRKPAFES
GEQKKAIVDEEFKTNRKVTVKQEKEDYFKKIECFDSVEISGVEDRFNASEGTYHDEEKIIKDK
DFEDNEENEDIEEDIVETETEFEDREMIEEREKTYAHEFDDKVMKQEKRRRYTGWGRESRKEI
NGIRDKQSGKTIEDFEKSDGFANRNFMQEIHDDSETFKEDIQKAQVSGQGDSEHEHIANEAGS
PAIKKGIEQTVKWDEEVKVMGRHKPENIVIEMARENQTTQKGQKNSRERMKRIEEGIKEEGSQ
IEKEHPVENTQEQNEKEYEYYEQNGRDMYVDQEEDINRESDYDVDHIVPQSFEKDDSIDNKVE
TRSDKNRGKSDNVPSEEWKKMKNYWRQEENAKEITQRKFDNETKAERGGESEEDKAGFIKRQE
VETRQITKHVAQIEDSRMNTKYDENDKEIREVKVITEKSKEVSDFRKDFQFYKVREINNYHHA
HDAYENAWGTAEIKKYPKEESEFVYGDYKVYDVRKMIAKSEQEIGKATAKYFFYSNIMNFFKT
EITEANGEIRKRPEIETNGETGEIVWDKGRDFATVRKVESMPQVNIVKKTEVQTGGFSKESIE
PKRNSDKEIARKKDWDPKKYGGFVSPTVAYSVEWAKVEKGKSKKEKSVKEEEGITIMERSSFE
KNPIDFEEAKGYKEVKKDEIIKEPKYSEFEEENGRKRMEASAGEEQKGNEEAEPSKYVNFEYE
ASHYEKEKGSPEDNEQKQEFVEQHKHYEDEIIEQISEFSKRVIEADANEDKVESAYNKHRDKP
IREQAENIIHEFTETNEGAPAAFKYFDTTIDRKQYRSTKEVEDATEIHQSITGLYETRIDLSQ
LGGDSGGSGGSGGSTNLSDIIEKETGKQLVIQESILMLPEEVEEVIGNKPESDILVHTAYDES
TDENVMLLTSDAPEYKPWALVIQDSNGENKIKMLSGGSGGSGGSTNLSDIIEKETGKQLVIQE
SILMLPEEVEEVIGNKPESDILVHTAYDESTDENVMLLTSDAPEYKPWALVIQDSNGENKIKM
LSGGSKRTADGSEFEPKKKRKV
BE4max-VRQR
(SEQ ID NO: 372)
MKRTADGSEFESPKKKRKVSSETGPVAVDPTLRRRIEPHEFEVFFDPRELRKETCLLYEINWG
GRHSIWRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAHWPRYP
HLWVRLYVLELYCIILGLPPCLNILRRKQPQLTFFTIALQSCHYQRLPPHILWATGLKSGGSS
GGSSGSETPGTSESATPESSGGSSGGSDKKYSIGLAIGTNSVGWAVITDEYKVPSKKFKVLGN
TDRHSIKKNLIGALLFDSGETAEATREKRTARRRYTRRKNRICYEQEIFSNEMAKVDDSFFHR
EEESFEVEEDKKHERHPIFGNIVDEVAYHEKYPTIYHERKKEVDSTDKADEREIYEAEAHMIK
FRGHFEIEGDENPDNSDVDKEFIQEVQTYNQEFEENPINASGVDAKAIESARESKSRREENEI
AQEPGEKKNGEFGNEIAESEGETPNFKSNFDEAEDAKEQESKDTYDDDEDNEEAQIGDQYADE
FEAAKNESDAIEESDIERVNTEITKAPESASMIKRYDEHHQDETEEKAEVRQQEPEKYKEIFF
DQSKNGYAGYIDGGASQEEFYKFIKPIEEKMDGTEEEEVKENREDEERKQRTFDNGSIPHQIH
EGEEHAIERRQEDFYPFEKDNREKIEKIETFRIPYYVGPEARGNSRFAWMTRKSEETITPWNF
EEVVDKGASAQSFIERMTNFDKNEPNEKVEPKHSEEYEYFTVYNEETKVKYVTEGMRKPAFES
GEQKKAIVDEEFKTNRKVTVKQEKEDYFKKIECFDSVEISGVEDRFNASEGTYHDEEKIIKDK
DFEDNEENEDIEEDIVETETEFEDREMIEEREKTYAHEFDDKVMKQEKRRRYTGWGRESRKEI
NGIRDKQSGKTIEDFEKSDGFANRNFMQEIHDDSETFKEDIQKAQVSGQGDSEHEHIANEAGS
PAIKKGIEQTVKWDEEVKVMGRHKPENIVIEMARENQTTQKGQKNSRERMKRIEEGIKEEGSQ
IEKEHPVENTQEQNEKEYEYYEQNGRDMYVDQEEDINRESDYDVDHIVPQSFEKDDSIDNKVE
TRSDKNRGKSDNVPSEEWKKMKNYWRQEENAKEITQRKFDNETKAERGGESEEDKAGFIKRQE
VETRQITKHVAQIEDSRMNTKYDENDKEIREVKVITEKSKEVSDFRKDFQFYKVREINNYHHA
HDAYENAWGTAEIKKYPKEESEFVYGDYKVYDVRKMIAKSEQEIGKATAKYFFYSNIMNFFKT
EITEANGEIRKRPEIETNGETGEIVWDKGRDFATVRKVESMPQVNIVKKTEVQTGGFSKESIE
PKRNSDKEIARKKDWDPKKYGGFVSPTVAYSVEWAKVEKGKSKKEKSVKEEEGITIMERSSFE
KNPIDFEEAKGYKEVKKDEIIKEPKYSEFEEENGRKRMEASAREEQKGNEEAEPSKYVNFEYE
ASHYEKEKGSPEDNEQKQEFVEQHKHYEDEIIEQISEFSKRVIEADANEDKVESAYNKHRDKP
IREQAENIIHEFTETNEGAPAAFKYFDTTIDRKQYRSTKEVEDATEIHQSITGLYETRIDLSQ
LGGDSGGSGGSGGSTNLSDIIEKETGKQLVIQESILMLPEEVEEVIGNKPESDILVHTAYDES
TDENVMLLTSDAPEYKPWALVIQDSNGENKIKMLSGGSGGSGGSTNLSDIIEKETGKQLVIQE
SILMLPEEVEEVIGNKPESDILVHTAYDESTDENVMLLTSDAPEYKPWALVIQDSNGENKIKM
LSGGSKRTADGSEFEPKKKRKV

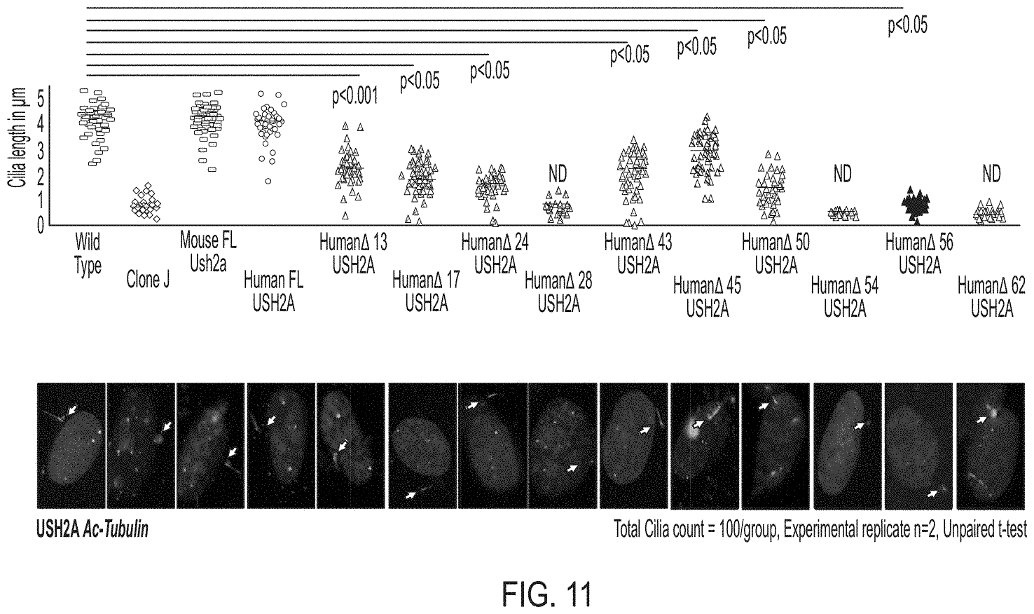

These disclosed CBEs exhibit low off-target editing frequencies, and in particular low Cas9-independent off-target editing frequencies, while exhibiting high on-target editing efficiencies. For example, the YE1-BE4, YE1-CP1028, YE1-SpCas9-NG, R33A-BE4, and R33A+K34A-BE4-CP1028 base editors may exhibit off-target editing frequencies of less than 0.75% (e.g., about 0.4% or less) while maintaining on-target editing efficiencies of about 60% or more, in target sequences in mammalian cells. (See, e.g., FIGS. 11, 15A, 15B and 17.) The Examples of the present disclosure suggest that CBEs with cytosine deaminases that have a low instrinsic catalytic efficiency (k_{cat}/Kₘ) for cytosine-containing ssDNA substrates exhibit reduced Cas9-independent off-target deamination.

In some embodiments, the base editor comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 350-372, or to any of the base editors described herein. In some embodiments, the base editor comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NOs: 350-372, or any of the base editors described herein. In some embodiments, the base editor comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1750, or at least 1800 identical contiguous amino acid residues as compared to any one of the amino acid sequences set forth in SEQ ID NOs: 350-372, or any of the base editors described herein. In some embodiments, the base editor (base editor) comprises the amino acid sequence of SEQ ID NO: 350, or a variant thereof that is at lest 60%, at least 65%, at least 70%, at least 75%, at least 80%, at least 85%, at least 90%, at least 95%, at least 96%, at least 97%, at least 98%, at least 99%, or at least 99.5% identical.

In some embodiments, the base editors described herein are capable of modifying a specific nucleotide base without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g. mutate or deaminate) a specific nucleotide within a nucleic acid, without generating a large number of insertions or deletions (i.e., indels) in the nucleic acid. In certain embodiments, any of the base editors described herein are capable of generating a greater proportion of intended modifications (e.g., point mutations or deaminations) versus indels. In some embodiments, the base editors described herein are capable of generating a ratio of intended point mutations to indels that is greater than 1:1. In some embodiments, the base editors described herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method. In some embodiments, to calculate indel frequencies, sequencing reads are scanned for exact matches to two 10-bp sequences that flank both sides of a window in which indels might occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively.

In some embodiments, the base editors described herein are capable of limiting formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors described herein are capable of limiting the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, an number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some aspects of the disclosure are based on the recognition that any of the base editors described herein are capable of efficiently generating an intended mutation, such as a point mutation, in a nucleic acid (e.g. a nucleic acid within a genome of a subject) without generating a significant number of unintended mutations, such as unintended point mutations. In some embodiments, an intended mutation is a mutation that is generated by a specific base editor bound to a gRNA, specifically designed to generate the intended mutation. In some embodiments, the intended mutation is a mutation associated with a disease or disorder. In some embodiments, the intended mutation is an adenine (A) to guanine (G) point mutation associated with a disease or disorder. In some embodiments, the intended mutation is a thymine (T) to cytosine (C) point mutation associated with a disease or disorder. In some embodiments, the intended mutation is an adenine (A) to guanine (G) point mutation within the coding region of a gene. In some embodiments, the intended mutation is a thymine (T) to cytosine (C) point mutation within the coding region of a gene. In some embodiments, the intended mutation is a point mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors described herein are capable of generating a ratio of intended mutations to unintended mutations (e.g., intended point mutations:unintended point mutations) that is greater than 1:1. In some embodiments, any of the base editors described herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least

### Reduced Off-Target Effects

Guide RNA-dependent off-target base editing has been reduced through strategies including installation of mutations that increase DNA specificity into the Cas9 domain of any of the disclosed base editors, adding 5′ guanosine nucleotides to the sgRNA, or delivery of the base editor as a ribonucleoprotein complex (RNP). Guide RNA-independent off-target editing can arise from binding of the deaminase domain of a base editor to C or A bases in a Cas9-independent manner. The off-target effects of the disclosed base editors may be measured using assays and methods disclosed in International Application No. PCT/US2020/624628, filed Nov. 25, 2020, and International Publication No. WO 2020/160514, published Aug. 6, 2020, each of which is incorporated herein by reference. It will be appreciated that various methods and assays exist in the art for evaluation of the off-target effects of any of the disclosed base editors, such as EndoV-Seq, GUIDE-Seq, CIRCLE-Seq, and Cas-OFFinder.

In some aspects, provided herein are base editors and methods of editing DNA by contacting DNA with any of these disclosed base editors that generate (or cause) reduced off-target effects. In various embodiments, methods are designed for determining the off-target editing frequencies of napDNAbp domain-independent (e.g., Cas9-independent) (or gRNA-independent) off-target editing events. Editing events may comprise deamination events of a BE. Off-target deamination events that are dependent on the napDNAbp-guide RNA complex tend to be in sequences that have high sequence identity (e.g., greater than 60% sequence identity) to the target sequence. These types of events arise because of imperfect hybridization of the napDNAbp-guide RNA complex to sequences that share identity with the target sequence. In contrast, off-target events that occur independently of the napDNAbp-guide RNA complex arise as a result of stochastic binding of rhe base editor to DNA sequences (often sequences that do not share high sequence identity with the target sequence) due to an intrinsic affinity of the base editor of the nucleotide modification domain (e.g., the deaminase domain) of the base editor with DNA. NapDNAbp-independent (e.g., Cas9-independent) editing events arise in particular when the base editor is overexpressed in the system under evaluation, such as a cell or a subject.

The disclosed BEs exhibit low off-target editing frequencies, and in particular low off-target editing frequencies in the USH2A genomic sequence or cDNA sequence, while exhibiting high on-target editing efficiencies. In some embodiments, the disclosed Bes exhibit low off-target editing frequencies, and in particular low Cas9-independent off-target editing frequencies, while exhibiting high on-target editing efficiencies when used a variety of Cas homologs and other napDNAbps.

In some embodiments, the disclosed base editors cause off-target DNA editing (e.g. off-target deamination) frequencies of less than 1.5% (such as less than 1.25%, less than 1.0%, less than 0.75%, or less than 0.5%). The disclosed base editors may further provide (or yield) on-target editing efficiencies of greater than 60% (such as greater than 70%, greater than 75%, greater than 80%, greater than 85%, greater than 89%, greater than 90%, greater than 95%, or greater than 98%) at the target nucleobase pair for one or more base editors under evaluation. These methods may yield an on-target editing efficiency of greater than 50% and a frequency of off-target editing of less than 1.5% for one or more base editors under consideration. In some embodiments, such as methods using the ABE8 editor, the disclosed methods may yield an on-target editing efficiency of 89.4% and a frequency of off-target editing of 1.77%. In some embodiments, such as methods using the BE3.9 editor, the disclosed methods may yield an on-target editing efficiency of 59.5% and a frequency of off-target editing of less than 5% or less than 1%.

In some embodiments, the disclosed BEs and editing methods comprising the step of contacting a DNA with any of the disclosed BEs result in an actual or average off-target DNA editing frequency of about 2.0% or less, 1.75% or less, 1.5% or less, 1.2% or less, 1% or less, 0.9% or less, 0.8% or less, 0.75% or less, 0.7% or less, 0.65% or less, or 0.6% or less. In some embodiments, the disclosed editing methods result in an actual or average off-target DNA editing frequency of 0.5%, less than 0.5%, less than 0.4%, less than 0.35%, less than 0.3%, less than 0.25%, less than 0.2%, or less than 0.1%. In a particular embodiment, the methods result in an actual or average off-target DNA editing frequency of about 0.32% to about 1.3% (for instance, methods for evaluating the off-target frequencies of ABEs). These off-target editing frequencies may be obtained in sequences having any level of sequence identity to the target sequence. As used herein to refer to off-target DNA editing frequencies, the modifier “average” refers to a mean value over all editing events detected at sites other than a given target nucleobase pair (e.g., as detected by high-throughput sequencing such as next-generation sequencing (NGS), or Sanger sequencing).

In some embodiments, the disclosed editing methods further result in an actual or average Cas9-independent off-target DNA editing frequency of about 2.0% or less, 1.75% or less, 1.5% or less, 1.2% or less, 1% or less, 0.9% or less, 0.8% or less, 0.75% or less, 0.7% or less, 0.65% or less, or 0.6% or less. In other words, the disclosed editing methods further result in an actual or average off-target DNA editing frequency of about 2.0% or less, 1.75% or less, 1.5% or less, 1.2% or less, 1% or less, 0.9% or less, 0.8% or less, 0.75% or less, 0.7% or less, 0.65% or less, or 0.6% or less in sequences having 60% or less sequence identity to the target sequence. In some embodiments, the disclosed editing methods result in an actual or average off-target DNA editing frequency 0.5%, less than 0.5%, less than 0.4%, less than 0.35%, less than 0.3%, less than 0.25%, less than 0.2%, or less than 0.1%, in sequences having 60% or less sequence identity to the target sequence. In some embodiments, these editing frequencies are obtained in sequences comprising protospacer sequences having 5, 6, 7, 8, 9, 10, or more than 10 mismatches relative to protospacer sequence of the target sequence. In a particular embodiment, the methods result in an actual or average Cas9-independent off-target DNA editing frequency of 0.4% or less.

In various embodiments, the disclosed editing methods result in an on-target DNA base editing efficiency of at least about 35%, 40%, 50%, 60%, 70%, 80%, 85%, 90%, 95%, 98%, or 99% at the target nucleobase pair. The step of contacting may result in in a DNA base editing efficiency of at least about 60%, 61%, 62%, 63%, 64%, 65%, 66%, 67%, 68%, 69%, 70%, 71%, 72%, 73%, 74%, or 75%. In particular, the step of contacting results in on-target base editing efficiencies of greater than 75%, greater than 80%, greater than 85%, greater than 88%, greater than 89%, greater than 90%, or greater than 95%. In certain embodiments, base editing efficiencies of 89% may be realized. In certain embodiments, base editing efficiencies of 99% may be realized.

Some aspects of the disclosure are based on the recognition that any of the base editors described herein are capable of efficiently generating an intended mutation, such as a point mutation, in DNA (e.g. DNA within a genome of a subject) without generating a significant number of unintended mutations, such as unintended point mutations. In some embodiments, an intended mutation is a mutation that is generated by a specific base editor bound to a gRNA, specifically designed to generate the intended mutation (e.g. deamination). In some embodiments, the intended mutation is a mutation associated with a disease or disorder, such as Usher syndrome type 2A.

In some embodiments, the intended mutation is a deamination that generates a stop codon, for example, a premature stop codon within the coding region of a gene. In some embodiments, the intended mutation is a mutation that eliminates a stop codon. In some embodiments, the intended mutation eliminates a stop codon comprising the nucleic acid sequence 5′-TAG-3′, 5′-TAA-3′, or 5′-TGA-3′. Generation of a stop codon in a sequence positioned 5′ of an exon with a disease-causing mutation (e.g., in a subject's or cell's genome) allows the “skipping” of this exon during transcription and/or mRNA processing. Exon skipping may result in generation of a mutated protein that restores ciliogenesis, hair cell morphology, and auditory function in the inner ear (either partially or completely), and/or mitigates early abnormalities in mouse retina (either partially or completely).

In some embodiments, the intended mutation is a deamination that alters the regulatory sequence of a gene (e.g., a gene promoter or gene repressor). In some embodiments, the intended mutation is a deamination introduced into the gene promoter. In particular embodiments, the deamination introduced into the gene promoter leads to a decrease in the transcription of a gene operably linked to the gene promoter. In other embodiments, the deamination leads to an increase in the transcription of a gene operably linked to the gene promoter. In some embodiments, the deamination introduced into a regulatory sequence of the gene, or the gene itself, reverses a frameshift mutation in the USH2A gene that generates a premature stop codon (which results in a truncated USH2A translation product).

In some embodiments, the intended mutation is a deamination that alters the splicing of a gene. Accordingly, in some embodiments, the intended deamination results in the introduction of a splice site in a gene. In other embodiments, the intended deamination results in the removal of a splice site. The introduction of a splice site in a sequence positioned 5′ of an exon with a disease-causing mutation allows the “skipping” of this exon during transcription and/or mRNA processing.

In some embodiments, the target nucleotide sequence is a DNA sequence in a genome, e.g. a eukaryotic genome. In certain embodiments, the target nucleotide sequence is in a mammalian (e.g. a human) genome. In certain embodiments, the target nucleotide sequence is in a human genome. In other embodiments, the target nucleotide sequence is in the genome of a rodent, such as a mouse or a rat. In other embodiments, the target nucleotide sequence is in the genome of a domesticated animal, such as a horse, cat, dog, or rabbit. In some embodiments, the target nucleotide sequence is in the genome of a research animal. In some embodiments, the target nucleotide sequence is in the genome of a genetically engineered non-human subject. In some embodiments, the target nucleotide sequence is in the genome of a plant. In some embodiments, the target nucleotide sequence is in the genome of a microorganism, such as a bacteria.

### Guide RNA Sequences

Some aspects of the invention relate to guide sequences (“guide RNA” or “gRNA”) that are capable of guiding a napDNAbp or a base editor comprising a napDNAbp to a target site in USH2A. In various embodiments base editors (e.g., base editors described herein) can be complexed, bound, or otherwise associated with (e.g., via any type of covalent or non-covalent bond) one or more guide sequences, i.e., the sequence which becomes associated or bound to the base editor and directs its localization to a specific target sequence having complementarity to the guide sequence or a portion thereof. The particular design aspects of a guide sequence will depend upon the nucleotide sequence of a genomic target site of interest (e.g., the C100, G11864, G12575, C13274, or C14803 residues of human USH2A) and the type of napDNA/RNAbp (e.g., type of Cas protein) present in the base editor, among other factors, such as PAM sequence locations, percent G/C content in the target sequence, the degree of microhomology regions, secondary structures, etc. The present disclosure also provides compositions of guide RNAs. The present disclosure also provides methods of editing target DNA sequences in an USH2A gene using compositions and/or complexes comprising any of the disclosed guide RNAs.

In general, a guide sequence is any polynucleotide sequence having sufficient complementarity with a target polynucleotide sequence to hybridize with the target sequence and direct sequence-specific binding of a napDNA/RNAbp (e.g., a Cas9, Cas9 homolog, or Cas9 variant) to the target sequence, such as a sequence within a USH2A gene. In some embodiments, the degree of complementarity between a guide sequence and its corresponding target sequence (e.g., USH2A), when optimally aligned using a suitable alignment algorithm, is about or more than about 50%, 60%, 75%, 80%, 85%, 88%, 89%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 31, 32, 33, 34, 35, 40, 45, 50, or more nucleotides in length.

In some aspects, the present disclosure provides compositions comprising the base editors with reduced RNA editing effects as described herein and one or more guide RNAs, e.g., a single-guide RNA (“sgRNA”). In addition, the present disclosure provides for nucleic acid molecules encoding and/or expressing the base editors as described herein, as well as expression vectors or constructs for expressing the base editors described herein and a gRNA, host cells comprising said nucleic acid molecules and expression vectors, and optionally one or more gRNAs, and compositions for delivering and/or administering nucleic acid-based embodiments described herein.

In some embodiments, a guide sequence is less than about 35, 30, 25, 20, 15, 12, or fewer nucleotides in length. The ability of a guide sequence to direct sequence-specific binding of a base editor to a target sequence may be assessed by any suitable assay. For example, the components of a base editor, including the guide sequence to be tested, may be provided to a host cell having the corresponding target sequence (e.g., a HGADFN 167 or HGADFN 188 cell line), such as by transfection with vectors encoding the components of a base editor disclosed herein, followed by an assessment of preferential cleavage within the target sequence. Similarly, cleavage of a target polynucleotide sequence may be evaluated in a test tube by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments, the guide RNAs for use in accordance with the disclosed methods of editing comprise synthetic single guide RNAs (sgRNAs) containing modified ribonucleotides. In some embodiments, the guide RNAs contain modifications such as 2′-0-methylated nucleotides and phosphorothioate linkages. In some embodiments, the guide RNAs contain 2′-0-methyl modifications in the first three and last three nucleotides, and phosphorothioate bonds between the first three and last three nucleotides. Exemplary modified synthetic sgRNAs are disclosed in Hendel A. et al., Nat. Biotechnol. 33, 985-989 (2015), incorporated herein by reference.

In some embodiments, the guide RNA comprises a structure 5′-[guide sequence]-[Cas9-binding sequence]-3′, where the Cas9-binding sequence (or scaffold) comprises a nucleic acid sequence that is at least 80%, 85%, 90%, 95%, 96%, 97%, 98%, 99%, or 99.5% identical to any one of SEQ ID NOs: 170 and 443-446, or SEQ ID NOs: 170 and 443-446 absent the poly-U terminator sequence at the 3′ end. In some embodiments, the disclosed guide RNA comprises a Cas9 binding sequence comprises a nucleic acid sequence that is at least 80%, 85%, 90%, 95%, 96%, 97%, 98%, 99%, or 99.5% identical to SEQ ID NO: 170. In various embodiments, the Cas9-binding sequence comprises the sequence: 5′-[guide sequence]-gttttagagctagaaatagcaagttaaaataaggctagtccgttatcaacttgaaaaagtggcaccgagtcggtgc-3′ (SEQ ID NO: 170).

In various embodiments, the guide RNA scaffold binds an S. pyogenes Cas9. In other embodiments, the guide RNA scaffold binds an S. aureus Cas9. In some embodiments, the guide RNAs for use in accordance with the disclosed methods of editing comprise a backbone structure that is recognized by an S. pyo genes Cas9 protein or domain, such as an SpCas9 domain of the disclosed base editors. The backbone structure recognized by an SpCas9 protein may comprise the sequence 5′-[guide sequencel-guuuuagagcuagaaauagcaaguuaaaauaaggcuaguccguuaucaacuugaaaaaguggcaccgagucggugcuuu uu-3′ (SEQ ID NO: 443), wherein the guide sequence comprises a sequence that is complementary to the protospacer of the target sequence. See U.S. Publication No. 2015/0166981, published Jun. 18, 2015, the disclosure of which is incorporated by reference herein. In other embodiments, the guide RNAs for use in accordance with the disclosed methods of editing comprise a backbone structure that is recognized by an S. aureus Cas9 protein. The backbone structure recognized by an SaCas9 protein may comprise the sequence 5′-[guide sequence]-guuuuaguacucuguaaugaaaauuacagaaucuacuaaaacaaggcaaaaugccguguuuaucucgucaacuuguugg cgagauuuuuuu-3′ (SEQ ID NO: 444).

In other embodiments, the guide RNAs for use in accordance with the disclosed methods of editing comprise a backbone structure that is recognized by an Lachnospiraceae bacterium Cas12a protein. The backbone structure recognized by an LbCas12a protein may comprise the sequence 5′-[guide sequence]-uaauuucuacuaaguguagau-3′ (SEQ ID NO: 445). In other embodiments, the guide RNAs for use in accordance with the disclosed methods of editing comprise a backbone structure that is recognized by an Acidaminococcus sp. BV3L6 Cas12a protein. The backbone structure recognized by an AsCas12a protein may comprise the sequence 5′-[guide sequence]-uaauuucuacucuuguagau-3′ (SEQ ID NO: 446).

The guide sequence (or spacer) is typically approximately 20 nucleotides long. Examplary spacer and scaffold sequences for the guide RNAs used in the exon-skipping approach are listed in Table 7, below:

TABLE 7
Exon-Skipping Approach Guide Sequences

gRNA Spacer (SEQ ID NOs:
36-63 and 198)
GCUUUAGGGCUUAGGUGUGAU
GGAGcUaaaUUacaaUgaag
GCAGAGGAACcUagagaaga
CCGAAACcUagcaaaUagUa
AUcUaaagcaaaagacaagc
CAGcUgUcaacaaUaaaUgc
UUCACcUgUcaaUUUaggac
gRNA Spacer (SEQ ID NOs:
36-63 and 198)
AGACcUagaaaaagcaagca
AUCCcUUUaaagagagagag
UGUUCCcUgUaagaaaaUUa
GGcUagacaaaaggaagaac
GUAUCUUUAGGGCUUAGGUGU
CCCUAAAGAUAAAAUAUAUU
AAUUAUUCcUagagaaaUUa
aUUUagCACCAGCACAACUG
UGGAGcUgUgaaggaaUaaa
UUUaUUaUagGCAUAUUGAC
UCUUGUGGGGcUgUggaaga
AUUACCUAcUgaUUaaaaaa
AUcUaaagcaaaagacaagc
GGcUagacaaaaggaagaac
CCUUUUUCUUCCgaUcUgUU
CCCcUgagaaggaagUUgcU
CGAAACcUagcaaaUagUaa
CCGAAACcUagcaaaUagUa
GAAACcUagcaaaUagUaag
UACUUCACcUgUcaaUUUag
CAUACUUCACcUgUcaaUUU
ccUgcagAGAUAAUCCAAAA

In some embodiments, the guide sequence comprises a spacer that comprises a nucleic acid sequence that is at least 90%, at least 95%, at least 98%, or at least 99% identical to any one of SEQ ID NOs: 36, 46, 47, 50, 53, 56, 57, and 93. In other embodiments, the guide RNA comprises a nucleic acid sequence that is at least 90%, at least 95%, at least 98%, or at least 99% identical to any one of SEQ ID NOs: 12, 15, 16, 18, 19, 36, 47, and 93. A guide sequence designed for the exon skipping approach of USH2A base editing may comprise any of the sequences of SEQ ID NOs: 36-63 and 92-94. In some embodiments, the guide sequence comprises a nucleic acid sequence that is at least 90%, at least 95%, at least 98%, or at least 99% identical to any one of SEQ ID NOs: 36, 46, 47, 50, 53, 56, 57, and 93. In other embodiments, the guide sequence comprises a nucleic acid sequence that is at least 90%, at least 95%, at least 98%, or at least 99% identical to any one of SEQ ID NOs: 37, 40,

In some embodiments, the guide RNA comprises a nucleic acid sequence that differs by 1, 2, 3, 4, 5, 6, 7, 8, 9, 10 or more than 10 nucleotides from the nucleotide sequences of any one of SEQ ID NOs: 1-63 or 92-94. In some embodiments, the guide RNA comprises a nucleic acid sequence that differs by 1, 2, 3, 4, 5, 6, 7, 8, 9, 10 or more than 10 nucleotides from the nucleotide sequences of any one of SEQ ID NOs: 36, 46, 47, 50, 53, 56, 57, and 93. In other embodiments, the guide RNA comprises a nucleic acid sequence that differs by 1, 2, 3, 4, 5, 6, 7, 8, 9, 10 or more than 10 nucleotides from the nucleotide sequences of any one of any one of SEQ ID NOs: 12, 15, 16, 18, 19, 36, 47, and 93. The guide RNAs may differ by no more than 5 nucleotides from the nucleotide sequences of any one of SEQ ID NOs: 1-63 or 92-94. A guide sequence designed for the exon skipping approach of USH2A base editing may comprise any of the sequences of SEQ ID NOs: 36-63 and 92-94. In some embodiments, the guide sequence comprises a nucleic acid sequence that is at least 90%, at least 95%, at least 98%, or at least 99% identical to any one of SEQ ID NOs: 36, 46, 47, 50, 53, 56, 57, and 93. In other embodiments, the guide sequence comprises a nucleic acid sequence that is at least 90%, at least 95%, at least 98%, or at least 99% identical to any one of SEQ ID NOs: 37, 40, and 44. It should be appreciated, however, that changes to such guide sequences can be made based on the specific USH2A sequence found within a cell, for example the cell of a patient having Usher syndrome or arRP. Such suitable guide RNA sequences typically comprise guide sequences that are complementary to a nucleic sequence within 50 nucleotides upstream or downstream of the target nucleotide to be edited.

Examplary protospacer and scaffold sequences for the guide RNAs used in the mutation-correction approach (cytosine base editing) are listed in Table 8, below:

TABLE 8
Mutation-Correction Approach Guide
Sequences (CBEs) gRNA Spacer

(SEQ ID NOs: 199, 200, 214,
and 201208)
CUCUUACCUUGGGAAAGGAG
UUCCUUCCCGCAUCAGGGAA
UUCCCCGUAAGAAAAUUAAC
AUCCAAAGCAAAAGACAAGC
AUUGGUGAUUCCCGCUGUGG
UAACAGGAAUAUUUGGUAUA
UAGACGCCUCUGCUCCCAGG
AUGAGUCUACUAUUACUCUC
CUUAAACCGGAGAAAACCUA
GGGCAGCGUGAGUGCAAAAA
gRNA Spacer (SEQ ID NOs: 199,
200, 214, and 201208)
CUCUAAAGUAAAAUAAAUCC

Examplary protospacer and scaffold sequences for the guide RNAs used in the mutation-correction approach (adenine base editing) are listed in Table 9, below:

TABLE 9
Mutation-Correction Approach
Guide Sequences (ABEs)

gRNA Spacer (SEQ ID NOs: 209-213,
329, and 215-232)
GUAGUCAUUAACACAAACUC
CAACCAUGCAAGCUUUCAGC
GCUUCAGAGAAAUUUAAAUC
UGAUUCACAGAUGCUUCGAG
GCUCAGUACUGAGGCACUGU
UUCAAAAGCUGGCCUUAAUG
AGACCUCAUGACUCAGUCAA
CCAUUCAUGCAGGCUACAAG
UCCAUUAUCCAGGCAGAUUU
UGUCAGAAGAAUGCAAGAUU
AACCAUGACACUCUAUUAUC
UUGUCAGAAAAAGUAAUCCU
CUACCCUGUCUUAGCAUUAC
ACUAAUUACACCUUCUUCCU
AACUCAACUUUCCUCAGAUG
AACUUCACGGGAGCCCUCCC
UUCGGUAGUUGCAUGAAGGA
AGCAGGUCAUGAGGGUCUUG
UCUAGCCAUGGCCAUCAUGA
GUACAGCAUUCUGCCACAGC
CCACUCAUGCAGGCUUGGAG
AAUUCAUAUUUCAUACCUUC
CGGCAUCUUAACACUUCCUU
CAGGCACUAGCCACUGAUUG

In some embodiments, the guide RNA comprises a nucleic acid sequence that is at least 90%, at least 95%, at least 98%, or at least 99% identical to any one of SEQ ID NOs: 199-232 and 329. In other embodiments, the guide RNA comprises a nucleic acid sequence that comprises any one of SEQ ID NOs: 199-203. In some embodiments, the guide RNA comprises a nucleic acid sequence that differs by 1, 2, 3, 4, 5, 6, 7, 8, 9, 10 or more than 10 nucleotides from the nucleotide sequences of any one of SEQ ID NOs: 199-203. In some embodiments, the guide RNA comprises a nucleic acid sequence that comprises a sequence that is at least 90%, at least 95%, at least 98%, or at least 99% identical to any one of SEQ ID NOs: 199-232 and 329, and comprises a sequence (a scaffold sequence) that is at least 90%, at least 95%, at least 98%, or at least 99% identical to SEQ ID NO: 170. In some embodiments, the guide RNA comprises a nucleic acid sequence that comprises a sequence of any one of SEQ ID NOs: 199-232 and 329, and comprises a sequence (a scaffold sequence) of SEQ ID NO: 170. In various embodiments, the guide RNA comprises the the guide RNA comprises a first nucleic acid sequence that comprises any one of SEQ ID NOs: 199-203 and a second nucleic acid sequence that comprises SEQ NO: 170 without any intervening sequences (e.g., linker sequences). In other embodiments, the guide RNA comprises the the guide RNA comprises a first nucleic acid sequence that comprises any one of SEQ ID NOs: 199-203 and a second nucleic acid sequence that comprises SEQ NO: 170 with an intervening linker sequence. In some embodiments, the guide RNA sequence may be described completely by its spacer (first) sequence and its scaffold (sequence) sequence.

In some embodiments, the the guide RNA comprises a nucleic acid sequence that differs by 1, 2, 3, 4, 5, 6, 7, 8, 9, 10, 11, 12, 13, 14, 15, or more than 15 nucleotides from the nucleotide sequences of SEQ ID NO: 170. In other embodiments, the guide RNA comprises a first nucleic acid sequence that differs by 1, 2, 3, 4, 5, 6, 7, 8, 9, 10, or more than 10 nucleotides from the nucleotide sequences of any one of any one of SEQ ID NOs: 199-232 and 329 and a second nucleic acid sequence that differs by 1, 2, 3, 4, 5, 6, 7, 8, 9, 10, 11, 12, 13, 14, 15, or more than 15 nucleotides from the nucleotide sequences of SEQ ID NO: 170. In certain embodiments, the guide RNA comprises a first sequence that differs by no more than 5 nucleotides from the nucleotide sequences of any one of SEQ ID NOs: 199-232 and 329and a second sequence that differs by no more than 5 nucleotides from the nucleotide sequence of SEQ ID NO: 170. In certain embodiments, the guide RNA comprises any one of SEQ ID NOs: 199-232 and 329 and SEQ ID NO: 170.

Accordingly, in some aspects, the disclosure provides compositions comprising a guide RNA that comprises a first nucleic acid sequence that differs by 1, 2, 3, 4, 5, or 6 nucleotides from the nucleotide sequences of any one of any one of SEQ ID NOs: 36-63, 198, 199-232 and 329, and a second nucleic acid sequence that differs by 1, 2, 3, 4, 5, 6, 7, 8, 9, or 10 nucleotides from the nucleotide sequence of SEQ ID NO: 170. In particular embodiments, the guide RNA of the composition comprises a first nucleic acid sequence comprising any one of SEQ ID NOs: 36-63, 198, 199-232 and 329, and a second nucleic acid sequence comprising SEQ ID NO: 170. In some embodiments, the guide RNA comprises a first nucleic acid sequence comprising SEQ ID NO: 209 and a second nucleic acid sequence comprising SEQ ID NO: 170. In some embodiments, the guide RNA comprises a first nucleic acid sequence comprising SEQ ID NO: 36 and a second nucleic acid sequence comprising SEQ ID NO: 170. The guide RNA may comprise a first sequence comprising SEQ ID NO: 199 and a second sequence comprising SEQ ID NO: 170. The guide RNA may comprise a first nucleic acid sequence of SEQ ID NO: 217 and a second nucleic acid sequence comprising SEQ ID NO: 170.

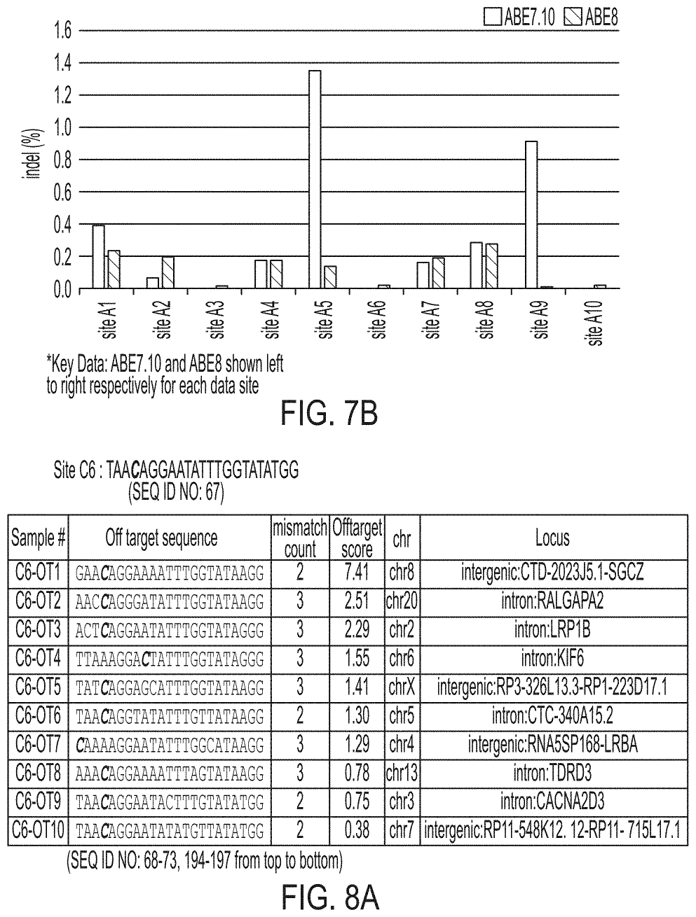

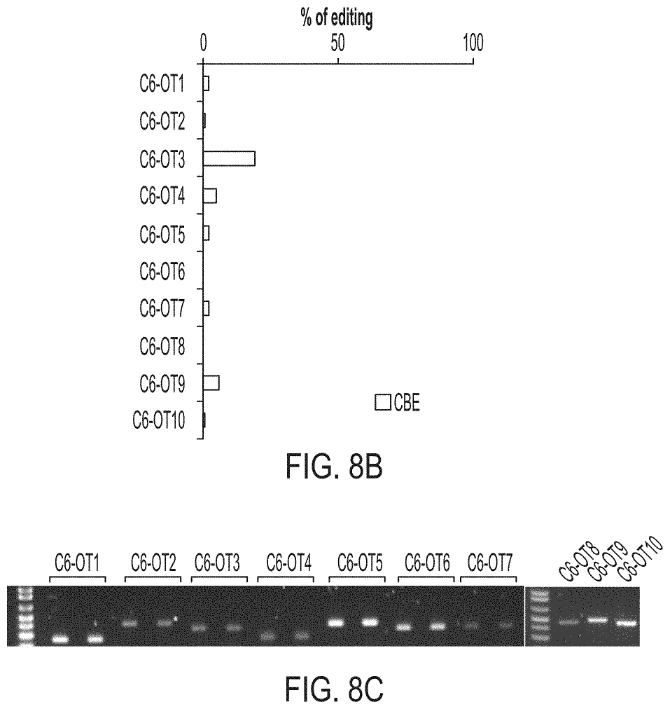

In the muation-correction approach, exemplary guide sequences for targeting a base editor (e.g., ABE or CBE) to an USH2A gene are provided in Tables 4-6 of Example 2 and either of the sequences shown in FIG. 8B. A guide sequence designed for the mutation-correction approach of USH2A base editing may comprise any of the sequences of SEQ ID NOs: 1-35 (as a protospacer).

In the mutation-correction approach, in some embodiments, a guide sequence is is designed to target the C100, G802, C1000, C1876, C2209, C2440, C2755, C2797, C3883, C4645, C4957, G5581, C8167, C9874, C9815, C10712, G11864, G12575, C13010, C13274, C13316, C13822, C14803, or C15017 position in USH2A. In particular embodiments, a guide sequence is designed to target the C100, G11864, G12575, C13274, or C14803 position in USH2A. In some embodiments, a guide sequence is designed to target and correct a G802A, a C1000T, a C1876T, a C2209T, a C2440T, a C2755T, a C2797T, a C3883T, a C4645T, a C4957T, a G5581A, a C8167T, a C9874T, a C9815T, a C10712T, a G11864A, a G12575A, a C13010T, a C13274T, a C13316T, a C13822T, a C14803T, or a C15017T mutation. In some embodiments, provided herein are complexes for adenine base editing that comprise i) any of the disclosed ABEs and ii) one or more of the aforementioned guide sequences. In some embodiments, the administration of any one of these complexes to a subject introduces a point mutation that generates a stop codon, eliminates a stop codon, or alters the regulatory sequence of a gene (e.g., a gene promotor or gene repressor). Particular adenine base editor-gRNA protospacer combinations that are particularly suitable for introducing these mutations are described in Table 5.

Also in the mutation-correction approach, in some embodiments, in some embodiments, a guide sequence is designed to target the T1606, A1841, T2296, A3368, T4325, A7595, A8559, T9799, T10561, or A12067 position in USH2A. In some embodiments, a guide sequence is designed to target and correct a T1606C, an A1841G, a T2296C, an A3368G, a T4325C, an A7595G, an A8559G, a T9799C, a T10561C, or an A12067G mutation. In some embodiments, provided herein are complexes for cytosine base editing that comprise i) any of the disclosed CBEs, and ii) one or more of the aforementioned guide sequences. In some embodiments, the administration of any one of these complexes to a subject introduces a point mutation that generates a stop codon, eleminates a stop codon, or alters the regulatory sequence of a gene (e.g., a gene promotor or gene repressor). Particular cytidine base editor-gRNA protospacer combinations that are particularly suitable for introducing these mutations are described in Table 4.

In the exon-skipping approach, in some embodiments, a guide sequence is designed to target any of the splice acceptor site (SAS) or splice donor site (SDS) positions (or loci) in USH2A shown in Table 6 herein. In particular embodiments, a guide sequence is designed to target the C.2168-2A or C.2168-1G (exon 13) or C.3158-2A or C.3158-1G (exon 16) position in USH2A. In some embodiments, provided herein are complexes for adenine base editing that comprise i) any of the disclosed ABEs, and ii) one or more of the aforementioned guide sequences. In some embodiments, provided herein are complexes for cytidine base editing that comprise i) any of the disclosed CBEs, and ii) one or more of the aforementioned guide sequences. In some embodiments, the administration of any one of these complexes to a subject disrupts a SAS or an SDS of the gene, such as the SAS or SDS of exon 13 or exon 16.

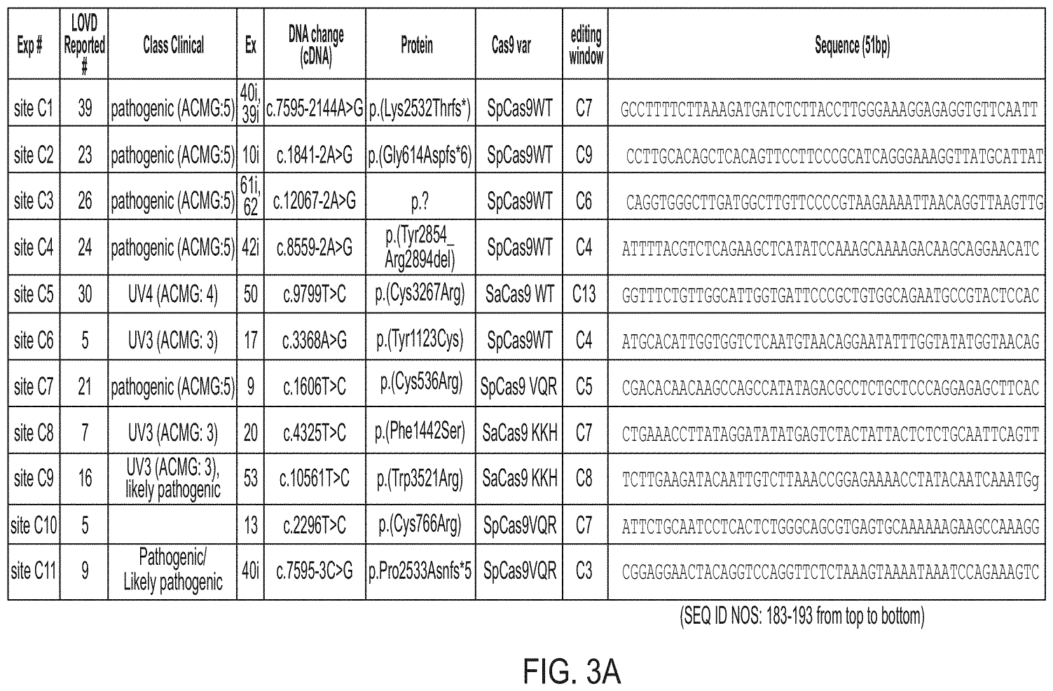

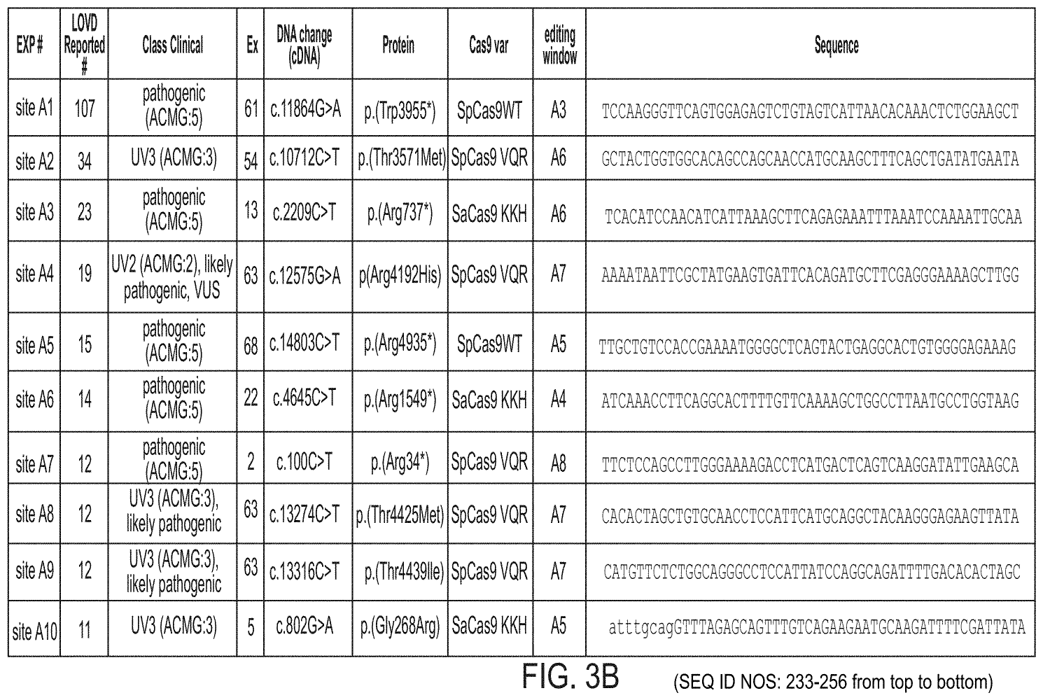

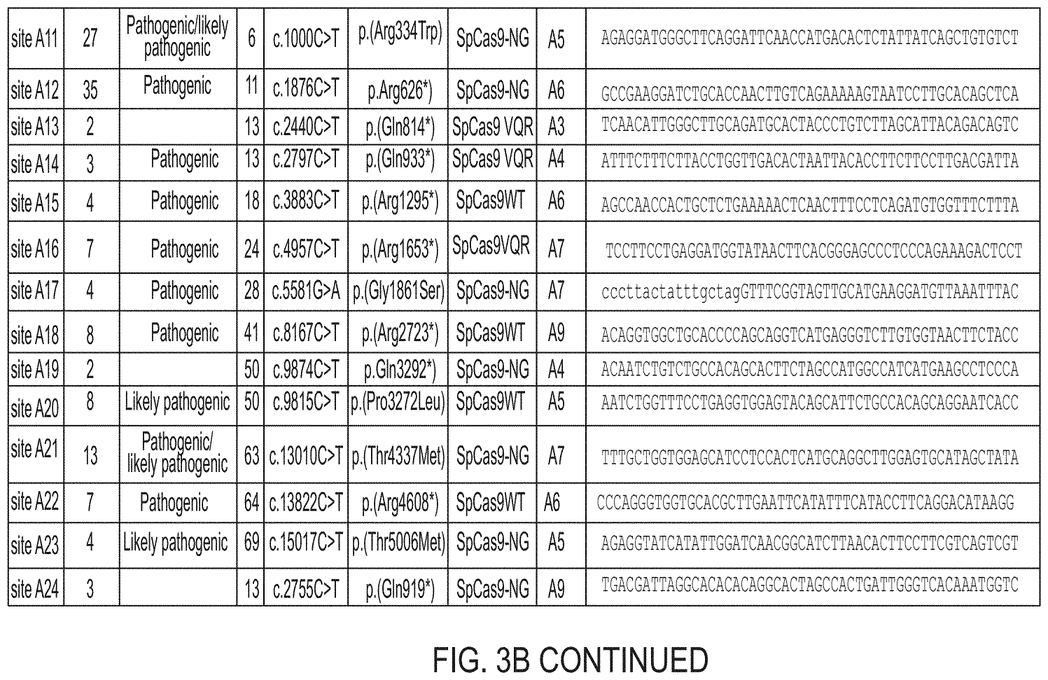

The disclosure contemplates guide sequences that have complementarity to one or more portions of the USH2A gene. It should be appreciated that guide sequences may be engineered that are complementary (e.g., 100% complementary) to any of the exemplary portions of the USH2A gene. In some embodiments, a guide sequence is designed to have complementarity to a target sequence in the USH2A gene that has any of the following mutations: G802A, a C1000T, a C1876T, a C2209T, a C2440T, a C2755T, a C2797T, a C3883T, a C4645T, a C4957T, a G5581A, a C8167T, a C9874T, a C9815T, a C10712T, a G11864A, a G12575A, a C13010T, a C13274T, a C13316T, a C13822T, a C14803T, or a C15017T, T1606C, an A1841G, a T2296C, an A3368G, a T4325C, an A7595G, an A8559G, a T9799C, a T10561C, or an A12067G mutation. The USH2A exons and editing windows associated with each of these mutations are located may be found in FIGS. 3A and 3B.

In some embodiments, a guide sequence is designed to have complementarity to a target sequence in the USH2A gene to any of the splice acceptor sites or splice donor sites in the USH2A gene, e.g., any of the splice acceptor sites or splice donor sites listed in Table 6 of Example 2, below. In particular embodiments, a a guide sequence is designed to have complementarity to a target sequence in the USH2A gene that comprises any of the following splice site loci: C.2168-2A or C.2168-1G (exon 13) or C.3158-2A or C.3158-1G (exon 16) position. In some embodiments, a guide sequence is designed to have complementarity to any of the splice acceptor sites or splice donor sites associated with exon 8, exon 13, exon 16, exon 17, exon 18, exon 21, exon 24, exon 25, exon 28, exon 29, exon 30, exon 31, exon 32, exon 38, exon 43, exon 45, exon 50, exon 53, exon 56, exon 59, exon 62, exon 65, exon 68, exon 69, and exon 71. The precise mutations being introduced in this approach are listed in Table 6 of Example 2. In particular embodiments, a guide sequence is designed to have complementarity to any of the splice acceptor sites or splice donor sites associated with exon 13, exon 16, exon 59, or exon 43. In other embodiments, a guide sequence is designed to have complementarity to any of the splice acceptor sites or splice donor sites associated with exon 17, exon 28, exon 53, or exon 62.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and PA Carr and GM Church, 2009, Nature Biotechnology 27(12): 1151-62). Further algorithms may be found in U.S. application Ser. No. 61/836,080; Broad Reference BI-2013/004A); incorporated herein by reference.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator: (1) NNNNNNNNgtttttgtactctcaagatttaGAAAtaaatcttgcagaagctacaaagataaggctt catgccgaaatcaacaccctgtcattttatggcagggtgttttcgttatttaaTTTTTT (SEQ ID NO: 553); (2) NNNNNNNNNNNNNNNNNNgtttttgtactctcaGAAAtgcagaagctacaaagataaggcttcatgccgaaatca acaccctgtcattttatggcagggtgttttcgttatttaaTTTTTT (SEQ ID NO: 554); (3) NNNNNNNNNNNNNNNNNNNNgtttttgtactctcaGAAAtgcagaagctacaaagataaggcttcatgccgaa atca acaccctgtcattttatggcagggtgtTTTTT (SEQ ID NO: 555); (4) NNNNNNNNNNNNNNNNNNNNgttttagagctaGAAAtagcaagttaaaataaggctagtccgttatcaacttga aaa agtggcaccgagtcggtgcTTTTTT (SEQ ID NO: 556); (5) NNNNNNNNNNNNNNNNNNNNgttttagagctaGAAATAGcaagttaaaataaggctagtccgttatcaactt gaa aaagtgTTTTTTT (SEQ ID NO: 557); and (6) NNNNNNNNNNNNNNNNNNNNgttttagagctagAAATAGcaagttaaaataaggctagtccgttatcaTTT TT TTT (SEQ ID NO: 558). In some embodiments, sequences (1) to (3) are used in combination with Cas9 from S. thermophilus CRISPR. In some embodiments, sequences (4) to (6) are used in combination with Cas9 from S. pyogenes. In some embodiments, the tracr sequence is a separate transcript from a transcript comprising the tracr mate sequence.

It will be apparent to those of skill in the art that in order to target any of the base editors comprising a Cas9 domain and a deaminase, as disclosed herein, to a target site in USH2A to be edited, it is typically necessary to co-express the base editor together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain base editor.

Certain exemplary guide sequences to target USH2A may comprise any of the following guide sequences (spacers). Exemplary guide sequences to target USH2A may comprise any of the guide sequences set forth as SEQ ID NOs: 199-232 and 329.

(SEQ ID NO: 199)
5′-CUCUUACCUUGGGAAAGGAG-3′,
(SEQ ID NO: 200)
5′-UUCCUUCCCGCAUCAGGGAA-3′,
(SEQ ID NO: 214)
5′-UUCCCCGUAAGAAAAUUAAC-3′,
(SEQ ID NO: 201)
5′-AUCCAAAGCAAAAGACAAGC-3′,
(SEQ ID NO: 209)
5′-GUAGUCAUUAACACAAACUC-3′,
(SEQ ID NO: 213)
5′-GCUCAGUACUGAGGCACUGU-3′,
(SEQ ID NO: 215)
5′-UUCAAAAGCUGGCCUUAAUG-3′,
(SEQ ID NO: 216)
5′-AGACCUCAUGACUCAGUCAA-3′,
(SEQ ID NO: 36)
5′-GCUUUAGGGCUUAGGUGUGAU-3′,
(SEQ ID NO: 37)
5′-GGAGCUAAAUUACAAUGAAG-3′,
and
(SEQ ID NO: 217)
5′-CCAUUCAUGCAGGCUACAAG-3′.

In some embodiments, the gRNA comprises a protospacer sequence that comprises any of the nucleic acid sequences as set forth in SEQ ID NOs: 1-35. The protospacer sequence has the same nucleotide sequence as the spacer sequence of the guide RNA, except the spacer sequence comprises ribonucleotides (e.g., uracils), and the protospacer sequence comprises deoxyribonucleotides (e.g., thymines).

In some embodiments, the guide sequence comprises the nucleotide sequence of any one of SEQ ID NOs: 1-63 and 92-94, absent the first 1, 2, 3, 4, 5, 6, 7, 8, 9, or 10 nucleic acid residues from the 5′ end. It should be appreciated that any of the 5′ truncated guide sequences provided herein may further comprise a G residue at the 5′ end. In some embodiments, the guide sequence comprises the amino acid sequence of any one of SEQ ID NOs: 1-63 and 92-94, absent the first 1, 2, 3, 4, 5, 6, 7, 8, 9, 10, 11, or 12 nucleic acid residues from the 3′ end.

The disclosure also provides guide sequences that are longer variants of any of the guide sequences provided herein (e.g., SEQ ID NOs: 36-63, 198, 199-232, and 329). In some embodiments, the guide sequence comprises one additional residue that is 5′-U-3′ at the 3′ end of any one of SEQ ID NOs: 36-63, 198-232, and 329. In some embodiments, the guide sequence comprises two additional residues that are 5′-UG-3′ at the 3′ end of any one of SEQ ID NOs: 36-63, 198-232, and 329. In some embodiments, the guide sequence comprises three additional residues that are 5′-UGG-3′ at the 3′ end of any one of SEQ ID NOs: 36-63, 198-232, and 329. In some embodiments, the guide sequence comprises four additional residues that are 5′-UGGG-3′ at the 3′ end of any one of SEQ ID NOs: 36-63, 198-232, and 329. In some embodiments, the guide sequence comprises five additional residues that are 5′-UGGGA-3′ at the 3′ end of any one of SEQ ID NOs: 1-63 and 92-94. In some embodiments, the guide sequence comprises six additional residues that are 5′-UGGGAA-3′ at the 3′ end of any one of SEQ ID NOs: 36-63, 198-232, and 329. In some embodiments, the guide sequence comprises seven additional residues that are 5′-UGGGAAG-3′ at the 3′ end of any one of SEQ ID NOs: 36-63, 198-232, and 329. In some embodiments, the guide sequence comprises eight additional residues that are 5′-UGGGAAGA-3′ at the 3′ end of any one of SEQ ID NOs: 36-63, 198-232, and 329. In some embodiments, the guide sequence comprises nine additional residues that are 5′-UGGGAAGAA-3′ at the 3′ end of any one of SEQ ID NOs: 36-63, 198-232, and 329.

In some embodiments, the guide RNAs for use in accordance with the disclosed methods of editing comprise synthetic single guide RNAs (sgRNAs) containing modified ribonucleotides. In some embodiments, the guide RNAs contain modifications such as 2′-O-methylated nucleotides and phosphorothioate linkages. In some embodiments, the guide RNAs contain 2′-0-methyl modifications in the first three and last three nucleotides, and phosphorothioate bonds between the first three and last three nucleotides. Exemplary modified synthetic sgRNAs are disclosed in Hendel A. et al., Nat. Biotechnol. 33, 985-989 (2015), herein incorporated by reference.

The sequences of suitable guide RNAs for targeting the disclosed ABEs to specific genomic target sites will be apparent to those of skill in the art based on the present disclosure. Such suitable guide RNA sequences typically comprise guide sequences that are complementary to a nucleic sequence within 50 nucleotides upstream or downstream of the target nucleobase pair to be edited. Some exemplary guide RNA sequences suitable for targeting any of the provided ABEs to specific target sequences are provided herein. Additional guide sequences are are well known in the art and may be used with the base editors described herein. Additional exemplary guide sequences are disclosed in, for example, Jinek M., et al., Science 337:816-821(2012); Mali P, Esvelt K M & Church G M (2013) Cas9 as a versatile tool for engineering biology, Nature Methods, 10, 957-963; Li J F et al., (2013) Multiplex and homologous recombination-mediated genome editing in Arabidopsis and Nicotiana benthamiana using guide RNA and Cas9, Nature Biotechnology, 31, 688-691; Hwang, W. Y. et al., Efficient genome editing in zebrafish using a CRISPR-Cas system, Nature Biotechnology 31, 227-229 (2013); Cong L et al., (2013) Multiplex genome engineering using CRIPSR/Cas systems, Science, 339, 819-823; Cho S W et al., (2013) Targeted genome engineering in human cells with the Cas9 RNA-guided endonuclease, Nature Biotechnology, 31, 230-232; Jinek, M. et al., RNA-programmed genome editing in human cells, eLife 2, e00471 (2013); Dicarlo, J. E. et al., Genome engineering in Saccharomyces cerevisiae using CRISPR-Cas systems. Nucleic Acid Res. (2013); Briner A E et al., (2014) Guide RNA functional modules direct Cas9 activity and orthogonality, Mol Cell, 56, 333-339, the entire contents of each of which are incorporated herein by reference.

BE/gRNA Complexes

Some aspects of this disclosure provide complexes comprising any of the base editors (e.g., base editor) provided herein, for example any of the adenosine base editors described herein, and a guide nucleic acid bound to napDNAbp of the base editor. In some embodiments, the guide nucleic acid is any one of the guide RNAs provided herein. In some embodiments, the disclosure provides any of the base editors (e.g., cytosine base editors or adenosine base editors) provided herein bound to any of the guide RNAs provided herein. In some embodiments, the napDNAbp of the base editor is a Cas9 domain (e.g., a dCas9, a nuclease active Cas9, or a Cas9 nickase), which is bound to a guide RNA. In some embodiments, the complexes provided herein are configured to generate a mutation in a nucleic acid, for example to change an amino acid in a gene (e.g., USH2A) to change the properties of one or more proteins (e.g., Usherin).

In some embodiments, the guide RNA comprises a guide sequence that comprises at least 8, 9, 10, 11, 12, 13, 14, 15, 16, 17, 18, 19, 20, 21, 22, 23, 24, 25, 26, 27, 28, 29, or 30 contiguous nucleic acids that are 100% complementary to a target sequence, for example a target DNA sequence. In some embodiments, the guide RNA comprises a guide sequence that comprises at least 10, 11, 12, 13, 14, 15, 16, 17, 18, 19, 20, 21, 22, 23, 24, or 25 contiguous nucleic acids that are 100% complementary to a DNA sequence in a USH2A gene, for example, a region of a human USH2A gene.

In some embodiments, any of the complexes provided herein comprise a gRNA having a guide sequence that comprises at least 8, 9, 10, 11, 12, 13, 14, 15, 16, 17, 18, 19, 20, 21, 22, 23, 24, 25, 26, 27, 28, 29, or 30 contiguous nucleic acids that are 100% complementary to any one of the nucleic acid sequences provided herein, e.g., the nucleic acid sequences of SEQ ID NOs: 36-63, 198-232, and 329. It should be appreciated that the guide sequence of the gRNA may comprise one or more nucleotides that are not complementary to a target sequence. In some embodiments, the guide sequence of the gRNA is at the 5′ end of the gRNA. In some embodiments, the guide sequence of the gRNA further comprises a G at the 5′ end of the gRNA. In some embodiments, the G at the 5′ end of the gRNA is not complementary with the target sequence. In some embodiments, the guide sequence of the gRNA comprises 1, 2, 3, 4, 5, 6, 7, or 8 nucleotides that are not complementary to a target sequence. In some embodiments, the guide RNA comprises a guide sequence that comprises at least 8, 9, 10, 11, 12, 13, 14, 15, 16, 17, 18, 19, 20, 21, 22, 23, 24, 25, 26, 27, 28, 29, or 30 contiguous nucleic acids that are 100% complementary to a target sequence, for example a target DNA sequence in a USH2A gene. In some embodiments, the guide RNA comprises a guide sequence that comprises at least 10, 11, 12, 13, 14, 15, 16, 17, 18, 19, 20, 21, 22, 23, 24, or 25 contiguous nucleic acids that are 100% complementary to a DNA sequence in a human USH2A gene. In some embodiments, the USH2A gene is a human, chimpanzee, ape, monkey, dog, mouse, or rat USH2A gene. In some embodiments, the USH2A gene is a human USH2A gene.

The present disclosure contemplates the use of any of the guide RNA sequences comprising any of the nucleic acid sequences listed in Tables 7-9 (e.g., SEQ ID NOs: 36-63, 198-232, and 329) in association with any of the disclosed base editors (e.g., the base editors of SEQ ID NOs: 350-388). The present disclosure also contemplates the use of any of the guide RNA sequences comprising any of the protospacer nucleic acid sequences listed in Tables 4 and 5 (e.g., SEQ ID NOs: 1-35) in association with any of the disclosed base editors (e.g., the base editors of SEQ ID NOs: 350-388).

In particular embodiments, the disclosure provides complexes comprising (i) an cytidine base editor, and (ii) a guide RNA (gRNA) comprising a nucleic acid sequence that is at least 90%, at least 95%, at least 98%, or at least 99% identical to any one of SEQ ID NOs: 1-11, 37-45, 48, 49, 51, 52, 54, 55, 58-63, 93, and 198-209. The cytidine base editor may comprise any one of SEQ ID NOs: 350-372, or a variant thereof. Any cytidine base editor in the art is contemplated for use in the complexes of the disclosure. In particular embodiments, the cytidine base editor is BE4max or BE4max-VRQR.

The guide RNA of the disclosed complexes may comprise a nucleic acid sequence selected from the group consisting of SEQ ID NOs: 1-11, 37-45, 48, 49, 51, 52, 54, 55, 58-63, 93, and 198-209. In some embodiments, the guide RNA comprises a nucleic acid sequence that is at least 90%, at least 95%, at least 98%, or at least 99% identical to any one of SEQ ID NOs: 199, 200, 205, 37, 40, and 44. In some embodiments, the gRNA comprises a nucleic acid sequence selected from the group consisting of SEQ ID NOs: 199, 200, 205, 37, 40, and 44. In some embodiments, the complex comprises a base editor selected from BE3, BE3.9max, BE4max, BE4-SaKKH, BE3.9-NG, BE3.9-NRRH, or BE4max-VRQR. In particular embodiments, the complex comprises BE4max and a gRNA that comprises the nucleic acid sequence of SEQ ID NO: 199. In other embodiments, the cytidine base editor is BE4max, and the gRNA comprises the nucleic acid sequence of SEQ ID NO: 40. The complex may comprise a cytidine base editor that comprises BE4max and a gRNA that comprises a first nucleic acid sequence of SEQ ID NO: 199 and a second nucleic acid sequence comprising SEQ ID NO: 170.

In other embodiments, the disclosure provides complexes comprising (i) an adenosine base editor, and (ii) a guide RNA (gRNA) comprises a nucleic acid sequence that is at least 90%, at least 95%, at least 98%, or at least 99% identical to any one of SEQ ID NOs: 12-36, 46, 47, 50, 53, 56, 57, 93, 209-213, 215-232, and 329. The guide RNA of the complexes may comprise a nucleic acid sequence selected from the group consisting of SEQ ID NOs: 12-36, 46, 47, 50, 53, 56, 57, 93, 209-213, 215-232, and 329. The adenine base editor may comprise any one of SEQ ID NOs: 373-388, or a variant thereof. Any adenine base editor in the art is contemplated for use in the complexes of the disclosure. In some embodiments, the adenine base editor is ABE7.10, ABE8e, ABE8e-SaKKH, ABE8e-NG, ABE-xCas9, ABE7.10-SaKKH, ABE7.10-NG, ABE7.10-VRQR, ABE8e-NRTH, ABE8e-NRRH, or ABE8e-VRQR. In particular embodiments, the adenine base editor is ABE8e.

In some embodiments, the guide RNA of the complex comprises a protospacer that comprises a nucleic acid sequence that is at least 90%, at least 95%, at least 98%, or at least 99% identical to any one of SEQ ID NOs: 209, 213, 216, 217, 36, and 47. In some embodiments, the guide RNA of the complex comprises a protospacer that comprises the nucleic acid sequence of any one of SEQ ID NOs: 209, 213, 216, 217, 36, and 47. In particular embodiments, the complex comprises an adenine base editor that comprises ABE8e, and a guide RNA that comprises a first nucleic acid sequence of SEQ ID NO: 217 and a second nucleic acid sequence comprising SEQ ID NO: 170. The complex may comprise ABE8e-VRQR and a guide RNA comprising a first nucleic acid sequence of SEQ ID NO: 209 and a second nucleic acid sequence comprising SEQ ID NO: 170.

Any of the above-disclosed complexes may be comprised within a pharmaceutical composition, a host cell, one or more vectors, and one ore more rAAV vectors.

### Editing Methods

Some aspects of this disclosure provide methods of using the base editors, or complexes comprising a guide nucleic acid (e.g., gRNA) and a base editor described herein. For example, some aspects of this disclosure provide methods comprising contacting a DNA, or RNA molecule with any of the base editors described herein, and with at least one guide nucleic acid (e.g., guide RNA), wherein the guide nucleic acid, (e.g., guide RNA) is comprises a sequence (e.g., a guide sequence that binds to a DNA target sequence) of at least 10 (e.g., at least 10, 15, 20, 25, or 30) contiguous nucleotides that is 100% complementary to a target sequence (e.g., any of the target USH2A sequences provided herein). In some embodiments, the 3′ end of the target sequence is immediately adjacent to a canonical PAM sequence (NGG). In some embodiments, the 3′ end of the target sequence is not immediately adjacent to a canonical PAM sequence (NGG). In some embodiments, the 3′ end of the target sequence is immediately adjacent to an AGC, GAG, TTT, GTG, or CAA sequence.

Some aspects of the disclosure provide methods of using base editors (e.g., any of the base editors described herein) and gRNAs to change a residue in a USH2A gene. In some embodiments, the disclosure provides methods of using base editors (e.g., any of the base editors described herein) and gRNAs to generate an A to G and/or T to C mutation in an USH2A gene, thereby resulting in a non-functional Usherin protein. In some embodiments, the disclosure provides method for deaminating an adenosine nucleobase (A) in an USH2A gene, the method comprising contacting the USH2A gene with a base editor and a guide RNA bound to the base editor, where the guide RNA comprises a guide sequence that is complementary to a target nucleic acid sequence in the USH2A gene.

In some embodiments, the USH2A gene comprises a C to T or G to A mutation. In some embodiments, the C to T or G to A mutation in the USH2A gene impairs function of the Usherin protein encoded by the USH2A gene. In particular, the mutated Usherin protein may be truncated and/or non-functional. In some embodiments of the disclosed editing methods, deaminating a mutant adenosine (A) nucleobase, or deaminating the A nucleobase complementary to the mutant thymine (T) nucleobase, corrects the C to T or G to A mutation in the USH2A gene. In some embodiments, correction of the C to T or G to A mutation in the USH2A gene leads to an amino acid substitution in the Usherin protein encoded by the USH2A gene. Exemplary amino acid substitutions in the USH2A protein that the presently disclosed adenine base editing methods aim to correct, along with their associated pathogenicities, are presented in Table 5 and FIGS. 3A and 3B.

In other embodiments, the USH2A gene comprises a T to C or A to G mutation. In some embodiments, the T to C or A to G mutation in the USH2A gene impairs function of the Usherin protein encoded by the USH2A gene. In particular, the mutated Usherin protein may be truncated and/or non-functional. In some embodiments of the disclosed editing methods, deaminating a mutant cytosine (C) nucleobase, or deaminating a C nucleobase complementary to the mutant guanine (G) nucleobase, corrects the T to C or A to G mutation in the USH2A gene. In some embodiments, correction of the T to C or A to G mutation in the USH2A gene leads to an amino acid substitution in the Usherin protein encoded by the USH2A gene. Exemplary amino acid substitutions in the USH2A protein that the presently disclosed cytidine base editing methods aim to correct, along with their associated pathogenicities, are presented in Table 4 and FIGS. 3A and 3B .

In some embodiments, the USH2A gene comprises a (wild-type) T or A nucleobase at a splice acceptor site or a splice donor site. In some embodiments of the disclosed editing methods, deaminating the adenosine (A) nucleobase, or deaminating the A nucleobase complementary to the thymine (T) nucleobase, induces exon skipping that restores (partially or completely) a functional Usherin protein. In other embodiments, the USH2A gene comprises a (wild-type) C or G nucleobase at a splice acceptor site or a splice donor site. In some embodiments of the disclosed editing methods, deaminating the cytosine (C) nucleobase, or deaminating a C nucleobase complementary to the guanine (G) nucleobase, in the deamination of a cytidine nucleobase in the USH2A gene that induces exon skipping that restores (partially or completely) a functional Usherin protein.

In some embodiments, the guide sequence of the gRNA comprises at least 8, 9, 10, 11, 12, 13, 14, 15, 16, 17, 18, 19, 20, 21, 22, 23, 24, 25, 26, 27, 28, 29, 30, 31, 32, 33, 34, or 35 contiguous nucleic acids that are 100% complementary to a target nucleic acid sequence of the USH2A gene. In some embodiments, the base editor nicks the target sequence that is complementary to the guide sequence.

In some embodiments, the target DNA sequence comprises a sequence associated with a USH2A pathway disease or disorder, e.g., Usher syndrome or arRP. In some embodiments, the target DNA sequence comprises a point mutation associated with a specific amino acid of USH2A. In some embodiments, the activity of the base editor (e.g., comprising an adenosine deaminase and a Cas9 domain), or the complex, results in a non-functional Usherin protein. In some embodiments, the target DNA sequence encodes a protein, and the point mutation is in a codon and results in a change in the amino acid encoded by the mutant codon as compared to the wild-type codon. In some embodiments, the deamination of the mutant A results in a change of the amino acid encoded by the mutant codon. In some embodiments, the deamination of the mutant A results in the codon encoding the mutant amino acid. In some embodiments, the contacting is in vivo in a subject. In some embodiments, the subject has or has been diagnosed with Usher syndrome.

Some embodiments provide methods for using the DNA editing base editors described herein. In some embodiments, the base editor is used to introduce a point mutation into a nucleic acid by deaminating a target nucleobase, e.g., an A residue.

The present disclosure provides methods for the treatment of a subject diagnosed with a disease associated with USH2A that can be corrected by a DNA editing base editor described herein. For example, in some embodiments, a method is provided that comprises administering to a subject having such a disease, e.g., Usher syndrome or arRP.

In some embodiments, a base editor recognizes canonical PAMs and therefore can correct the pathogenic G to A or C to T mutations with canonical PAMs, e.g., NGG, respectively, in the flanking sequences. For example, Cas9 proteins that recognize canonical PAMs comprise an amino acid sequence that is at least 80%, 85%, 90%, 95%, 97%, 98%, or 99% identical to the amino acid sequence of Streptococcus pyogenes Cas9 as provided by any one of SEQ ID NOs: 74, 77, and 79, or to a fragment thereof comprising the RuvC and HNH domains of any one of SEQ ID NO: 74, 102, and 107.

Some aspects of the disclosure provide methods for editing a nucleic acid. In some embodiments, the method is a method for editing a nucleobase of a nucleic acid (e.g., a base pair of a double-stranded DNA sequence). In some embodiments, the method comprises the steps of: a) contacting a target region of a nucleic acid (e.g., a double-stranded DNA sequence) with a complex comprising a base editor (e.g., a Cas9 domain fused to an adenosine deaminase) and a guide nucleic acid (e.g., gRNA), wherein the target region comprises a targeted nucleobase pair, b) inducing strand separation of said target region, c) converting a first nucleobase of said target nucleobase pair in a single strand of the target region to a second nucleobase, and d) cutting no more than one strand of said target region, where a third nucleobase complementary to the first nucleobase base is replaced by a fourth nucleobase complementary to the second nucleobase. In some embodiments, the method results in less than 20% indel formation in the nucleic acid. It should be appreciated that in some embodiments, step b is omitted. In some embodiments, the first nucleobase is an adenine. In some embodiments, the second nucleobase is a deaminated adenine, or inosine. In some embodiments, the third nucleobase is a thymine. In some embodiments, the fourth nucleobase is a cytosine. In some embodiments, the method results in less than 19%, 18%, 16%, 14%, 12%, 10%, 8%, 6%, 4%, 2%, 1%, 0.5%, 0.2%, or less than 0.1% indel formation. In some embodiments, the method further comprises replacing the second nucleobase with a fifth nucleobase that is complementary to the fourth nucleobase, thereby generating an intended edited base pair (e.g., A:T to G:C). In some embodiments, the fifth nucleobase is a guanine. In some embodiments, at least 5% of the intended base pairs are edited. In some embodiments, at least 10%, 15%, 20%, 25%, 30%, 35%, 40%, 45%, or 50% of the intended base pairs are edited.

In some embodiments, the ratio of intended products to unintended products in the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended point mutation to indel formation is greater than 1:1, 10:1, 50:1, 100:1, 500:1, or 1000:1, or more. In some embodiments, the cut single strand (nicked strand) is hybridized to the guide nucleic acid. In some embodiments, the cut single strand is opposite to the strand comprising the first nucleobase. In some embodiments, the base editor comprises a Cas9 domain. In some embodiments, the first base is adenine, and the second base is not a G, C, A, or T. In some embodiments, the second base is inosine. In some embodiments, the first base is adenine. In some embodiments, the second base is not a G, C, A, or T. In some embodiments, the second base is inosine. In some embodiments, the base editor inhibits base excision repair of the edited strand. In some embodiments, the base editor protects or binds the non-edited strand. In some embodiments, the base editor comprises UGI activity. In some embodiments, the base editor comprises a catalytically inactive inosine-specific nuclease. In some embodiments, the base editor comprises nickase activity. In some embodiments, the intended edited base pair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited base pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the base editor comprises a linker. In some embodiments, the linker is 1-25 amino acids in length. In some embodiments, the linker is 5-20 amino acids in length. In some embodiments, linker is 10, 11, 12, 13, 14, 15, 16, 17, 18, 19, or 20 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair is within the target window. In some embodiments, the target window comprises the intended edited base pair. In some embodiments, the method is performed using any of the base editors described herein. In some embodiments, a target window is a deamination window.

In some embodiments, the disclosure provides methods for editing a nucleotide. In some embodiments, the disclosure provides a method for editing a nucleobase pair of a double-stranded DNA sequence. In some embodiments, the method comprises a) contacting a target region of the double-stranded DNA sequence with a complex comprising a base editor and a guide nucleic acid (e.g., gRNA), where the target region comprises a target nucleobase pair, b) inducing strand separation of said target region, c) converting a first nucleobase of said target nucleobase pair in a single strand of the target region to a second nucleobase, d) cutting no more than one strand of said target region, wherein a third nucleobase complementary to the first nucleobase base is replaced by a fourth nucleobase complementary to the second nucleobase, and the second nucleobase is replaced with a fifth nucleobase that is complementary to the fourth nucleobase, thereby generating an intended edited base pair, wherein the efficiency of generating the intended edited base pair is at least 5%. It should be appreciated that in some embodiments, step b is omitted. In some embodiments, at least 5% of the intended base pairs are edited. In some embodiments, at least 10%, 15%, 20%, 25%, 30%, 35%, 40%, 45%, or 50% of the intended base pairs are edited. In some embodiments, the method causes less than 19%, 18%, 16%, 14%, 12%, 10%, 8%, 6%, 4%, 2%, 1%, 0.5%, 0.2%, or less than 0.1% indel formation. In some embodiments, the ratio of intended product to unintended products at the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended point mutation to indel formation is greater than 1:1, 10:1, 50:1, 100:1, 500:1, or 1000:1, or more. In some embodiments, the cut single strand is hybridized to the guide nucleic acid. In some embodiments, the cut single strand is opposite to the strand comprising the first nucleobase. In some embodiments, the first base is adenine. In some embodiments, the second nucleobase is not G, C, A, or T. In some embodiments, the second base is inosine. In some embodiments, the base editor inhibits base excision repair of the edited strand. In some embodiments, the base editor protects (e.g., form base excision repair) or binds the non-edited strand. In some embodiments, the base editor comprises UGI activity. In some embodiments, the base editor comprises a catalytically inactive inosine-specific nuclease. In some embodiments, the base editor comprises nickase activity. In some embodiments, the intended edited base pair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited base pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the base editor comprises a linker. In some embodiments, the linker is 1-25 amino acids in length. In some embodiments, the linker is 5-20 amino acids in length. In some embodiments, the linker is 10, 11, 12, 13, 14, 15, 16, 17, 18, 19, or 20 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair occurs within the target window. In some embodiments, the target window comprises the intended edited base pair. In some embodiments, the base editor is any one of the base editors described herein.

The present disclosure provides methods for the treatment of a subject diagnosed with a disease associated with USH2A, e.g., Usher syndrome or arRP. For example, in some embodiments, a method is provided that comprises administering to a subject having such a disease, e.g., Usher syndrome, type 2A, an effective amount of a complex comprising any of the the base editors and guide RNAs described herein.

Any of the complexes disclosed in the section above may be used in the mutation-correction approach. Particular complexes of base editors and guide RNA sequences that are useful for mutation-correction approach are listed in Tables 4 and 5. Particularly useful complexes of ABEs and guide RNA sequences are listed in Table 5, and particularly useful complexes of CBEs and guide RNA sequences are listed in Table 4. Any of the complexes comprising CBEs disclosed herein (e.g., the CBEs of SEQ ID NOs: 350-372) may be used to correct any of the following mutations: a T1606C, an A1841G, a T2296C, an A3368G, a T4325C, an A7595G, an A8559G, a T9799C, a T10561C, and an A12067G mutation.

In particular embodiments, methods of editing are provided wherein the guide RNA comprises a first nucleic acid sequence comprising SEQ ID NO: 199 and a second nucleic acid sequence comprising SEQ ID NO: 170, wherein the base editor is BE4max, and wherein the USH2A gene comprises an A7595G mutation. Any of the complexes comprising ABEs disclosed herein (e.g., the ABEs of SEQ ID NOs: 373-388) may be used to correct any of the following mutations: a G802A, a C1000T, a C1876T, a C2209T, a C2440T, a C2755T, a C2797T, a C3883T, a C4645T, a C4957T, a G5581A, a C8167T, a C9874T, a C9815T, a C10712T, a G11864A, a G12575A, a C13010T, a C13274T, a C13316T, a C13822T, a C14803T, and a C15017T mutation. In particular embodiments, methods of editing are provided wherein the guide RNA comprises a first nucleic acid sequence comprising SEQ ID NO: 209 and a second nucleic acid sequence comprising SEQ ID NO: 170, wherein the base editor is ABE8e, and wherein the USH2A gene comprises a G11864A mutation.

Any of the complexes disclosed in the section above may be used in the exon skipping approach, which involved disruption of a splice acceptor site or a splice donor site. Particular complexes of base editors and guide RNA sequences that are useful for exon skipping approach, along with particular SAS or SDS positions (or loci) in USH2A of interest shown in Table 6 herein. The base editor-gRNA combinations are listed in Table 6 next to the SAS or SDS positions for which these combinations are particularly useful for editing to induce exon skipping. (The exon associated with each SAS and SDS is also listed in Table 6.) Any of the complexes comprising CBEs disclosed herein (e.g., the CBEs of SEQ ID NOs: 350-372) may be used to disrupt a splice acceptor site or a splice donor site in the USH2A gene associated with exon 8, exon 13, exon 16, exon 17, exon 18, exon 21, exon 24, exon 28, exon 29, exon 30, exon 31, exon 32, exon 38, exon 43, exon 45, exon 50, exon 53, exon 56, exon 59, exon 62, exon 65, exon 68, exon 69, and exon 71. In particular embodiments, methods of editing are provided wherein the guide RNA comprises a first nucleic acid sequence comprising SEQ ID NO: 199 and a second nucleic acid sequence comprising SEQ ID NO: 170, wherein the base editor is BE4max, and wherein the USH2A gene comprises an A7595G mutation.

And any of the complexes comprising ABEs disclosed herein (e.g., the ABEs of SEQ ID NOs: 373-388) may be used to disrupt a splice acceptor site or a splice donor site associated with exon 13, exon 21, exon 25, exon 31, and exon 69. In particular embodiments, methods of editing are provided wherein the guide RNA comprises a first nucleic acid sequence comprising SEQ ID NO: 36 and a second nucleic acid sequence comprising SEQ ID NO: 170, wherein the base editor is an ABE8e, and wherein the step of contacting results in the disruption of a splice acceptor site or a splice donor site associated with exon 13.

### Base Editor Delivery

In some aspects, the present disclosure provides for the delivery of base editors in vitro and in vivo using various strategies, including on separate vectors using split inteins and as well as direct delivery strategies of the ribonucleoprotein complex (i.e., the base editor complexed to the gRNA and/or the second-site gRNA) using techniques such as electroporation, use of cationic lipid-mediated formulations, and induced endocytosis methods using receptor ligands fused to the ribonucleoprotein complexes. In addition, mRNA delivery methods may also be employed. Any such methods are contemplated herein.

Such methods may involve transducing (e.g., via transfection) cells with a plurality of complexes each comprising a base editor and a gRNA molecule. In some embodiments, the gRNA is bound to the napDNAbp domain (e.g., nCas9 domain) of the base editor. In some embodiments, each gRNA comprises a guide sequence of at least 10 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that is complementary to a target sequence. In certain embodiments, the methods involve the transfection of nucleic acid constructs (e.g., plasmids and mRNA constructs) that each (or together) encode the components of a complex of base editor and gRNA molecule. In certain embodiments, any of the disclosed base editors and a gRNA are administered as a protein:RNA complex, such as a ribonucleoprotein complex. In some embodiments, any of the disclosed base editors are administered as an mRNA construct, along with the gRNA molecule. In particular embodiments, administration to cells is achieved by electroporation or lipofection.

In certain embodiments of the disclosed methods, a nucleic acid construct (e.g., a plasmid or an mRNA construct) that encodes the base editor (BE) is transfected into the cell separately from the nucleic acid construct that encodes the gRNA molecule. In certain embodiments, these components are encoded on a single construct and transfected together. In other embodiments, the methods disclosed herein involve the introduction into cells of a complex comprising a BE and a gRNA molecule that has been expressed and cloned outside of these cells.

It should be appreciated that any of the base editors described herein, may be introduced into the cell in any suitable way, either stably or transiently. In some embodiments, a base editor may be transfected into the cell. In some embodiments, the cell may be transduced or transfected with a nucleic acid construct that encodes a base editor. For example, a cell may be transduced (e.g., with a virus encoding a base editor) with a nucleic acid that encodes a base editor, or the translated base editor. As an additional example, a cell may be transfected with a nucleic acid (e.g., a plasmid) that encodes a base editor or the translated base editor. Such transductions or transfections may be stable or transient. In some embodiments, cells expressing a base editor or containing a base editor may be transduced or transfected with one or more gRNA molecules, for example when the base editor comprises a Cas9 (e.g., nCas9) domain. In some embodiments, a plasmid expressing a base editor may be introduced into cells through electroporation, transient transfection (e.g., lipofection, such as with Lipofectamine 3000®), stable genome integration (e.g., piggybac), viral transduction, or other methods known to those of skill in the art.

In some aspects, the invention provides methods comprising delivering one or more base editor-encoding polynucleotides, such as or one or more vectors as described herein encoding one or more components of the base editing system described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology, Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™) Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral-based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and w2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. Reference is made to US 2003/0087817, published May 8, 2003, International Patent Application No. WO 2016/205764, published Dec. 22, 2016, International Patent Application No. WO 2018/071868, published Apr. 19, 2018, U.S. Patent Publication No. 2018/0127780, published May 10, 2018, and International Publication No. WO2020/236982, published Nov. 26, 2020, the disclosures of each of which are incorporated herein by reference.

In various embodiments, the base editor constructs (including, the split-constructs) may be engineered for delivery in one or more rAAV vectors. An rAAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). In other embodiments, the rAAV may be an rAAV1, rAAV2, rAAV3, rAAV4, rAAV5, rAAV6, rAAV7, rAAV8, rAAV9, rAAV10, rAAV12, or rAAV12 vector or particle. In certain embodiments, the discosed rAAV is of serotype 9, i.e., AAV9. An rAAV may comprise a genetic load (i.e., a recombinant nucleic acid vector that expresses a gene of interest, such as a whole or split base editor that is carried by the rAAV into a cell) that is to be delivered to a cell. An rAAV may be chimeric.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVrh.74, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AAVS and VP1u of AAV1. Other non-limiting example of derivatives and pseudotypes that have chimeric VP1 proteins are rAAV2/5-8VP1u, rAAV2/9-1VP1u, and rAAV2/9-8VP1u.

AAV derivatives/pseudotypes, and methods of producing such derivatives/pseudotypes are known in the art (see, e.g., Mol Ther. 2012 Apr; 20(4):699-708. doi: 10.1038/mt.2011.287. Epub 2012 Jan 24. The AAV vector toolkit: poised at the clinical crossroads. Asokan A L, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

Recombinant AAV may comprise a nucleic acid vector, which may comprise at a minimum: (a) one or more heterologous nucleic acid regions comprising a sequence encoding a protein or polypeptide of interest or an RNA of interest (e.g., a siRNA or microRNA), and (b) one or more regions comprising inverted terminal repeat (ITR) sequences (e.g., wild-type ITR sequences or engineered ITR sequences) flanking the one or more nucleic acid regions (e.g., heterologous nucleic acid regions). Herein, heterologous nucleic acid regions comprising a sequence encoding a protein of interest or RNA of interest are referred to as genes of interest.

Any one of the rAAV particles provided herein may have capsid proteins that have amino acids of different serotypes outside of the VP1u region. In some embodiments, the serotype of the backbone of the VP1 protein is different from the serotype of the ITRs and/or the Rep gene. In some embodiments, the serotype of the backbone of the VP1 capsid protein of a particle is the same as the serotype of the ITRs. In some embodiments, the serotype of the backbone of the VP1 capsid protein of a particle is the same as the serotype of the Rep gene. In some embodiments, capsid proteins of rAAV particles comprise amino acid mutations that result in improved transduction efficiency.

In some embodiments, the nucleic acid vector comprises one or more regions comprising a sequence that facilitates expression of the nucleic acid (e.g., the heterologous nucleic acid), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

Final AAV constructs may incorporate a sequence encoding the gRNA. In other embodiments, the AAV constructs may incorporate a sequence encoding the second-site nicking guide RNA. In still other embodiments, the AAV constructs may incorporate a sequence encoding the second-site nicking guide RNA and a sequence encoding the gRNA.

In various embodiments, the gRNAs and the second-site nicking guide RNAs can be expressed from an appropriate promoter, such as a human U6 (hU6) promoter, a mouse U6 (mU6) promoter, or other appropriate promoter. The gRNAs and the second-site nicking guide RNAs can be driven by the same promoters or different promoters.

In some embodiments, a rAAV constructs or the herein compositions are administered to a subject enterally. In some embodiments, a rAAV constructs or the herein compositions are administered to the subject parenterally. In some embodiments, a rAAV particle or the herein compositions are administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs.

In some embodiments, any of the disclosed rAAV vectors or particles comprising same are administered subretinally. In particular embodiments, any of the disclosed rAAV vectors or particles comprising same are administered subretinally to the eye of a subject. Any of the disclosed rAAV particles may be administered by injection to the inner ear of a subject. In particular embodiments, the disclosed rAAV vectors are formulated for delivery to a retina or an inner ear of the subject by injection.

In other aspects, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning base editor.

These split intein-based methods overcome several barriers to in vivo delivery. For example, the DNA encoding base editors is larger than the rAAV packaging limit, and so requires special solutions. One such solution is formulating the editor fused to split intein pairs that are packaged into two separate rAAV particles that, when co-delivered to a cell, reconstitute the functional editor protein.

In this aspect, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning base editor.

In various embodiments, the base editors may be engineered as two half proteins (i.e., a BE N-terminal half and a BE C-terminal half) by “splitting” the whole base editor as a “split site.” The “split site” refers to the location of insertion of split intein sequences (i.e., the N intein and the C intein) between two adjacent amino acid residues in the base editor. More specifically, the “split site” refers to the location of dividing the whole base editor into two separate halves, wherein in each halve is fused at the split site to either the N intein or the C intein motifs. The split site can be at any suitable location in the base editor, but preferably the split site is located at a position that allows for the formation of two half proteins which are appropriately sized for delivery (e.g., by expression vector) and wherein the inteins, which are fused to each half protein at the split site termini, are available to sufficiently interact with one another when one half protein contacts the other half protein inside the cell. In particular embodiments, the split site is at an amino acid residue within give or fewer positions of residue 638 of the napDNAbp domain. In particular embodiments, the split site is at residue 574 or residue 638 of the napDNAbp domain (e.g., an SpCas9 domain) of any of the disclosed base editors. In particular embodiments, the split site is immediately before Cys 574 or before Thr 638 (i.e., immediately after residue 573 or after residue 637) of an SpCas9 nickase domain (SEQ ID NO: 107) of the base editor. In other embodiments, the split site is immediately before Cys 574 or before Thr 638 (i.e., immediately after residue 573 or after residue 637) of an SpCas9 wild-type domain (SEQ ID NO: 74) of the base editor.

In some embodiments, the split site is located in the napDNAbp domain. In other embodiments, the split site is located in the RT domain. In other embodiments, the split site is located in a linker that joins the napDNAbp domain and the RT domain.

In various embodiments, split site design requires finding sites to split and insert an N- and C-terminal intein that are both structurally permissive for purposes of packaging the two half base editor domains into two different AAV genomes. Additionally, intein residues necessary for trans splicing can be incorporated by mutating residues at the N terminus of the C terminal extein or inserting residues that will leave an intein “scar.” In various embodiments, using SpCas9 nickase (SEQ ID NO: 74, 1368 amino acids) as an example, the split can be between any two amino acids between 1 and 1368. Preferred splits, however, will be located between the central region of the protein, e.g., from amino acids 50-1250, or from 100-1200, or from 150-1150, or from 200-1100, or from 250-1050, or from 300-1000, or from 350-950, or from 400-900, or from 450-850, or from 500-800, or from 550-750, or from 600-700 of SEQ ID NO: 74. In specific exemplary embodiments, the split site may be between 740/741, or 801/802, or 1010/1011, or 1041/1042. In other embodiments the split site may be between 1/2, 2/3, 3/4, 4/5, 5/6, 6/7, 7/8, 8/9, 9/10, 10/11, 12/13, 14/15, 15/16, 17/18, 19/20 . . . 50/51 . . . 100/101 . . . 200/201 . . . 300/301 . . . 400/401 . . . 500/501 . . . 600/601 . . . 700/701 . . . 800/801 . . . 900/901 . . . 1000/1001 . . . 1100/1101 . . . 1200/1201 . . . 1300/1301 . . . and 1367/1368, including all adjacent pairs of amino acid residues.

In various embodiments, the split inteins can be used to separately deliver separate portions of a complete base editor to a cell, which upon expression in a cell, become reconstituted as a complete base editor through the trans splicing.

In some embodiments, the disclosure provides a method of delivering a base editor to a cell, comprising: constructing a first expression vector encoding an N-terminal fragment of the base editor fused to a first split intein sequence; constructing a second expression vector encoding a C-terminal fragment of the base editor fused to a second split intein sequence; delivering the first and second expression vectors to a cell, wherein the N-terminal and C-terminal fragment are reconstituted as the base editor in the cell as a result of trans splicing activity causing self-excision of the first and second split intein sequences.

In other embodiments, the split site is in the napDNAbp domain. In still other embodiments, the split site is in the cytosine deaminase or adenosine deaminase domain. In yet other embodiments, the split site is in the linker.

Accordingly, in some aspects, the disclosure provides compositions comprising: (i) a first recombinant adeno associated virus (rAAV) particle comprising a first nucleotide sequence (or vector) encoding a N-terminal portion of any of the disclosed base editors fused at its C-terminus to an intein-N; and (ii) a second recombinant adeno associated virus (rAAV) particle comprising a second nucleotide sequence (or vector) encoding an intein-C fused to the N-terminus of a C-terminal portion of the base editor, wherein at least one of the first nucleotide sequence and second nucleotide sequence is operably linked to a first promoter. In certain embodiments, at least one of the first nucleotide sequence and second nucleotide sequence comprises (e.g., at its 3′ end) a gRNA nucleic acid segment encoding a guide RNA operably linked to a second promoter. In some embodiments, the direction of transcription of the guide RNA nucleic acid segment is reversed relative to the direction of transcription of the at least one nucleotide sequence. In some embodiments, the gRNA is provided on a separate vector or nucleotide sequence. In some embodiments, the N-terminal portion of the base editor comprises a portion of any one of SEQ ID NOs: 350-388 that corresponds to amino acids 1-573 of the napDNAbp domain of the base editor, and the C-terminal portion of the base editor comprises a portion of any one of SEQ ID NOs: 350-388 that corresponds to amino acids 574-1368 of the napDNAbp domain of the base editor.

In other embodiments, the base editors may be delivered by ribonucleoprotein complexes. In this aspect, the base editors may be delivered by non-viral delivery strategies involving delivery of a base editor complexed with a gRNA (i.e., a BE ribonucleoprotein complex) by various methods, including electroporation and lipid nanoparticles. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Pharmaceutical Compositions

Other aspects of the present disclosure relate to pharmaceutical compositions comprising any of the adenosine deaminases, base editors, or the base editor-gRNA complexes described herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

In some embodiments, any of the base editors, gRNAs, and/or complexes described herein are provided as part of a pharmaceutical composition. In some embodiments, the pharmaceutical composition comprises any of the base editors described herein. In some embodiments, the pharmaceutical composition comprises any of the complexes provided herein. In some embodiments pharmaceutical composition comprises a gRNA, a base editor, and a pharmaceutically acceptable excipient. Pharmaceutical compositions may optionally comprise one or more additional therapeutically active substances.

In some embodiments, compositions provided herein are formulated for delivery to a subject, for example, to a human subject, in order to effect a targeted genomic modification within the subject. In some embodiments, cells are obtained from the subject and contacted with a any of the pharmaceutical compositions provided herein. In some embodiments, cells removed from a subject and contacted ex vivo with a pharmaceutical composition are re-introduced into the subject, optionally after the desired genomic modification has been effected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; 7,163,824, 9,526,784; 9,737,604; and U.S. Patent Publication Nos. 2018/0127780, published May 10, 2018, and 2018/0236081, published Aug. 23, 2018, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated in its entirety herein by reference) discloses various excipients used in formulating pharmaceutical compositions and known techniques for the preparation thereof. See also PCT application PCT/US2010/055131, filed Nov. 2, 2010 (Publication No. WO 2011/053982, published May 5, 2011), incorporated in its entirety herein by reference, for additional suitable methods, reagents, excipients and solvents for producing pharmaceutical compositions comprising a nuclease. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is administered to a subject along with a standard of care treatment for visual loss or hearing loss. Certain visual loss and hearing loss therapies are known in the art.

In some embodiments, the pharmaceutical compositions described herein can be combined with immunomodulatory treatments such as, e.g., inhibitors of a checkpoint molecule (e.g., PD-1, PD-L1, PD-L2, CTLA-4, LAGS, TIM-3, VISTA, TIGIT, CSF1R, CD112R (PVRIG), CD155 (PVR), B7-H2, B7-H4, BTLA, or A2aR). In some embodiments, the pharmaceutical compositions described herein are administered sequentially (in any order) or substantially simultaneously with an anti-PD-1 or anti-PD-L1 antibody, such as nivolumab, pembrolizumab, avelumab, durvalumab or atezolizumab. Additional examples of immunomodulatory therapies include activators that enhance the activity of stimulatory checkpoint such as CD122 (IL2) agonist, 4-1BB, ICOS ligand, GITR, and OX40. Further useful agents are also referenced in Physician's Desk Reference, 59.sup.th edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

Alternatively or in addition, the treatment of the present disclosure can be combined with a chemotherapeutic agent, for example, pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

The term “combination therapy” as used herein, encompasses administration of the pharmaceutical compositions and one or more other therapies in a sequential manner, that is, wherein each therapeutic agent is administered at a different time, as well as administration of the pharmaceutical compositions and one or more other therapies in a substantially simultaneous manner.

Sequential or substantially simultaneous administration of each agent (therapy) can be affected by any appropriate route including, but not limited to, oral routes, intravenous routes, intramuscular, subcutaneous routes, and direct absorption through mucous membrane tissues. The agents can be administered by the same route or by different routes. For example, a first agent can be administered orally, and a second agent can be administered intravenously.

As used herein, the term “sequential” means, unless otherwise specified, characterized by a regular sequence or order, e.g., if a dosage regimen includes the administration of a first therapeutic agent and a second therapeutic agent, a sequential dosage regimen could include administration of the first therapeutic agent before, simultaneously, substantially simultaneously, or after administration of the second therapeutic agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the agents described herein.

When co-administered with an additional therapeutic agent, suitable therapeutically effective dosages for each agent may be lowered due to the additive action or synergy. The efficacy of the methods described herein (e.g., administration of the pharmaceutical composition alone or as part of a combination therapy) may be assessed by any method known in the art and would be evident to a skilled medical professional. For example, the efficacy of the therapies described herein may be assessed by survival of the subject. In some embodiments, the therapy is assessed based on the safety or toxicity of the therapy in the subject, for example, by the overall health of the subject and/or the presence of adverse events or severe adverse events.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In one embodiment, the pharmaceutical composition is formulated for delivery to a subject via the intratumoral route.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

In some embodiments, any of the disclosed pharmaceutical compositions are administered subretinally. In particular embodiments, any of the disclosed pharmaceutical compositions are administered subretinally to the eye of a subject. In some embodiments, any of the disclosed pharmaceutical compositions are administered to the inner ear of a subject. In particular embodiments, the disclosed pharmaceutical compositions are formulated for delivery to a retina or an inner ear of the subject by injection.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. Optionally, the kit may comprise a further therapy, for example, (c) a contain containing an immune checkpoint inhibitor (e.g., anti-PD-1 antibody). The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Kits, Cells, and Uses

This disclosure provides kits comprising a nucleic acid construct comprising nucleotide sequences encoding the base editors, gRNAs, and/or complexes described herein. Some embodiments of this disclosure provide kits comprising a nucleic acid construct comprising a nucleotide sequence encoding an deaminase-napDNAbp base editor capable of deaminating an adenosine in a nucleic acid molecule. In some embodiments, the nucleotide sequence encodes any of the ABEs or CBEs provided herein. In some embodiments, the nucleotide sequence encodes any of the complexes of ABEs or CBEs and gRNA sequences provided herein. In some embodiments, the nucleotide sequence comprises a heterologous promoter that drives expression of the ABE. The nucleotide sequence may further comprise a heterologous promoter that drives expression of the gRNA, or a heterologous promoter that drives expression of the base editor and the gRNA.

In some embodiments, the kit further comprises an expression construct encoding a guide nucleic acid backbone, e.g., a guide RNA backbone, wherein the construct comprises a cloning site positioned to allow the cloning of a nucleic acid sequence identical or complementary to a target sequence into the guide nucleic acid, e.g., guide RNA backbone.

The disclosure further provides kits comprising a base editor as provided herein, a gRNA having complementarity to a target sequence, and one or more of the following: cofactor proteins, buffers, media, and target cells (e.g., human cells). Kits may comprise combinations of several or all of the aforementioned components.

Some embodiments of this disclosure provide kits comprising a nucleic acid construct, comprising (a) a nucleotide sequence encoding a napDNAbp (e.g., a Cas9 domain) fused to an deaminase domain; and (b) a heterologous promoter that drives expression of the sequence of (a). In some embodiments, the kit further comprises an expression construct encoding a guide nucleic acid backbone, e.g., a guide RNA backbone, wherein the construct comprises a cloning site positioned to allow the cloning of a nucleic acid sequence identical or complementary to a target sequence into the guide nucleic acid, e.g., guide RNA backbone.

Some embodiments of this disclosure provide cells comprising any of the base editors or complexes provided herein. In some embodiments, the cells comprise nucleotide constructs that encodes any of the base editors described herein. In some embodiments, the cells comprise any of the nucleotides or vectors provided herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Pancl, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML Ti, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalcic7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassus, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some aspects, the present disclosure provides uses of any one of the base editors described herein and a guide RNA targeting this base editor to a target in the USH2A gene in the manufacture of a medicament. In some aspects, uses of any one of the base editors and guide RNAs described herein are provided in the manufacture of a kit for base editing, wherein the base editing comprises contacting the nucleic acid molecule with the base editor and guide RNA under conditions suitable for the substitution of the adenine (A) of a A:T nucleobase pair in the target with a guanine (G), or for the substitution of the cytosine (C) of a C:T nucleobase pair in the target with a thymine (T). In some embodiments, the step of contacting induces separation of the double-stranded DNA at a target region. In some embodiments, the step of contacting thereby comprises the nicking of one strand of the double-stranded DNA, wherein the one strand comprises an unmutated strand that comprises the T of the target A:T nucleobase pair, in adenine base editing. In some embodiments, the nicked strand comprises an unmutated strand that comprises the G of the target C:G nucleobase pair, in cytidine base editing.

For example, in some embodiments of the disclosed adenine base editing methods, the strand that includes the adenine (A) of the target T:A nucleobase pair opposite the strand containing the target thymine (T) that is being excised. This nicking result serves to direct mismatch repair machinery to the non-edited strand, ensuring that the modified nucleotide is not interpreted as a lesion by the cell's machinery. This nick may be created by the use of an nCas9.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in the inner ear of a subject. In some embodiments, the step of contacting is performed in the retina of a subject. In some embodiments, the step of contacting is performed in a cell, such as a human or non-human animal cell.

The present disclosure also provides uses of any one of the base editors or any one of the complexes of base editors and guide RNAs described herein as a medicament. The present disclosure also provides uses of the described pharmaceutical compositions or cells comprising, and vectors or rAAV particles encoding, any of the disclosed base editors or complexes herein as a medicament. In some embodiments, the medicament is for treatment of Usher syndrome (e.g., Usher syndrome, type 2A). In particular embodiments, the medicament is for treatment of autosomal recessive retinitis pigmentosa, vision loss, or hearing loss.

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### EXAMPLES

Data provided in the below examples describe the use of adenine base editors that are capable of catalyzing hydrolytic deamination of adenosine (forming inosine, which base pairs like guanine (G)) to treat Usher syndrome and retinitis pigmentosa by editing USH2A. Base editors comprising an adenosine deaminase (e.g., adenine base editors) have been described previously, for example, in PCT/US2017/045381 (published as WO 2018/027078); PCT/US2018/056146 (published as WO 2019/079347); PCT/2019/033848 (published as WO/2019/226953), and Gaudelli, N. M. et al. “Programmable base editing of A⋅T to G⋅C in genomic DNA without DNA cleavage.” Nature 551, 464-471 (2017). The first deoxyadenosine deaminases were evolved to accept DNA substrates and deaminate deoxyadenosine (dA) to deoxyinosine. As one example, evolution experiments were performed using the adenosine deaminase acting on tRNA (ADAT) from Escherichia coli (TadA, for tRNA adenosine deaminase A), to engineer adenosine deaminases that act on DNA. Briefly, ecTadA was covalently fused to a dCas9 domain, and libraries of this fusion were assembled containing mutations in the deaminase portion of the construct. Adenine base editors (ABEs) mediate the programmable conversion of A⋅T to G⋅C allows for base pair editing.

Base editing is a form of genome editing that enables the direct, irreversible conversion of one base pair to another at a target genomic locus without requiring double-stranded DNA breaks (DSBs), homology-directed repair (HDR) processes, or donor DNA templates. Compared with standard genome editing methods to introduce point mutations, base editing can proceed more efficiently, and with far fewer undesired products such as stochastic insertions or deletions (indels) or translocations.

Base editing capabilities have expanded through the development of base editors with different protospacer-adjacent motif (PAM) compatibilities, narrowed editing windows, enhanced DNA specificity, and small-molecule dependence. Fourth-generation base editors (BE4 and BE4-Gam) further improve C⋅G to T⋅A editing efficiency and product purity. Seventh-generation ABEs such as ABE7.10 convert A⋅T to G⋅C at a wide range of target genomic loci in human cells with a high efficiency, and with a very high degree of product purity (>99%), exceeding the typical performance characteristics of earlier generation base editors, e.g., BE3.

### Example 1

### Base Editing of USH2A In Vitro

First, candidates of USH2A mutation that could be converted by the disclosed adenine and cytidine base editors in silico were selected. To identify potential BE-treatable USH2A mutations, all previously-reported USH2A mutations were compiled using information from the LOVD and HMGD databases.^{1,2} This list was narrowed to those that were pathogenic or likely pathogenic, and to those that have been reported more than once in the LOVD database. Of the 1,260 mutations in USH2A extracted from these databases, 389 were both pathogenic and also were reported more than once (FIG. 2). Then, the sequence context of each mutation was analyzed. Existing base editors are limited to making transition mutations: C to T, T to C, A to G, or G to A, and are also limited by the presence of a PAM sequence within a certain editing window location. The editing window was set at from position 2 to 13 (a distance of 8 to 18 bases from the 3′ end of the PAM sequence) to select variants that could be edited by ABE or CBE variants (FIG. 2). The PAM variants included were NGG (spCas9WT), NGA (spCas9VQR), NG (spCas9-NG), NNGRRT (saCas9WT), and NNNRRT (saCas9KKH). 142 mutations met these constraints. 35 of these mutations were manually selected for editing experiments in human HEK293 cells. Many of these mutations are those that had been more frequently reported in humans. The editing efficiencies of base editors for these candidates were tested in HEK293 cells. It was found that many mutations of USH2A could be edited, with restoration of codon, by a cytidine base editor converting C (or G) to T (or A) (Table 4) or an adenine base editor converting A (or T) to G (or C) (Table 5).

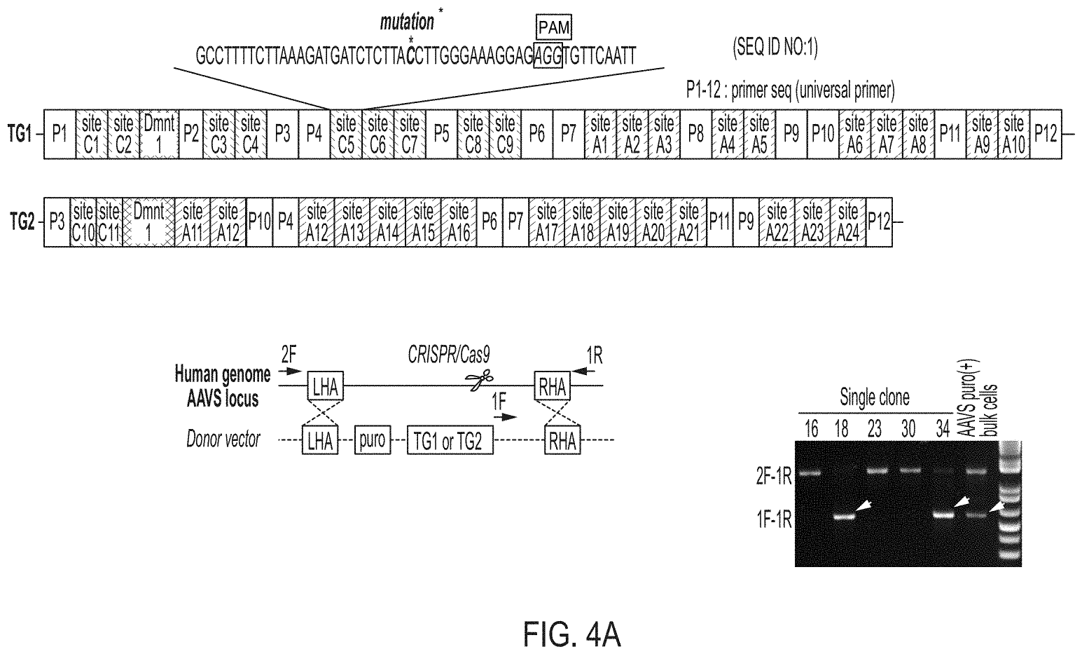

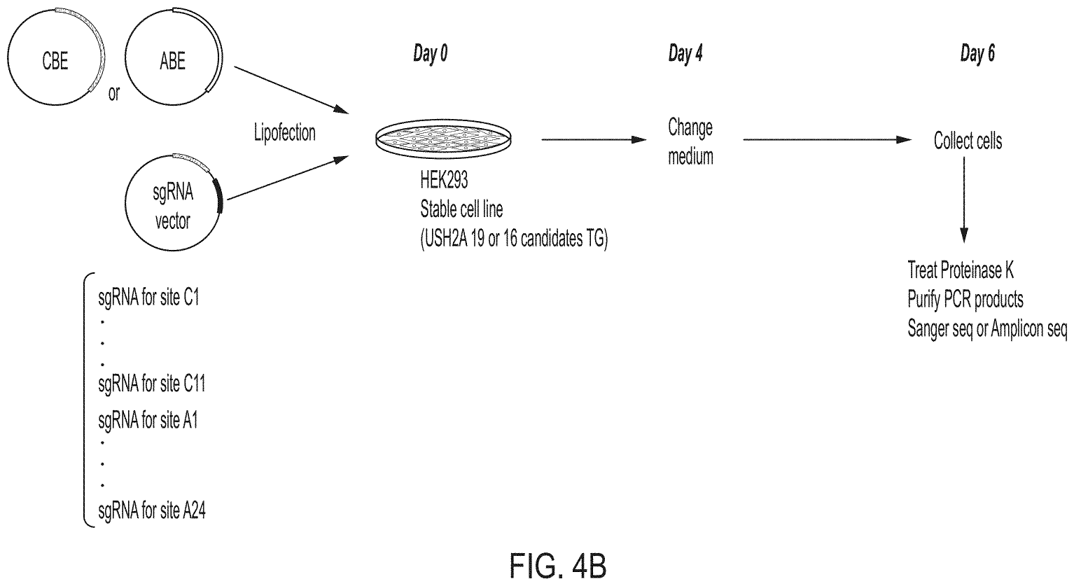

Cell lines containing the mutant sequences that were selected as base editing targets are not readily available. USH2A mutations suitable for the disclosed base editors were selected in silico, and HEK293 stable cell lines which introduced 50 nucleobases that include the candidate mutations of the USH2A gene into the AAVS1 locus were made in order to confirm the efficacy of base editor. these candidate mutations in the USH2A gene are not present in a wildtype genome, and collections of patient-derived cell lines containing these mutations are not available. That is, stable HEK293 cells that express any one (but only one) of the candidate mutations cannot feasibly be prepared. Therefore, a stable cell line evaluation system was generated using an USH2A transgene containing a tandem array of target sites in order to efficiently test to editing efficacy on 35 targets. To introduce multiple mutant sequences into cells efficiently, two synthetic constructs were designed that are composed of tandem arrays of 51bp fragments, each containing a target mutation and adjacent sequence context. Universal primers were incorporated into the sequence to facilitate amplification of the targeted mutations sites by PCR (FIG. 4A). The two transgene constructs were named “TG1” and “TG2”, and altogether contained 36 mutation target sites to be tested, named “Al” through “A24”, “C1” through “C11”, and “Dnmtl” (FIGS. 3A and 3B). TG1 was used in conjuncation with editing of loci C1-C10 and Al-A10. TG1 was used in conjuncation with editing of loci A11-A24. (Under this naming convention, an USH2A target site labeled “A1” refers the first USH2A mutation locus intended to be edited by an A-base editor, and so on (see FIGS. 3A and 3B, “Exp#” column).)

AAVS1 is a safe harbor locus in the human genome, reported as a region that is not affected by gene knock-ins. In an AAVS1 knock-in system, CRISPR/Cas9-mediated homologous recombination causes knock-in of a transgene of choice into the AAVS/locus. Base editor vectors (CBE3.9, ABE7.10, and ABE8) and sgRNA vectors corresponding to each target were transfected into the AAVS/safe harbor knock-in USH2A mutation sequences or base editor vectors, sgRNA vectors and vectors including USH2A mutation sequences were transfected in HEK293 cells. Amplicons including mutations that had been amplified by PCR were read by Sanger sequencing or Amplicon sequencing to quantify editing efficacy.

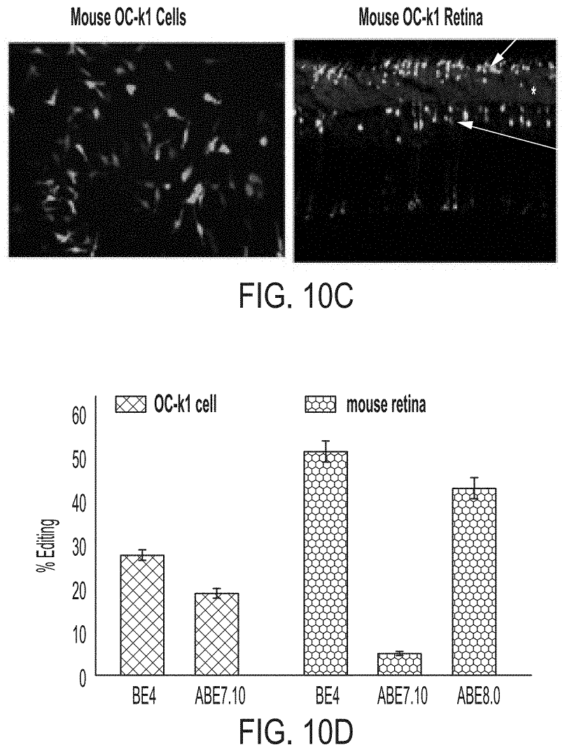

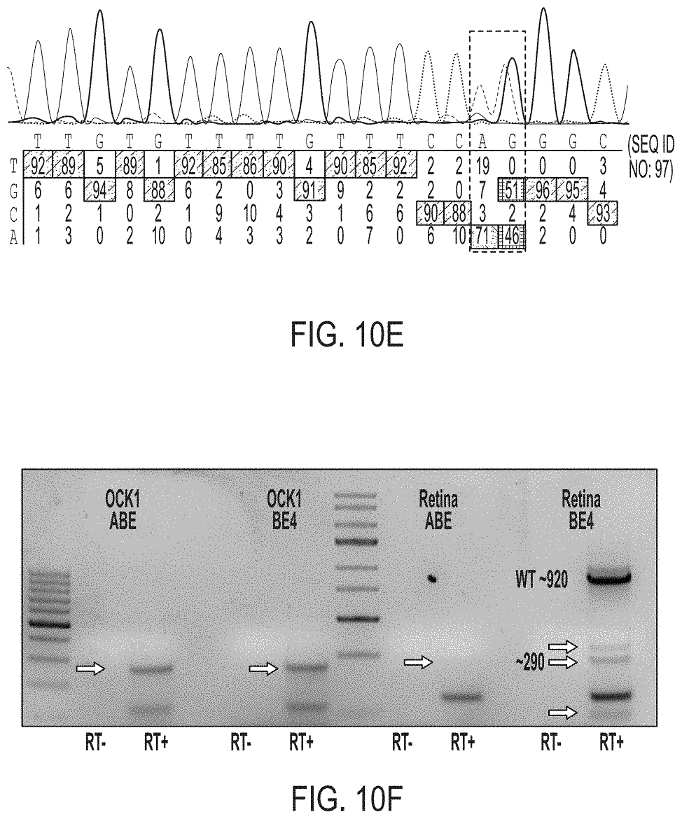

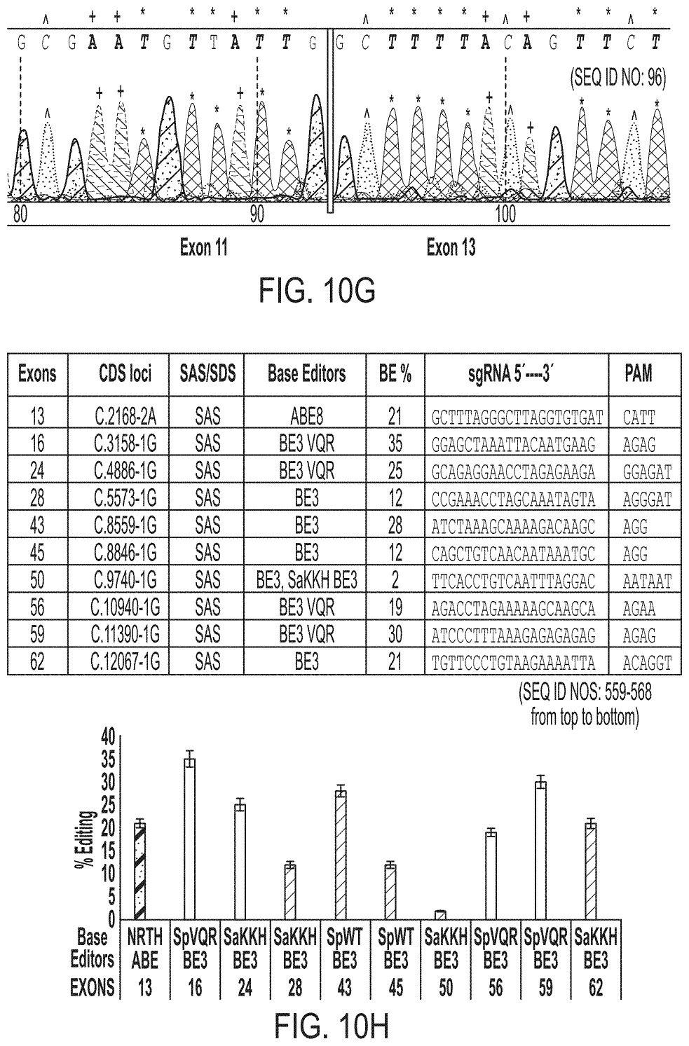

A proof-of-concept study is performed to determine if base editors can introduce skipping of exons by mutating splice sites of the Ush2a gene in mouse organ of Corti (OC-k1) cells and mouse retina. Two sgRNAs are designed to target the SAS (AG) upstream the exon 12 in mouse Ush2a gene. Guide BE4 is used with the BE4-SpCas9-VRQR CBE editor to mutate an AG to AA; and guide ABE7.10 and ABE8e was used with ABE-xCas9-NG editor to convert AG to GG (FIG. 10B). The BE-Cas9 plasmid and the U6-sgRNA-CAG-EGFP were co-transfected into OC-k1 cells via lipofectamine 3000 or into mouse retina via sub-retinal injection and in vivo electroporation (FIG. 10E). Experiments are performed in triplicate. The EGFP-positive cells are isolated using fluorescence-activated cell sorting (FACS) and subjected to DNA and RNA. Immunocytochemistry may be subsequently performed: cells are stained overnight with Ush2a (1:4000) and acetylated-alpha tubulin (1:1000) antibodies, followed by labeling with fluorescent conjugated secondary antibodies.

### High Efficiency Base Editing of Splice Sites in Cells and Mouse Retina

Proof-of-concept studies were performed to determine if base editors (BE) can introduce skipping of exons by mutating splice sites of the Ush2a gene in mouse organ of Corti (OC-kl) cells and in vivo into mouse retina. Two sgRNAs were designed to target the SAS (AG) upstream the exon 12 in mouse Ush2a gene (FIG. 10A).

A guide sequence compatible with BE4 was used with the BE4-SpCas9-VRQR CBE editor to convert an AG to AA; and a guide sequence compatible with ABE7.10 and ABE8e was used with ABE-xCas9-NG editor to convert an AG to GG (FIG. 10B). The BE-Cas9 plasmid and the U6-sgRNA-CAG-EGFP were co-transfected into OC-k1 cells via Lipofectamine 3000®. And the BE-Cas9 plasmid and the U6-sgRNA-CAG-EGFP were co-transfected into mouse retina via subretinal injection and in vivo electroporation (FIG. 10C). Three experiments for each base editor were performed. The EGFP-positive cells were isolated using fluorescence-activated cell sorting (FACS) and subjected to DNA and RNA.

Genomic DNA was extracted and a ˜265 bp portion of Ush2a centered on the target splice site was amplified by PCR. The amplicon from each sample was sequenced using Sanger sequencing and next-generation sequencing (NGS). The base editing rate was determined based on the NGS reads that were processed through CRISPResso, a public software that provides analyses of genome editing outcomes from deep sequencing data. See Clement et al., Nat Biotechnol. 2019 Mar; 37(3):224-226, herein incorporated by reference.

EditR analysis was used to quantify the editing efficiency. The editing rate of BE4 at the target base “C4” position was 29% and 52% in OC-k1 cells and retina, respectively. For ABE7.10 editing at “A5” position, the editing rate is 20% and 6% in OC-k1 cells and retina, respectively. ABE8.0 in mouse retina resulted in a 42% editing rate, 6-fold more efficient than ABE7.10 (FIG. 10D). Bystander editing of BE4 at other cytosine positions was at minimum rate (2%) if these edits are all considered (FIG. 10E).

To determine if the sequence alteration at the SAS of Ush2a exon 12 could lead to the skipping of the exon, RNA was extracted from EGFP-positive and negative OC-k1, retinal cells were dissociated, and RT-PCR was performed using primers flanking the adjacent upstream and downstream exon 11 and 13. The expected size of the RT-PCR product in a Southern blot would be 920 bp if exon 12 is included, or 290 bp if exon 12 is deleted. A 278 bp band was detected for both BE4-sgRNA1- and ABE-sgRNA2-treated OC-k1 and in mouse retina (FIG. 10F). This band, detected in all editing cells, matches the expected size if exon 12 is skipped from the transcripts.

Sanger sequencing of the 278 bp band revealed that exons 11 and 13 were spliced correctly (FIG. 10G). Notably, along with the 278 bp band, several smaller sized bands were detected in BE4-SpCas9-WT treated cells, probably resulting from alternative splicing events. Thorough analysis of the transcript events after base editing are being conducted to better understand the consequences of BE-mediated exon skipping. Overall, this proof-of-concept study demonstrates a highly efficient base editing rate at the splice site with either CBE or ABE editors both in vitro and in vivo, consistent with published values for base editing. More promisingly, it demonstrated that the mutated splice site induced by base editing could lead to an exon-skipping event in the cells and retina.

### Methods

### In Silico Selection of Potentially Editable Human USH2A Mutations

The USH2A mutation information was downloaded from LOVD (https://databases.lovd.nl/shared/genes/USH2A) and HMGD (http://www.hgmd.cf.ac.uk/ac/index.php). Likely pathogenic and pathologic mutations in these databases (“pathogenic”, “UV3”, and “UV4” in LOVD; “DM” in HGMD) were selected, with mutations for which only 1 case was reported in LOVD excluded. The editing window was set at from position 2 to 13 (a distance of 8 to 18 bases from the 3 ′ end of the PAM sequence) to select variants that could be edited by an adenine base editor (ABE) or cytosine base editor (CBE). The base editing PAM variants used were NGG, NGA, NG, NNGRRT, and NNNRRT.

### Plasmid Vector Construction

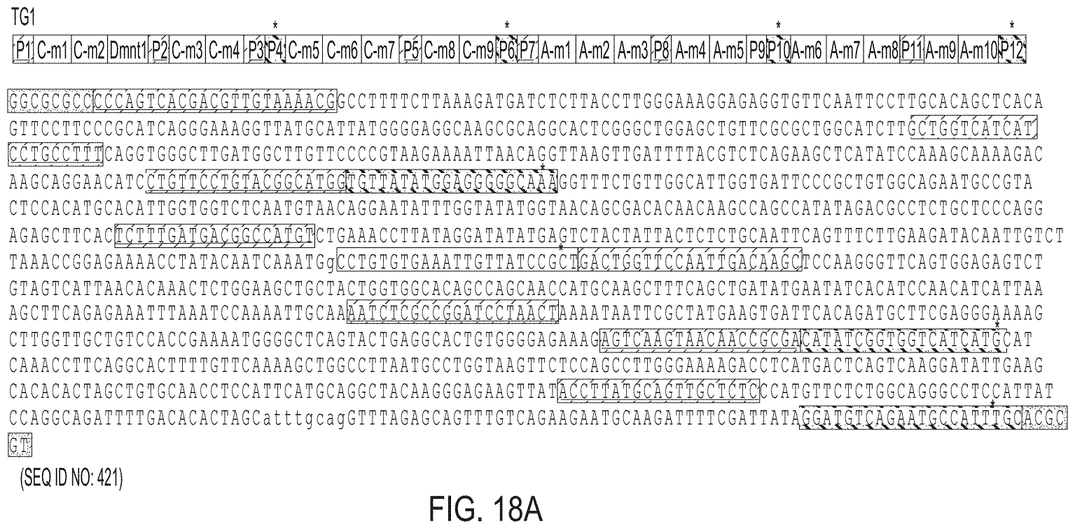

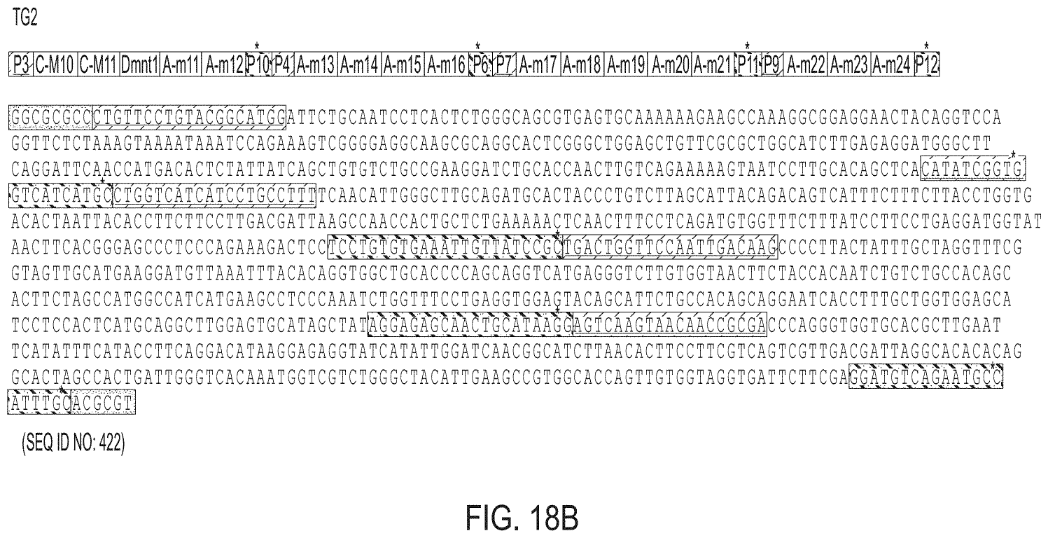

Transgenes including tandem arrays of USH2A mutations (FIGS. 18A-18B) were synthesized by integrated DNA Technologies were inserted into the AsCl/MluI cut site in pAAVS1-puro-DNR plasmid (Origene). The AAVS1 genomic locus was selected for the transgene integration site due availability of existing reagents targeting this locus, rather than any link to adenovirus-associated virus (AAV) biology per se.

Guide RNAs (sgRNAs) were designed to target human USH2A mutation (Table 10). Oligonucleotides were synthesized by Integrated DNA Technologies (Coralville, IA), annealed, and ligated into BsmBI digested BPK1520 (Addgene #65777) for SpCas9 and BPK2660 (Addgene #70709) for SaCas9 in which CAG-EGFP was inserted in HindIII cut site.

The expression plasmid of the cytosine base editor, CBE3.9max3, was used to make C-to-T and G-to-A transitions. PAM specificity variants included SpCas9-WT, SpCas9-VQR, and SaCas9-KKH. The following adenine base editor expression plasmids were used for mutation A-to-G to T-to-C transitions: ABE7.10 SpCas9WT (Addgene #112095), ABE7.10 SpCas9VQR (Addgene #119811), ABE7.10 SpCas9NG (Addgene #124163), ABE7.10 SaKKH (Addgene #119815), ABE8 SpCas9WT (Addgene #138489), ABE8 SpCas9NG (Addgene #138491). For each mutation target site, the appropriate plasmid was used from the lists above, based on the editor type and PAM type.

The full-length wildtype human USH2A plasmid was driven by a CMV promoter in a pUC57 backbone.? The large plasmid size (19.1 kb) makes mutagenesis by PCR inefficient. Human USH2A mutant plasmids were constructed from the wildtype hUSH2A plasmid using Gibson assembly. Briefly, the wt USH2A backbone was digested with BlpI (Al), SacII (A7), PsHAI and NotI (A12), PshAI and AvrII (A15) which were the nearest single cutters to each mutation. Two PCR fragments for each mutation were amplified from the WT vector with overhangs and then were assembled together with the cut backbone using Gibson assembly. Primers used for these PCRs were listed in Table 12.

### Establishment of Stable Cell Lines Containing USH2A Mutations

HEK293 cell lines containing USH2A mutant target sequences in AAVS1 were established using the pAAVS1-Puro-DNR plasmid vector (Origene) and the pCas-guide plasmid vector (Origene). The transgenes (TG1, TG2) 51 bp gene fragments, each containing a USH2A mutation, connected in tandem. Universal primers were interspersed among the fragments for PCR amplification of edited sites for quantification. Synthetic oligos (Integrated DNA Technologies) containing the transgene sequences were cloned into the Ascl-MluI cloning site of the pAAVS1-Puro-DNR plasmid vector. 8x 10⁵ HEK293 cells were seeded into 6-well cell culture plates, transfected with 1.6 μg of pAAVS1-puro-DNR and 1.6 μg pCas-guide plasmid vector using 8 μl of Lipofectamine 2000, and cultured in puromycin at final concentration of 1.5 μg/mL for 21 days after transfection. Single cells were expanded into colonies by the limiting dilution method. Insertion of the transgene in the desired AAVS1 location was assayed using PCR with specific primers (AAVS1F and AAVS1R (Table 11) in the donor vector and another primer located in AAVS1 locus.

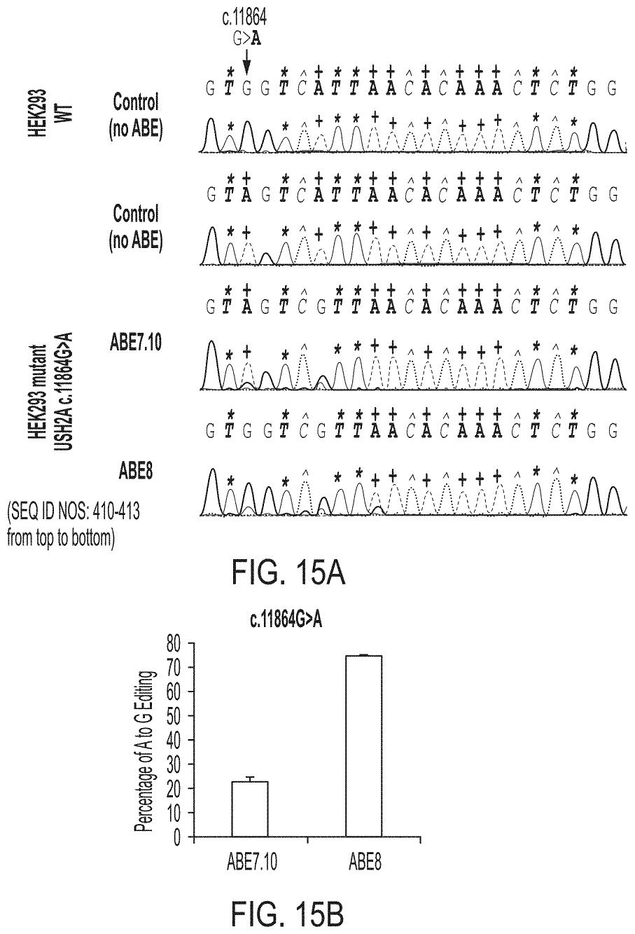

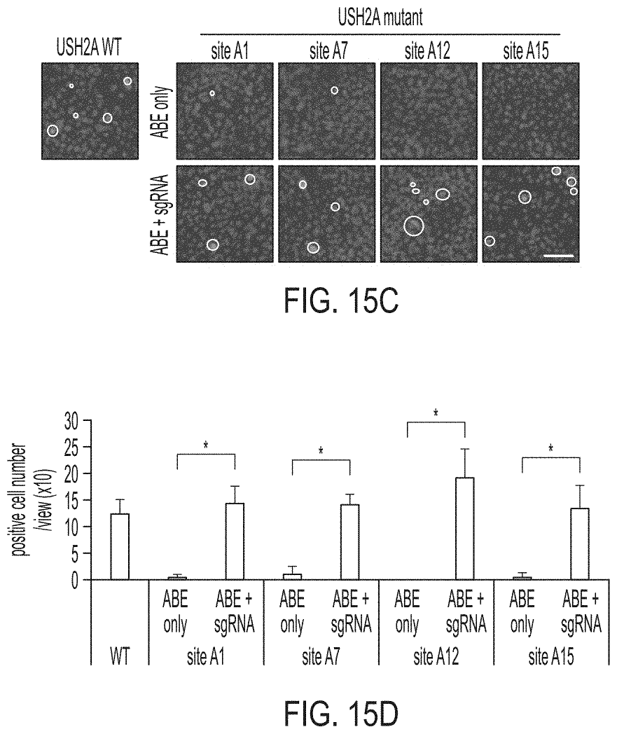

For the establishment of a HEK293 cell line with the site Al mutation (USH2A c.11864G>A) at the endogenous USH2A locus, a donor vector with the c.11864G>A template, an sgRNA-expressing plasmid (with guide spacer AGGGTTCAGTGGAGAGTCTG (SEQ ID NO: 162)) and a Cas9 expression plasmid were transfected into HEK293 cells. Single clones containing the mutation sequence without indels were selected by Sanger sequencing. As a result, a clone in which the mutation was contained in all three (triploid) chromosomes was established (FIG. 15A). Plasmid PX459 (Addgene #48138) containing SpCas9-2A-puro was used for co-transfections with 127 bp ssODN; TCAACAGAACTGAATGAGCACTCGTGGCTTGAGCCCAAGGAGCTGGAAAATCTT GAGGTGGAGCTTCCAGAGTTTGTGTTAATGACTACAGACTCTCCACTGAACCCTT GGAGTTACAGGCTCTGAC (SEQ ID NO: 163) (Integrated DNA Technologies). After single cell cloning, the sequence around mutation site and around the edges of the homology regions was checked by Sanger sequencing and was 100% correct. USH2A cDNA expression constructs restore ciliogenesis in Ush2a null cells

Human USH2A exon 13, 17, 24, 28, 43, 45, 50, 54-55, 56, and 62 deleted (hUSH2A-ΔEx13, ΔEx17, ΔEx24, ΔEx28, ΔEx43, ΔEx45, ΔEx50, ΔEx54-55, ΔEx56 and ΔEx62 plasmids were constructed from the wild type hUSH2A plasmid. For example, to create USH2A-AEx13, the wt hUSH2A backbone was digested with PsHAI and AvrII, which were the nearest single endonuclease sites to exon 13, to remove the sequence between exon 12 and 25. PCR fragments from exons 11-12 (234 bp) and 14-26 (2377 bp) were amplified from the WT vector with overhangs, and then assembled together with the cut backbone using Gibson assembly. A similar approach was used to construct the other plasmids encoding hUSH2A with an excised exon (“deletion plasmids”).

### Base Editing Evaluation by Transfection and Genomic DNA Preparation

1.5×10⁵ HEK293 cells were seeded into 48-well cell culture plates, transfected with 240 ng of base editor vector (BE3.9, ABE7.10 or ABE8) and 80 ng sgRNA plasmid vector, and 0.66 μl of Lipofectamine 2000, and collected day 6 after transfection. To obtain genomic DNA, cells were washed with PBS and lysed with DNA lysis buffer (50 mM Tris HCl pH 7.5, 0.05% SDS, and 5μg/mL proteinase K). The genomic DNA mixture was incubated at 37° C. for 1 h 30min, followed by an 80° C. enzyme denaturation step for 30 min.

### Immunocytochemistry

OC-k1 cells were grown on coverslips and fixed with 1% freshly prepared paraformaldehyde (PFA) for 20 seconds. Samples were blocked in 1% bovine serum albumin and 10% goat serum in PBS (pH 7.4). Separately, in each reaction, each deletion plasmid along with SpCas9-2A-GFP plasmids at 1:1 molar ratio were co-transfected into ˜80% confluence OC-k1 cells in 6-well plate using Lipofectamine 3000 (Thermo Fisher Scientific). The cells were incubated for 48 hours. Cells were stained overnight with Ush2a (1:4000) and Acetylated-alpha tubulin (1:1000) antibodies, followed by labeling with fluorescent conjugated secondary antibodies. Images were taken with a fluorescent microscope (Eclipse Ti, Nikon, Tokyo, Japan). Cilia length was quantified by measure the acetylated tubulin signal using a freehand line tool and a length measurement option in the Image J software (National Institute of Mental Health, Md., USA).

### Example 2

### Editing in Human Cells

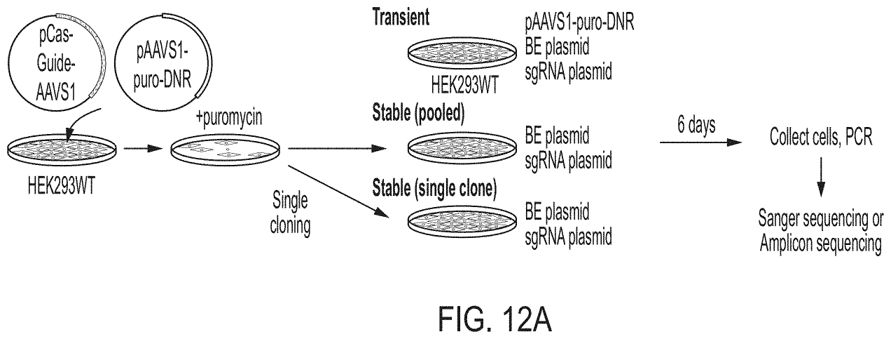

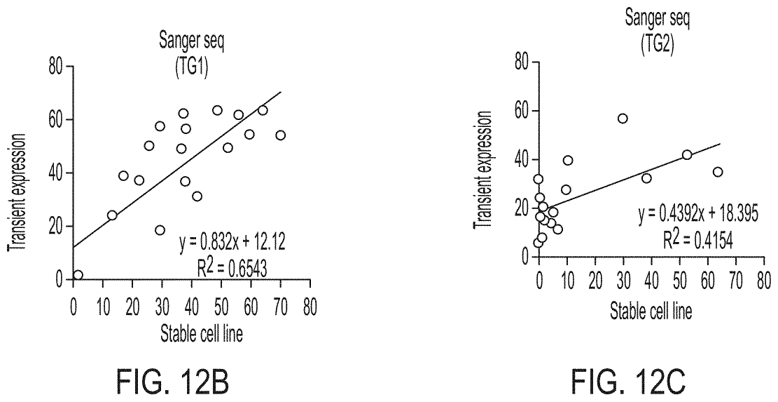

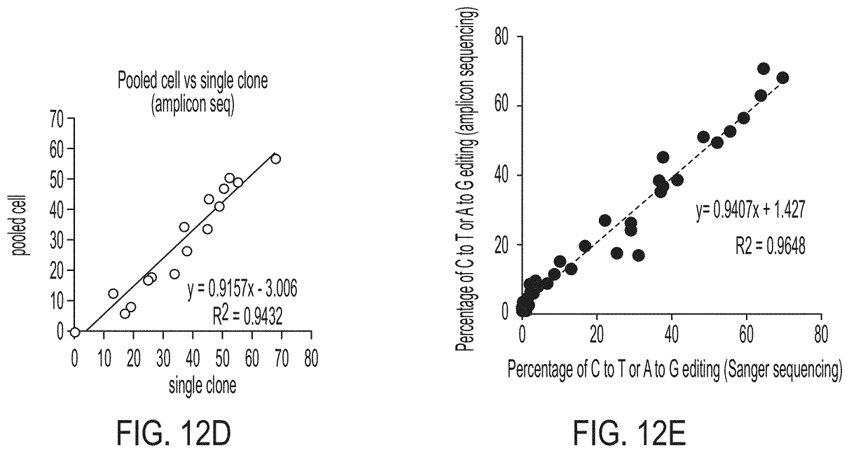

The base editing results between a plasmid-based target introduced by transient transfection versus a stably integrated transgene target incorporated into the AAVS1 genomic locus was compared in HEK293 cells, as shown in FIG. 12A. Stable cell lines with a transgene integrated into the AAVS1 locus were generated using the CRISPR/Cas9 system (FIG. 4B), and integration of the target transgene into the AAVS1 region was confirmed. After transient transfection of base editing reagents (an enzyme plasmid and a separate guide plasmid), each target sequence was amplified by PCR followed by Sanger sequencing to quantify the editing efficiency of the target base, as shown in FIG. 12A. The results showed a high correlation between plasmid-based and transgene-based editing for transgene 1, but not for transgene 2 (FIGS. 12B-12D). In parallel, a

Next, two different methods were evaluated for measuring editing efficiency (target base conversion rate), NGS sequencing versus Sanger sequencing. PCR products amplified using universal primers corresponding to each target were subjected to either NGS amplicon sequencing or Sanger sequencing analysis to compare the editing efficiency of mutation sites. These two data sets showed a high correlation (r²=0.96, FIG. 12E), validating Sanger sequencing as an accurate method for evaluating editing efficacies.

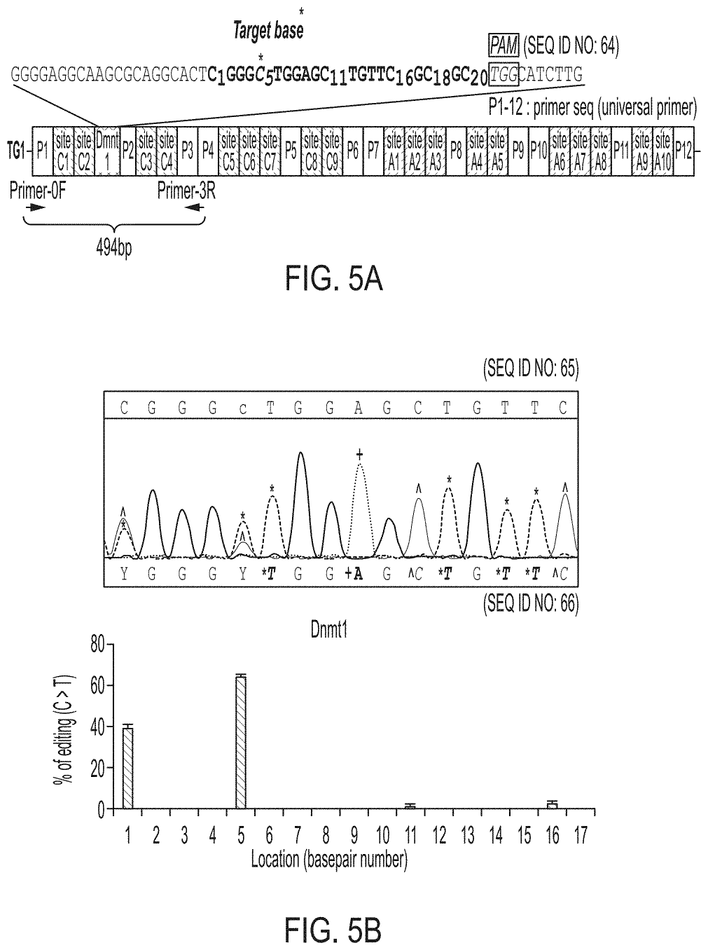

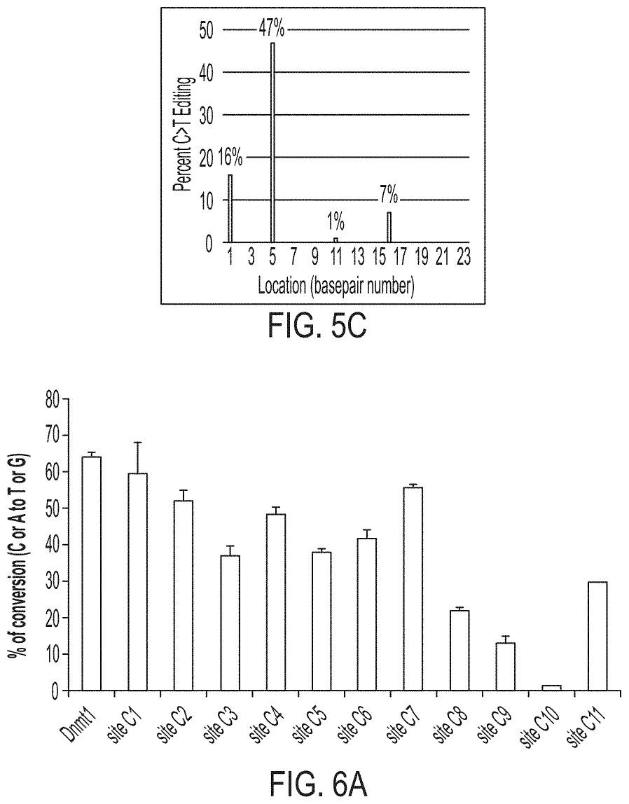

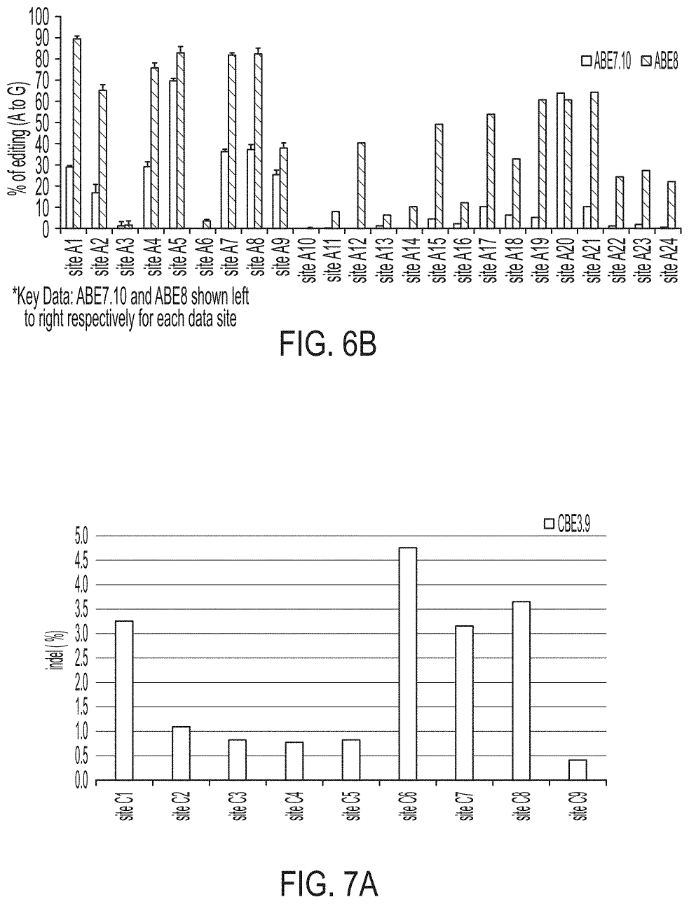

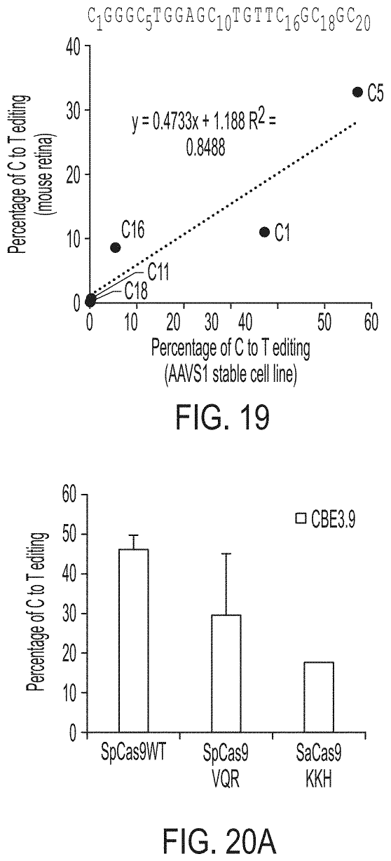

To estimate how these results derived from the HEK293 cells line might compare to editing in the retina in vivo, data from the editing of the genomic transgene in HEK293 cells to the data was compared to editing data in mouse retina in vivo after subretinal AAV delivery of a split-intein formulation of BE3.9, as previously published in Levy et al., Nat Biomed Eng. 2020 Jan; 4(1):97-110. The target cytosine and neighboring (bystander) cytosines in the protospacer sequence of Dnrntl showed a similar editing pattern after subretinal administration as was seen in the transgene experiment (FIG. 19; see also FIGS. 5A-5C). These results demonstrate that the editing efficiency and the editing pattern of base editing in the HEK293 cell line experiments presented in this study likely reflect those in the endogenous genome of photoreceptors in vivo, at least as indicated by results from the Dnrntl test locus. Dnmt1 acts redundantly with Dnmt3a in the mammalian brain and is therefore well-suited for proof-of-concept studies. As previously described, samples were prepared and Sanger sequencing data was received (see Pendse et al., 2020). Mouse retina were administered fifty BE3.9 vectors and sgRNA vectors corresponding to each target at the many targeted sites following Dnrntl knock-in of USH2A mutation sequences. Amplicons including mutations that amplified by PCR were read by Sanger to quantify editing efficacy.

### Evaluation of Base Editing Efficiency on USH2A Target Mutations

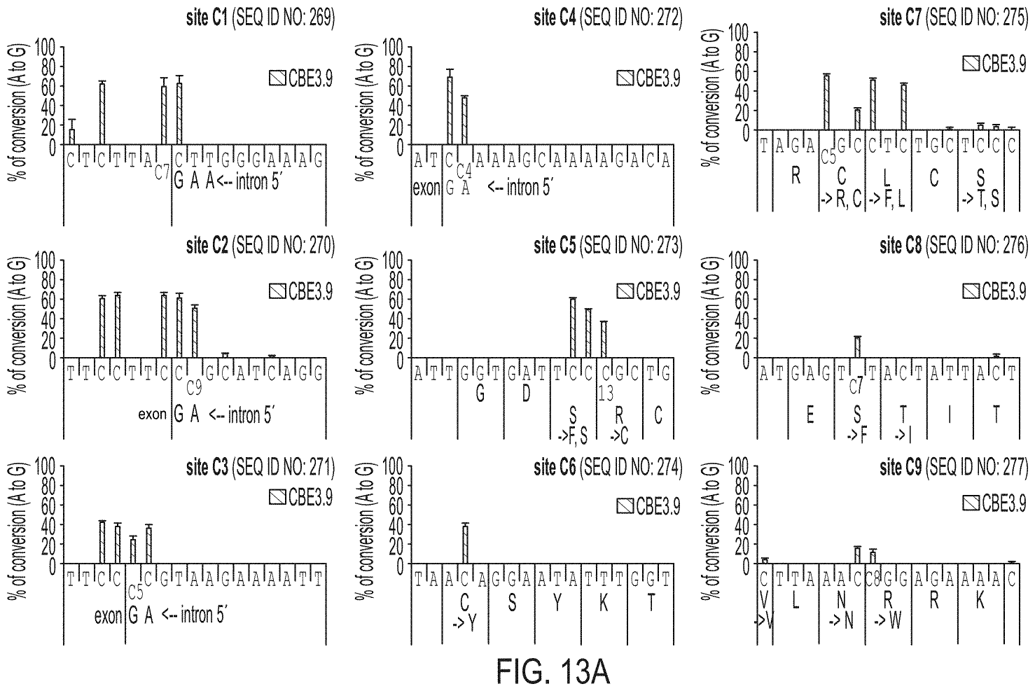

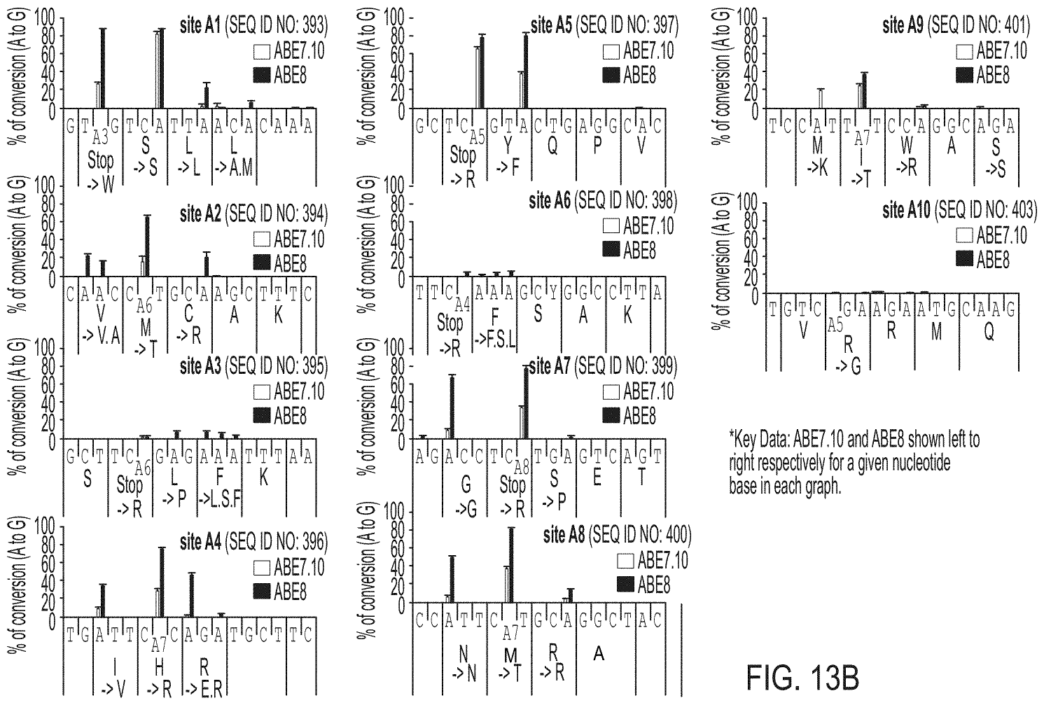

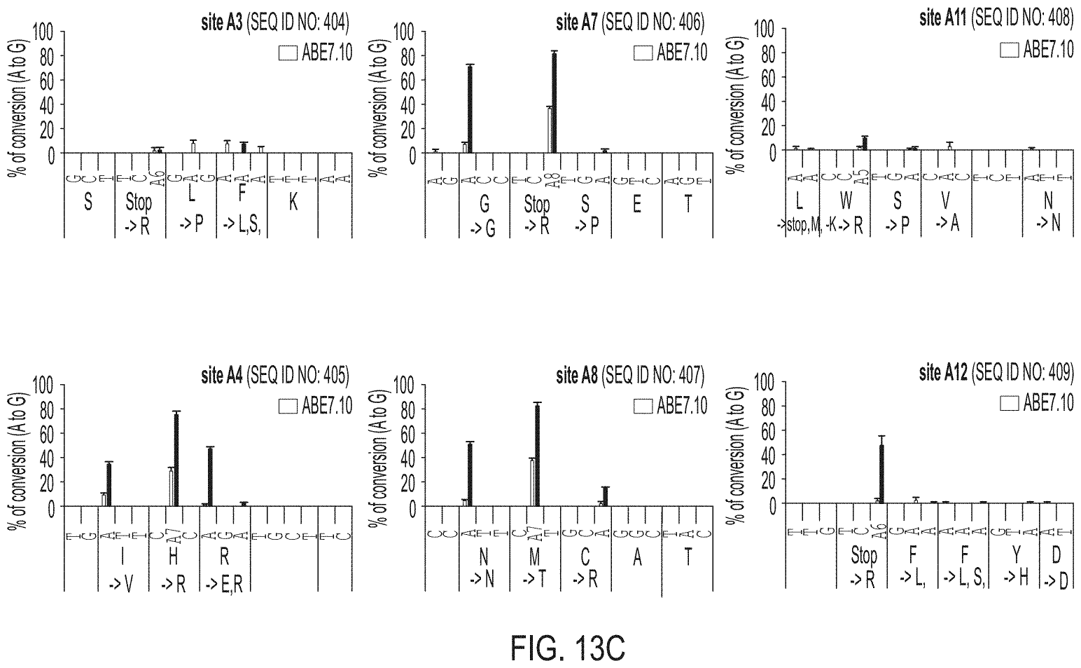

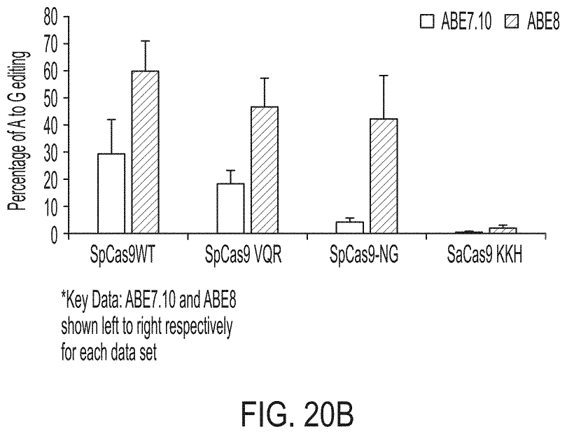

Due to the low correlation of editing efficiency between editing of transiently-transfected plasmids and stably integrated transgenes in one of the two transgenic constructs (TG2, see above), the remaining experiments were performed using a stably integrated transgene. Each transgene is located in the autosomal genome, like the eventual therapeutic targets in photoceptors. In each of the 11 target sites with an intended C-to-T or G-to-A target conversion (sites C1 to C11), editing was evaluated using three different PAM variants of the C-base editor CBE3.9max, using Sanger sequencing as a readout. The results showed CBE editing efficiencies ranging from 59.5±8.4% (site C1) to 1.9±1.9% (site C10) (FIG. 6A). The editing window was rather wide, similar to previous reports4, with editing observed of additional nearby, bystander cytosines in addition to the target cytosine (FIG. 13A). Next, the editing efficacies of four different PAM variants of two A-base editors, ABE 7.10 and ABE 8, were examined on 24 target sites with an A-to-G or G-to-A intended target (FIG. 6B). ABE 7.10 showed editing efficiencies from 69.9 ±0.7 (site A5) to 0% (sites A6, A10, and A14). ABE 8 showed editing efficacies from 89.4±1.3% (site A1) to 0.20±0.35% (site A10). Compared with CBE, ABE tended to show greater variability as to whether a particular editing target showed efficient editing or not. As previously reported5, ABE8 was more efficient and had a wider editing window than ABE 7.10 (FIGS. 13A-13C). Efficiency also varied by the Cas9 PAM variant type used. SpCas9WT tended to have the highest editing efficiency and SaCas9-KKH tended to have the lowest (FIGS. 20A-20B).

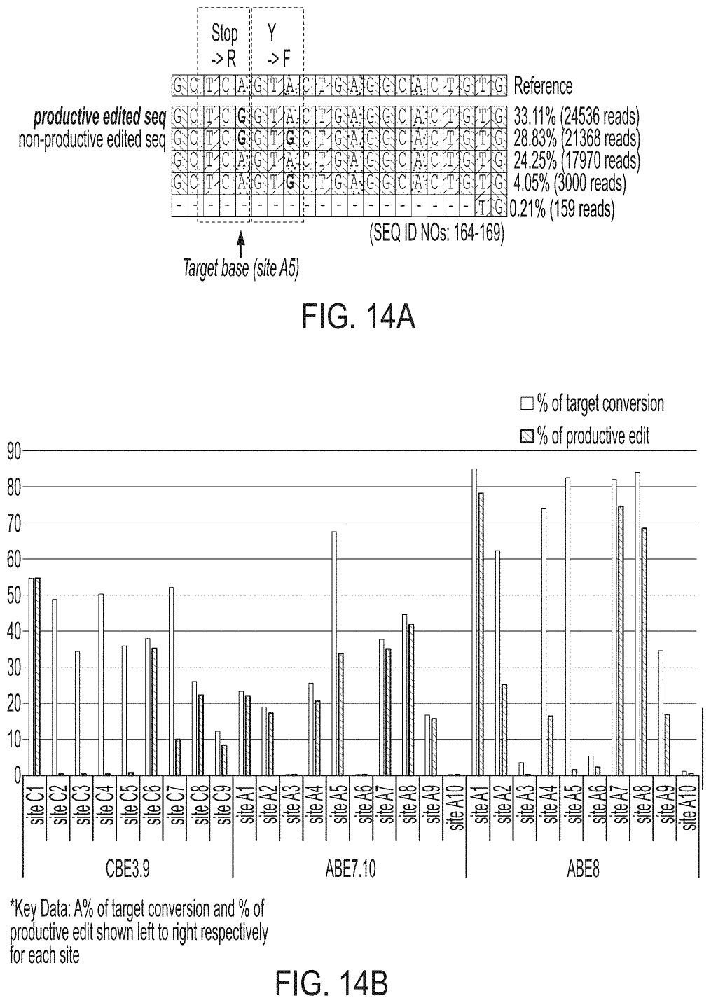

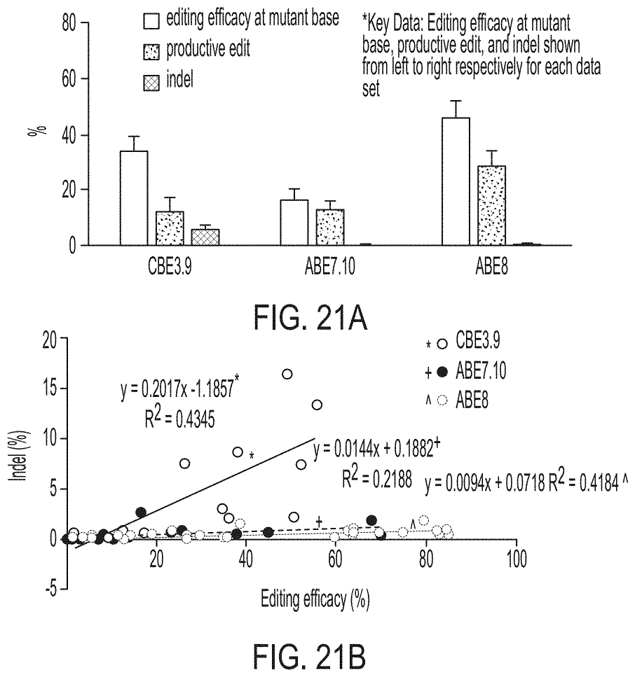

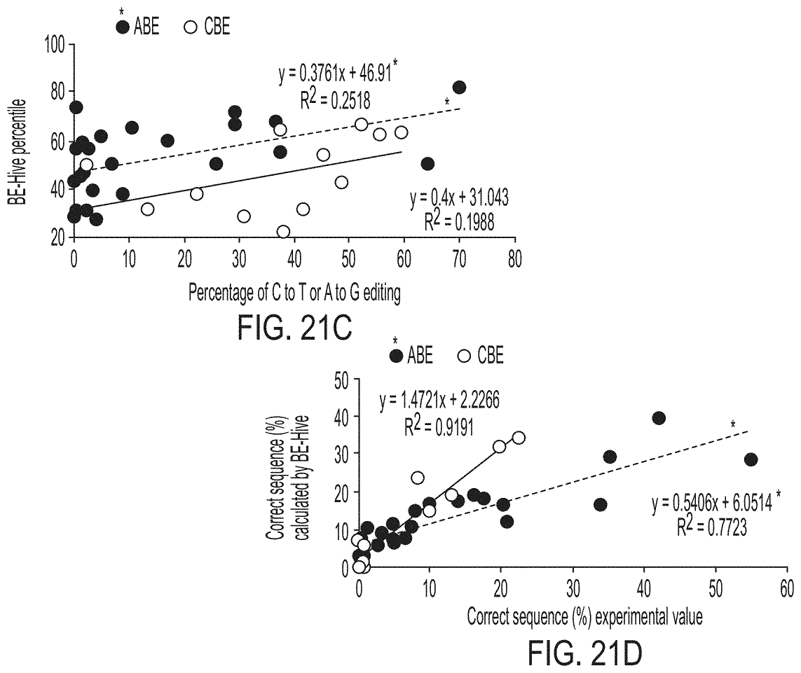

To determine which target sites are most suitable for treatment with BE, with an expected phenotypic recovery at the amino acid level, the effects of bystander editing were evaluated using NGS sequencing and analysis of individual reads. The metric “Productive edits” were defined as reads in which the intended amino acid change was created but no other amino acids were changed (FIG. 14A). This allows for synonymous mutations but does not allow for missense mutations, nonsense mutations, or indels. The fraction of productive edits, compared to total edits, was very low for sites C2, C3, C4, C5, and site A5, whereas the fraction of productive edits was high for sites C1, A1, A7 and A8 (FIG. 14B). The indel ratio in each target was also calculated from the results of NGS amplicon sequencing, since the indel ratio was also an important parameter for evaluation as a therapeutic target. The indel ratio for CBE3.9, ABE 7.10, and ABE 8 editors ranged from 0.54-16.53%, 0-2.74%, and 0.07-1.77%, respectively (FIG. 4b). While the overall trend in editing efficacy among editors was ABE8 >CBE3.9 >ABE7.10, the trend for indel creation was CBE3.9 >ABE7.10 and ABE8 (FIGS. 21C-21D). Thus, ABE8 had the most favorable combination of editing efficiency and low production of indels. Overall, these results demonstrate that multiple USH2A mutations show empirical evidence of high productive edits with low indels, making them candidates for continued evaluation as therapeutic targets by base editing.

As shown in FIGS. 14A and 14B, the off-target editing metric “productive editing” (productive edit %), which is calculated as the mean rate at which the contacting of the target sequence with a base editor results in a product that does not contain an amino acid mutation. Nucleotide site A5, which corresponds to nucleotide position 14803, is shown as an example DNA target. The “reference strand” (SEQ ID NO: 164) represents a wild-type target strand, and the sequences below represent five different base editing outcomes, as read by Amplicon sequencing analysis. The next strand (SEQ ID NO: 165) contains the intended A→G base edit at position 14803. The third strand (SEQ ID NO: 166) contains the intended base edit but also contains an unintended A→G mutation at position 14806, which results in a Y→F amino acid substitution. The fourth strand is wild-type (no edits). Thus, the second and fourth strands are productive, while the third strand represents a non-productive edit. FIG. 14B shows the editing efficiency (% target conversion) and productive editing % at each mutation of USH2A for the base editors BE3.9, ABE 7.10, and ABE8e.

Thus, it is feasible that these USH2A-specific reagents will be similarly active in the retina, like the high efficiency shown explicitly for the DNMT1 target in the mouse retina achieved using a split-intein editor.

### Evaluation of Off-Target Effects of Base Editing for USH2A Mutations

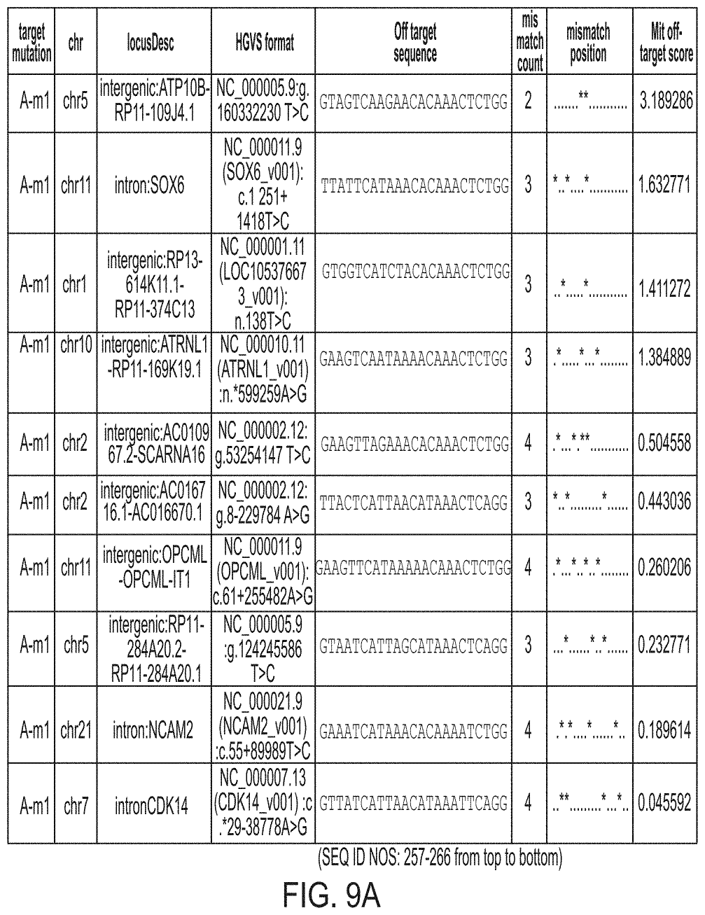

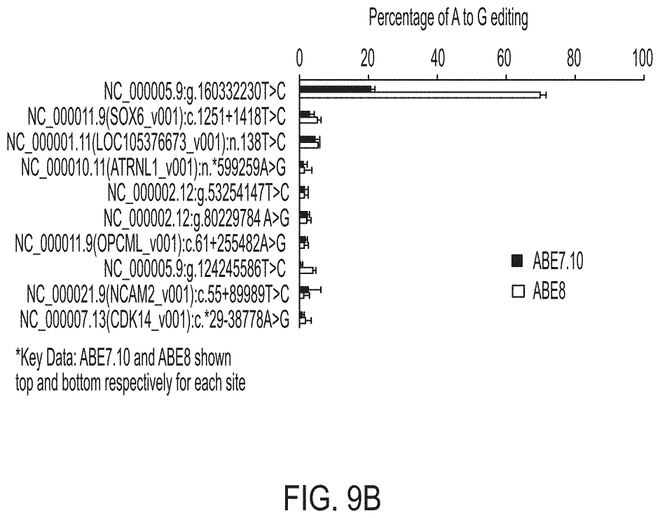

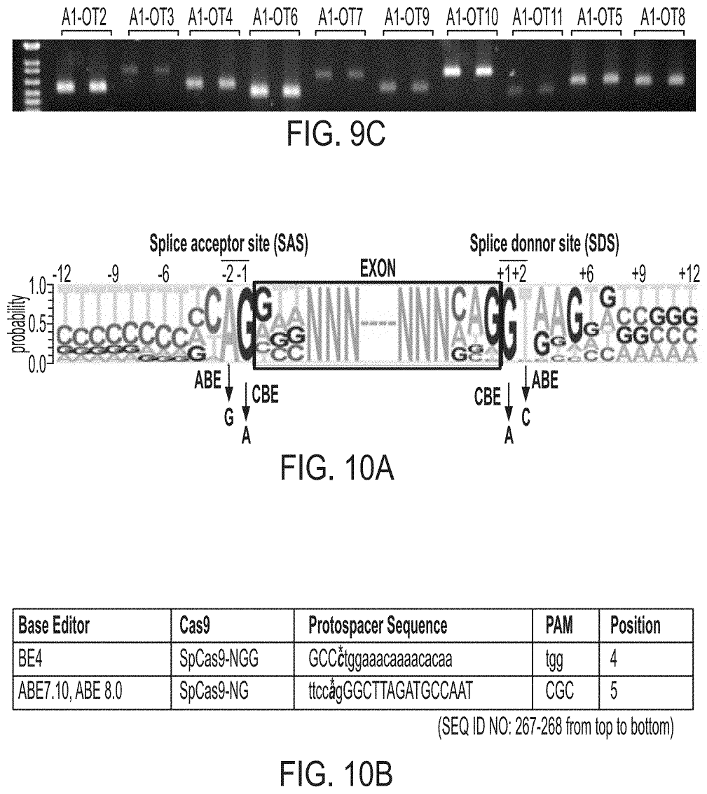

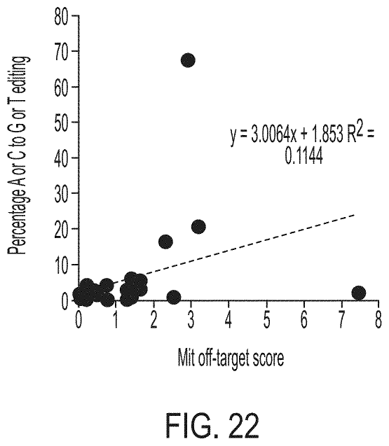

The specificity of base editing depends on how efficiently the sgRNA can recognize other, off-target sites in the genome. Sites C6 and A1 were selected for further evaluation, due to their high base editing efficiency. Off-target effects were examined. The CRISPOR in silico prediction algorithm (Haeussler et al., 2016) was used to identify 10 sequences with a high Mit-off-target score for each target (FIGS. 8A and 9A). After editing, the off-target loci were amplified by PCR and quantified by Sanger sequencing. Off-target editing rates were generally low, but the highest off-target edit with the C6 editing reagents was 19.35%, while the highest off-target editing rate for A1 was 74.50% (FIGS. 8B and 9B). There was little correlation between the Mit-off-target score and editing efficiency (FIG. 22). Because the two loci with high off-target editing efficiencies were located in an intronic region (C6) or in an intergenic region (A1), they are less likely to affect gene expression level. More comprehensive tests for off-target editing⁶ (see Tsai, S. Q. et al. Nat Methods 14, 607-614, (2017) and International Application No. PCT/US2020/624628, filed Nov. 25, 2020, each of which is incorporated by reference herein), as well as whether any off-target edits actually have a deleterious effect on photoreceptor health, can be considered for future work. These results suggest that sites C6 and A1 are promising therapeutic targets for base editing.

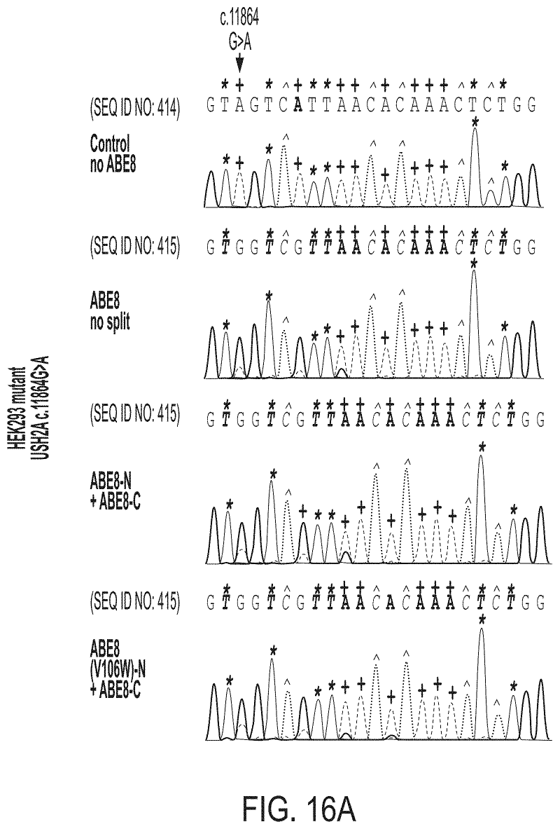

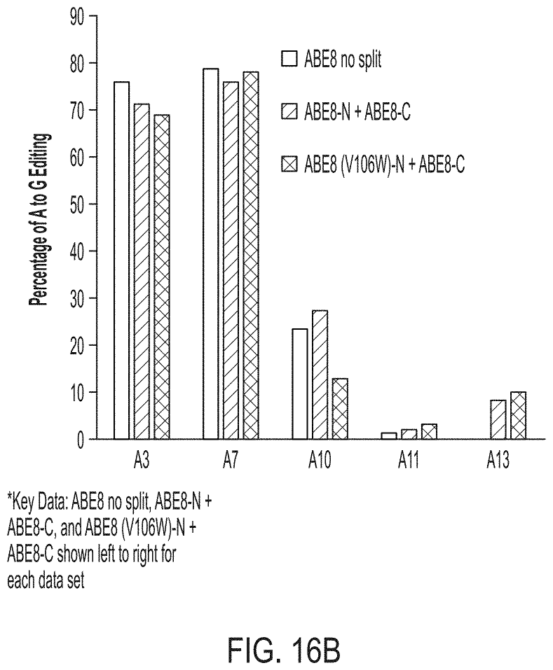

Additionally, it was demonstrated that the base editing reagents described above (at the DNA level) also works to restore protein expression in mutant USH2A cDNAs (FIGS. 16A and 16B). Delivery of base editing reagents to the retina may be accomplished by a number of methods, including viral or nonviral delivery. Adeno-associated virus is a high effective gene therapy delivery vehicle but with limited cargo capacity of ˜4.7kb of DNA. The evaluated split intein-editors are small enough to by carried by AAV viral particle vehicles.

A split intein ABE8 editor and a split intein ABE8(V106W) editor was evaluated, in conjunction with guide RNAs for sites A3, A7, A10, All, or A13 in the USH2A gene to demonstrate that reagents whose DNA lengths are within the 4.7kb limit are capable of successfully editing sites A3 and A7 with high efficiency, and with smaller efficiencies for sites A10, All, A13. An ABE8e editor that was not split into two inteins was also evaluated as a control. Sites A3, A7, A10 indicate where the editing target base is located in the protospacer sequence and correspond to the numbers above the bar graph of FIG. 16B. A3 is the site of c.11864G>A (site A1). A7 and A10 are the bystander editing sites.

Restoration of Full Length USH2A Protein with Base Editing

The experiments above were performed on transgenes integrated into the AAVS1 genomic locus, using a sequence context of 51 bp from each mutation locus. To test whether base editing has the same effect at the endogenous, full-length USH2A genomic locus, a mutant cell line with a homozygous site Al mutation (USH2A c.11864G>A p.Trp3955*) was generated (FIG. 15A). (As used in this Example, the term “homozygous,” means that all three of the triploid chromosomes in the HEK cell genome were mutagenized.) Editing of the A1 site in the endogenous Ush2a locus using ABE7.10 and ABE8 showed an editing efficiency of 23.0±1.6% and 74.8 ±0.5% respectively (FIG. 15B). These results are very similar to those obtained from editing the synthetic construct containing the Al site at the AAVS1 locus, which showed 29.2±0.5% editing with ABE7.10 and 89.4±1.3% editing with ABE8. Using ABE7.10 to edit the same site in a transfected plasmid showed a similar but slightly lower editing efficiency of 18.2±0.2%.

Next, the efficiency of restoration of function of mutant USH2A protein expression following base editing of nonsense mutations at the A1, A7, A12 and A15 sites, which were edited at high efficiency in the above experiments, was examined. A wildtype USH2A expression plasmid ⁷ was modified by mutagenesis to produce the four individual USH2A mutants listed. After co-transfection of USH2A expression vector with ABE and sgRNA plasmids in HEK293 cells, USH2A expression was assessed by immunostaining using an antibody to the C-terminal portion (exon 70-72) of USH2A. No significant immunostaining was detected from any of the four mutant proteins (FIG. 15C). Co-transfection of base editing and sgRNA plasmids fully restored USH2A expression to the same level as wildtype for all four mutants tested (FIGS. 15C and 15D). These results demonstrate that base editing can restore wildtype USH2A protein expression levels for these mutations.

The below tables illustrate the editing efficiencies of exemplary CBE-gRNA combinations in HEK293 (Table 4) and exemplary ABE-gRNA combinations in HEK293 (Table 5) and WERI-RB human retinoblastoma cells (Table 6), in which USH2A mutations were knocked in by transfection.

### Discussion

This study independently demonstrates that ABE8 shows the most favorable characteristics of the base editors tested in this dataset, with high efficiency and low indels. The indel rate was even lower than ABE7.10, even in the context of ABE8's higher editing efficiency. CBE3.9 has a wide editing window, creating “bystander” edits in nearby Cs. While a wide editing window this is a potential disadvantage for mutation reversion, it is an advantage for strategies such as modifying splice sites to promote exon skipping.

The high accuracy of the quantification of Sanger sequencing was surprising, relative to NGS-based amplicon sequencing (r²=0.96, FIG. 12E), which is a gold standard for quantifying the fraction of of bases that are converted. These reliable results for S anger-based quantification were achieved in the context of low background in the Sanger sequencing traces. The possible effects of using noisy sequencing traces were not formally evaluated.

The BE-HIVE AI model provided in PCT/US2021/016924, filed Feb. 5, 2021, which is incorporated herein by reference, offers an opportunity to test how the predictions of the model hold up empirically. In general, an excellent correlation was shown between the model predictions and the empirical editing efficiency for some base editors, but not others (FIGS. 21A-21D). For example, two newly-created editors with the KKH PAM variant were not highly active at any sites (FIG. 20B). These experiments identified that these editors need to be re-engineered before further use and highlight but concrete example of how empirical testing is needed for reagents that were not tested in existing large datasets like BE-HIVE.

In summary, for testing a single target, any of the approaches used (editing of plasmids, editing of a stable transgene fragment, editing of a mutated genomic locus) would provide valid empirical data. Editing of a transfected plasmid editing is the most technically convenient, in cell types that are transfectable. Establishing a single-copy transgene in a particular locus (AAVS) was similar in complexity to creating a single mutant site in the endogenous gene, both using Cas9-based techniques with homology or repair templates, respectively. Faster methods of transgenesis exist, like lentiviral transduction with antibiotic selection or the piggyBac transposase system, though it remains to be seen how the effects of genomic locus and multiple copies might influence editing efficiencies.

For moderate numbers of potential targets to validate, like the 33 targets of Example 2, transgenesis of a target array has an advantage over engineering each endogenous genomic locus separately, because many target fragments can be concatenated into a single transgene. This study using two different transgenes as additional targets were selected, but in practice one large transgene would have been equally technically feasible. The disadvantage of editing concatenated small gene fragments into a transgene is that testing restoration of protein function is not possible, though this can be done as a separate step for top candidates (FIGS. 15A-15D).

For large numbers of potential targets, library approaches become more feasible. Innovative approaches include delivering editors and targets together using lentivirus (BE-Hive) or using recombinase-based methods, such as those disclosed in Matreyek et al., Nucleic Acids Research, Volume 48, Issue 1 (2020), incorporated by reference herein, to create multiple target sites via genome engineering. Synthesizing, cloning, and deconvoluting library results is more complex and expensive than the techniques used in this study (Sanger sequencing and simple amplicon sequencing), but provide orders of magnitude more data.

TABLE 4
Cystosine base editing for reversion of particular mutations in USH2A IN HEK29 3
cells. This table shows guide sequence-base editor combinations that show high editing
efficiency. Top to bottom, the sequences in this table correspond to SEQ ID NOs: 1-11. See
FIGS. 3A and 3B for additional information about the loci described in this table.

| | | | | BE4max | | | | Sequence |
|---|---|---|---|---|---|---|---|---|
| Site | | | Amino Acid | Editing | | | editing | (SEQ ID NOs: |
| # | dnSNP | DNA | Substitution | rate (%) | PAM | Cas9 var | window | 1-11) |
| site | rs786200928 | c.7595- | p.(Lys2532Thrfs*) | 59.47 | NGG | SpCas9WT | C7 | GCCTTTTCTTAAAG |
| C1 | | 2144A > G | | | | | | ATGATCTCTTACCT |
| | | | | | | | | TGGGAAAGGAGAG |
| | | | | | | | | GTGTTCAATT |
| site | rs397518003 | c.1841- | p.(Gly614Aspfs*6) | 52.07 | NGG | SpCas9WT | C9 | CCTTGCACAGCTCA |
| C2 | | 2A > G | | | | | | CAGTTCCTTCCCGC |
| | | | | | | | | ATCAGGGAAAGGT |
| | | | | | | | | TATGCATTAT |
| site | rs397517978 | c.12067- | P-? | 37.12 | NGG | SpCas9WT | C6 | CAGGTGGGCTTGA |
| C3 | | 2A > G | | | | | | TGGCTTGTTCCCCG |
| | | | | | | | | TAAGAAAATTAAC |
| | | | | | | | | AGGTTAAGTTG |
| site | rs397518039 | c.8559- | p.(Tyr2854_Arg289 | 48.60 | NGG | SpCas9WT | C4 | ATTTTACGTCTCAG |
| C4 | | 2A > G | 4del) | | | | | AAGCTCATATCCA |
| | | | | | | | | AAGCAAAAGACAA |
| | | | | | | | | GCAGGAACATC |
| site | rs111033263 | c.9799T > C | p.(Cys3267Arg) | 37.84 | NNGRRT | SaCas9 WT | C13 | GGTTTCTGTTGGCA |
| C5 | | | | | | | | TTGGTGATTCCCGC |
| | | | | | | | | TGTGGCAGAATGC |
| | | | | | | | | CGTACTCCAC |
| site | rs775177930 | c.3368A > G | p.(Tyr1123Cys) | 41.70 | NGG | SpCas9WT | C4 | ATGCACATTGGTG |
| C6 | | | | | | | | GTCTCAATGTAAC |
| | | | | | | | | AGGAATATTTGGT |
| | | | | | | | | ATATGGTAACAG |
| site | rs111033273 | c.1606T > C | p.(Cys536Arg) | 55.60 | NGA | SpCas9 | C5 | CGACACAACAAGC |
| C7 | | | | | | VRQR | | CAGCCATATAGAC |
| | | | | | | | | GCCTCTGCTCCCAG |
| | | | | | | | | GAGAGCTTCAC |
| site | — | c.4325T > C | p.(Phe1442Ser) | 22.14 | NNNRRT | SaCas9 | C7 | CTGAAACCTTATA |
| C7 | | | | | | KKH | | GGATATATGAGTC |
| | | | | | | | | TACTATTACTCTCT |
| | | | | | | | | GCAATTCAGTT |
| site | rs111033264 | c.10561T > C | p.(Trp3521Arg) | 13.20 | NNNRRT | SaCas9 | C8 | TCTTGAAGATACA |
| C9 | | | | | | KKH | | ATTGTCTTAAACCG |
| | | | | | | | | GAGAAAACCTATA |
| | | | | | | | | CAATCAAATGg |
| site | — | c.2296T > C | p.(Cys766Arg) | 1.71 | NGA | SpCas9VRQ | C7 | ATTCTGCAATCCTC |
| C10 | | | | | | R | | ACTCTGGGCAGCG |
| | | | | | | | | TGAGTGCAAAAAA |
| | | | | | | | | GAAGCCAAAGG |
| site | rs201657446 | c.7595- | p.(Pro2533Asnfs*5) | 29.95 | NGA | SpCas9VRQ | C3 | CGGAGGAACTACA |
| C11 | | 3C > G | | | | R | | GGTCCAGGTTCTCT |
| | | | | | | | | AAAGTAAAATAAA |
| | | | | | | | | TCCAGAAAGTC |

TABLE 5
Adenine base editing for reversion of particular mutations in USH2A in HEK293
cells. This table shows guide sequence-base editor combinations that show high editing
efficiency. Top to bottom, the sequences in this table correspond to SEQ ID NOs: 12-35. See
FIGS. 3A and 3B for additional information about the loci described in this table.

| | | | | ABE7.10 | ABESe | | | | Sequence |
|---|---|---|---|---|---|---|---|---|---|
| Exp | | | Amino Acid | Editing | Editing | | | editing | (SEQ ID NOs: 12- |
| # | dnSNP | DNA | Substitution | rate(%) | rate(%) | PAM | Cas9 var | window | 35) |
| site | rs111033364 | c.11864G > A | p.(Trp3955*) | 29.22 | 89.45 | NGG | SpCas9WT | A3 | TCCAAGGGTTCA |
| A1 | | | | | | | | | GTGGAGAGTCTG |
| | | | | | | | | | TAGTCATTAACA |
| | | | | | | | | | CAAACTCTGGAA |
| | | | | | | | | | GCT |
| site | rs202175091 | c.10712C > T | p.(Thr3571Met) | 16.94 | 65.13 | NGA | SpCas9 | A6 | GCTACTGGTGGC |
| A2 | | | | | | | VRQR | | ACAGCCAGCAA |
| | | | | | | | | | CCATGCAAGCTT |
| | | | | | | | | | TCAGCTGATATG |
| | | | | | | | | | AATA |
| site | rs111033334 | c.2209C > T | p.(Arg737*) | 1.26 | 1.85 | NNNRRT | SaCas9 | A6 | TCACATCCAACA |
| A3 | | | | | | | KKH | | TCATTAAAGCTT |
| | | | | | | | | | CAGAGAAATTTA |
| | | | | | | | | | AATCCAAAATTG |
| | | | | | | | | | CAA |
| site | rs199605265 | c.12575G > A | p.(Arg4192His) | 29.14 | 75.82 | NGA | SpCas9VR | A7 | AAAATAATTCGC |
| A4 | | | | | | | QR | | TATGAAGTGATT |
| | | | | | | | | | CACAGATGCTTC |
| | | | | | | | | | GAGGGAAAAGC |
| | | | | | | | | | TTGG |
| site | rs146733615 | c.14803C > T | p.(Arg4935*) | 69.83 | 82.85 | NGG | SpCas9WT | A5 | TTGCTGTCCACC |
| A5 | | | | | | | | | GAAAATGGGGC |
| | | | | | | | | | TCAGTACTGAGG |
| | | | | | | | | | CACTGTGGGGAG |
| | | | | | | | | | AAAG |
| site | rs199679165 | c.4645C > T | p.(Arg1549*) | 0.00 | 3.93 | NNNRRT | SaCas9 | A4 | ATCAAACCTTCA |
| A6 | | | | | | | KKH | | GGCACTTTTGTT |
| | | | | | | | | | CAAAAGCTGGCC |
| | | | | | | | | | TTAATGCCTGGT |
| | | | | | | | | | AAG |
| site | rs772808534 | c.100C > T | p.(Arg34*) | 36.66 | 81.60 | NGA | SpCas9 | A8 | TTCTCCAGCCTT |
| A7 | | | | | | | VRQR | | GGGAAAAGACC |
| | | | | | | | | | TCATGACTCAGT |
| | | | | | | | | | CAAGGATATTGA |
| | | | | | | | | | AGCA |
| site | rs201238640 | c.13274C > T | p.(Thr4425Met) | 37.61 | 82.56 | NGA | SpCas9 | A7 | CACACTAGCTGT |
| A8 | | | | | | | VRQR | | GCAACCTCCATT |
| | | | | | | | | | CATGCAGGCTAC |
| | | | | | | | | | AAGGGAGAAGT |
| | | | | | | | | | TATA |
| site | rs753330544 | c.13316C > T | p.(Thr4439Ile) | 25.64 | 38.10 | NGA | SpCas9 | A7 | CATGTTCTCTGG |
| A9 | | | | | | | VRQR | | CAGGGCCTCCAT |
| | | | | | | | | | TATCCAGGCAGA |
| | | | | | | | | | TTTTGACACACT |
| | | | | | | | | | AGC |
| site | rs111033280 | c.802G > A | p.(Gly268Arg) | 0.00 | 0.20 | NNNRRT | SaCas9 | A5 | atttgcagGTTTA |
| A10 | | | | | | | KKH | | GAGCAGTTTGTCA |
| | | | | | | | | | GAAGAATGCAAG |
| | | | | | | | | | ATTTTCGATTAT |
| | | | | | | | | | A |
| site | rs397517963 | c.1000C > T | p.(Arg334Trp) | 0.63 | 8.09 | NG | SpCas9-NG | A5 | AGAGGATGGGC |
| A11 | | | | | | | | | TTCAGGATTCAA |
| | | | | | | | | | CCATGACACTCT |
| | | | | | | | | | ATTATCAGCTGT |
| | | | | | | | | | GTCT |
| site | rs534534437 | c.1876C > T | p.(Arg626*) | 0.18 | 40.27 | NG | SpCas9-NG | A6 | GCCGAAGGATCT |
| A12 | | | | | | | | | GCACCAACTTGT |
| | | | | | | | | | CAGAAAAAGTA |
| | | | | | | | | | ATCCTTGCACAG |
| | | | | | | | | | CTCA |
| site | — | c.2440C > T | p.(Gln814*) | 1.51 | 6.35 | NGA | SpCas9VR | A3 | TCAACATTGGGC |
| A13 | | | | | | | QR | | TTGCAGATGCAC |
| | | | | | | | | | TACCCTGTCTTA |
| | | | | | | | | | GCATTACAGACA |
| | | | | | | | | | GTC |
| site | rs13947 | c.2797C > T | p.(Gln933*) | 0.00 | 10.39 | NGA | SpCas9VR | A4 | ATTTCTTTCTTAC |
| A14 | 37087 | | | | | | QR | | CTGGTTGACACT |
| | | | | | | | | | AATTACACCTTC |
| | | | | | | | | | TTCCTTGACGAT |
| | | | | | | | | | TA |
| site | rs764797292 | c.3883C > T | p.(Arg1295*) | 4.40 | 49.47 | NGG | SpCas9WT | A6 | AGCCAACCACTG |
| A15 | | | | | | | | | CTCTGAAAAACT |
| | | | | | | | | | CAACTTTCCTCA |
| | | | | | | | | | GATGTGGTTTCT |
| | | | | | | | | | TTA |
| site | rs754768875 | c.4957C > T | p.(Arg1653*) | 2.26 | 12.39 | NGA | SpCas9VR | A7 | TCCTTCCTGAGG |
| A16 | | | | | | | QR | | ATGGTATAACTT |
| | | | | | | | | | CACGGGAGCCCT |
| | | | | | | | | | CCCAGAAAGACT |
| | | | | | | | | | CCT |
| site | rs375668376 | c.5581G > A | p.(Gly1861Ser) | 10.56 | 54.03 | NG | SpCas9-NG | A7 | cccttactatttg |
| A17 | | | | | | | | | ctagGTTTCGGTA |
| | | | | | | | | | GTTGCATGAAGGA |
| | | | | | | | | | TGTTAAATTTAC |
| site | rs200712760 | c.8167C > T | p.(Arg2723*) | 6.81 | 32.69 | NGG | SpCas9WT | A9 | ACAGGTGGCTGC |
| A18 | | | | | | | | | ACCCCAGCAGGT |
| | | | | | | | | | CATGAGGGTCTT |
| | | | | | | | | | GTGGTAACTTCT |
| | | | | | | | | | ACC |
| site | — | c.9874C > T | p.(Gln3292*) | 5.39 | 60.50 | NG | SpCas9-NG | A4 | ACAATCTGTCTG |
| A19 | | | | | | | | | CCACAGCACTTC |
| | | | | | | | | | TAGCCATGGCCA |
| | | | | | | | | | TCATGAAGCCTC |
| | | | | | | | | | CCA |
| site | rs764182950 | c.9815C > T | p.(Pro3272Leu) | 63.68 | 60.76 | NGG | SpCas9WT | A5 | AATCTGGTTTCC |
| A20 | | | | | | | | | TGAGGTGGAGTA |
| | | | | | | | | | CAGCATTCTGCC |
| | | | | | | | | | ACAGCAGGAAT |
| | | | | | | | | | CACC |
| site | rs527236137 | c.13010C > T | p.(Thr4337Met) | 10.04 | 64.05 | NG | SpCas9-NG | A7 | TTTGCTGGTGGA |
| A21 | | | | | | | | | GCATCCTCCACT |
| | | | | | | | | | CATGCAGGCTTG |
| | | | | | | | | | GAGTGCATAGCT |
| | | | | | | | | | ATA |
| site | rs367674026 | c.13822C > T | p.(Arg4608*) | 1.26 | 24.36 | NGG | SpCas9WT | A6 | CCCAGGGTGGTG |
| A22 | | | | | | | | | CACGCTTGAATT |
| | | | | | | | | | CATATTTCATAC |
| | | | | | | | | | CTTCAGGACATA |
| | | | | | | | | | AGG |
| site | rs757676723 | c.15017C > T | p.(Thr5006Met) | 2.21 | 27.39 | NG | SpCas9-NG | A5 | AGAGGTATCATA |
| A23 | | | | | | | | | TTGGATCAACGG |
| | | | | | | | | | CATCTTAACACT |
| | | | | | | | | | TCCTTCGTCAGT |
| | | | | | | | | | CGT |
| site | — | c.2755C > T | p.(Gln919*) | 0.74 | 22.01 | NG | SpCas9-NG | A9 | TGACGATTAGGC |
| A24 | | | | | | | | | ACACACAGGCA |
| | | | | | | | | | CTAGCCACTGAT |
| | | | | | | | | | TGGGTCACAAAT |
| | | | | | | | | | GGTC |

TABLE 6
Cytosine and adenine base editing for introduction of exon
skipping at exon splice acceptor sites (SAS) and splice
donor sites (SDS) in USH2A. Top to bottom, the Guide 5′→3′
spacer sequences in this table correspond to SEQ ID NOs:
36-63 and 92. (“BE4” indicates “BE4max”.)

| | | SDS | | % Efficiency | Guide 5′---->3′ | |
|---|---|---|---|---|---|---|
| | | or | Base | in WERi-RB | Spacer (SEQ ID | |
| Exon | CDS loci | SAS | Editors | cells | NOs: 36-63, 198) | PAM |
| 13 | C.2168-2A | SAS | ABE8e | 21 | GCUUUAGGGCUUAGG | catt |
| | | | | | UGUGAU | |
| 16 | C.3158-2A, | SAS | BE4-VRQR, | 35 | GGAGcUaaaUUacaa | agag |
| | C.3158-1G | | BE3-VRQR | | Ugaag | |
| 24 | C.4886-1G | SAS | BE4-VRQR | 25 | GCAGAGGAACcUaga | ggagat |
| | | | | | gaaga | |
| 28 | C.5573-1G | SAS | BE4 | 12 | CCGAAACCUAGCAAA | agggat |
| | | | | | UAGUA | |
| 43 | C.8559-2A | SAS | BE4 | 28 | AUCUAAAGCAAAAGA | agg |
| | | | | | CAAGC | |
| 45 | C.8846-2A | SAS | BE4 | 12 | CAGCUGUCAACAAUA | agg |
| | | | | | AAUGC | |
| 50 | C.9740-2A | SAS | BE4, BE4- | 2 | UUCACCUGUCAAUUU | aataat |
| | | | SaKKH | | AGGAC | |
| 56 | C.10940-2A | SAS | BE4-VRQR | 19 | AGACCUAGAAAAAGC | agaa |
| | | | | | AAGCA | |
| 59 | C.11390-2A | SAS | BE4-VRQR | 30 | AUCCCUUUAAAGAGA | agag |
| | | | | | GAGAG | |
| 62 | C.12067-2A | SAS | BE4 | 21 | UGUUCCCUGUAAGAA | acaggt |
| | | | | | AAUUA | |
| 69 | C.14969-2A | SAS | BE4, ABE | 0 | GGCUAGACAAAAGGA | tgg |
| | | | 7.10 | | AGAAC | |
| 13 | C.2168-1G | SAS | ABE-NRTH | Optimize and | GUAUCUUUAGGGCUU | GATC |
| | | | | Test | AGGUGU | |
| 13 | C.2168-1G | SAS | BE4-NRRH | Optimize and | CCCUAAAGAUAAAAU | TAAA |
| | | | | Test | AUAUU | |
| 18 | C.4081+1G | SDS | BE4-SaKKH | to be tested | AAUUAUUCCUAGAGA | aatagt |
| | | | | | AAUUA | |
| 21 | C.4397-2A, | SAS | ABE 7.10 | to be tested | AUUUAGCACCAGCAC | agg |
| | C.4397-1G | | | | AACUG | |
| 29 | C.5777-2A | SAS | BE4-SaKKH | to be tested | UGGAGCUGUGAAGGA | agag |
| | | | | | AUAAA | |
| 30 | C.5858-2A | SAS | BE4-VRQR, | to be tested | UUUAUUAUAGGCAUA | agg |
| | | | BE4 | | UUGAC | |
| 31 | C.6050-2A | SAS | ABE 7.10 | to be tested | UCUUGUGGGGCUGUG | aaagat |
| | | | | | GAAGA | |
| 32 | C.6164-2A | SAS | BE4-SaKKH | to be tested | AUUACCUACUGAUUA | gaaaat |
| | | | | | AAAAA | |
| 38 | C.7121-2A | SAS | BE4-SaKKH | to be tested | AUCUAAAGCAAAAGA | agg |
| | | | | | CAAGC | |
| 69 | C.14969-1G | SAS | ABE 7.10 | to be tested | GGCUAGACAAAAGGA | tgg |
| | | | | | AGAAC | |
| 69 | C.15052+1G | SDS | ABE 7.10 | to be tested | CCUUUUUCUUCCGAU | AGG |
| | | | | | CUGUU | |
| 71 | C.15298-2A | SAS | BE4-SaKKH | to be tested | CCCCUGAGAAGGAAG | gtgagt |
| | | | | | UUGCU | |
| 28 | C.5573-1G | SAS | BE4 | to be tested | CGAAACCUAGCAAAU | ggg |
| | | | | | AGUAA | |
| 28 | C.5573-1G | SAS | BE4 | to be tested | CCGAAACCUAGCAAA | agg |
| | | | | | UAGUA | |
| 28 | C.5776+1G | SDS | BE4 | to be tested | GAAACCUAGCAAAUA | ggat |
| | | | | | GUAAG | |
| 50 | C.9740-1G | SAS | BE4, BE4- | to be tested | UACUUCACCUGUCAA | gacaat |
| | | | SaKKH | | UUUAG | |
| 50 | C.9958+1G | SDS | BE4, BE4- | to be tested | CAUACUUCACCUGUC | agg |
| | | | SaKKH | | AAUUU | |
| 25 | C.4988-2A | SAS | ABE 7.10 | to be tested | CCUGCAGAGAUAAUC | agg |
| | | | | | CAAAA | |

### Methods

### Sequencing and Data Analysis

Genomic DNA was amplified by PCR using universal primers found in the edited portion of the transgene, was purified by QlAquick PCR Purification Kit (Qiagen), and was submitted for Sanger sequencing and Amplicon sequencing (CCIB DNA Core Facility at Massachusetts General Hospital, Cambridge, Mass.). For Sanger sequencing, editing effciency was quantified by EditR (https://moriaritylab.shinyapps.io/editrv10/). The result of amplicon sequencing was analyzed by CRISPResso2 (http://crispresso.pinellolab.partners.org/). The CRISPRessoWGS Linux program was run with the following parameters: minimum homology for alignment to an amplicon 60%, quantificationwindowcenter −10, quantificationwindowsize 20. For productive edit calculations, custom scripts and Microsoft Excel were used to evaluate and tabulate if each read coded for the exact protein sequence desired, without additional missense changes, nonsense changes, or indels.

### Functional USH2A Protein Restoration After Base Editing

1.5×10⁵ HEK293 cells were seeded into 48-well cell culture plates, transfected with 160 ng of ABE8 plasmid vector, 60 ng of sgRNA plasmid vector and 100 ng of USH2A WT or mutant (A1, A7, A12, A15) plasmid vector. Two days after transfection, cells were fixed with 1% paraformaldehyde (PFA) for 30 seconds, were blocked in 1% bovine serum albumin and 10% goat serum in PBS (pH 7.4) and were stained overnight with USH2A (1:4000) antibody that recognizes the intracellular region of USH2A 7, followed by labeling with Alexa Fluor 594-conjugated secondary antibody and Hoechst 33342 for nuclear staining. Images were taken with a fluorescent microscope (Eclipse Ti, Nikon, Tokyo, Japan). Cells positive for full length USH2A were counted in 6 fields per sample by Image J software (National Institute of Mental Health, MD, USA). (Co-transfection with a GFP expression plasmid confirmed a high transfection rate. However, this GFP signal was no longer visible after permeabilization with the very mild fixation conditions described above which are required for use of the USH2A antibody.)

### Example 3

### Monkey Transfection Experiments

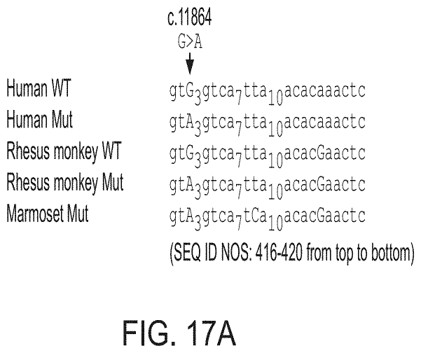

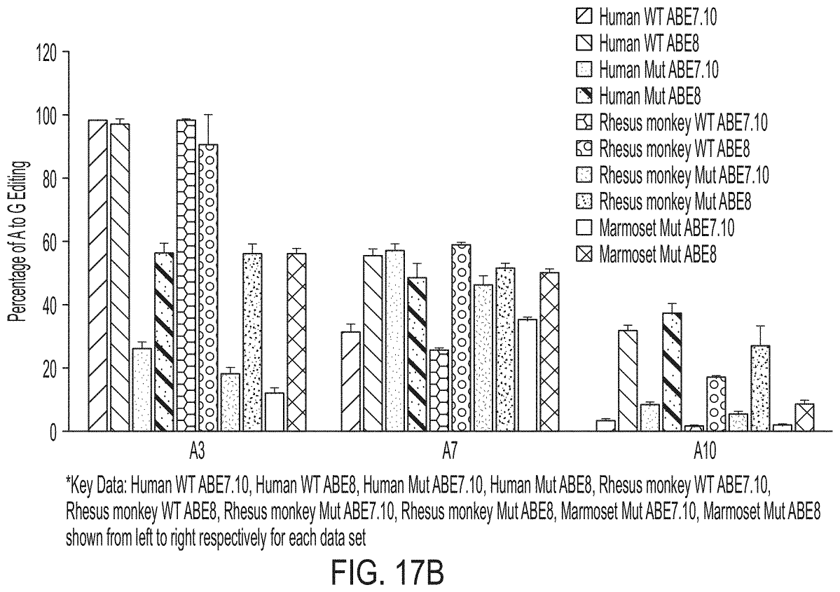

The potential of the wildtype rhesus monkey to serve as a testbed for estimating the efficiency of editing at the Al target adenine base was evaluated. Plasmids were created which contained the rhesus monkey, marmoset monkey, and human-specific USH2A editing targets shown in FIG. 17A. The plasmids contained wild-type and mutant human sequences, wild-type and mutant rhesus sequences, and a mutant marmoset sequence. Rhesus monkey ells were transiently transfected by vectors encoding the editing targets, a base editor (BE), and sgRNA. After transient transfection, each target sequence was amplified by PCR followed by Sanger sequencing to quantify the editing efficiency of the target base.

Editing efficiencies (% of A to G editing) at sites A3, A7, and A10 following administration of the ABE7.10, ABE8, are shown in FIG. 17B. Based on the editing efficiency of A7 in monkey study (in vivo), the editing efficiency of pathological mutations in human HEK293 cells may be translatable to monkey subjects because the editing efficiency of A7 in human mutated sequences is expected to be similar to that in monkey wild-type sequences. The site A1 51 bp sequence in human and monkey is homologous. These results are encouraging for the evaluation of these base editors in a non-human primate study.

TABLE 10
Primer sequence for cloning of sgRNA

| Target | Forward 5′->3′ | Reverse 5′->3′ | | |
|---|---|---|---|---|
| site | (SEQ ID NOS: | (SEQ ID NOS: | Type of | |
| name | 448-483) | 484-519) | Cas9 | PAM |
| C1 | CACCGCTCTTACCTTGGGAAA | AAACCTCCTTTCCCAAGGTAA | S. | NGG |
| | GGAG | GAGC | pyogenes | |
| C2 | CACCGTTCCTTCCCGCATCAG | AAACTTCCCTGATGCGGGAAG | S. | NGG |
| | GGAA | GAAC | pyogenes | |
| C3 | CACCGTTCCCCGTAAGAAAAT | AAACGTTAATTTTCTTACGGG | S. | NGG |
| | TAAC | GAAC | pyogenes | |
| C4 | CACCGATCCAAAGCAAAAGAC | AAACGCTTGTCTTTTGCTTTG | S. | NGG |
| | AAGC | GATC | pyogenes | |
| C5 | CACCGATTGGTGATTCCCGCT | AAACCCACAGCGGGAATCACC | S. aureus | NNG |
| | GTGG | AATC | | AAT |
| C6 | CACCGTAACAGGAATATTTGG | AAACTATACCAAATATTCCTG | S. | NGG |
| | TATA | TTAC | pyogenes | |
| C7 | CACCGTAGACGCCTCTGCTCC | AAACCCTGGGAGCAGAGGCGT | S. | NGA |
| | CAGG | CTAC | pyogenes | |
| C8 | CACCGATGAGTCTACTATTAC | AAACGAGAGTAATAGTAGACT | S. aureus | NNN |
| | TCTC | CATC | | AAT |
| C9 | CACCGCTTAAACCGGAGAAAA | AAACTAGGTTTTCTCCGGTTT | S. aureus | NNN |
| | CCTA | AAGC | | AAT |
| A1 | CACCGTAGTCATTAACACAAA | AAACGAGTTTGTGTTAATGAC | S. | NGG |
| | CTC | TAC | pyogenes | |
| A2 | CACCGCAACCATGCAAGCTTT | AAACGCTGAAAGCTTGCATGG | S. | NGA |
| | CAGC | TTGC | pyogenes | |
| A3 | CACCGCTTCAGAGAAATTTAA | AAACGATTTAAATTTCTCTGA | S. aureus | NNN |
| | ATC | AGC | | AAT |
| A4 | CACCGTGATTCACAGATGCTT | AAACCTCGAAGCATCTGTGAA | S. | NGA |
| | CGAG | TCAC | pyogenes | |
| A5 | CACCGCTCAGTACTGAGGCAC | AAACACAGTGCCTCAGTACTG | S. | NGG |
| | TGT | AGC | pyogenes | |
| A6 | CACCGTTCAAAAGCTGGCCTT | AAACCATTAAGGCCAGCTTTT | S. aureus | NNN |
| | AATG | GAAC | | GGT |
| A7 | CACCGAGACCTCATGACTCAG | AAACTTGACTGAGTCATGAGG | S. | NGA |
| | TCAA | TCTC | pyogenes | |
| A8 | CACCGCCATTCATGCAGGCTA | AAACCTTGTAGCCTGCATGAA | S. | NGA |
| | CAAG | TGGC | pyogenes | |
| A9 | CACCGTCCATTATCCAGGCAG | AAACAAATCTGCCTGGATAAT | S. | NGA |
| | ATTT | GGAC | pyogenes | |
| A10 | CACCGTGTCAGAAGAATGCAA | AAACAATCTTGCATTCTTCTG | S. aureus | NNN |
| | GATT | ACAC | | GAT |
| Dnmt1 | CACCGCGGGCTGGAGCTGTTC | AAACGCGCGAACAGCTCCAGC | S. | NGG |
| | GCGC | CCGC | pyogenes | |
| C10 | CACCGGGCAGCGTGAGTGCAA | AAACTTTTTGCACTCACGCTG | S. | NGA |
| | AAA | CCC | pyogenes | |
| C11 | CACCGCTCTAAAGTAAAATAA | AAACGGATTTATTTTACTTTA | S. | NGA |
| | ATCC | GAGC | pyogenes | |
| A1l | CACCGAACCATGACACTCTAT | AAACGATAATAGAGTGTCATG | S. | NGC |
| | TATC | GTTC | pyogenes | |
| A12 | CACCGTTGTCAGAAAAAGTAA | AAACAGGATTACTTTTTCTGA | S. | NGC |
| | TCCT | CAAC | pyogenes | |
| A13 | CACCGCTACCCTGTCTTAGCA | AAACGTAATGCTAAGACAGGG | S. | NGA |
| | TTAC | TAGC | pyogenes | |
| A14 | CACCGACTAATTACACCTTCT | AAACAGGAAGAAGGTGTAATT | S. | NGA |
| | TCCT | AGTC | pyogenes | |
| A15 | CACCGAACTCAACTTTCCTCA | AAACCATCTGAGGAAAGTTGA | S. | NGG |
| | GATG | GTTC | pyogenes | |
| A16 | CACCGAACTTCACGGGAGCCC | AAACGGGAGGGCTCCCGTGAA | S. | NGA |
| | TCCC | GTTC | pyogenes | |
| A17 | CACCGTTCGGTAGTTGCATGA | AAACTCCTTCATGCAACTACC | S. | NGT |
| | AGGA | GAAC | pyogenes | |
| A18 | CACCGAGCAGGTCATGAGGGT | AAACCAAGACCCTCATGACCT | S. | NGG |
| | CTTG | GCTC | pyogenes | |
| A19 | CACCGTCTAGCCATGGCCATC | AAACTCATGATGGCCATGGCT | S. | NGC |
| | ATGA | AGAC | pyogenes | |
| A20 | CACCGTACAGCATTCTGCCAC | AAACGCTGTGGCAGAATGCTG | S. | NGG |
| | AGC | TAC | pyogenes | |
| A21 | CACCGCCACTCATGCAGGCTT | AAACCTCCAAGCCTGCATGAG | S. | NGC |
| | GGAG | TGGC | pyogenes | |
| A22 | CACCGAATTCATATTTCATAC | AAACGAAGGTATGAAATATGA | S. | NGG |
| | CTTC | ATTC | pyogenes | |
| A23 | CACCGCGGCATCTTAACACTT | AAACAAGGAAGTGTTAAGATG | S. | NGT |
| | CCTT | CCGC | pyogenes | |
| A24 | CACCGCAGGCACTAGCCACTG | AAACCAATCAGTGGCTAGTGC | S. | NGT |
| | ATTG | CTGC | pyogenes | |

TABLE 11
Primer sequences.

| Primer | sequence (5′-->3′) |
|---|---|
| name | (SEQ ID NOS: 520-536) |
| M13-F | CCCAGTCACGACGTTGTAAAACG |
| M13-R | AGCGGATAACAATTTCACACAGG |
| AOX-F | GACTGGTTCCAATTGACAAGC |
| AOX-R | GCAAATGGCATTCTGACATCC |
| Luc-F | AGTCAAGTAACAACCGCGA |
| Luc-R | CCTTATGCAGTTGCTCTCC |
| Bglo-F | CTGGTCATCATCCTGCCTTT |
| Bglo-R | GCCCCCTCCATATAACA |
| CMV-F | AAATGGGCGGTAGGCGTG |
| XL39-R | ATTAGGACAAGGCTGGTGGG |
| tdTomat0-F | CTGTTCCTGTACGGCATGG |
| tdTomato-R | CTTTGATGACGGCCATGT |
| GW-F | AATCTCGCCGGATCCTAACT |
| GW-R | GCATGATGACCACCGATATG |
| PuroDNR-1F | GGTCCACTAGTGCTGTCCTG |
| AAVS-1R | AGGTTCCGTCTTCCTCCACT |
| AAVS-2F | CTTGCCAAGGACTCAAACCC |

TABLE 12
Primer set used to check off-target sequence
and size of the PCR product.

| | | length |
|---|---|---|
| | | of PCR |
| | 5′-->3′ | product |
| Primer name | (SEQ ID NOS: 537-552) | (bp) |
| A-m1_5p_fwd | TGTCAGATACCATATGCTGCTCAGCTTC | 1882 |
| | CAGTGGAGAGTCTAAAG | |
| A-m1_5p_rev | GTTAATGACTACAGACTCTCCACTGAAC | |
| A-m1_3p_fwd | GAGAGTCTGTAGTCATTAACACAAACTC | 687 |
| | TGG | |
| A-m1_3p_rev | GTTAACAGGCTCAGACCAGCTCAGCTCA | |
| | ACACTGGTGGACTTC | |
| A-m7_5p_fwd | CAGATCCGCTAGAGATCCGCGGCCGCCA | 139 |
| | CCATGAATTGCC | |
| A-m7_5p_rev | AAAGACCTCATGACTCAGTCAAGGATAT | |
| | TGAAGC | |
| A-m7_3p_fwd | GACTGAGTCATGAGGTCTTTTCCCAAGG | 857 |
| | CTGGAG | |
| A-m7_3p_rev | GTGCCAAAGGGTGGACCCGCGGGTGGCT | |
| | GCCAGGGCAACG | |
| A-m12_5p_fwd | CAGATCCGCTAGAGATCCGCGGCCGCCA | 1915 |
| | CCATGAATTGCCCAGTTC | |
| A-m12_5p_rev | CAACTTGTCAGAAAAAGTAATCCTTGCA | |
| | CAGC | |
| A-m12_3p_fwd | TTACTTTTTCTGACAAGTTGGTGCAGAT | 156 |
| | C | |
| A-m12_3p_rev | TTGCACTGCCTGCCAGACACGTGTCTCT | |
| | TACAATTACACTGTCCTC | |
| A-m15_5p_fwd | AGTGTAATTGTAAGAGACACGTGTCTGG | 1912 |
| | CAGGCAGTGCAATCAG | |
| A-m15_5p_rev | TGAAAAACTCAACTTTCCTCAGATGTGG | |
| | TTTCTTTAG | |
| A-m15_3p_fwd | GAGGAAAGTTGAGTTTTTCAGAGCAGTG | 1308 |
| A-m15_3p_rev | AAGTTCCAGGAACCCTGCTCCTAGGAAC | |
| | TGAGCTCCCTCATTTAATG | |

### REFERENCES

Each of the below references is incorporated herein in its entirety.
1. Fokkema, I. F. et al. LOVD v.2.0: the next generation in gene variant databases. Hum Mutat 32, 557-563, doi:10.1002/humu.21438 (2011).
2. Stenson, P. D. et al. The Human Gene Mutation Database (HGMD((R))): optimizing its use in a clinical diagnostic or research setting. Hum Genet 139, 1197-1207, doi:10.1007/s00439-020-02199-3 (2020).
3. Levy, J. M. et al. Cytosine and adenine base editing of the brain, liver, retina, heart and skeletal muscle of mice via adeno-associated viruses. Nat Biomed Eng 4, 97-110, doi:10.1038/s41551-019-0501-5 (2020).
4. Komor, A. C. et al. Improved base excision repair inhibition and bacteriophage Mu Gam protein yields C:G-to-T:A base editors with higher efficiency and product purity. Sci Adv 3, eaao4774, doi:10.1126/sciadv.aao4774 (2017).
5. Richter, M. F. et al. Phage-assisted evolution of an adenine base editor with improved Cas domain compatibility and activity. Nat Biotechnol 38, 883-891, doi:10.1038/s41587-020-0453-z (2020).
6. Tsai, S. Q. et al. CIRCLE-seq: a highly sensitive in vitro screen for genome-wide CRISPR-Cas9 nuclease off-targets. Nat Methods 14, 607-614, doi:10.1038/nmeth.4278 (2017).
7. Pendse, N. D. et al. In Vivo Assessment of Potential Therapeutic Approaches for USH2A-Associated Diseases. Adv Exp Med Biol 1185, 91-96, doi:10.1007/978-3-030-27378-115 (2019).

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.

What is claimed is:

1. A method for deaminating an adenosine (A) in an USH2A gene, the method comprising contacting the USH2A gene with a base editor in association with a guide RNA (gRNA), wherein the gRNA comprises a guide sequence that is complementary to a target nucleic acid sequence in the USH2A gene, wherein the base editor comprises a nucleic acid programmable DNA binding protein (napDNAbp) domain and an adenosine deaminase domain, wherein the guide sequence comprises a protospacer that comprises a nucleic acid sequence having at least 90%, at least 95%, at least 98%, or at least 99% identity to any one of SEQ ID NOs: 12-35, or a naturally-occurring variant thereof.

2. The method of claim 1, wherein the guide sequence comprises at least 10, 11, 12, 13, 14, 15, 16, 17, 18, 19, 20, 21, 22, 23, 24, or 25 contiguous nucleobases that are at least 90%, at least 95%, or 100% complementary to the target nucleic acid sequence of the USH2A gene.

3. The method of claim 1 or 2, wherein the guide sequence comprises a protospacer that comprises a nucleic acid sequence that differs by 1, 2, 3, 4, 5, 6, 7, 8, 9, or 10 nucleotides from the nucleotide sequence of any one of SEQ ID NOs: 12-35.

4. The method of any one of claims 1-3, wherein the guide sequence comprises a protospacer that comprises a nucleic acid sequence having at least 90%, at least 95%, at least 98%, or at least 99% identity to any one of SEQ ID NOs: 12, 15, 16, 18, and 19.

5. The method of any one of claims 1-4, wherein the guide sequence comprises a protospacer that comprises the nucleic acid sequence of any one of SEQ ID NOs: 12, 15, 16, 18, and 19.

6. The method of any one of claims 1-5, wherein the adenosine deaminase domain comprises a first adenosine deaminase selected from a wild-type TadA, an N-terminal truncated TadA, a TadA7.10, and a TadA-8e.

7. The method of any one of claims 1-6, wherein the first adenosine deaminase comprises an amino acid sequence having at least 90%, at least 95%, at least 98%, or at least 99% identity to any one of SEQ ID NOs: 79, 90, and 91.

8. The method of any one of claims 1-7, wherein the first adenosine deaminase comprises an amino acid sequence selected from the group consisting of SEQ ID NOs: 79, 90, and 91.

9. The method of any one of claims 1-8, wherein the first adenosine deaminase comprises the amino acid sequence of SEQ ID NO: 91.

10. The method of any one of claims 1-9, wherein the base editor is ABE7.10, ABE8e, ABE8e-SaKKH, ABE8e-NG, ABE-xCas9, ABE7.10-SaKKH, ABE7.10-NG, ABE7.10-VRQR, ABE8e-NRTH, ABE8e-NRRH, or ABE8e-VRQR.

11. The method of any one of claims 1-10, wherein the guide sequence comprises at least 15 contiguous nucleobases that are at least 90%, at least 95%, or 100% complementary to the target nucleic acid sequence of the USH2A gene.

12. The method of any one of claims 1-11, wherein the napDNAbp domain comprises a nuclease or a nickase.

13. The method of any one of claims 1-12, wherein the napDNAbp domain comprises a S. pyogenes Cas9 (SpCas9), a S. aureus Cas9 (SaCas9), or a variant thereof.

14. The method of any one of claims 1-13, wherein the napDNAbp domain comprises a wild-type SpCas9, a wild-type SaCas9, a SpCas9-VRQR, a SaCas9-KKH, a SpCas9-NG, an xCas9, a SpCas9-NRRH, or a SpCas9-NRTH.

15. The method of any one of claims 1-14, wherein the napDNAbp domain comprises a wild-type SpCas9 or a SpCas9-VRQR.

16. The method of any one of claims 1-15, wherein the USH2A gene comprises a C100T, a G802A, a C1000T, a C1876T, a C2209T, a C2440T, a C2755T, a C2797T, a C3883T, a C4645T, a C4957T, a G5581A, a C8167T, a C9874T, a C9815T, a C10712T, a G11864A, a G12575A, a C13010T, a C13274T, a C13316T, a C13822T, a C14803T, or a C15017T mutation.

17. The method of any one of claims 1-16, wherein the USH2A gene comprises a C100T, a G11864A, a G12575A, a C13274T, or a C14803T mutation.

18. The method of any one of claims 1-17, wherein the guide RNA comprises a nucleic acid sequence that is at least 90%, at least 95%, at least 98%, or at least 99% identical to any one of SEQ ID NOs: 36, 46, 47, 50, 53, 56, 57, and 93.

19. The method of any one of claims 1-18, wherein the guide RNA comprise a nucleic acid sequence that differs by 1, 2, 3, 4, 5, or 6 nucleotides from the nucleotide sequence of any one of SEQ ID NOs: 36, 46, 47, 50, 53, 56, 57, and 93.

20. The method of any one of claims 1-19, wherein the guide sequence comprises a nucleic acid selected from SEQ ID NOs: 36, 46, 47, 50, 53, 56, 57, and 93.

21. The method of any one of claims 1-20, wherein the guide sequence comprises a nucleic acid sequence selected from the group consisting of SEQ ID NOs: 36, 47, and 93.

22. The method of any one of claims 18-21, wherein the step of contacting results in the disruption of a splice site in the USH2A gene.

23. The method of any one of claims 18-22, wherein the step of contacting results in the disruption of a splice site in the USH2A gene at a splice acceptor site or a splice donor site associated with exon 13, exon 21, exon 25, exon 31, and exon 69.

24. The method of any one of claims 18-23, wherein the step of contacting results in the disruption of a splice acceptor site or a splice donor site associated with exon 13.

25. The method of any one of claims 18-24, wherein the step of contacting results in the skipping of exon 13 during splicing of an RNA transcript of the USH2A gene.

26. The method of any one of claims 1-25, wherein the guide RNA comprises a nucleic acid sequence that is at least 85%, at least 90%, at least 95%, at least 98%, or at least 99% identical to SEQ ID NO: 170.

27. The method of any one of claims 1-26, wherein the guide RNA comprises a first nucleic acid sequence that differs by 1, 2, 3, 4, 5, or 6 nucleotides from the nucleotide sequences of any one of any one of SEQ ID NOs: 36, 46, 47, 50, 53, 56, 57, 93, 209-213, 215-232, and 329, and a second nucleic acid sequence that differs by 1, 2, 3, 4, 5, 6, 7, 8, 9, or 10 nucleotides from the nucleotide sequence of SEQ ID NO: 170.

28. The method of any one of claims 1-27, wherein the guide RNA comprises a first nucleic acid sequence comprising any one of SEQ ID NOs: 36, 46, 47, 50, 53, 56, 57, 93, 209-213, 215-232, and 329, and a second nucleic acid sequence comprising SEQ ID NO: 170.

29. The method of any one of claims 1-28, wherein the method is performed in vivo.

30. The method of any one of claims 1-28, wherein the method is performed ex vivo.

31. The method of any one of claims 1-28, wherein the method is performed in vitro.

32. The method of any one of claims 1-29, wherein the method is performed in a subject.

33. The method of claim 32, wherein the method is performed in the inner ear of a subject.

34. The method of claim 32 or 33, wherein the subject has or is suspected of having Usher syndrome.

35. The method of any one of claims 32-34, wherein the subject is human.

36. The method of any one of claims 1-35, wherein the guide RNA comprises a first nucleic acid sequence comprising SEQ ID NO: 209 and a second nucleic acid sequence comprising SEQ ID NO: 170, wherein the base editor is ABE8e, and wherein the USH2A gene comprises a G11864A mutation.

37. The method of any one of claims 1-35, wherein the guide RNA comprises a first nucleic acid sequence comprising SEQ ID NO: 36 and a second nucleic acid sequence comprising SEQ ID NO: 170, wherein the base editor is an ABE8e, and wherein the step of contacting results in the disruption of a splice acceptor site or a splice donor site associated with exon 13.

38. A complex comprising (i) an adenosine base editor, and (ii) a guide RNA (gRNA) that comprises a nucleic acid sequence that is at least 90%, at least 95%, at least 98%, or at least 99% identical to any one of SEQ ID NOs: 12-36, 46, 47, 50, 53, 56, 57, 93, 209-213, 215-232, and 329.

39. The complex of claim 38, wherein the guide RNA comprises a nucleic acid sequence selected from the group consisting of SEQ ID NOs: 12-36, 46, 47, 50, 53, 56, 57, 93, 209-213, 215-232, and 329.

40. The complex of claim 38 or 39, wherein the guide RNA comprises a protospacer that comprises a nucleic acid sequence that is at least 90%, at least 95%, at least 98%, or at least 99% identical to any one of SEQ ID NOs: 210, 213, 214, 216, 217, 36, and 47.

41. The complex of any one of claims 38-40, wherein the base editor is ABE7.10, ABE8e, ABE8e-SaKKH, ABE8e-NG, ABE-xCas9, ABE7.10-SaKKH, ABE7.10-NG, ABE7.10-VRQR, ABE8e-NRTH, ABE8e-NRRH, or ABE8e-VRQR.

42. The complex of any one of claims 38-41, wherein the base editor comprises ABE8e.

43. The complex of claim 38, wherein the adenine base editor comprises ABE8e and the guide RNA comprises a first nucleic acid sequence of SEQ ID NO: 217 and a second nucleic acid sequence comprising SEQ ID NO: 170.

44. The complex of claim 38, wherein the adenine base editor comprises ABE8e-VRQR and the guide RNA comprises a first nucleic acid sequence of SEQ ID NO: 209 and a second nucleic acid sequence comprising SEQ ID NO: 170.

45. A method for deaminating a cytosine (C) in an USH2A gene, the method comprising contacting the USH2A gene with a cytidine base editor in association with a guide RNA (gRNA), wherein the gRNA comprises a guide sequence that is complementary to a target nucleic acid sequence in the USH2A gene, wherein the target nucleic acid sequence in the USH2A gene comprises a protospacer that comprises a nucleic acid sequence having at least 90%, at least 95%, at least 98%, or at least 99% identity to any one of of any one of SEQ ID NOs: 1-11, or a naturally-occurring variant thereof, wherein the base editor comprises a nucleic acid programmable DNA binding protein (napDNAbp) domain and a cytosine deaminase domain.

46. The method of claim 45, wherein the guide sequence comprises a protospacer that comprises a nucleic acid sequence that differs by 1, 2, 3, 4, 5, 6, 7, 8, 9, or 10 nucleotides from the nucleotide sequence of any one of SEQ ID NOs: 1-11.

47. The method of claim 45 or 46, wherein the guide sequence comprises a protospacer that comprises a nucleic acid sequence that comprises any one of SEQ ID NOs: 1-11.

48. The method of any one of claims 45-47, wherein the protospacer comprises a nucleic acid sequence comprises any one of SEQ ID NOs: 1, 2, or 7.

49. The method of any one of claims 45-47, wherein the cytosine deaminase domain comprises a member of the APOBEC family of deaminases.

50. The method of any one of claims 45-49, wherein the cytosine deaminase domain comprises an amino acid sequence having at least 90%, at least 95%, at least 98%, or at least 99% identity to any one of SEQ ID NOs: 292-326.

51. The method of any one of claims 45-50, wherein the cytosine deaminase domain comprises an amino acid sequence selected from the group consisting of SEQ ID NOs: 292-326.

52. The method of any one of claims 45-51, wherein the cytosine deaminase domain comprises the amino acid sequence of SEQ ID NO: 318.

53. The method of any one of claims 45-52, wherein the base editor is BE3, BE3.9max, BE4max, BE4-SaKKH, BE3.9-NG, BE3.9-NRRH, or BE4max-VRQR.

54. The method of any one of claims 45-53, wherein the base editor is BE4max.

55. The method of any one of claims 45-54, wherein the napDNAbp domain is a nuclease or a nickase.

56. The method of claim any one of claims 45-55, wherein the napDNAbp domain comprises a S. pyogenes Cas9 (SpCas9) or a S. aureus Cas9 (SaCas9), or a variant thereof.

57. The method of any one of claims 45-56, wherein the napDNAbp domain comprises a wild-type SpCas9, a wild-type SaCas9, a SpCas9-VRQR, a SaCas9-KKH, a SpCas9-NG, an xCas9, or a SpCas9-NRRH.

58. The method of any one of claims 45-57, wherein the napDNAbp comprises a wild-type SpCas9 or a SpCas9-VRQR.

59. The method of any one of claims 45-58, wherein the USH2A gene comprises a T1606C, an A1841G, a T2296C, an A3368G, a T4325C, an A7595G, an A8559G, a T9799C, a T10561C, or an A12067G mutation.

60. The method of any one of claims 45-59, wherein the guide RNA comprises a nucleic acid sequence that is at least 90%, at least 95%, at least 98%, or at least 99% identical to SEQ ID NOs: 37-45, 48, 49, 51, 52, 54, 55, 58-63, 93, and 198.

61. The method of any one of claims 45-60, wherein the guide RNA comprises a nucleic acid sequence selected from the group consisting of SEQ ID NOs: 37-45, 48, 49, 51, 52, 54, 55, 58-63, 93, and 198.

62. The method of any one of claims 45-61, wherein the guide RNA comprises a nucleic acid sequence that is at least 90%, at least 95%, at least 98%, or at least 99% identical to any one of SEQ ID NOs: 37, 40, and 44.

63. The method of any one of claims 45-62, wherein the guide RNA comprises a nucleic acid sequence selected from the group consisting of SEQ ID NOs: 37, 40, and 44.

64. The method of any one of claims 60-63, wherein the step of contacting results in the disruption of a splice site in the USH2A gene.

65. The method of any one of claims 60-64, wherein the step of contacting results in the disruption of a splice acceptor site or a splice donor site in the USH2A gene associated with exon 8, exon 13, exon 16, exon 17, exon 18, exon 21, exon 24, exon 28, exon 29, exon 30, exon 31, exon 32, exon 38, exon 43, exon 45, exon 50, exon 53, exon 56, exon 59, exon 62, exon 65, exon 68, exon 69, and exon 71.

66. The method of any one of claims 60-65, wherein the step of contacting results in the removal of a splice site at a splice acceptor site or a splice donor site associated with exon 16, exon 59, or exon 43.

67. The method of any one of claims 60-66, wherein the step of contacting results in the skipping of exon 16, exon 59, or exon 43 during splicing of an RNA transcript of the USH2A gene.

68. The method of any one of claims 45-67, wherein the guide RNA comprises a nucleic acid sequence that is at least 85%, at least 90%, at least 95%, at least 98%, or at least 99% identical to SEQ ID NO: 170.

69. The method of any one of claims 45-68, wherein the guide RNA comprises a first nucleic acid sequence that differs by 1, 2, 3, 4, 5, or 6 nucleotides from the nucleotide sequences of any one of any one of SEQ ID NOs: 199-209 and a second nucleic acid sequence that differs by 1, 2, 3, 4, 5, 6, 7, 8, 9, or 10 nucleotides from the nucleotide sequence of SEQ ID NO: 170.

70. The method of any one of claims 45-69, wherein the guide RNA comprises a first sequence comprising any one of SEQ ID NOs: 199-209 and a second sequence comprising SEQ ID NO: 170.

71. The method of any one of claims 45-70, wherein the method is performed in vivo.

72. The method of any one of claims 45-70, wherein the method is performed ex vivo.

73. The method of any one of claims 45-70, wherein the method is performed in vitro.

74. The method of any one of claims 45-70, wherein the method is performed in a subject.

75. The method of any one of claims 45-74, wherein the guide RNA comprises a first nucleic acid sequence comprising SEQ ID NO: 199 and a second nucleic acid sequence comprising SEQ ID NO: 170, wherein the base editor is BE4max, and wherein the USH2A gene comprises an A7595G mutation.

76. The method of any one of claims 1-37 and 45-75, wherein the step of contacting results in a sequence that is not associated with Usher syndrome.

77. The method of any one of claims 1-37 and 45-76, wherein the step of contacting leads to an increase in full-length Usherin protein.

78. The method of any one of claims 1-37 and 45-77, wherein the step of contacting partially or completely restores Usherin protein stability or function.

79. The method of any one of claims 1-37 and 45-78, wherein deaminating an adenosine or cytosine nucleobase in the USH2A gene partially or completely reverses visual or hearing loss.

80. A complex comprising (i) an cytidine base editor, and (ii) a guide RNA (gRNA) comprising a nucleic acid sequence that is at least 90%, at least 95%, at least 98%, or at least 99% identical to any one of SEQ ID NOs: 1-11, 37-45, 48, 49, 51, 52, 54, 55, 58-63, 93, and 198-209.

81. The complex of claim 80, wherein the guide RNA comprises a nucleic acid sequence selected from the group consisting of SEQ ID NOs: 1-11, 37-45, 48, 49, 51, 52, 54, 55, 58-63, 93, and 198-209.

82. The complex of claim 80 or 81, wherein the guide RNA comprises a nucleic acid sequence that is at least 90%, at least 95%, at least 98%, or at least 99% identical to any one of SEQ ID NOs: 199, 200, 205, 37, 40, and 44.

83. The complex of any one of claims 80-82, wherein the gRNA comprises a nucleic acid sequence selected from the group consisting of SEQ ID NOs: 199, 200, 205, 37, 40, and 44.

84. The complex of any one of claims 80-83, wherein the cytidine base editor is BE4max or BE4max-VRQR.

85. The complex of any one of claims 80-84, wherein the cytidine base editor comprises BE4max, and the gRNA comprises the nucleic acid sequence of SEQ ID NO: 199.

86. The complex of any one of claims 80-84, wherein the cytidine base editor comprises BE4max, and the gRNA comprises the nucleic acid sequence of SEQ ID NO: 40.

87. The complex of any one of claims 80-85, wherein the cytidine base editor comprises BE4max and the guide RNA comprises a first nucleic acid sequence of SEQ ID NO: 199 and a second nucleic acid sequence comprising SEQ ID NO: 170.

88. A pharmaceutical composition comprising the complex of any one of claims 38-44 and 80-87 further comprising a pharmaceutically acceptable excipient.

89. The pharmaceutical composition of claim 87 further comprising a cationic lipid or cationic polymer.

90. A vector comprising one or more polynucleotides encoding the complex of any one of claims 38-44 and 80-87.

91. The vector of claim 90, wherein the vector comprises a heterologous promoter driving expression of the gRNA.

92. The vector of claim 90 or 91, wherein the vector is a recombinant adeno-associated viral (rAAV) vector.

93. The vector of claim 92, wherein the rAAV vector is formulated for delivery to the retina or the inner ear of the subject by injection.

94. The vector of claim 93, wherein the rAAV vector is formulated for delivery by subretinal injection.

95. A cell comprising the complex of any one of claims 38-44 and 80-87, or the vector of any one of claims 90-94.

96. A method of treatment of a subject in need thereof comprising administering the complex of any one of claims 38-44 and 80-87, the vector of any one of claims 90-94, or the cell of claim 95 to the subject.

97. The method of claim 96, wherein the step of adminstering comprises a subretinal injection or injection into the inner ear.

98. The method of claim 96 or 97, wherein the subject is suffering from Usher syndrome or retinitis pigmentosa.

99. A kit comprising
(i) the vector of any one of claims 90-94; and
(ii) an expression construct encoding a guide RNA backbone, wherein the construct comprises a cloning site positioned to allow the cloning of a nucleic acid sequence identical or complementary to a target sequence into the guide RNA backbone.

100. Use of a complex of any one of claims 38-44 and 80-87, the pharmaceutical composition of any one of claims 87-89, or the cell of claim 95, as a medicament.

101. The use of claim 100, wherein the medicament is for treatment of Usher syndrome.

102. The use of claim 100, wherein the medicament is for treatment of autosomal recessive retinitis pigmentosa or of hearing loss.

103. A composition comprising:
(i) a first recombinant adeno associated virus (rAAV) particle comprising a first nucleotide sequence encoding a N-terminal portion of a base editor in accordance with the complex of any one of claims 38-44 and 80-87 fused at its C-terminus to an intein-N; and
(ii) a second recombinant adeno associated virus (rAAV) particle comprising a second nucleotide sequence encoding an intein-C fused to the N-terminus of a C-terminal portion of the base editor,
wherein at least one of the first nucleotide sequence and second nucleotide sequence is operably linked to a first promoter,
wherein at least one of the first nucleotide sequence and second nucleotide sequence comprises at its 3′ end a gRNA nucleic acid segment encoding a guide RNA in accordance with the complex of any one of claims 38-44 and 80-87 operably linked to a second promoter, and
wherein the direction of transcription of the guide RNA nucleic acid segment is reversed relative to the direction of transcription of the at least one nucleotide sequence.

104. The composition of claim 103, wherein the first rAAV particle and/or the second rAAV particle is an rAAV2 particle, rAAV6 particle, rAAV8 particle, rPHP.B particle, rPHP.eB particle, or rAAV9 particle, or a variant thereof.

105. The composition of claim 103 or 104, wherein the first rAAV particle and/or the second rAAV particle is an rAAV9 particle.

106. The composition of any one of claims 103-105, wherein the N-terminal portion of the base editor comprises a portion of any one of SEQ ID NOs: 350-388 that corresponds to amino acids 1-573 of the napDNAbp domain of the base editor.

107. The composition of any one of claims 103-106, wherein the C-terminal portion of the base editor comprises a portion of any one of SEQ ID NOs: 350-388 that corresponds to amino acids 574-1368 of the napDNAbp domain of the base editor.

108. A composition comprising a guide RNA that comprises a first nucleic acid sequence that differs by 1, 2, 3, 4, 5, or 6 nucleotides from the nucleotide sequences of any one of any one of SEQ ID NOs: 36-63, 198-232, and 329, and a second nucleic acid sequence that differs by 1, 2, 3, 4, 5, 6, 7, 8, 9, or 10 nucleotides from the nucleotide sequence of SEQ ID NO: 170.

109. The composition of claim 108, wherein the guide RNA comprises a first nucleic acid sequence comprising any one of SEQ ID NOs: 36-63, 198-232, and 329, and a second nucleic acid sequence comprising SEQ ID NO: 170.

110. The composition of claim 108 or 109, wherein the guide RNA comprises a first nucleic acid sequence comprising SEQ ID NO: 209 and a second nucleic acid sequence comprising SEQ ID NO: 170.

111. The composition of claim 108 or 109, wherein the guide RNA comprises a first nucleic acid sequence comprising SEQ ID NO: 36 and a second nucleic acid sequence comprising SEQ ID NO: 170.

112. The composition of claim 108 or 109, wherein the guide RNA comprises a first sequence comprising SEQ ID NO: 199 and a second sequence comprising SEQ ID NO: 170.

113. The composition of claim 108 or 109, wherein the guide RNA comprises a first nucleic acid sequence of SEQ ID NO: 217 and a second nucleic acid sequence comprising SEQ ID NO: 170.

114. The method of any one of claims 1-37, wherein the adenine base editor comprises an amino acid sequence having at least 95%, 98%, 99%, or 100% sequence identity to any one of SEQ ID NOs: 373-388.

115. The complex of any one of claims 38-44, wherein the adenine base editor comprises an amino acid sequence having at least 95%, 98%, 99%, or 100% sequence identity to any one of SEQ ID NOs: 373-388.

116. The method of any one of claims 45-79, wherein the cytidine base editor comprises an amino acid sequence having at least 95%, 98%, 99%, or 100% sequence identity to any one of SEQ ID NOs: 350-372.

117. The complex of any one of claims 80-87, wherein the cytidine base editor comprises an amino acid sequence having at least 95%, 98%, 99%, or 100% sequence identity to any one of 350-372.

118. The composition of any one of claims 103-107, wherein the base editor comprises an amino acid sequence having at least 95%, 98%, 99%, or 100% sequence identity to any one of SEQ ID NOs: 350-388.

119. The method of any one of claims 1-37, 45-79, 114, and 116, wherein the step of contacting partially or completely restores auditory function.

120. The method of any one of claims 1-37, 45-79, 114, and 116, wherein the step of contacting partially or completely restores auditory or visual function.
